(19) United States
(12) Patent Application Publication
(10) Pub. No.: US 2019/0054097 A1
(43) Pub. Date: Feb. 21, 2019

(54) COMPOSITIONS TARGETING SENESCENT CELLS AND THE USES THEREOF

(71) Applicant: BioVentures, LLC, Little Rock, AR (US)

(72) Inventors: Daohong Zhou, Gainesville, FL (US); Guangrong Zheng, Gainesville, FL (US); Xuan Zhang, Gainesville, FL (US); Yingying Wang, Chengdo (CN); Jianhui Chang, Chengdo (CN); Fen Xia, Little Rock, AR (US); Maria Schuller De Almeida, Little Rock, AR (US); Ha-Neui Kim, Little Rock, AR (US); Peiyi Zhang, Little Rock, AR (US)

(21) Appl. No.: 16/165,797

(22) Filed: Oct. 19, 2018

Related U.S. Application Data
(63) Continuation-in-part of application No. PCT/US2017/028875, filed on Apr. 21, 2017.
(60) Provisional application No. 62/325,856, filed on Apr. 21, 2016.
(60) Provisional application No. 62/575,068, filed on Oct. 20, 2017.
(60) Provisional application No. 62/575,015, filed on Oct. 20, 2017.

(51) Int. Cl. A61K 31/63; A61P 19/10; A61P 35/00

(52) U.S. Cl. CPC A61K 31/63; A61P 35/00; A61P 19/10

(57) ABSTRACT

The present disclosure provides compositions and methods for selectively killing senescent cells, wherein the composition comprises a compound of Formula (I) or a compound of Formula (II). The selective killing of senescent cells may delay aging and/or treat age-related disorders.

### CROSS REFERENCE TO RELATED APPLICATIONS

This application is a continuation-in-part of International Application No. PCT/US17/28875, filed Apr. 21, 2017 which claims the benefit of U.S. Provisional Application No. 62/325,856, filed Apr. 21, 2016, U.S. Provisional Application No. 62/575,068, filed Oct. 20, 2017, and U.S. Provisional No. 62/575,015, filed Oct. 20, 2017, the disclosures of which are hereby incorporated by reference in their entirety.

### FIELD OF THE INVENTION

The present disclosure relates to compositions and methods which target senescent cells. In part, the present disclosure provides compositions which inhibit or induce the degradation of anti-apoptotic Bcl-2 family proteins and their method of use in the treatment of various cancers and treatment and prevention of diseases and pathologies related to accumulation of senescent cells during aging, such as aging, cancer, chronic obstructive pulmonary disease (COPD), osteoporosis, osteoarthritis, atherosclerosis, neurodegenerative diseases, diabetes, and many others. The present invention also relates to pharmaceutical compositions containing these compounds as well as various uses thereof.

### BACKGROUND

Aging is the major risk factor for most functional deficits and many diseases in human, such as cancers, osteoarthritis, osteoporosis, atherosclerosis, neurodegenerative diseases, and diabetes. An increasing body of evidence demonstrates that aging is associated with an accumulation of senescent cells (Campisi, Cell 120:513-522, 2005; Campisi, Curr. Opin. Genet. Dev. 21:107-112, 2011; Rodier and Campisi, J. Cell Biol. 192:547-556, 2011). Senescent cell accumulation in tissues and organs is believed to cause tissue degradation and loss of function due to the increased levels of free radicals and various inflammatory mediators produced by senescent cells. Therefore, selective depletion of senescent cells may be a novel anti-aging strategy that may prevent cancer and various human diseases associated with aging and rejuvenate the body to live a healthier lifespan. This hypothesis is supported by recent findings that selective elimination of p16^{Ink4a} (p16)-positive senescent cells in BubR1 hypomorphic progeroid mouse model via a genetic approach extended the animals' healthy lifespan by delaying the onset of several age-related pathologies, such as cataracts, sarcopenia, and lordokyphosis (Baker et al., Nature 479:232-236, 2011 Baker et al., Nature 530:184-189, 2016). These studies validated the great therapeutic potential of targeting senescent cells.

The number of senescent cells increases in bone with age. Age-related bone loss is associated with an increase in the number of cells that resorb bone (osteoclasts) and a decrease in the cells that form bone (osteoblasts). Osteoblast senescence decreases cell number and increases the secretion of factors which promote osteoclast formation causing bones to become weak and brittle. Bone fractures caused by osteoporosis lead to severe restriction on activity. In particular, hip fracture is involved in high mortality of about 15 to 35%. Therefore, it is important to diagnose and treat osteoporosis prior to occurrence of osteoporotic fractures

Conventionally, bisphosphonate-based medicines have been known as medicines for treating osteoporosis. It is known that bisphosphonate sticks to an inorganic element of bone and when an osteoclast resorbs the bone to which bisphosphonate sticks, a non-hydrolyzed ATP analogue is formed and exhibits toxicity on the cell or causes a decrease in activity of the osteoclast and apoptosis in various ways in the osteoclast, thereby reducing bone resorption and thus increasing a bone density. Although such medicines have been known as being relatively safe, there have been recently suggested that when being used for a long time, the medicines may affect remodeling of bone by normal bone resorption or bone formation, or healing of bone after fracture, resulting in a decrease in bone elasticity and a bad effect on bone strength. There is a report that the medicines actually cause stress fractures in numerous patients.

Moreover, therapy induced-senescent cells may be important in a variety of conditions. Peripheral neuropathy (PN), characterized by pain and sensory loss in limbs, is a common side effect of chemotherapy. About 25 to 30% of cancer patients experience severe and persistent PN after chemotherapy, which can lead to reduced dose of the treatment agent, postponement of subsequent cycles of therapy, or even ceasing the treatment. This can reduce the therapeutic efficacy of chemotherapy. More importantly, persistent chemotherapy-induced PN (CIPN) reduces quality of life and imposes a heavy social and economic burden.

The peripheral nervous system (PNS) consists of sensory neurons running from stimulus receptors that inform the central nervous system (CNS) of the stimuli, and motor neurons running from the spinal cord to the effectors that take action. In CIPN, an anticancer drug could impair both sensory and motor functions. It can include sharp, stabbing pain, hearing loss, blurred vision and change in taste. In addition, the motor neuron dysfunction manifest as cramps, difficulty with fine motor activities (e.g. writing), gait disturbances, paralysis, spasms, tremors and weakness.

CIPN may result from the use of numerous chemotherapeutic agents, including, but not limited to, Ixabepilone (Ixempra Kit), arsenic trioxide (Trisenox), cytarabine (Cytosar-U, Depocyt, generics), etoposide, hexamethylmelamine (altretamine [Hexalen]), Ifosfamide (Ifex, generics), methotrexate (Trexall, generics), procarbazine (Matulane) and vinblastine. The chemotherapeutic drugs that most commonly elicit CIPN include platinum compounds (cisplatin, carboplatin, oxaliplatin), vincristine, taxanes (docetaxel, paclitaxel), epothilones (ixabepilone), bortezomib (Velcade), thalidomide (Thalomid) and lenalidomide.

CIPN symptoms are commonly managed in a manner similar to other types of nerve pain—that is, with a combination of physical therapy, complementary therapies such as massage and acupuncture, and medications that can include steroids, antidepressants, anti-epileptic drugs, and opioids for severe pain. Unfortunately, these therapies have not demonstrated true efficacy for treating CIPN, and virtually all of the drugs currently used to treat peripheral neuropathy carry side effects of their own. Moreover, the actual causes of CIPN, on the cellular and tissue level, remain largely a matter of speculation.

The Bcl-2 (B-cell lymphoma-2) family of proteins is a group of regulator proteins that plays a central role in regulating cell death by either inducing (pro-apoptotic) or inhibiting (anti-apoptotic) apoptosis. Anti-apoptotic Bcl-2 family of proteins, such as Bcl-2, Bcl-xL, Bcl-W, and Mcl-1, has been proven to be an attractive target for the development of novel anti-cancer agents (Lessene et al., Nat. Rev. Drug Discov. 7:989-1000, 2008; Vogler et al., Cell Death Differ. 2009; 16:360-367; Delbridge et al., Nat. Rev. Cancer 16:99-109, 2016). Numerous Bcl-2 small molecule inhibitors have been reported (Bajwa et al., Expert Opin. Ther. Patents 22:37-55, 2012; Vogler, Adv. Med. 1-14, 2014). The following are some of the Bcl-2 small molecule inhibitors that have been investigated at various stages of drug development: ABT-737 (US20070072860), navitoclax (ABT-263, WO2009155386), venetoclax (ABT-199, WO2010138588), obatoclax (GX 15-070, WO2004106328), (−)-gossypol (AT-101, WO2002097053), sabutoclax (BI-97C1, WO2010120943), TW-37 (WO2006023778), BM-1252 (APG-1252), and A-1155463 (WO2010080503). Venetoclax, a selective Bcl-2 inhibitor, was approved by the FDA in April 2016 for the treatment of chronic lymphocytic leukemia with 17-p deletion.

The Bcl-2 family of proteins has also been found to be a potential target for the development of “senolytic” drugs, drugs that targeting senescent cells for the delay of aging or treatment of aging-associated disease. For example, navitoclax (ABT-263), an inhibitor of Bcl-2, Bcl-xL, and Bcl-W, has been shown to selectively kill senescent cells in culture and deplete senescent cells in aged mice (WO2015171591; Chang et al., Nat. Med. 22:78-83, 2016; Zhu et al., Aging Cell 2016).

Accordingly, a need exists for the development of novel therapeutic options and methods of using the same which target senescent cells in a variety of conditions.

### SUMMARY

One aspect of the present disclosure encompasses compositions and methods of killing one or more senescent cells in a subject comprising administering a therapeutically effective amount of a composition as disclosed herein to a subject in need thereof.

Another aspect of the present disclosure provides a compound comprising Formula (II):

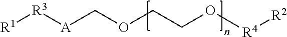

wherein

R¹ is selected from the group consisting of:

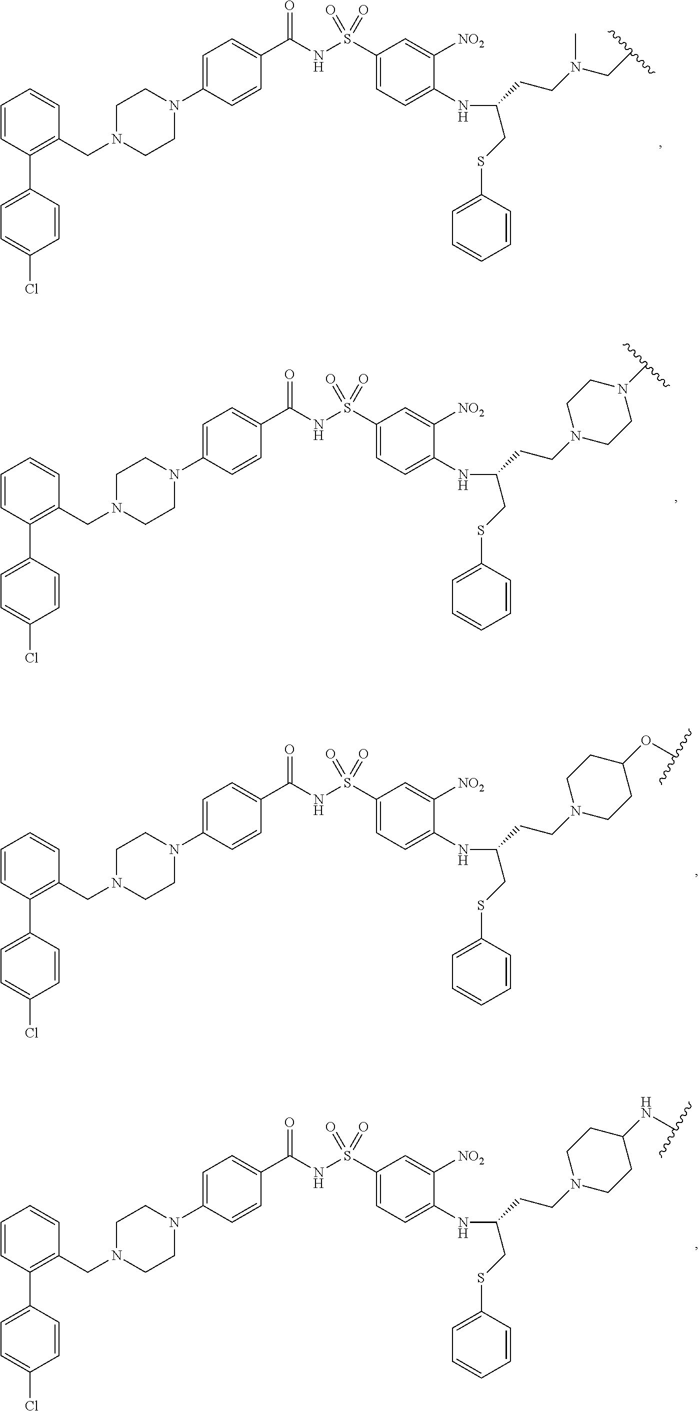 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

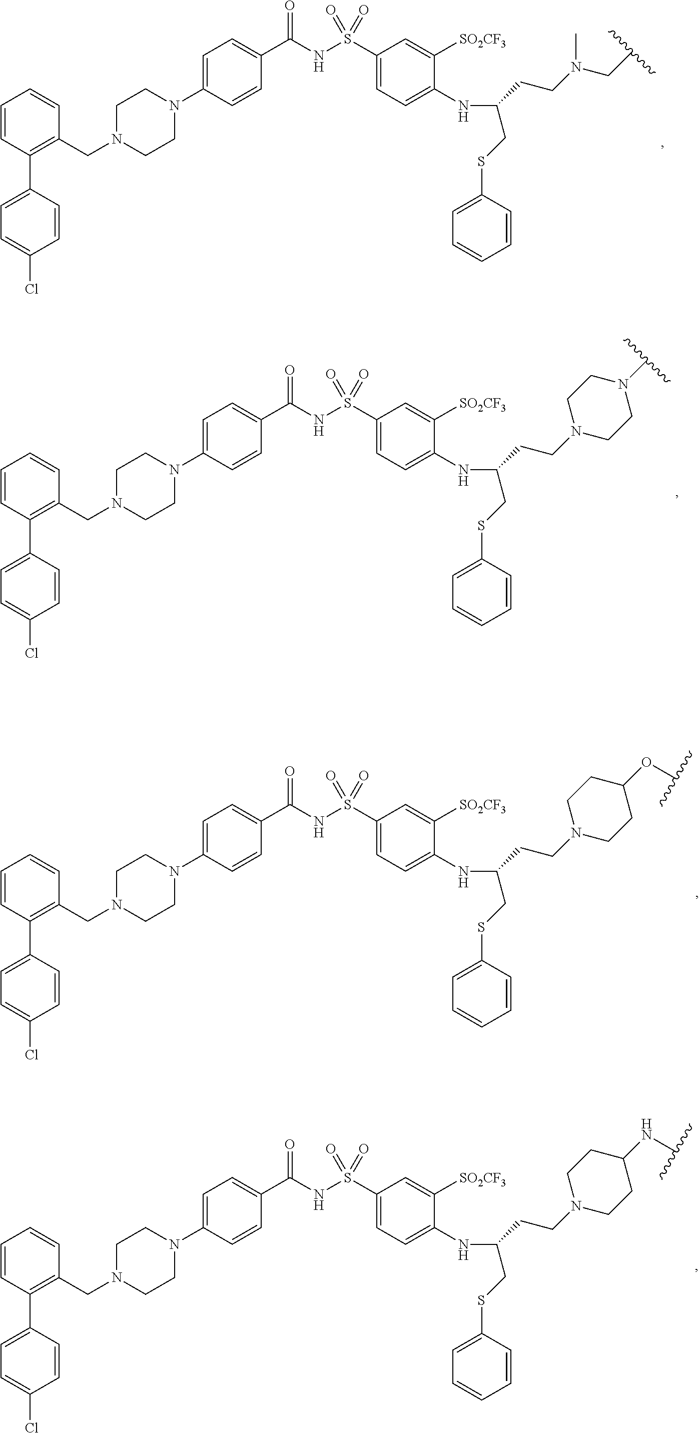

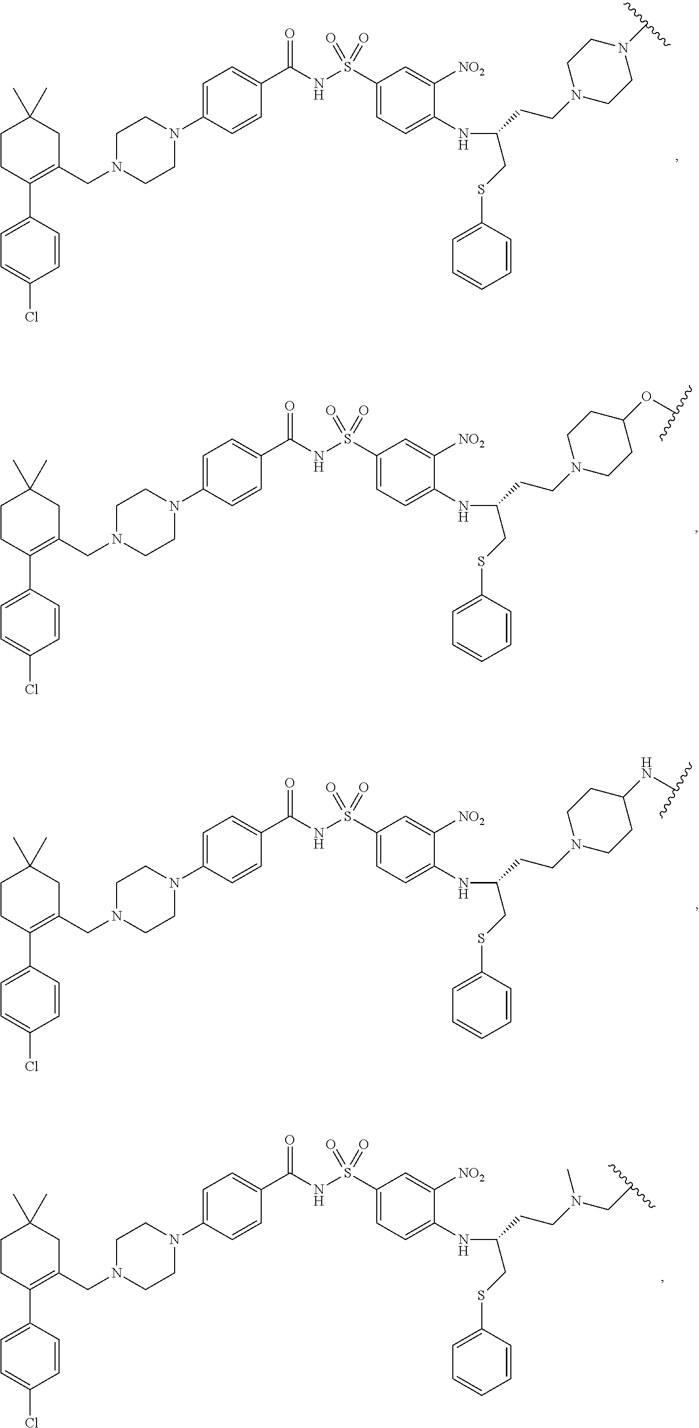 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

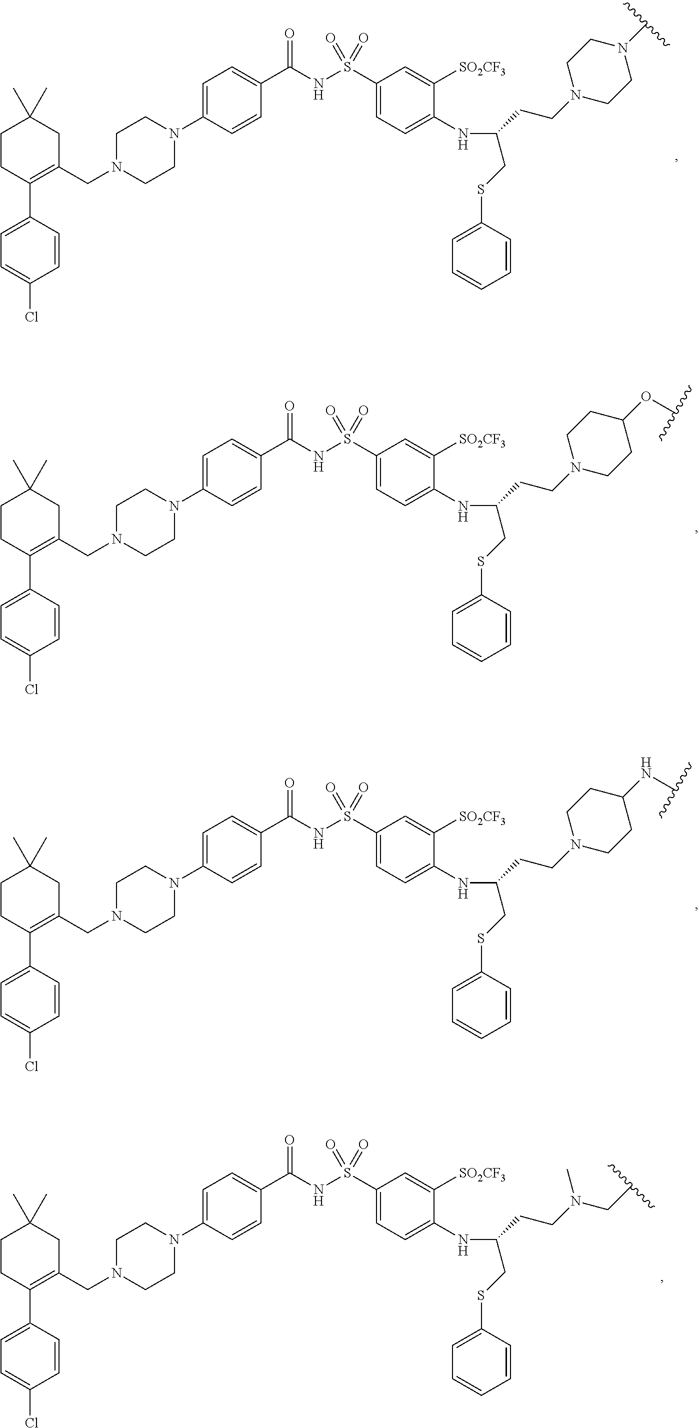

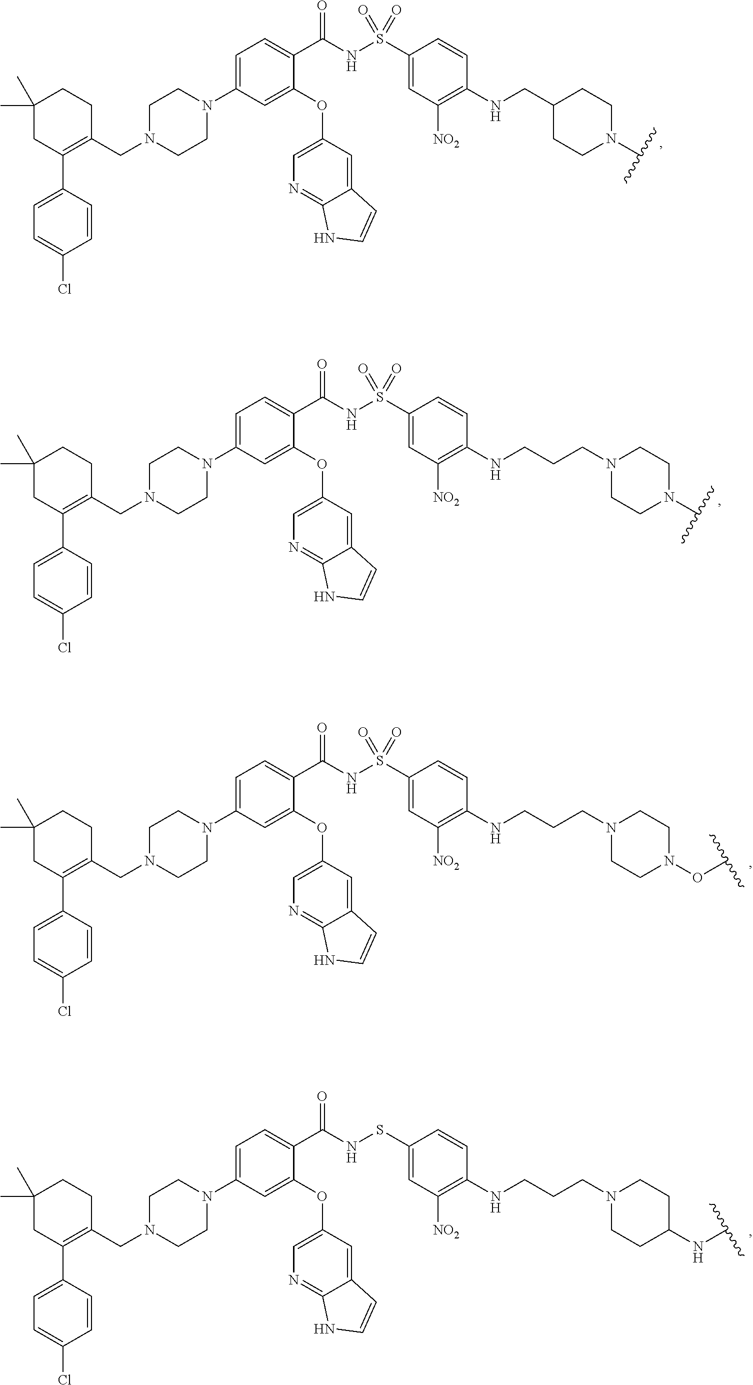 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *ON1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *NC1CCN(CCCNc2ccc(SNC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

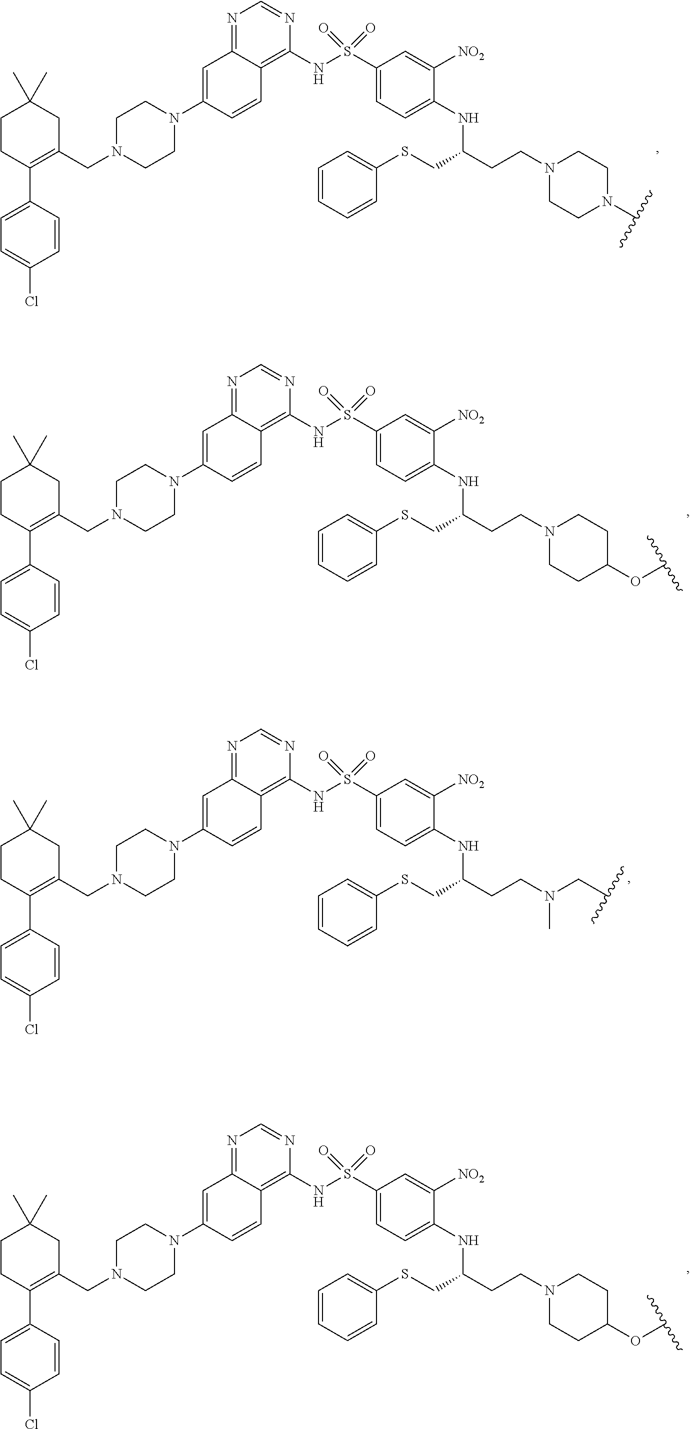 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

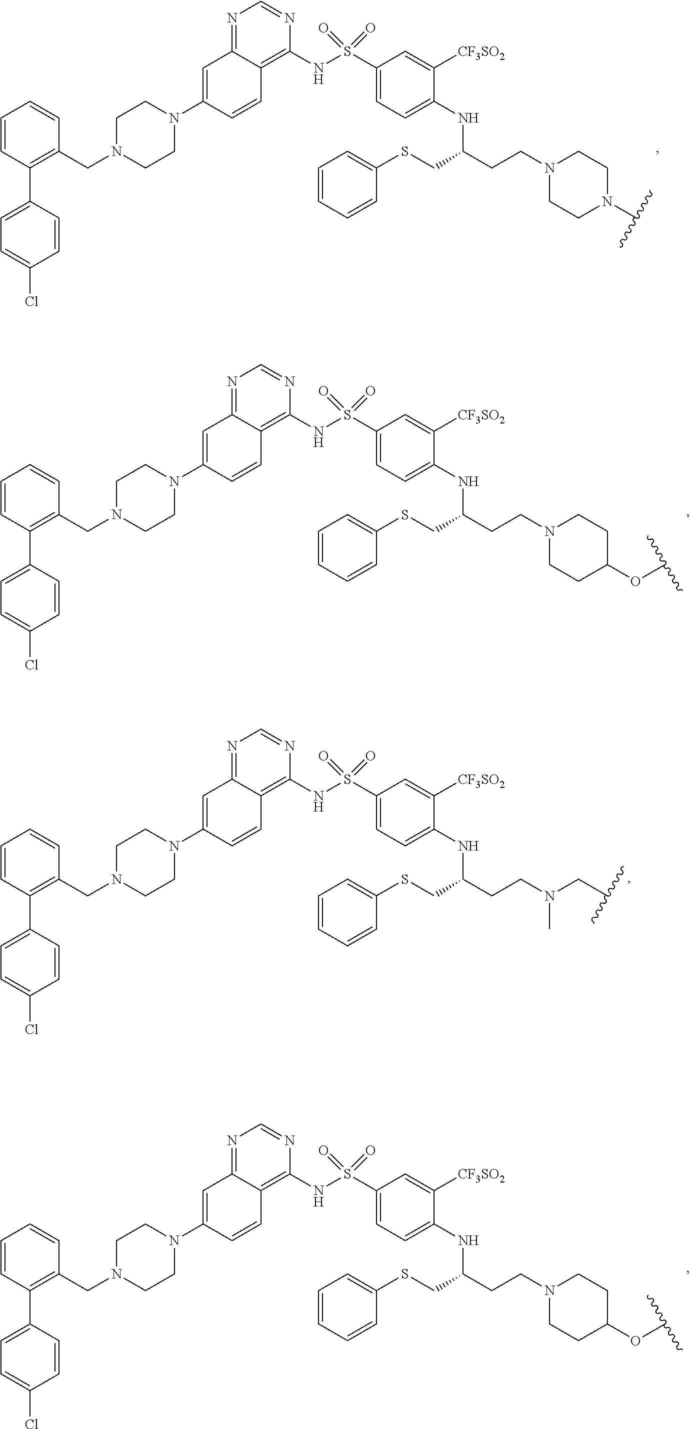

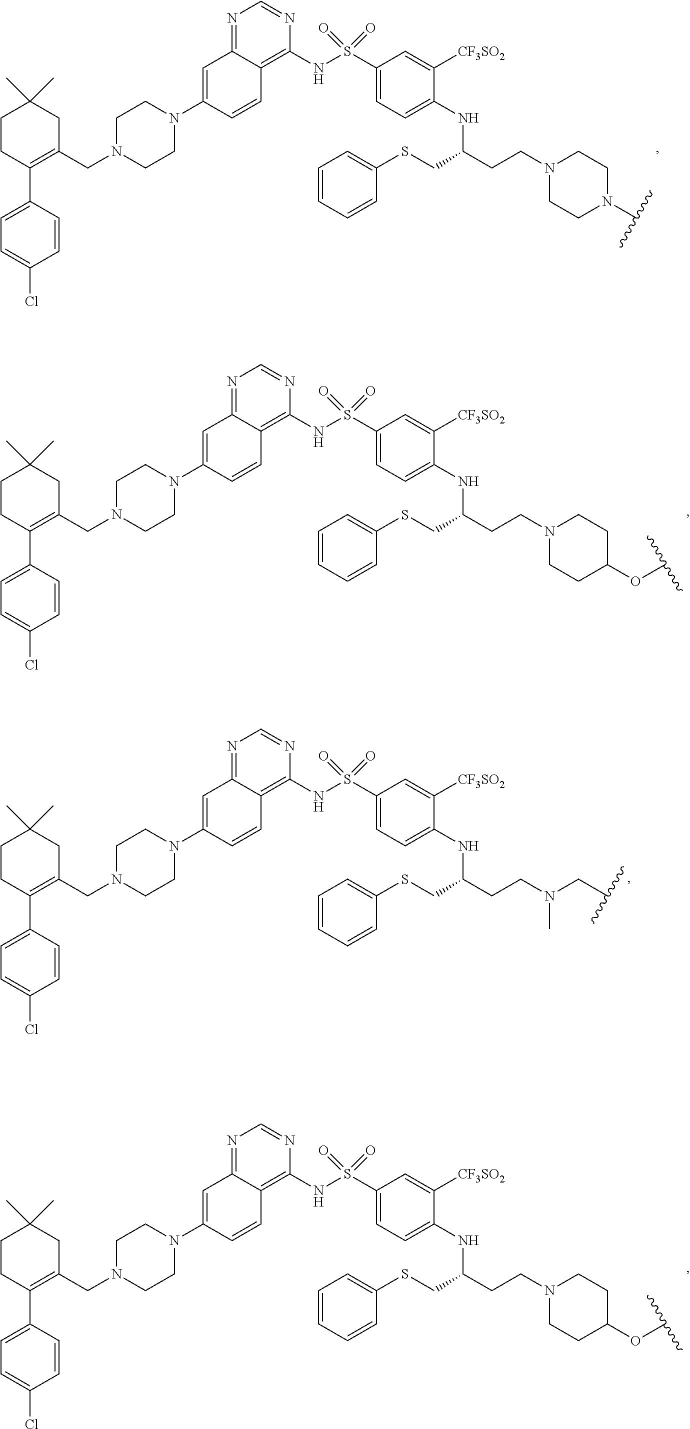

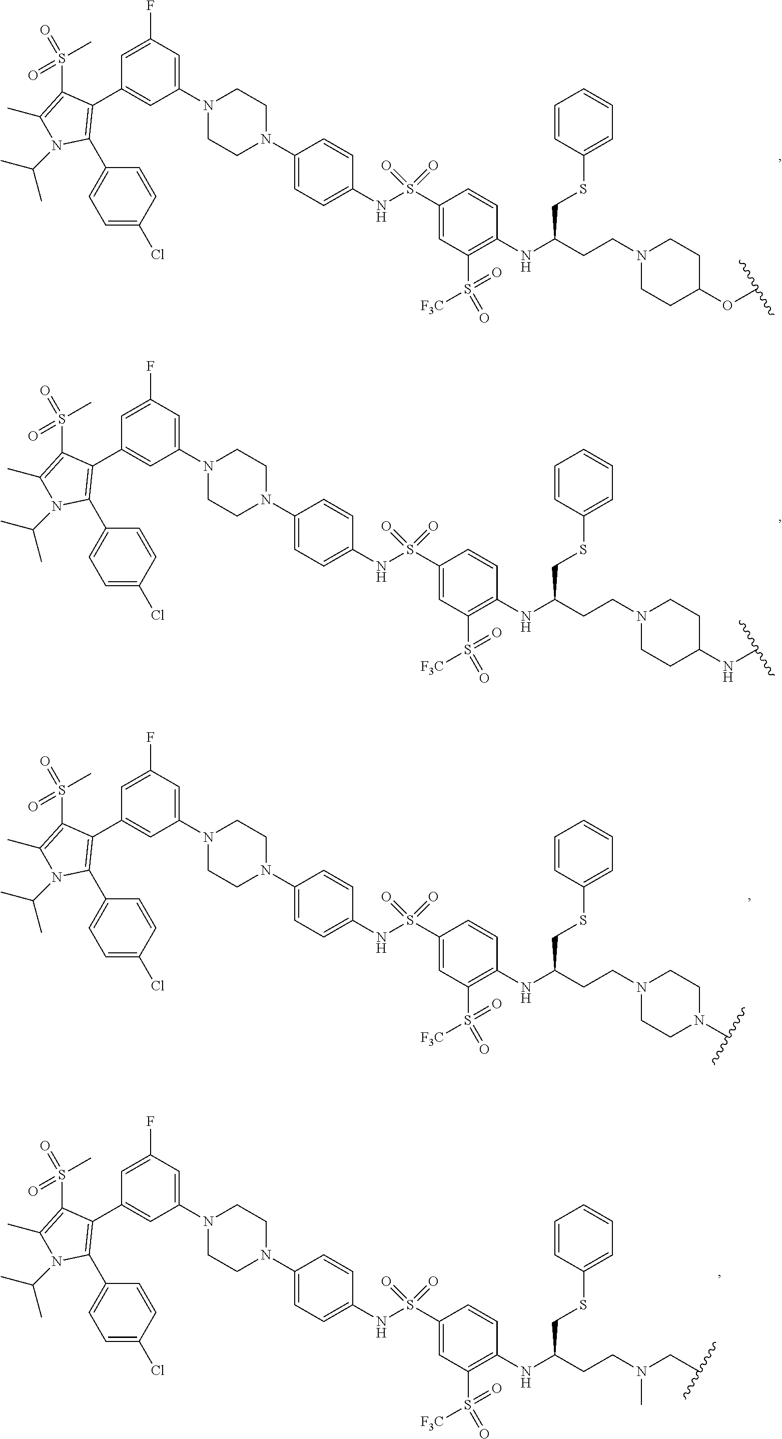

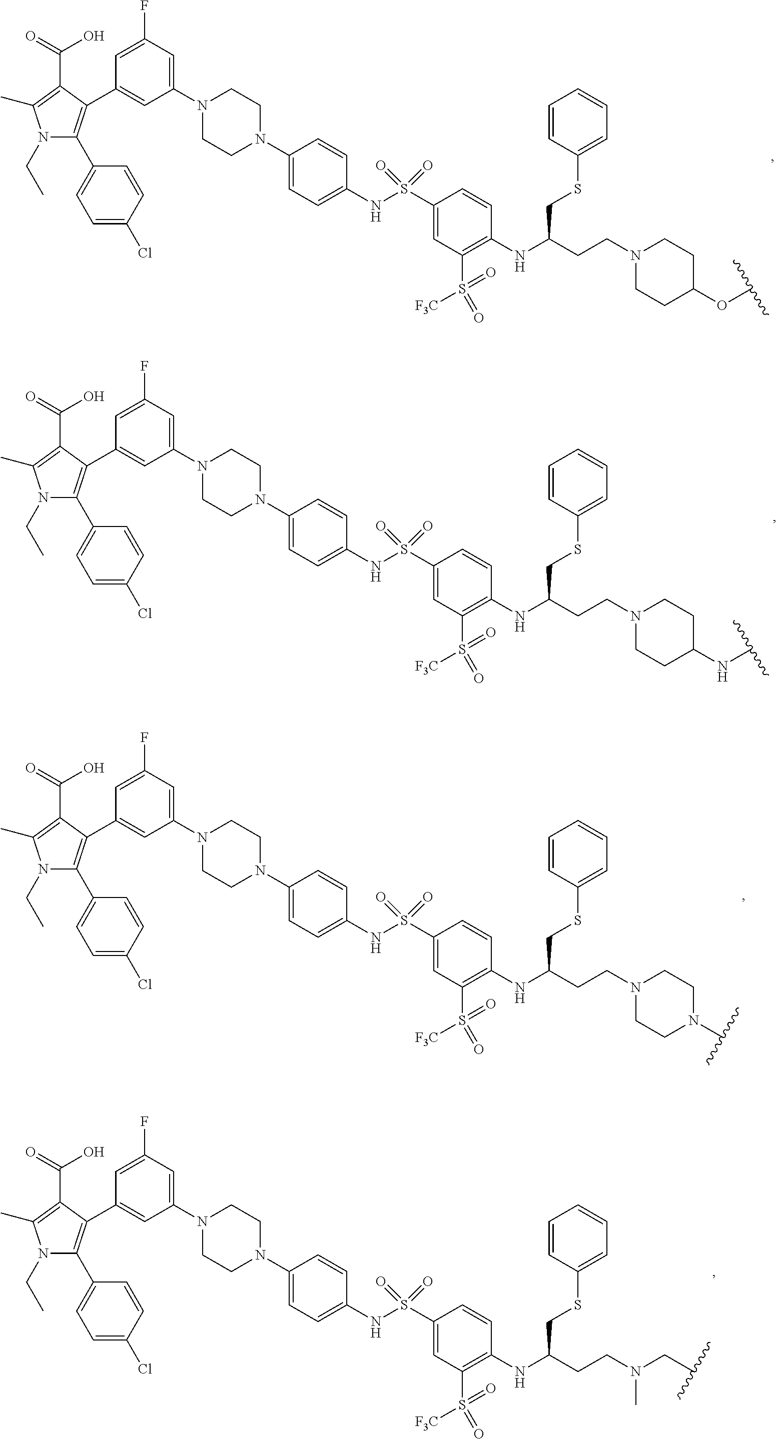

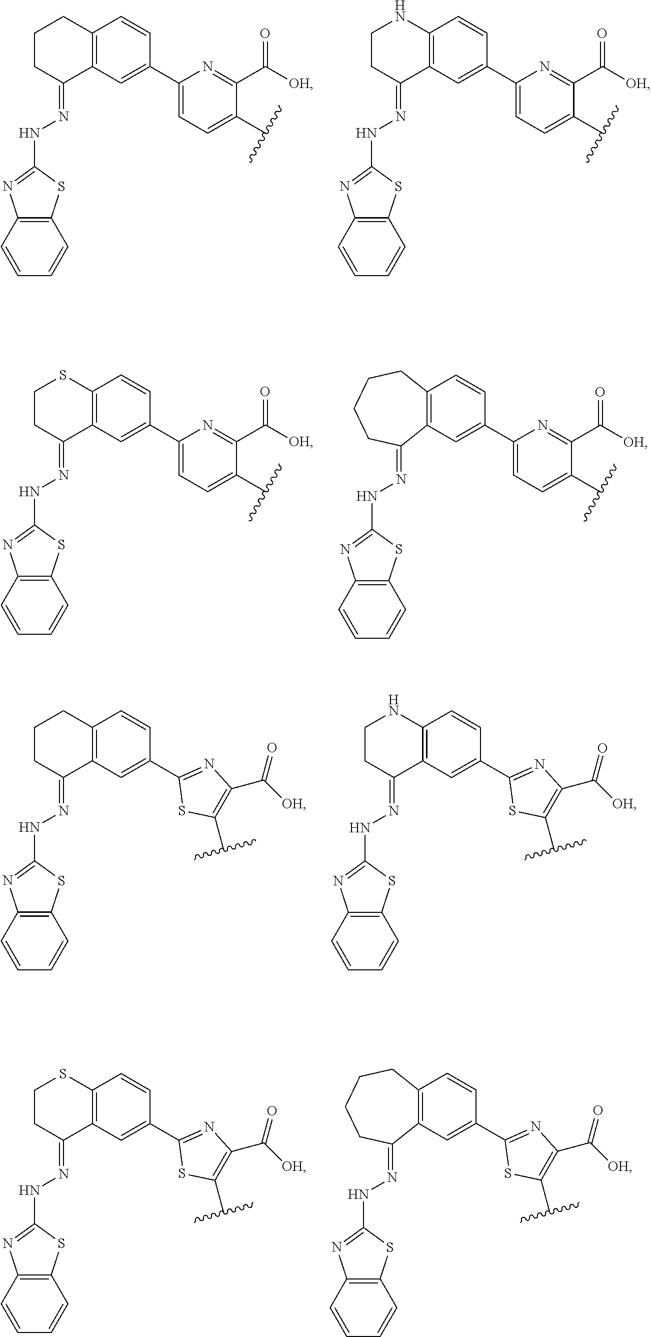

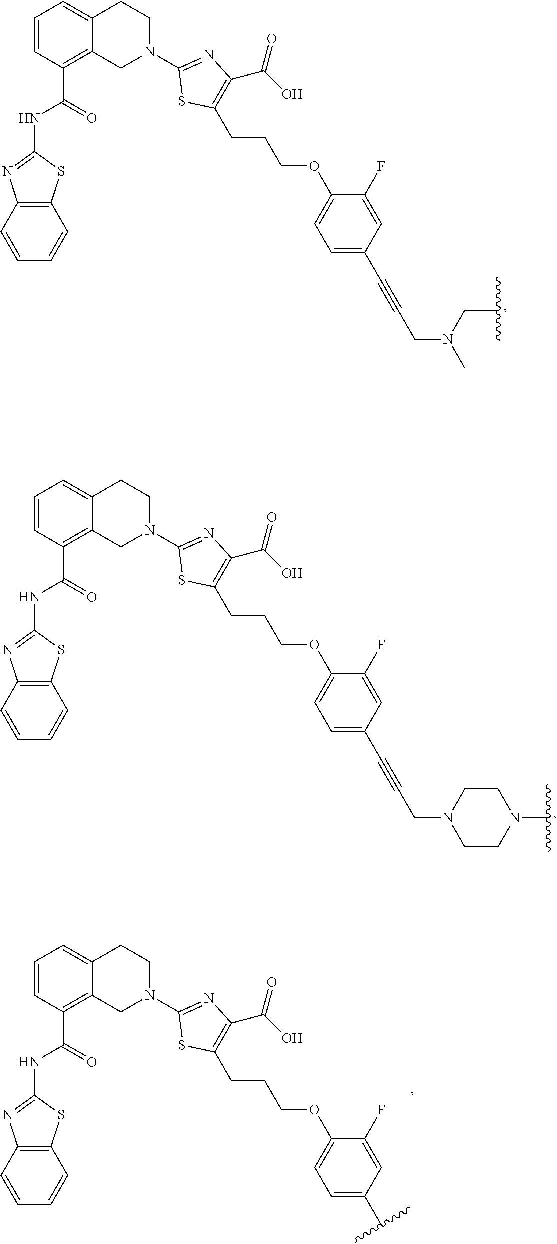 [SMILES: *CN(C)CC#Cc1ccc(OCCCc2sc(N3CCc4cccc(C(=O)Nc5nc6ccccc6s5)c4C3)nc2C(=O)O)c(F)c1 ; *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1]

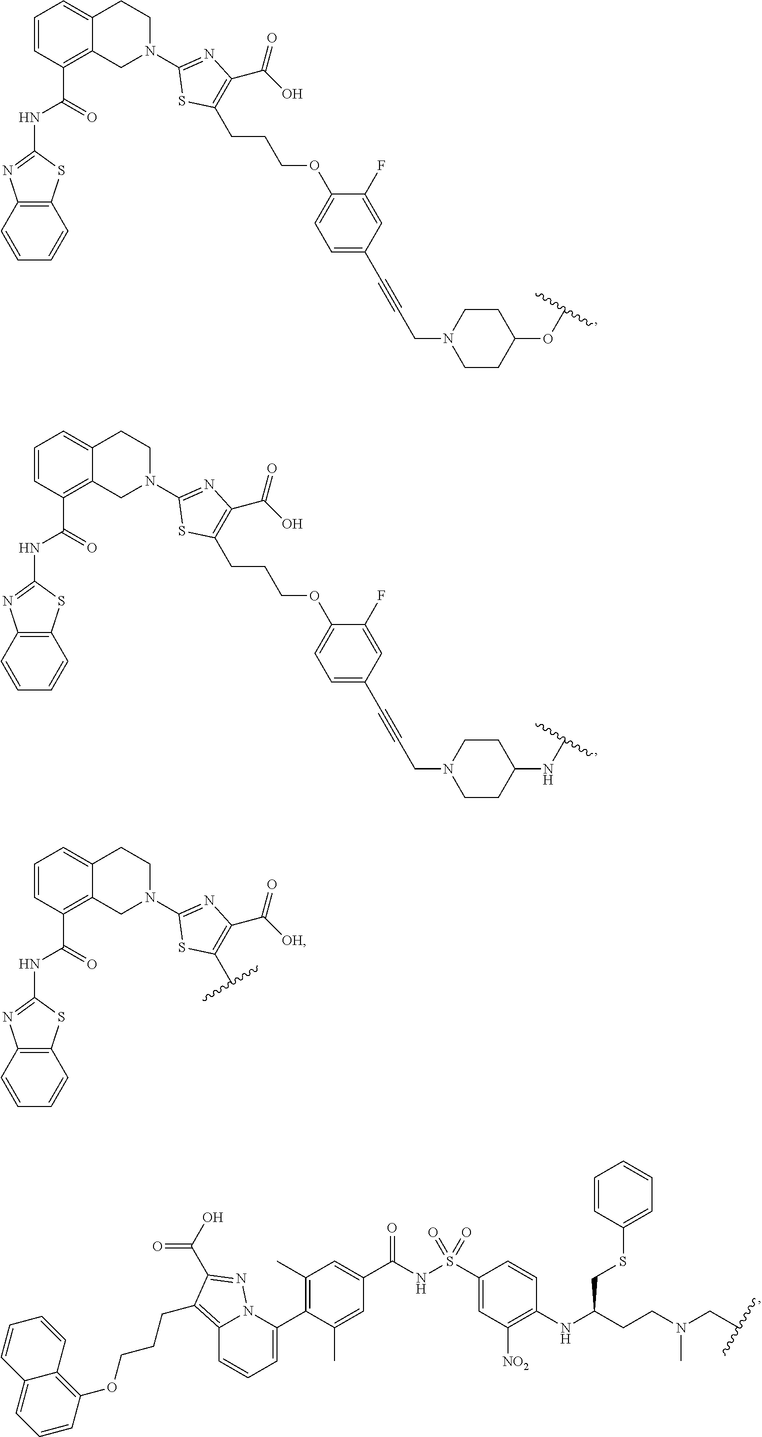 [SMILES: *OC1CCN(CC#Cc2ccc(OCCCc3sc(N4CCc5cccc(C(=O)Nc6nc7ccccc7s6)c5C4)nc3C(=O)O)c(F)c2)CC1 ; *NC1CCN(CC#Cc2ccc(OCCCc3sc(N4CCc5cccc(C(=O)Nc6nc7ccccc7s6)c5C4)nc3C(=O)O)c(F)c2)CC1 ; *c1sc(N2CCc3cccc(C(=O)Nc4nc5ccccc5s4)c3C2)nc1C(=O)O ; *CN(C)CC[C@H](CSc1ccccc1)Nc1ccc(S(=O)(=O)NC(=O)c2cc(C)c(-c3cccc4c(CCCOc5cccc6ccccc56)c(C(=O)O)nn34)c(C)c2)cc1[N+](=O)[O-]]

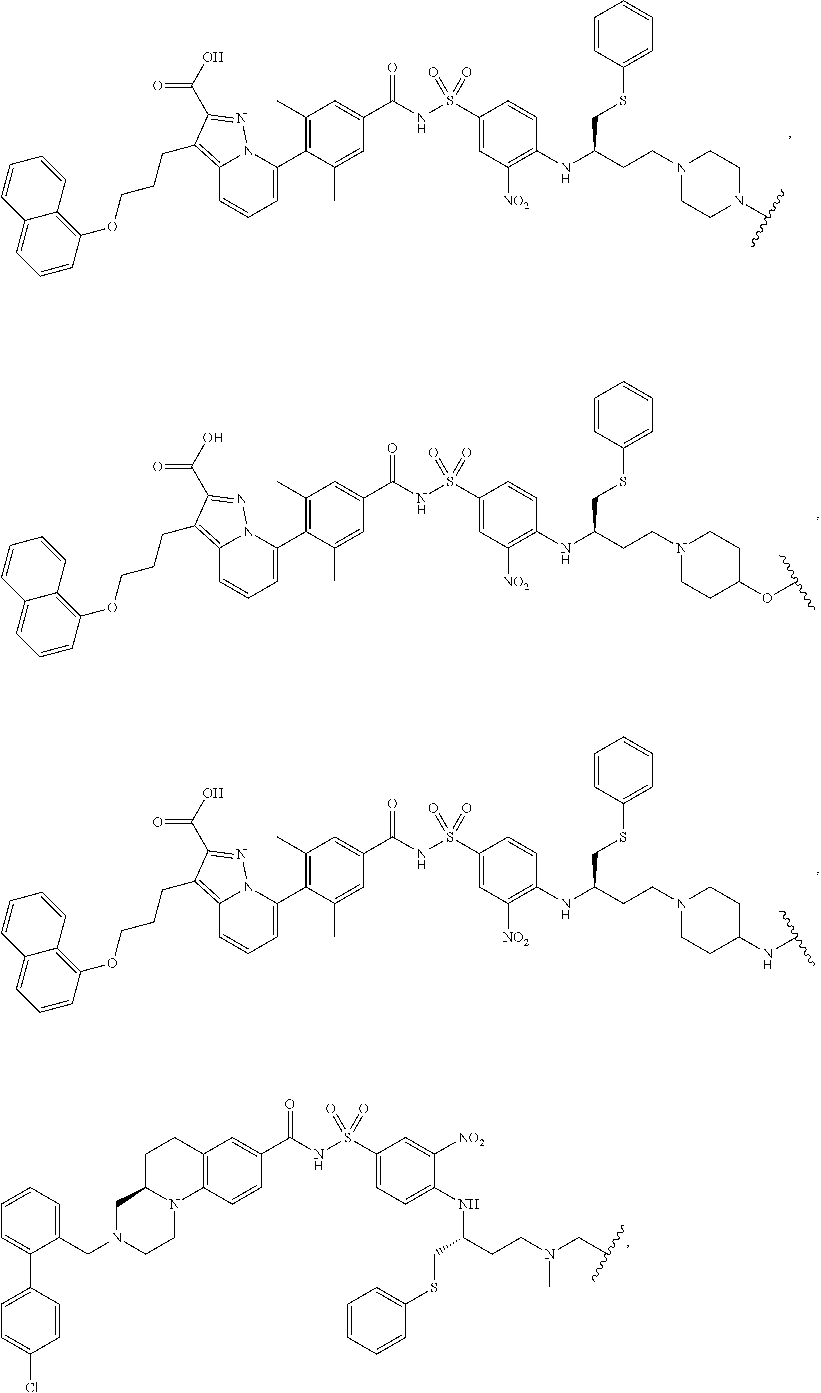

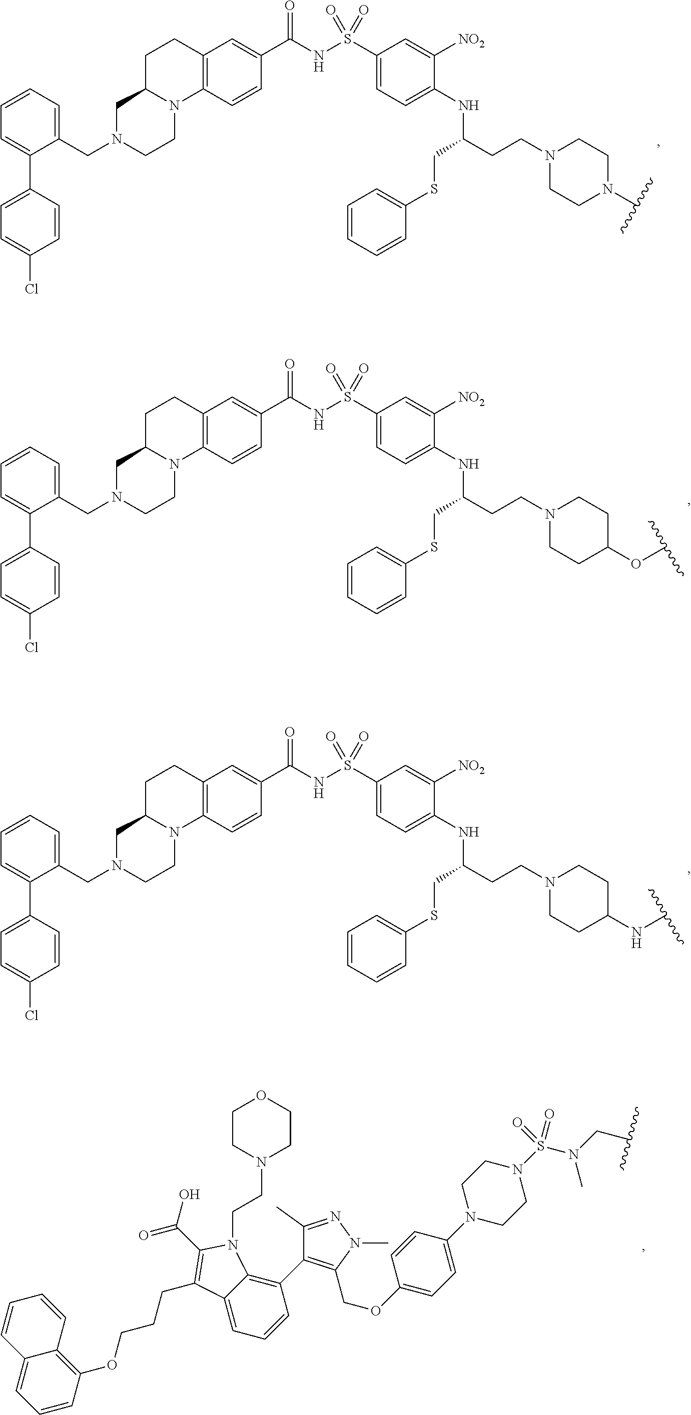 [SMILES: *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1]

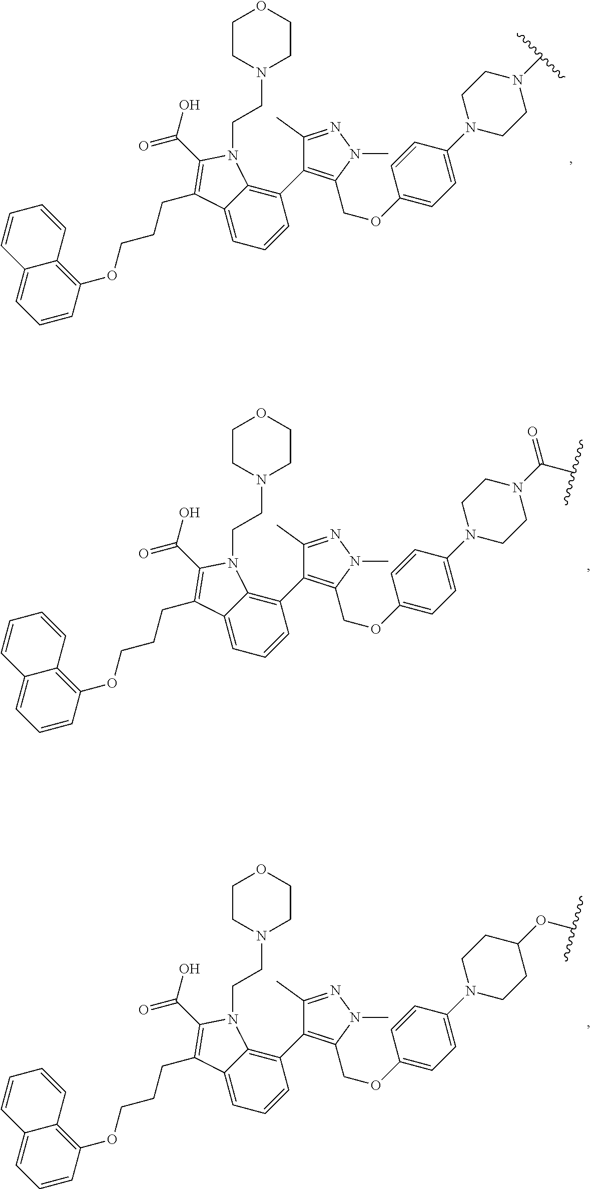 [SMILES: *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *OC1CCN(c2ccc(OCc3c(-c4cccc5c(CCCOc6cccc7ccccc67)c(C(=O)O)n(CCN6CCOCC6)c45)c(C)nn3C)cc2)CC1]

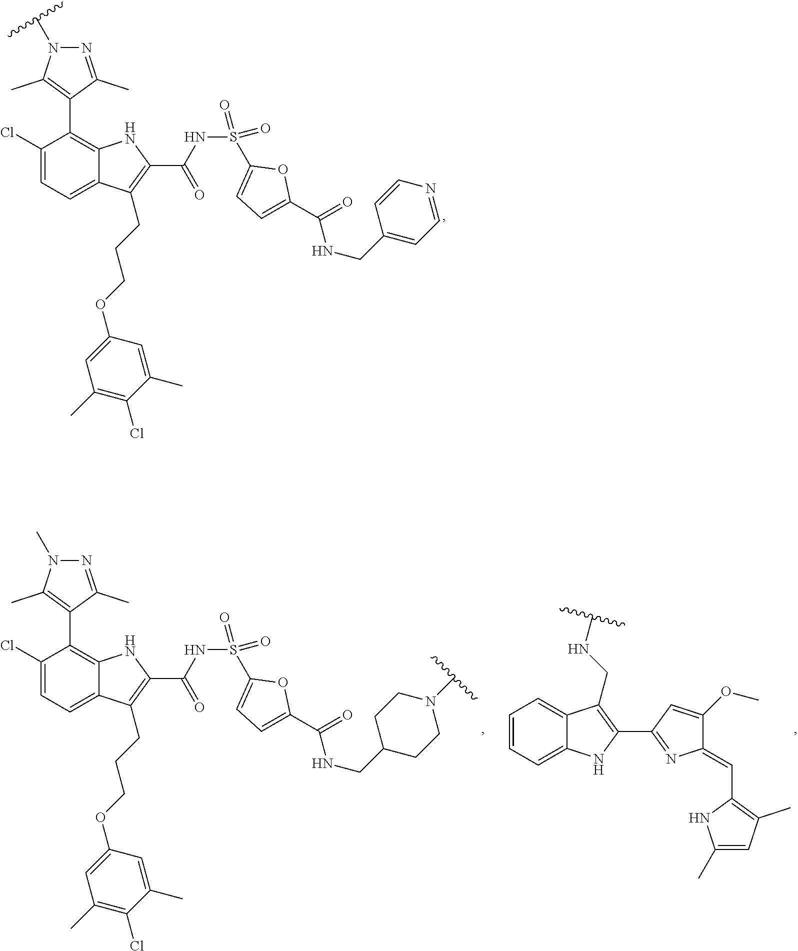 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12]

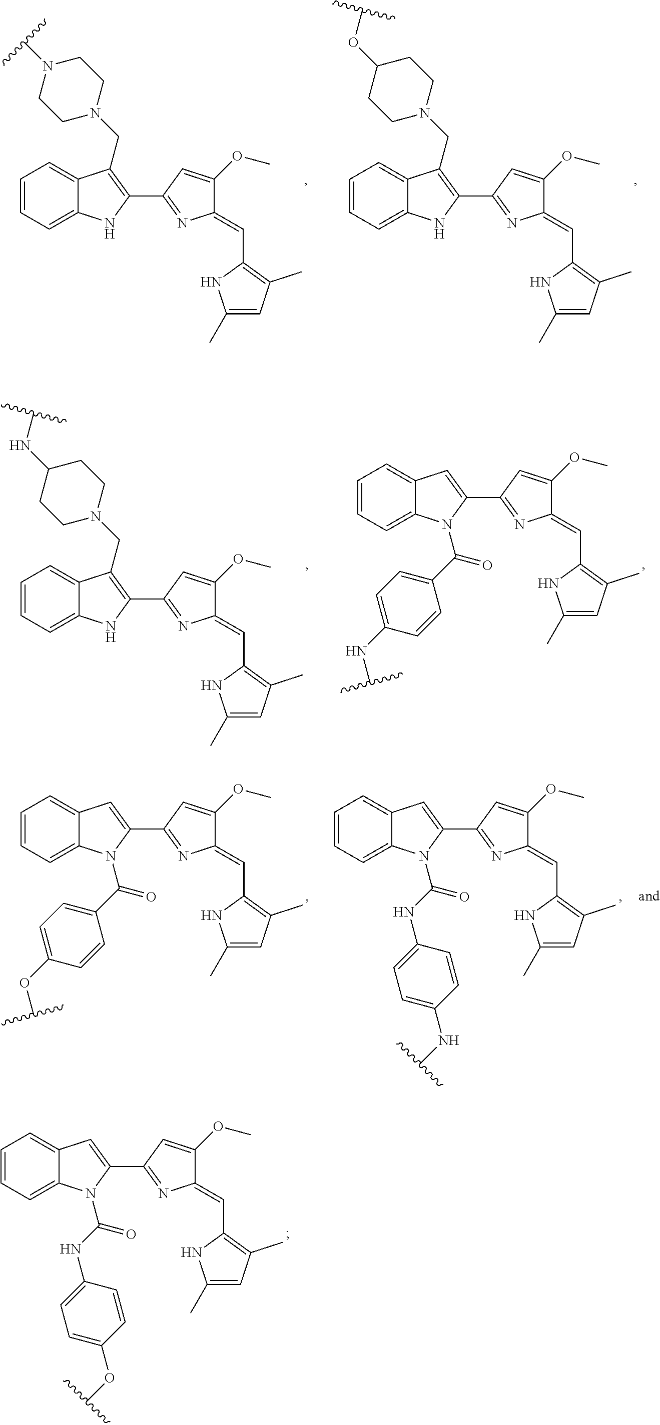

R³ is absent, a bond, or a substituted or unsubstituted C₁-C₁₀ alkyl;

A is absent, a bond, a substituted or unsubstituted C₁-C₆ aryl, a substituted or unsubstituted C₁-C₆ cycloalkyl, or a substituted or unsubstituted C₁-C₆ heterocyclic group;

R⁴ is a bond or a substituted or unsubstituted C₁-C₁₀ alky;

n is an integer from 0 to 5;

R² is selected from the group consisting of

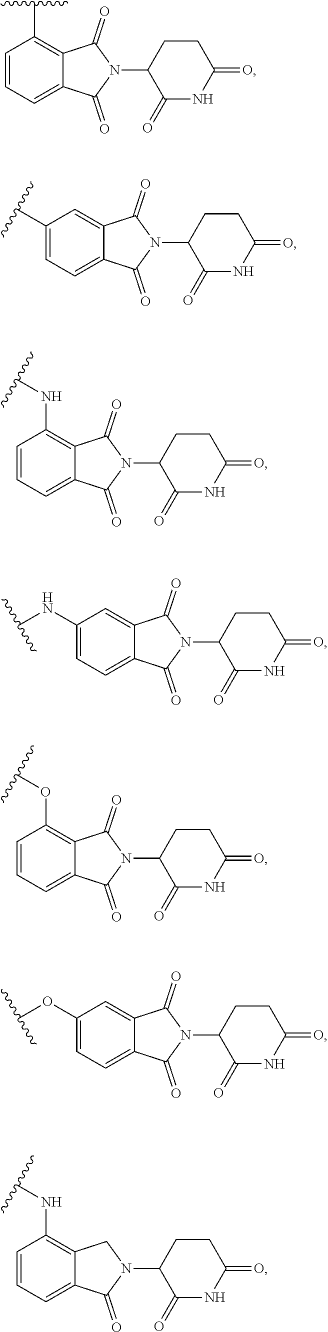

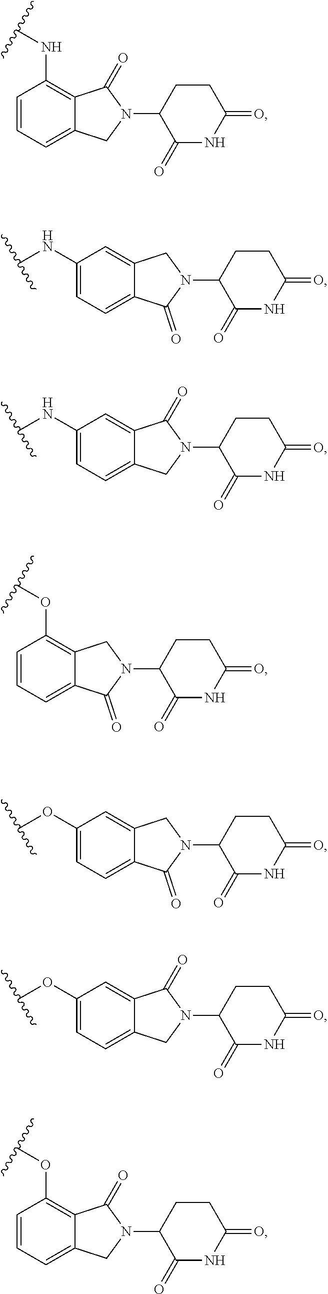

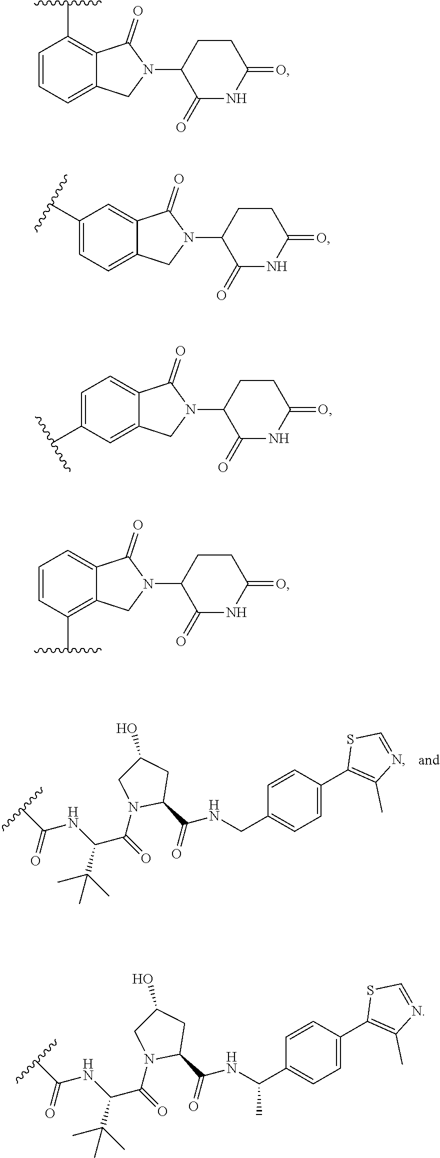

In another aspect, the invention encompasses a method for delaying at least one feature of aging in a subject. The method comprises administering a therapeutically effective amount of a compound of the invention to a subject in need thereof.

In yet another aspect, the invention encompasses a method of treating an age-related disease or condition. The method comprises administering a therapeutically effective amount of a compound of the invention to a subject in need thereof.

In still yet another aspect, the invention encompasses a method of killing therapy-induced senescent cells. The method comprises administering a therapeutically effective amount of a compound of the invention to a subject in need thereof who has received DNA-damaging therapy and killing therapy induced-senescent cells in normal and tumor tissues following DNA-damaging therapy.

### BRIEF DESCRIPTION OF THE FIGURES

FIG. 1A and FIG. 1B depicts graphs that show XZ-13906 (2 μM) depletes Bcl-xL in normal WI38 (NC-WI38) and ionizing radiation induced senescent WI38 (IR-SC WI38 cells).

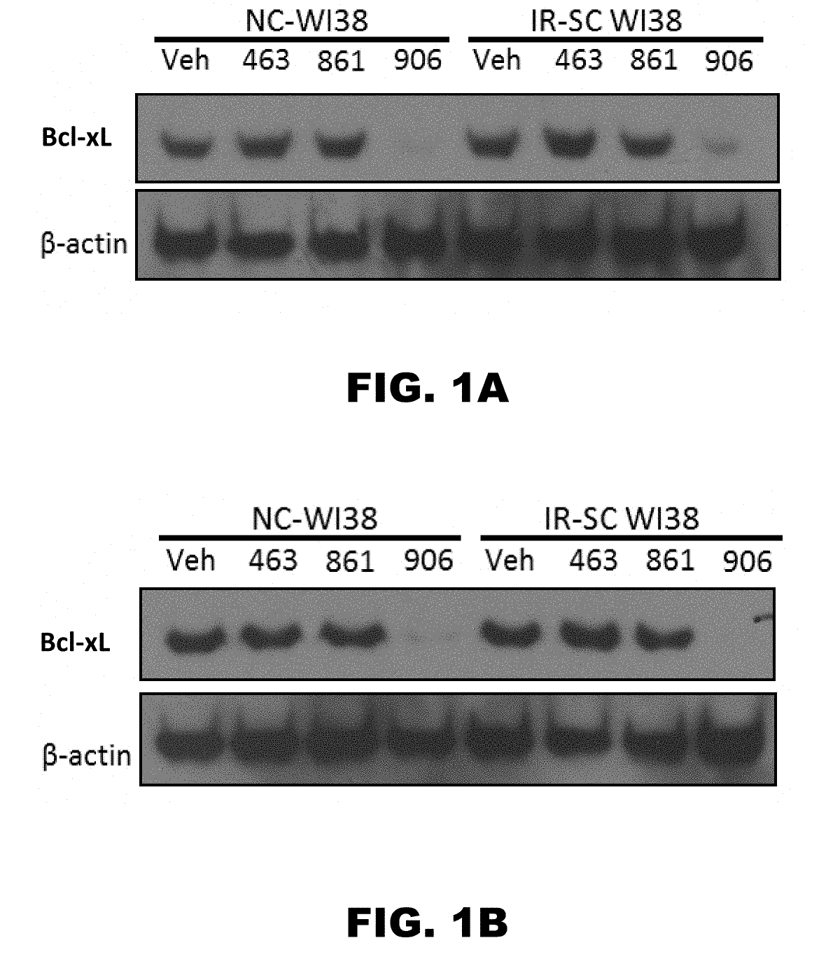

FIG. 2A and FIG. 2B depicts graphs show that compound 11 (XZ-13861) (FIG. 2A) and XZ-13906 (FIG. 2B) selectively inhibits IR-SC WI38 cells but not normal WI38 cells in a dose-dependent manner.

FIG. 3A and FIG. 3B depicts graphs that show that XZ-14439 dose dependent (FIG. 3A) and time dependently (FIG. 3B) depletes Bcl-xL in IR-SC WI38 cells.

FIG. 4A and FIG. 4B depicts graphs that show that XZ-15416, XZ-15405, XZ-15418, XZ-15421, and PZ-15227 deplete Bcl-xL in IR-SC WI38 (FIG. 4A) and RS4; 11 (FIG. 4B) cells.

FIG. 5 shows the clearance of senescent cells with ABT263 reversed Cisplatin-induced peripheral neuropathy (CIPN) in C57BL/6 mice. CIPN was induced in young adult male and female C57BL/6 mice by intraperitoneal (i.p.) injection of Cisplatin (Lake Zurich, Ill., USA) at 2.3 mg/kg/d for 5 days per cycle for 2 cycles with an interval of 5 days between the cycles. The induction of CIPN was measured by analyzing the mechanical sensitivity using the Von Frey assay the day before Cisplatin treatment and on various day after Cisplatin treatment

FIG. 6 depicts the data from FIG. 5 in a bar graph to show statistical differences among the different treatment groups. N=10 for control group and 5 for Cisplatin-treated groups. ** and ***p<0.01 and 0.001, respectively, vs. Control.

FIG. 7 shows the clearance of senescent cells with ABT263 reversed cisplatin-induced peripheral neuropathy (CIPN) in C57BL/6 mice. Mice were treated as described in FIG. 1. The induction of CIPN was measured by analyzing the sensitivity to thermal allodynia using the Hot Plate Analgesia Meter on various day after Cisplatin treatment. N=10 for control group and 5 for Cisplatin-treated groups. ***p<0.001 vs. Control.

FIG. 8 shows both genetic and pharmacological clearance of senescent cells with ganciclovir (GCV) and ABT263, respectively, reversed cisplatin-induced peripheral neuropathy (CIPN) in p16-3MR transgenic mice. CIPN was induced in young adult male and female p16-3MR mice by intraperitoneal (i.p.) injection of cisplatin (Lake Zurich, Ill., USA) at 2.3 mg/kg/d for 5 days per cycle for 2 cycles with an interval of 5 days between the cycles. The induction of CIPN was measured by analyzing the mechanical sensitivity using the Von Frey assay the day before Cisplatin treatment and on various day after Cisplatin treatment. N=5-6 mice per group.

FIG. 9 depicts data from FIG. 8 in a bar graph to show statistical differences among the different treatment groups. N=5-6 mice per group. *, ** and ***p<0.05, 0.01, and 0.001, respectively, vs. Saline or Saline+GCV group.

FIG. 10 shows both genetic and pharmacological clearance of senescent cells with ganciclovir (GCV) and ABT263, respectively, reversed cisplatin-induced peripheral neuropathy (CIPN) in p16-3MR transgenic mice. Mice were treated as described in FIG. 8. The induction of CIPN was measured by analyzing the sensitivity to thermal allodynia using the Hot Plate Analgesia Meter on various day after Cisplatin treatment. N=5-6 mice per group.

FIG. 11 depicts the data from FIG. 10 in a bar graph to show statistical differences among the different treatment groups. N=5-6 mice per group. *, ** and ***p<0.05, 0.01, and 0.001, respectively, vs. Saline or Saline+GCV group.

FIG. 12A and FIG. 12B show five-day administration of ABT-263 or Bcl-PROTAC eliminates senescent osteocytes in aged mice. FIG. 12A shows a western blot analysis of markers of DNA damage (γ-H2AX) and cellular senescence (GATA4 and p16), each lane represents one animal. FIG. 12B depicts mRNA levels by qPCR analysis in femoral extracts (n=4/group), *p<0.05.

FIG. 13 shows five-day administration of ABT-263 or Bcl-PROTAC eliminates osteoprogenitor senescence and SASP in aged mice. mRNA levels by qRT-PCR in bone marrow stromal cells from 24-month-old female mice cultured with ascorbate and β-glycerophosphate for 7 days (triplicate cultures), *p<0.05.

FIG. 14A, FIG. 14B and FIG. 14C show five-day administration of ABT-263 or Bcl-PROTAC to mice promotes osteoblastogenesis. FIG. 14A shows Alizarin Red staining and FIG. 14B depicts quantification in stromal cells cultured with ascorbate and b-glycerophosphate for 21 d. FIG. 14C shows the quantification of Oil Red 0 staining in stromal cells cultured with rosiglitazone for 11 d. Triplicate cultures; *p<0.05

FIG. 15A and FIG. 15B show five-day administration of ABT-263 or Bcl-PROTAC to mice decreases osteoclast progenitor number. FIG. 15A depicts representative pictures of TRAP-positive multinucleated cells generated from bone marrow macrophages.

FIG. 15B show TRAP-positive multinucleated cells containing three or more nuclei were counted as osteoclasts (triplicate cultures). *p<0.05.

### DETAILED DESCRIPTION OF THE INVENTION

In part, the present invention relates to compounds which are capable of degrading the Bcl-2 family of proteins. The bivalent compounds connect a Bcl-2 small molecule inhibitor or ligand to an E3 ligase binding moiety, such as cereblon (CRBN) E3 ligase binding moiety (thalidomide derivatives such as pomalidomide) or von Hippel-Landau (VHL) E3 ligase binding moiety (such as HIF-1α-derived (R)-hydroxyproline containing VHL E3 ligase ligands). CRBN is part of the cullin-4 (CUL4) containing E3 ubiquitin ligase complex CUL4-RBX1-DDB1-CRBN (known as CRL4CRBN. Thalidomide and its derivatives, such as lenalidomide and pomalidomide, interact specifically with this CRBN complex and inducing degradation of essential IKAROS transcription factors. VHL is part of the cullin-2 (CUL2) containing E3 ubiquitin ligase complex elongin BC-CUL2-VHL (known as CRL2VHL) responsible for degradation of the transcription factor HIF-1α. (R)-Hydroxyproline containing VHL E3 ligase ligands derived from HIF-1α have been identified with high affinity. The bivalent compounds can actively recruit the Bcl-2 family of proteins to an E3 ubiquitin ligase, such as CRBN or VHL E3 ligase, resulting in their degradation by ubiquitin proteasome system.

Applicants have discovered that compounds comprising a moiety that selectively binds to an E3 ubiquitin ligase and a moiety that selectively binds a target protein, results in ubiquitination and subsequent degradation of the target protein through the ubiquitin proteasome system. Accordingly, the present disclosure provides compositions and methods for selectively degrading the Bcl-2 family of proteins.

Senescent cells (SCs) can cause chronic inflammation and oxidative stress through expression of senescence-associated secretory phenotype (SASP) and production of reactive oxygen species (ROS). The expression of SASP and production of ROS may contribute to CIPN. As such, clearance of SCs with a senolytic agent that can selectively kill SCs provides a novel therapeutic strategy to prevent, mitigate and treat CIPN. The present invention is directed to a method for the treatment of chemotherapy induced peripheral neuropathy comprising the step of administering to a subject in need thereof a therapeutically-effective amount of a small molecule senolytic agent that selectively kills senescent cells over non-senescent cells. As described in greater detail herein, senolytic agents include, but are not limited to, MDM2 inhibitors (e.g., nutlin 3α, RG-7112); inhibitors of one or more BCL-2 anti-apoptotic protein family members, which inhibitors inhibit a function of at least the anti-apoptotic protein, BCL-xL (e.g., ABT-263, ABT-737, WEHI-539, A-1155463); and Akt specific inhibitors (e.g., MK-2206). Senolytic agents described herein are sufficient to kill significant numbers of senescent cells.

Additional aspects of the invention are described below.

### I. Compositions

In an aspect, a composition of the invention comprises a compound of Formula (I) or a compound of Formula (II). Derivatives of Formula (I) or Formula (II) may be made to improve potency, bioavailability, solubility, stability, handling properties, or a combination thereof, as compared to an unmodified version.

A composition of the invention may optionally comprise one or more additional drugs or therapeutically active agents in addition to a compound of Formula (I) or a compound of Formula (II). A composition of the invention may further comprise a pharmaceutically acceptable excipient, carrier or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants.

### (a) Compounds of Formula (I)

Provide herein are compounds comprising Formula (I):

R₁-L-R₂ (I)

wherein

R₁ is a protein targeting unit which binds to one or more anti-apoptotic Bcl-2 family of proteins;

L is a linker unit which covalently links R₁ and R₂ through an alkyl, branched alkyl, ether, thioether, ester, amine, amide, carbamate, carbamide, sulfone, aryl, heteroaryl, cycloalkyl, or heterocyclic group, both end can be same or different; the linker unit could contain a combination of two or more groups among alkyl, branched alkyl, ether, thioether, ester, amine, amide, carbamate, carbamide, sulfone, aryl, heteroaryl, cycloalkyl, and heterocyclic groups; the linker unit comprises a length of 1-30 atoms in shortest length; and

R₂ is an E3 ubiquitin ligase binding unit which binds to the CRBN or VHL E3 ubiquitin ligase.

### (b) Compounds of Formula (II)

The compounds described by Formula (II) are a subset of the compounds described by Formula (I). Thus, R₁ and R₂ in Formula (I) are equivalent to R₁ and R₂ in Formula (II), respectively. The L in Formula (I) is defined as the following in Formula (II):

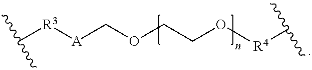

Also provided herein are compounds comprising Formula (II) or an isomer thereof:

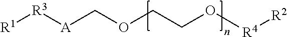

wherein

R¹ is selected from the group consisting of:

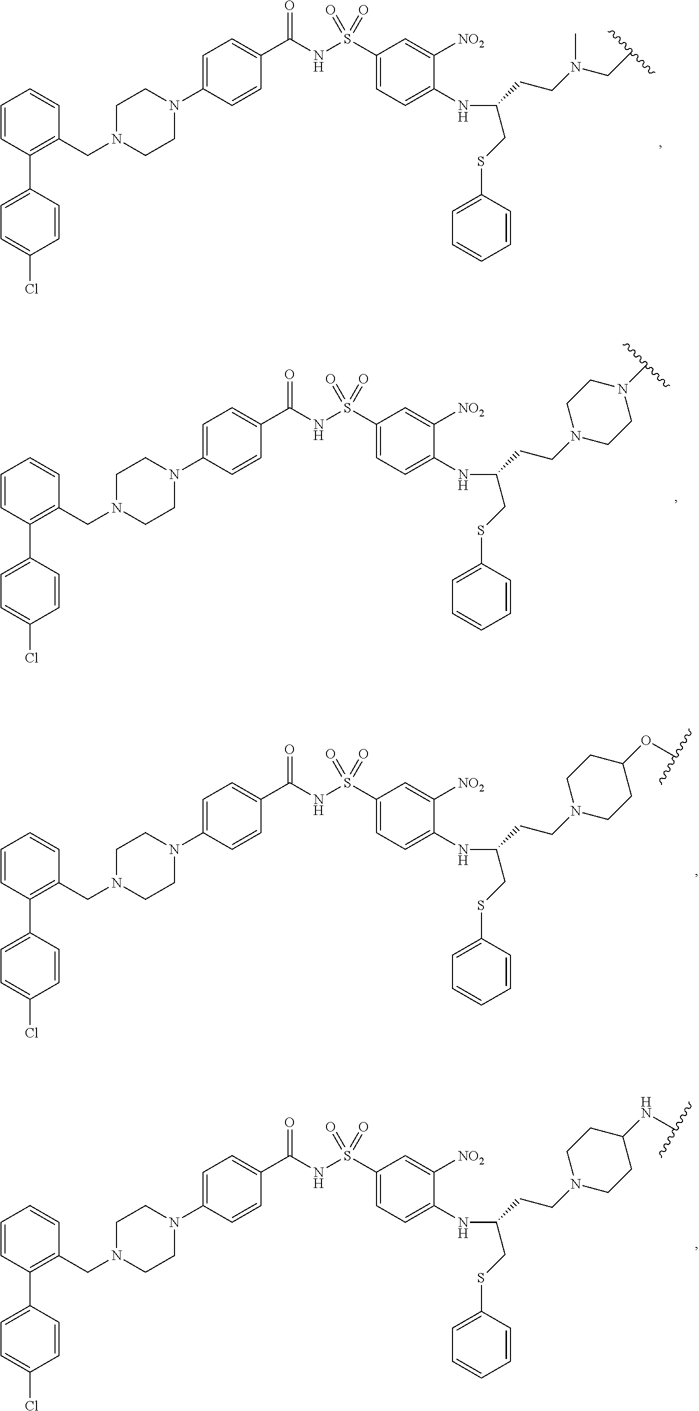 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

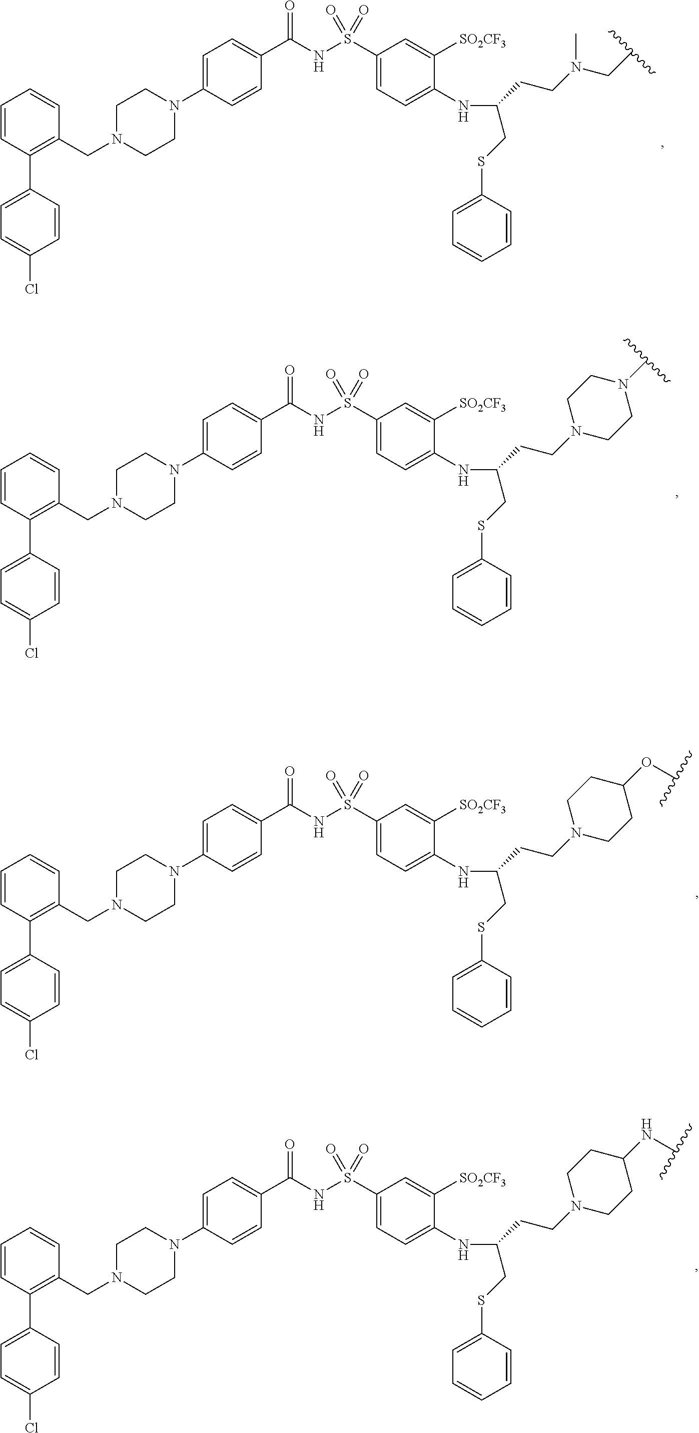

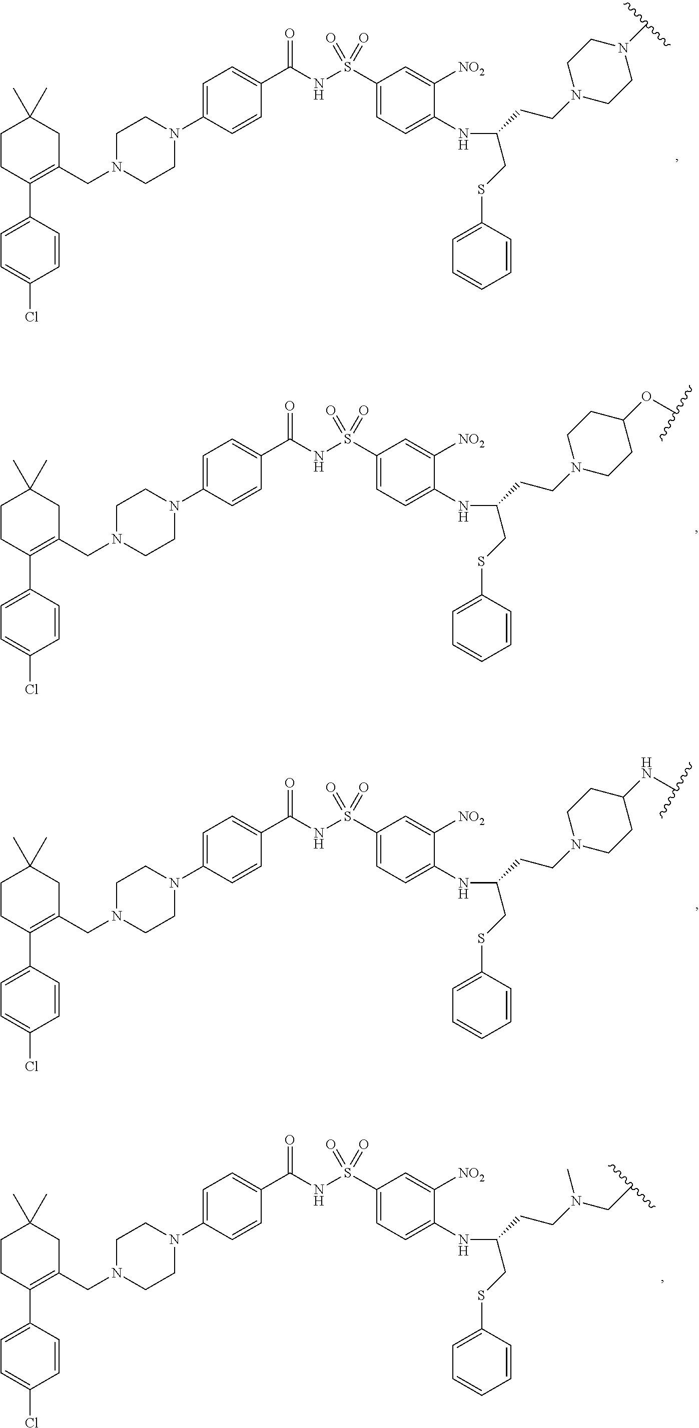 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

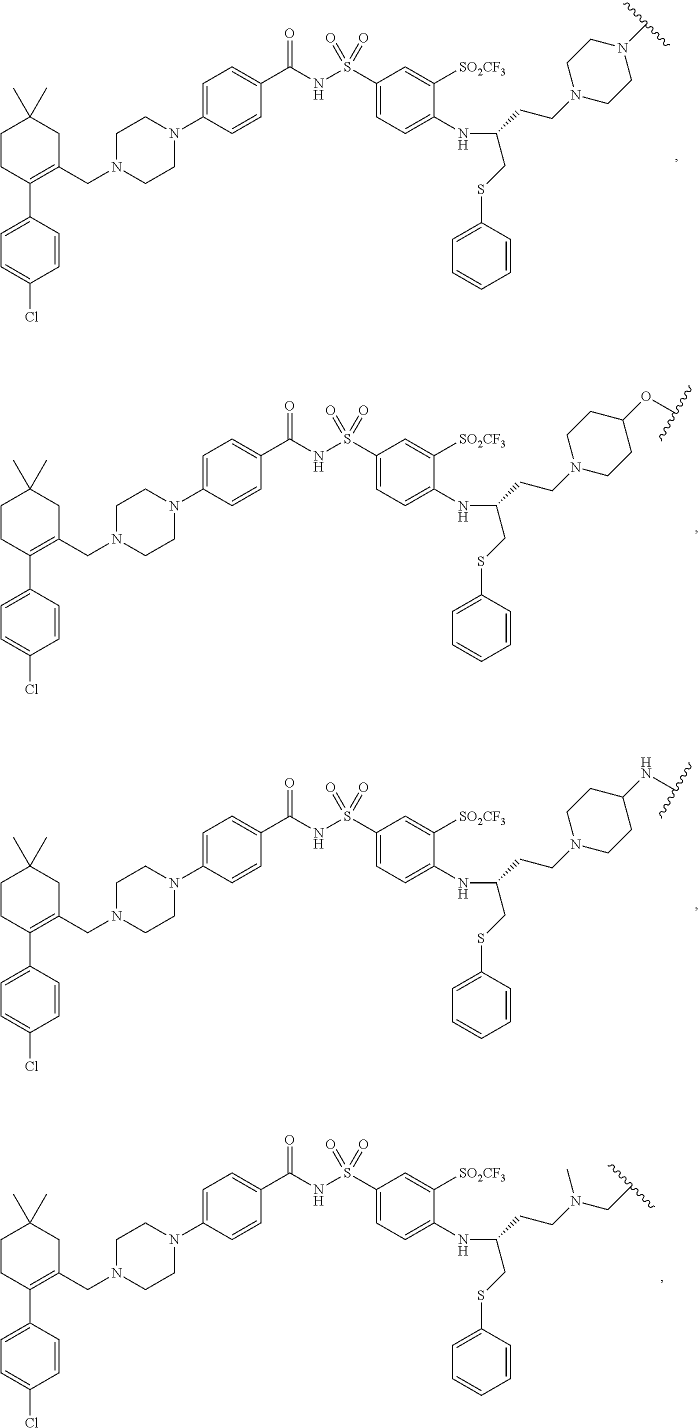

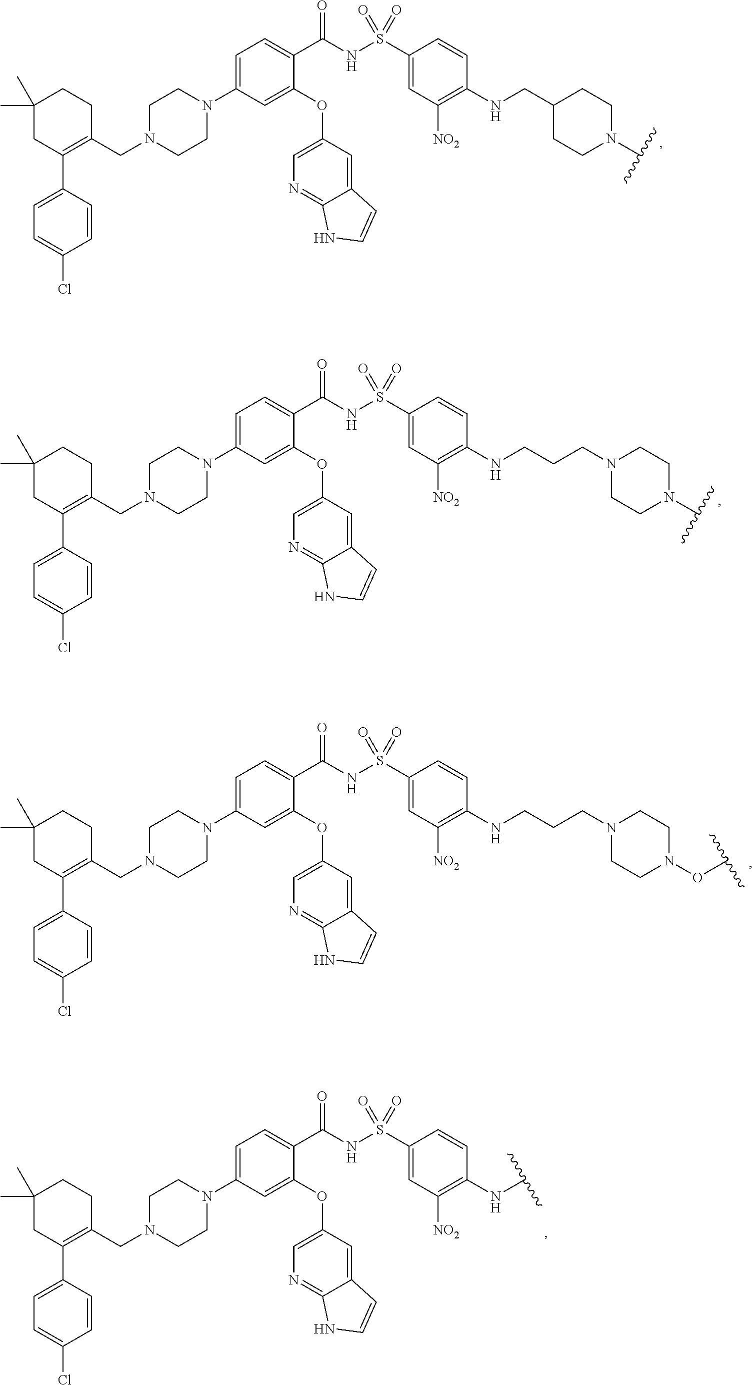 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *ON1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

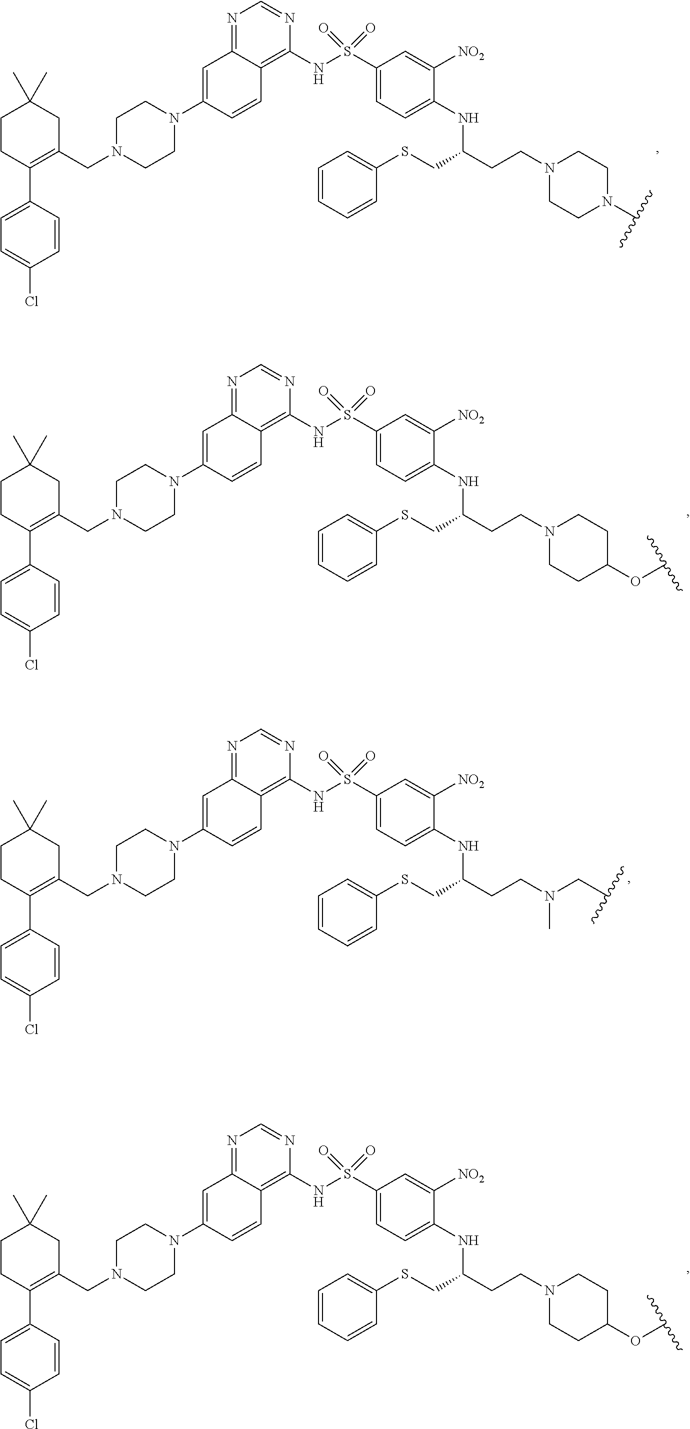 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

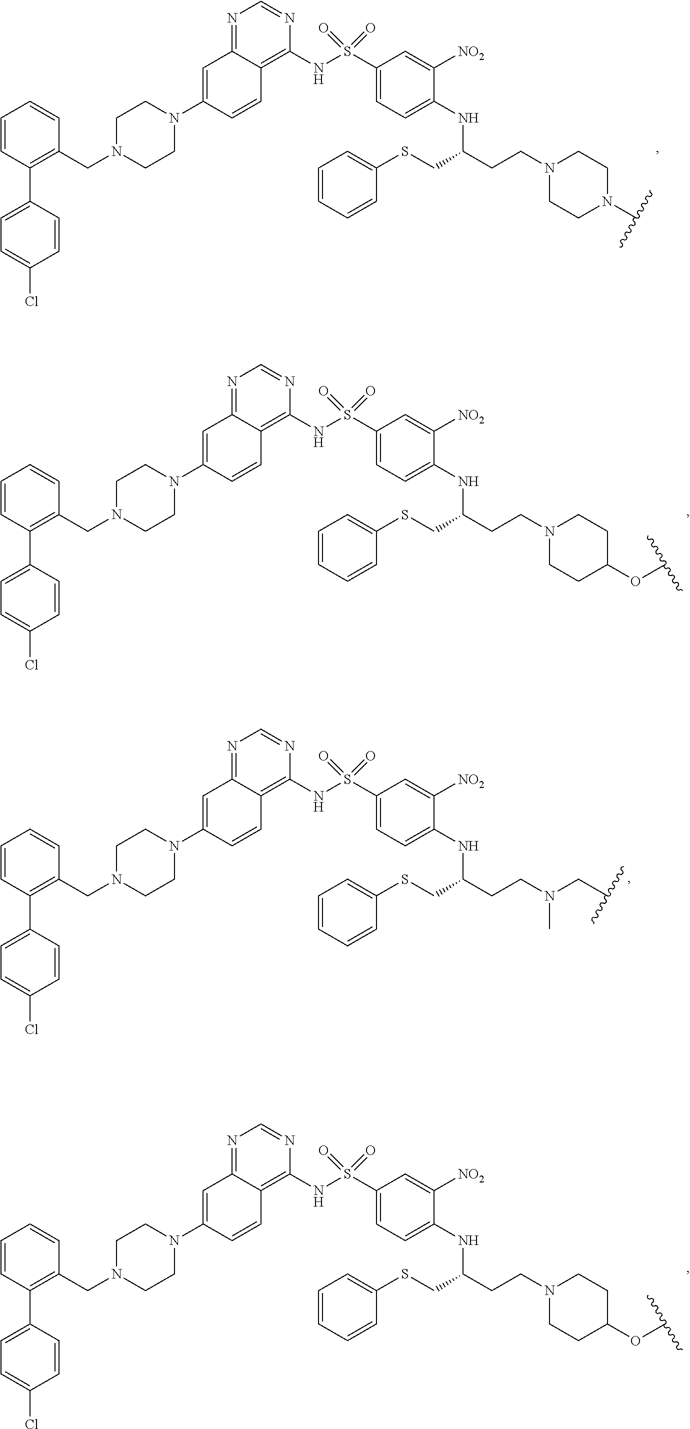 [SMILES: *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *CN(C)CC[C@H](CSc1ccccc1)Nc1ccc(S(=O)(=O)Nc2ncnc3cc(N4CCN(Cc5ccccc5-c5ccc(Cl)cc5)CC4)ccc23)cc1[N+](=O)[O-] ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

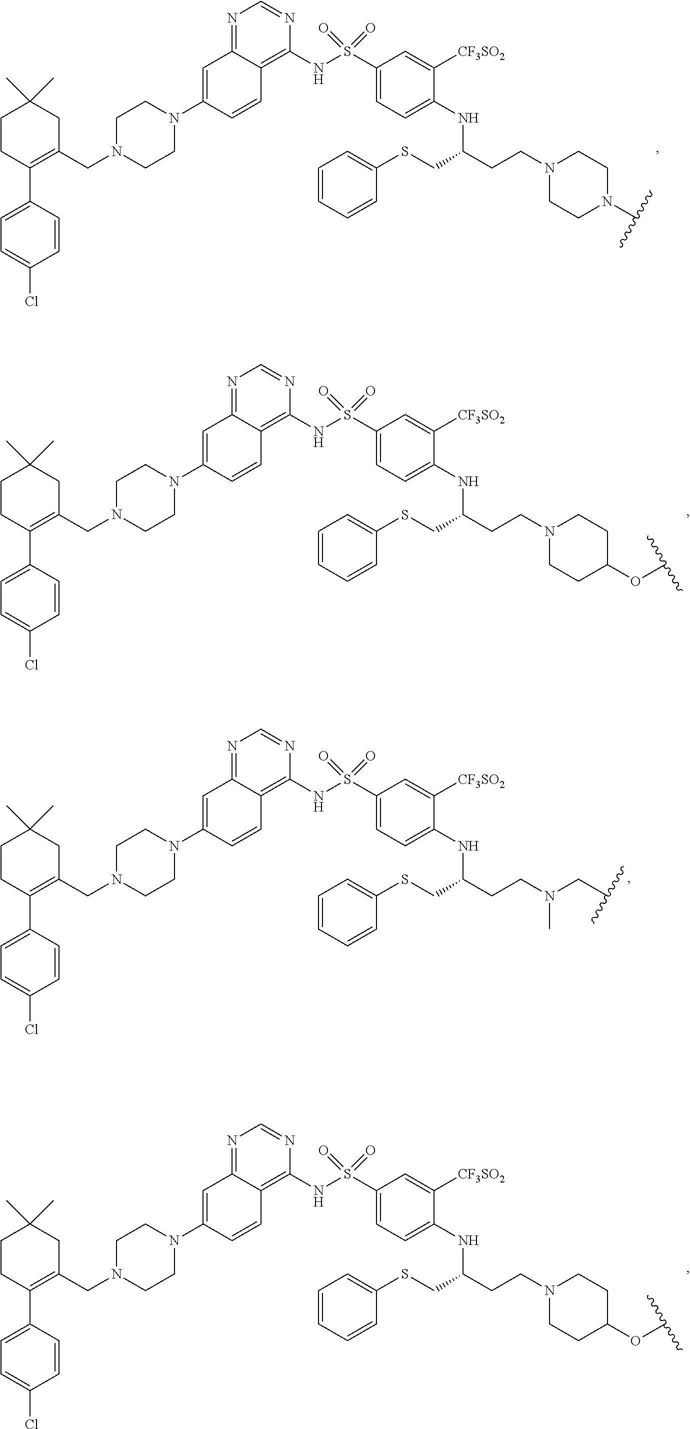

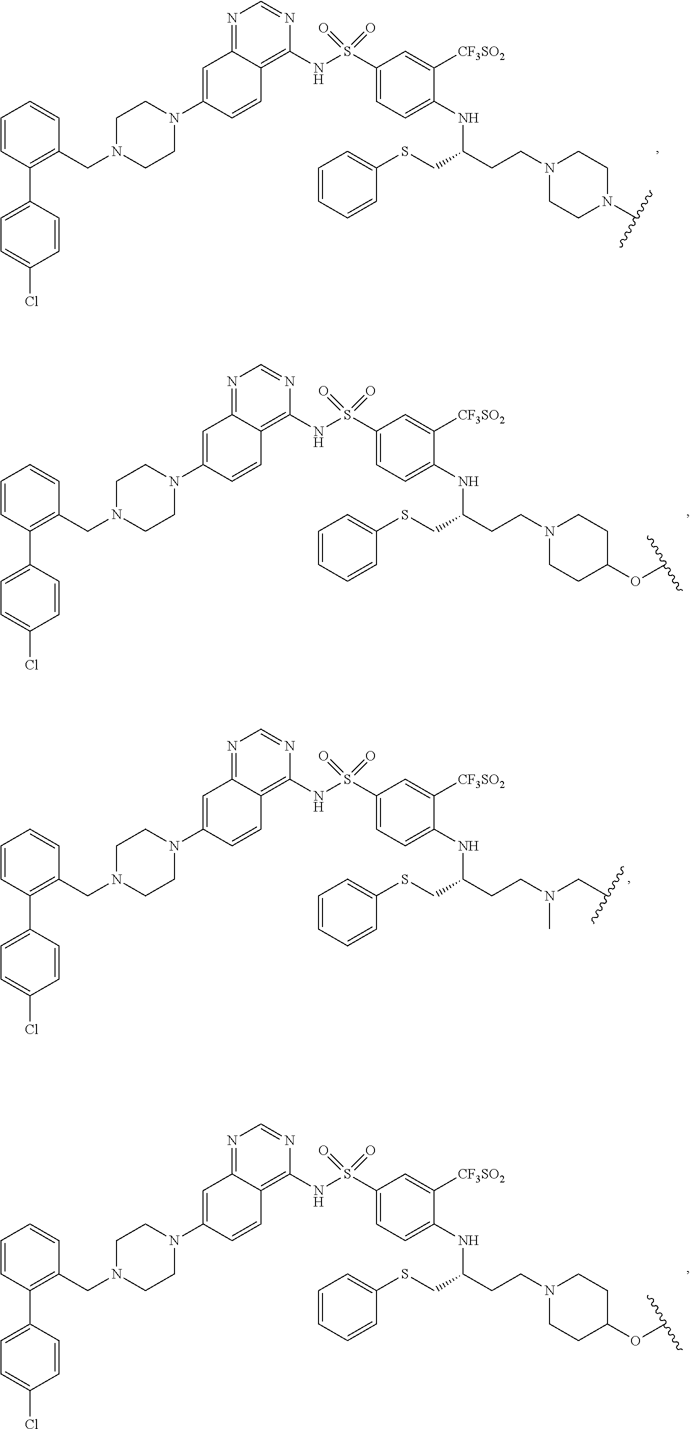

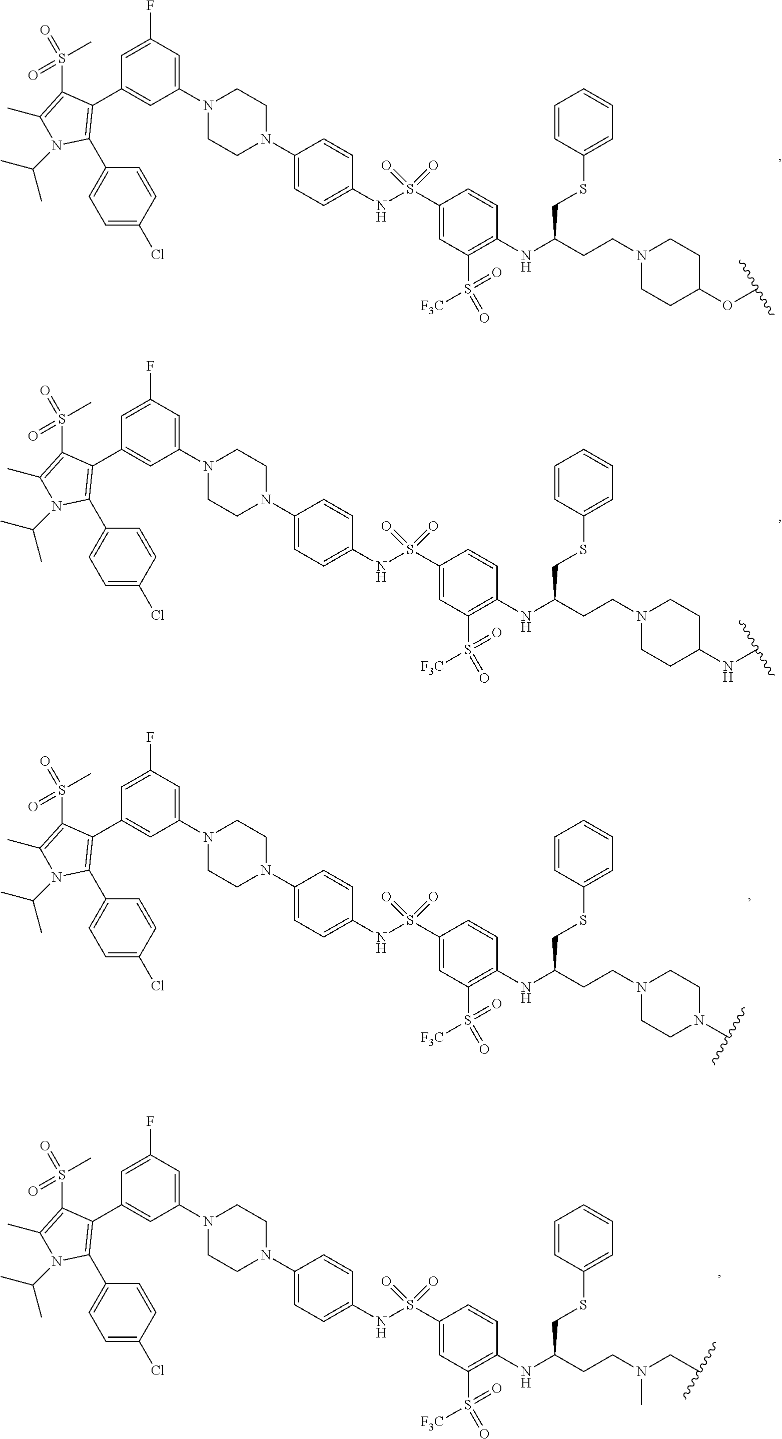

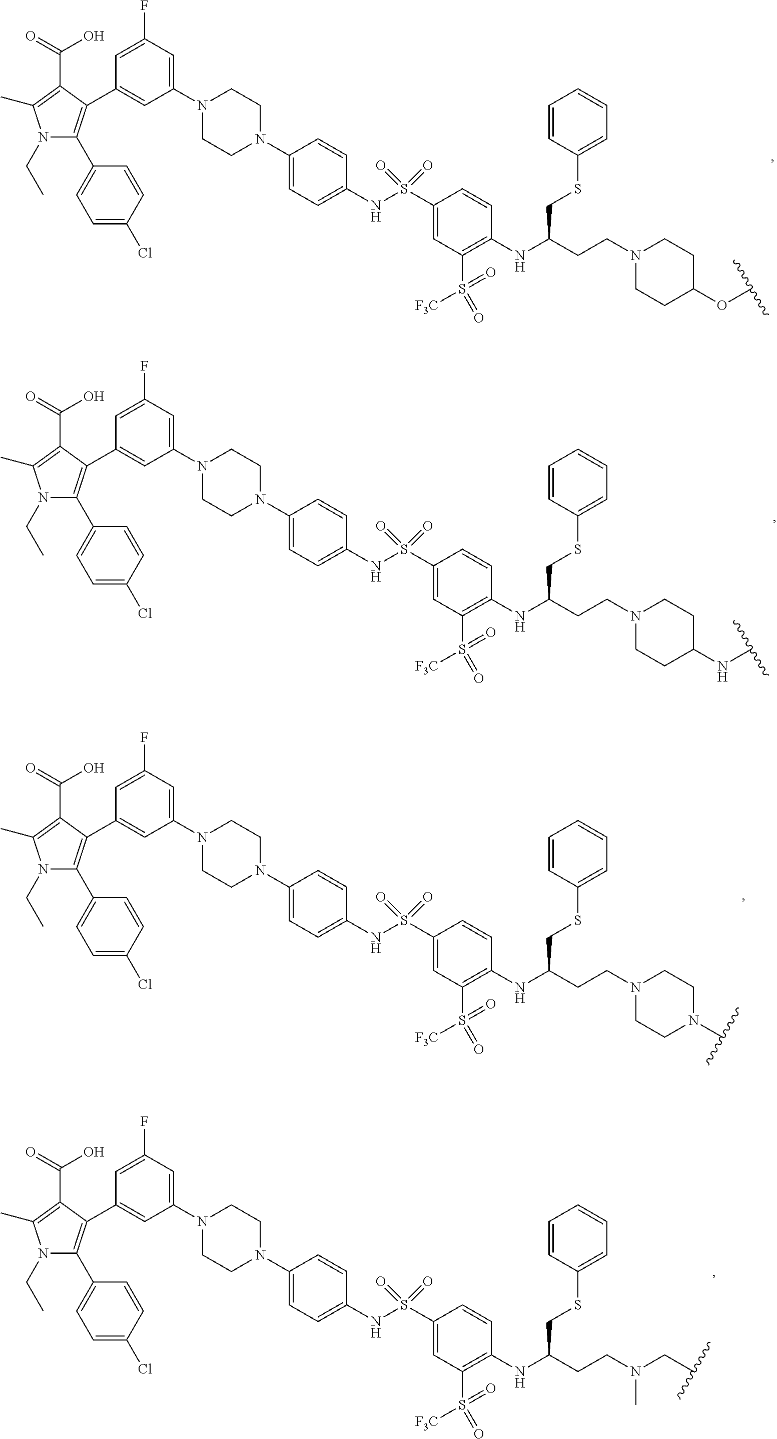

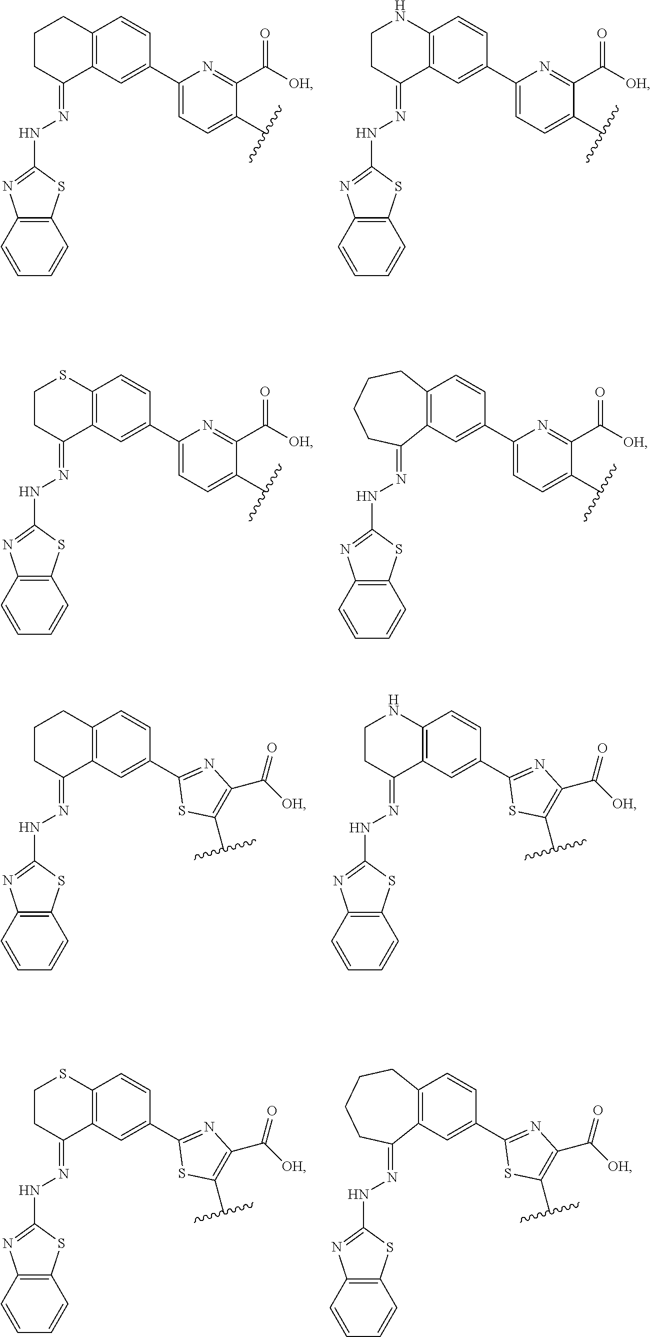

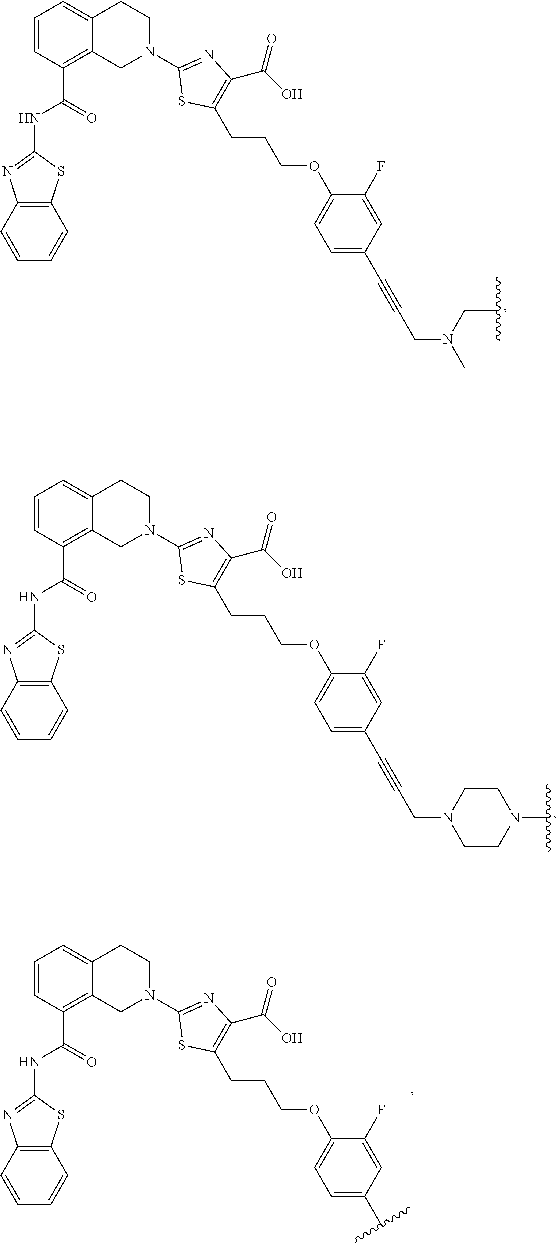 [SMILES: *CN(C)CC#Cc1ccc(OCCCc2sc(N3CCc4cccc(C(=O)Nc5nc6ccccc6s5)c4C3)nc2C(=O)O)c(F)c1 ; *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1]

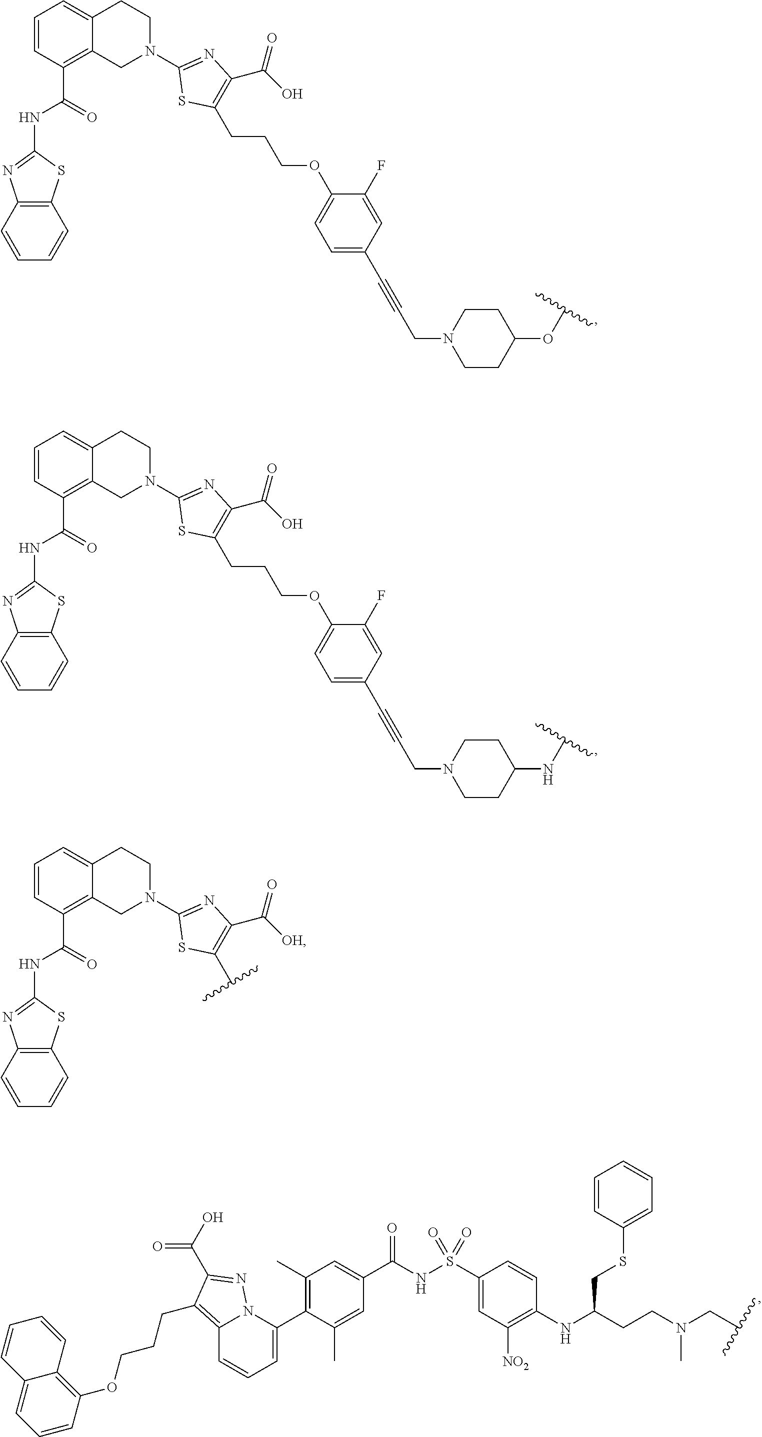 [SMILES: *OC1CCN(CC#Cc2ccc(OCCCc3sc(N4CCc5cccc(C(=O)Nc6nc7ccccc7s6)c5C4)nc3C(=O)O)c(F)c2)CC1 ; *NC1CCN(CC#Cc2ccc(OCCCc3sc(N4CCc5cccc(C(=O)Nc6nc7ccccc7s6)c5C4)nc3C(=O)O)c(F)c2)CC1 ; *c1sc(N2CCc3cccc(C(=O)Nc4nc5ccccc5s4)c3C2)nc1C(=O)O ; *CN(C)CC[C@H](CSc1ccccc1)Nc1ccc(S(=O)(=O)NC(=O)c2cc(C)c(-c3cccc4c(CCCOc5cccc6ccccc56)c(C(=O)O)nn34)c(C)c2)cc1[N+](=O)[O-]]

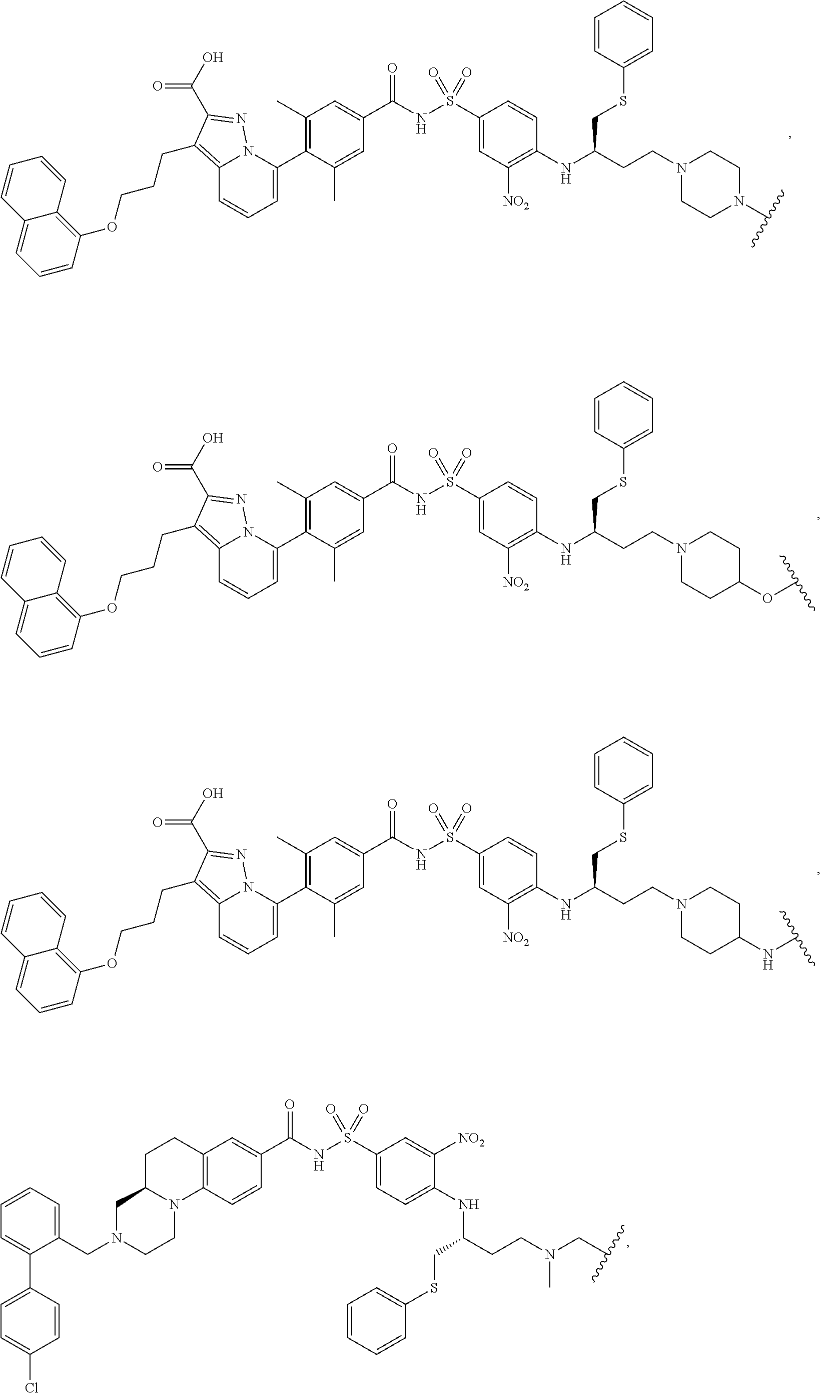

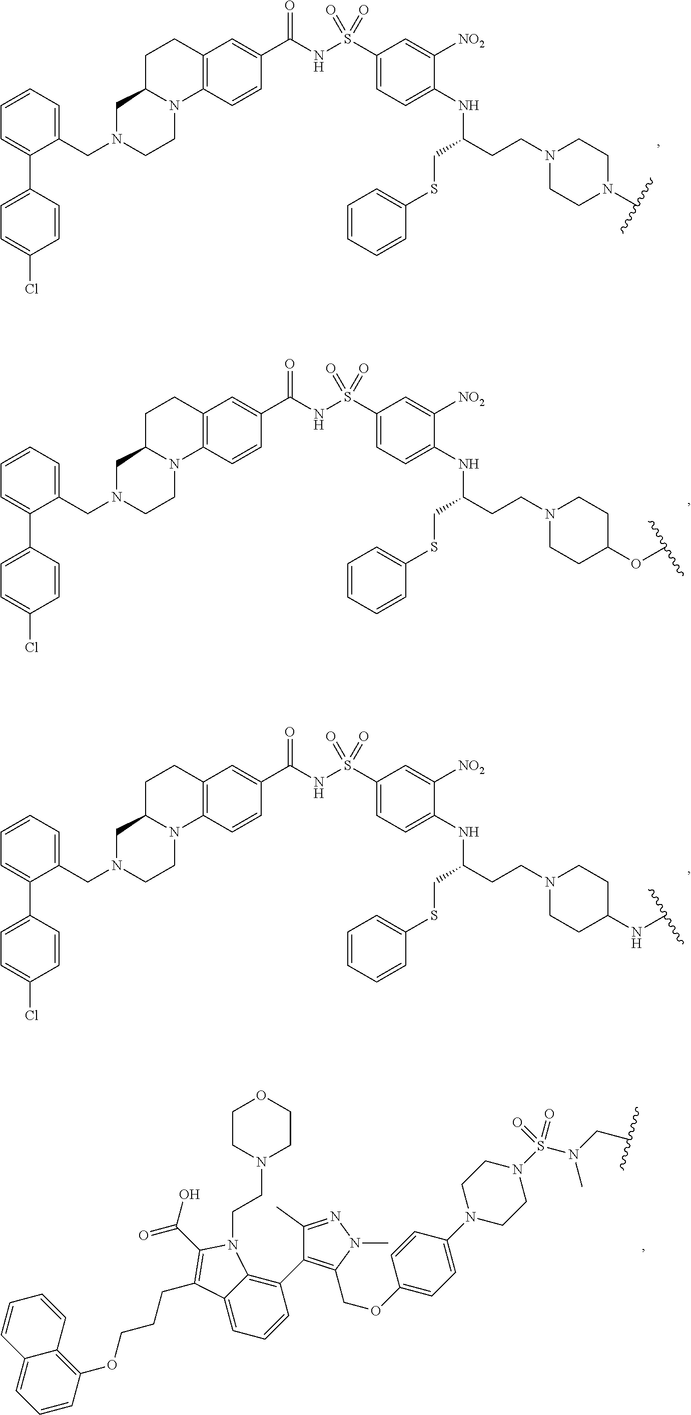 [SMILES: *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1]

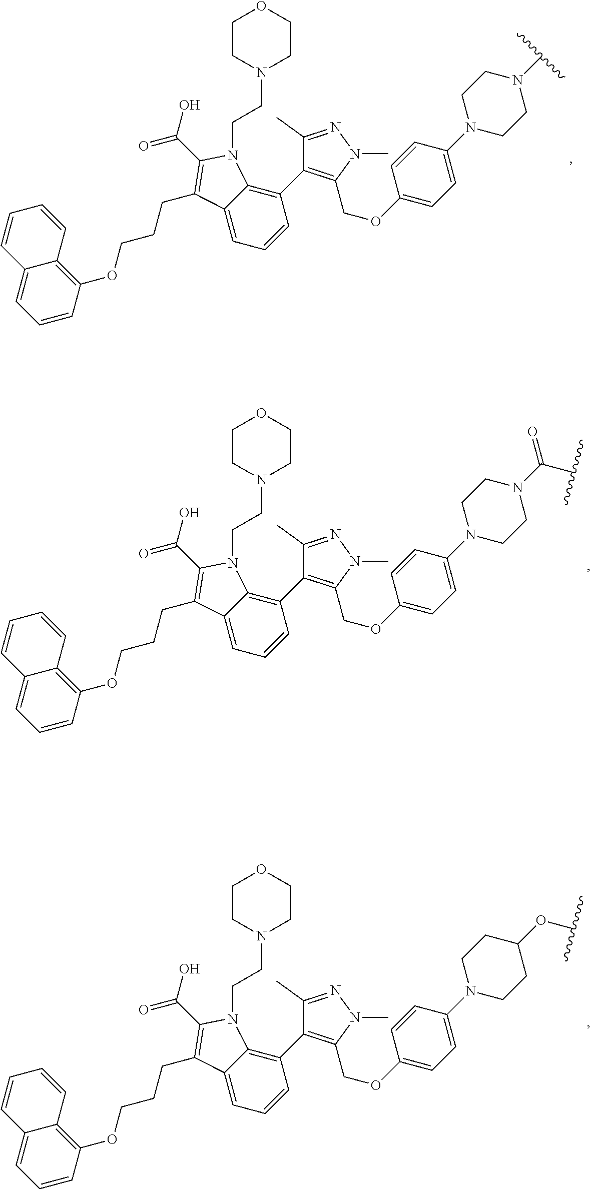 [SMILES: *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *OC1CCN(c2ccc(OCc3c(-c4cccc5c(CCCOc6cccc7ccccc67)c(C(=O)O)n(CCN6CCOCC6)c45)c(C)nn3C)cc2)CC1]

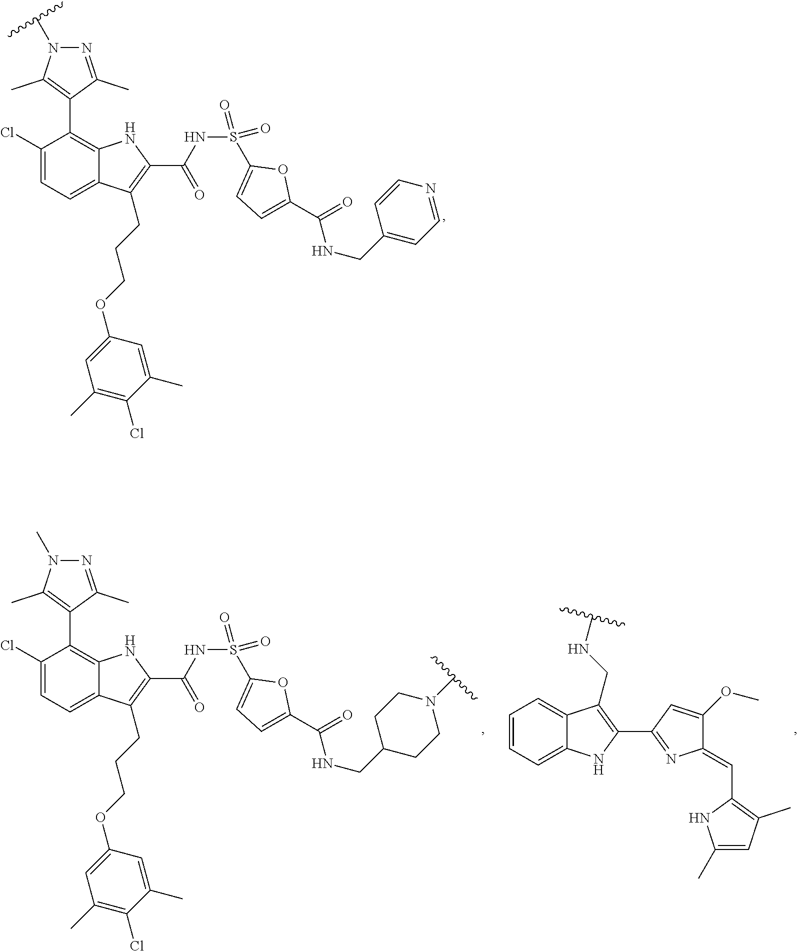 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12]

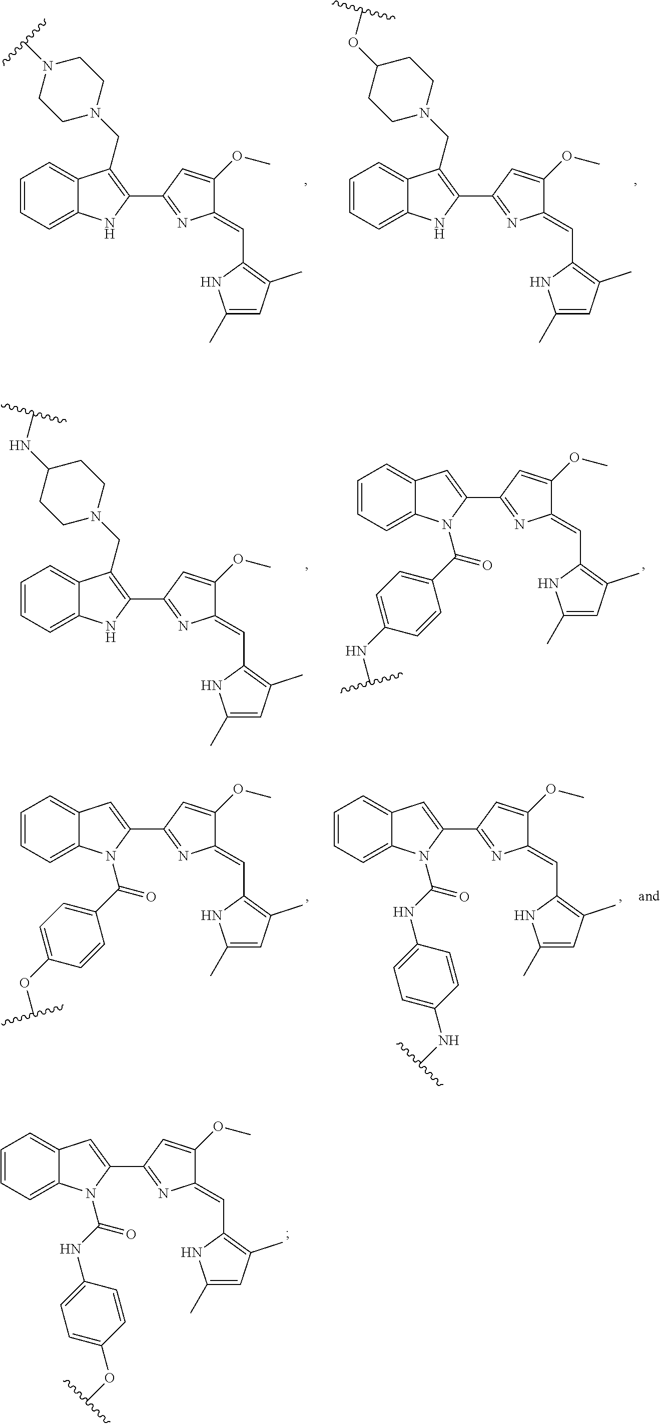

R³ is absent, a bond, or a substituted or unsubstituted C₁-C₁₀ alkyl;

A is absent, a bond, a substituted or unsubstituted C₁-C₆ aryl, a substituted or unsubstituted C₁-C₆ cycloalkyl, a substituted or unsubstituted C₁-C₆ heterocyclic group;

R⁴ is a bond or a substituted or unsubstituted C₁-C₁₀ alky;

n is an integer from 0 to 5;

R² is selected from the group consisting of

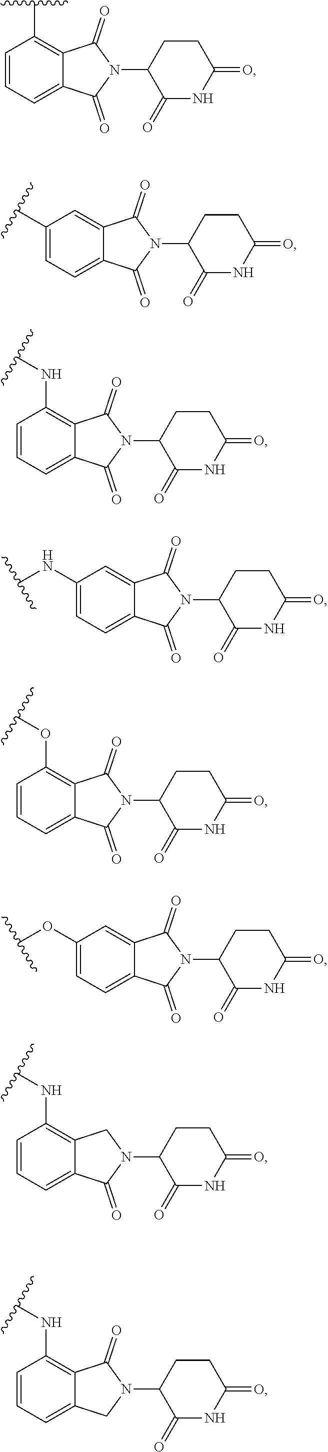

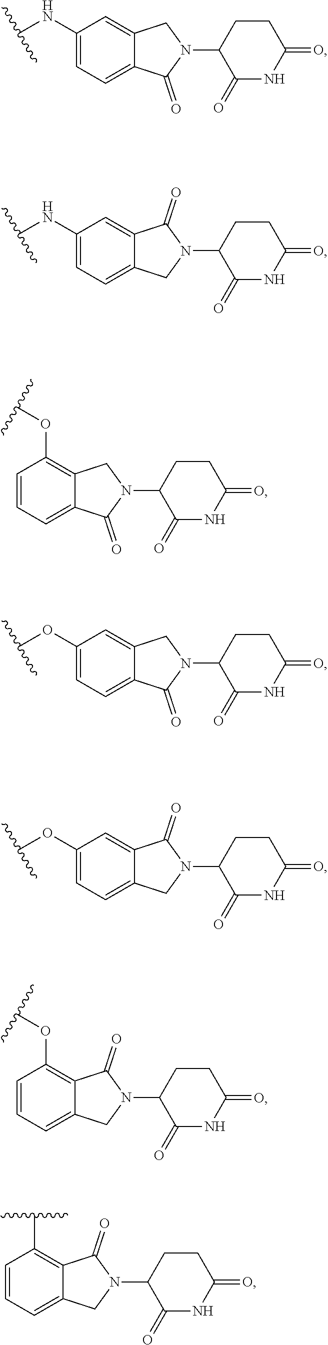

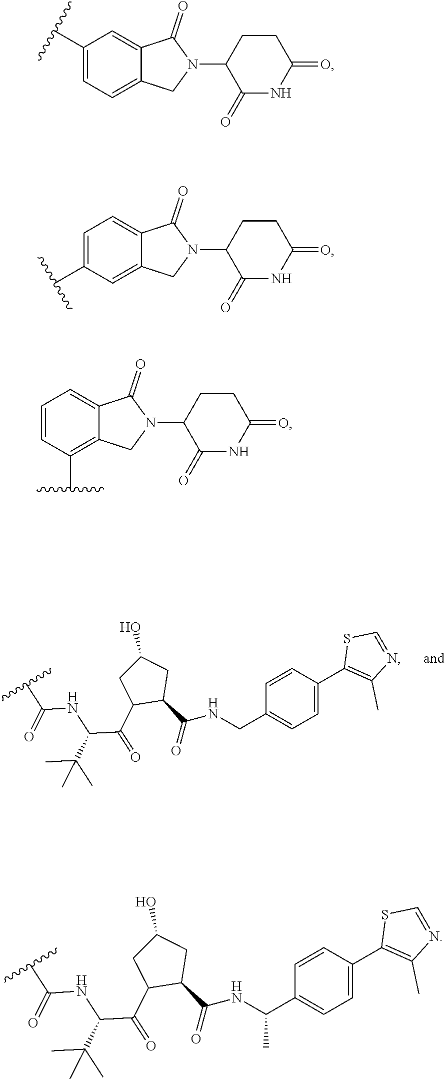 [SMILES: *c1ccc2c(c1)C(=O)N(C1CCC(=O)NC1=O)C2 ; *c1ccc2c(c1)CN(C1CCC(=O)NC1=O)C2=O ; *c1cccc2c1CN(C1CCC(=O)NC1=O)C2=O ; *C(=O)N[C@H](C(=O)C1C[C@H](O)C[C@H]1C(=O)NCc1ccc(-c2scnc2C)cc1)C(C)(C)C ; *C(=O)N[C@H](C(=O)C1C[C@H](O)C[C@H]1C(=O)N[C@@H](C)c1ccc(-c2scnc2C)cc1)C(C)(C)C]

In an embodiment, a compound of Formula (II) comprises any of the preceding compounds of Formula (II), wherein R¹ may be

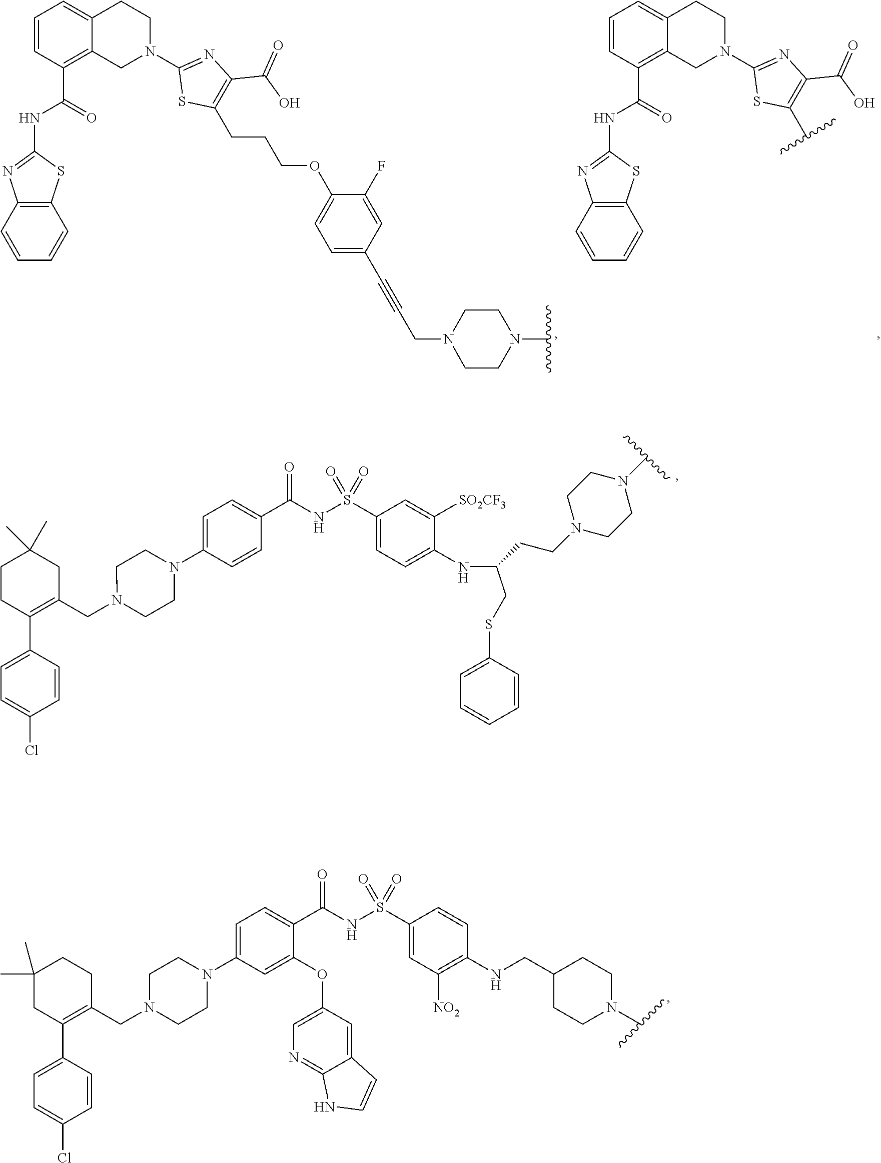

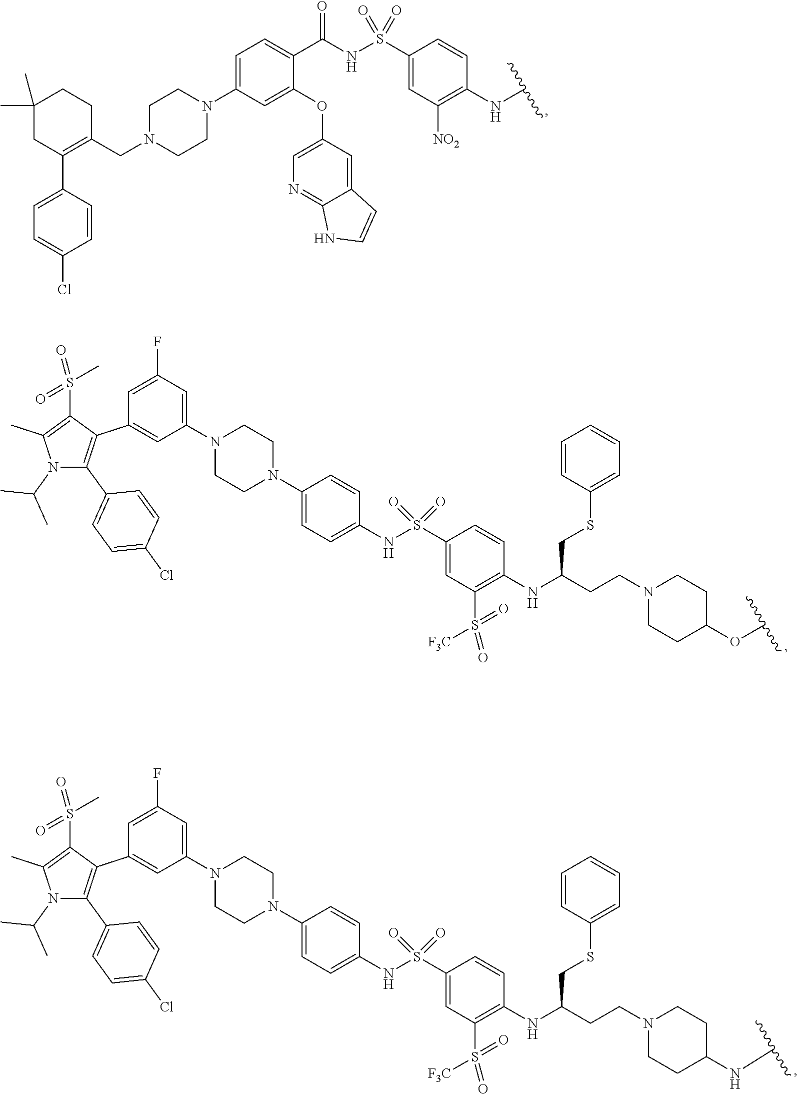 [SMILES: *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-] ; *OC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

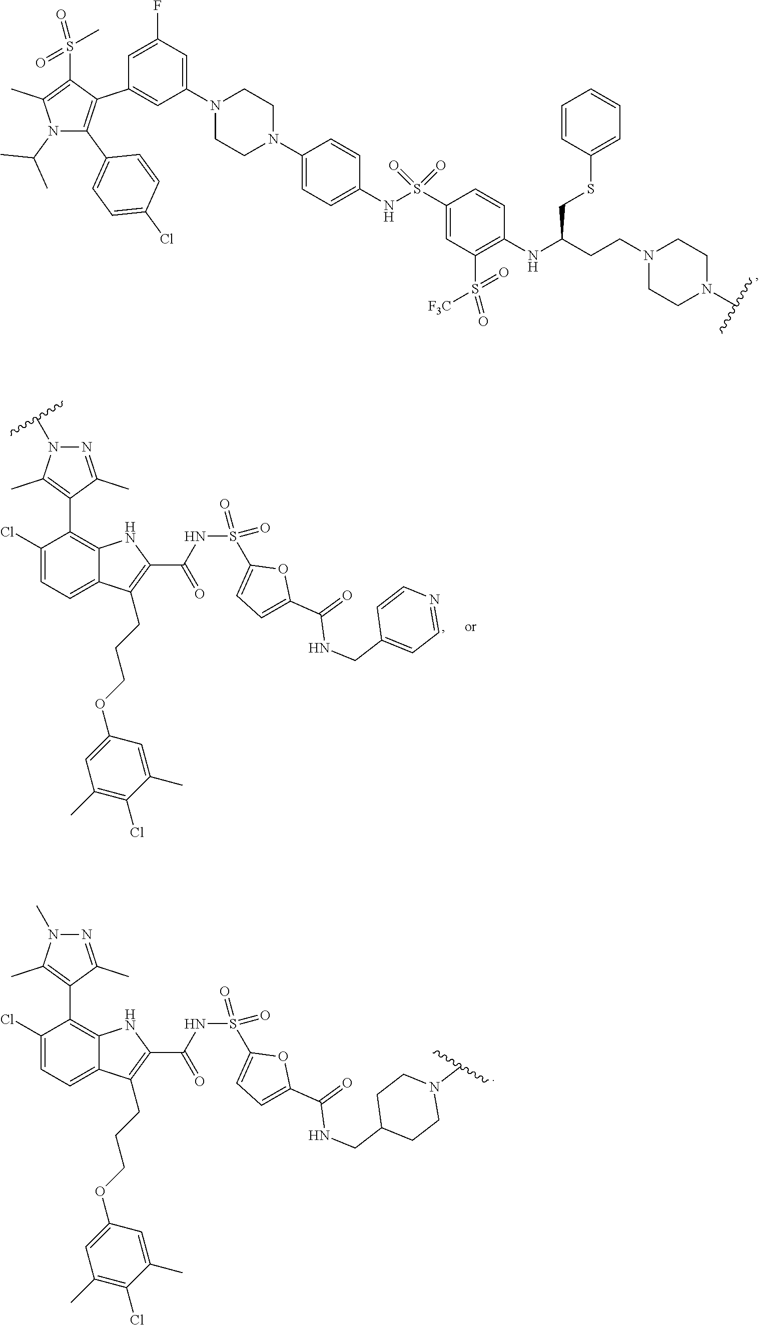 [SMILES: *N1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1]

In an embodiment, a compound of Formula (II) comprises any of the preceding compounds of Formula (II), wherein R³ may be absent, an unsubstituted C₁-C₆ alkyl, or a substituted or unsubstituted C₃-C₆ ketone.

In a preferred embodiment, a compound of Formula (II) comprises any of the preceding compounds of Formula (II), wherein R³ may be absent, a bond, an unsubstituted C₁-C₃ alkyl, or an unsubstituted C₃-C₆ ketone.

In still a preferred embodiment, a compound of Formula (II) comprises any of the preceding compounds of Formula (II), wherein R³ may be absent, a bond, 2-pentanone, or an unsubstituted C₂-C₃ alkyl.

In another embodiment, a compound of Formula (II) comprises any of the preceding compounds of Formula (II), wherein A may be absent, a bond, or a substituted or unsubstituted C₁-C₆ heterocyclic group.

In a preferred embodiment, a compound of Formula (II) comprises any of the preceding compounds of Formula (II), wherein A may be absent, a bond, or an unsubstituted C₅ heterocyclic group.

In still a preferred embodiment, a compound of Formula (II) comprises any of the preceding compounds of Formula (II), wherein A may be absent, a bond, or a triazole.

In another embodiment, a compound of Formula (II) comprises any of the preceding compounds of Formula (II), wherein n may be 0 to 3.

In a preferred embodiment, a compound of Formula (II) comprises any of the preceding compounds of Formula (II), wherein n may be 0 to 2.

In still a preferred embodiment, a compound of Formula (II) comprises any of the preceding compounds of Formula (II), wherein n may be 1 to 2.

In another embodiment, a compound of Formula (II) comprises any of the preceding compounds of Formula (II), wherein R⁴ may be a bond or a substituted or unsubstituted C₁-C₁₀ alkyl.

In a preferred embodiment, a compound of Formula (II) comprises any of the preceding compounds of Formula (II), wherein R⁴ may be a bond or a substituted C₁-C₁₀ alkyl.

In another embodiment, a compound of Formula (II) comprises any of the preceding compounds of Formula (II), wherein R² is

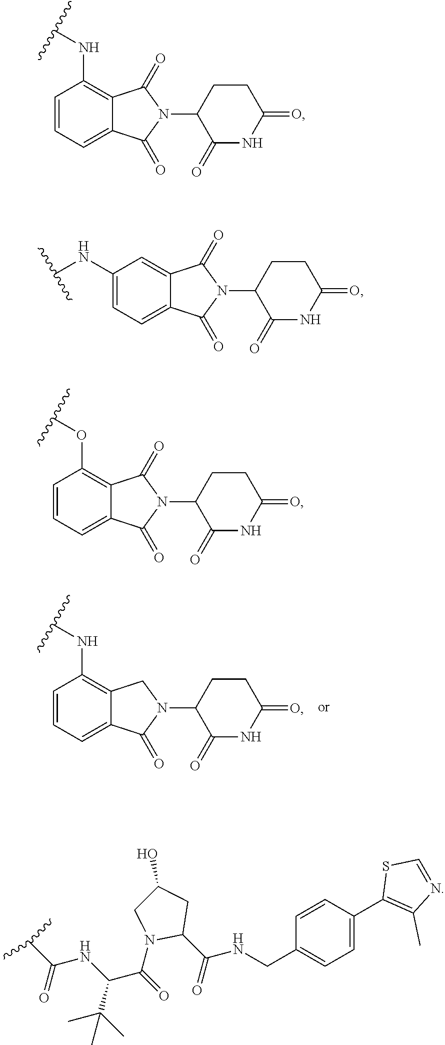 [SMILES: *Nc1cccc2c1C(=O)N(C1CCC(=O)NC1=O)C2=O ; *Nc1ccc2c(c1)C(=O)N(C1CCC(=O)NC1=O)C2=O ; *Oc1cccc2c1C(=O)N(C1CCC(=O)NC1=O)C2=O ; *Nc1cccc2c1CN(C1CCC(=O)NC1=O)C2=O ; "or" ; *C(=O)N[C@H](C(=O)N1C[C@H](O)CC1C(=O)NCc1ccc(-c2scnc2C)cc1)C(C)(C)C]

In one embodiment, a compound of the disclosure comprises Formula (II), wherein R¹ may be

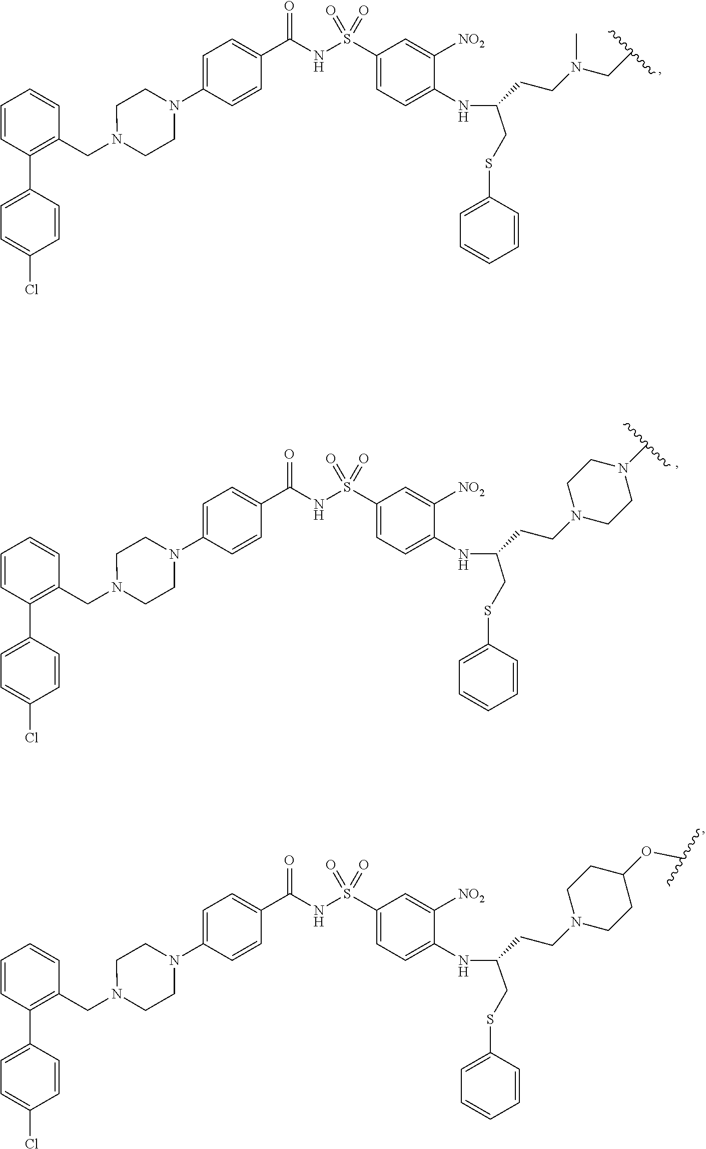 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

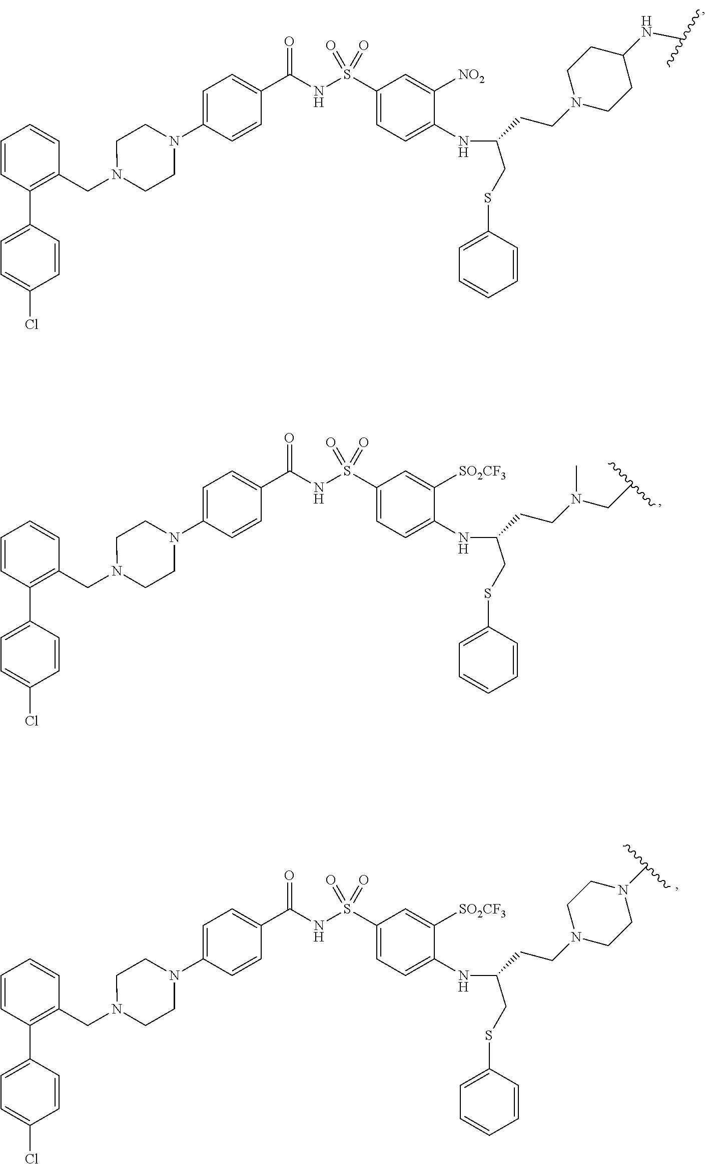

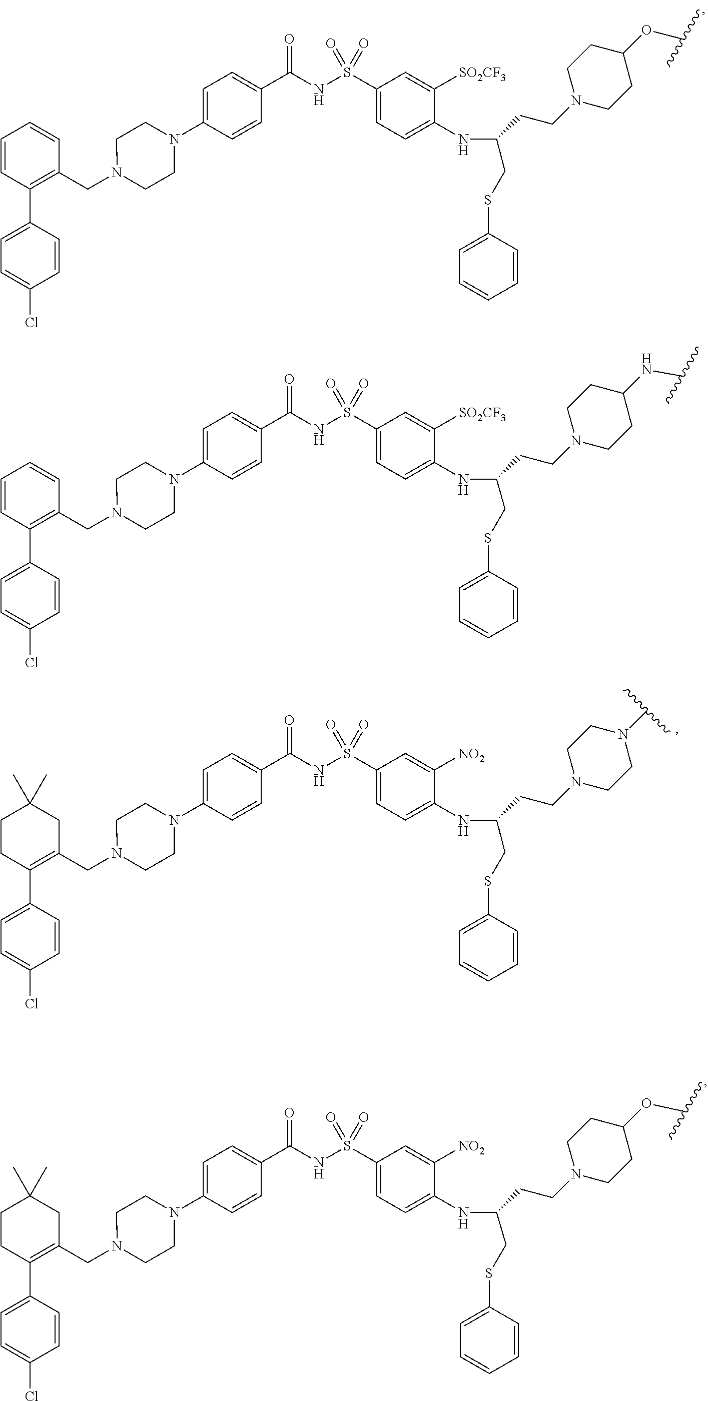

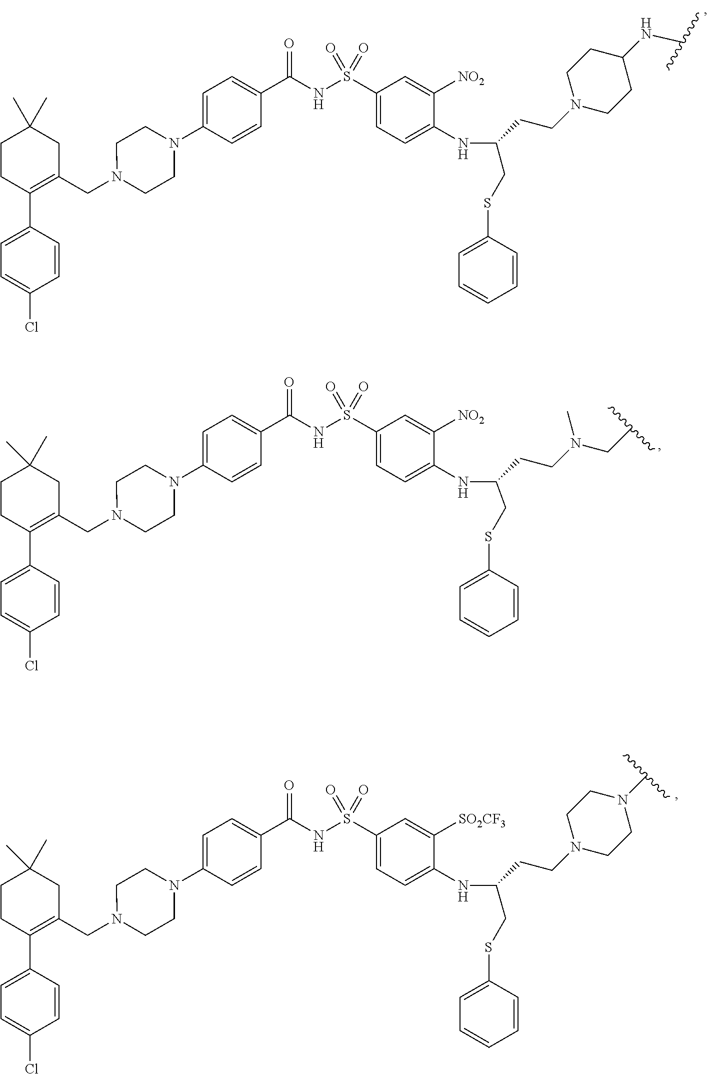

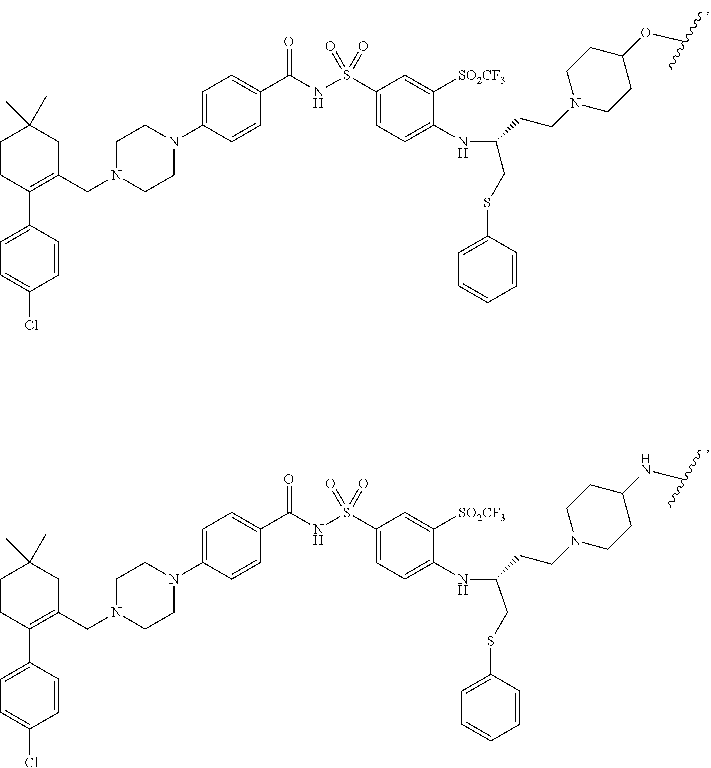

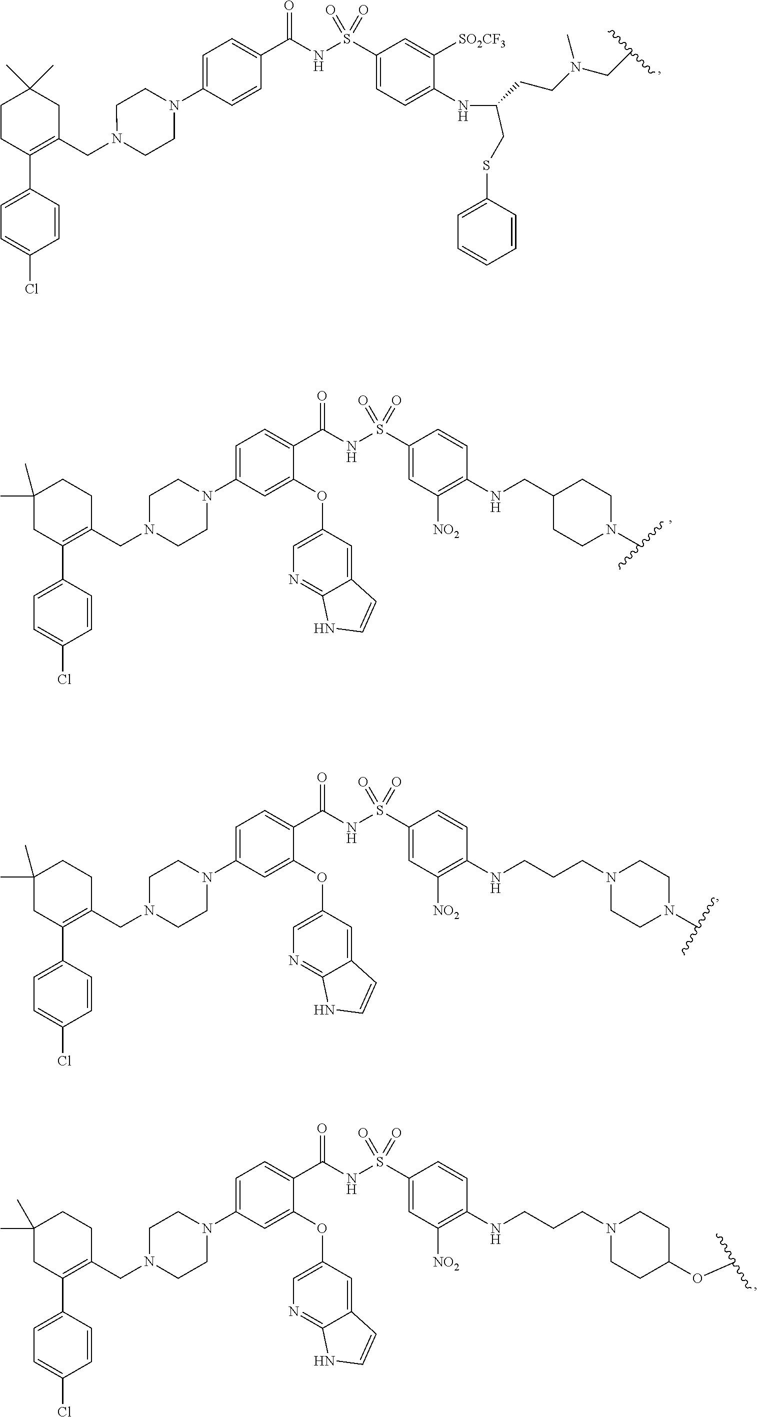

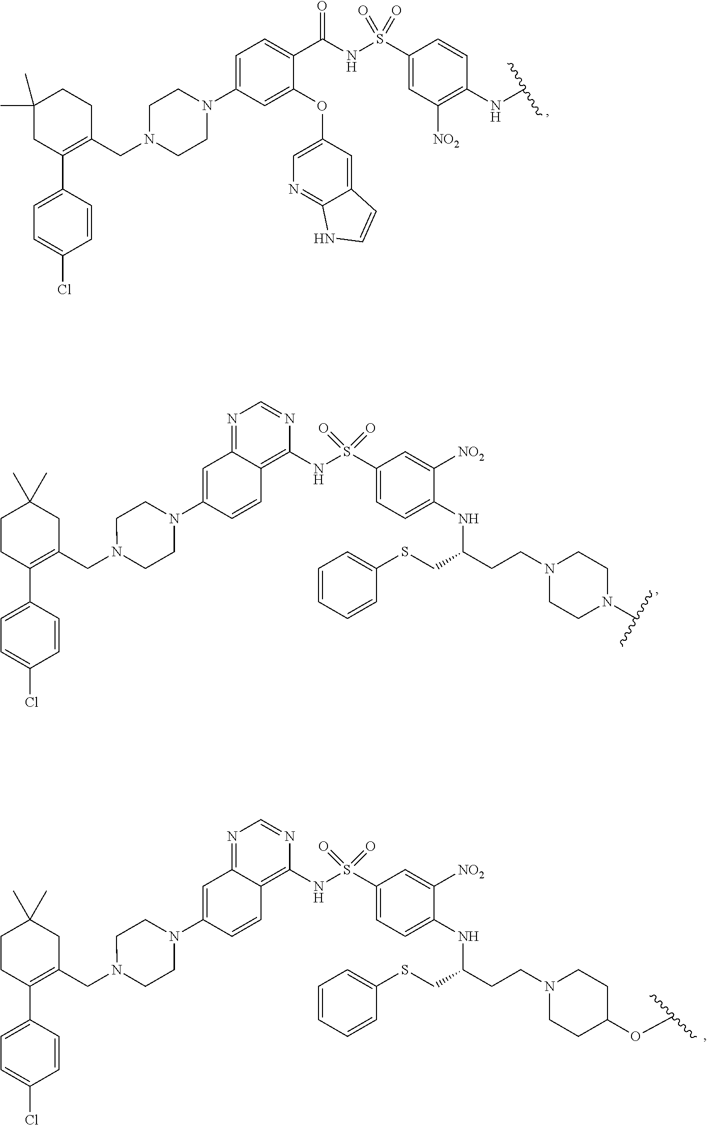 [SMILES: *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-] ; *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

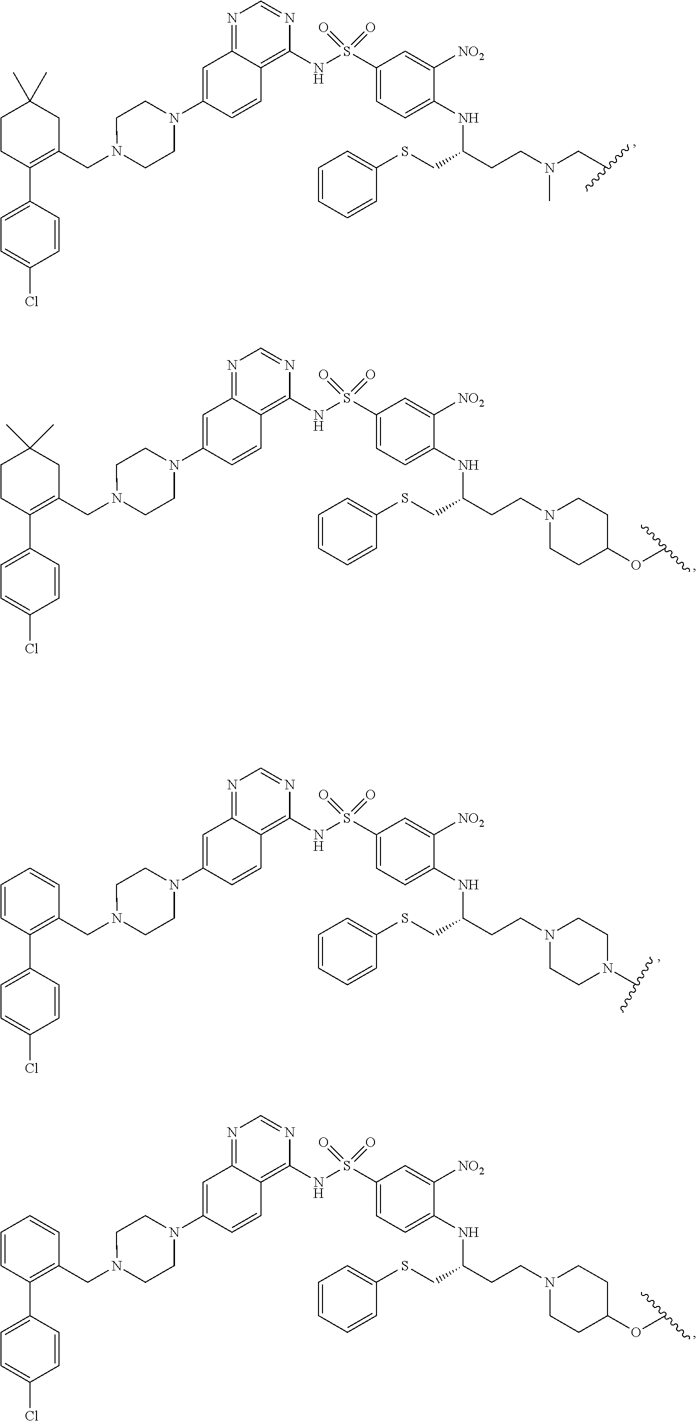 [SMILES: *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

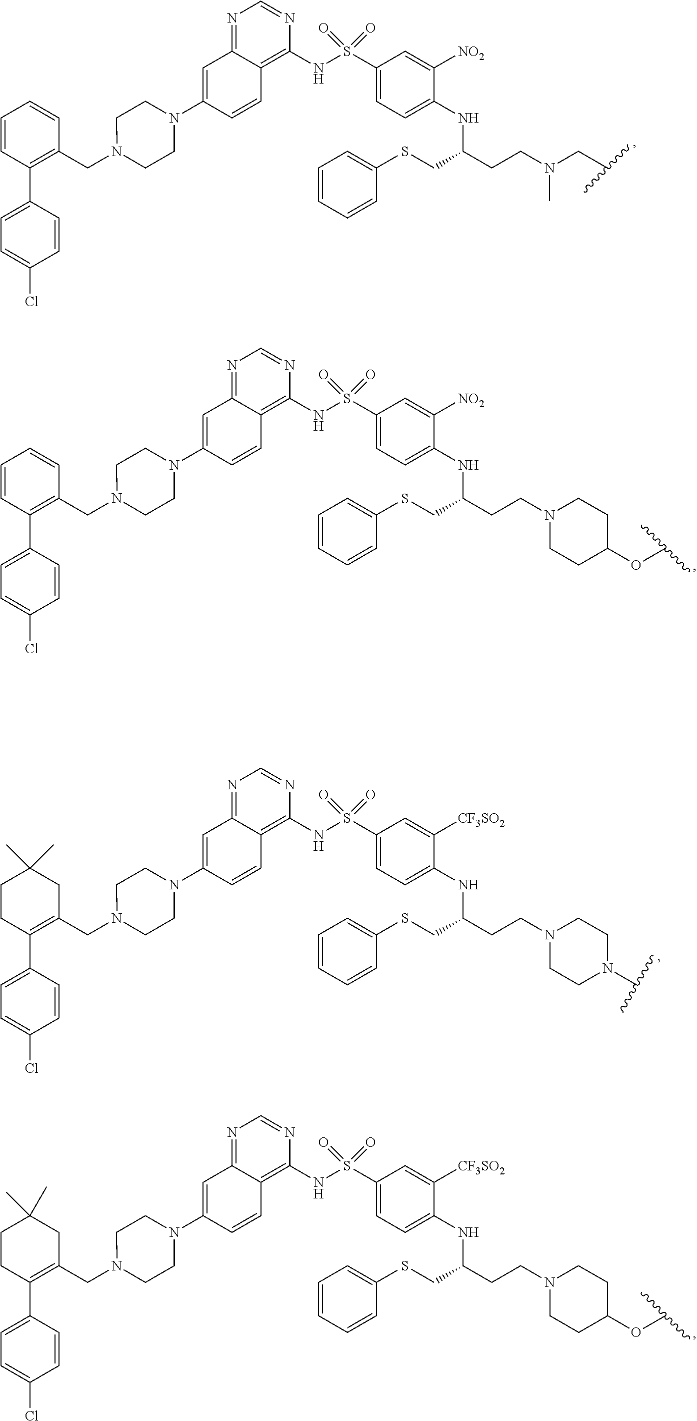

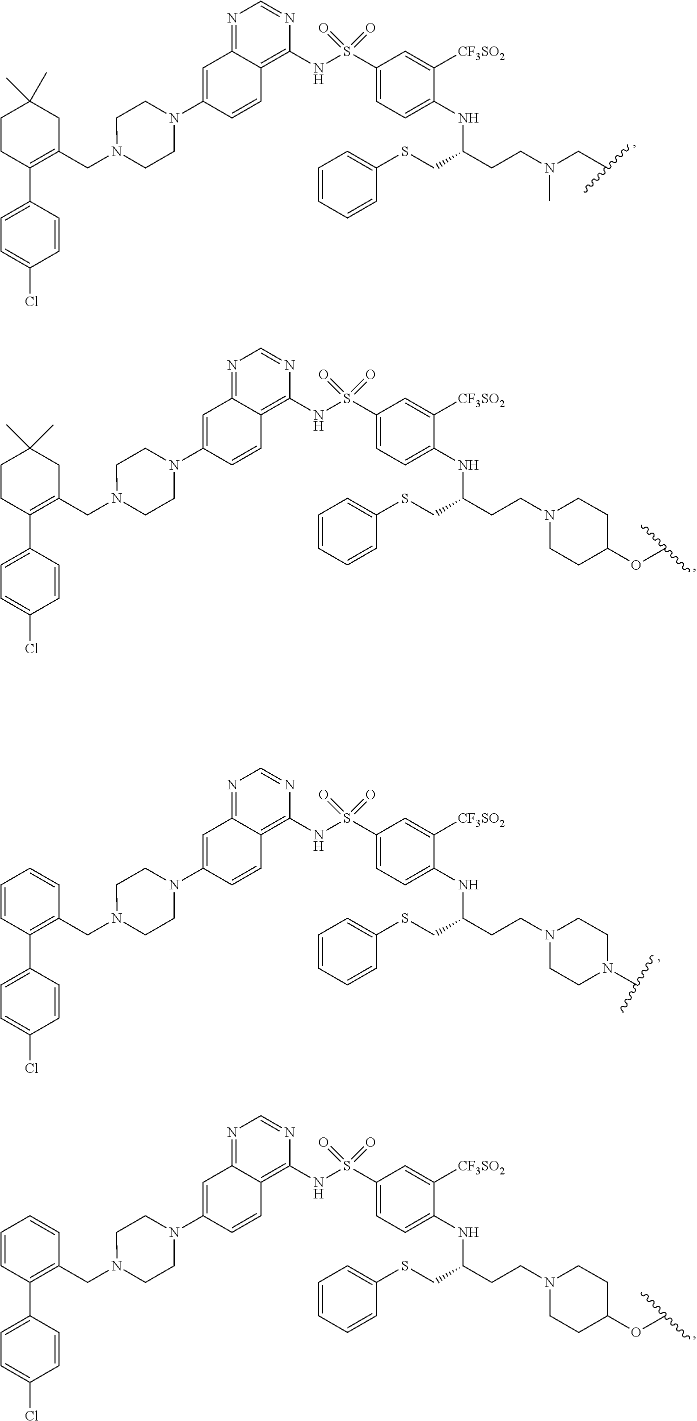

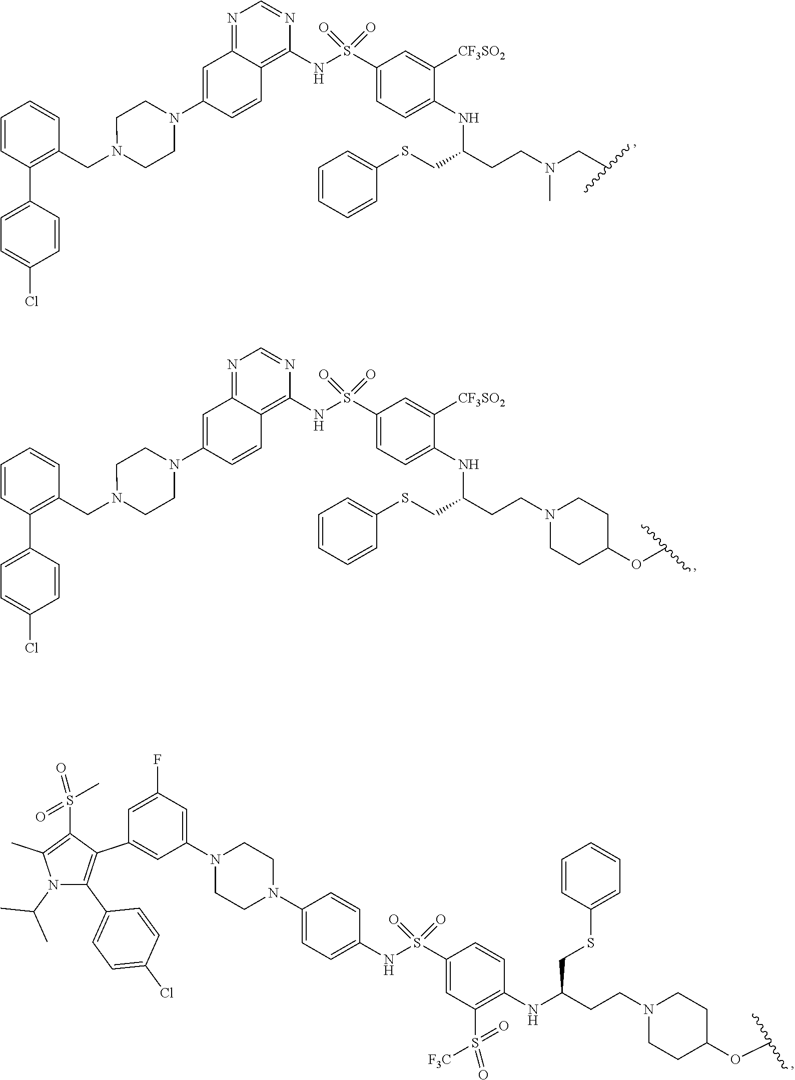

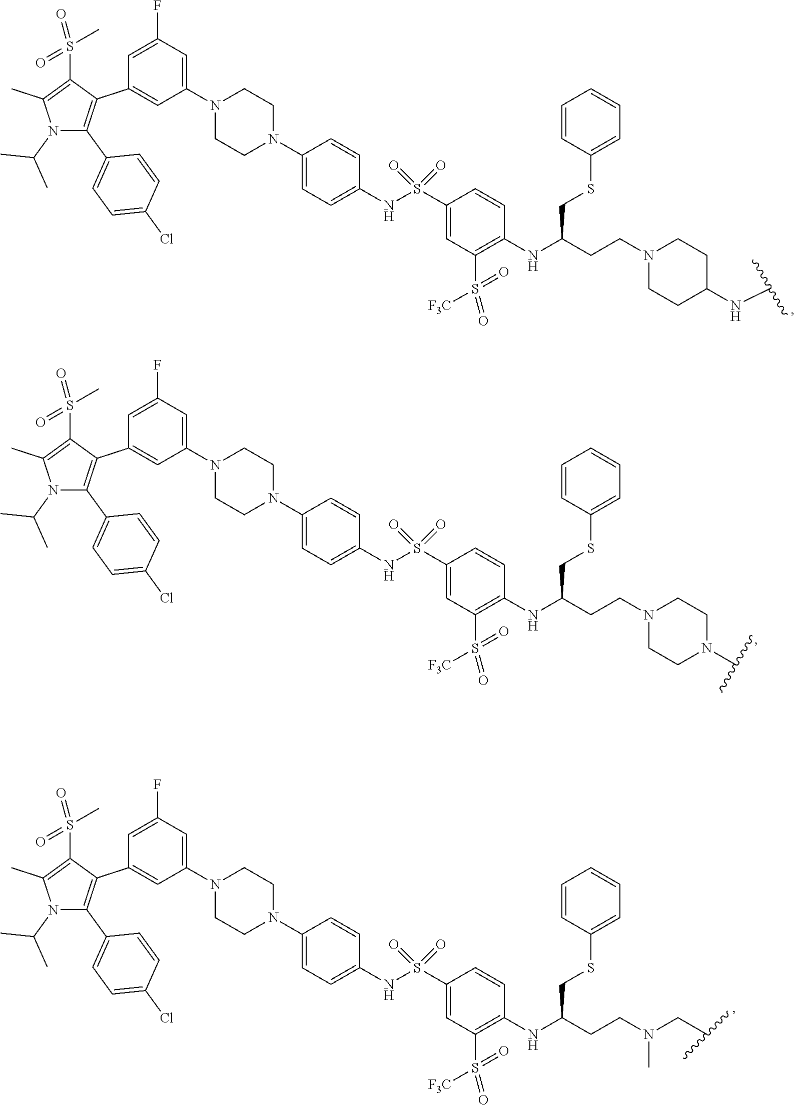 [SMILES: *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *N1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *CN(C)CC[C@H](CSc1ccccc1)Nc1ccc(S(=O)(=O)Nc2ccc(N3CCN(c4cc(F)cc(-c5c(S(C)(=O)=O)c(C)n(C(C)C)c5-c5ccc(Cl)cc5)c4)CC3)cc2)cc1S(=O)(=O)C(F)(F)F]

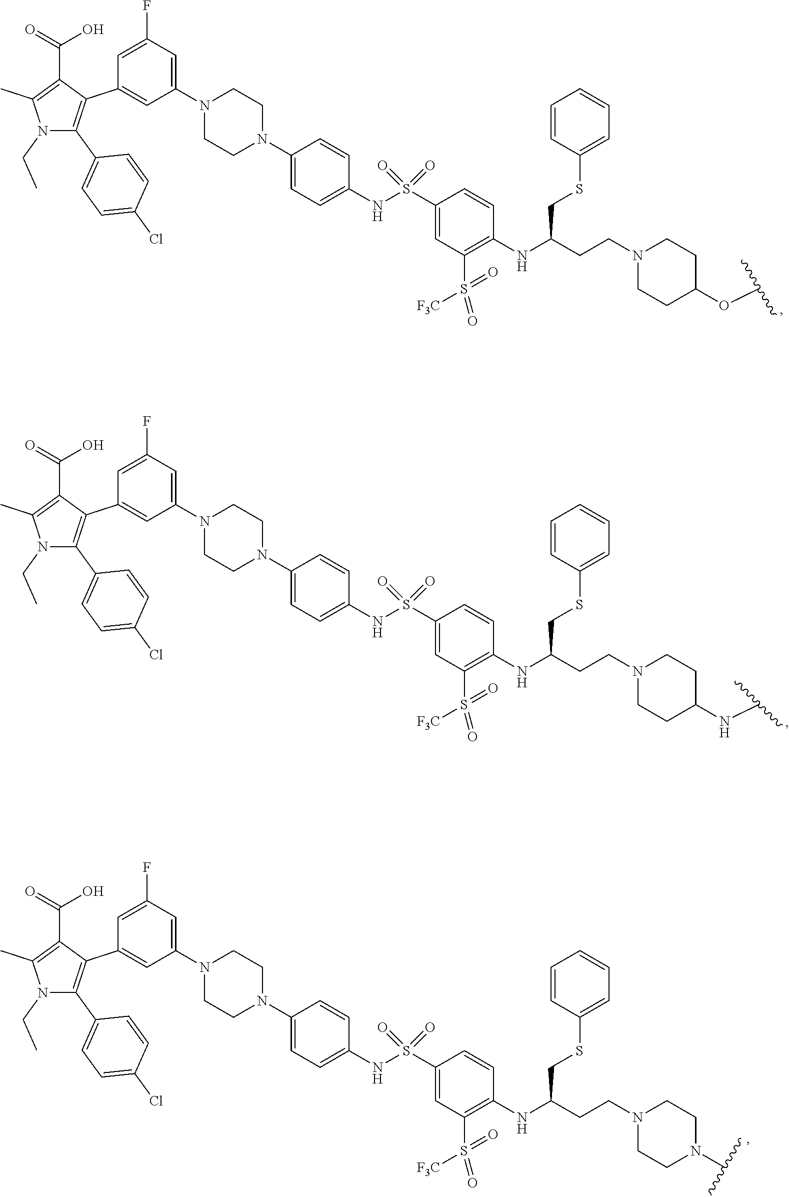 [SMILES: *OC1CCN(CC[C@H](CSc2ccccc2)Nc2ccc(S(=O)(=O)Nc3ccc(N4CCN(c5cc(F)cc(-c6c(C(=O)O)c(C)n(CC)c6-c6ccc(Cl)cc6)c5)CC4)cc3)cc2S(=O)(=O)C(F)(F)F)CC1 ; *NC1CCN(CC[C@H](CSc2ccccc2)Nc2ccc(S(=O)(=O)Nc3ccc(N4CCN(c5cc(F)cc(-c6c(C(=O)O)c(C)n(CC)c6-c6ccc(Cl)cc6)c5)CC4)cc3)cc2S(=O)(=O)C(F)(F)F)CC1 ; *N1CCN(CC[C@H](CSc2ccccc2)Nc2ccc(S(=O)(=O)Nc3ccc(N4CCN(c5cc(F)cc(-c6c(C(=O)O)c(C)n(CC)c6-c6ccc(Cl)cc6)c5)CC4)cc3)cc2S(=O)(=O)C(F)(F)F)CC1]

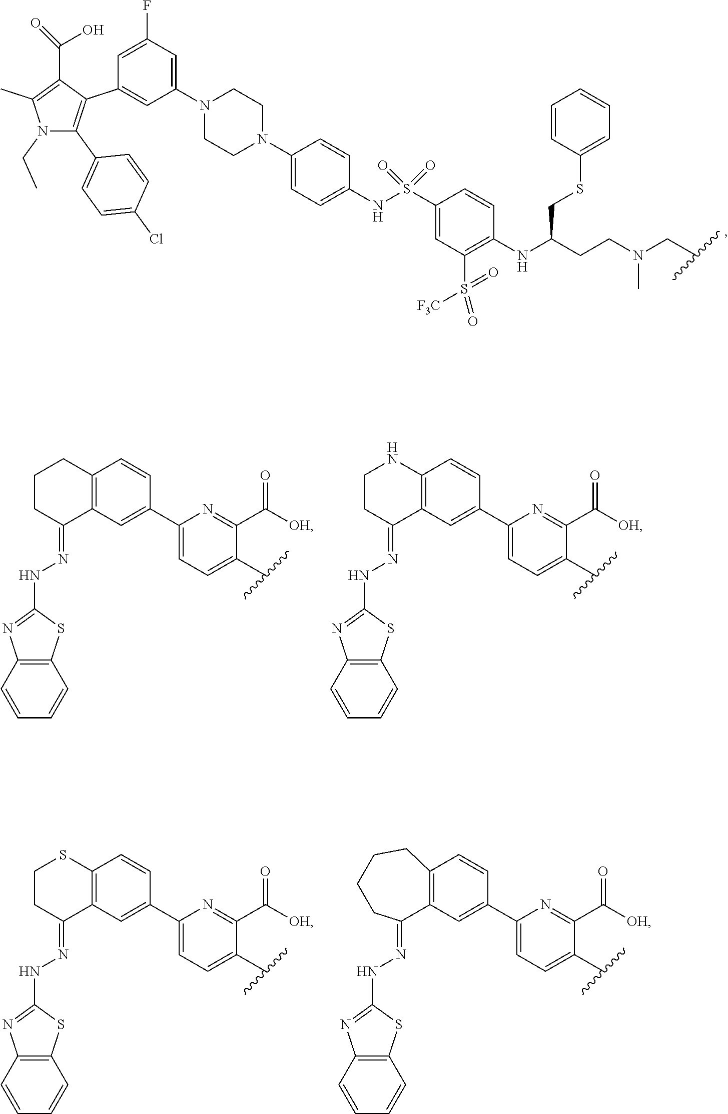 [SMILES: *CN(C)CC[C@H](CSc1ccccc1)Nc1ccc(S(=O)(=O)Nc2ccc(N3CCN(c4cc(F)cc(-c5c(C(=O)O)c(C)n(CC)c5-c5ccc(Cl)cc5)c4)CC3)cc2)cc1S(=O)(=O)C(F)(F)F ; *c1ccc(-c2ccc3c(c2)/C(=N/Nc2nc4ccccc4s2)CCC3)nc1C(=O)O ; *c1ccc(-c2ccc3c(c2)/C(=N/Nc2nc4ccccc4s2)CCN3)nc1C(=O)O ; *c1ccc(-c2ccc3c(c2)/C(=N/Nc2nc4ccccc4s2)CCS3)nc1C(=O)O ; *c1ccc(-c2ccc3c(c2)/C(=N/Nc2nc4ccccc4s2)CCCC3)nc1C(=O)O]

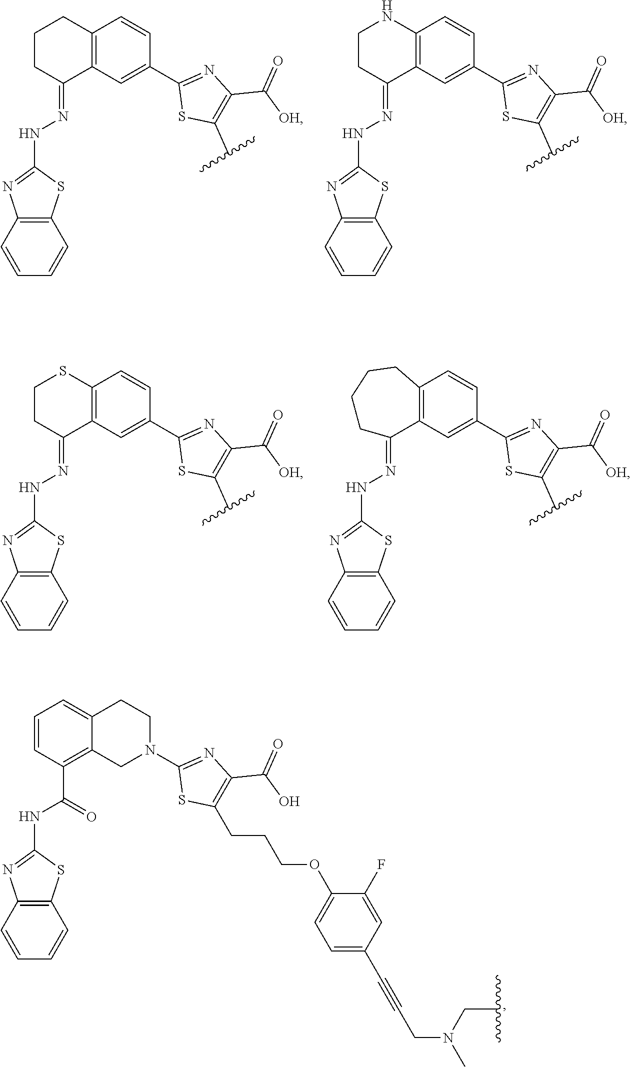 [SMILES: *c1sc(-c2ccc3c(c2)/C(=N/Nc2nc4ccccc4s2)CCC3)nc1C(=O)O ; *c1sc(-c2ccc3c(c2)/C(=N/Nc2nc4ccccc4s2)CCN3)nc1C(=O)O ; *c1sc(-c2ccc3c(c2)/C(=N/Nc2nc4ccccc4s2)CCS3)nc1C(=O)O ; *c1sc(-c2ccc3c(c2)/C(=N/Nc2nc4ccccc4s2)CCCC3)nc1C(=O)O ; *CN(C)CC#Cc1ccc(OCCCc2sc(N3CCc4cccc(C(=O)Nc5nc6ccccc6s5)c4C3)nc2C(=O)O)c(F)c1]

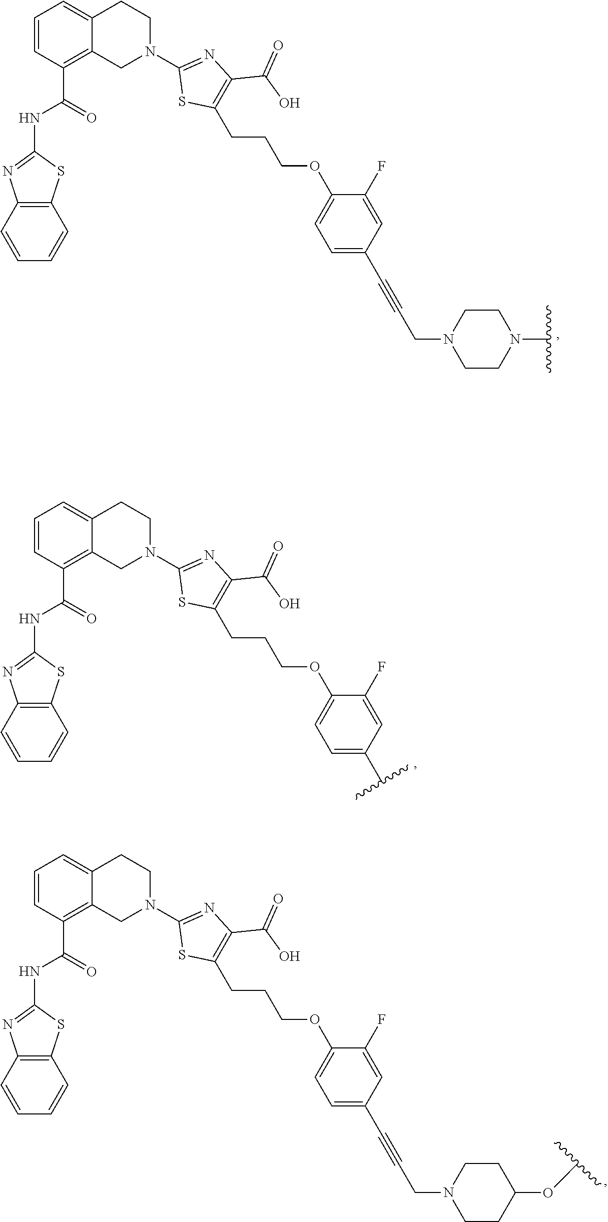 [SMILES: *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1]

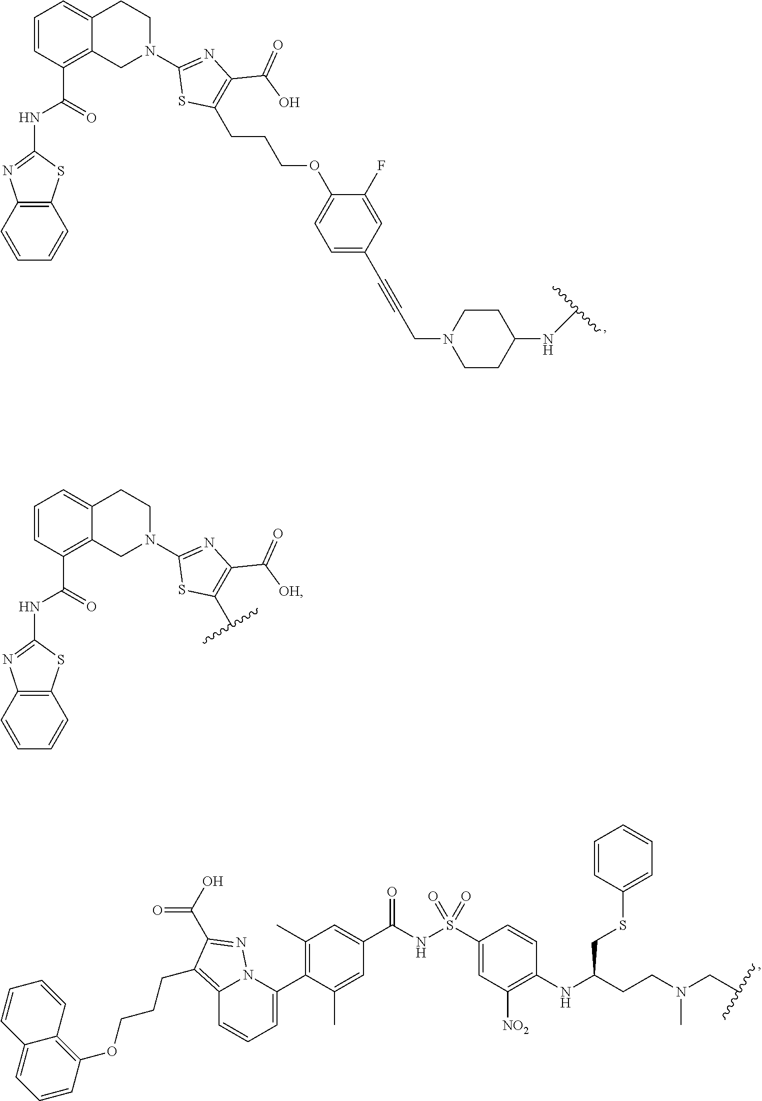 [SMILES: *NC1CCN(CC#Cc2ccc(OCCCc3sc(N4CCc5cccc(C(=O)Nc6nc7ccccc7s6)c5C4)nc3C(=O)O)c(F)c2)CC1 ; *c1sc(N2CCc3cccc(C(=O)Nc4nc5ccccc5s4)c3C2)nc1C(=O)O ; *CN(C)CC[C@H](CSc1ccccc1)Nc1ccc(S(=O)(=O)NC(=O)c2cc(C)c(-c3cccc4c(CCCOc5cccc6ccccc56)c(C(=O)O)nn34)c(C)c2)cc1[N+](=O)[O-]]

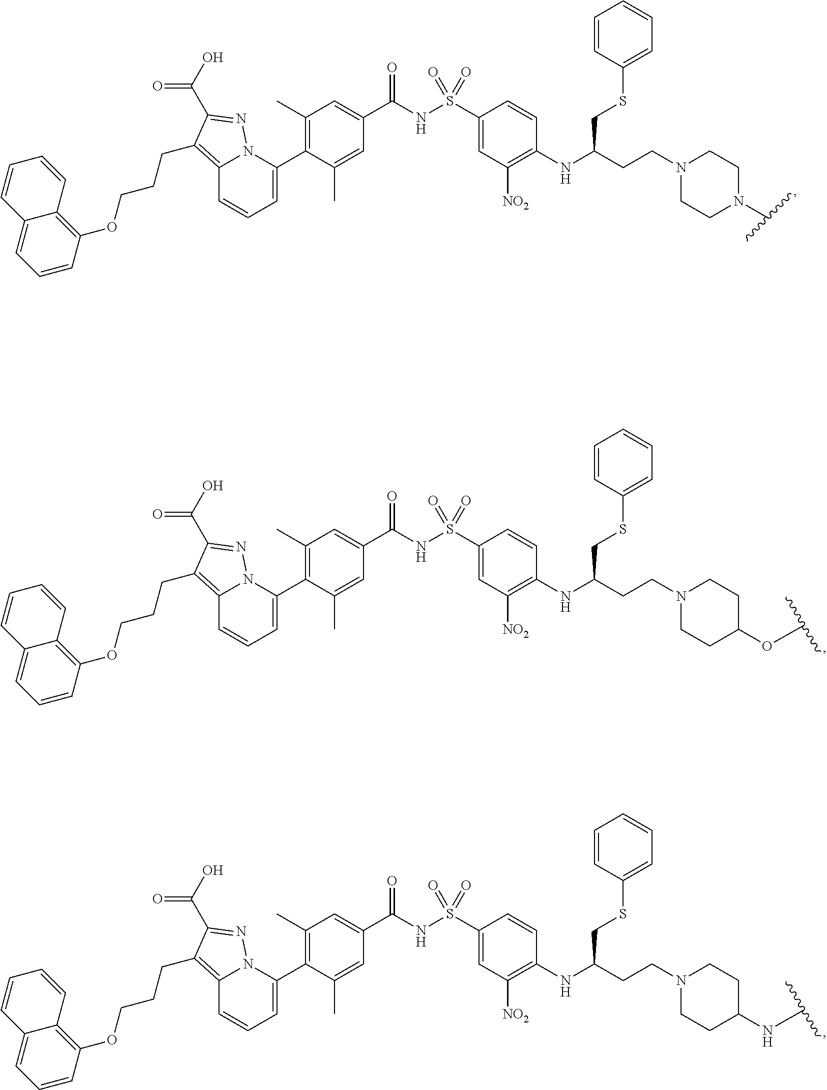 [SMILES: *N1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *OC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *NC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1]

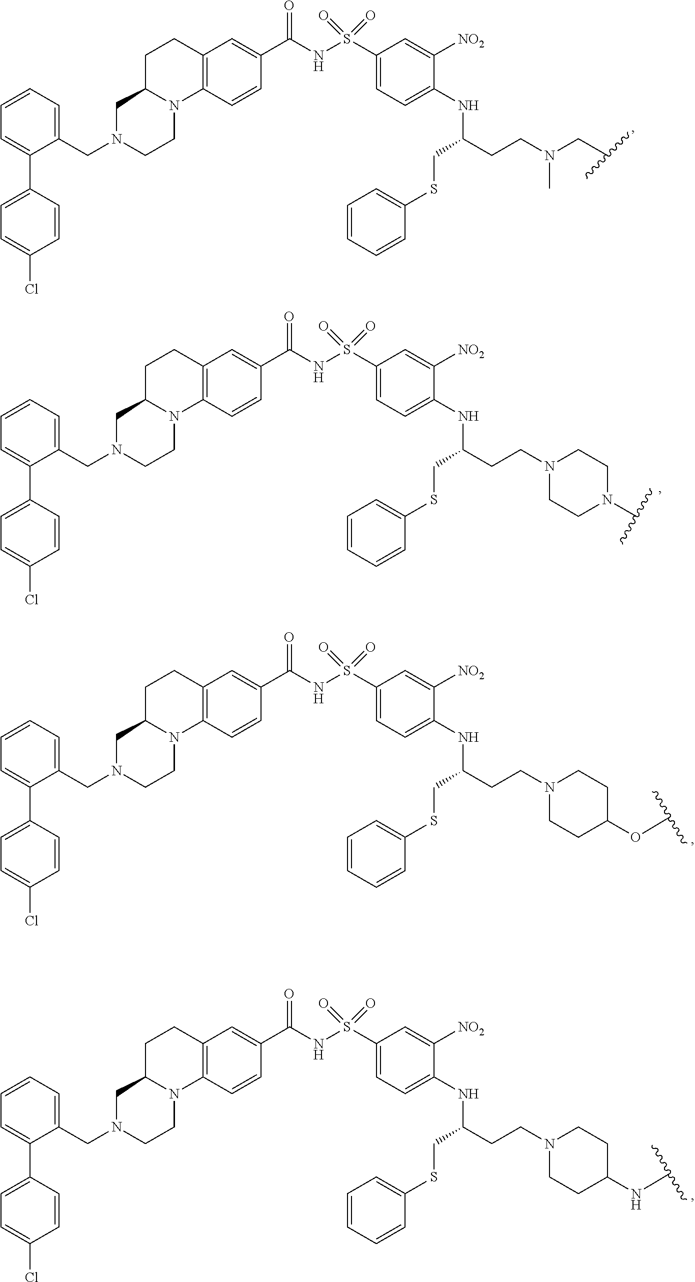 [SMILES: *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1]

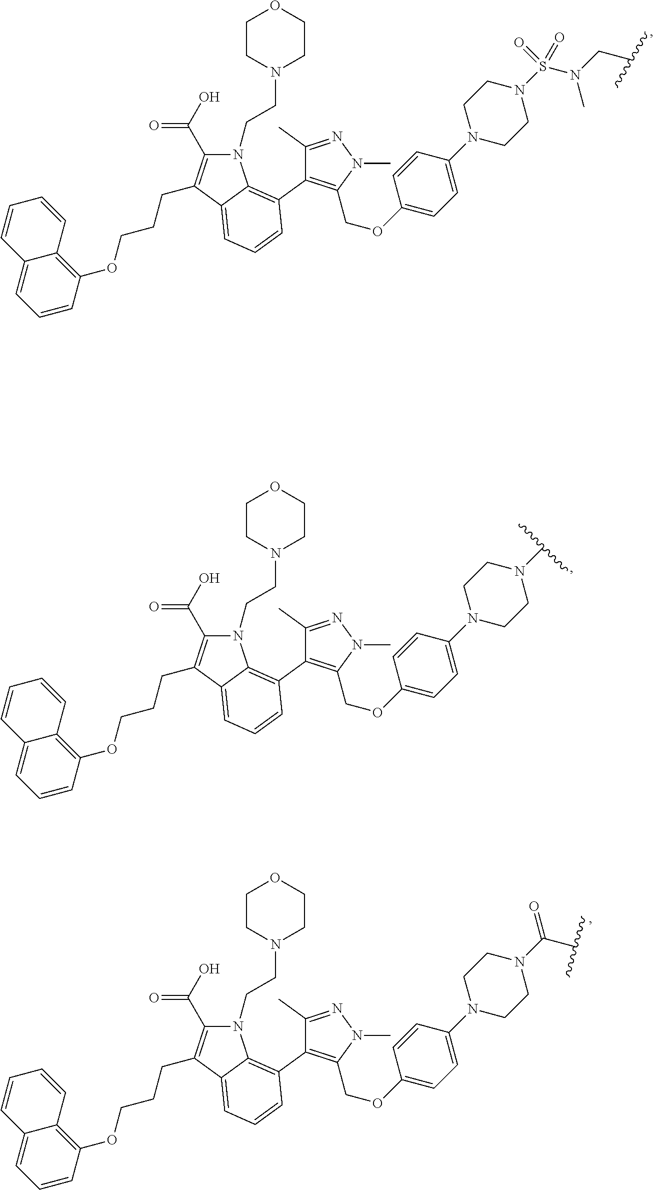 [SMILES: *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1]

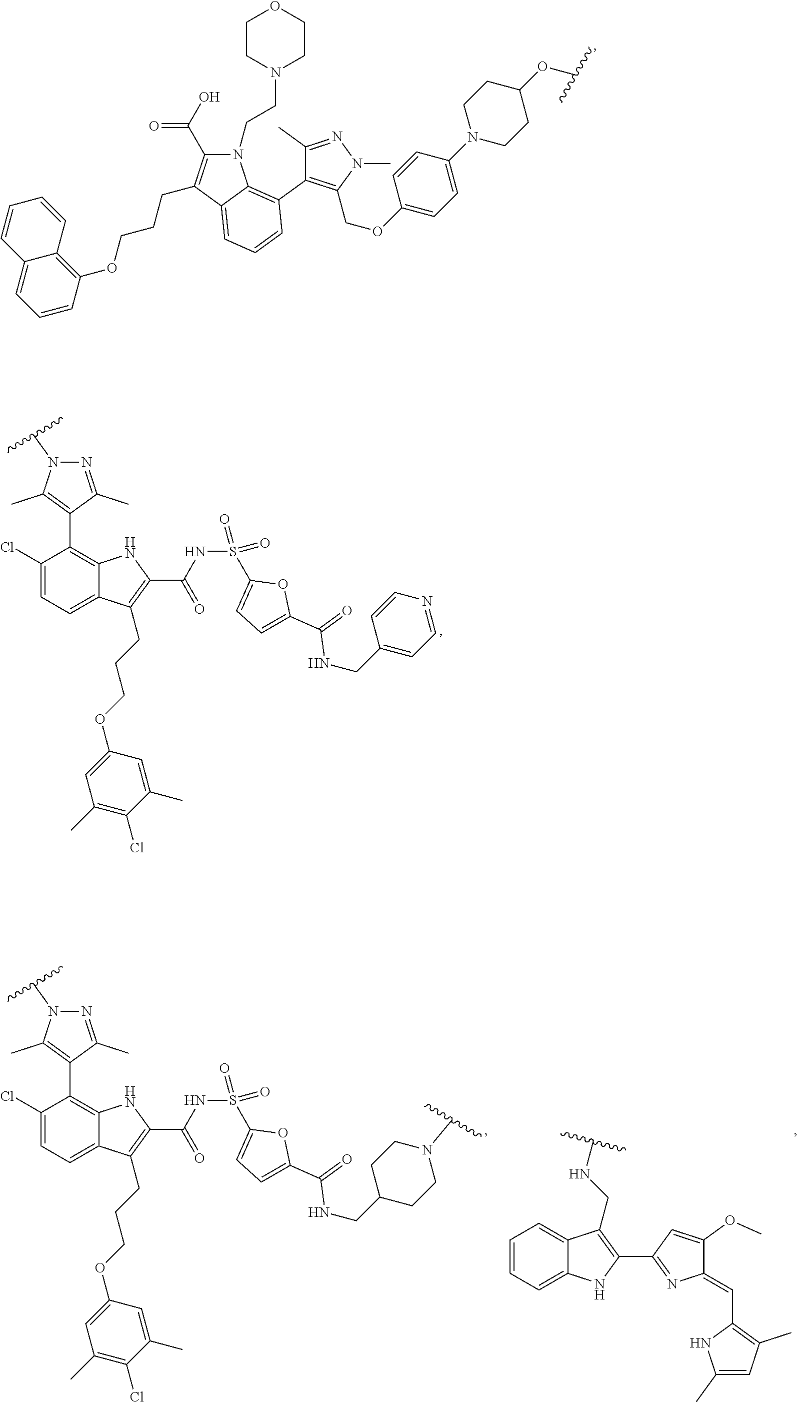 [SMILES: *OC1CCN(c2ccc(OCc3c(-c4cccc5c(CCCOc6cccc7ccccc67)c(C(=O)O)n(CCN6CCOCC6)c45)c(C)nn3C)cc2)CC1 ; *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(*)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12]

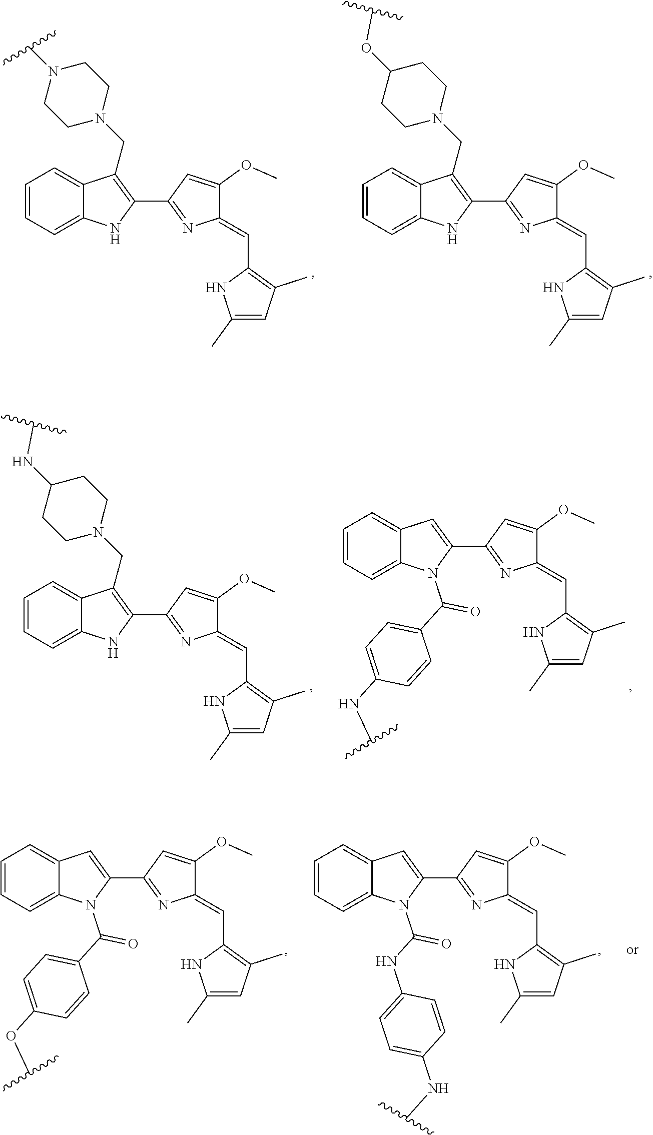 [SMILES: *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1 ; *NC1CCN(Cc2c(C3=N/C(=C\c4[nH]c(C)cc4C)C(OC)=C3)[nH]c3ccccc23)CC1 ; *Nc1ccc(C(=O)n2c(C3=N/C(=C\c4[nH]c(C)cc4C)C(OC)=C3)cc3ccccc32)cc1 ; *Oc1ccc(C(=O)n2c(C3=N/C(=C\c4[nH]c(C)cc4C)C(OC)=C3)cc3ccccc32)cc1 ; *Nc1ccc(NC(=O)n2c(C3=N/C(=C\c4[nH]c(C)cc4C)C(OC)=C3)cc3ccccc32)cc1]

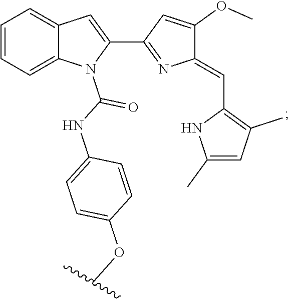 [SMILES: *Oc1ccc(NC(=O)n2c(C3=N/C(=C\c4[nH]c(C)cc4C)C(OC)=C3)cc3ccccc32)cc1]

R³ may be absent, an unsubstituted C₁-C₆ alkyl, or a substituted or unsubstituted C₃-C₆ ketone; A may be absent, a bond, or a substituted or unsubstituted C₁-C₆ heterocyclic group; n may be 0 to 3; R⁴ may be a bond or a substituted or unsubstituted C₁-C₁₀ alkyl; and R² may be

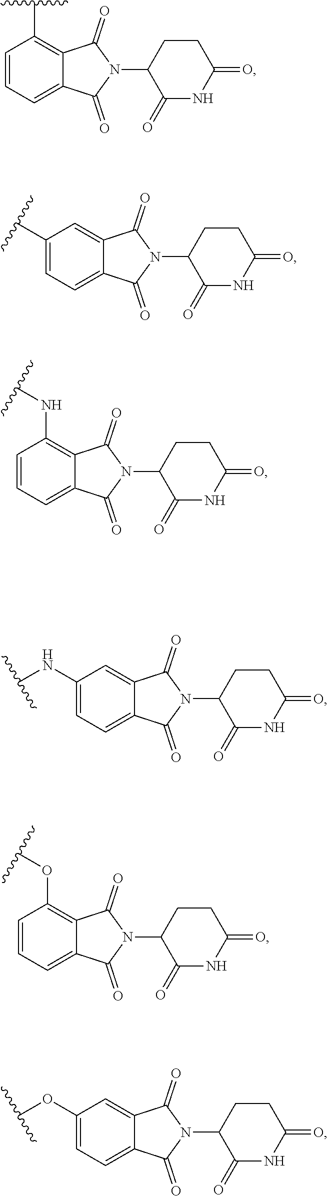 [SMILES: *c1cccc2c1C(=O)N(C1CCC(=O)NC1=O)C2=O ; *c1ccc2c(c1)C(=O)N(C1CCC(=O)NC1=O)C2=O ; *Nc1cccc2c1C(=O)N(C1CCC(=O)NC1=O)C2=O ; *Nc1ccc2c(c1)C(=O)N(C1CCC(=O)NC1=O)C2=O ; *Oc1cccc2c1C(=O)N(C1CCC(=O)NC1=O)C2=O ; *Oc1ccc2c(c1)C(=O)N(C1CCC(=O)NC1=O)C2=O]

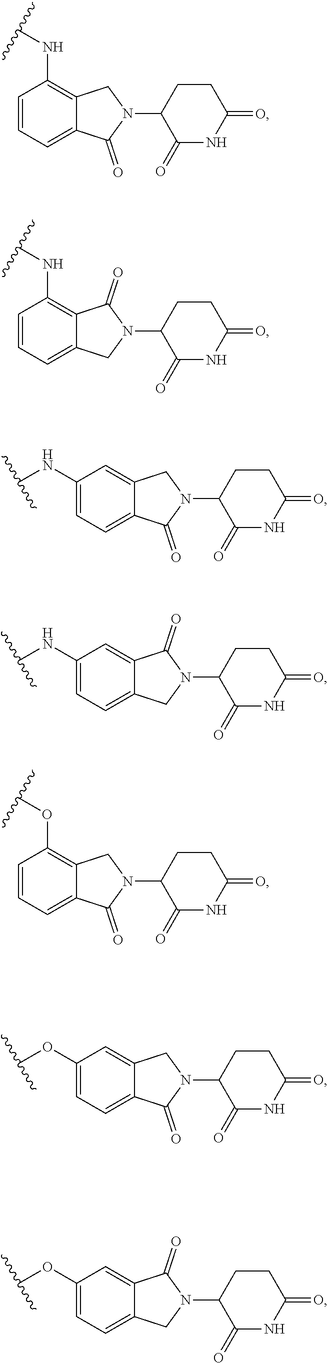

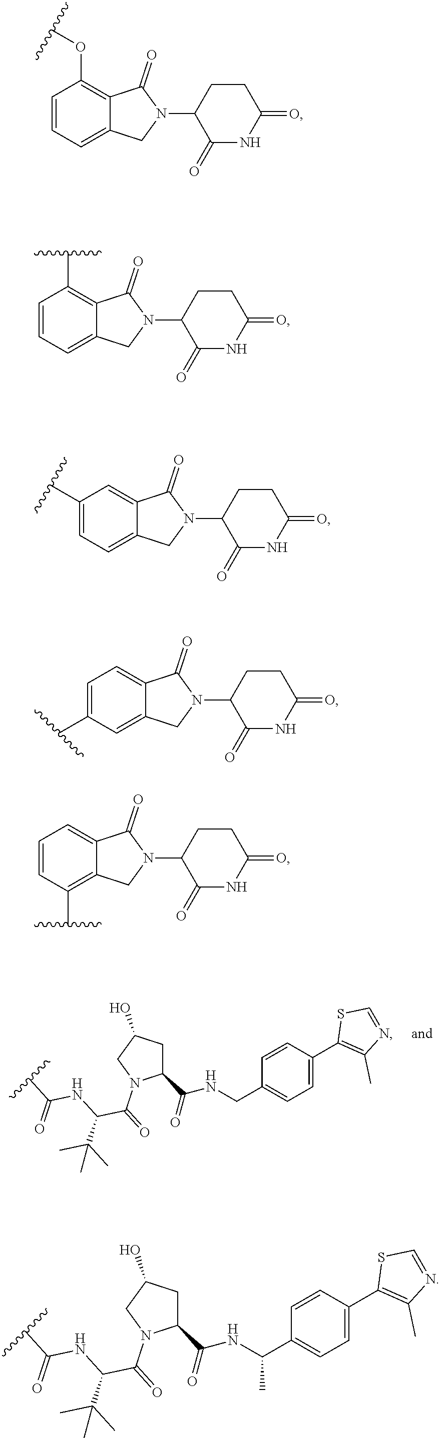

In another embodiment, a compound of the disclosure comprises Formula (II), wherein R¹ may be

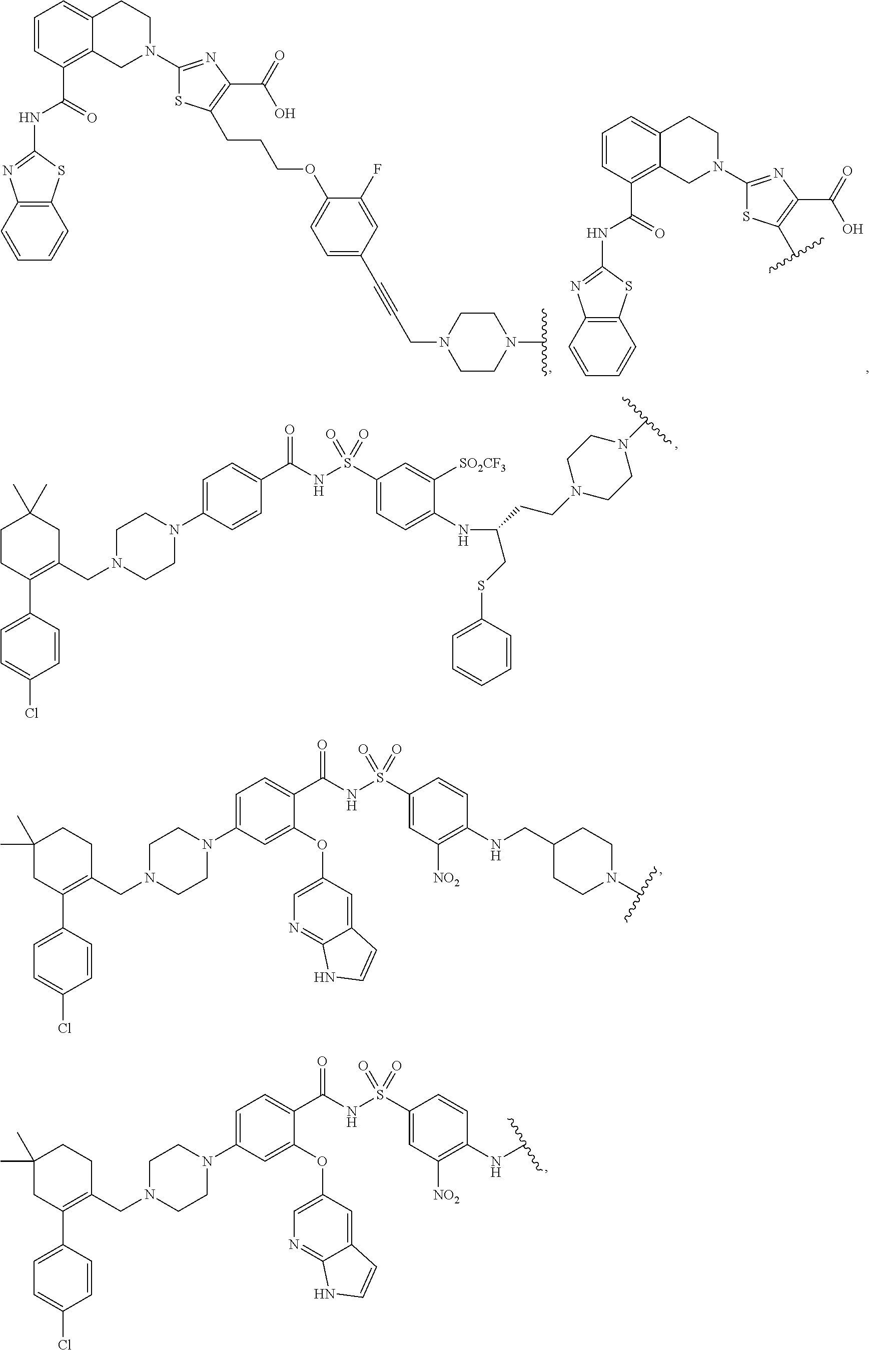

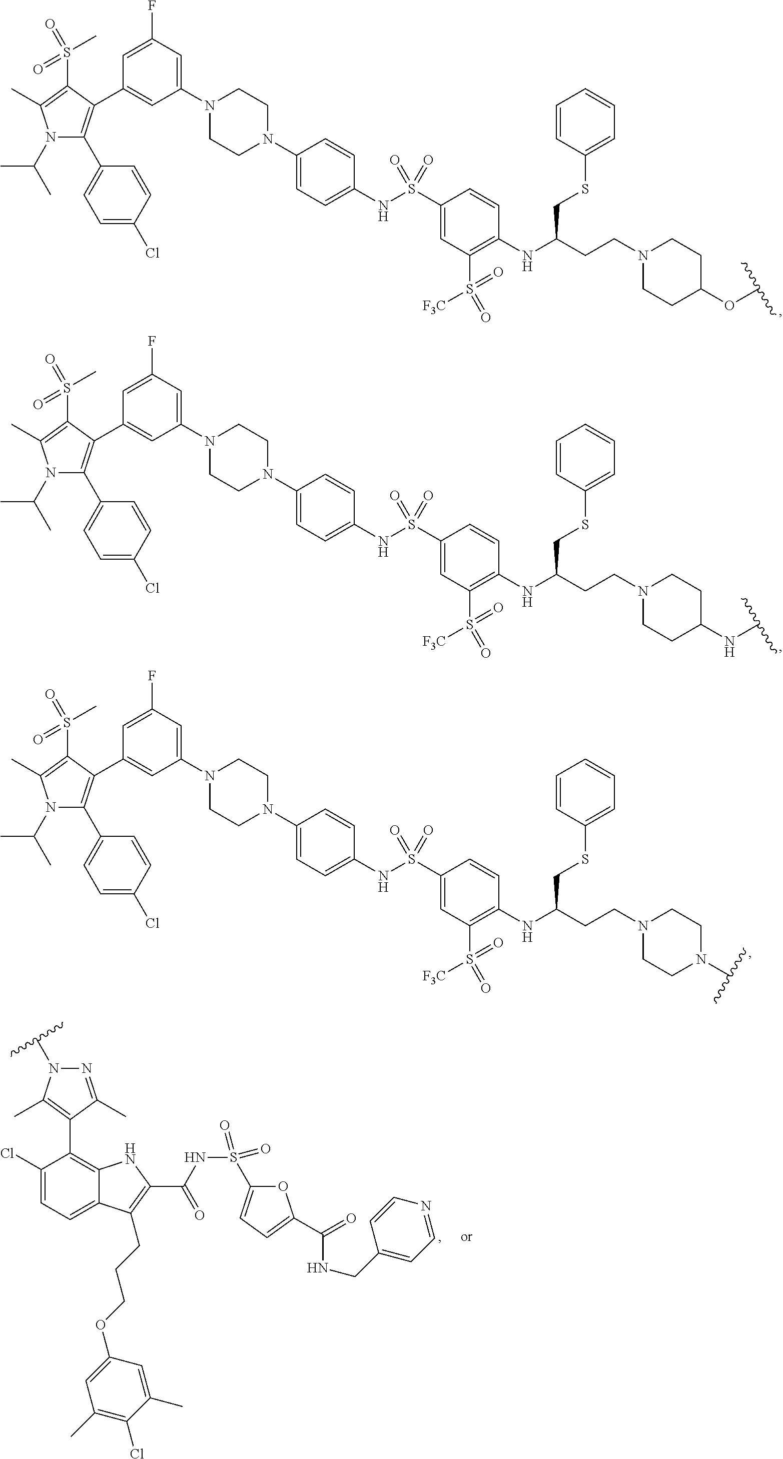

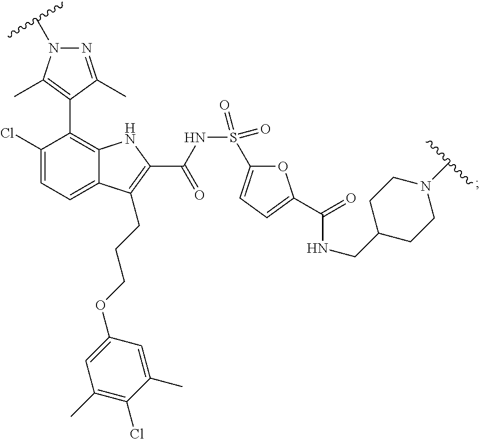 [SMILES: *N1CCC(CNC(=O)c2ccc(S(=O)(=O)NC(=O)c3[nH]c4c(-c5c(C)nn(*)c5C)c(Cl)ccc4c3CCCOc3cc(C)c(Cl)c(C)c3)o2)CC1]

R³ may be absent, an unsubstituted C₁-C₆ alkyl, or a substituted or unsubstituted C₃-C₆ ketone; A may be absent, a bond, or a substituted or unsubstituted C₁-C₆ heterocyclic group; n may be 0 to 3, R⁴ may be a bond or a substituted or unsubstituted C₁-C₁₀ alkyl; and R² may be

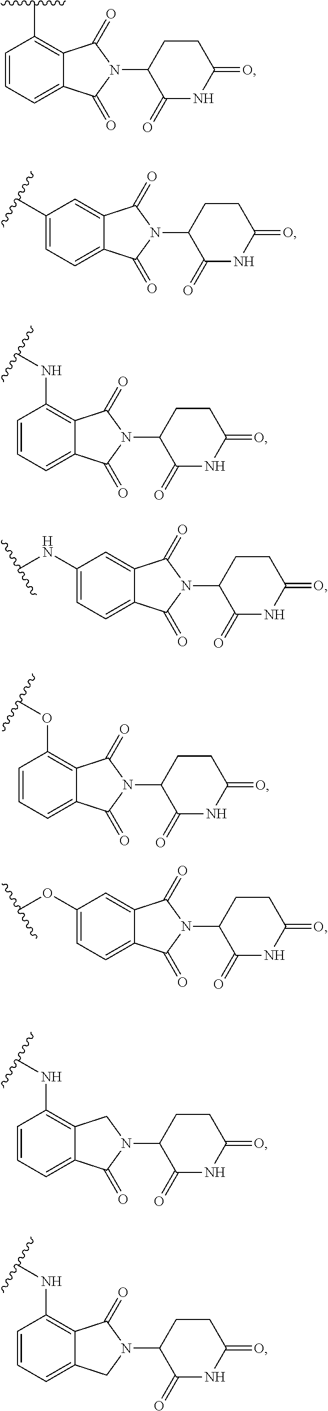

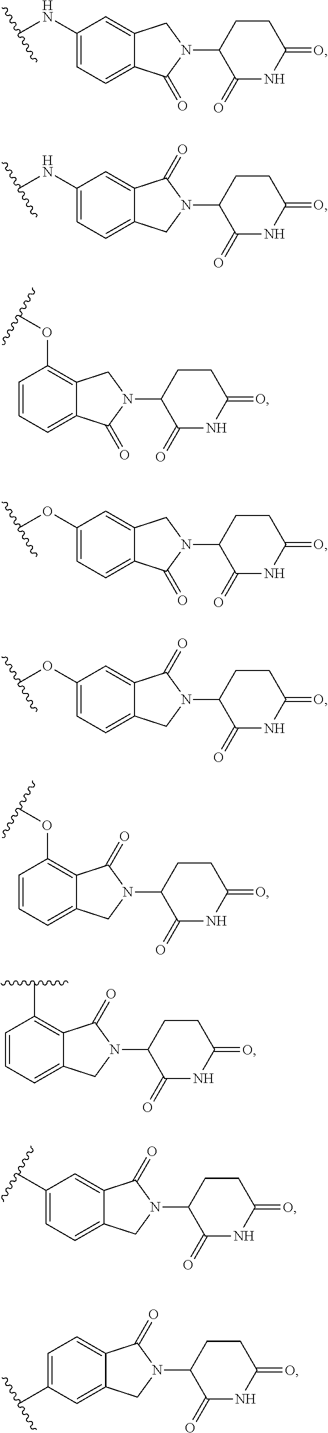

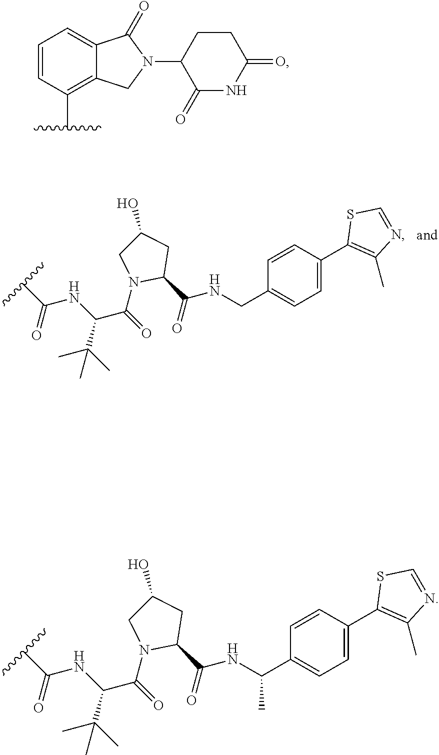 [SMILES: *c1cccc2c1CN(C1CCC(=O)NC1=O)C2=O ; *C(=O)N[C@H](C(=O)N1C[C@H](O)C[C@H]1C(=O)NCc1ccc(-c2scnc2C)cc1)C(C)(C)C ; "and" ; *C(=O)N[C@H](C(=O)N1C[C@H](O)C[C@H]1C(=O)N[C@@H](C)c1ccc(-c2scnc2C)cc1)C(C)(C)C]

In still another embodiment, a compound of the disclosure comprises Formula (II), wherein R¹ may be

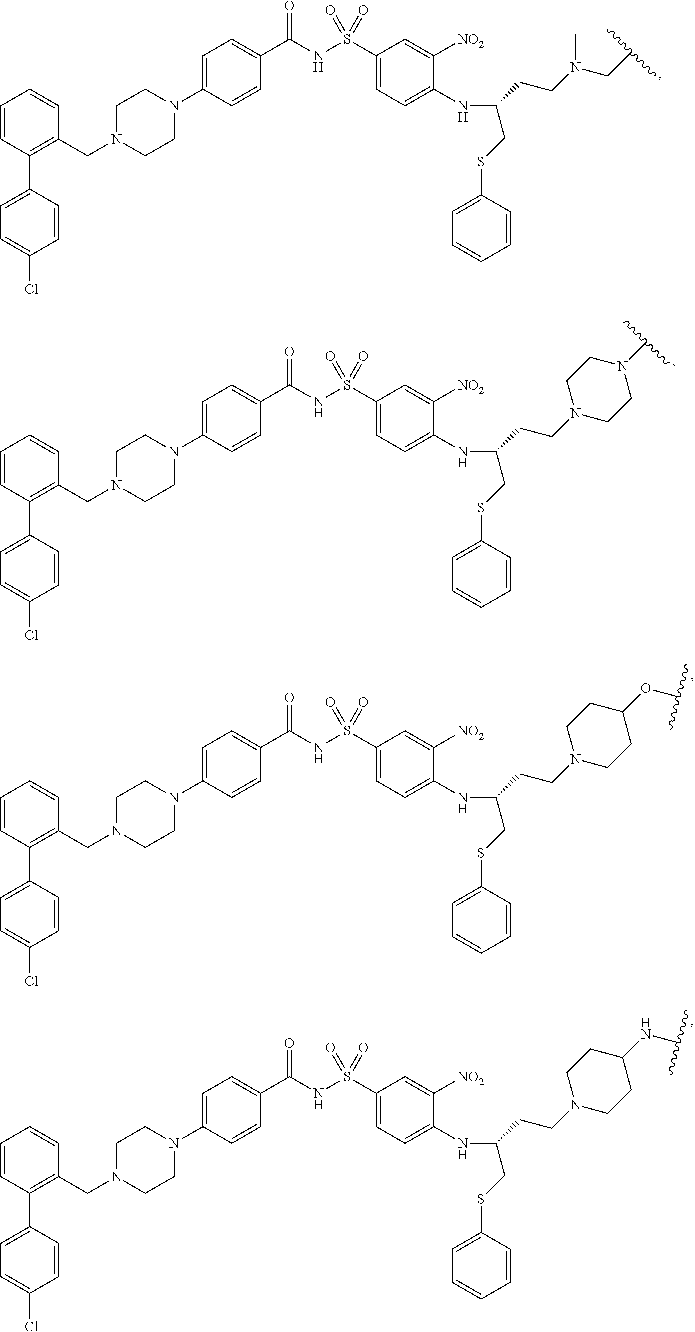 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

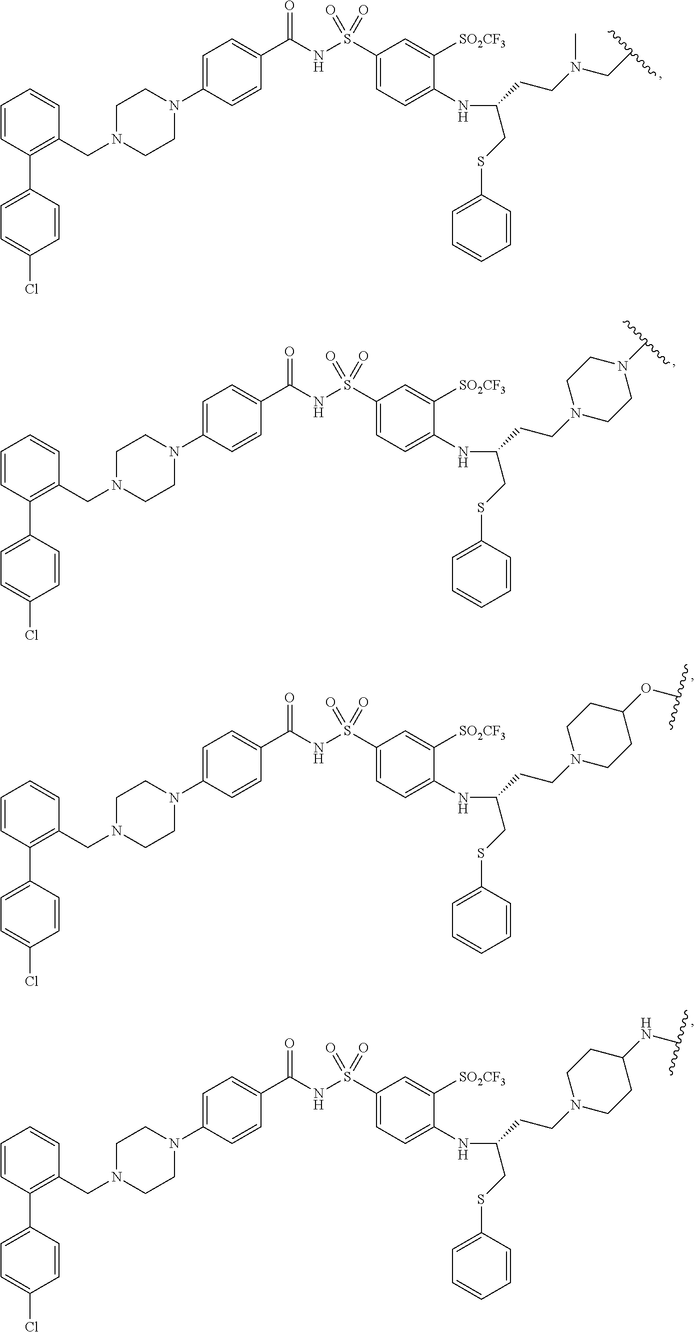

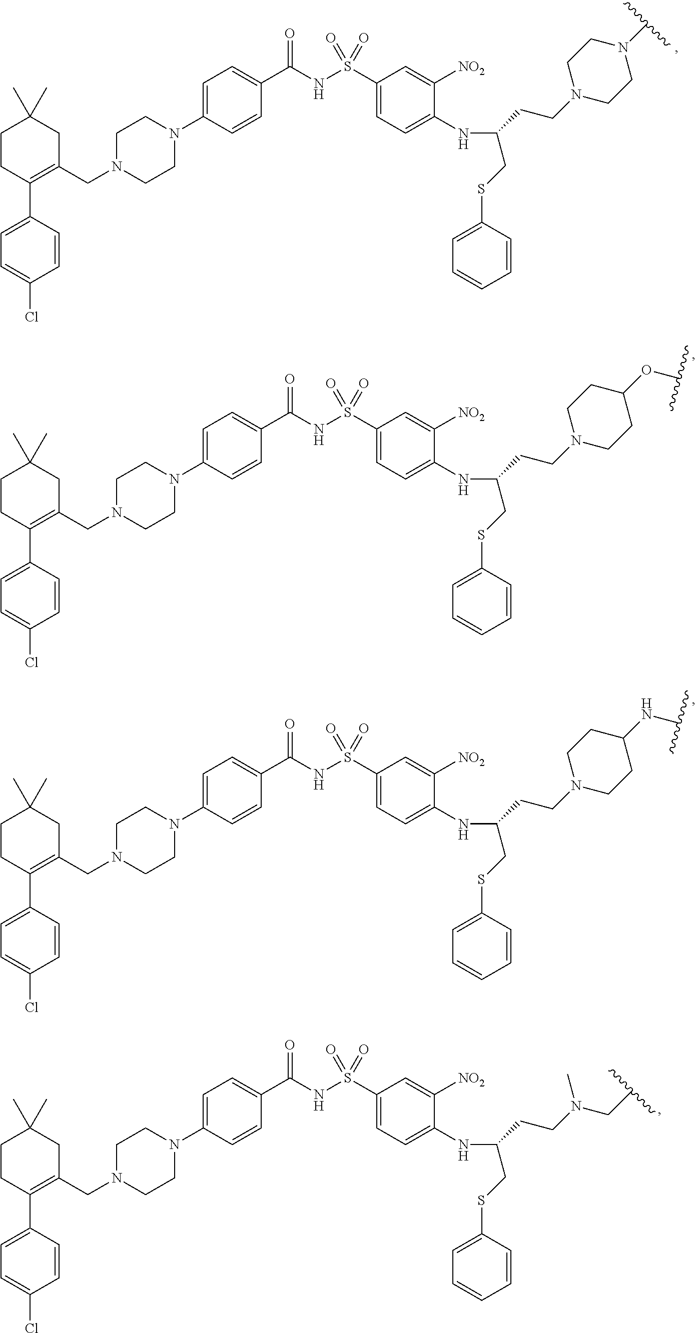 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

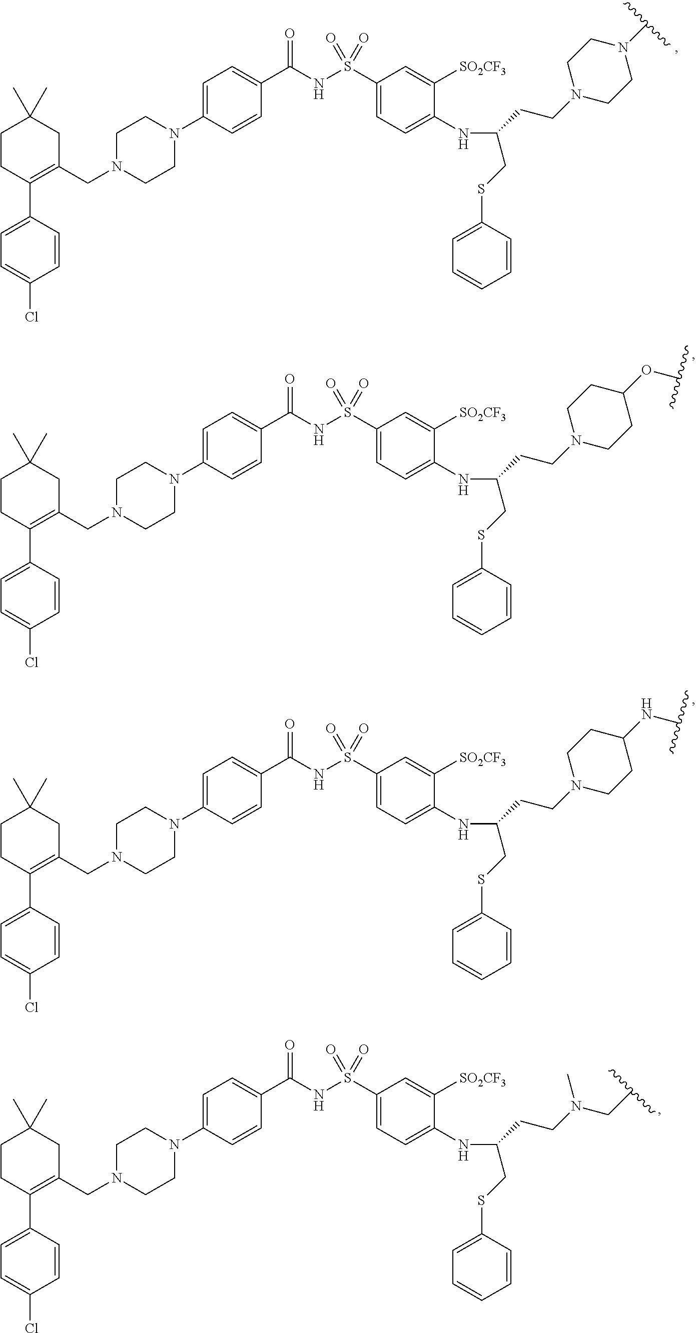

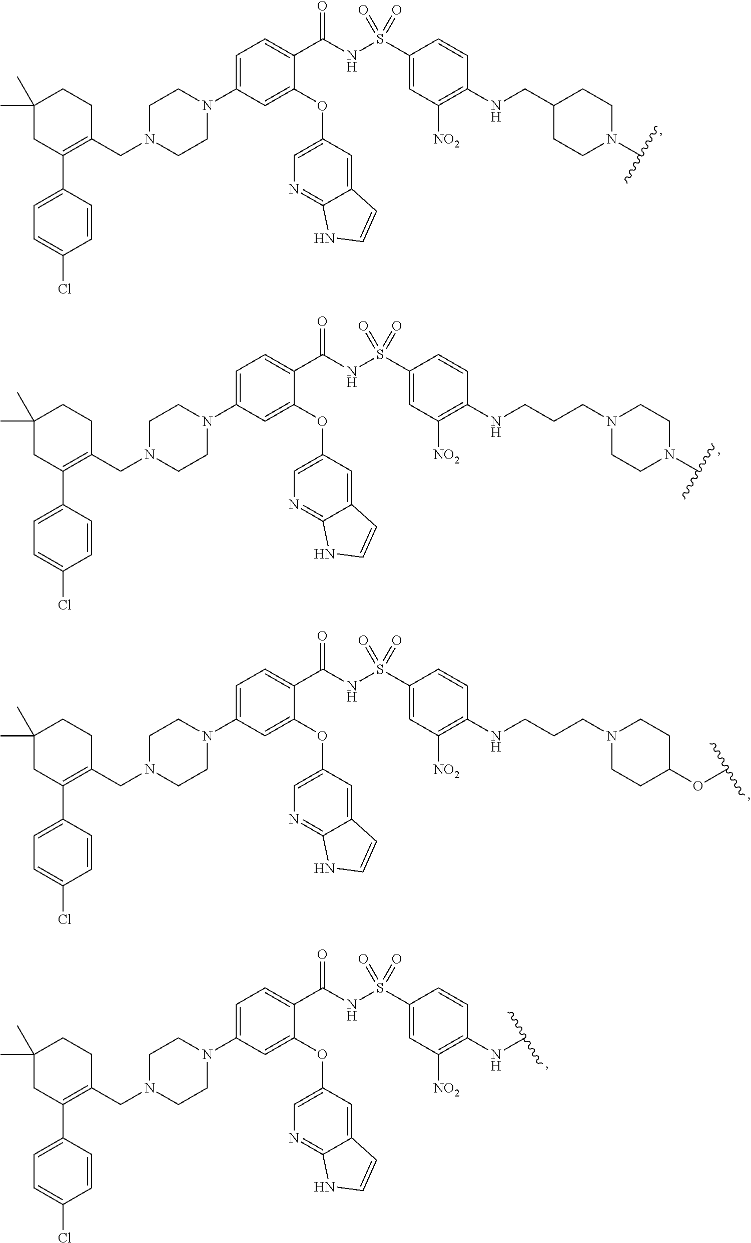 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *OC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

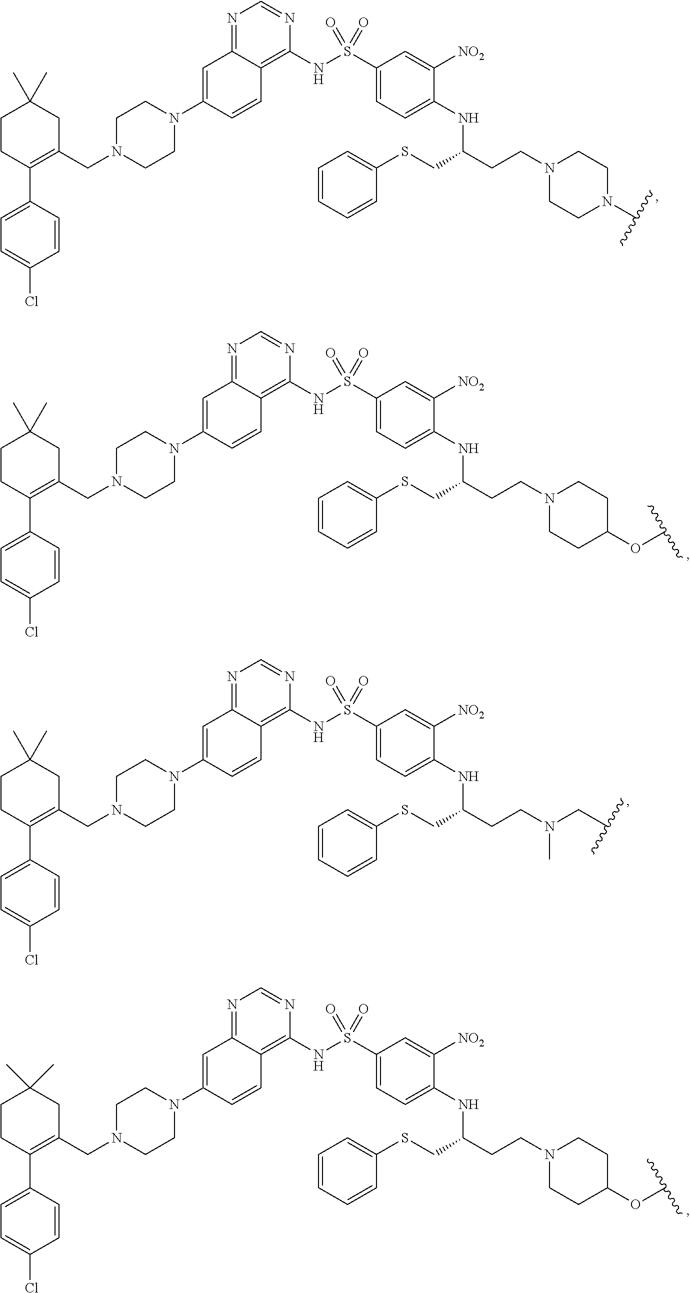 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

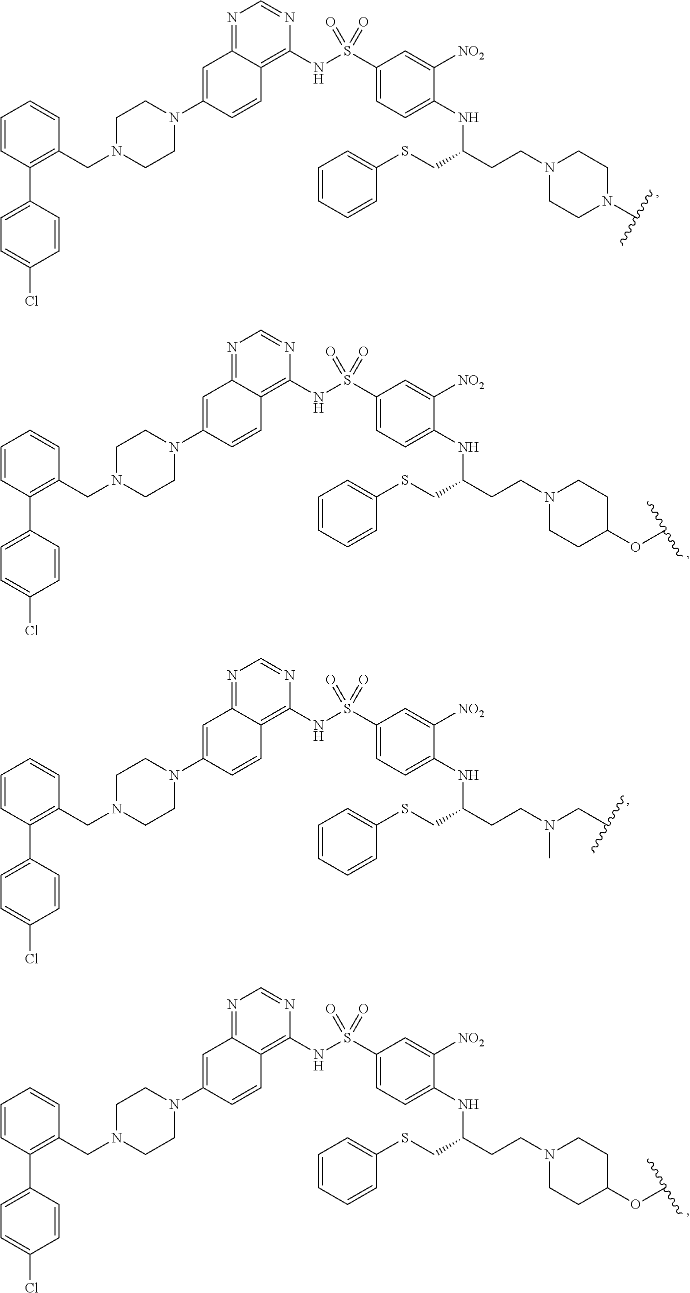 [SMILES: *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *CN(C)CC[C@H](CSc1ccccc1)Nc1ccc(S(=O)(=O)Nc2ncnc3cc(N4CCN(Cc5ccccc5-c5ccc(Cl)cc5)CC4)ccc23)cc1[N+](=O)[O-] ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

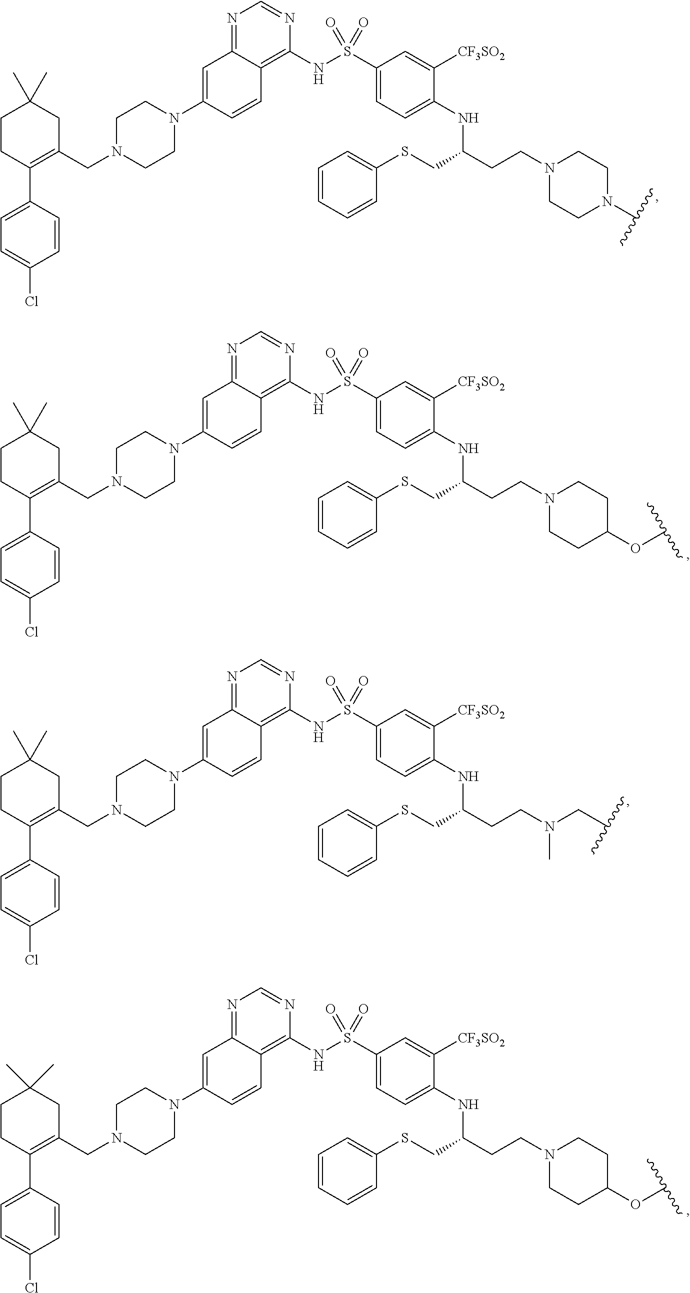

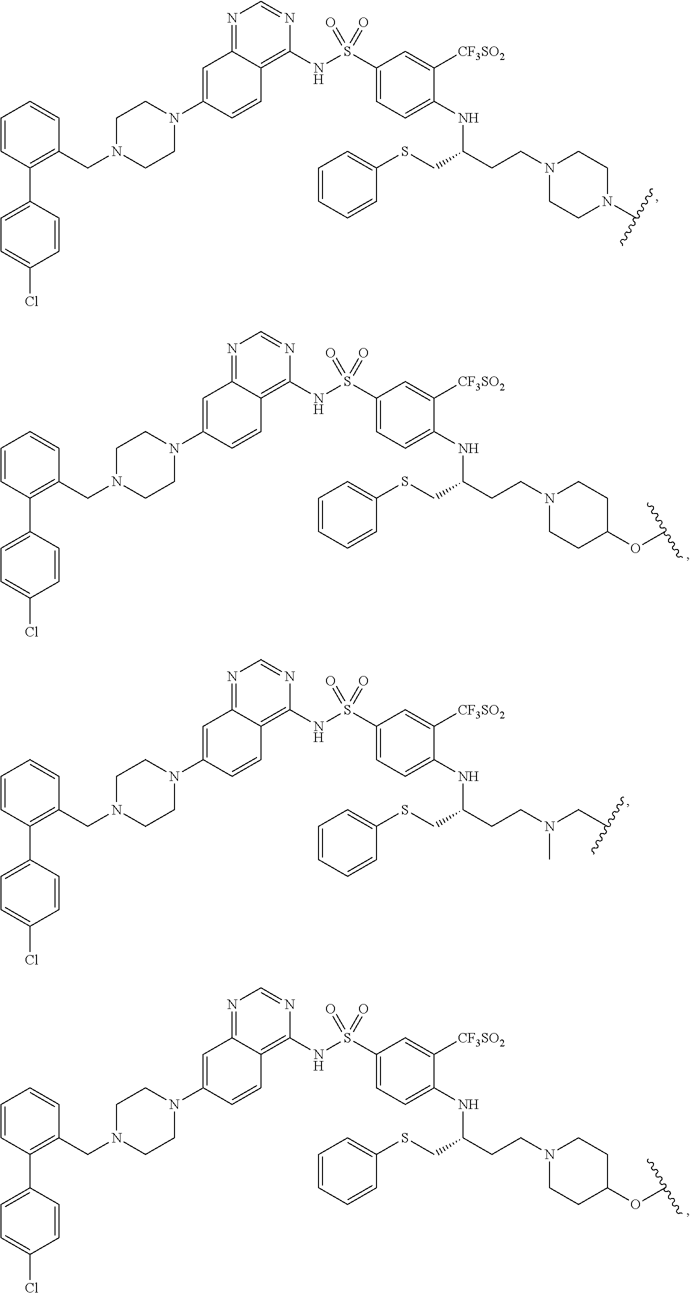

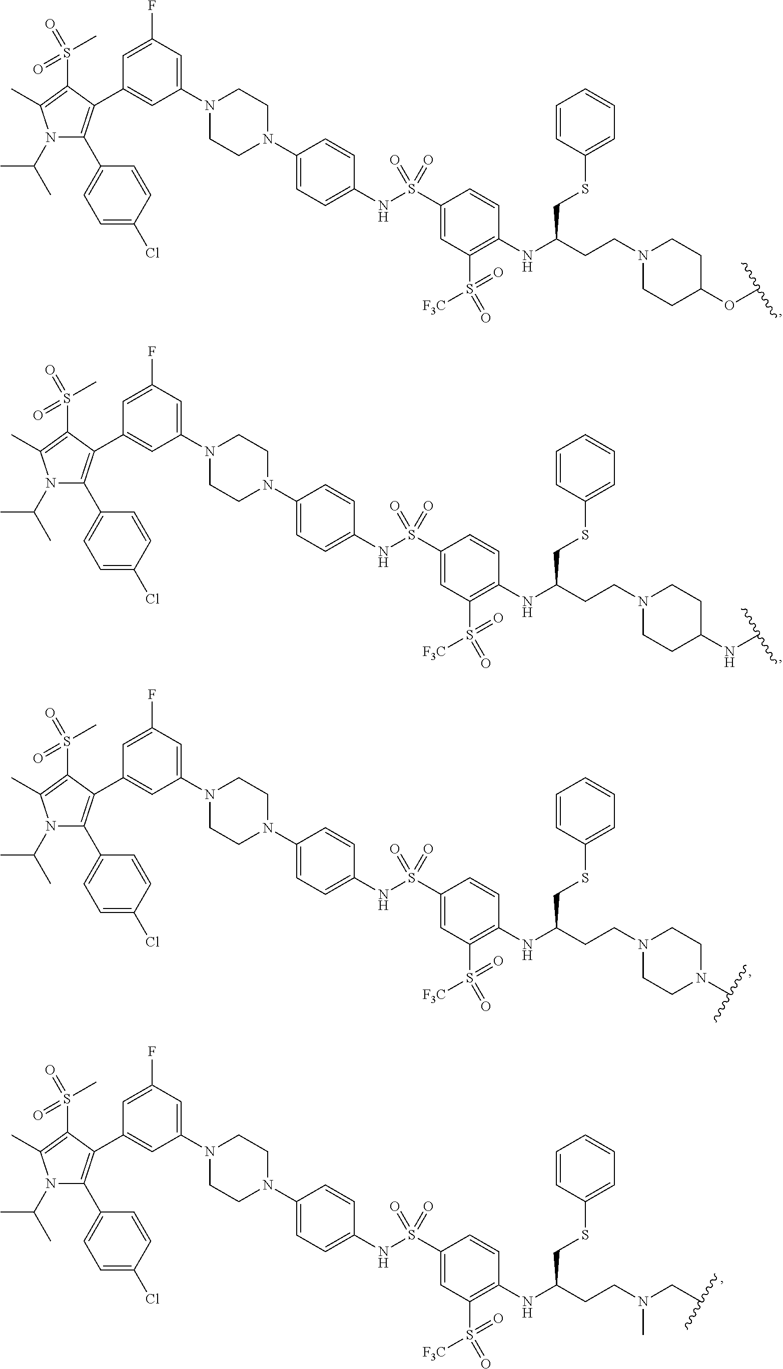

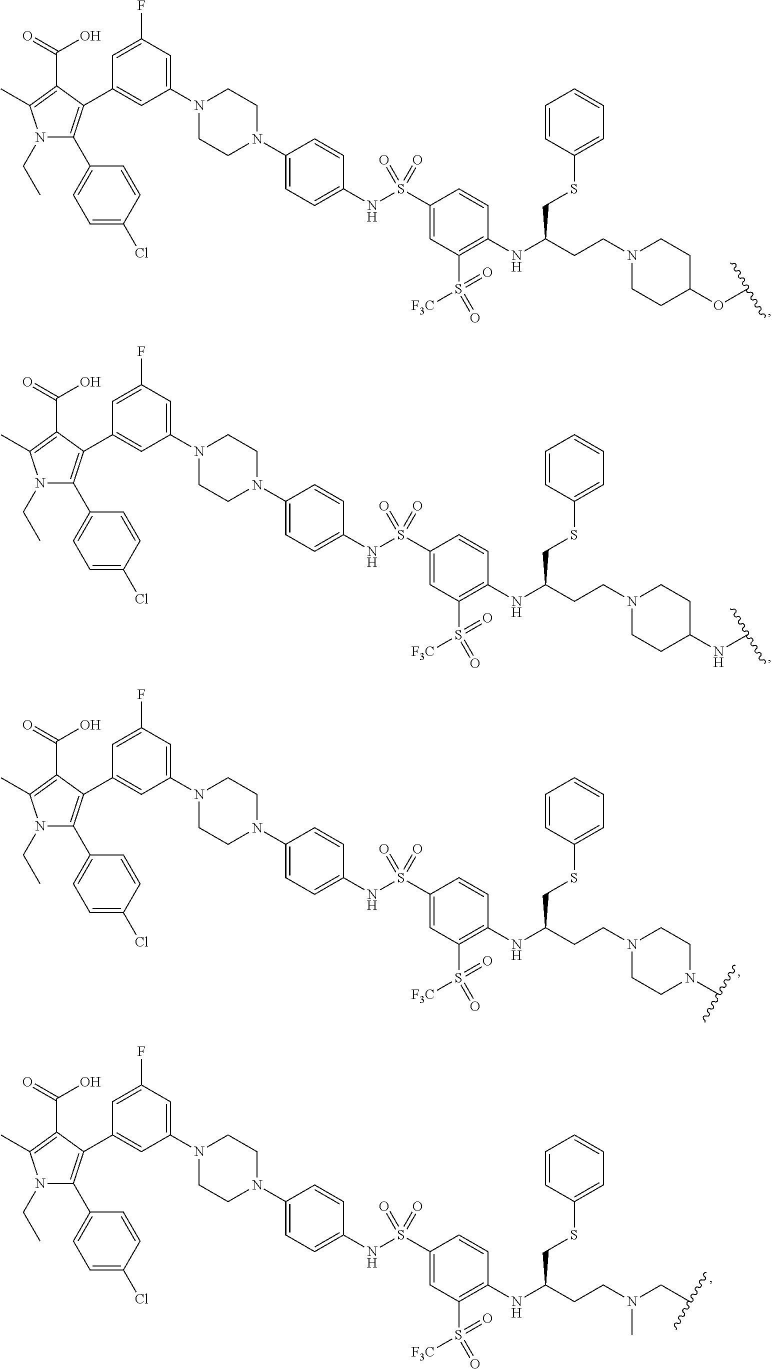

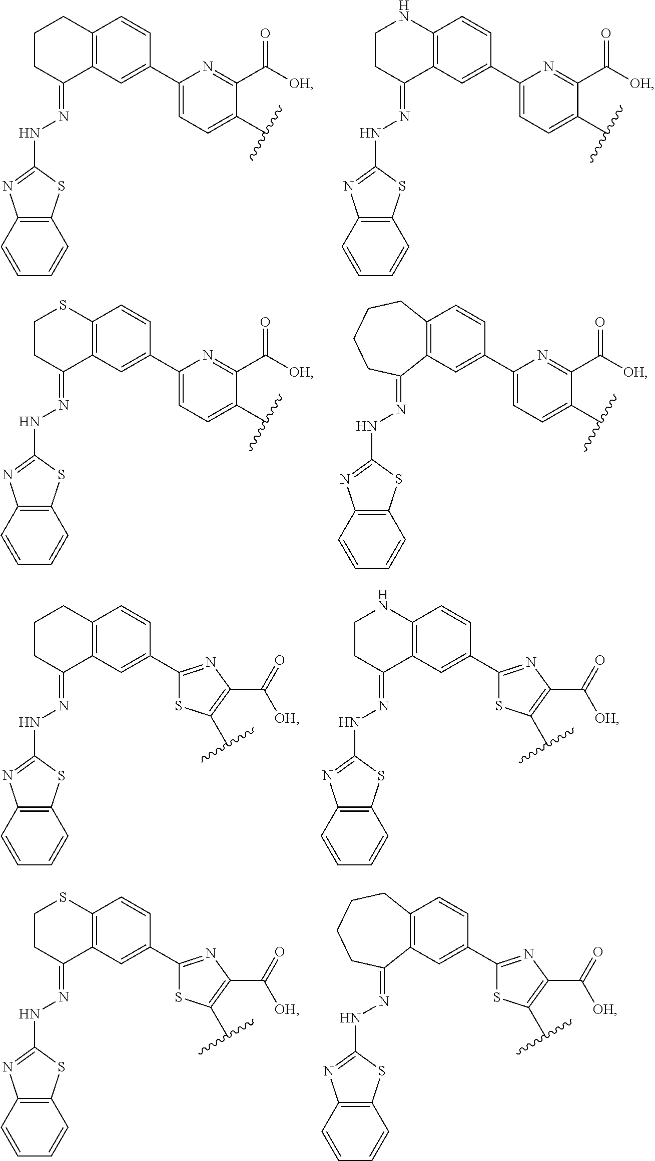

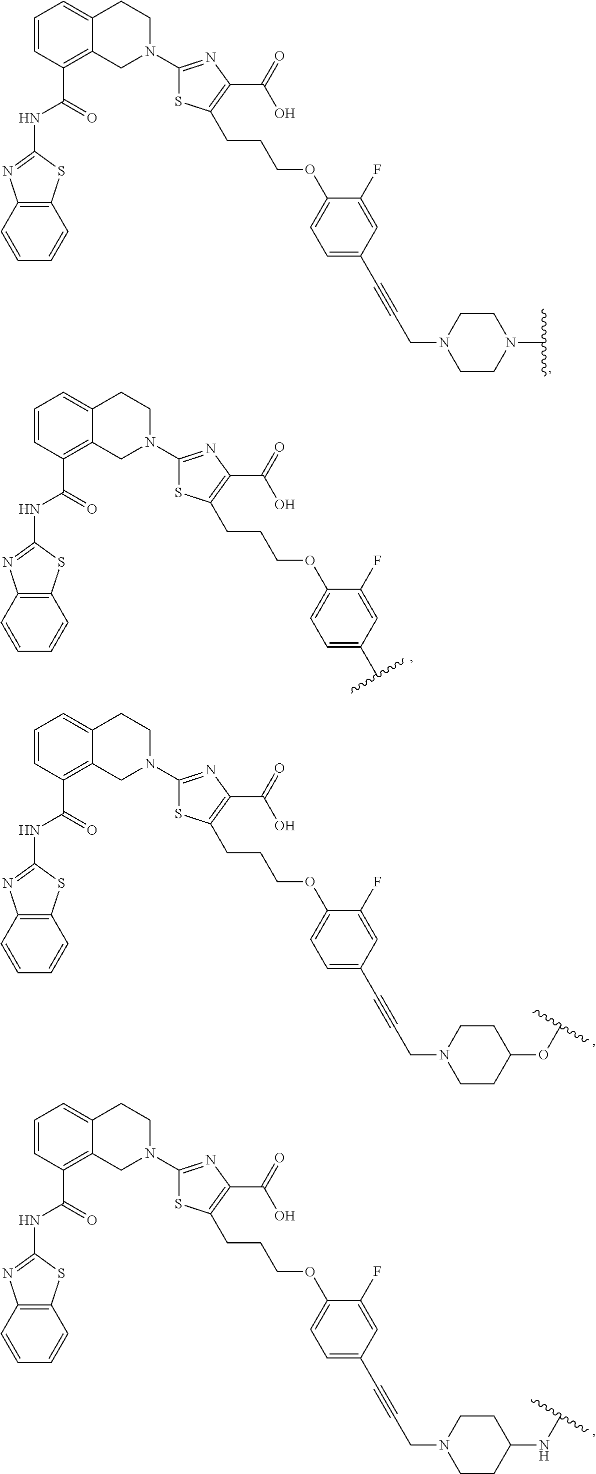 [SMILES: *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1 ; *NC1CCN(CC#Cc2ccc(OCCCc3sc(N4CCc5cccc(C(=O)Nc6nc7ccccc7s6)c5C4)nc3C(=O)O)c(F)c2)CC1]

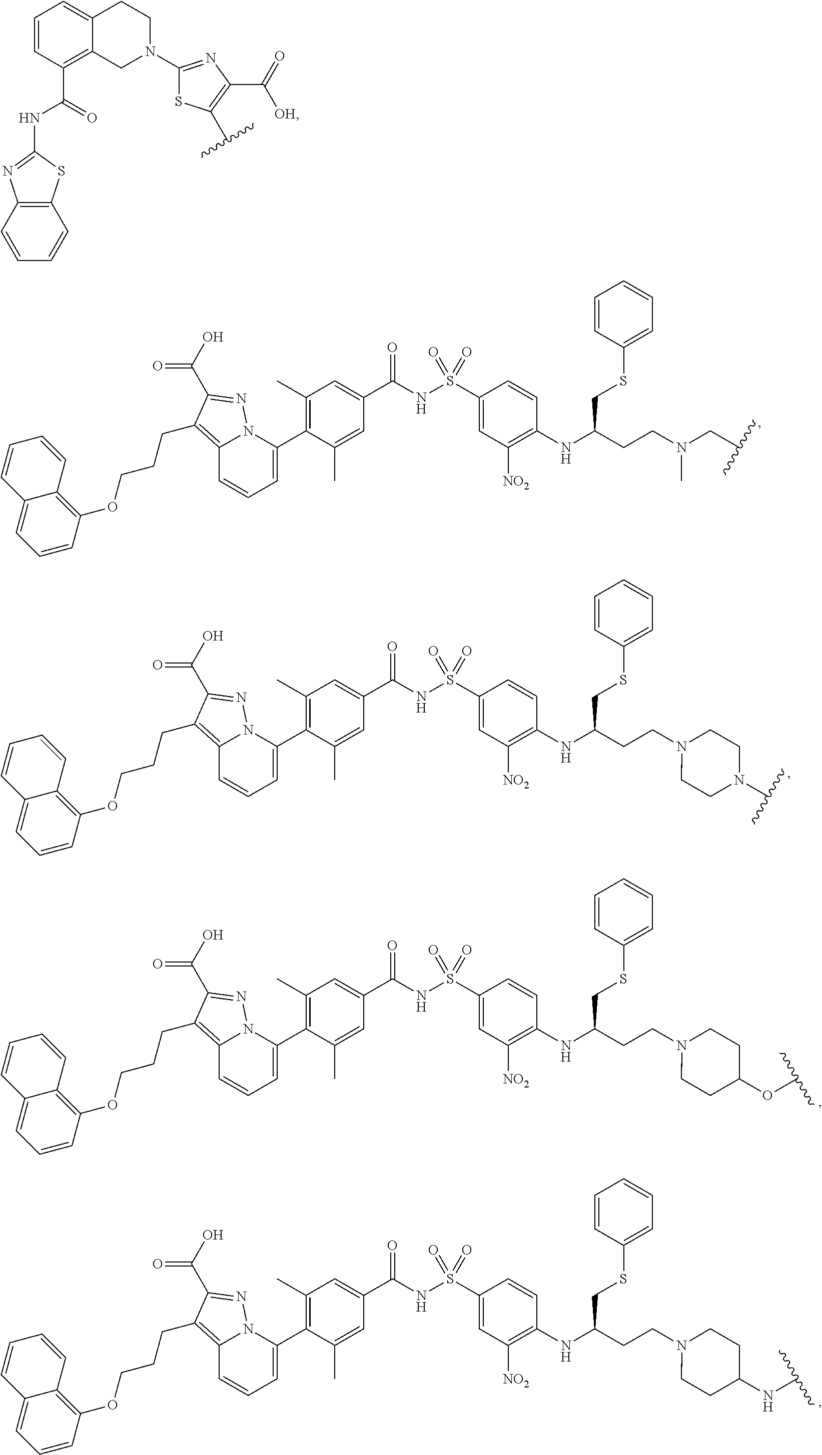

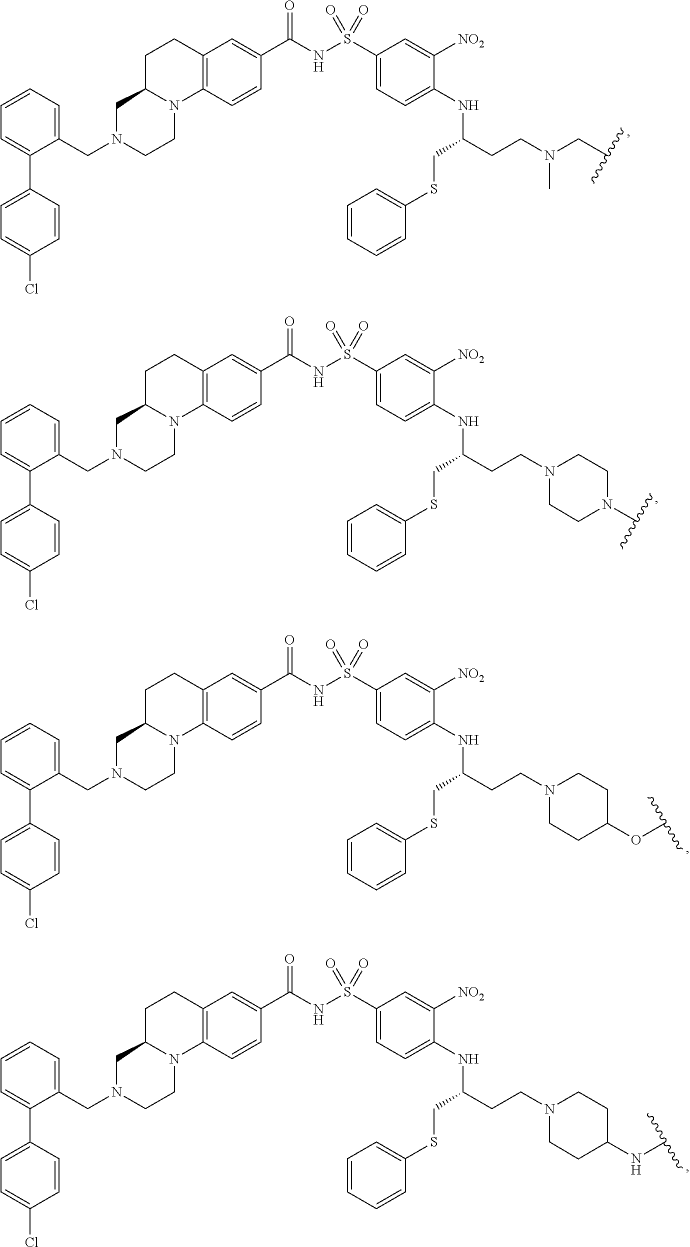 [SMILES: *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1]

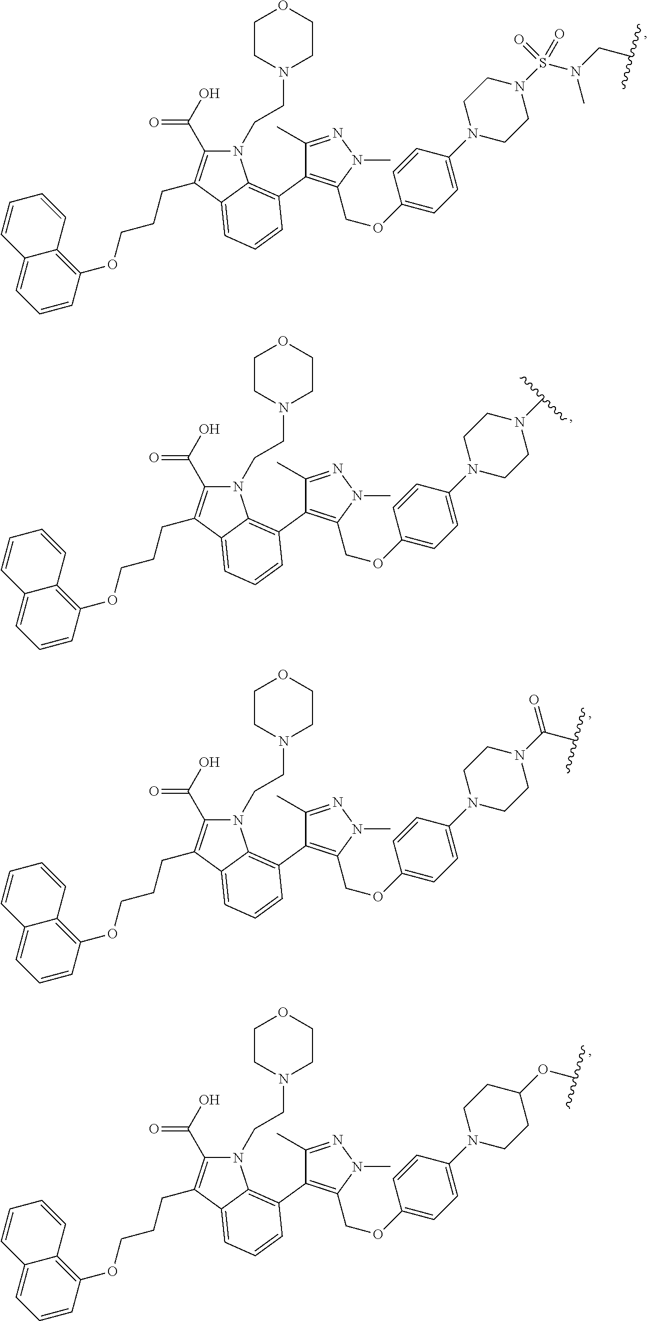 [SMILES: *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *OC1CCN(c2ccc(OCc3c(-c4cccc5c(CCCOc6cccc7ccccc67)c(C(=O)O)n(CCN6CCOCC6)c45)c(C)nn3C)cc2)CC1]

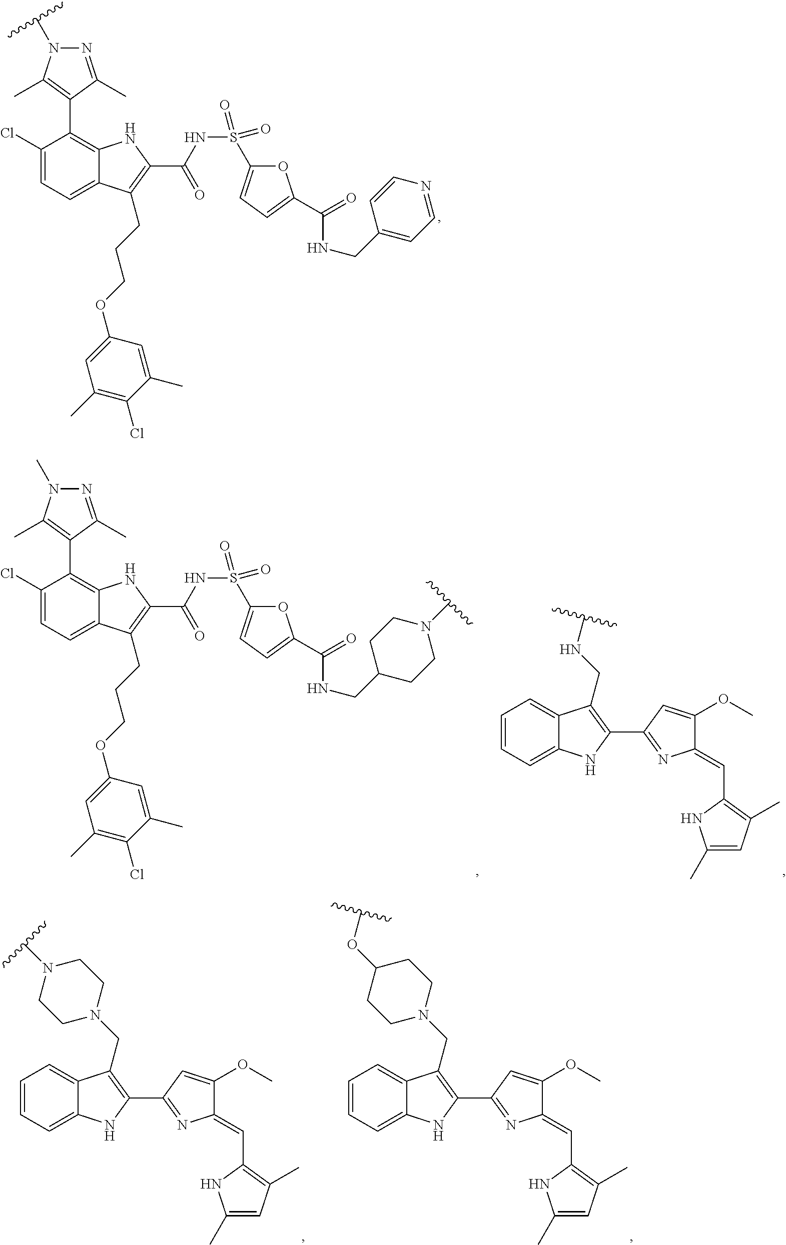 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12 ; *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1]

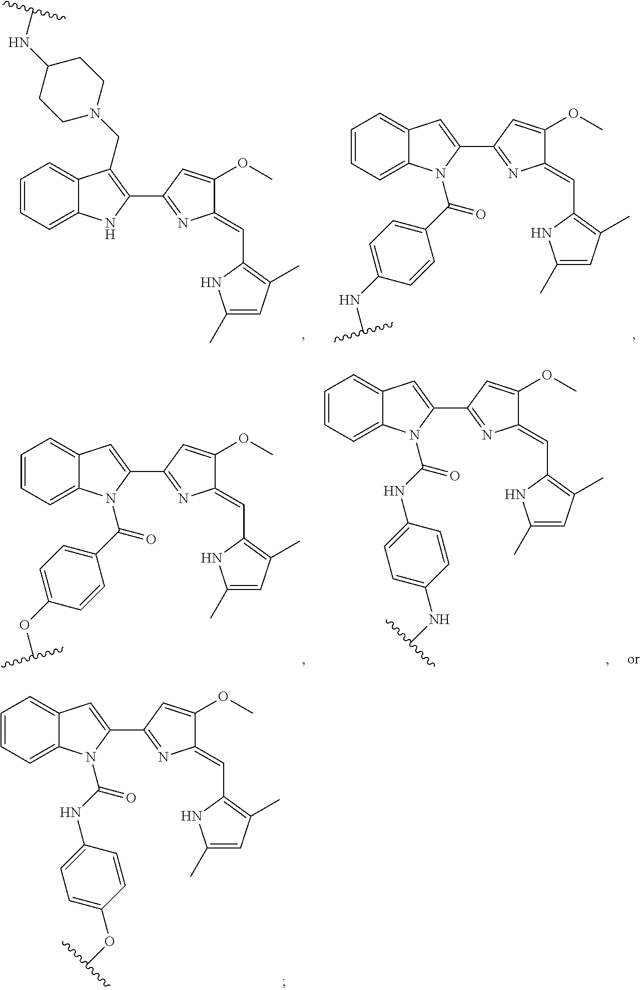

R³ may be absent, a bond, 2-pentanone, or an unsubstituted C₂-C₃ alkyl; B may be absent, a bond, or a substituted or unsubstituted C₁-C₆ heterocyclic group; n may be 0 to 3; R⁴ may be a bond or a substituted or unsubstituted C₁-C₁₀ alkyl; and R² may be

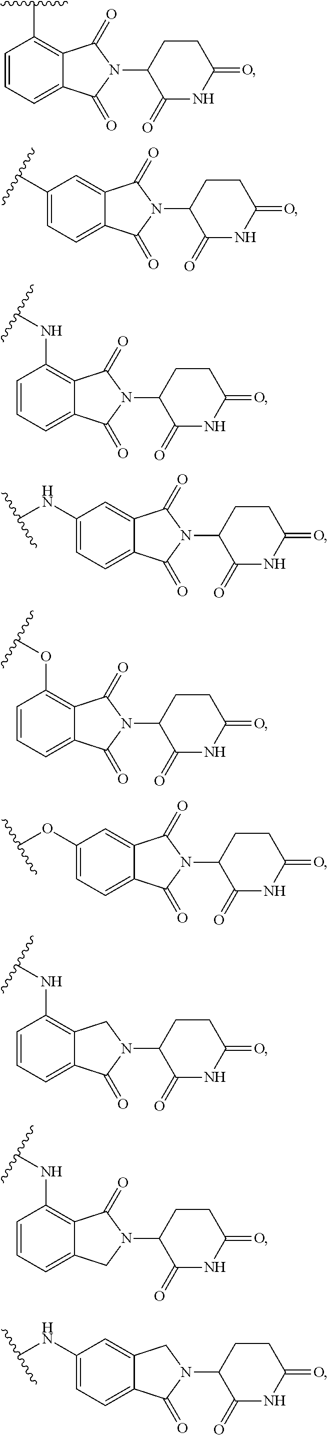

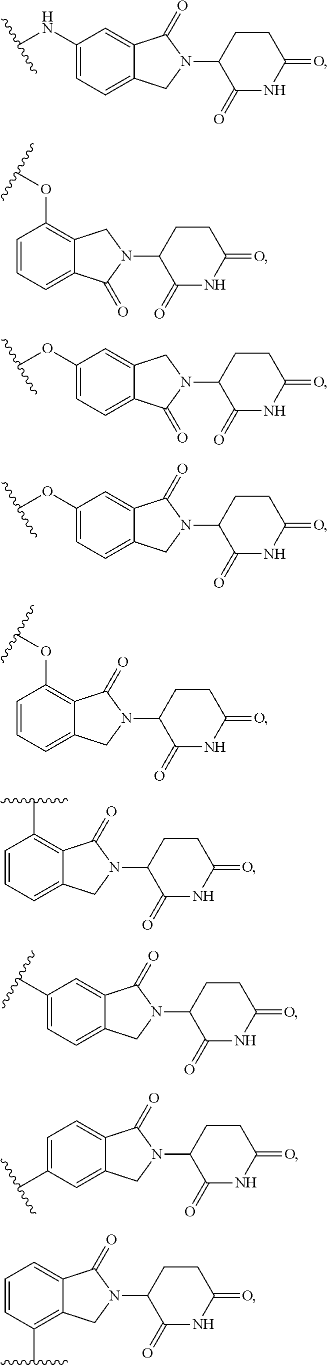

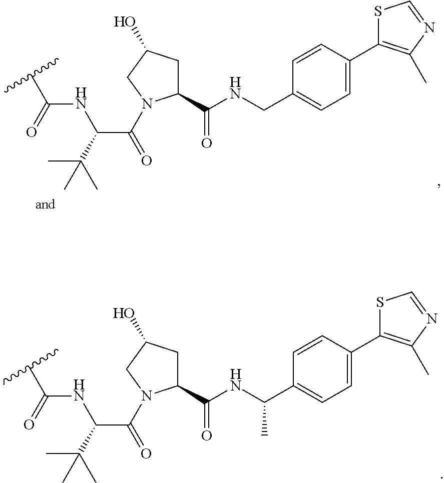 [SMILES: *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

In still another embodiment, a compound of the disclosure comprises Formula (II), wherein R¹ may be

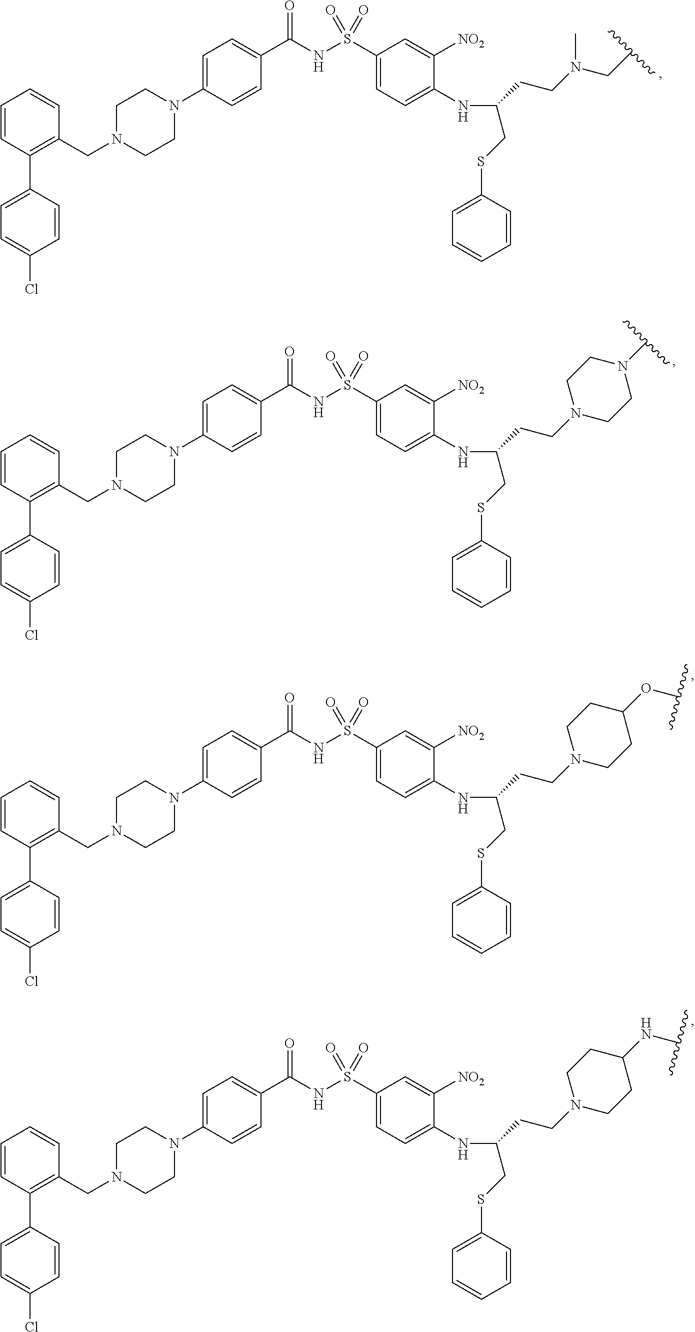 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

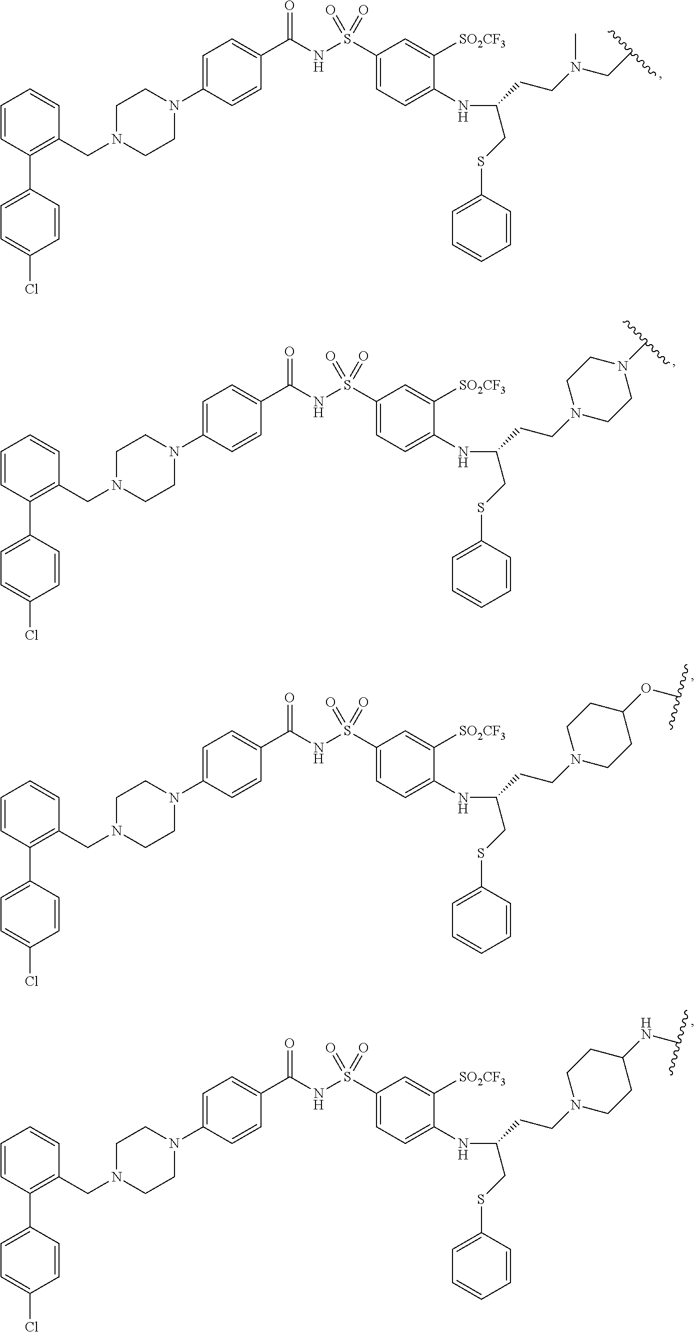

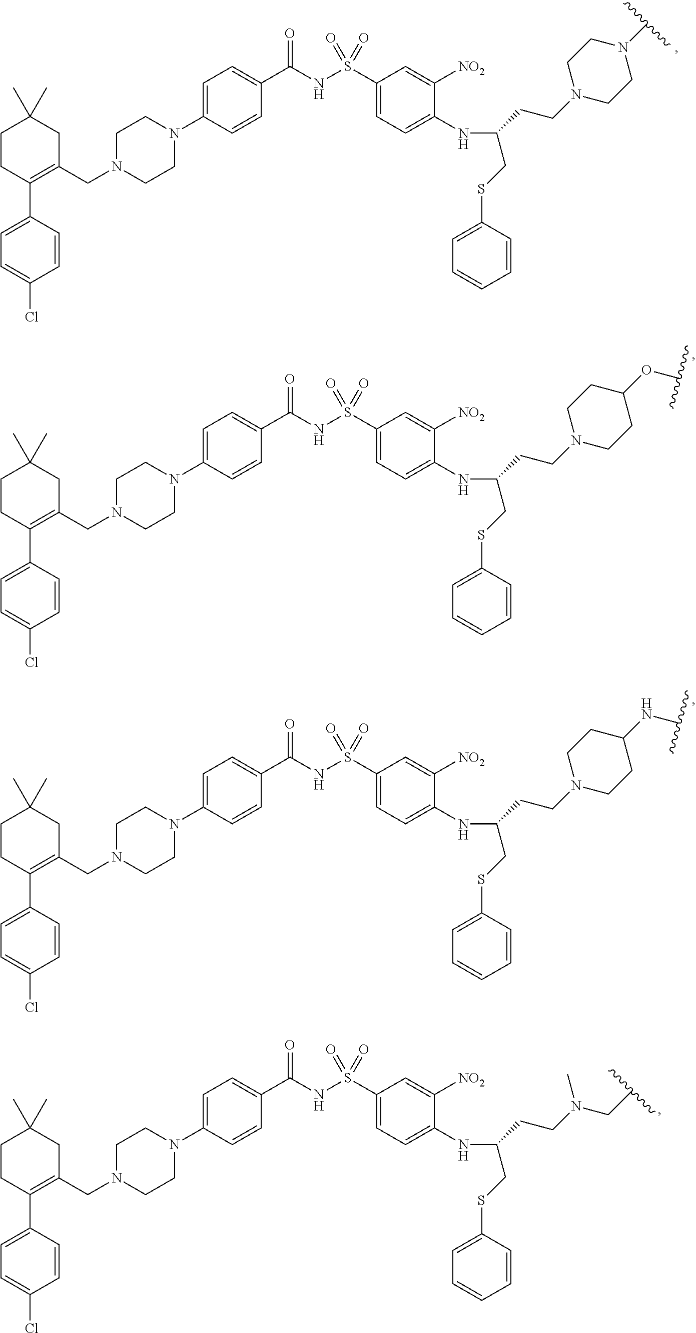 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

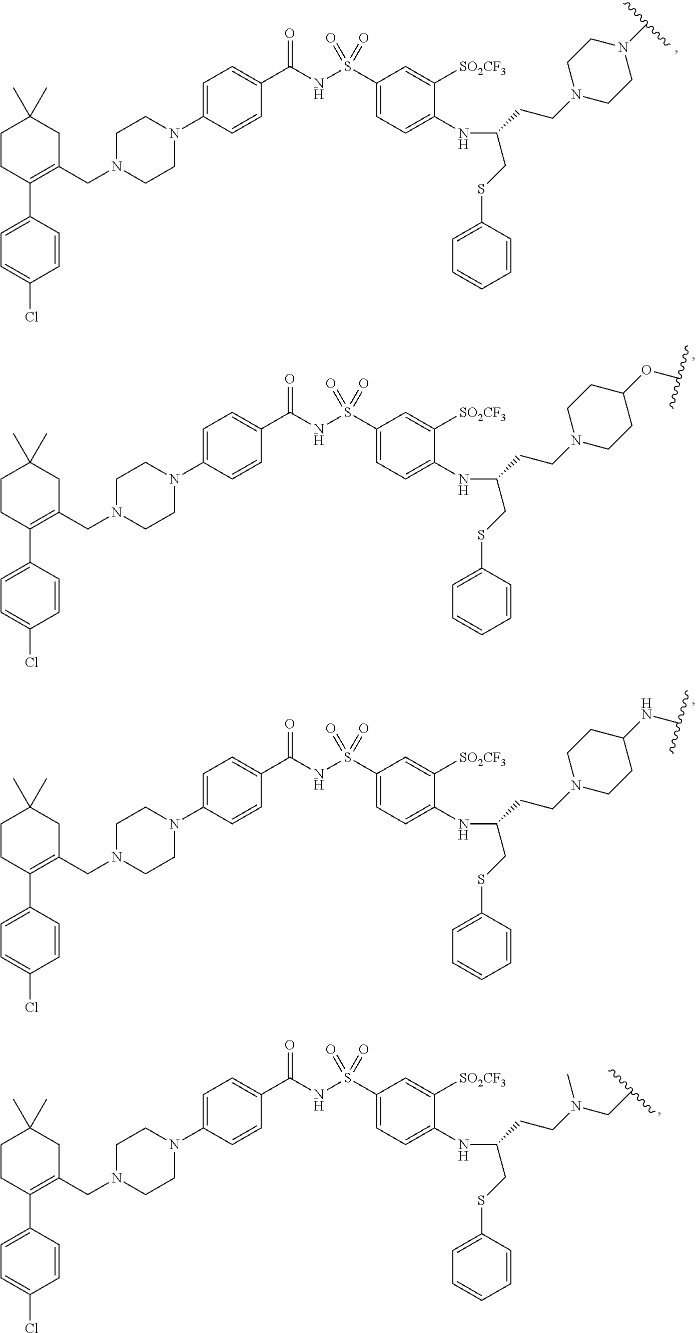

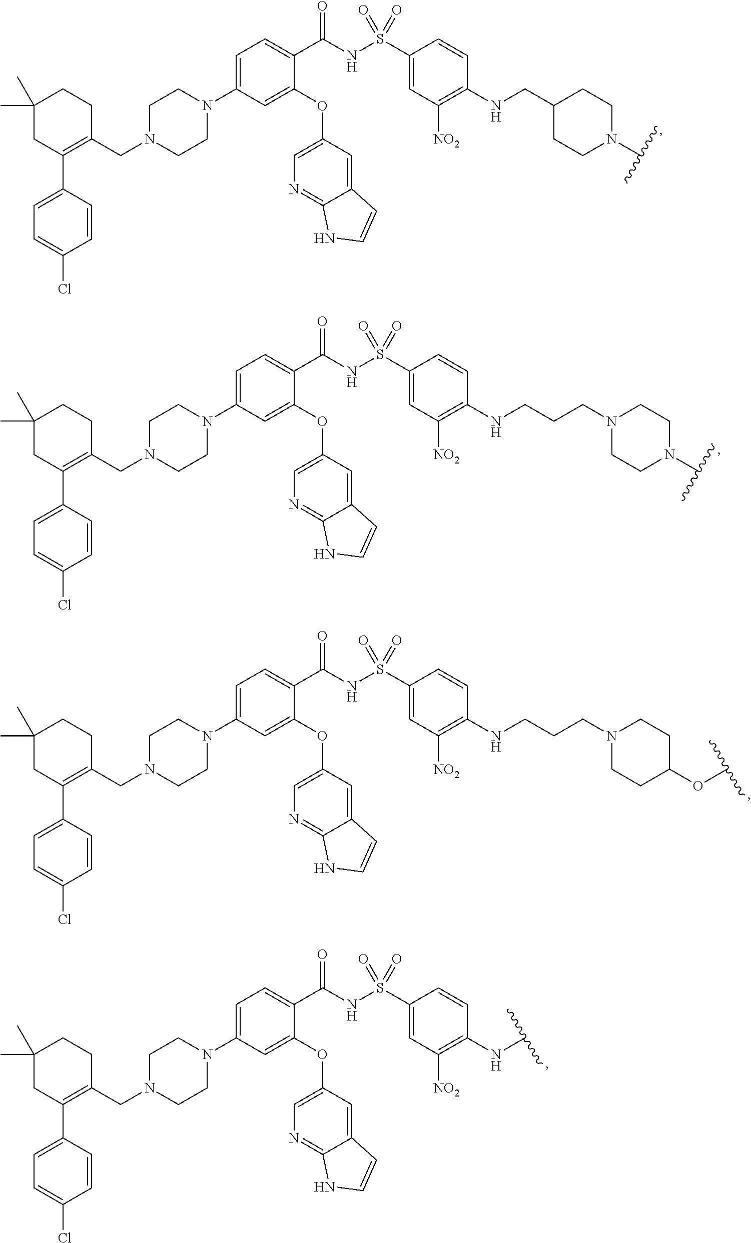 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *OC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

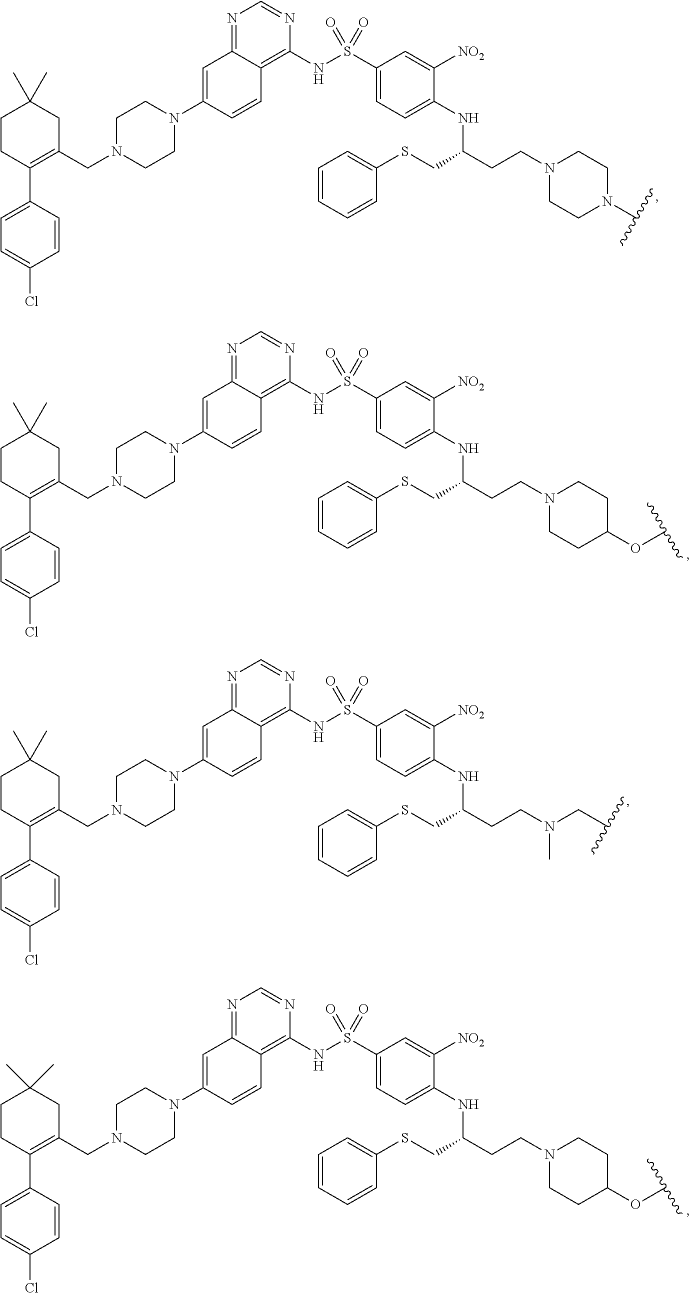 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

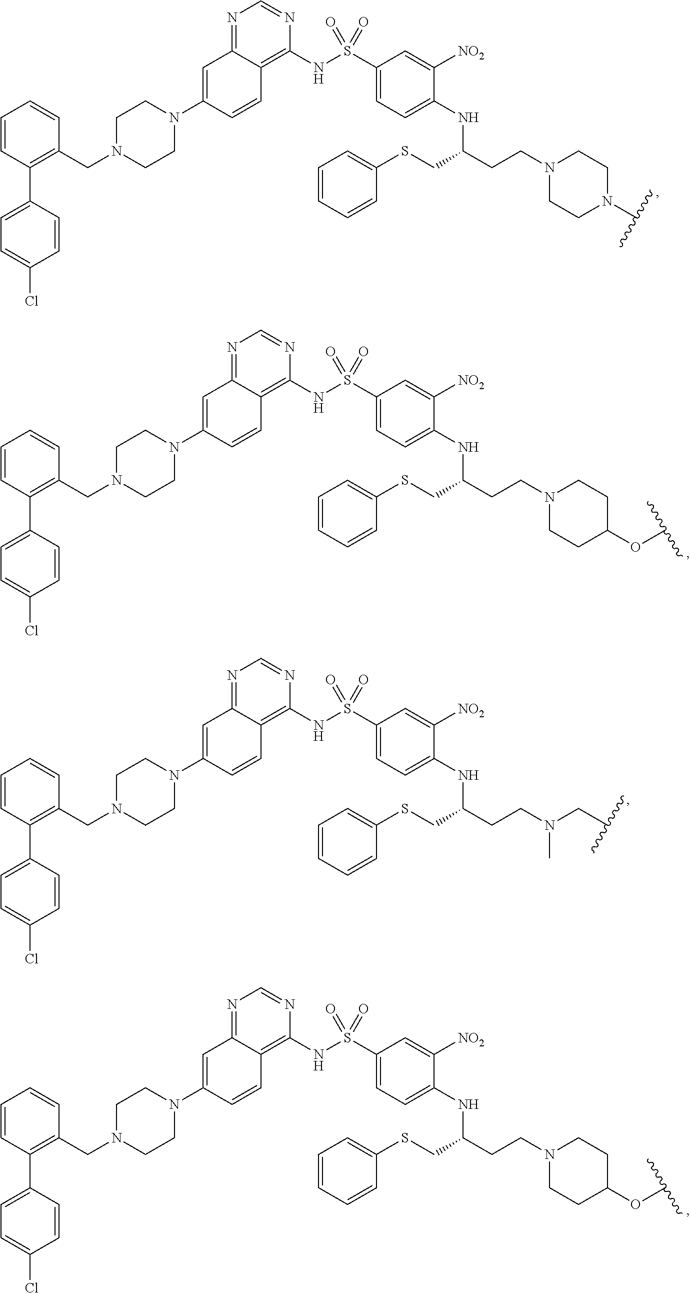 [SMILES: *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *CN(C)CC[C@H](CSc1ccccc1)Nc1ccc(S(=O)(=O)Nc2ncnc3cc(N4CCN(Cc5ccccc5-c5ccc(Cl)cc5)CC4)ccc23)cc1[N+](=O)[O-] ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

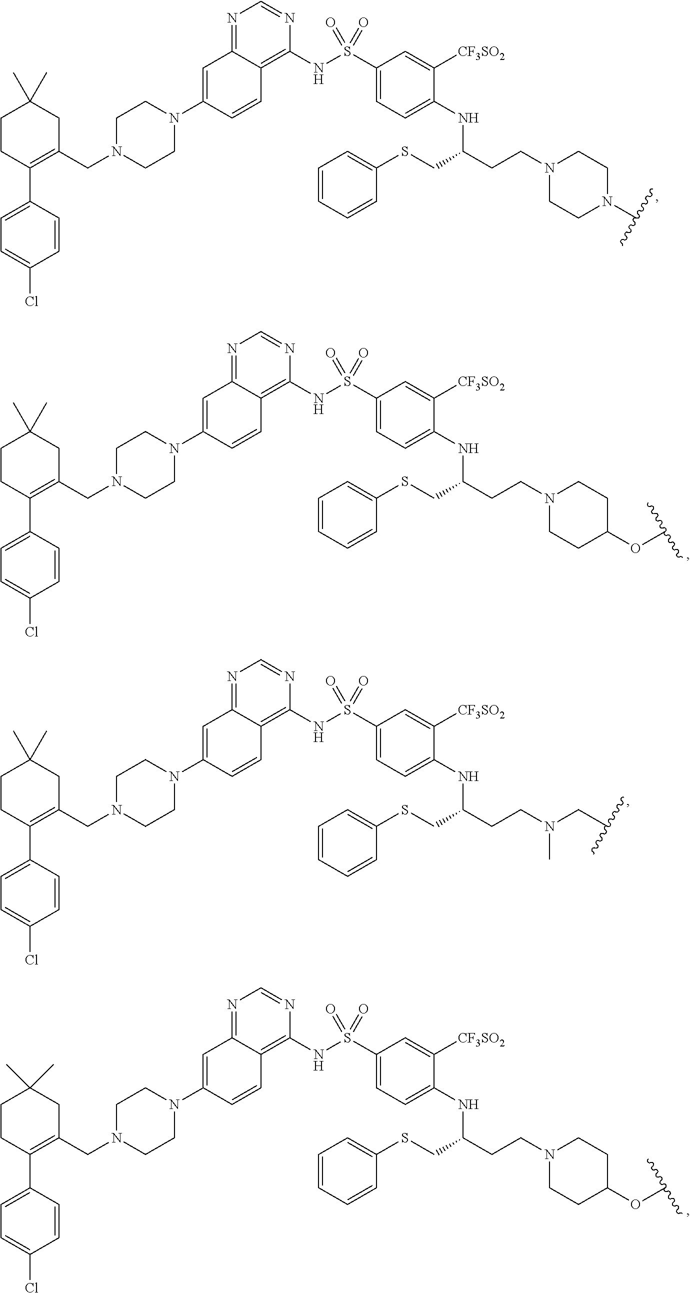

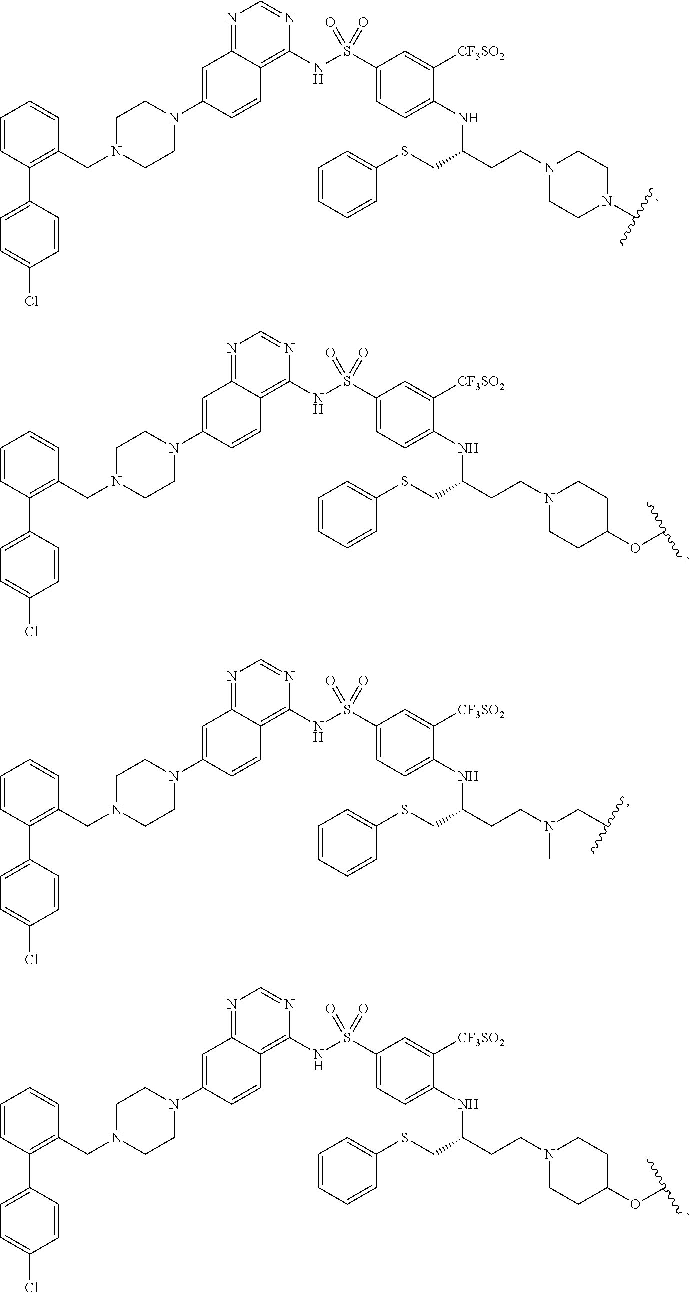

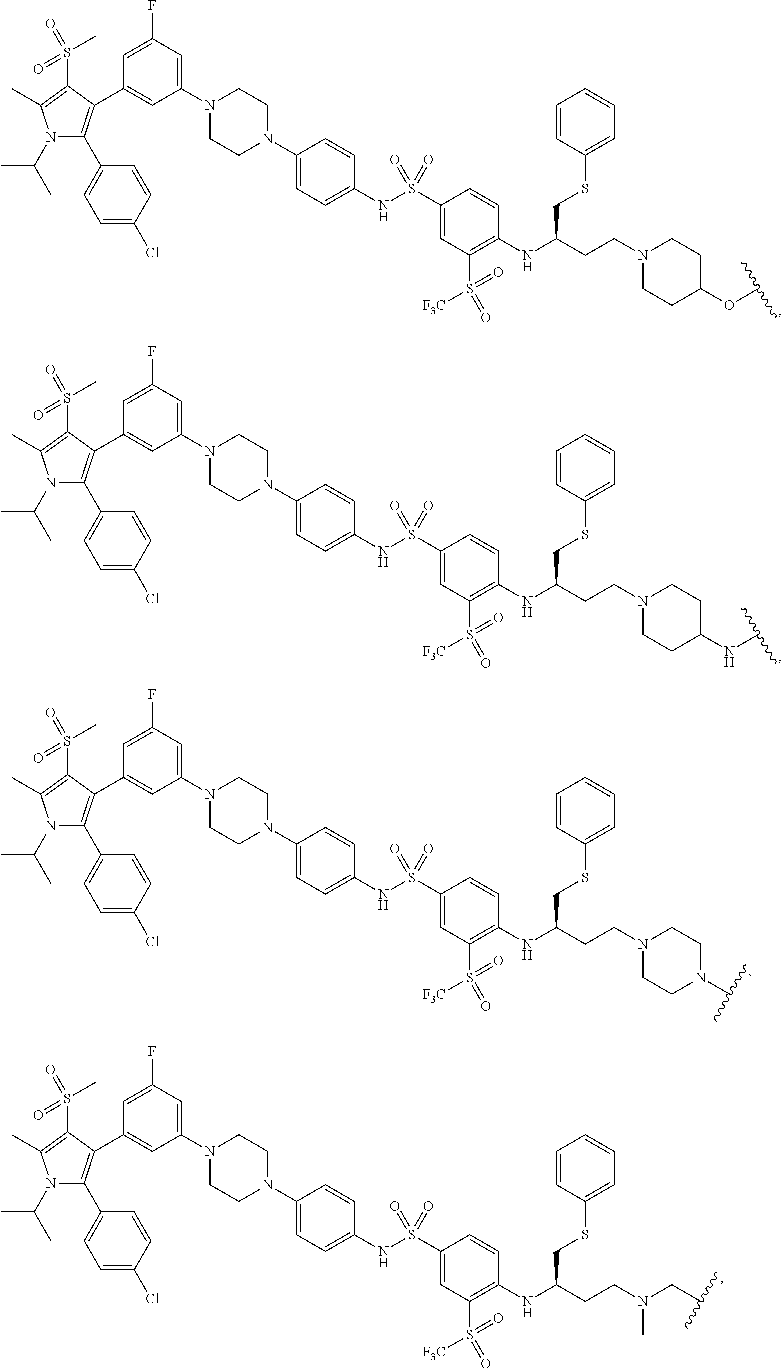

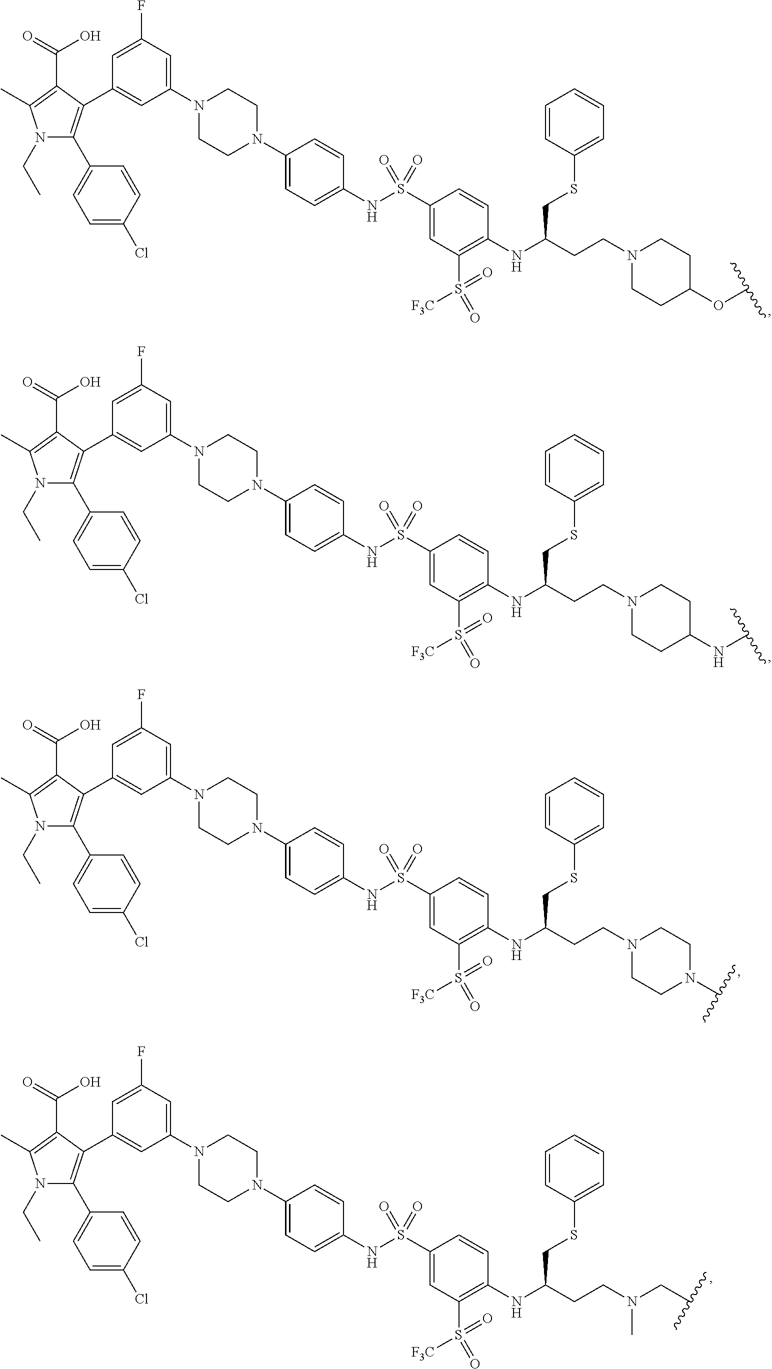

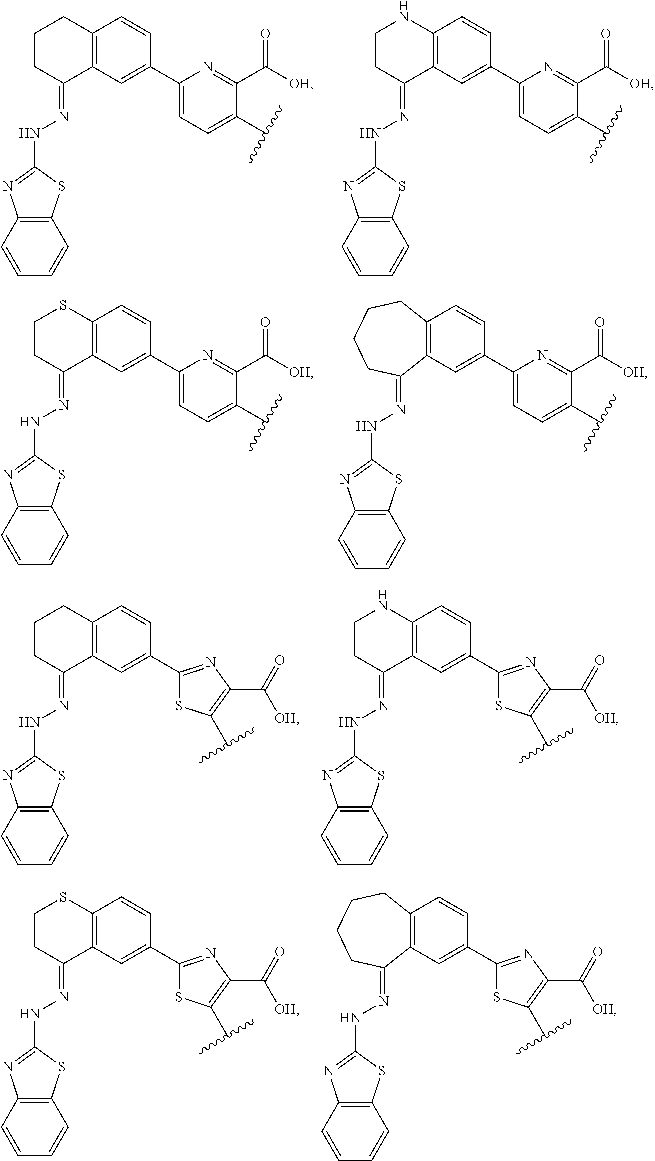

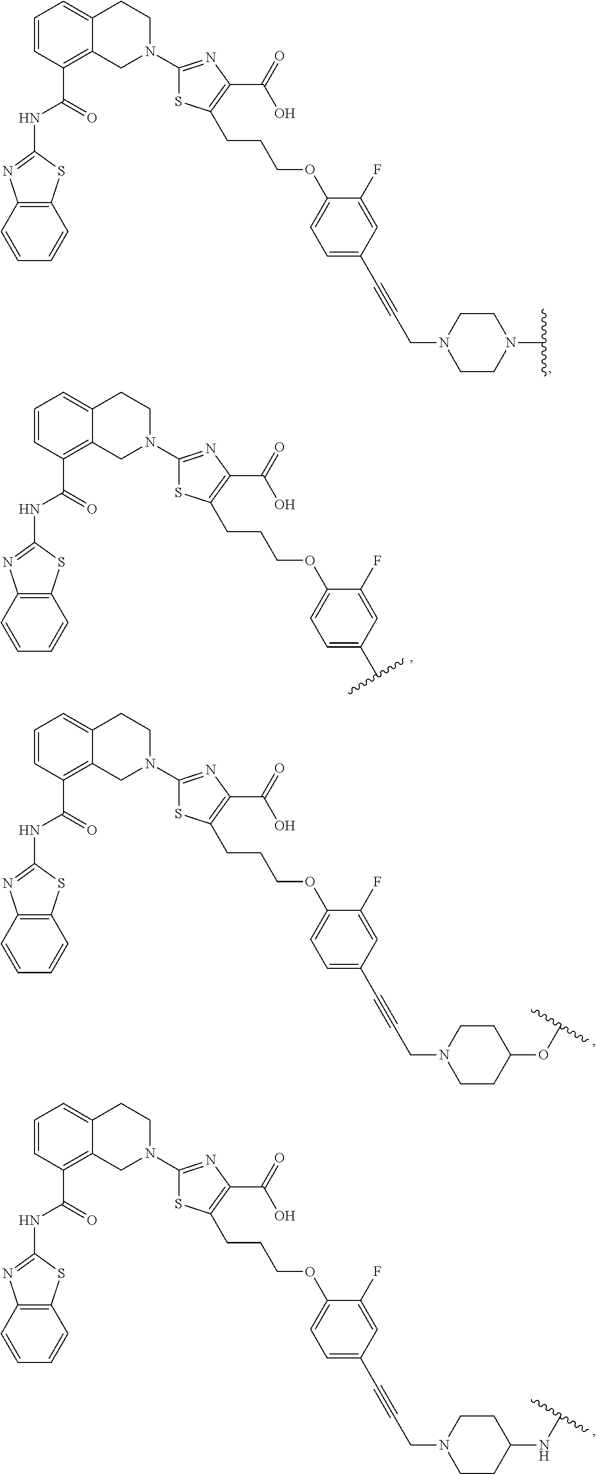 [SMILES: *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1 ; *NC1CCN(CC#Cc2ccc(OCCCc3sc(N4CCc5cccc(C(=O)Nc6nc7ccccc7s6)c5C4)nc3C(=O)O)c(F)c2)CC1]

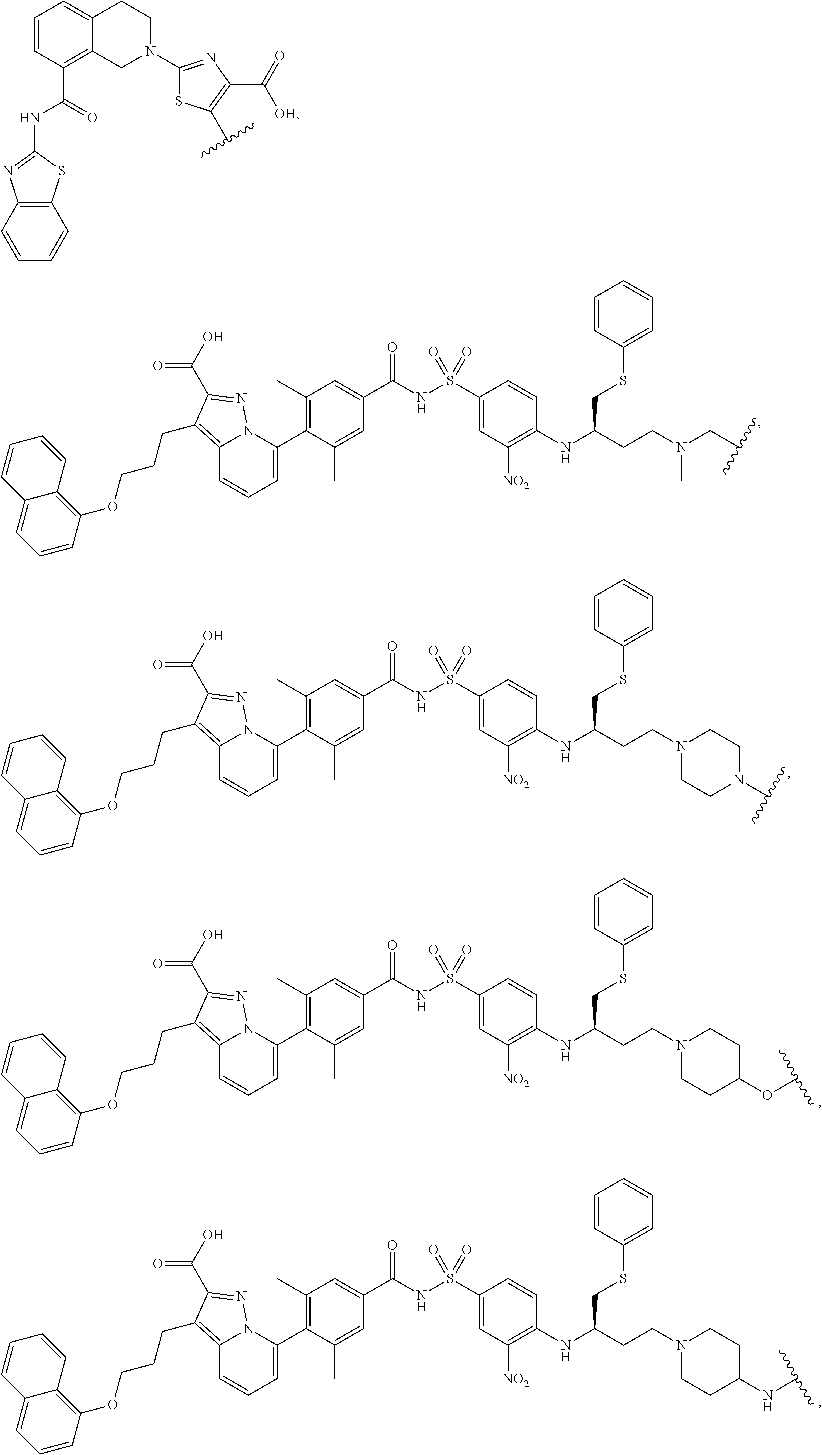

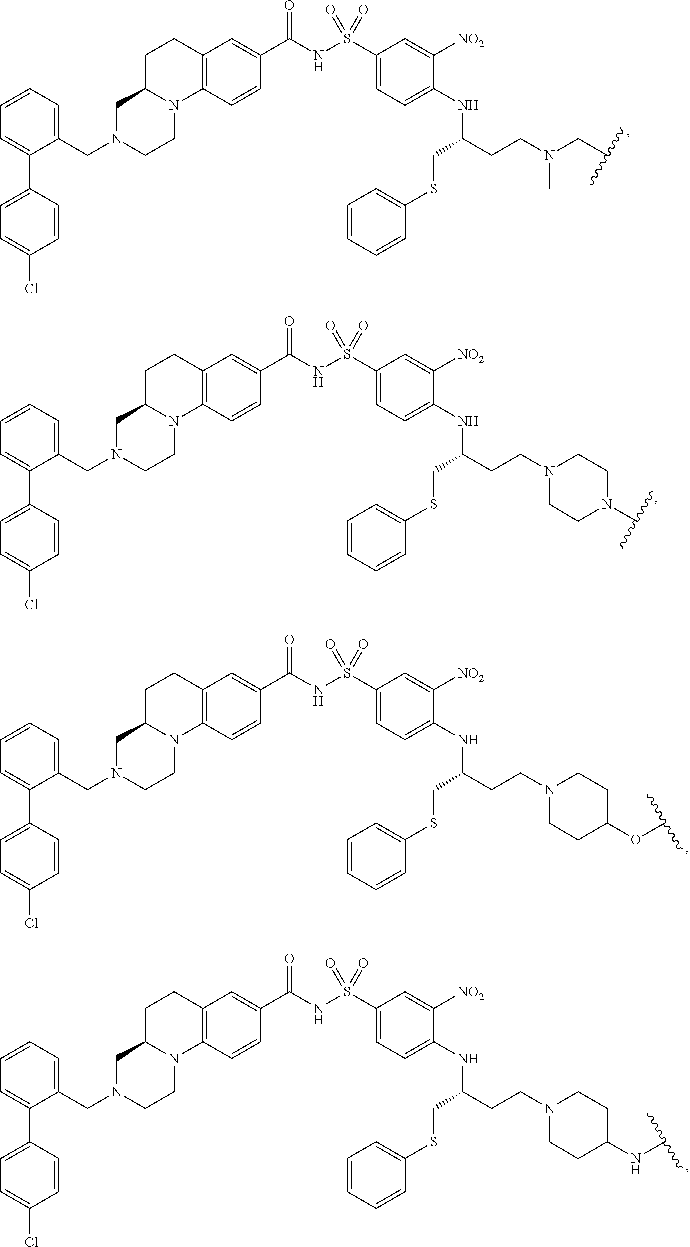 [SMILES: *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1]

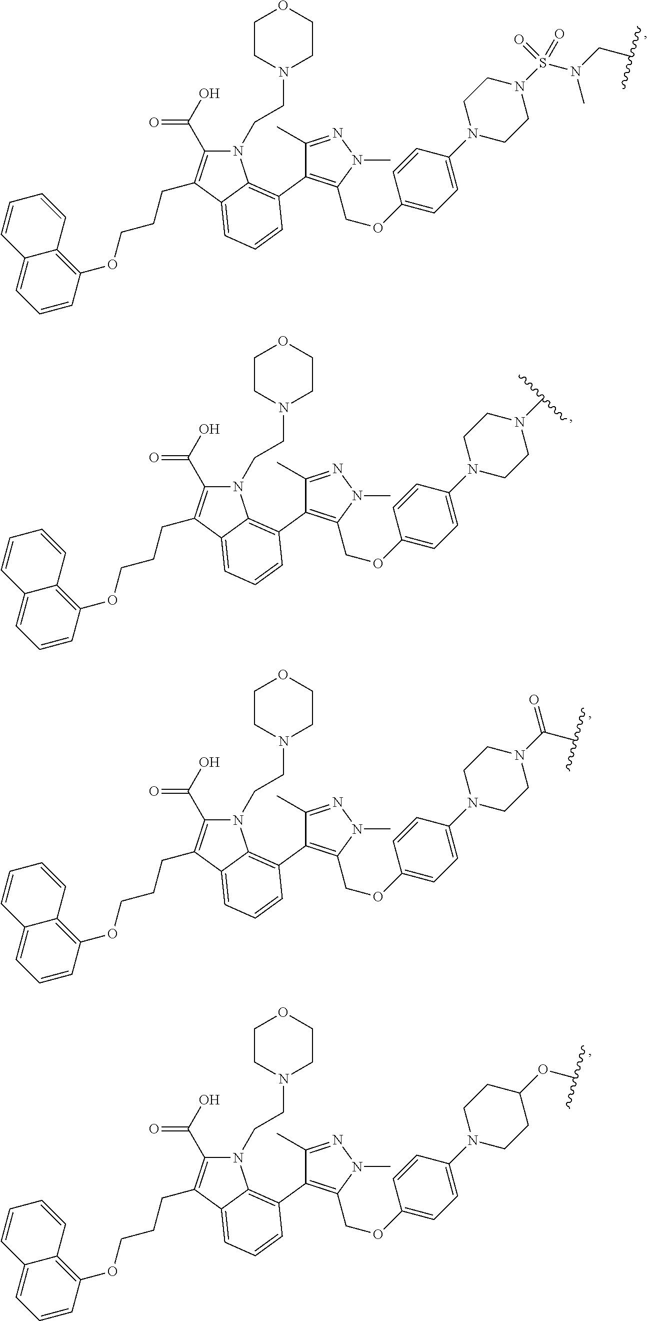 [SMILES: *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *OC1CCN(c2ccc(OCc3c(-c4cccc5c(CCCOc6cccc7ccccc67)c(C(=O)O)n(CCN6CCOCC6)c45)c(C)nn3C)cc2)CC1]

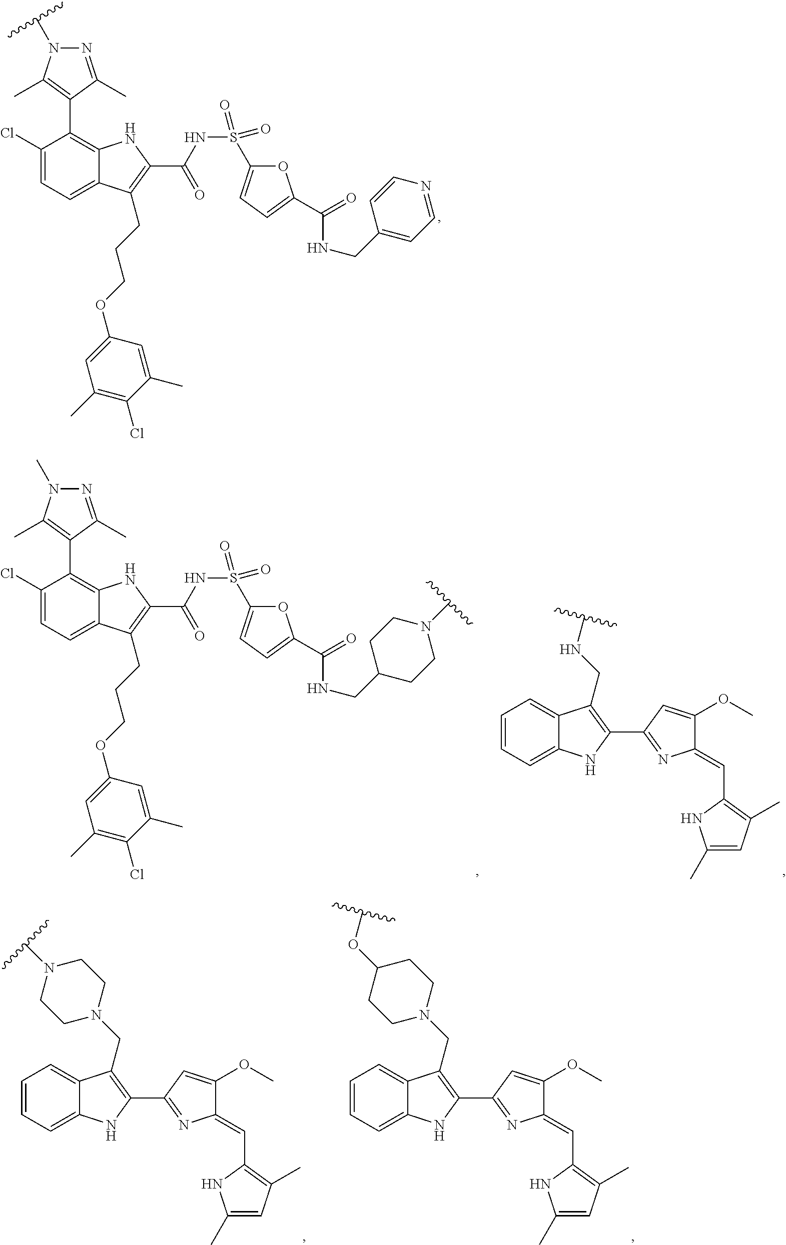 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12 ; *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1]

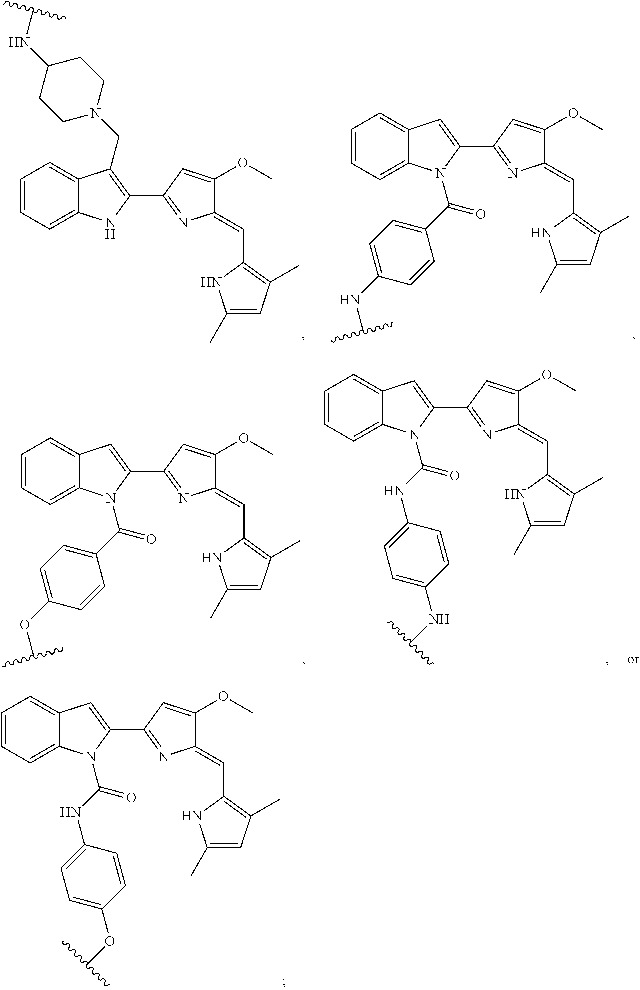

R³ may be absent, an unsubstituted C₁-C₆ alkyl, or a substituted or unsubstituted C₃-C₆ ketone; A may be absent, a bond, or a triazole; n may be 0 to 3; R⁴ may be a bond or a substituted or unsubstituted C₁-C₁₀ alkyl; and wherein R² may be

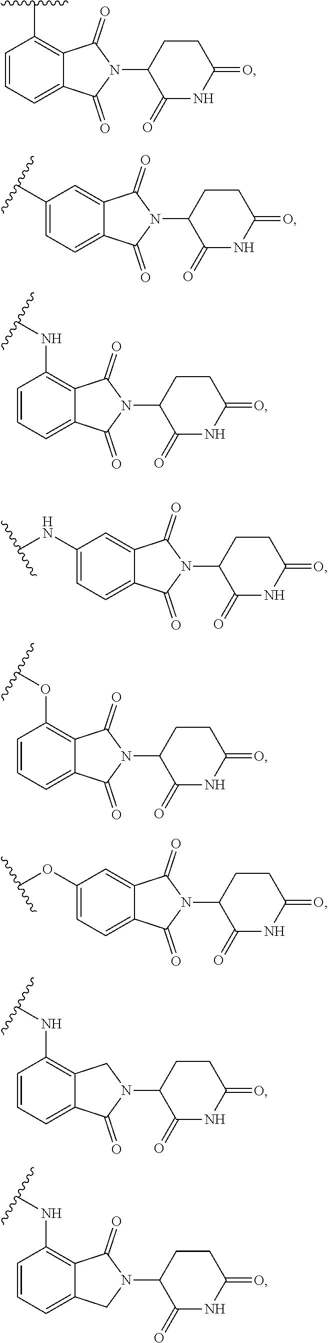

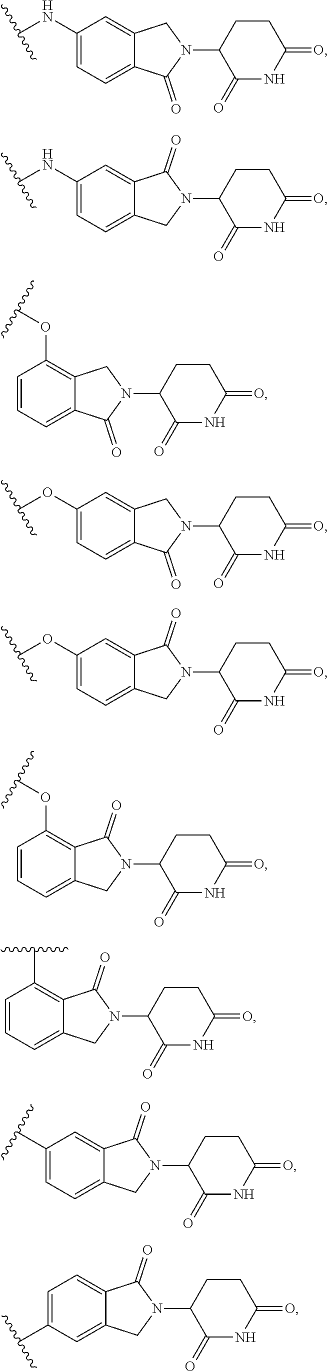

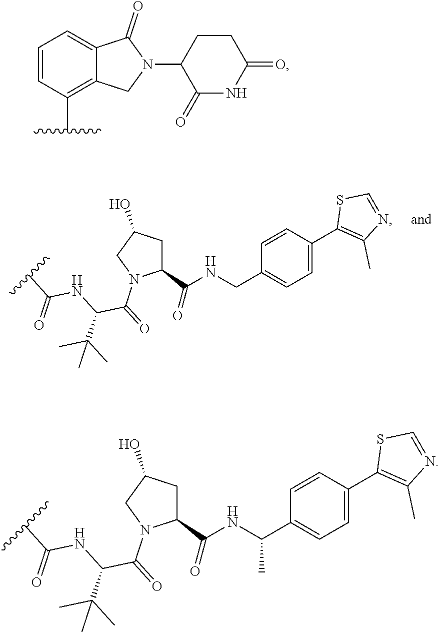 [SMILES: *c1cccc2c1CN(C1CCC(=O)NC1=O)C2=O ; *C(=O)N[C@H](C(=O)N1C[C@H](O)C[C@H]1C(=O)NCc1ccc(-c2scnc2C)cc1)C(C)(C)C ; "and" ; *C(=O)N[C@H](C(=O)N1C[C@H](O)C[C@H]1C(=O)N[C@@H](C)c1ccc(-c2scnc2C)cc1)C(C)(C)C]

In still another embodiment, a compound of the disclosure comprises Formula (II), wherein R¹ may be

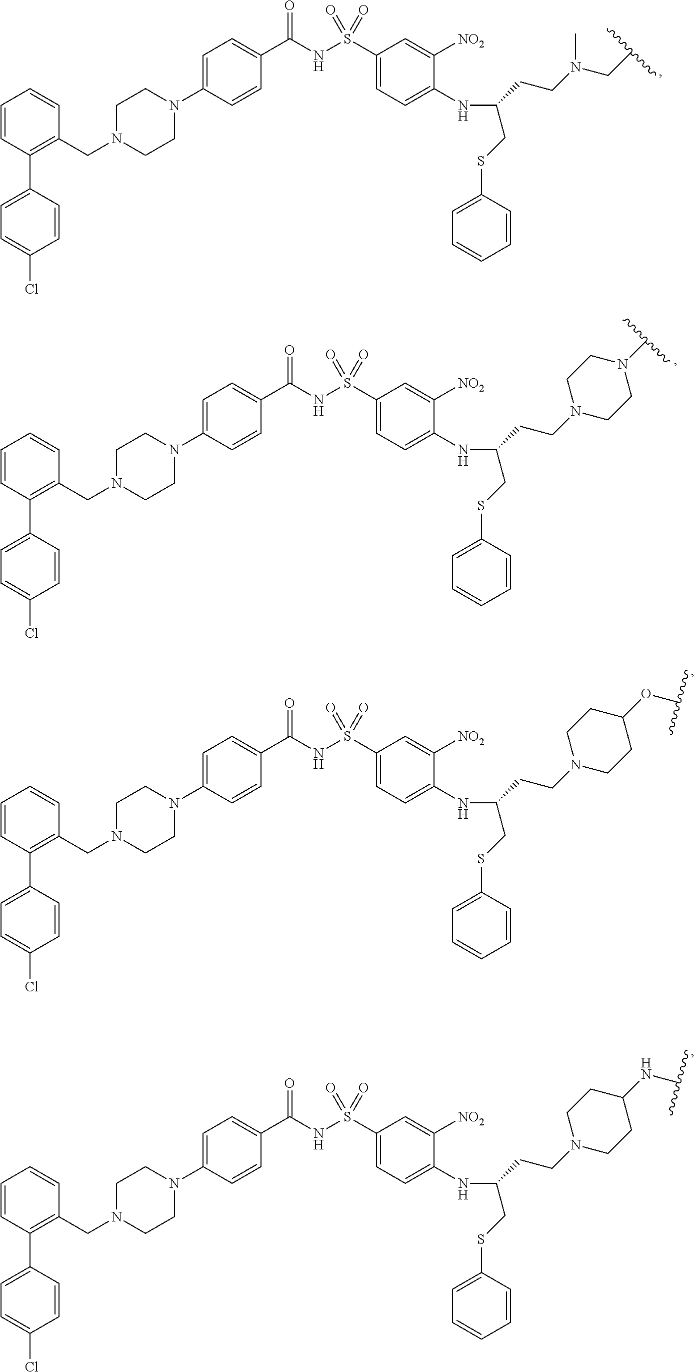 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

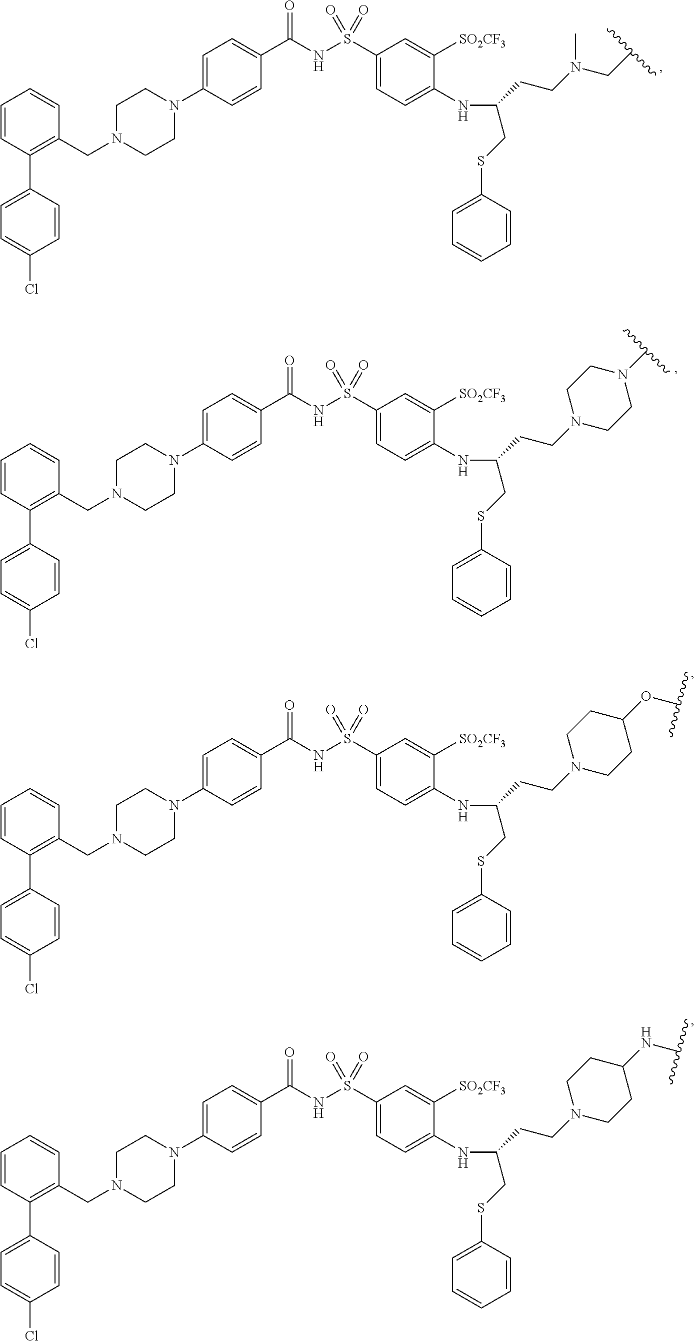

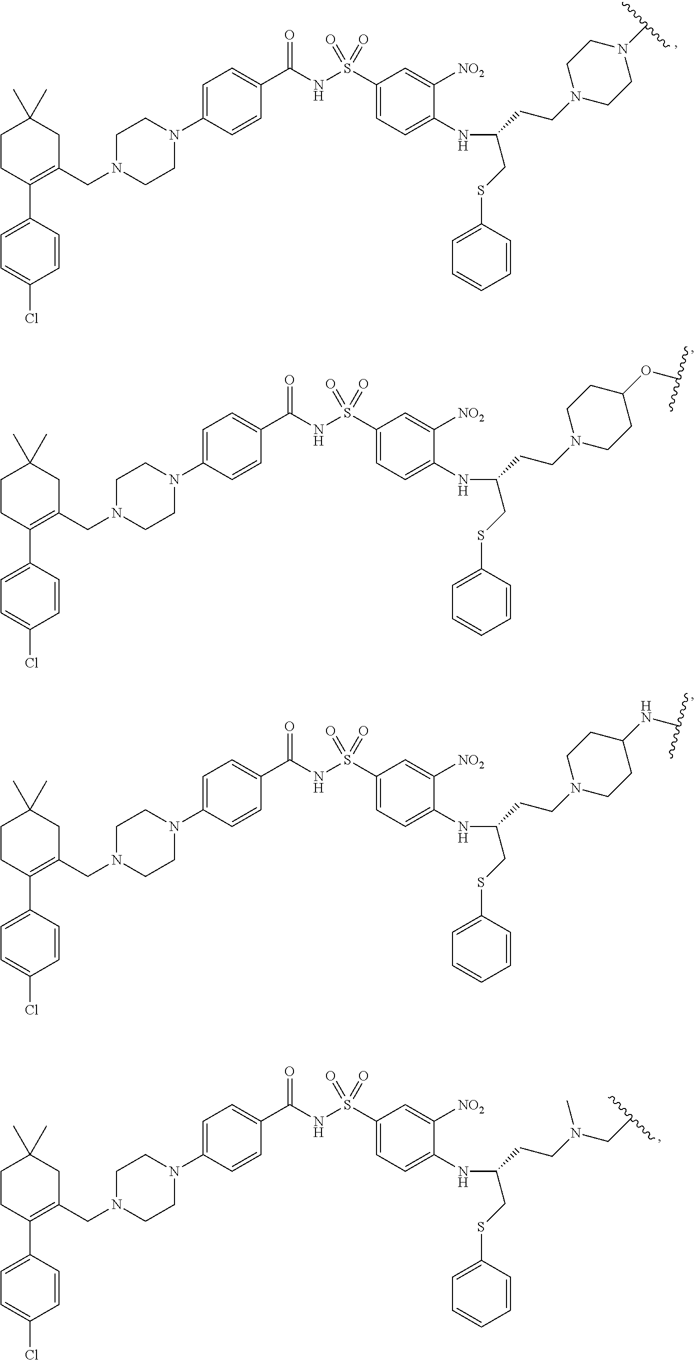 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

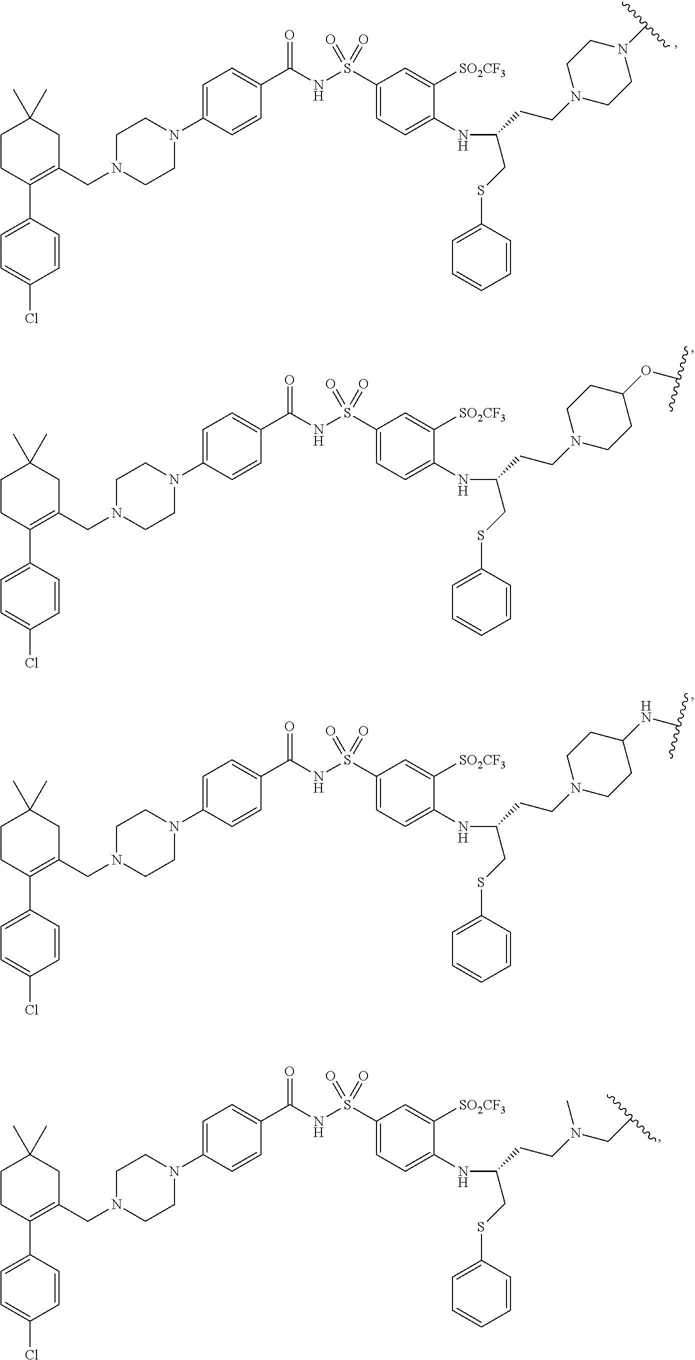

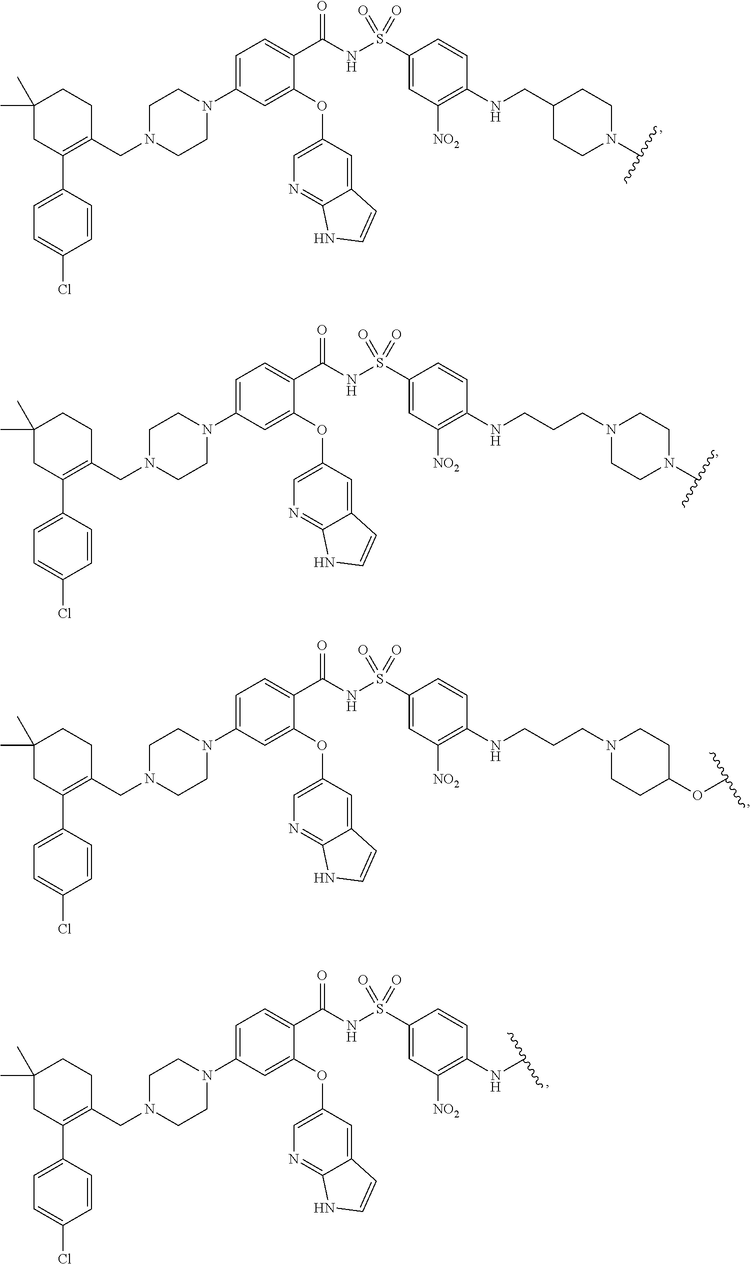 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *OC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

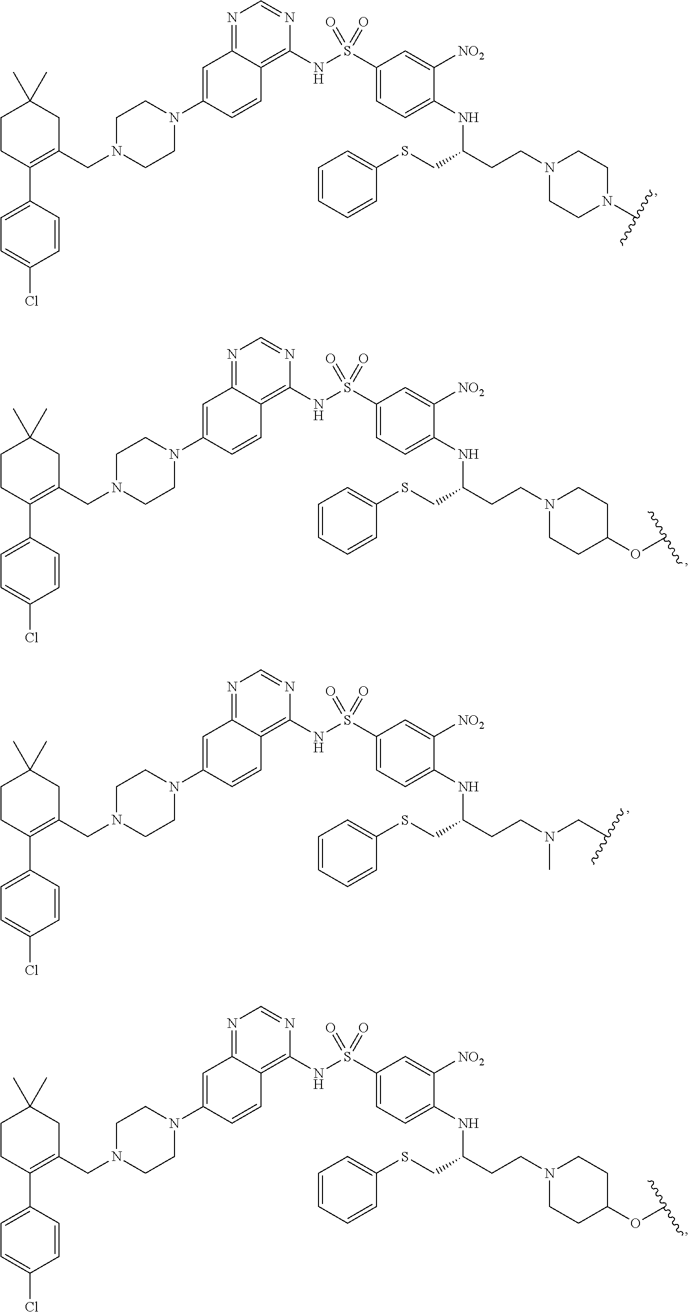 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

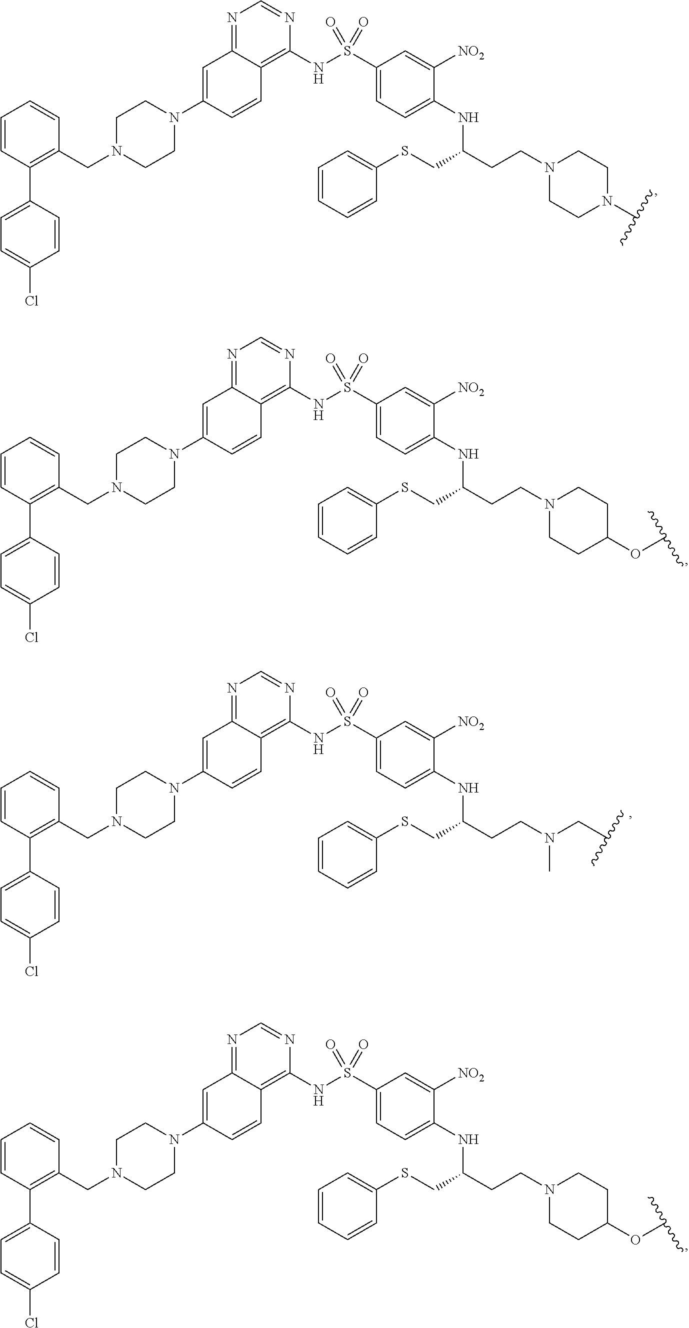 [SMILES: *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *CN(C)CC[C@H](CSc1ccccc1)Nc1ccc(S(=O)(=O)Nc2ncnc3cc(N4CCN(Cc5ccccc5-c5ccc(Cl)cc5)CC4)ccc23)cc1[N+](=O)[O-] ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

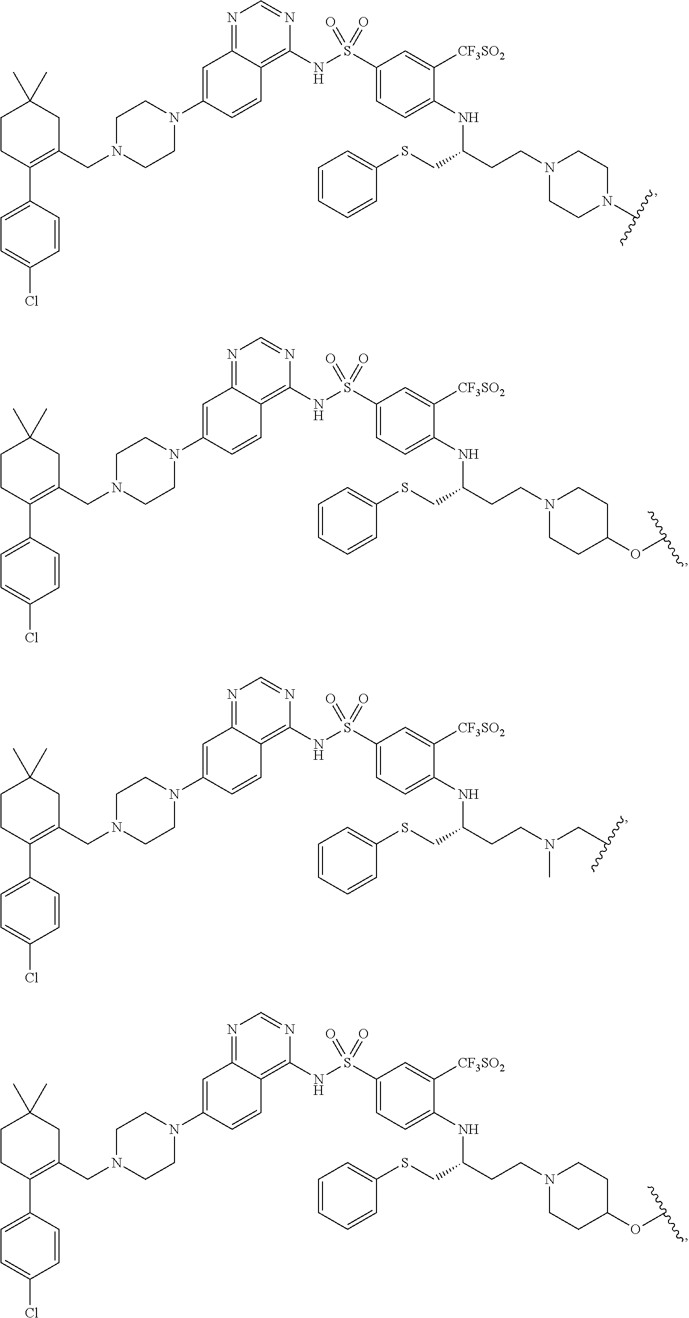

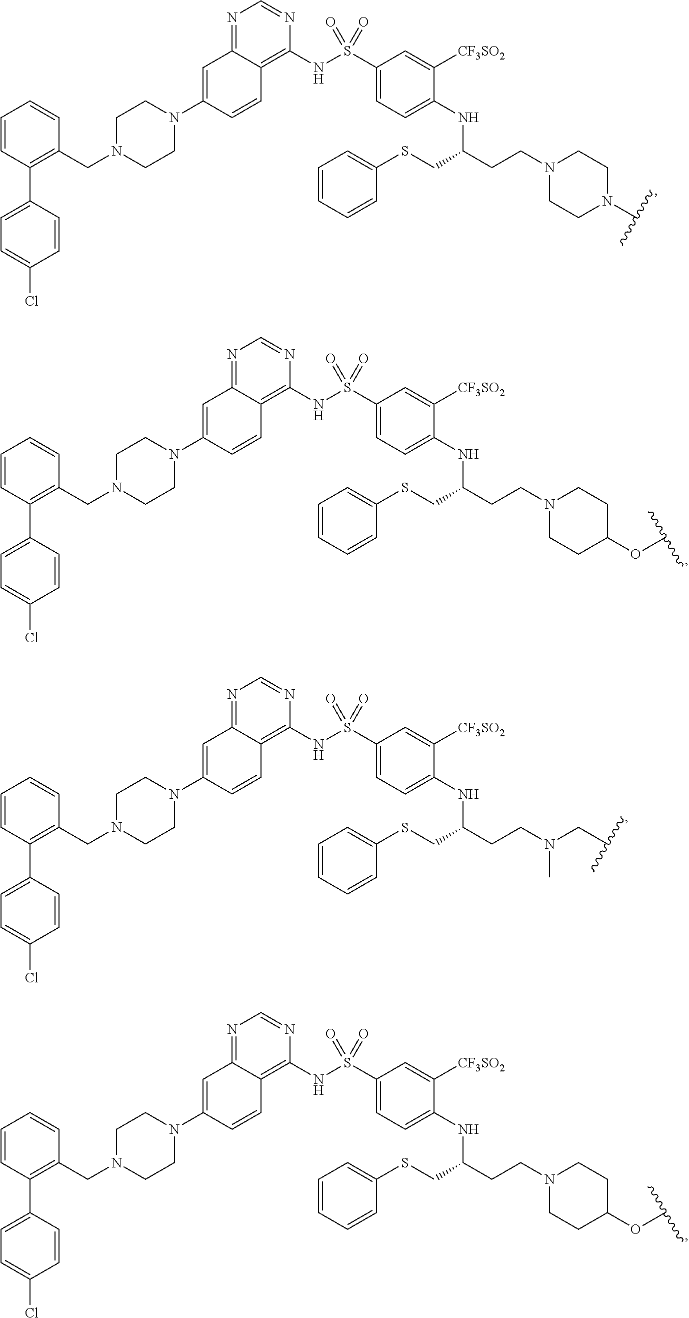

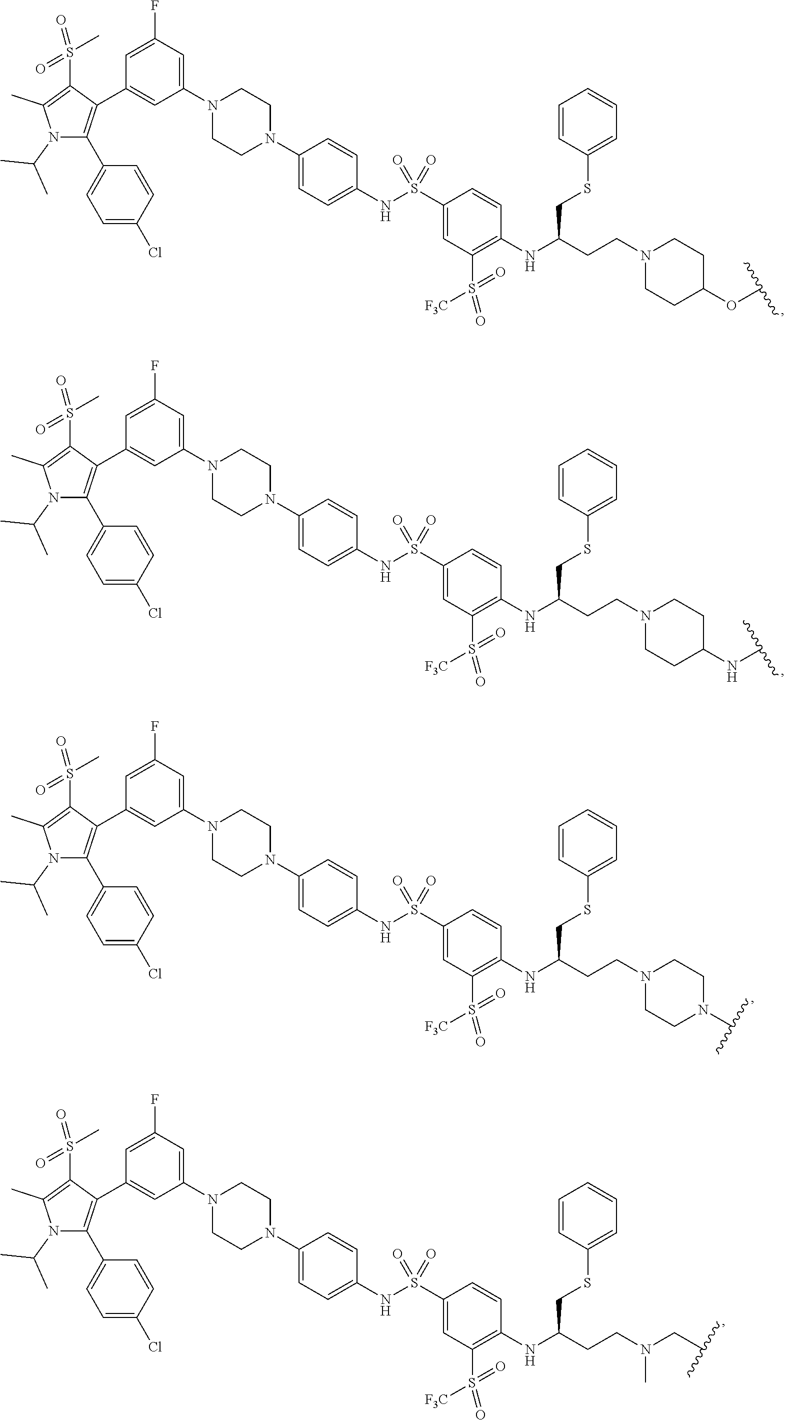

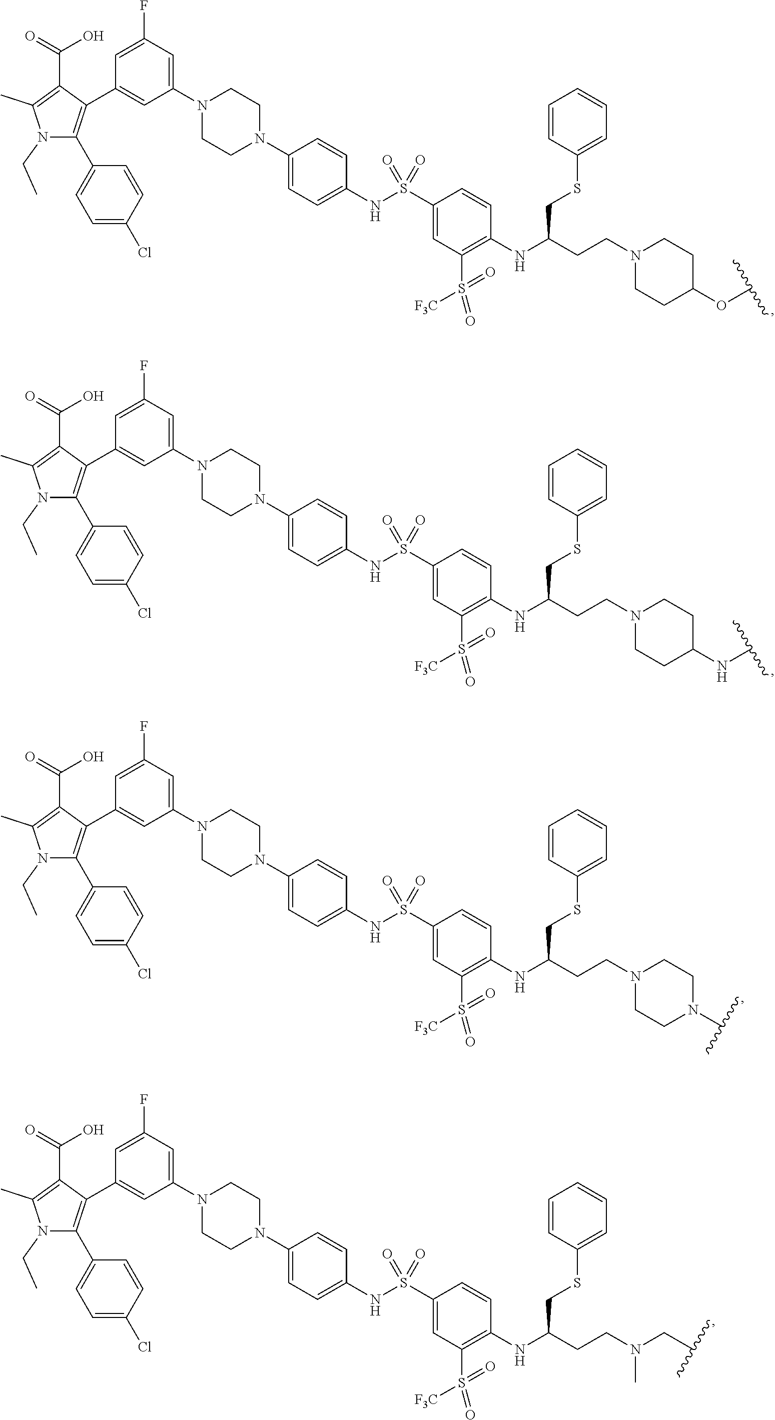

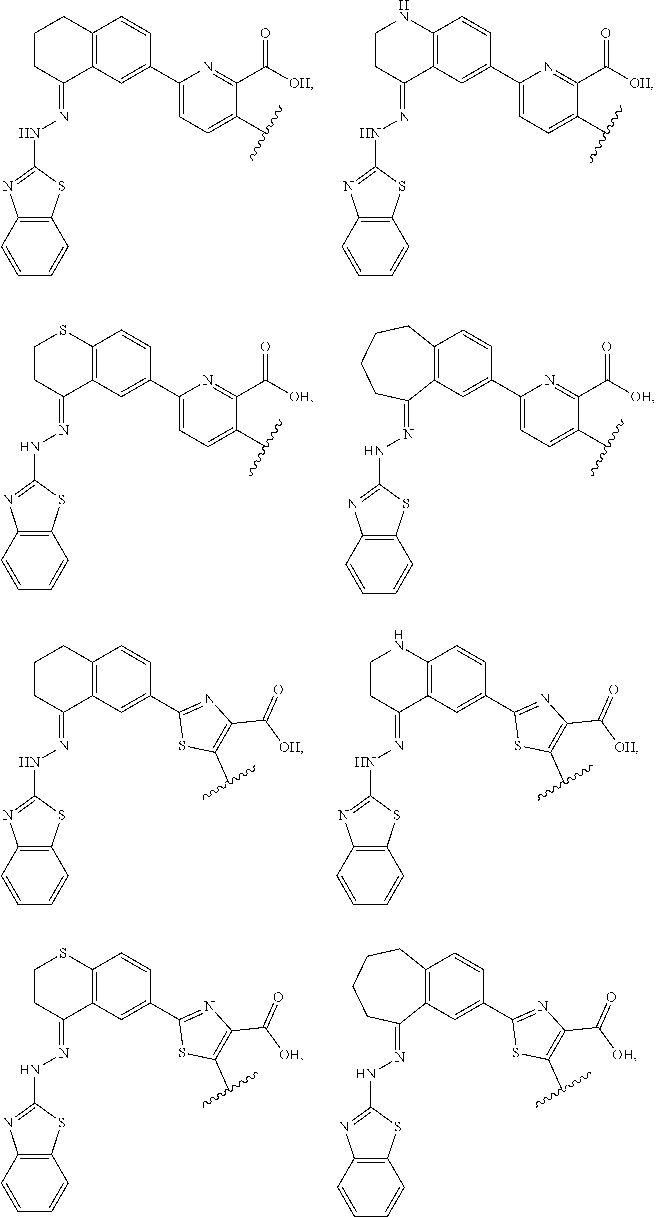

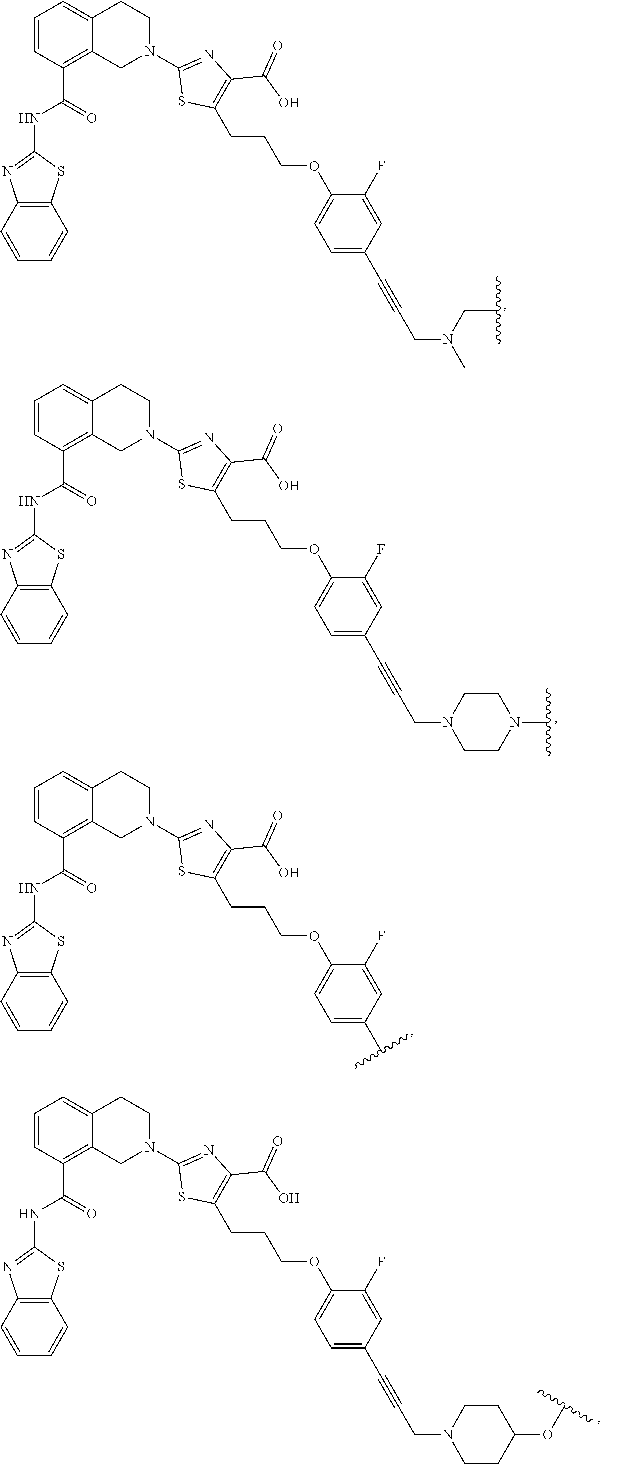 [SMILES: *CN(C)CC#Cc1ccc(OCCCc2sc(N3CCc4cccc(C(=O)Nc5nc6ccccc6s5)c4C3)nc2C(=O)O)c(F)c1 ; *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1]

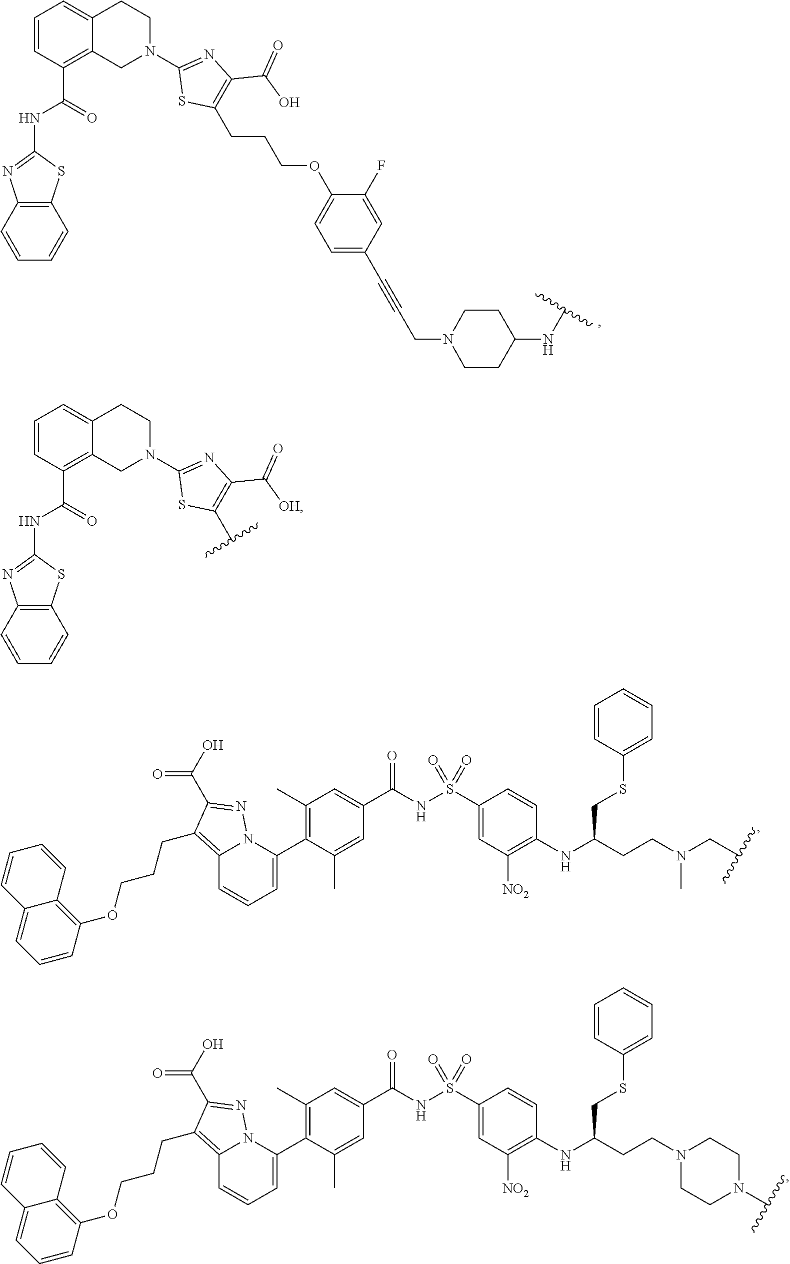 [SMILES: *NC1CCN(CC#Cc2ccc(OCCCc3sc(N4CCc5cccc(C(=O)Nc6nc7ccccc7s6)c5C4)nc3C(=O)O)c(F)c2)CC1 ; *c1sc(N2CCc3cccc(C(=O)Nc4nc5ccccc5s4)c3C2)nc1C(=O)O ; *CN(C)CC[C@H](CSc1ccccc1)Nc1ccc(S(=O)(=O)NC(=O)c2cc(C)c(-c3cccc4c(CCCOc5cccc6ccccc56)c(C(=O)O)nn34)c(C)c2)cc1[N+](=O)[O-] ; *N1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1]

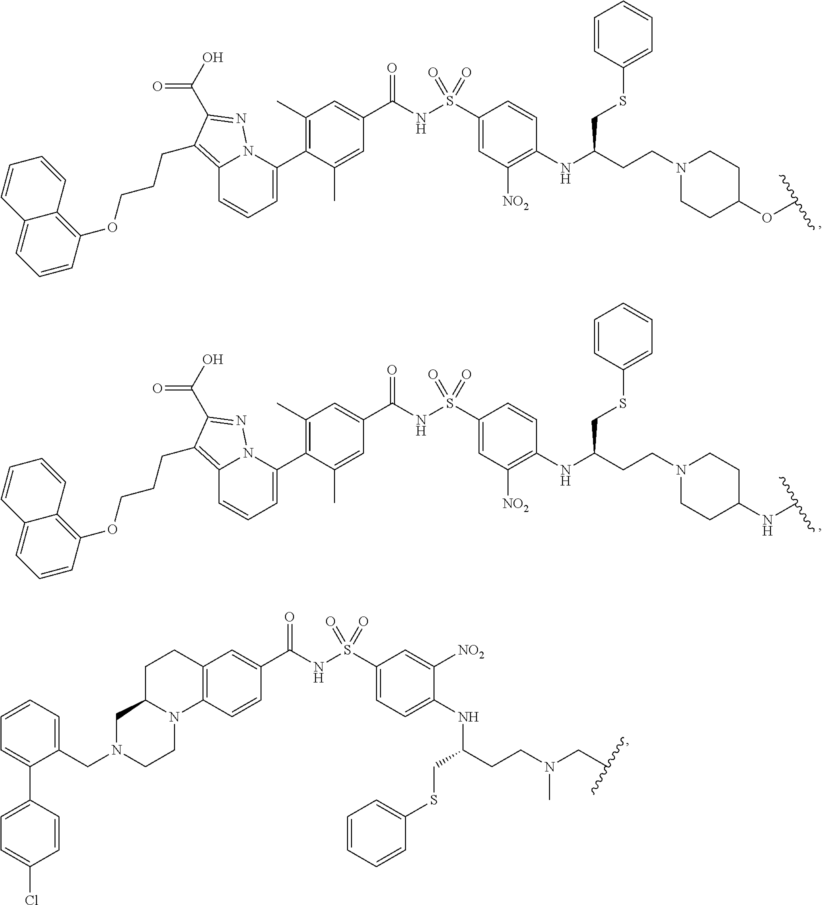 [SMILES: *OC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *NC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-]]

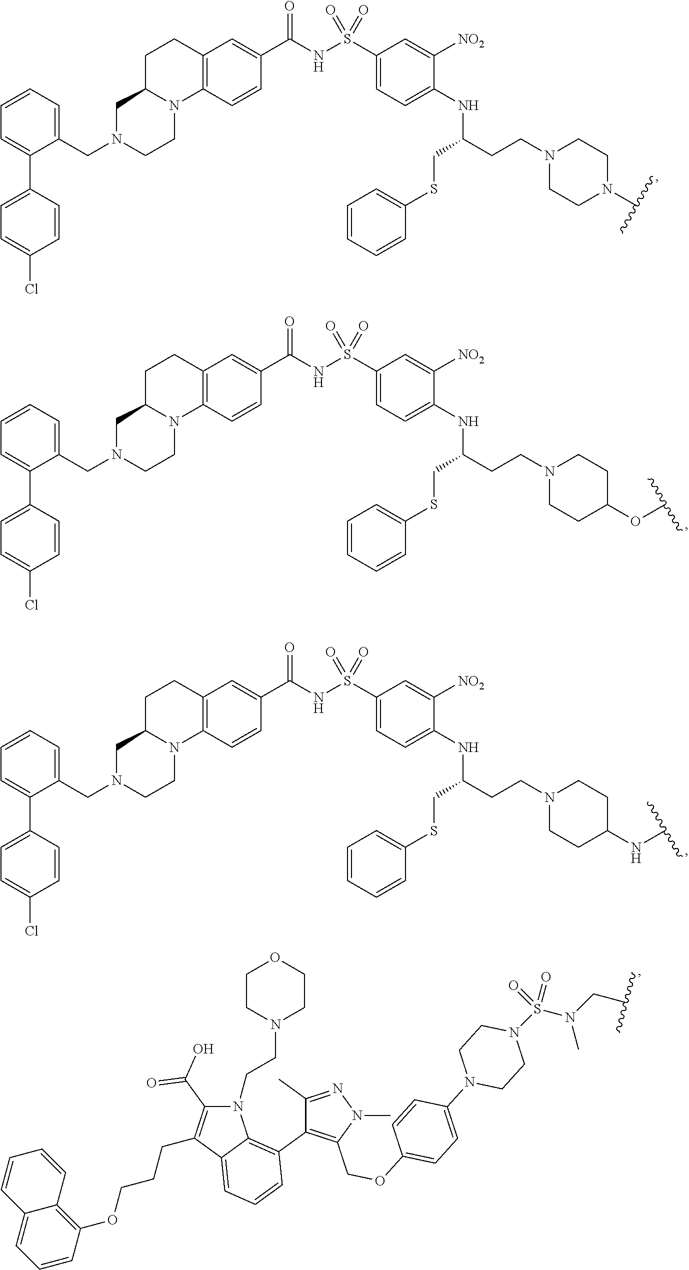 [SMILES: *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1]

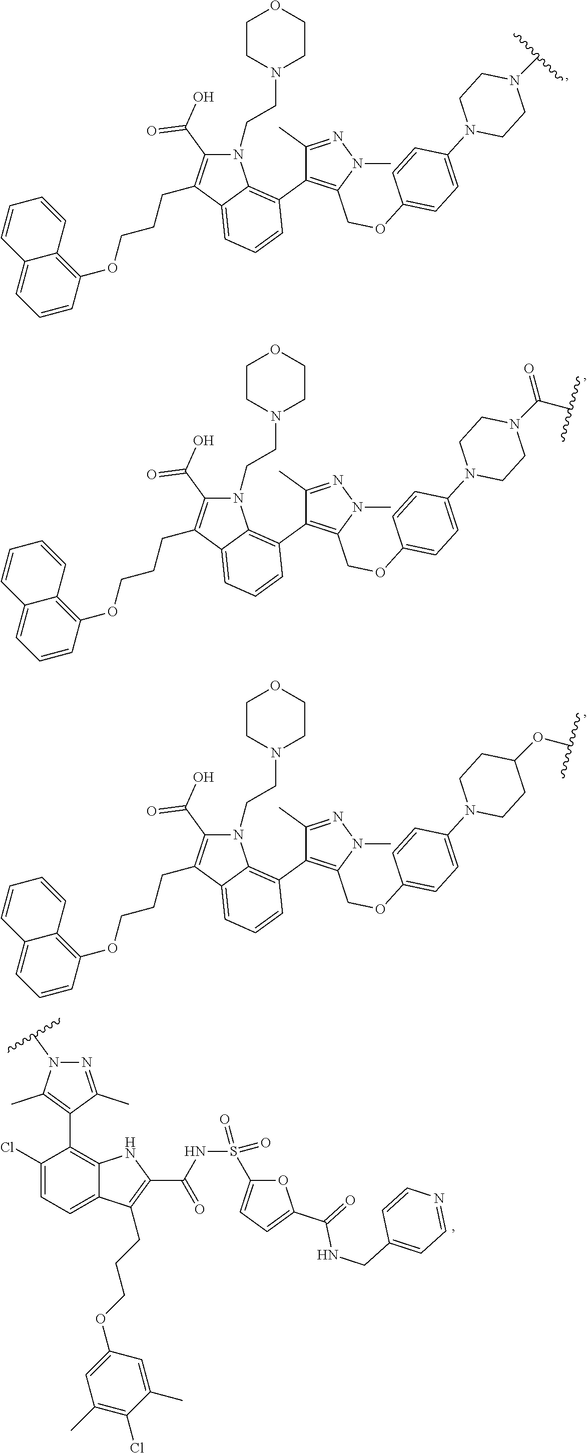

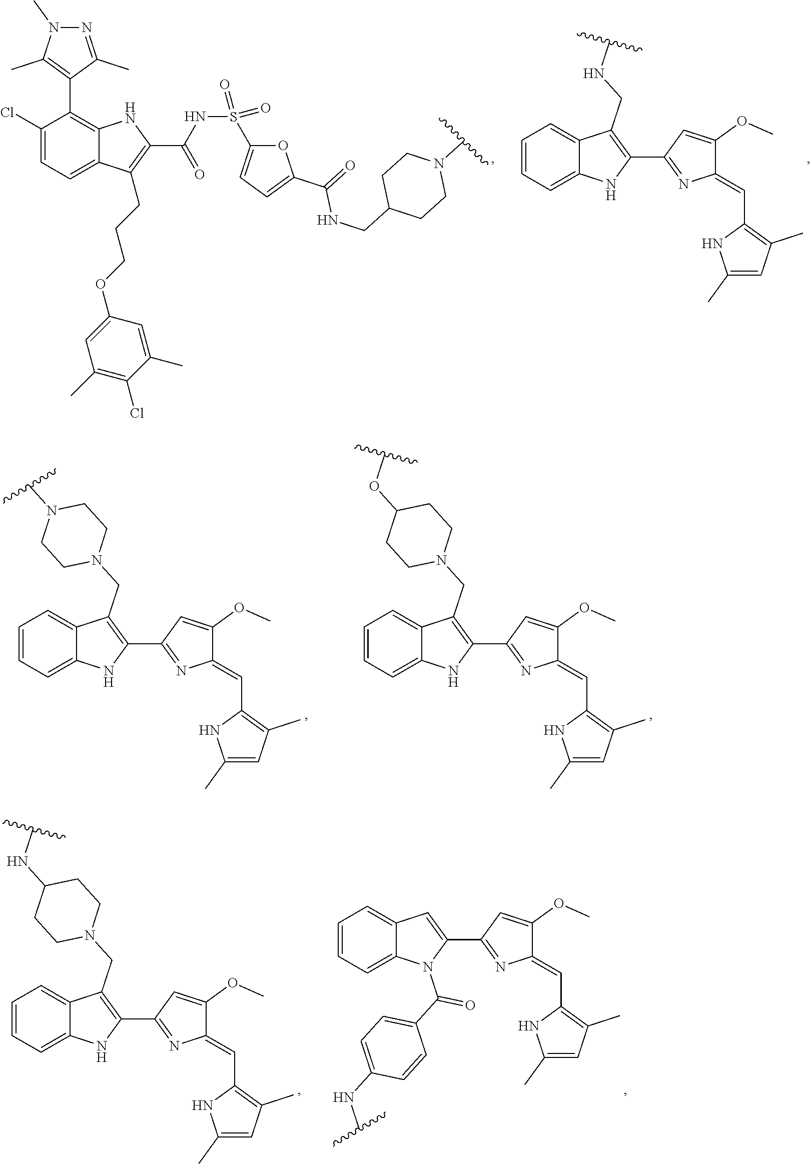 [SMILES: *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12 ; *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1 ; *NC1CCN(Cc2c(C3=N/C(=C\c4[nH]c(C)cc4C)C(OC)=C3)[nH]c3ccccc23)CC1 ; *Nc1ccc(C(=O)n2c(C3=N/C(=C\c4[nH]c(C)cc4C)C(OC)=C3)cc3ccccc32)cc1]

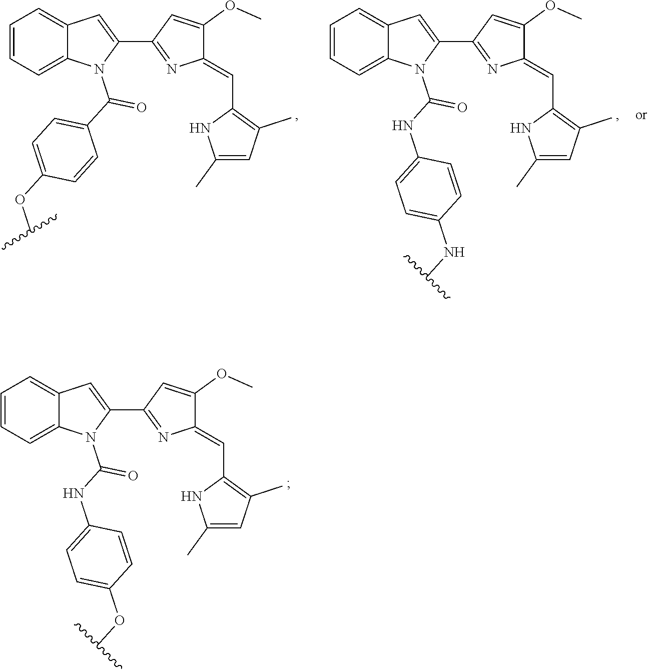 [SMILES: *Oc1ccc(C(=O)n2c(C3=N/C(=C\c4[nH]c(C)cc4C)C(OC)=C3)cc3ccccc32)cc1 ; *Nc1ccc(NC(=O)n2c(C3=N/C(=C\c4[nH]c(C)cc4C)C(OC)=C3)cc3ccccc32)cc1 ; *Oc1ccc(NC(=O)n2c(C3=N/C(=C\c4[nH]c(C)cc4C)C(OC)=C3)cc3ccccc32)cc1]

R³ may be absent, an unsubstituted C₁-C₆ alkyl, or a substituted or unsubstituted C₃-C₆ ketone; A may be absent, a bond, or a substituted or unsubstituted C₁-C₆ heterocyclic group; n may be 1 to 2; R⁴ may be a bond or a substituted or unsubstituted C₁-C₁₀ alkyl; and wherein R² may be

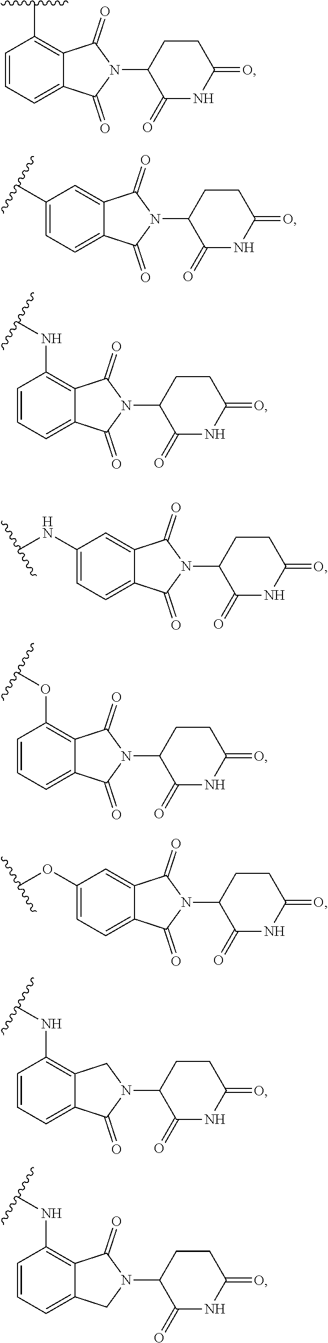

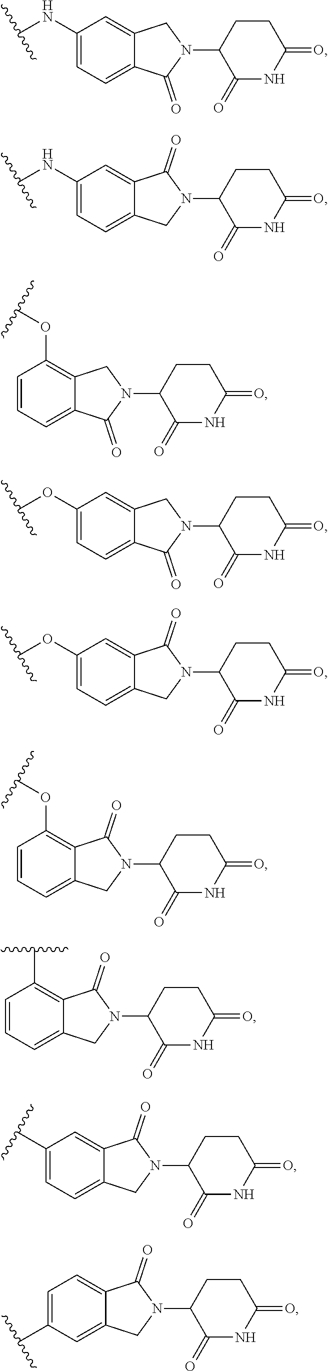

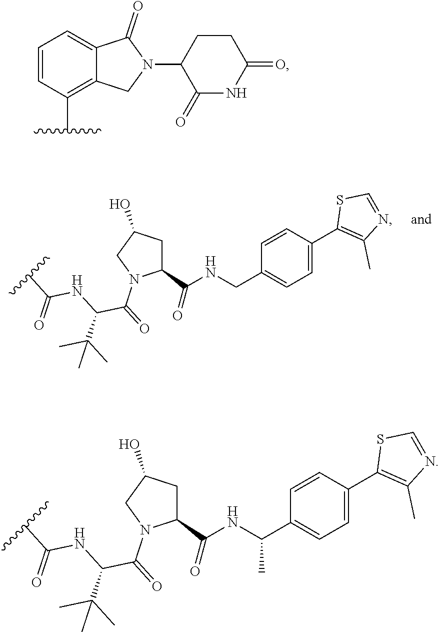 [SMILES: *c1cccc2c1CN(C1CCC(=O)NC1=O)C2=O ; *C(=O)N[C@H](C(=O)N1C[C@H](O)C[C@H]1C(=O)NCc1ccc(-c2scnc2C)cc1)C(C)(C)C ; "and" ; *C(=O)N[C@H](C(=O)N1C[C@H](O)C[C@H]1C(=O)N[C@@H](C)c1ccc(-c2scnc2C)cc1)C(C)(C)C]

In still another embodiment, a compound of the disclosure comprises Formula (II), wherein R¹ may be

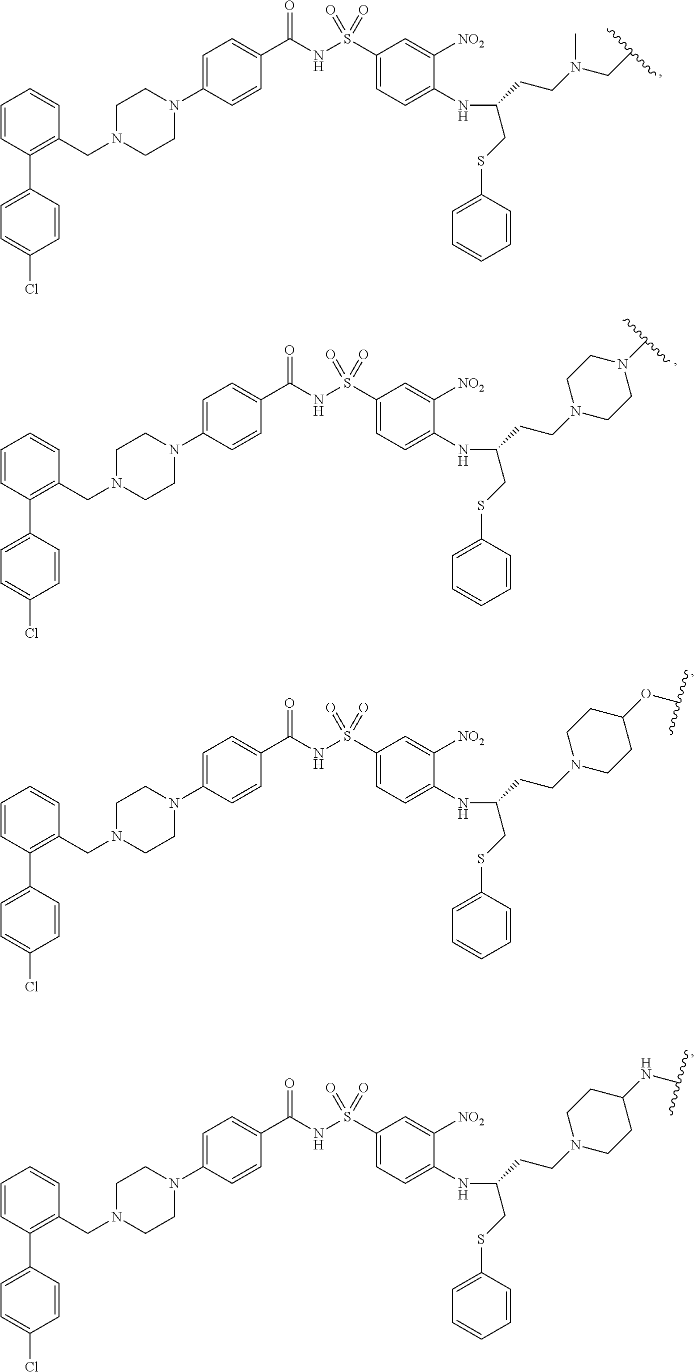 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

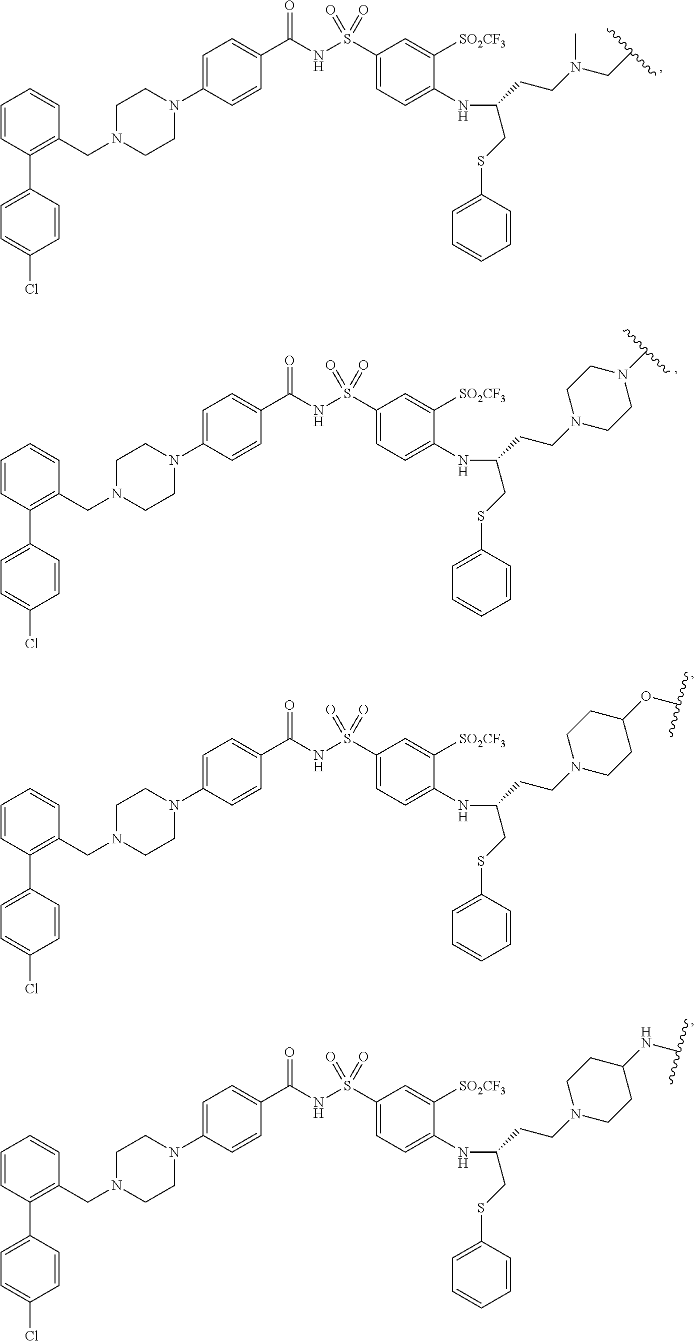

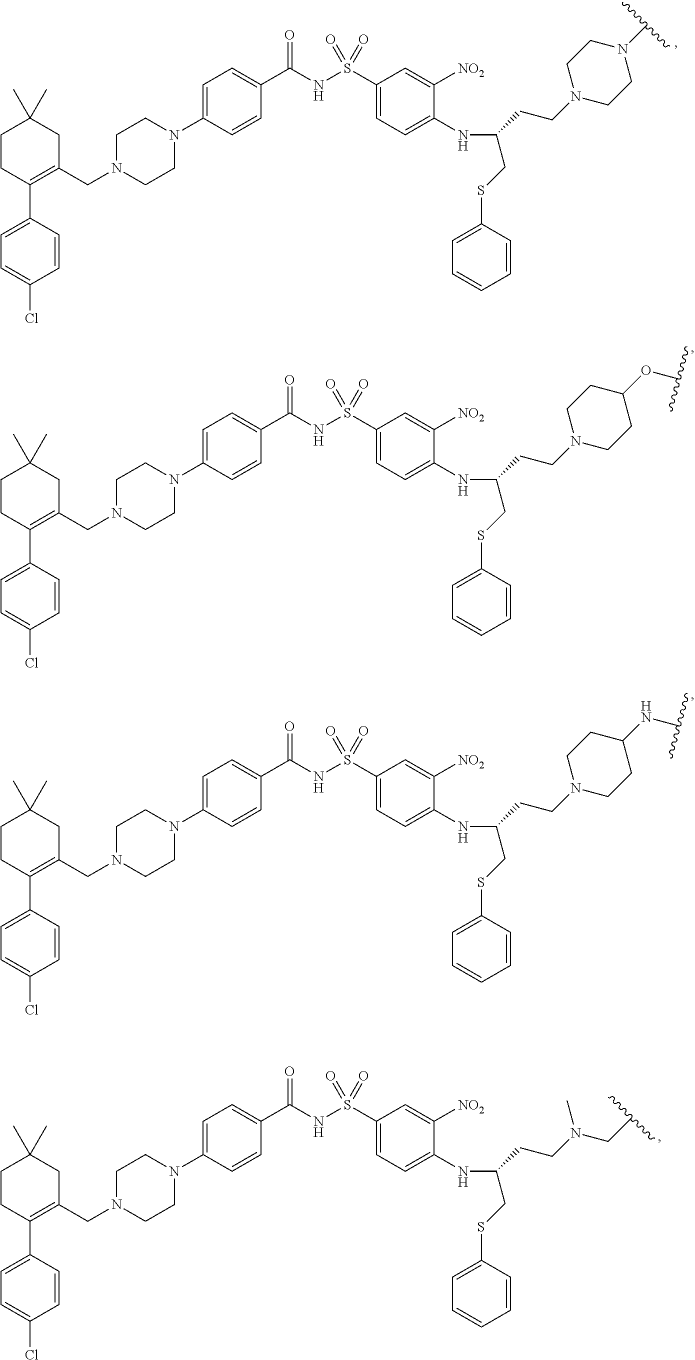 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

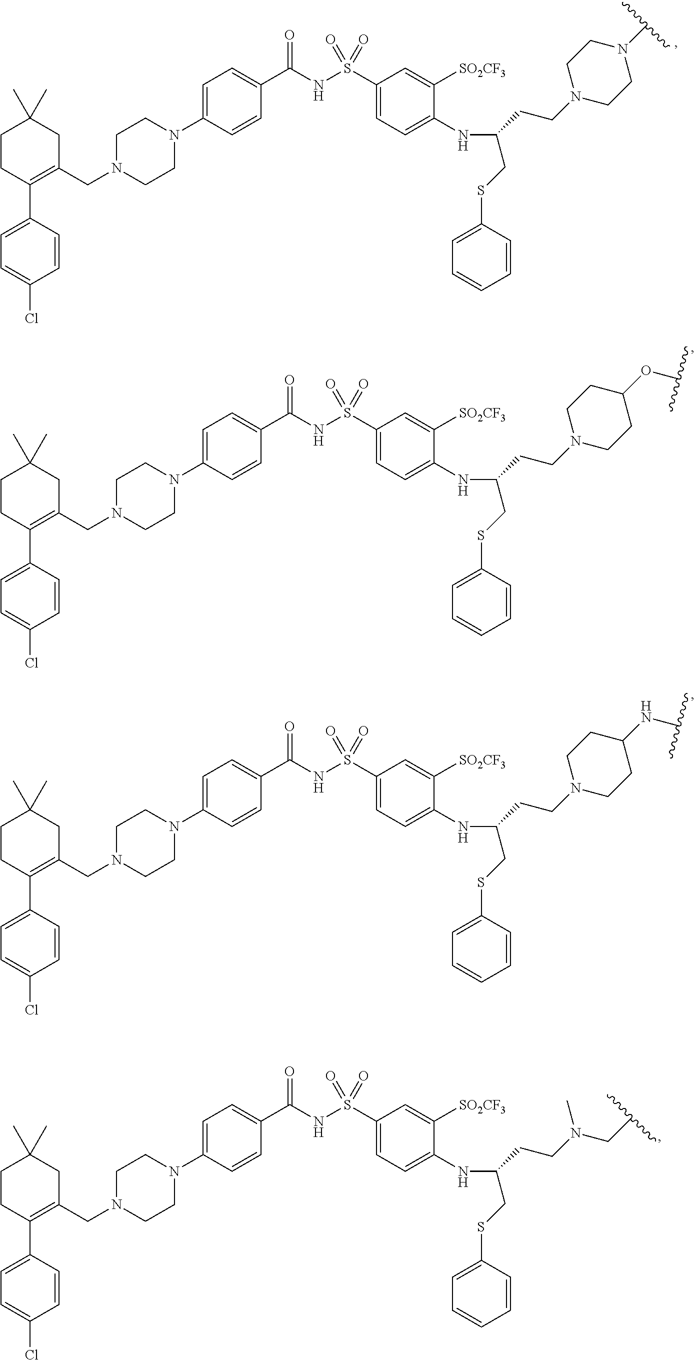

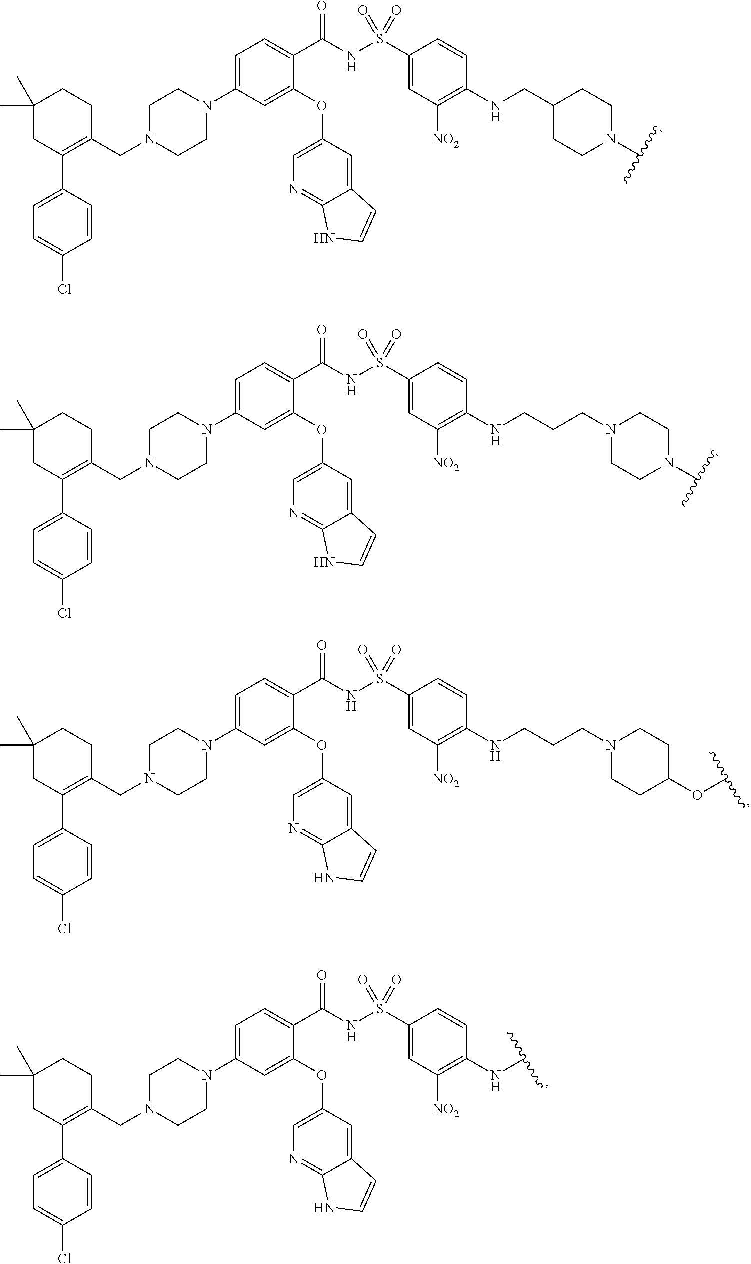 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *OC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

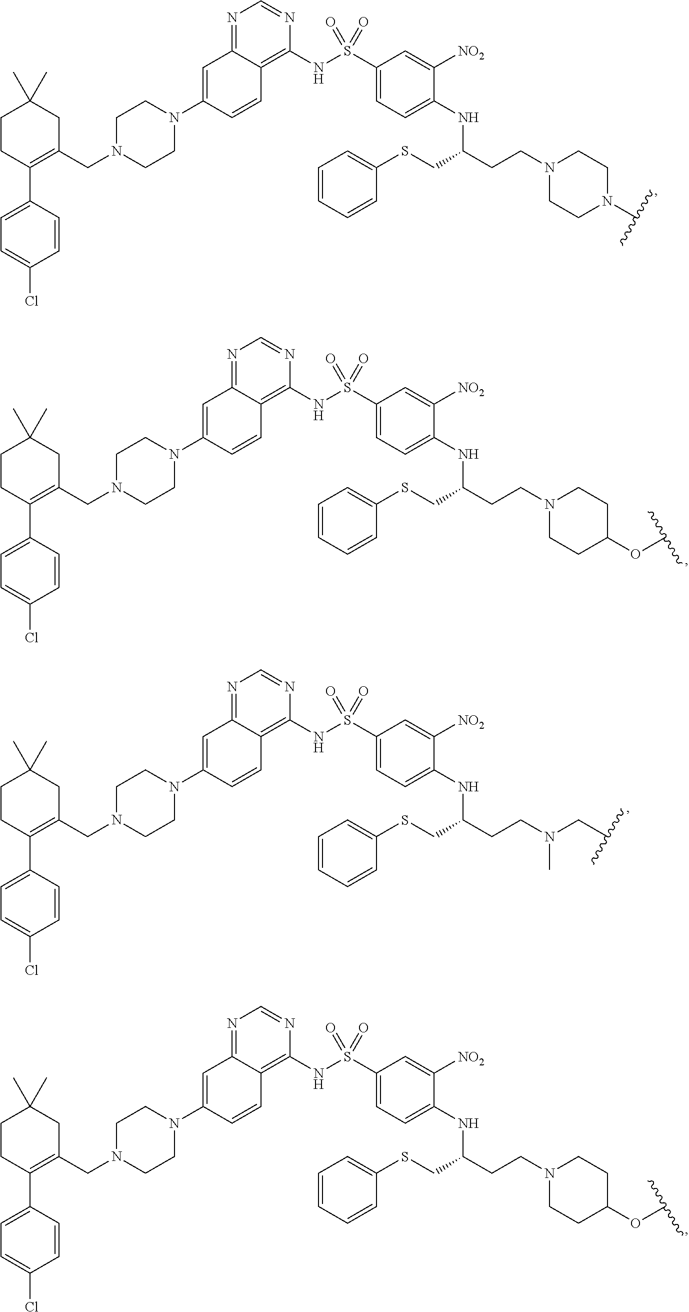 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

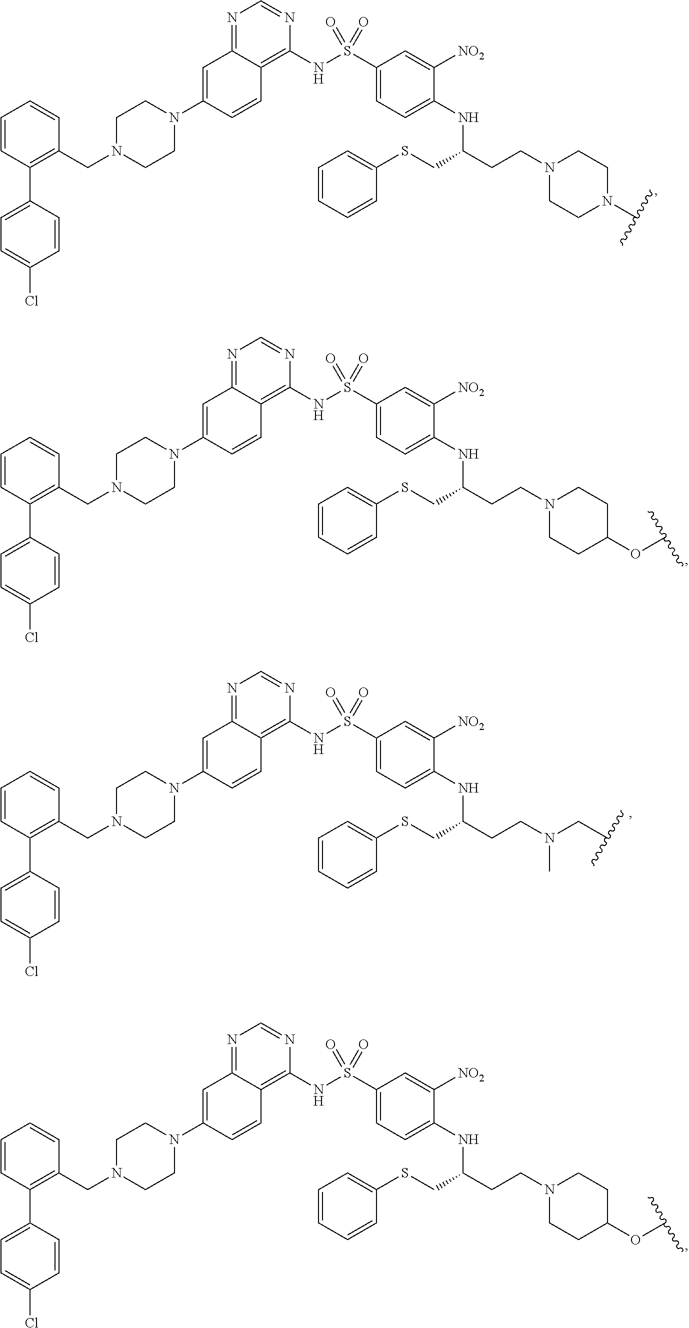 [SMILES: *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *CN(C)CC[C@H](CSc1ccccc1)Nc1ccc(S(=O)(=O)Nc2ncnc3cc(N4CCN(Cc5ccccc5-c5ccc(Cl)cc5)CC4)ccc23)cc1[N+](=O)[O-] ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

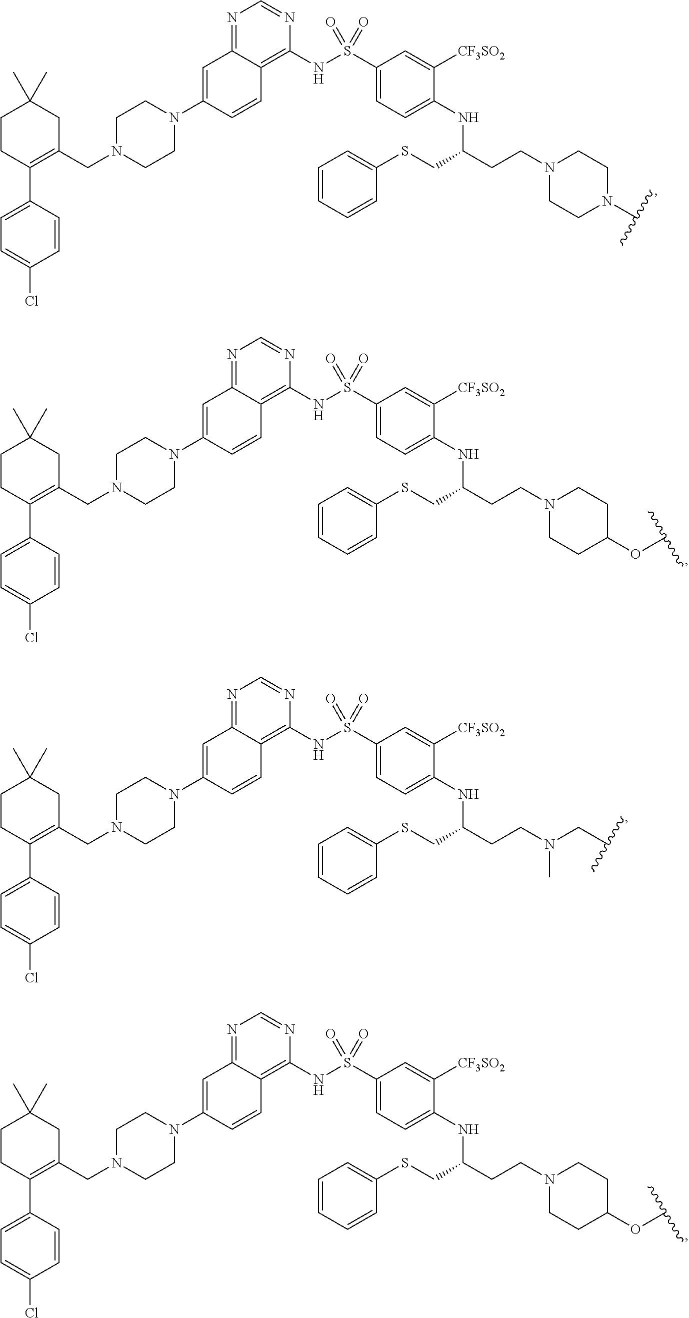

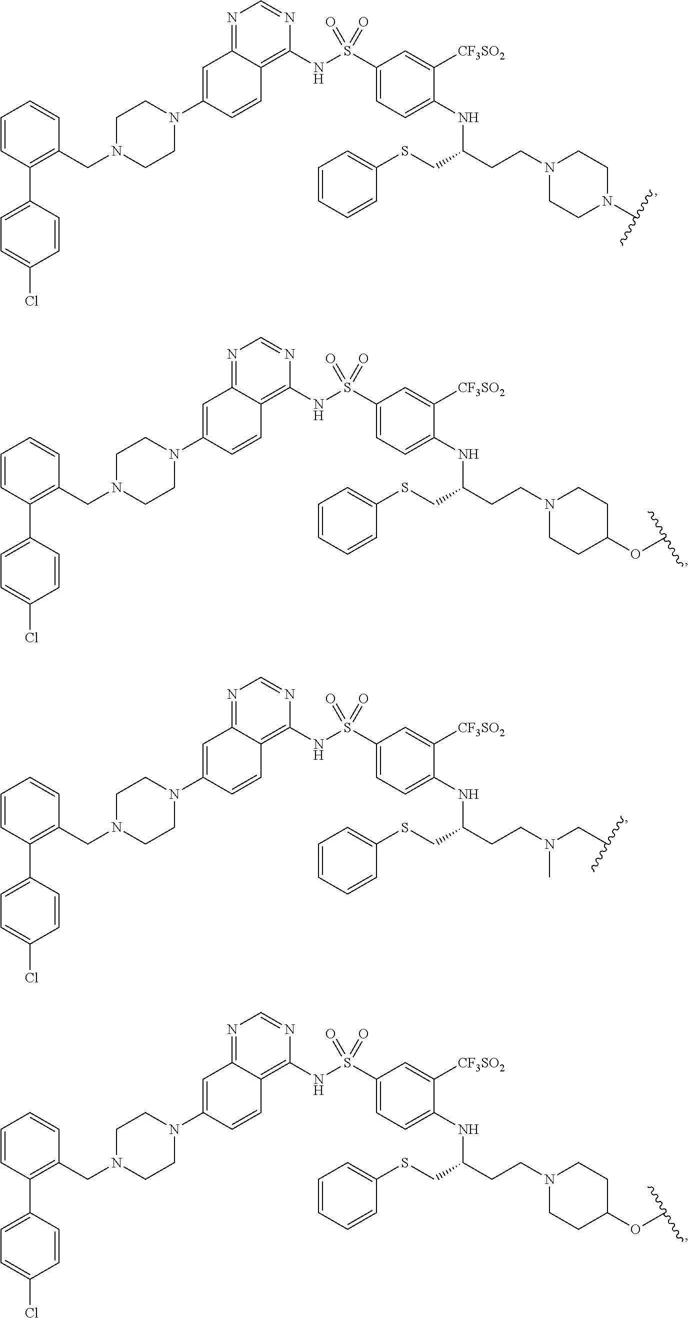

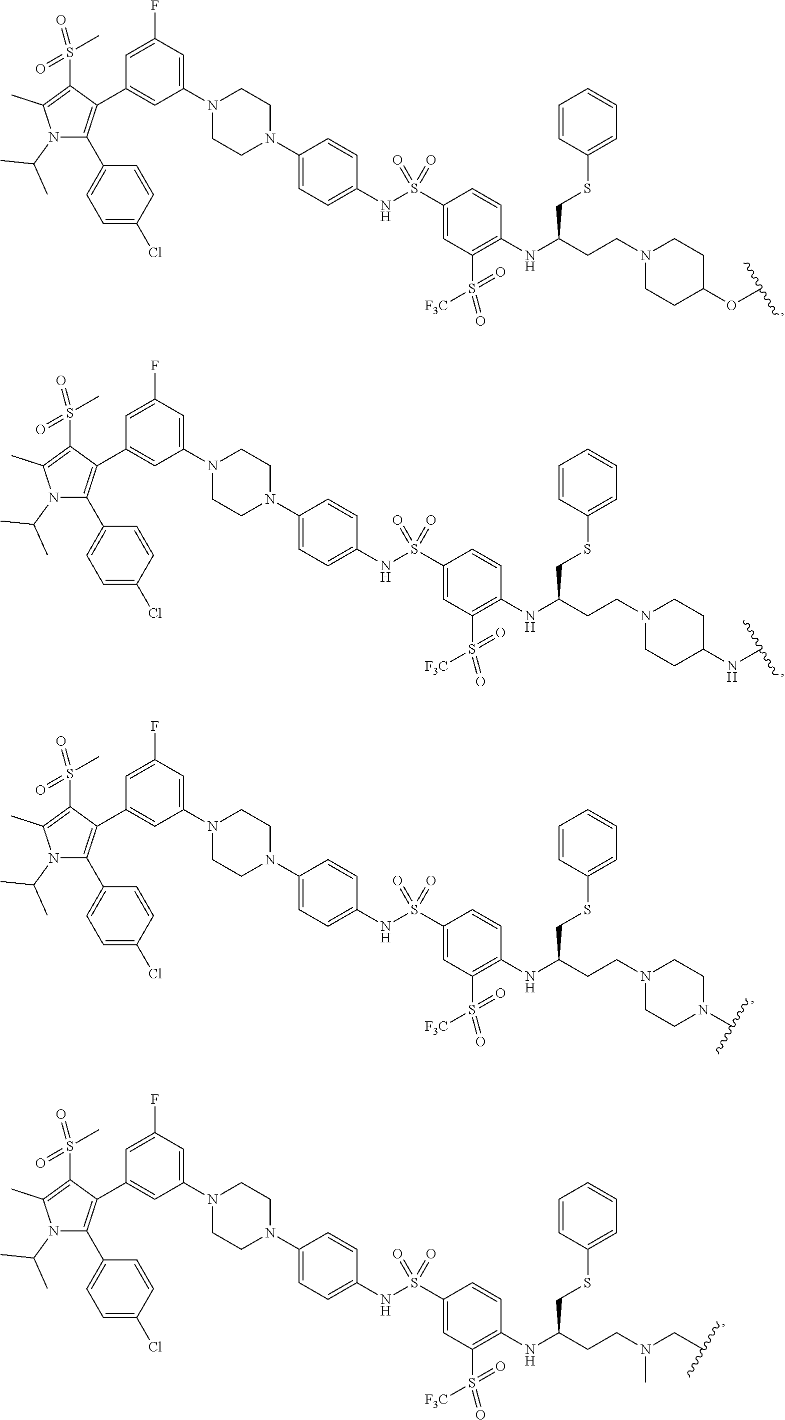

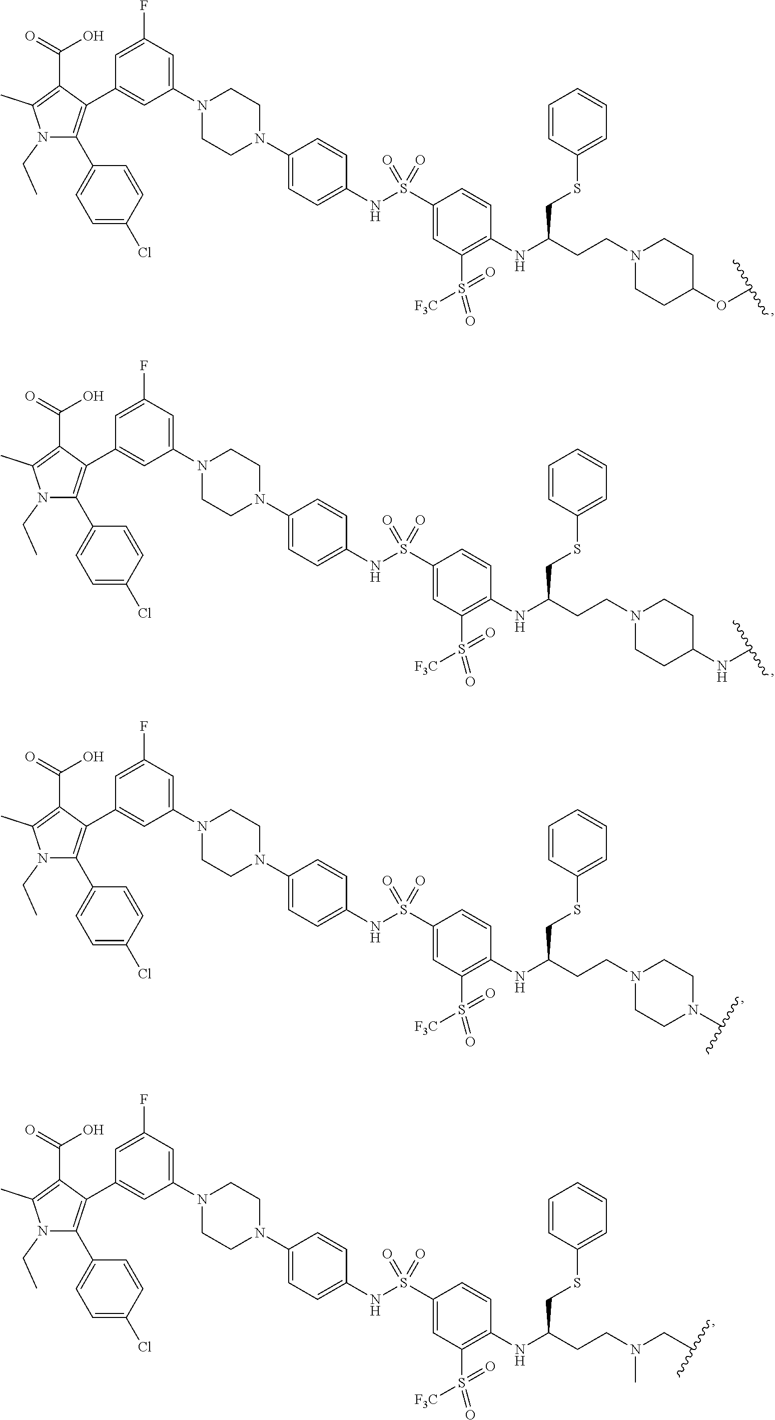

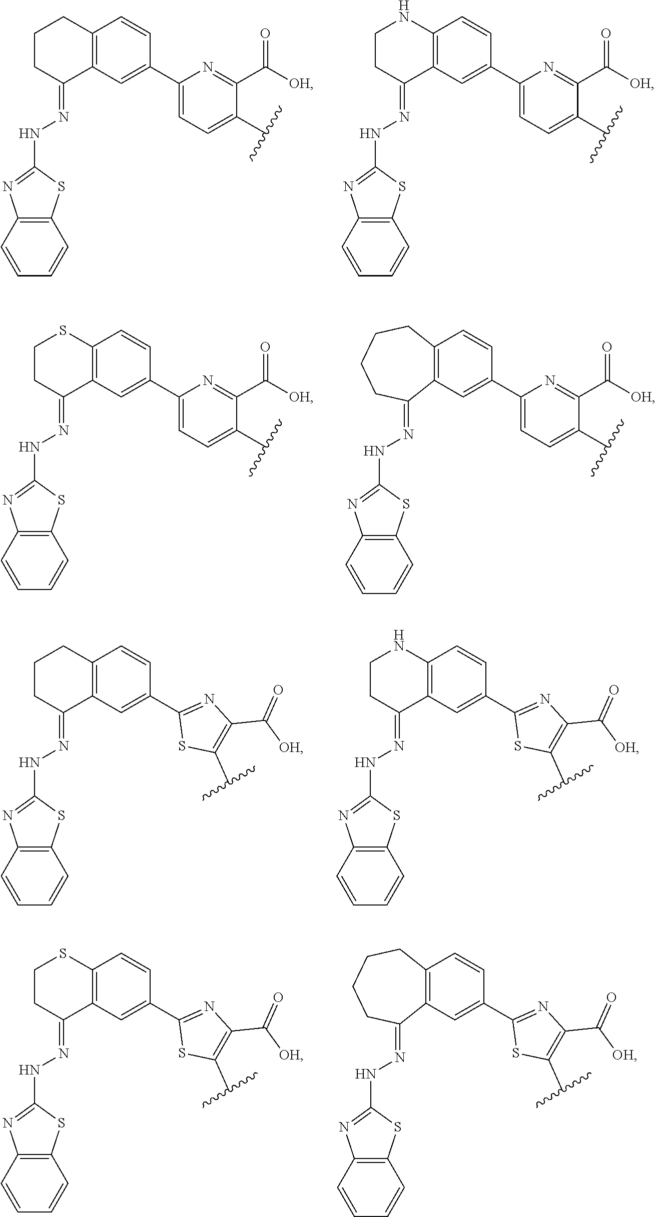

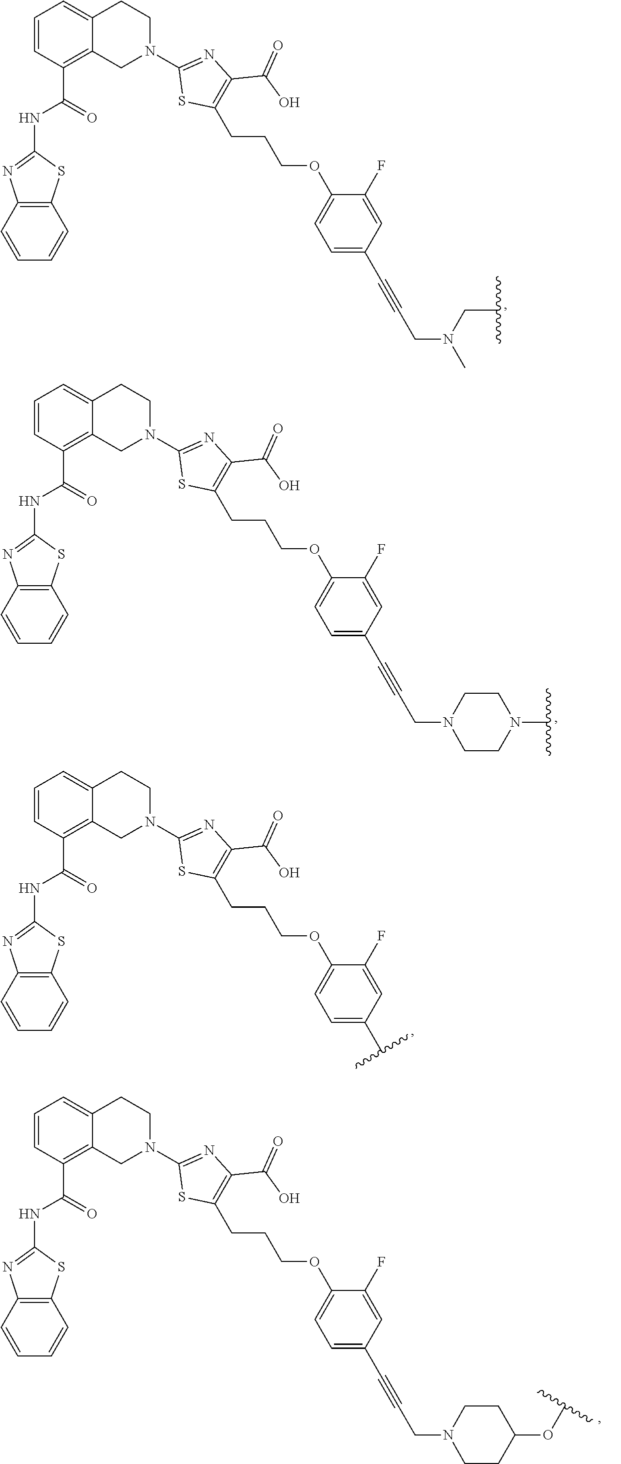 [SMILES: *CN(C)CC#Cc1ccc(OCCCc2sc(N3CCc4cccc(C(=O)Nc5nc6ccccc6s5)c4C3)nc2C(=O)O)c(F)c1 ; *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1]

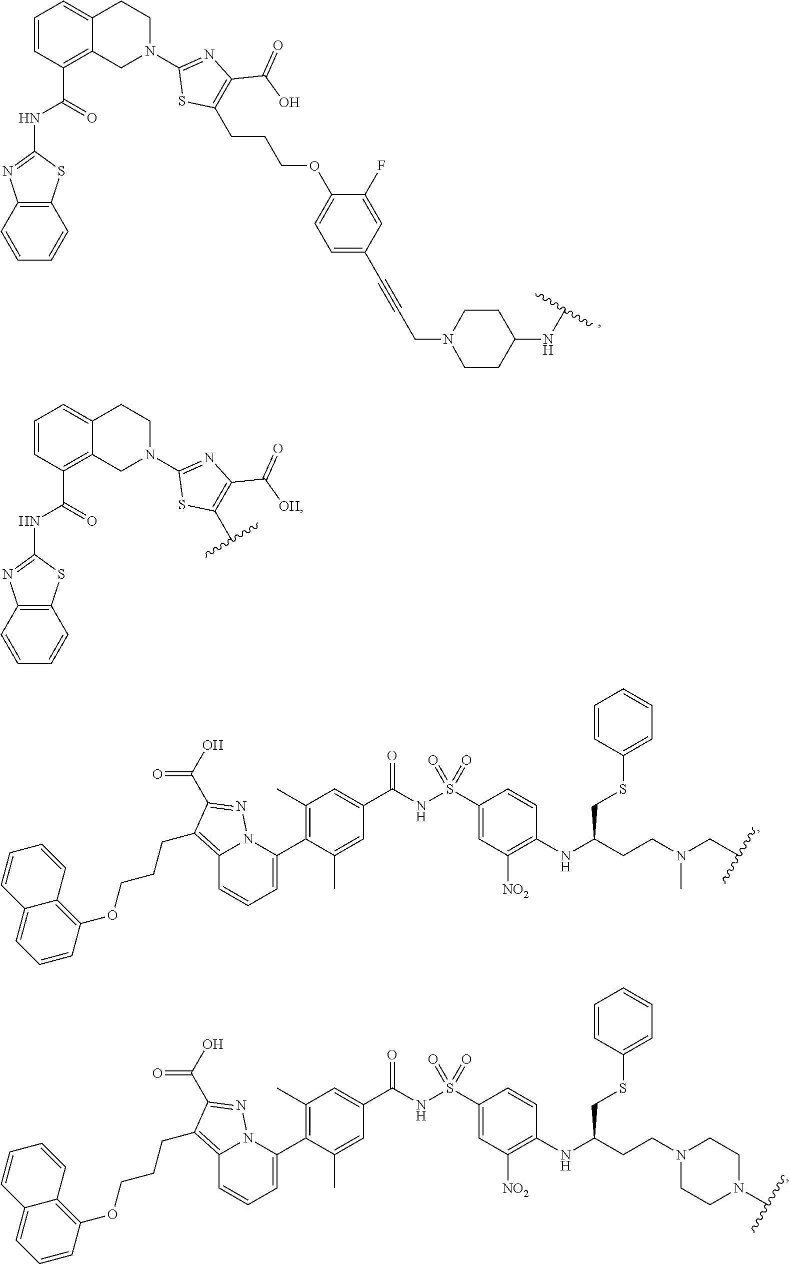 [SMILES: *NC1CCN(CC#Cc2ccc(OCCCc3sc(N4CCc5cccc(C(=O)Nc6nc7ccccc7s6)c5C4)nc3C(=O)O)c(F)c2)CC1 ; *c1sc(N2CCc3cccc(C(=O)Nc4nc5ccccc5s4)c3C2)nc1C(=O)O ; *CN(C)CC[C@H](CSc1ccccc1)Nc1ccc(S(=O)(=O)NC(=O)c2cc(C)c(-c3cccc4c(CCCOc5cccc6ccccc56)c(C(=O)O)nn34)c(C)c2)cc1[N+](=O)[O-] ; *N1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1]

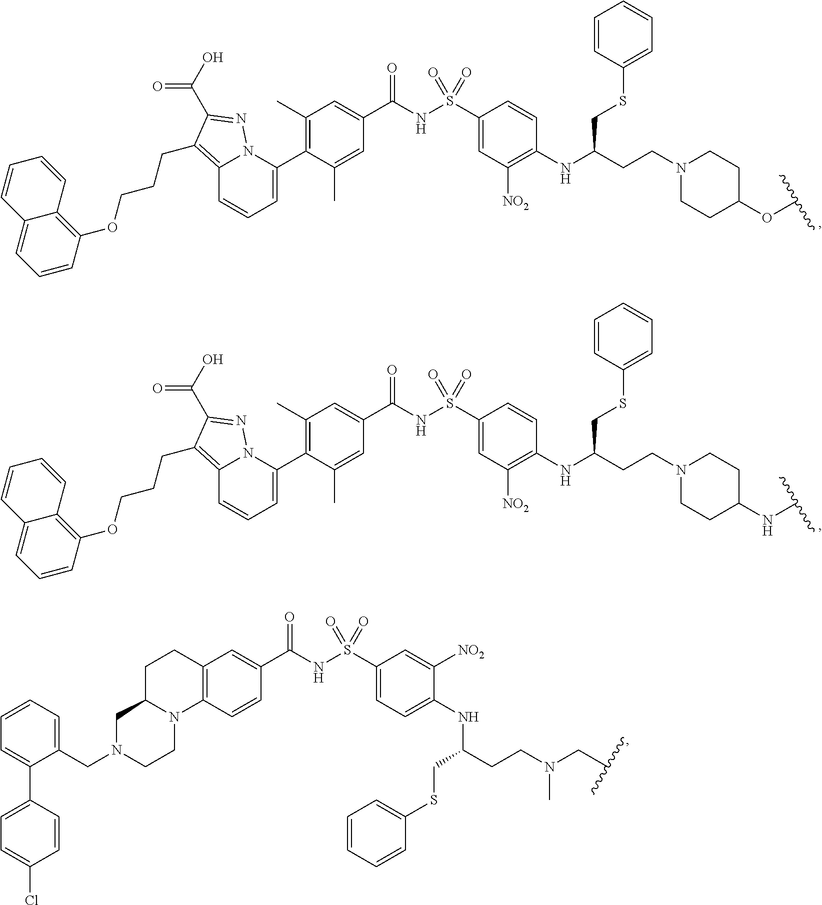 [SMILES: *OC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *NC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-]]

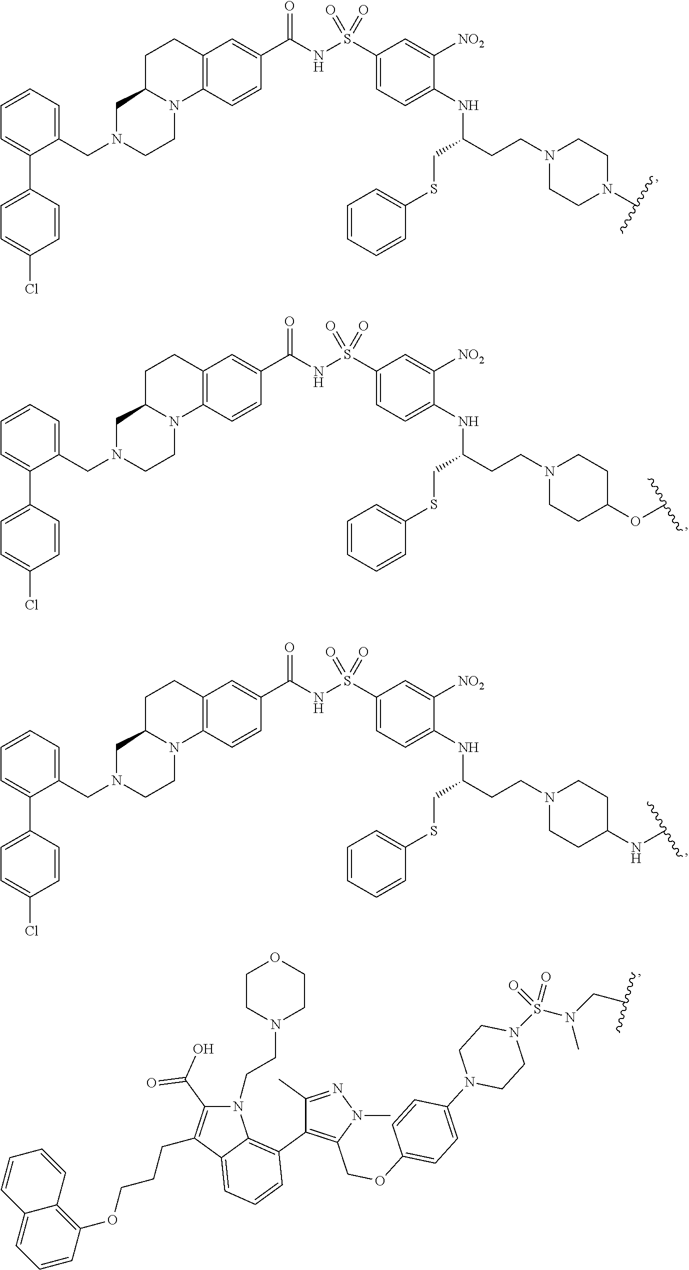 [SMILES: *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1]

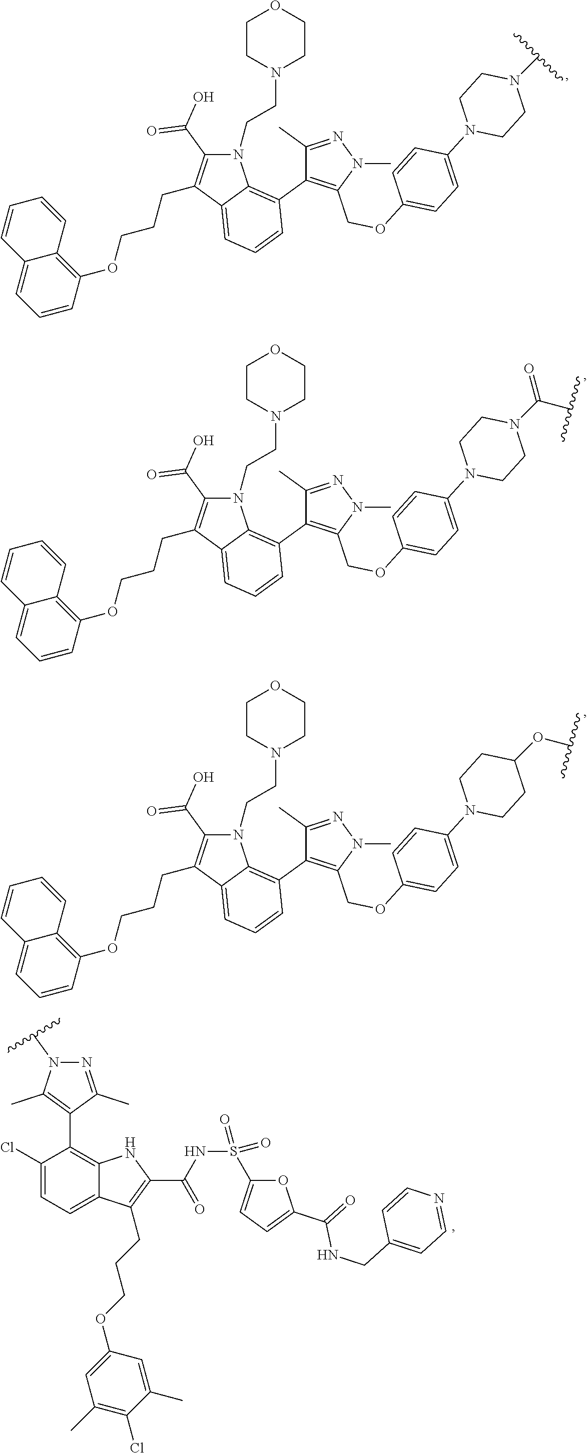

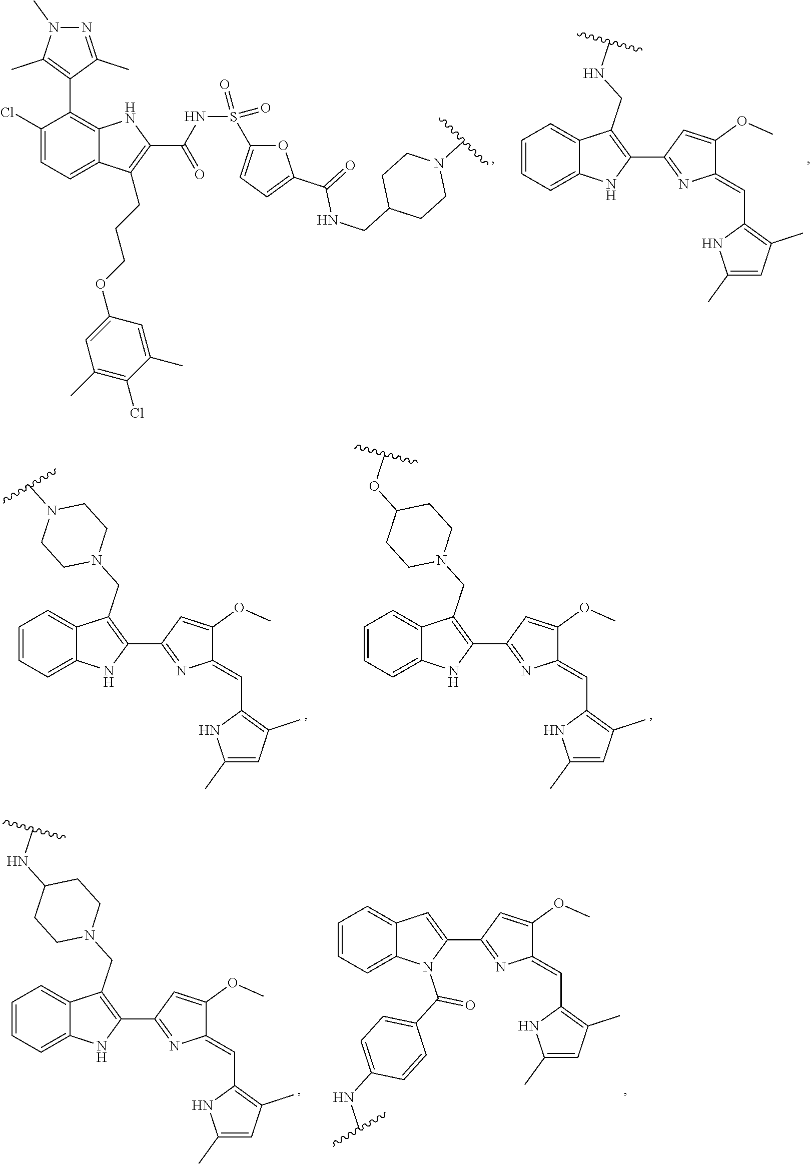 [SMILES: *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12 ; *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1 ; *NC1CCN(Cc2c(C3=N/C(=C\c4[nH]c(C)cc4C)C(OC)=C3)[nH]c3ccccc23)CC1 ; *Nc1ccc(C(=O)n2c(C3=N/C(=C\c4[nH]c(C)cc4C)C(OC)=C3)cc3ccccc32)cc1]

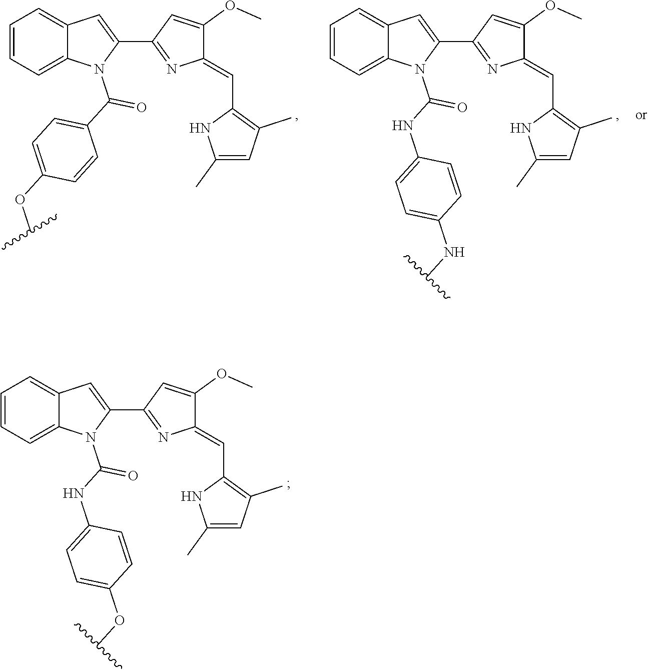 [SMILES: *Oc1ccc(C(=O)n2c(C3=N/C(=C\c4[nH]c(C)cc4C)C(OC)=C3)cc3ccccc32)cc1 ; *Nc1ccc(NC(=O)n2c(C3=N/C(=C\c4[nH]c(C)cc4C)C(OC)=C3)cc3ccccc32)cc1 ; *Oc1ccc(NC(=O)n2c(C3=N/C(=C\c4[nH]c(C)cc4C)C(OC)=C3)cc3ccccc32)cc1]

R³ may be absent, an unsubstituted C₁-C₆ alkyl, or a substituted or unsubstituted C₃-C₆ ketone; A may be absent, a bond, or a substituted or unsubstituted C₁-C₆ heterocyclic group; n may be 0 to 3; R⁴ may be a bond or a substituted C₁-C₁₀ alkyl, and R² may be

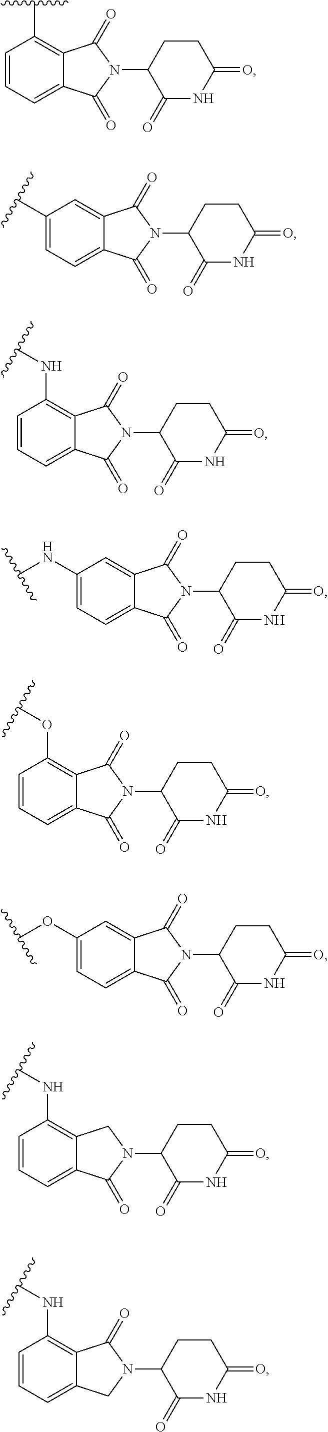

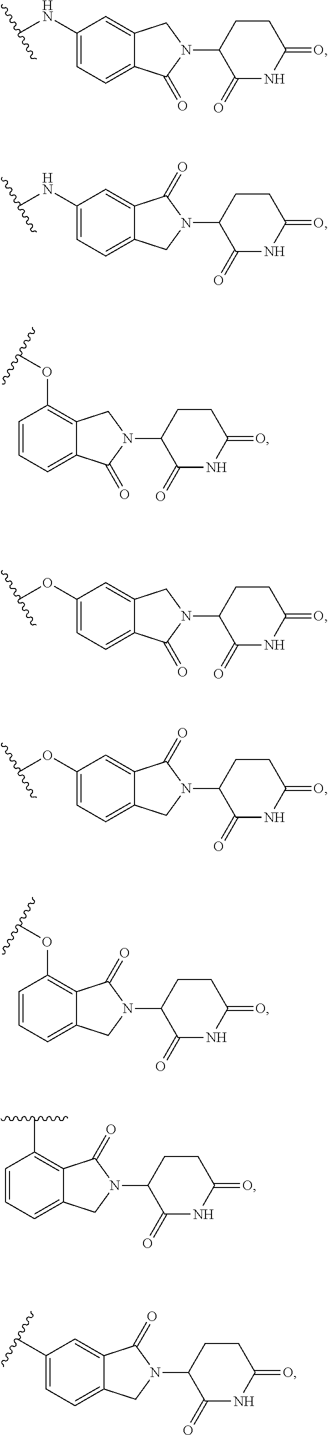

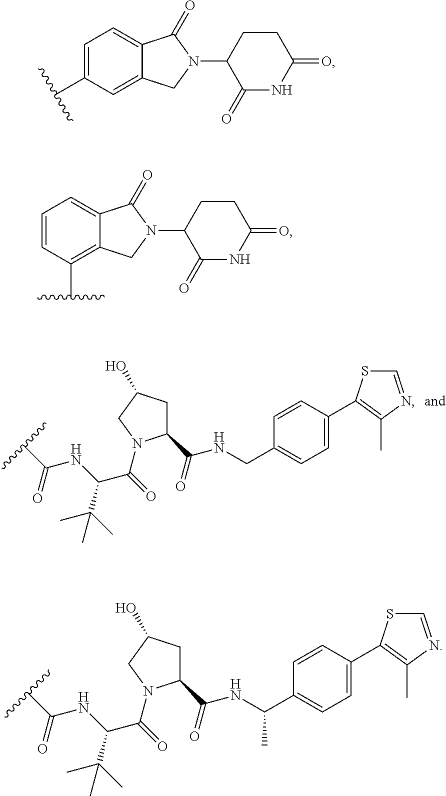 [SMILES: *c1ccc2c(c1)CN(C1CCC(=O)NC1=O)C2=O ; *c1cccc2c1CN(C1CCC(=O)NC1=O)C2=O ; *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

In a different embodiment, a compound of the disclosure comprises Formula (II), wherein R¹ may be

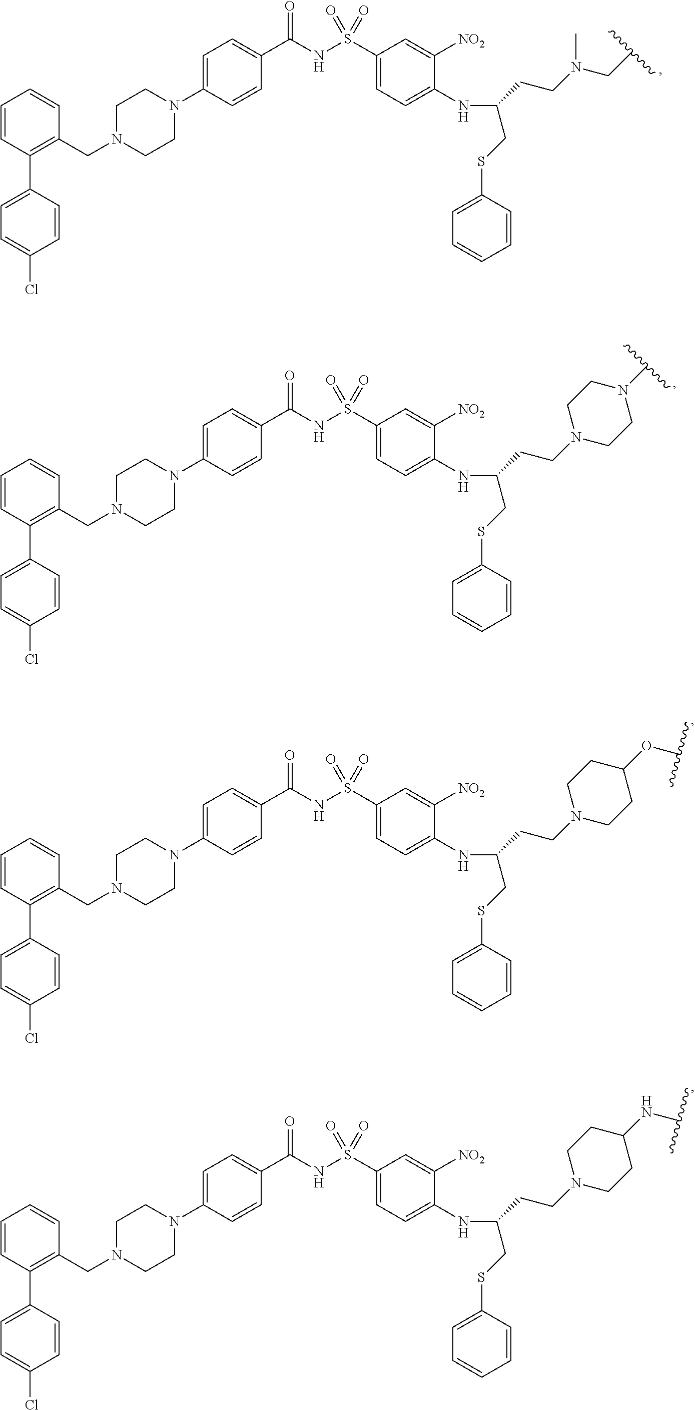 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

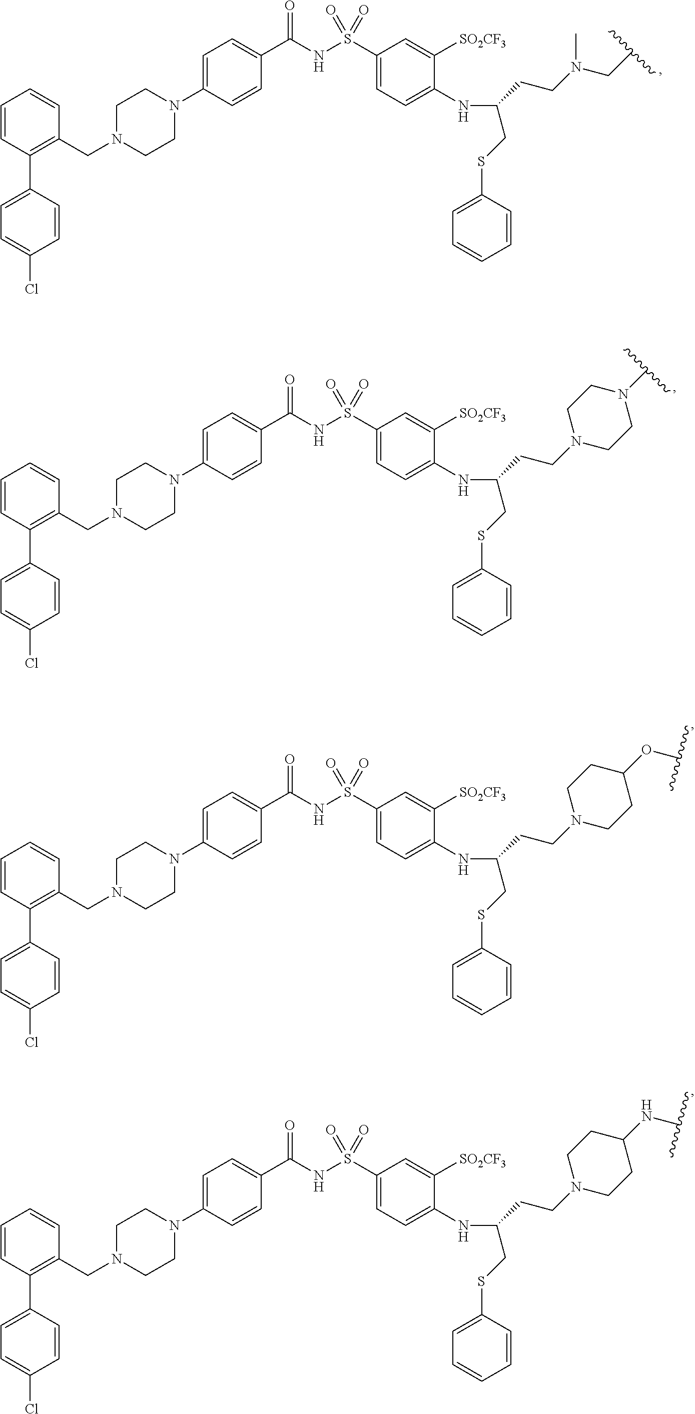

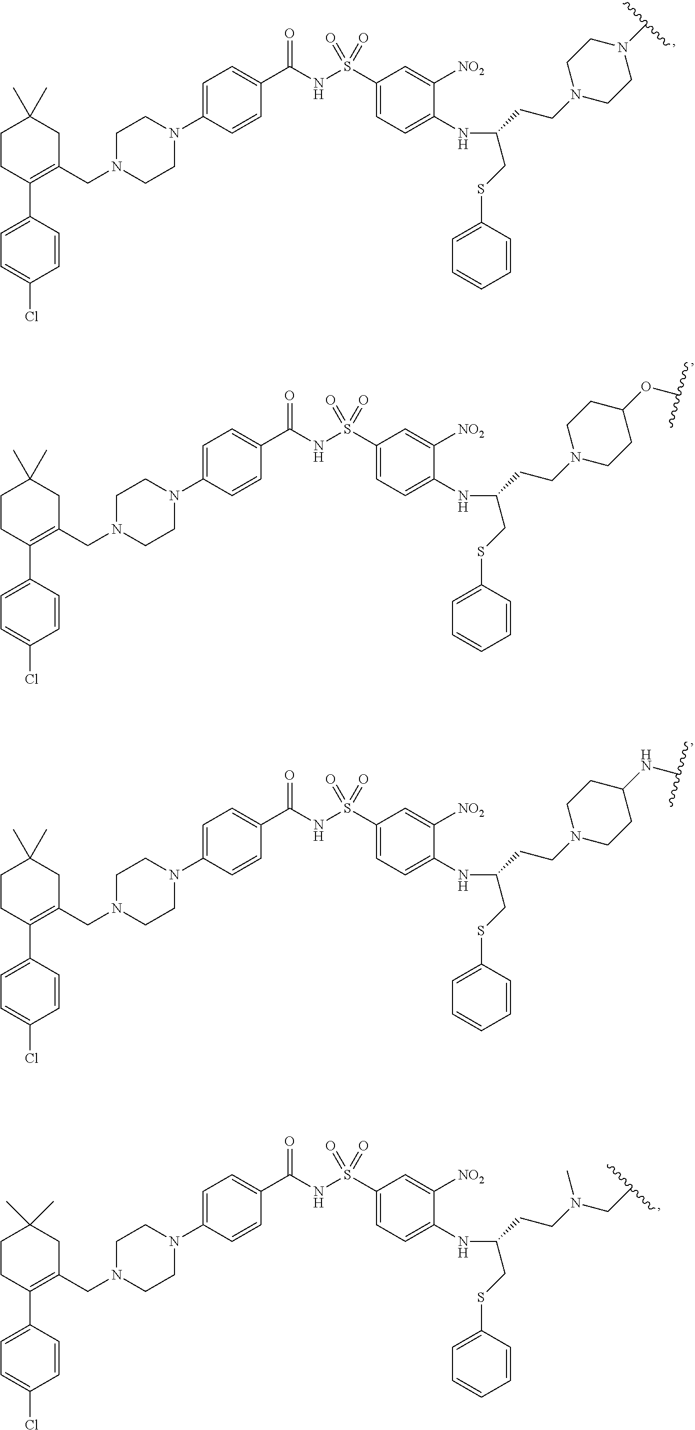 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]]

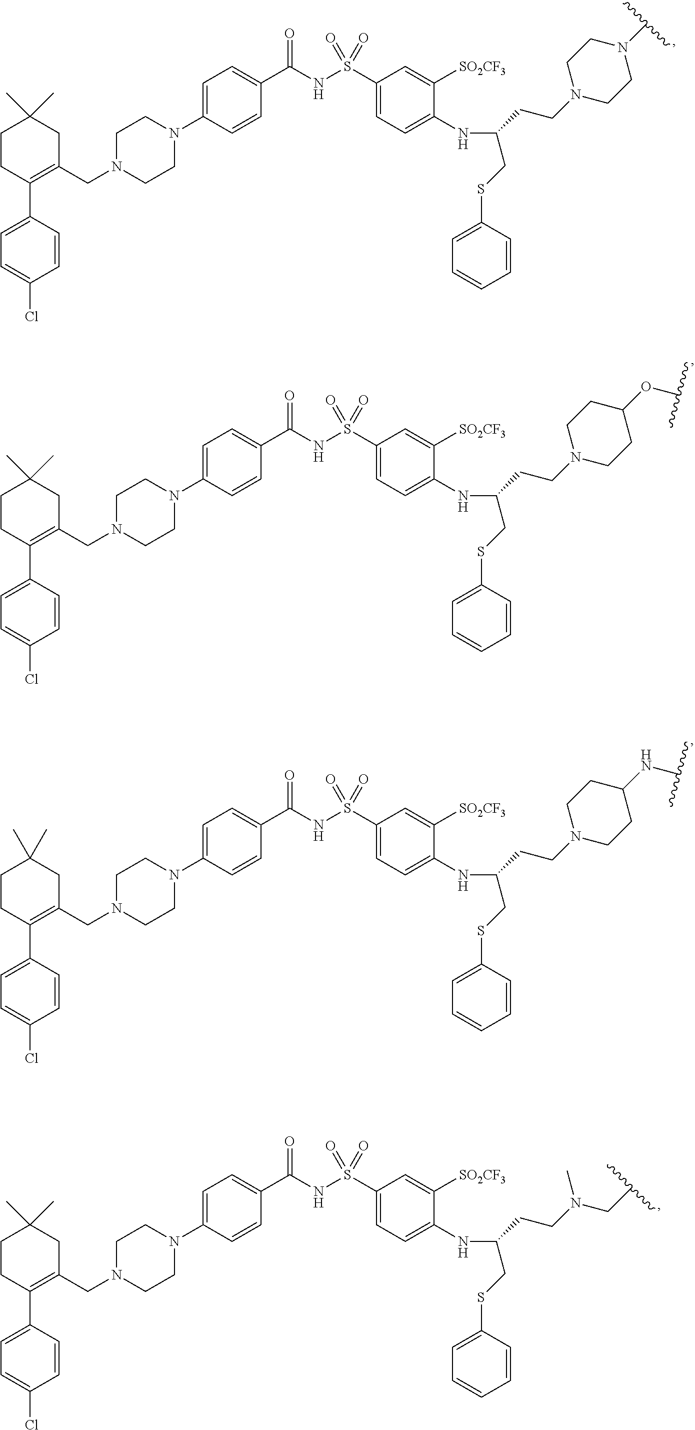

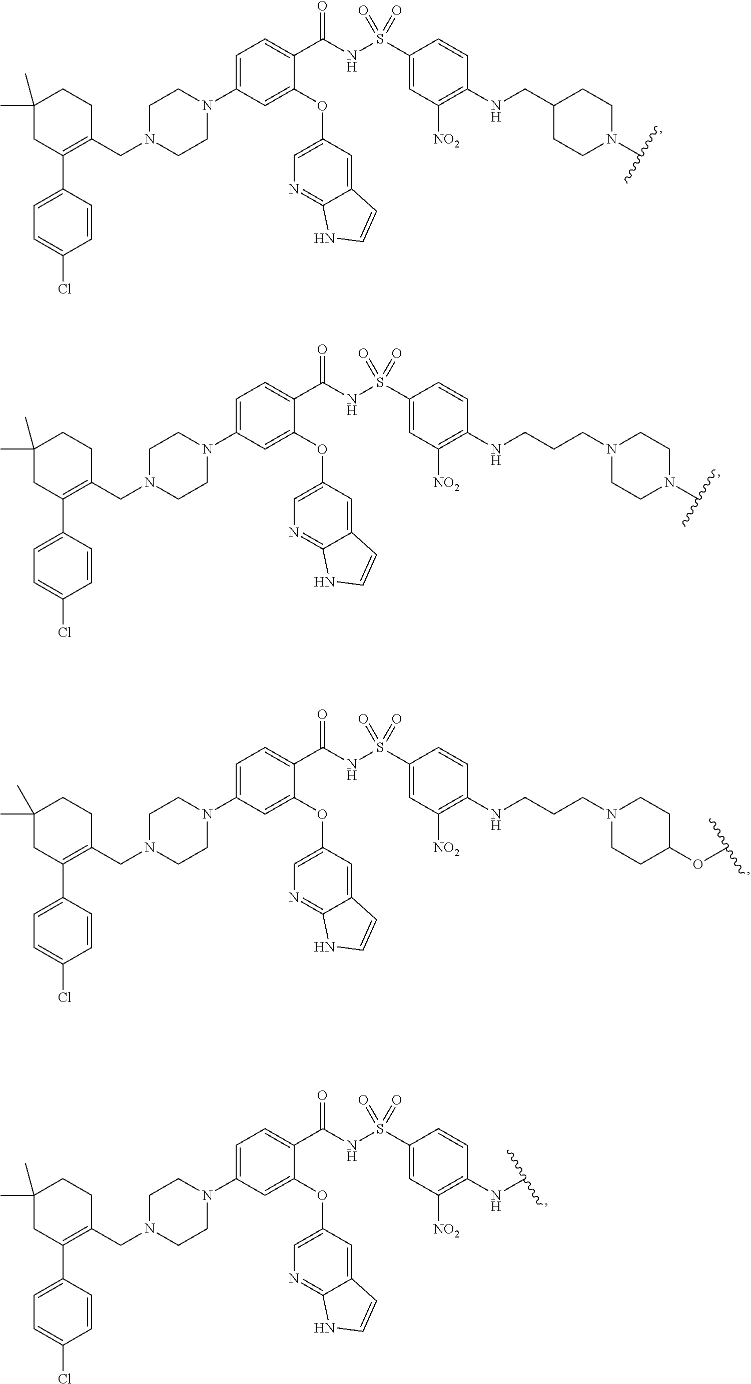 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *OC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

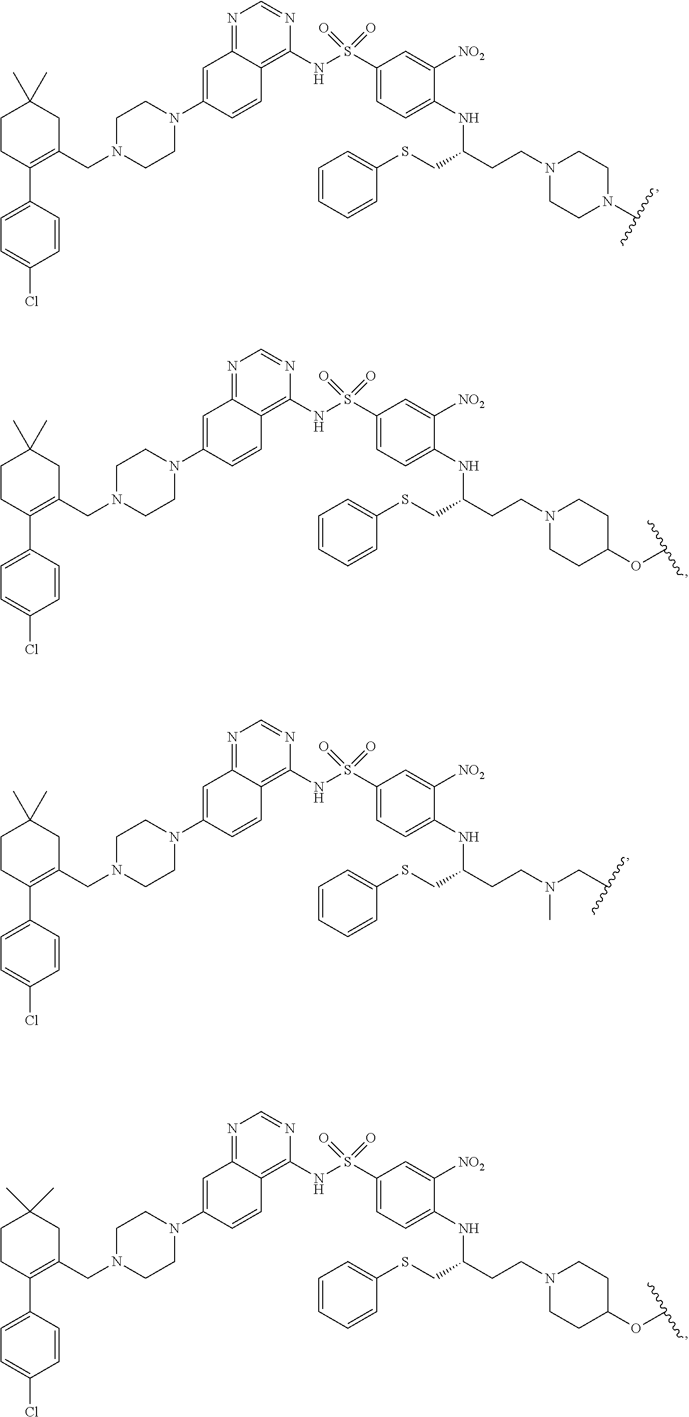 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1]

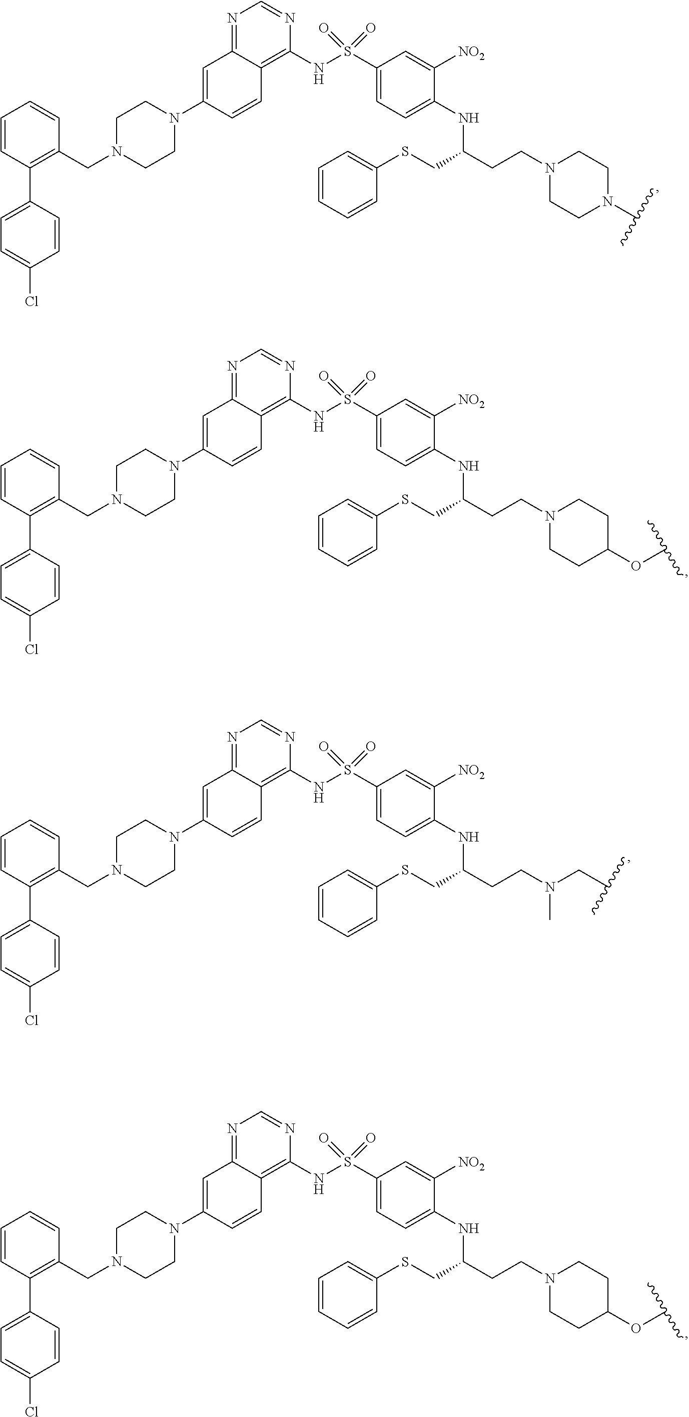 [SMILES: *N1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1 ; *CN(C)CC[C@H](CSc1ccccc1)Nc1ccc(S(=O)(=O)Nc2ncnc3cc(N4CCN(Cc5ccccc5-c5ccc(Cl)cc5)CC4)ccc23)cc1[N+](=O)[O-] ; *OC1CCN(CC[C@H](CSc2ccccc2)Nc2ccc(S(=O)(=O)Nc3ncnc4cc(N5CCN(Cc6ccccc6-c6ccc(Cl)cc6)CC5)ccc34)cc2[N+](=O)[O-])CC1]

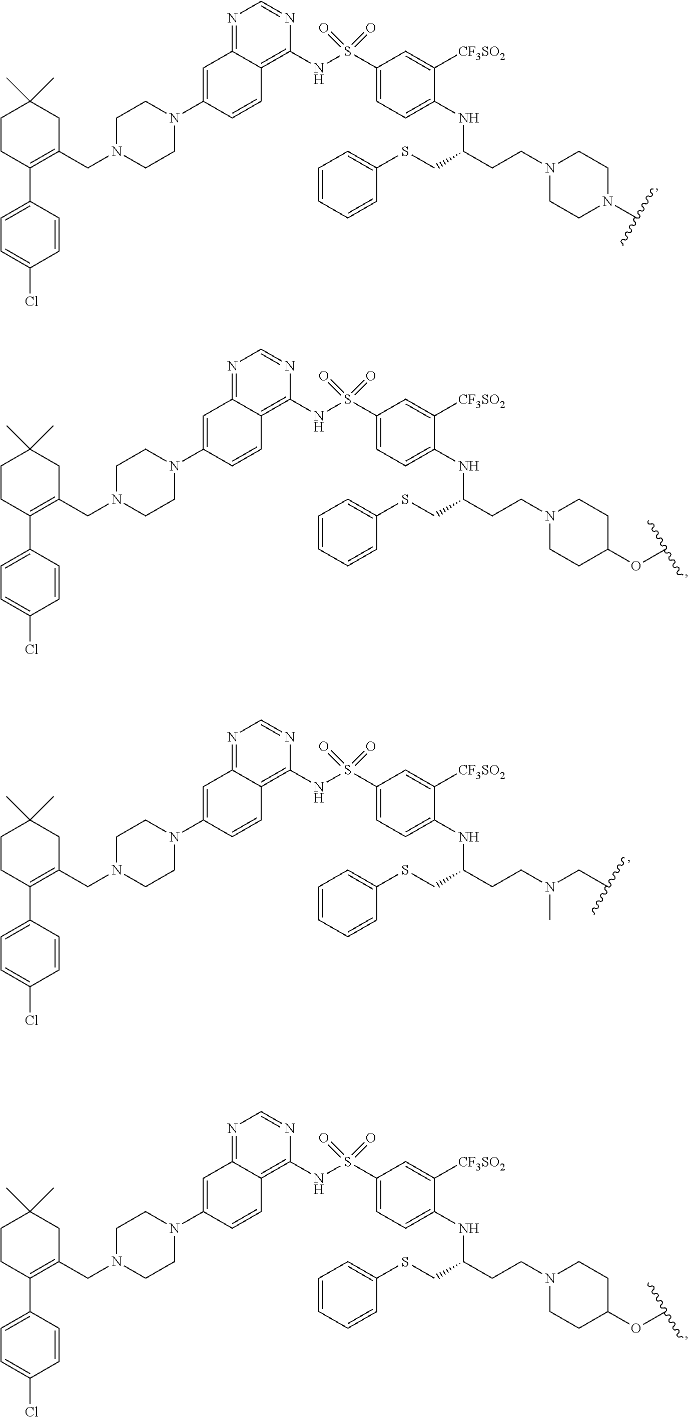

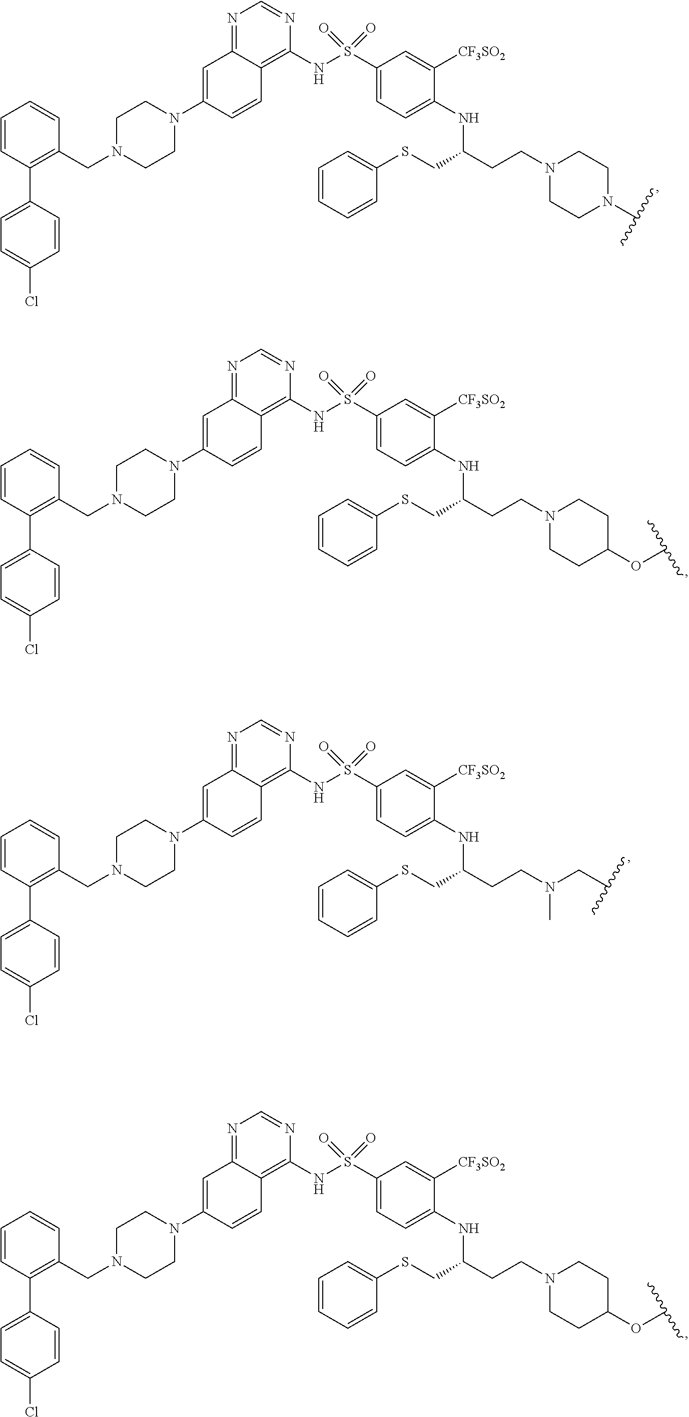

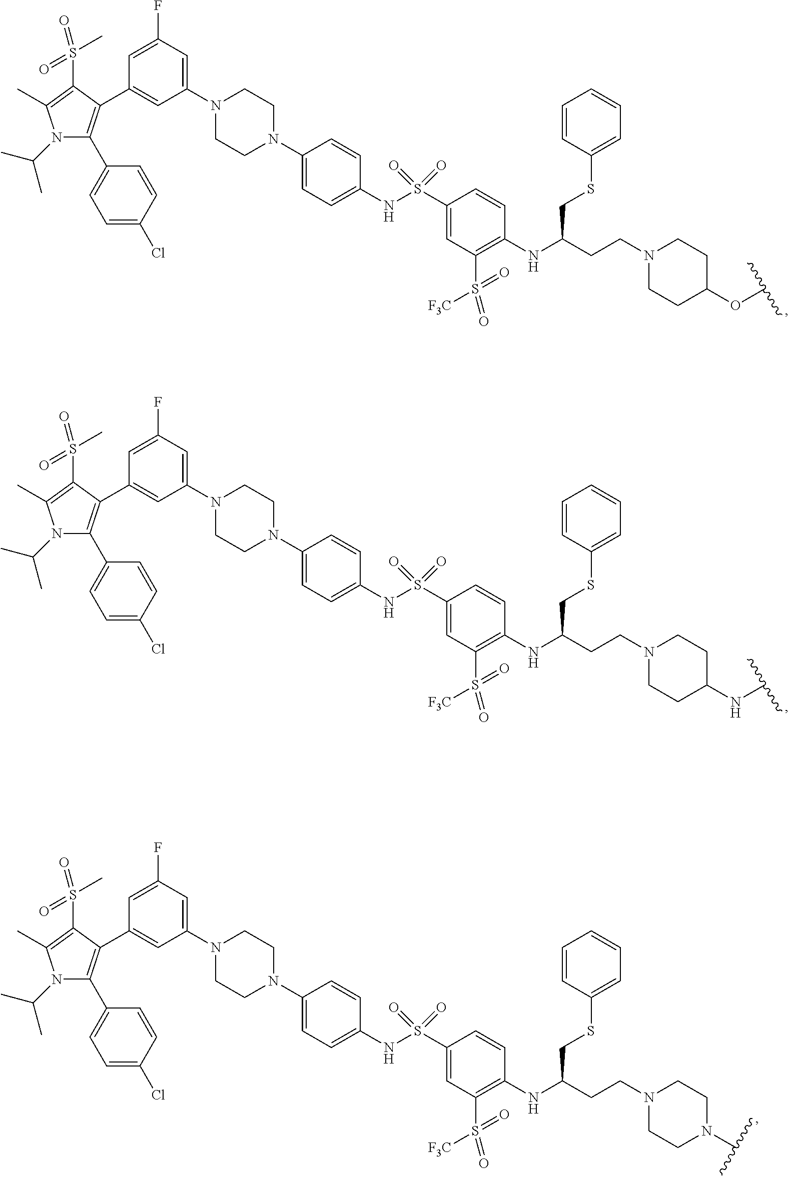 [SMILES: *OC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *N1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

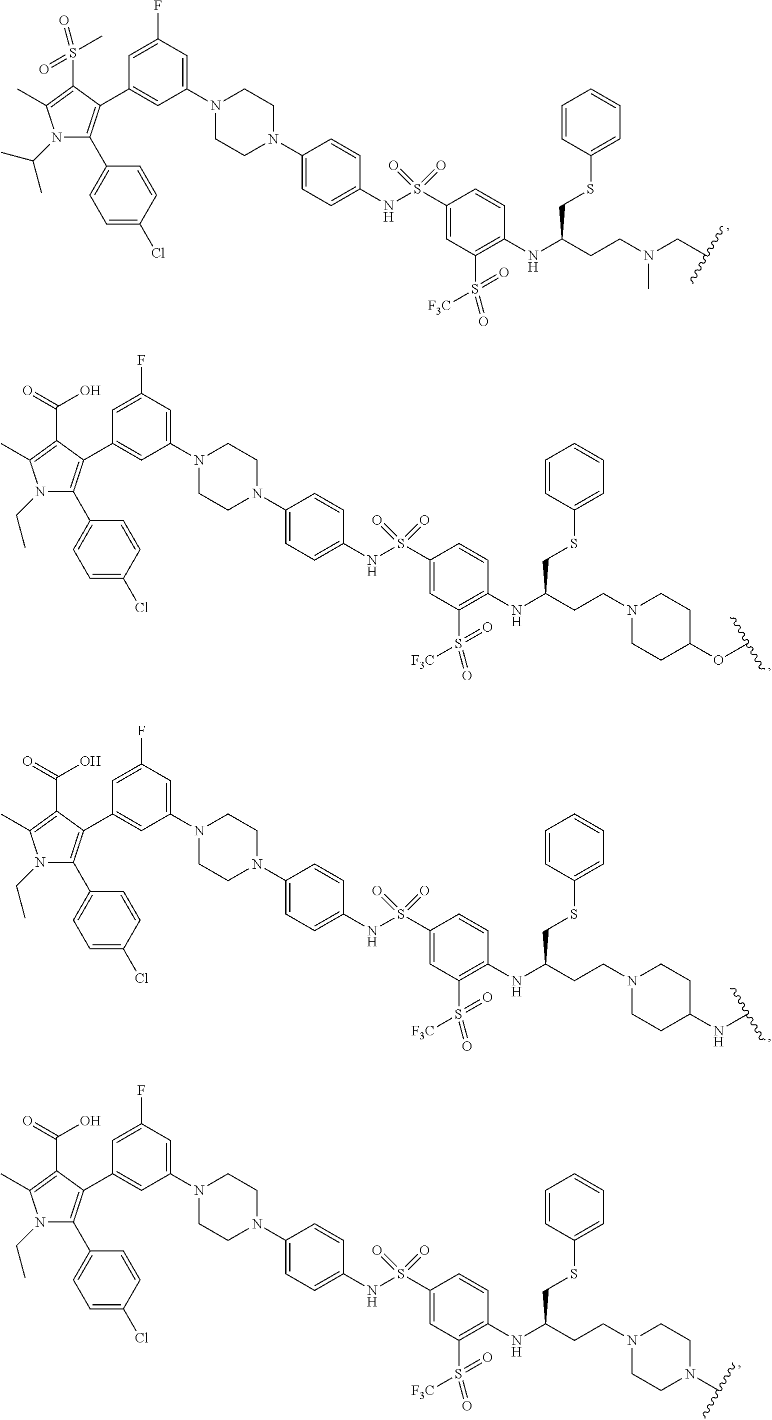

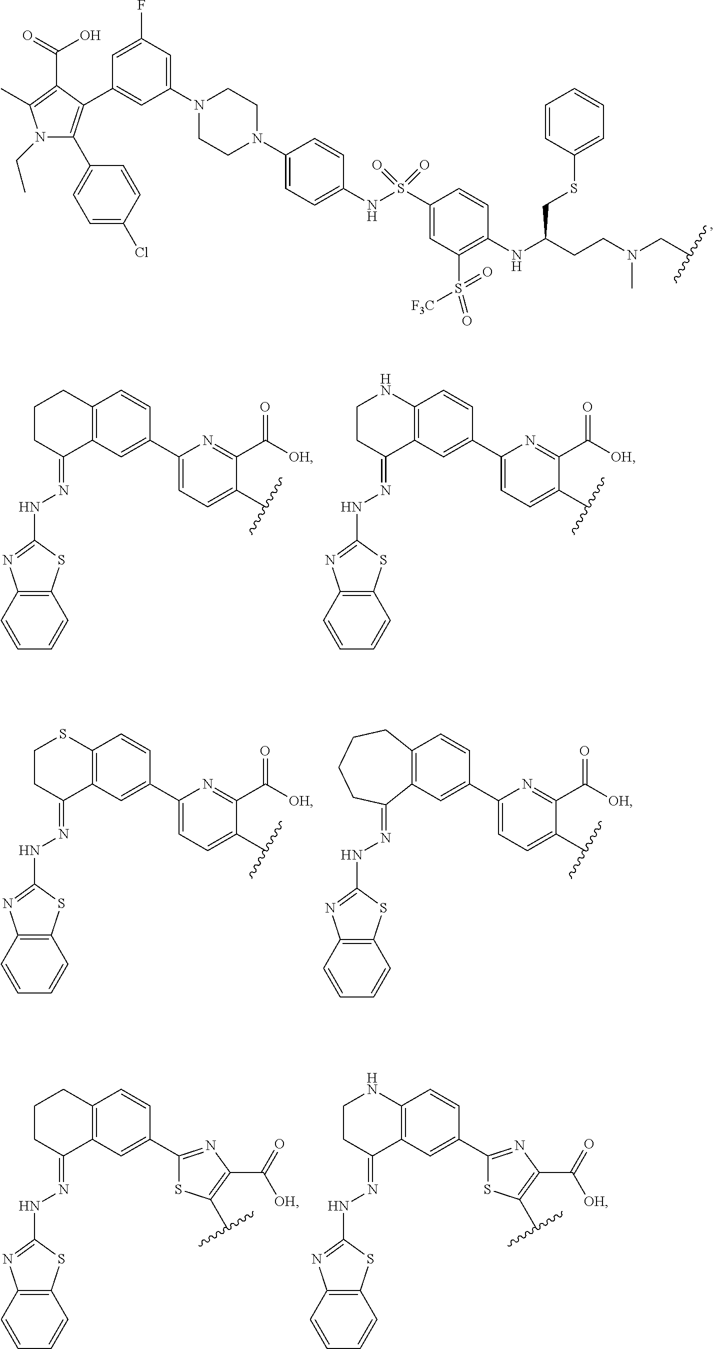

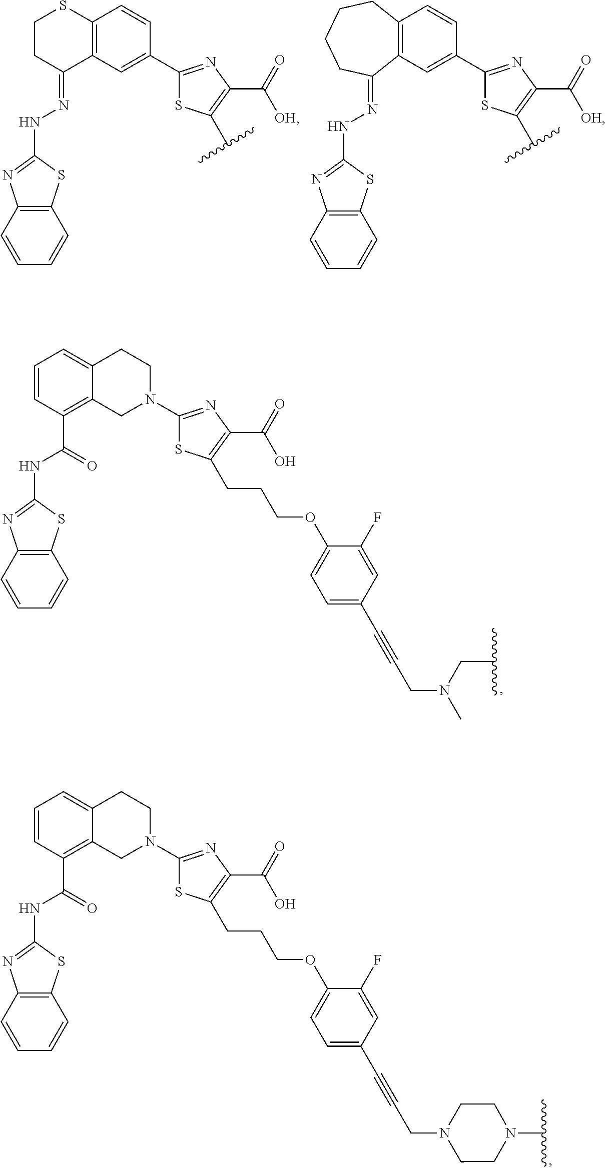 [SMILES: *c1sc(-c2ccc3c(c2)/C(=N/Nc2nc4ccccc4s2)CCS3)nc1C(=O)O ; *c1sc(-c2ccc3c(c2)/C(=N/Nc2nc4ccccc4s2)CCCC3)nc1C(=O)O ; *CN(C)CC#Cc1ccc(OCCCc2sc(N3CCc4cccc(C(=O)Nc5nc6ccccc6s5)c4C3)nc2C(=O)O)c(F)c1 ; *N1CCN(CC#Cc2ccc(OCCCc3sc(N4CCc5cccc(C(=O)Nc6nc7ccccc7s6)c5C4)nc3C(=O)O)c(F)c2)CC1]

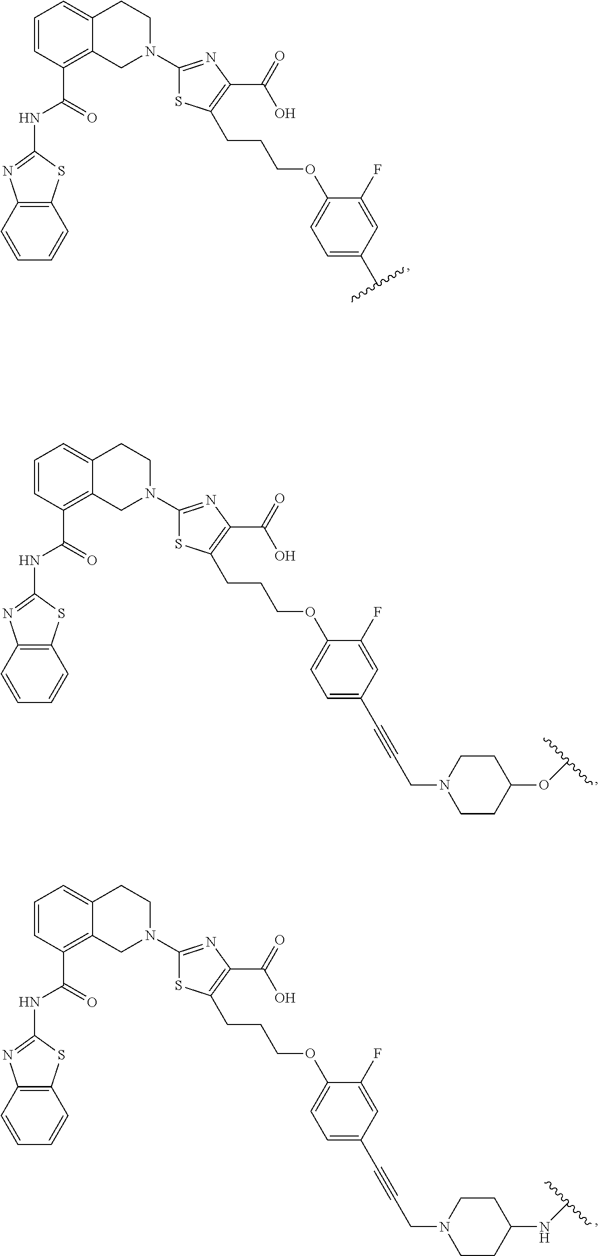 [SMILES: *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1 ; *NC1CCN(CC#Cc2ccc(OCCCc3sc(N4CCc5cccc(C(=O)Nc6nc7ccccc7s6)c5C4)nc3C(=O)O)c(F)c2)CC1]

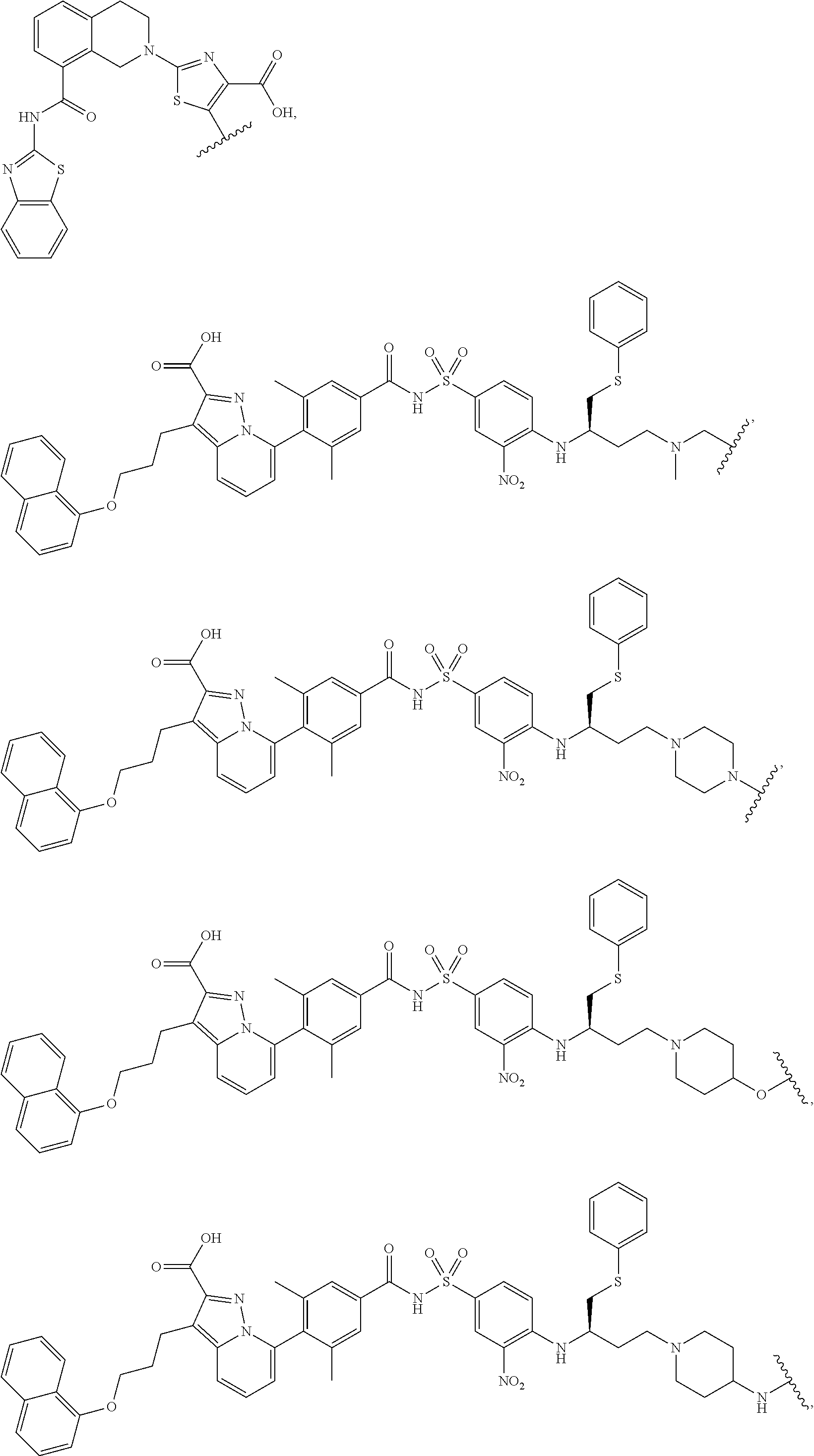

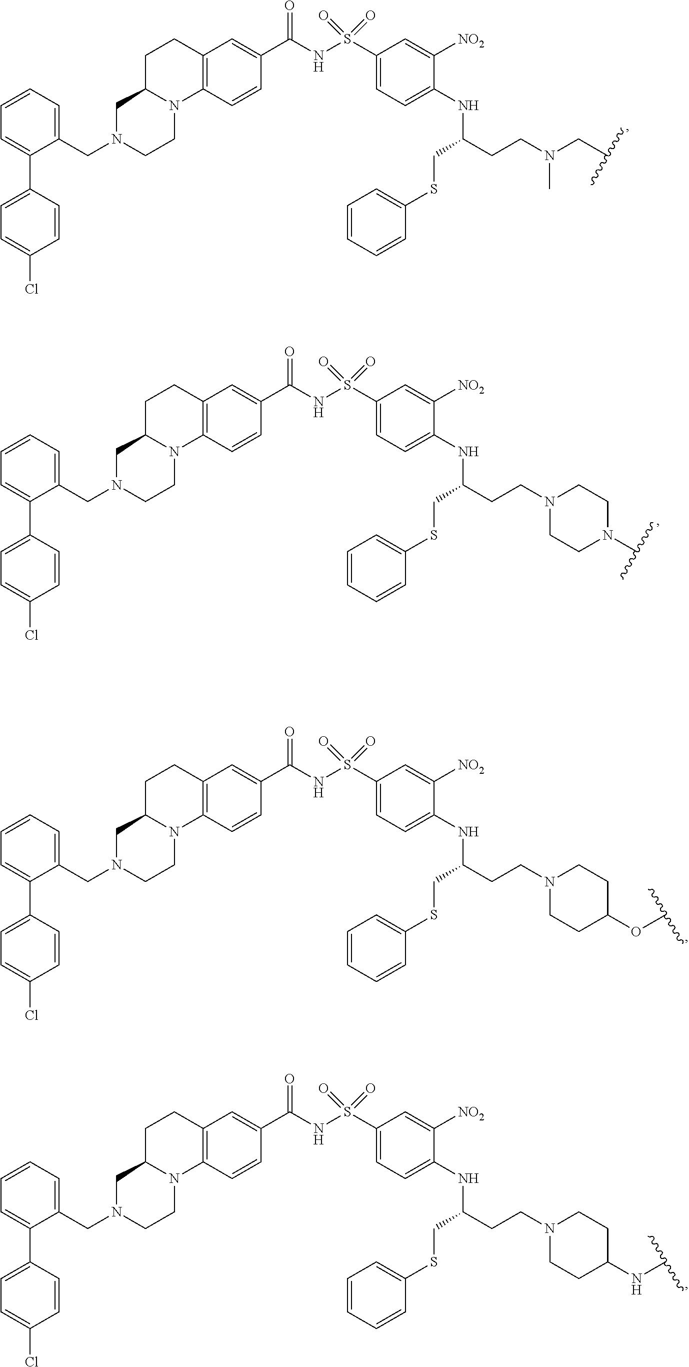 [SMILES: *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1]

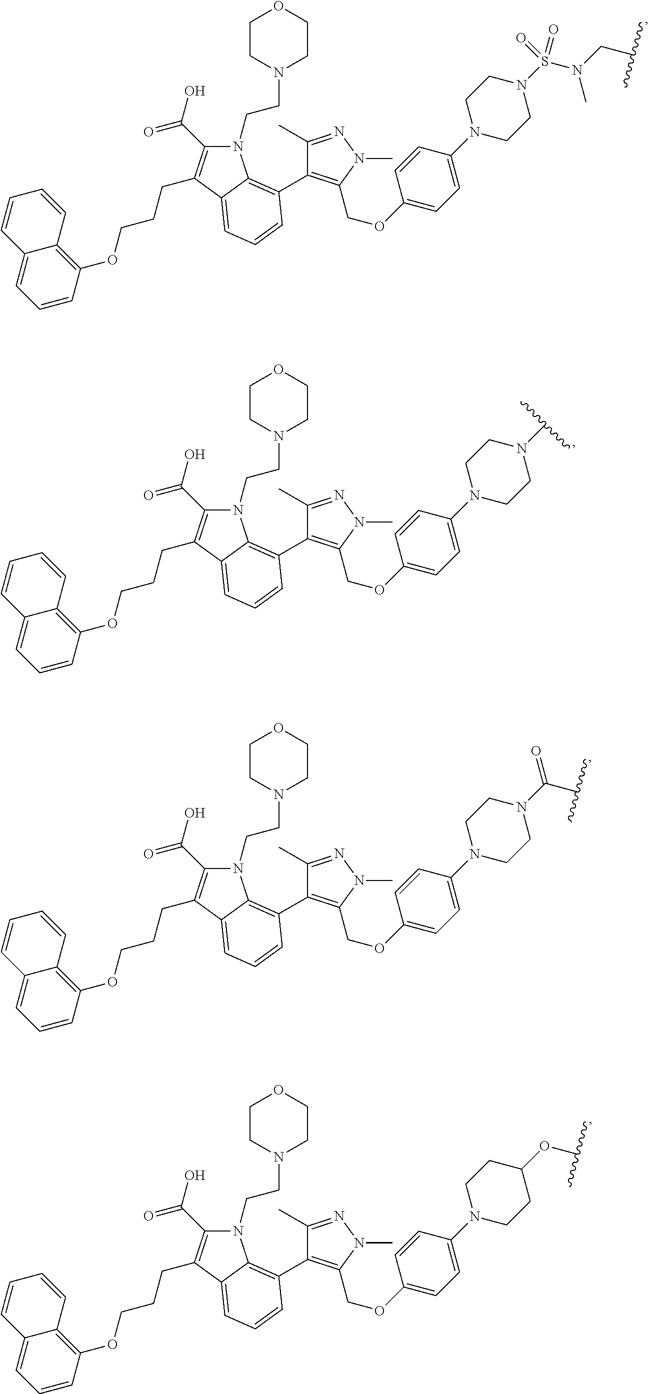 [SMILES: *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *OC1CCN(c2ccc(OCc3c(-c4cccc5c(CCCOc6cccc7ccccc67)c(C(=O)O)n(CCN6CCOCC6)c45)c(C)nn3C)cc2)CC1]

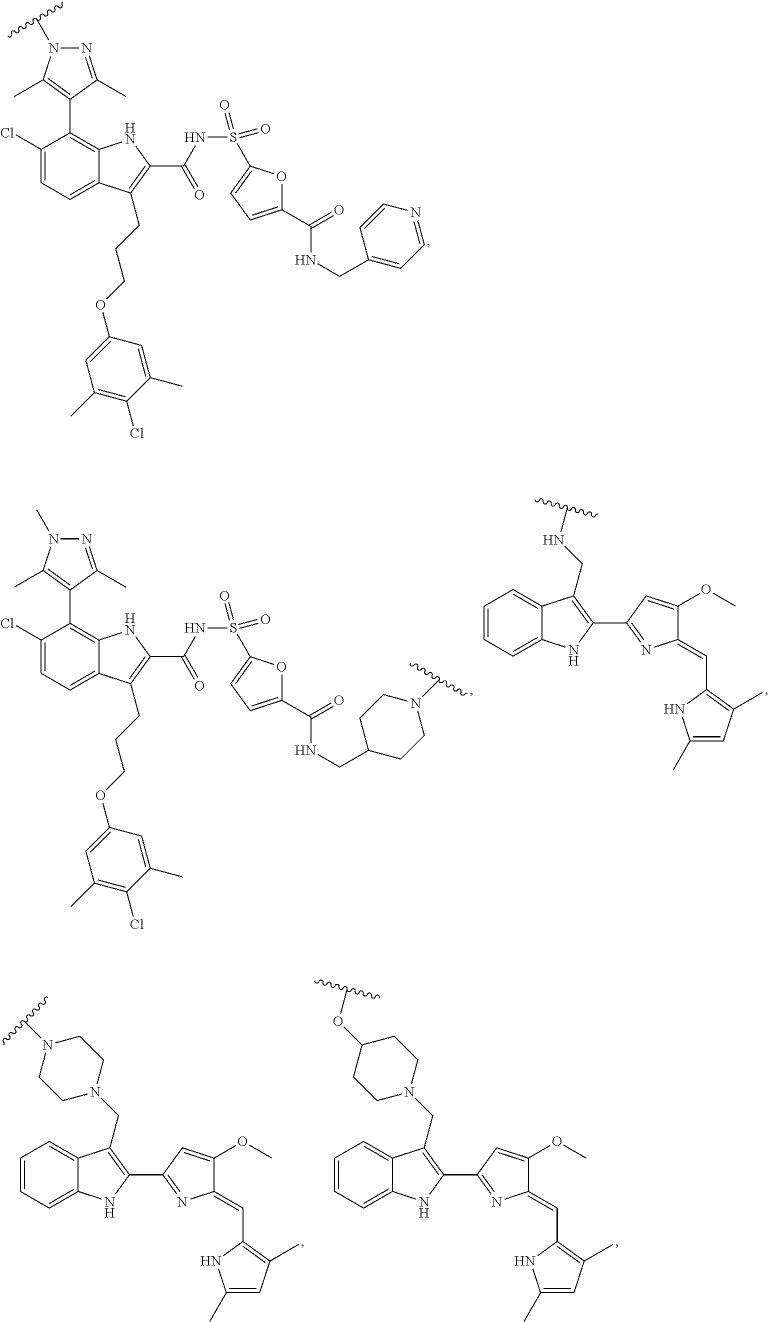 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12 ; *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1]

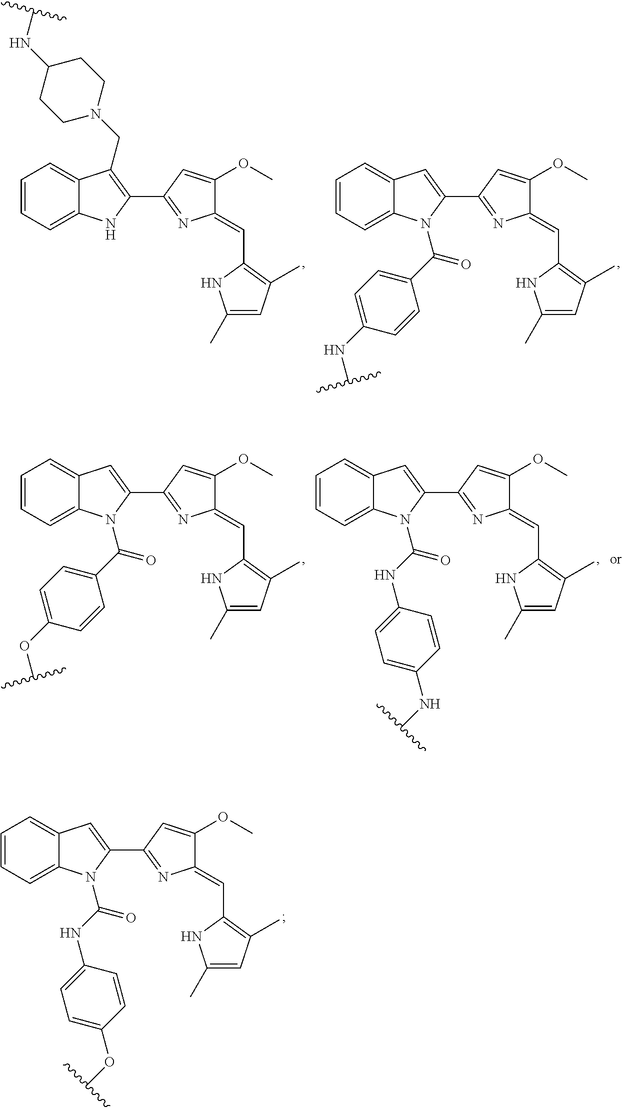

R³ may be absent, an unsubstituted C₁-C₆ alkyl, or a substituted or unsubstituted C₃-C₆ ketone; A may be absent, a bond, or a substituted or unsubstituted C₁-C₆ heterocyclic group; n may be 0 to 3; R⁴ may be a bond or a substituted or unsubstituted C₁-C₁₀ alkyl; and R² may be

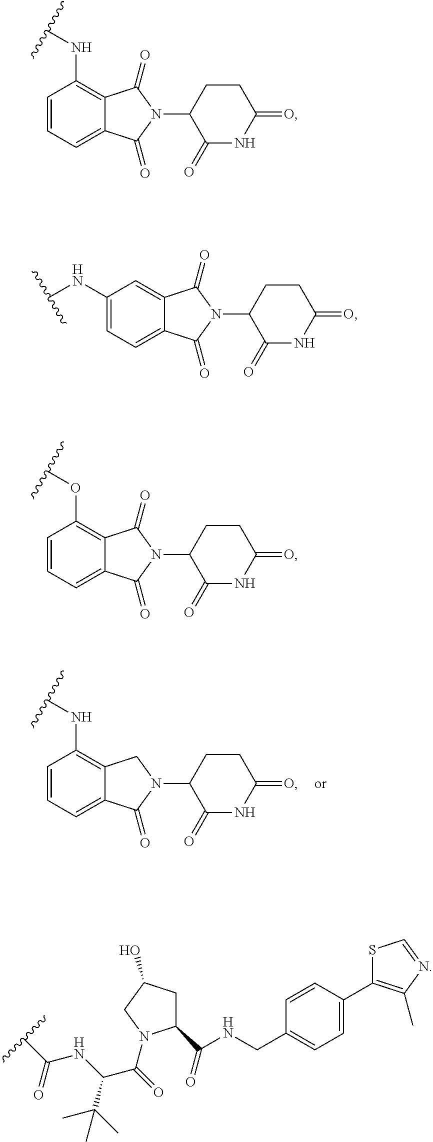 [SMILES: *Nc1cccc2c1C(=O)N(C1CCC(=O)NC1=O)C2=O ; *Nc1ccc2c(c1)C(=O)N(C1CCC(=O)NC1=O)C2=O ; *Oc1cccc2c1C(=O)N(C1CCC(=O)NC1=O)C2=O ; *Nc1cccc2c1CN(C1CCC(=O)NC1=O)C2=O ; *C(=O)N[C@H](C(=O)N1C[C@H](O)C[C@H]1C(=O)NCc1ccc(-c2scnc2C)cc1)C(C)(C)C]

In a preferred embodiment, a compound of the disclosure comprises Formula (II), wherein R¹ may be

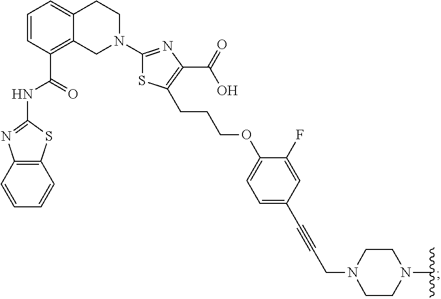 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 2, A may be a triazole; R⁴ may be a bond; and R² may be

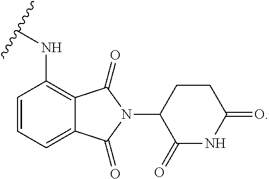 [SMILES: *Nc1cccc2c1C(=O)N(C1CCC(=O)NC1=O)C2=O]

In a preferred embodiment, a compound of the disclosure comprises Formula (II), wherein R¹ may be

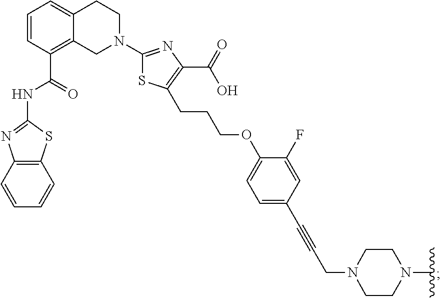 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 1; A may be a triazole; R⁴ may be a bond; and R² may be

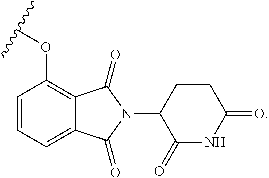 [SMILES: *Oc1cccc2c1C(=O)N(C1CCC(=O)NC1=O)C2=O]

In a preferred embodiment, a compound of the disclosure comprises Formula (II), wherein R¹ may be

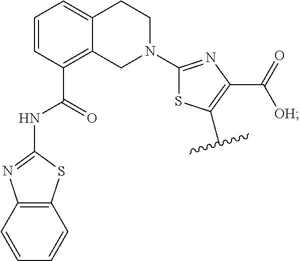 [SMILES: *c1sc(N2CCc3cccc(C(=O)Nc4nc5ccccc5s4)c3C2)nc1C(=O)O]

R³ may be propyl; n may be 2; A may be a triazole; R⁴ may be a bond; and R² may be

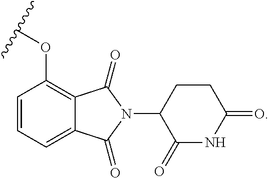 [SMILES: *Oc1cccc2c1C(=O)N(C1CCC(=O)NC1=O)C2=O]

In a preferred embodiment, a compound of the disclosure comprises Formula (II), wherein R¹ may be

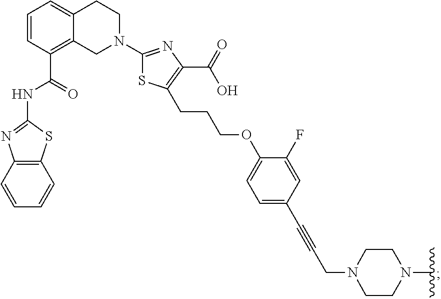 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 3; A may be a triazole; R⁴ may be a bond; and R² may be

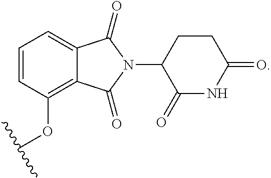 [SMILES: *Oc1cccc2c1C(=O)N(C1CCC(=O)NC1=O)C2=O]

In a preferred embodiment, a compound of the disclosure comprises Formula (II), wherein R¹ may be

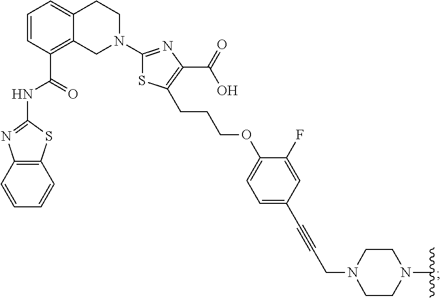 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 1; A may be a triazole; R⁴ may be a bond; and R² may be

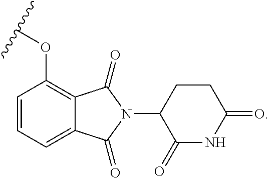 [SMILES: *Oc1cccc2c1C(=O)N(C1CCC(=O)NC1=O)C2=O]

In a preferred embodiment, a compound of the disclosure comprises Formula (II), wherein R¹ may be

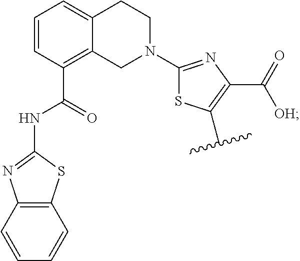 [SMILES: *c1sc(N2CCc3cccc(C(=O)Nc4nc5ccccc5s4)c3C2)nc1C(=O)O]

R³ may be C₃-alkyl; n may be 3; A may be a triazole; R⁴ may be a bond; and R² may be

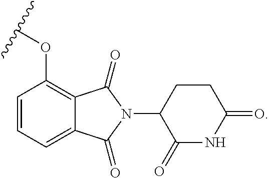 [SMILES: *Oc1cccc2c1C(=O)N(C1CCC(=O)NC1=O)C2=O]

In a preferred embodiment, a compound of the disclosure comprises Formula (II), wherein R¹ may be

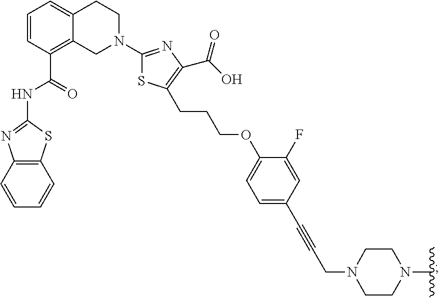 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 2; A may be a triazole; R⁴ may be a bond; and R² may be

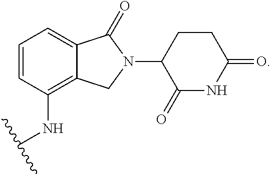 [SMILES: *Nc1cccc2c1CN(C1CCC(=O)NC1=O)C2=O]

In a preferred embodiment, a compound of the disclosure comprises Formula (II), wherein R¹ may be

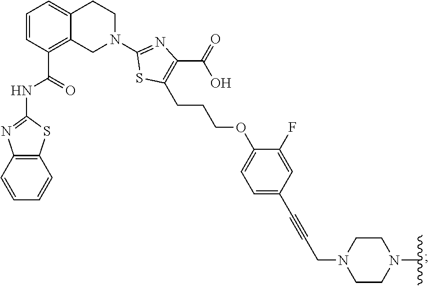 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 1; A may be a triazole; R⁴ may be a bond; and R² may be

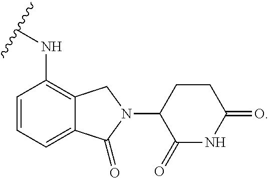 [SMILES: *Nc1cccc2c1CN(C1CCC(=O)NC1=O)C2=O]

In a preferred embodiment, a compound of the disclosure comprises Formula (II), wherein R¹ may be

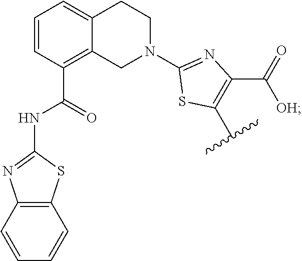 [SMILES: *c1sc(N2CCc3cccc(C(=O)Nc4nc5ccccc5s4)c3C2)nc1C(=O)O]

R³ may be propyl; n may be 2; A may be a triazole; R⁴ may be a bond; and R² may be

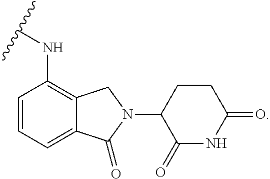 [SMILES: *Nc1cccc2c1CN(C1CCC(=O)NC1=O)C2=O]

In a preferred embodiment, a compound of the disclosure comprises Formula (II), wherein R¹ may be

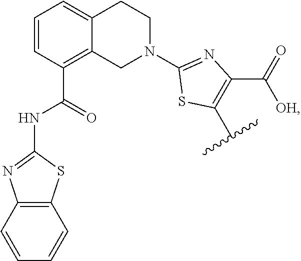 [SMILES: *c1sc(N2CCc3cccc(C(=O)Nc4nc5ccccc5s4)c3C2)nc1C(=O)O]

R³ may be butan-1-amine; A may be absent; n may be 2; R⁴ may be N-(4-ethylamino)butyl)acetamide; and R² may be

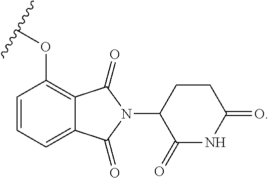 [SMILES: *Oc1cccc2c1C(=O)N(C1CCC(=O)NC1=O)C2=O]

In a preferred embodiment, a compound of the disclosure comprises Formula (II), wherein R¹ may be

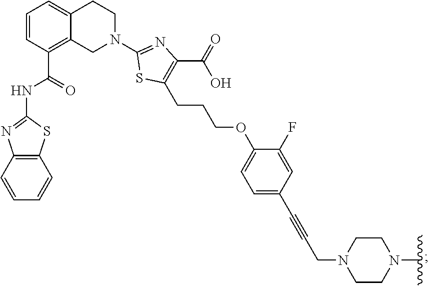 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 2; A may be a triazole; R⁴ may be C(O); and R² may be

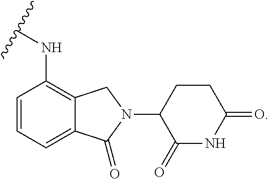 [SMILES: *Nc1cccc2c1CN(C1CCC(=O)NC1=O)C2=O]

In a preferred embodiment, a compound of the disclosure comprises Formula (II), wherein R¹ may be

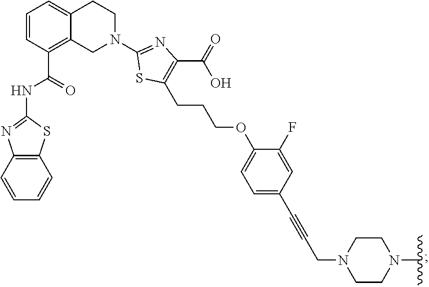 [SMILES: *N1CCN(CC#Cc2ccc(OCCCc3sc(N4CCc5cccc(C(=O)Nc6nc7ccccc7s6)c5C4)nc3C(=O)O)c(F)c2)CC1]

R³ may be 2-pentanone; n may be 2; A may be a triazole; R⁴ may be a bond; and R² may be

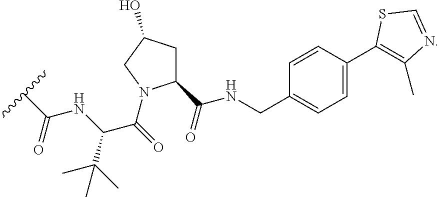 [SMILES: *C(=O)N[C@H](C(=O)N1C[C@H](O)C[C@H]1C(=O)NCc1ccc(-c2scnc2C)cc1)C(C)(C)C]

In a preferred embodiment, a compound of the disclosure comprises Formula (II), wherein R¹ may be

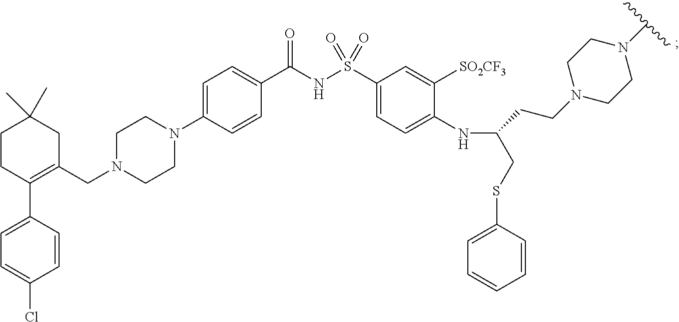

R³ may be 2-pentanone; n may be 2; A may be a triazole; R⁴ may be a bond; and R² may be

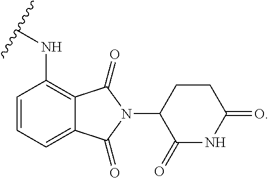 [SMILES: *Nc1cccc2c1C(=O)N(C1CCC(=O)NC1=O)C2=O]

In a preferred embodiment, a compound of the disclosure comprises Formula (II), wherein R¹ may be

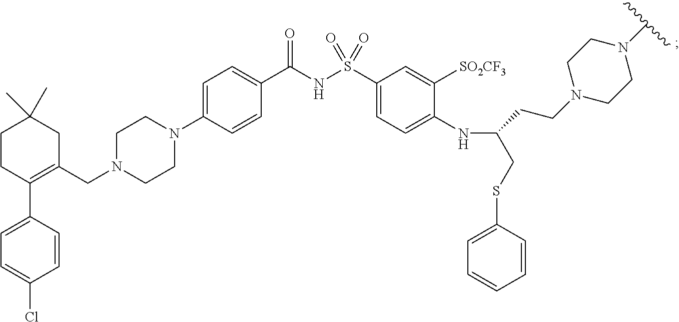

R³ may be 2-pentanone; n may be 1; A may be a triazole; R⁴ may be a bond; and R² may be

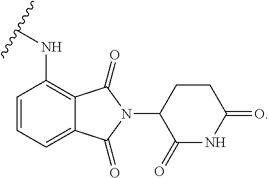 [SMILES: *Nc1cccc2c1C(=O)N(C1CCC(=O)NC1=O)C2=O]

In a preferred embodiment, a compound of the disclosure comprises Formula (II), wherein R¹ may be

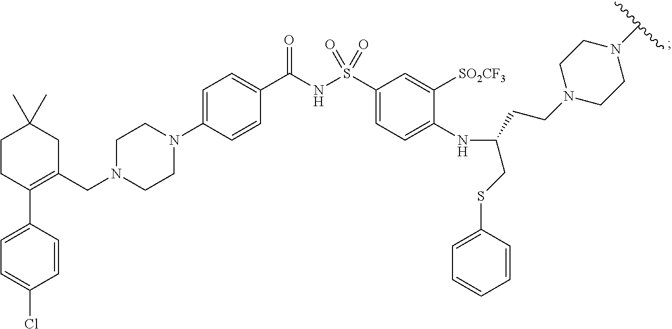

R³ may be C(O)NH; n may be 1; A may be absent; R⁴ may be a bond; and R² may be

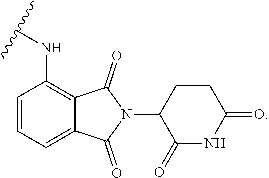 [SMILES: *Nc1cccc2c1C(=O)N(C1CCC(=O)NC1=O)C2=O]

In a preferred embodiment, a compound of the disclosure comprises Formula (II), wherein R¹ may be

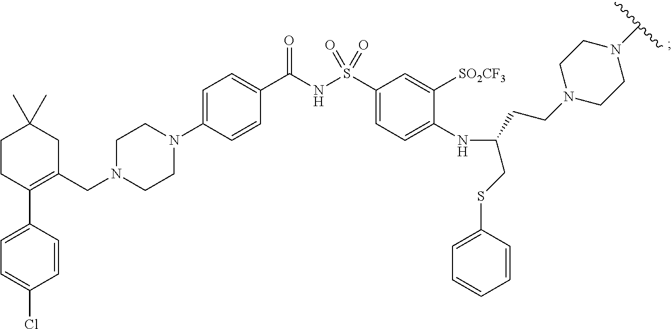

R³ may be C(S)NH; n may be 1; A may be absent; R⁴ may be a bond; and R² may be

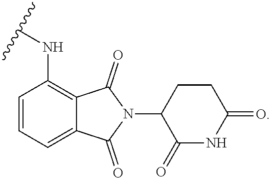 [SMILES: *Nc1cccc2c1C(=O)N(C1CCC(=O)NC1=O)C2=O]

In a preferred embodiment, a compound of the disclosure comprises Formula (II), wherein R¹ may be

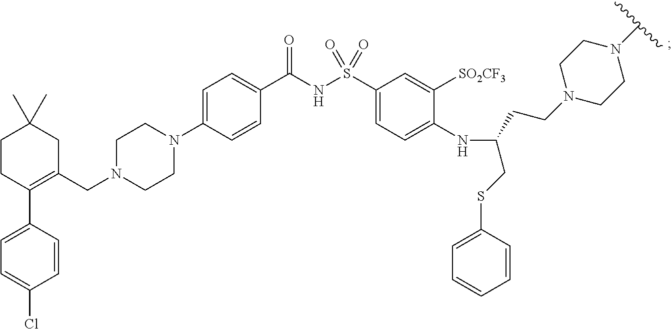

R³ may be C(O); n may be 1; A may be absent; R⁴ may be a bond; and R² may be

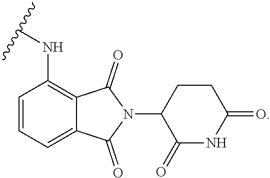 [SMILES: *Nc1cccc2c1C(=O)N(C1CCC(=O)NC1=O)C2=O]

In a preferred embodiment, a compound of the disclosure comprises Formula (II), wherein R¹ may be

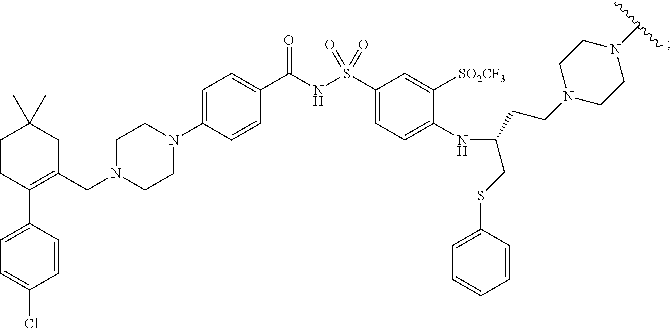

R³ may be C(O); n may be 2; A may be absent; R⁴ may be a bond; and R² may be

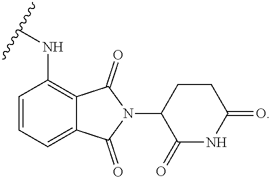 [SMILES: *Nc1cccc2c1C(=O)N(C1CCC(=O)NC1=O)C2=O]

In a preferred embodiment, a compound of the disclosure comprises Formula (II), wherein R¹ may be

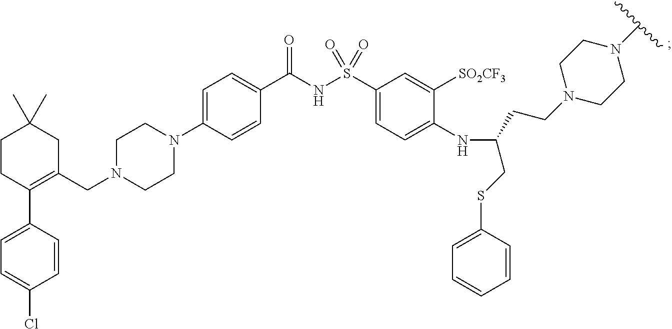

R³ may be C(O); n may be 3; A may be absent; R⁴ may be a bond; and R² may be

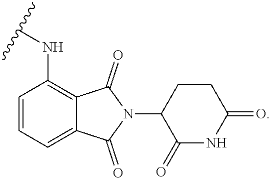 [SMILES: *Nc1cccc2c1C(=O)N(C1CCC(=O)NC1=O)C2=O]

In a preferred embodiment, a compound of the disclosure comprises Formula (II), wherein R¹ may be

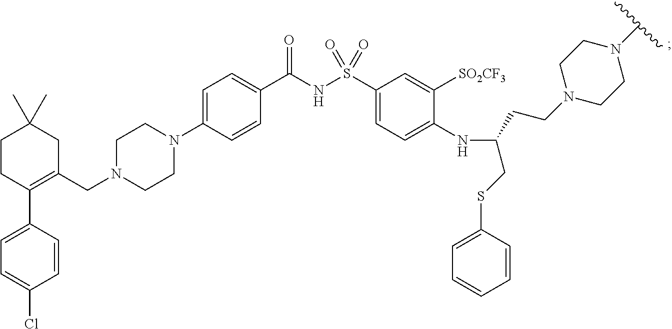

R³ may be C(O); n may be 0; A may be absent; R⁴ may be a bond; and R² may be

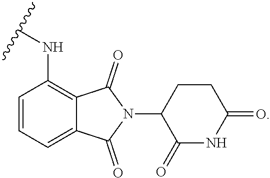 [SMILES: *Nc1cccc2c1C(=O)N(C1CCC(=O)NC1=O)C2=O]

In a preferred embodiment, a compound of the disclosure comprises Formula (II), wherein R¹ may be

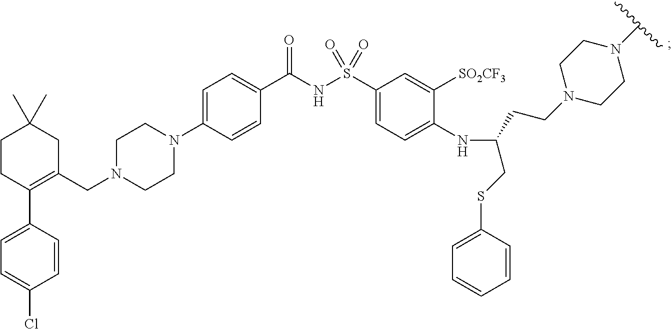

R³ may be a bond; n may be 1; A may be absent; R⁴ may be a bond; and R² may be

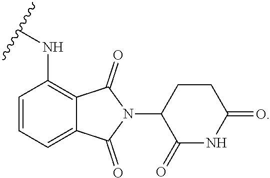 [SMILES: *Nc1cccc2c1C(=O)N(C1CCC(=O)NC1=O)C2=O]

In a preferred embodiment, a compound of the disclosure comprises Formula (II), wherein R¹ may be

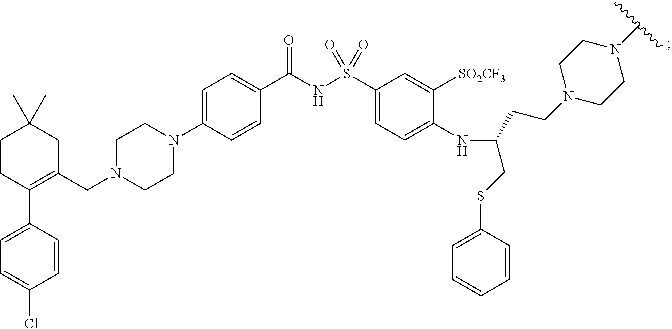

R³ may be C(O)CH₂; n may be 1; A may be a triazole; R⁴ may be a bond; and R² may be

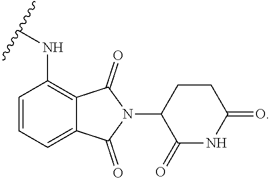 [SMILES: *Nc1cccc2c1C(=O)N(C1CCC(=O)NC1=O)C2=O]

In a preferred embodiment, a compound of the disclosure comprises Formula (II), wherein R¹ may be

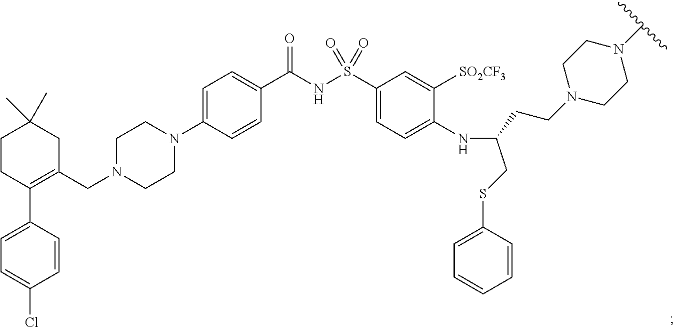

R³ may be C(O)NH; n may be 1; A may be a bond; R⁴ may be (CH₂)₂C(O)NH; and R² may be

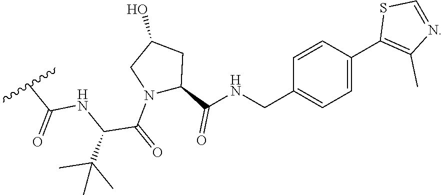 [SMILES: *C(=O)N[C@H](C(=O)N1C[C@H](O)C[C@H]1C(=O)NCc1ccc(-c2scnc2C)cc1)C(C)(C)C]

In a preferred embodiment, a compound of the disclosure comprises Formula (II), wherein R¹ may be

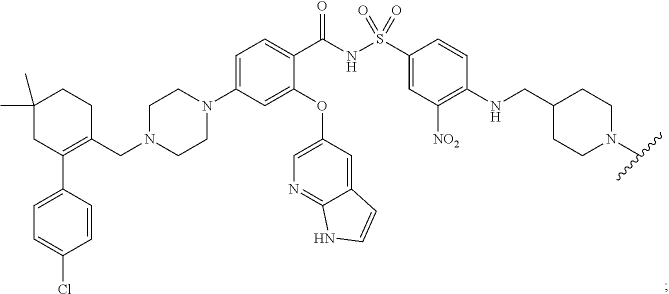 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be C(O); n may be 2; A may be absent; R⁴ may be a bond; and R² may be

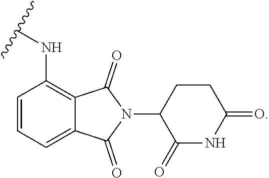 [SMILES: *Nc1cccc2c1C(=O)N(C1CCC(=O)NC1=O)C2=O]

In a preferred embodiment, a compound of the disclosure comprises Formula (II), wherein R¹ may be

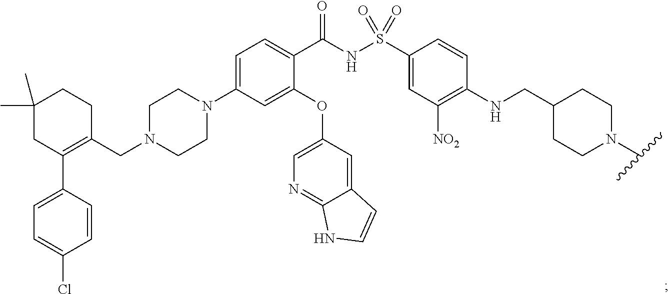 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be C(O)NH; n may be 1; A may be a bond; R⁴ may be (CH₂)₂C(O)NH; and R² may be

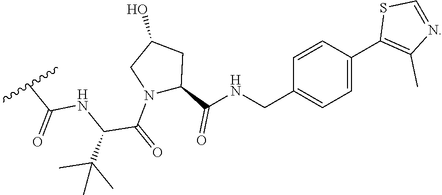 [SMILES: *C(=O)N[C@H](C(=O)N1C[C@H](O)C[C@H]1C(=O)NCc1ccc(-c2scnc2C)cc1)C(C)(C)C]

In a preferred embodiment, a compound of the disclosure comprises Formula (II), wherein R¹ may be

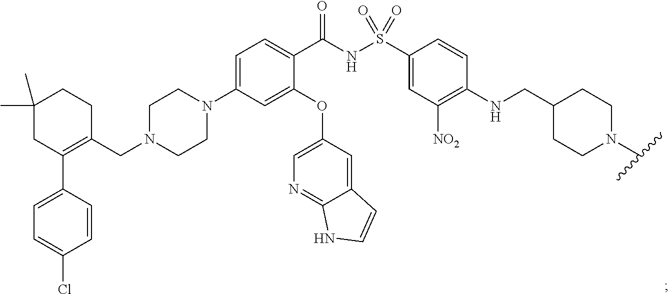 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be a bond; n may be 0; A may be absent; R⁴ may be a bond; and R² may be

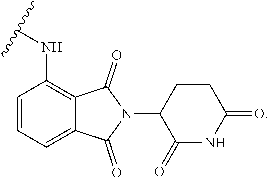 [SMILES: *Nc1cccc2c1C(=O)N(C1CCC(=O)NC1=O)C2=O]

In a preferred embodiment, a compound of the disclosure comprises Formula (II), wherein R¹ may be

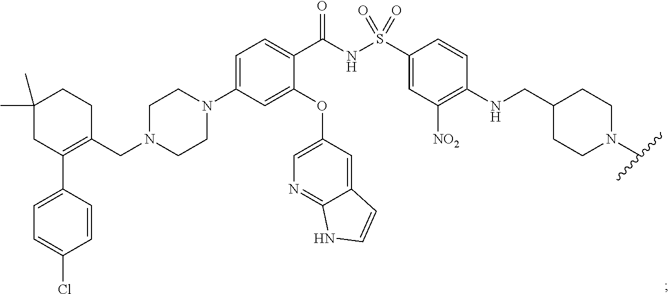 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be a bond; n may be 1; A may be absent; R⁴ may be a bond; and R² may be

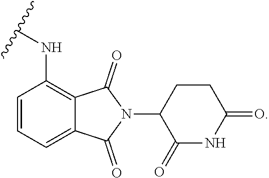 [SMILES: *Nc1cccc2c1C(=O)N(C1CCC(=O)NC1=O)C2=O]

In a preferred embodiment, a compound of the disclosure comprises Formula (II), wherein R¹ may be

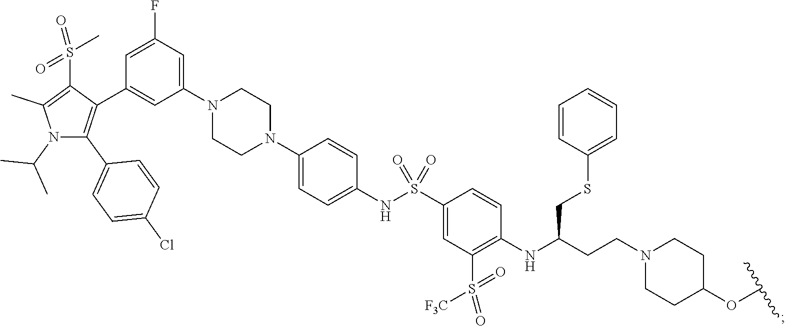 [SMILES: *OC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

R³ may be C(O)NH; n may be 1; A may be absent; R⁴ may be a bond; and R² may be

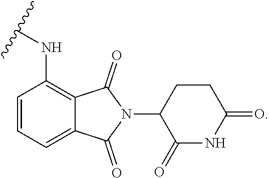 [SMILES: *Nc1cccc2c1C(=O)N(C1CCC(=O)NC1=O)C2=O]

In a preferred embodiment, a compound of the disclosure comprises Formula (II), wherein R¹ may be

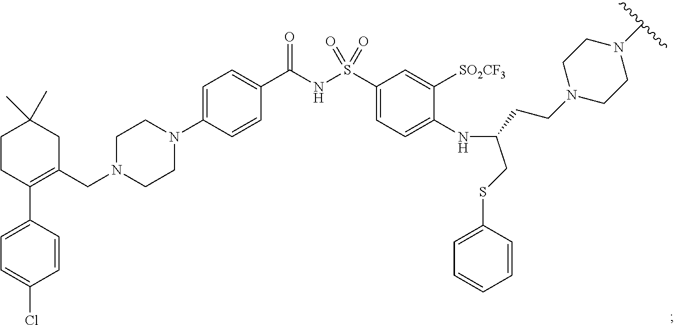

R¹ may be a bond; A may be absent; n may be 2; R⁴ may be N-(4-ethylamino)butyl)acetamide; and R² may be

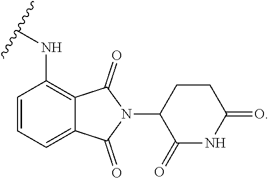 [SMILES: *Nc1cccc2c1C(=O)N(C1CCC(=O)NC1=O)C2=O]

In a preferred embodiment, a compound of the disclosure comprises Formula (II), wherein R¹ may be

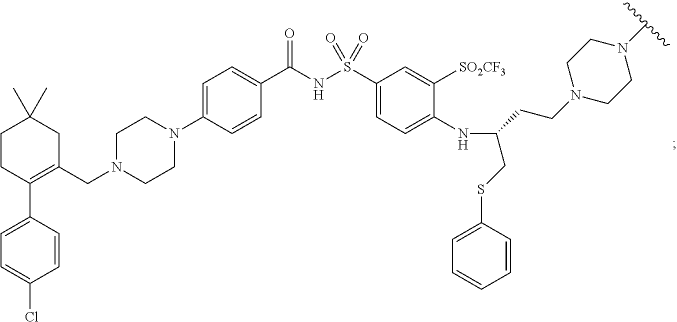

R³ may be a bond; A may be absent; n may be 2; R⁴ may be N-(4-ethylamino)butyl)acetamide; and R² may be

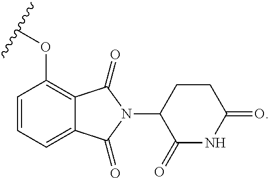 [SMILES: *Oc1cccc2c1C(=O)N(C1CCC(=O)NC1=O)C2=O]

In a preferred embodiment, a compound of the disclosure comprises Formula (II), wherein R¹ may be

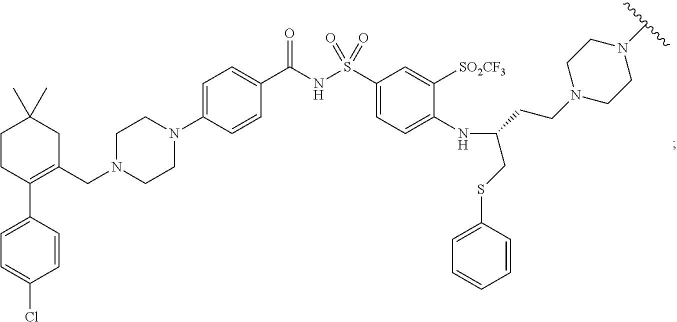

R³ may be a bond; A may be absent; n may be 2; R⁴ may be N-(4-ethylamino)butyl)acetamide; and R² may be

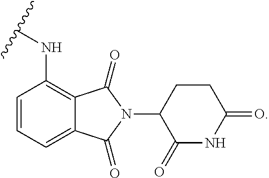 [SMILES: *Nc1cccc2c1C(=O)N(C1CCC(=O)NC1=O)C2=O]

In a preferred embodiment, a compound of the disclosure comprises Formula (II), wherein R¹ may be

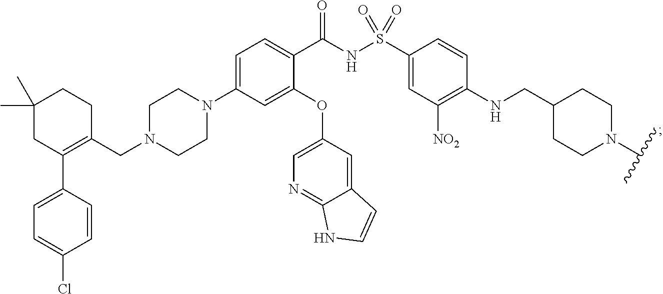 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be a bond; A may be absent; n may be 2; R⁴ may be a bond; and R² may be

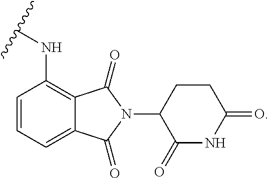 [SMILES: *Nc1cccc2c1C(=O)N(C1CCC(=O)NC1=O)C2=O]

In a preferred embodiment, a compound of the disclosure comprises Formula (II), wherein R¹ may be

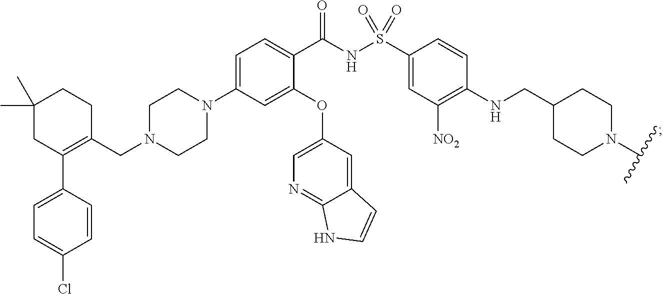 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be 2-pentanone; A may be a triazole; n may be 2; R⁴ may be a bond; and R² may be

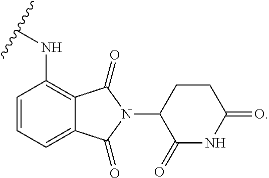 [SMILES: *Nc1cccc2c1C(=O)N(C1CCC(=O)NC1=O)C2=O]

In a preferred embodiment, a compound of the disclosure comprises Formula (II), wherein R¹ may be

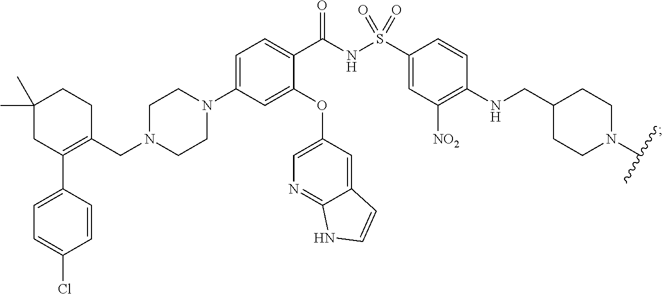 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1]

R³ may be 2-pentanone; A may be a triazole; n may be 2; R⁴ may be a bond; and R² may be

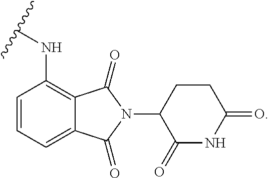 [SMILES: *Nc1cccc2c1C(=O)N(C1CCC(=O)NC1=O)C2=O]

In a preferred embodiment, a compound of the disclosure comprises Formula (II), wherein R¹ may be

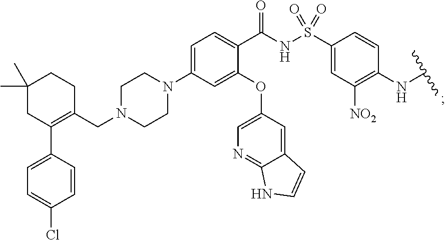 [SMILES: *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

R³ may be N-ethylpropionamide; A may be a triazole; n may be 2; R⁴ may be a bond; and R² may be

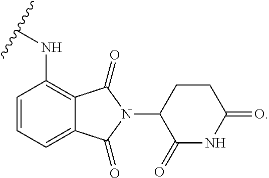 [SMILES: *Nc1cccc2c1C(=O)N(C1CCC(=O)NC1=O)C2=O]

In a preferred embodiment, a compound of the disclosure comprises Formula (II), wherein R¹ may be

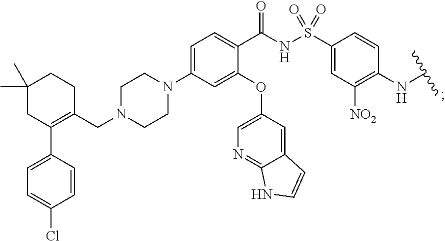 [SMILES: *Nc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]

R³ may be propyl; A may be triazole; n may be 3; R⁴ may be a bond; and R² may be

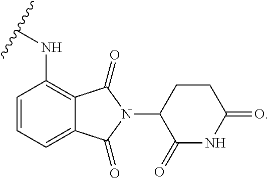 [SMILES: *Nc1cccc2c1C(=O)N(C1CCC(=O)NC1=O)C2=O]

In a preferred embodiment, a compound of the disclosure comprises Formula (II), wherein R¹ may be

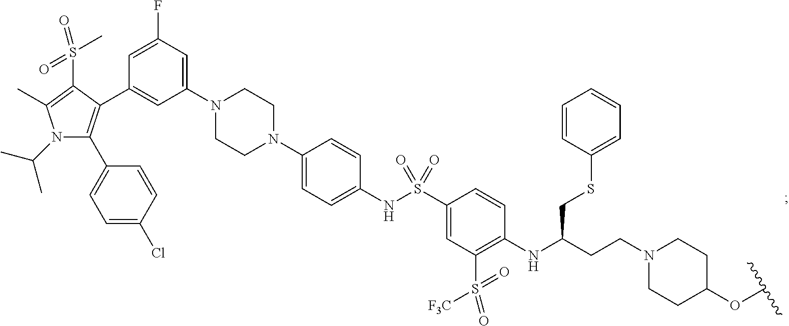 [SMILES: *OC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

R³ may be a bond; A is absent; n may be 3; R⁴ may be N-(4-(ethylamino)butyl)acetamide; and R² may be

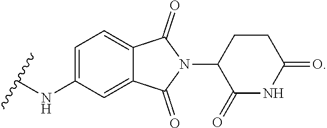 [SMILES: *Nc1ccc2c(c1)C(=O)N(C1CCC(=O)NC1=O)C2=O]

In a preferred embodiment, a compound of the disclosure comprises Formula (II), wherein R¹ may be

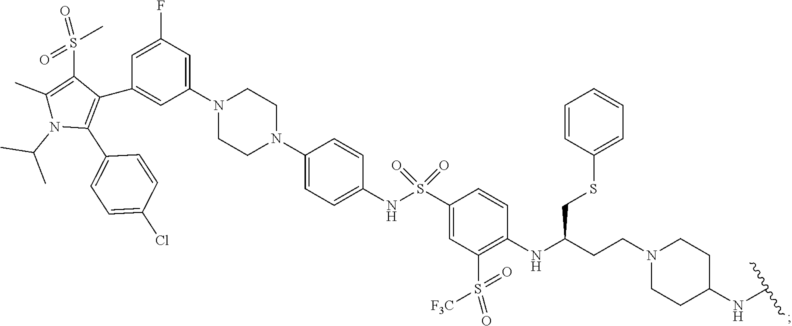 [SMILES: *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

R³ may be propyl; A is a triazole; n may be 2; R⁴ may be a bond; and R² may be

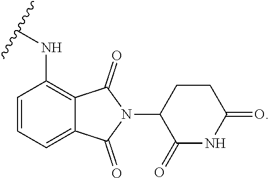 [SMILES: *Nc1cccc2c1C(=O)N(C1CCC(=O)NC1=O)C2=O]

In a preferred embodiment, a compound of the disclosure comprises Formula (II), wherein R¹ may be

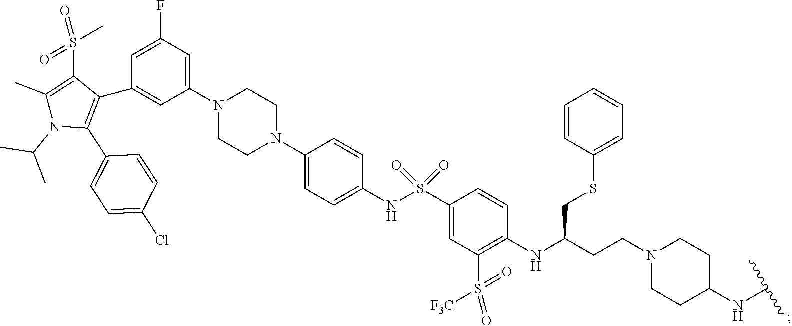 [SMILES: *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

R³ may be a bond; A is absent; n may be 2; R⁴ may be N-(4-(ethylamino)butyl)acetamide; and R² may be

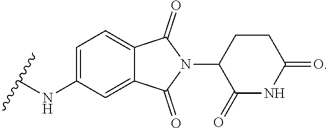 [SMILES: *Nc1ccc2c(c1)C(=O)N(C1CCC(=O)NC1=O)C2=O]

In a preferred embodiment, a compound of the disclosure comprises Formula (II), wherein R¹ may be

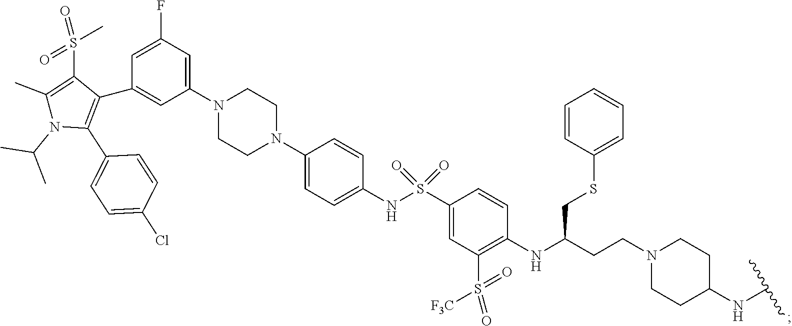 [SMILES: *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

R³ may be a propyl; A is a triazole; n may be 2; R⁴ may be a bond; and R² may be

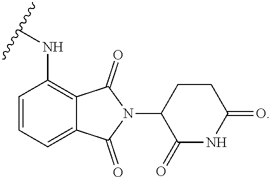 [SMILES: *Nc1cccc2c1C(=O)N(C1CCC(=O)NC1=O)C2=O]

In a preferred embodiment, a compound of the disclosure comprises Formula (II), wherein R¹ may be

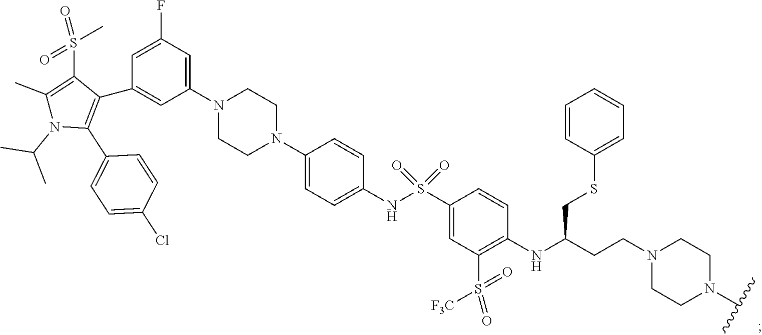 [SMILES: *N1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

R³ may by 2-pentanone; A may be triazole; n may be 2; R⁴ may be a bond; and R² may be

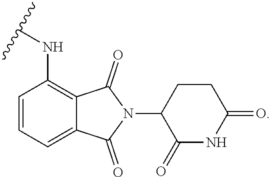 [SMILES: *Nc1cccc2c1C(=O)N(C1CCC(=O)NC1=O)C2=O]

In a preferred embodiment, a compound of the disclosure comprises Formula (II), wherein R¹ may be

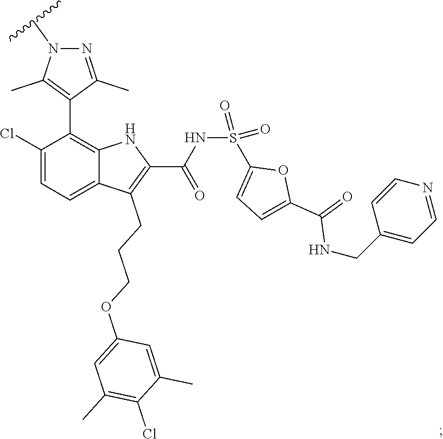 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C]

R³ may by N-methylacetamide; A may be a triazole; n may be 2; R⁴ may be a bond; and R² may be

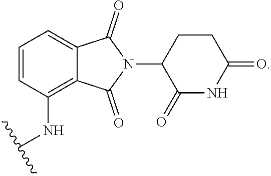 [SMILES: *Nc1cccc2c1C(=O)N(C1CCC(=O)NC1=O)C2=O]

In a preferred embodiment, a compound of the disclosure comprises Formula (II), wherein R¹ may be

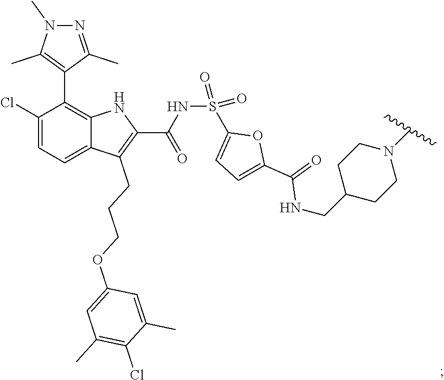 [SMILES: *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1]

R³ may by 2-pentanone; A may be a triazole; n may be 1; R⁴ may be a bond; and R² may be

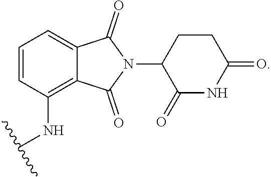 [SMILES: *Nc1cccc2c1C(=O)N(C1CCC(=O)NC1=O)C2=O]

In an exemplary embodiment, a compound of Formula (II) comprises any of the preceding compounds of Formula (II), may be selected from the group consisting of:

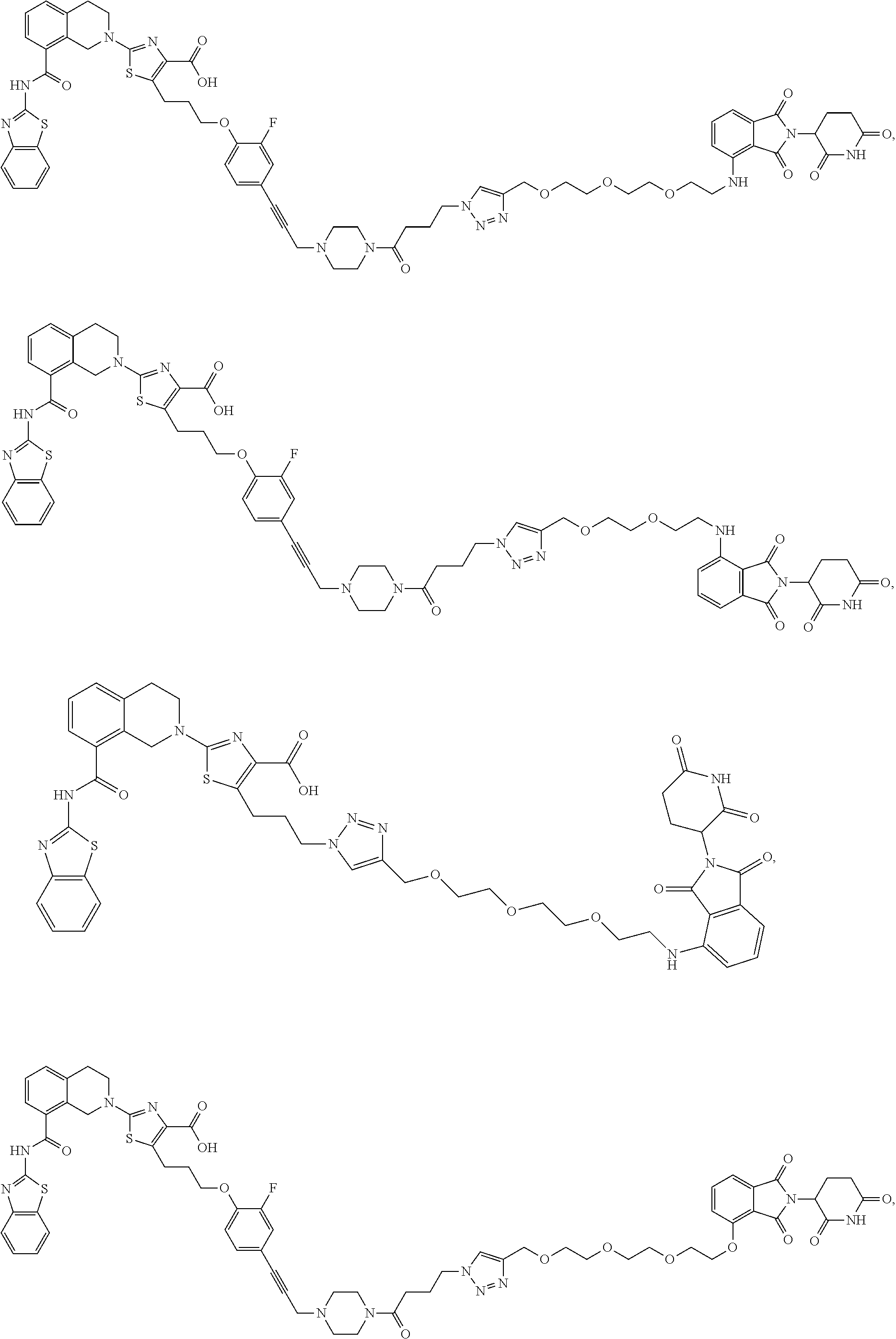

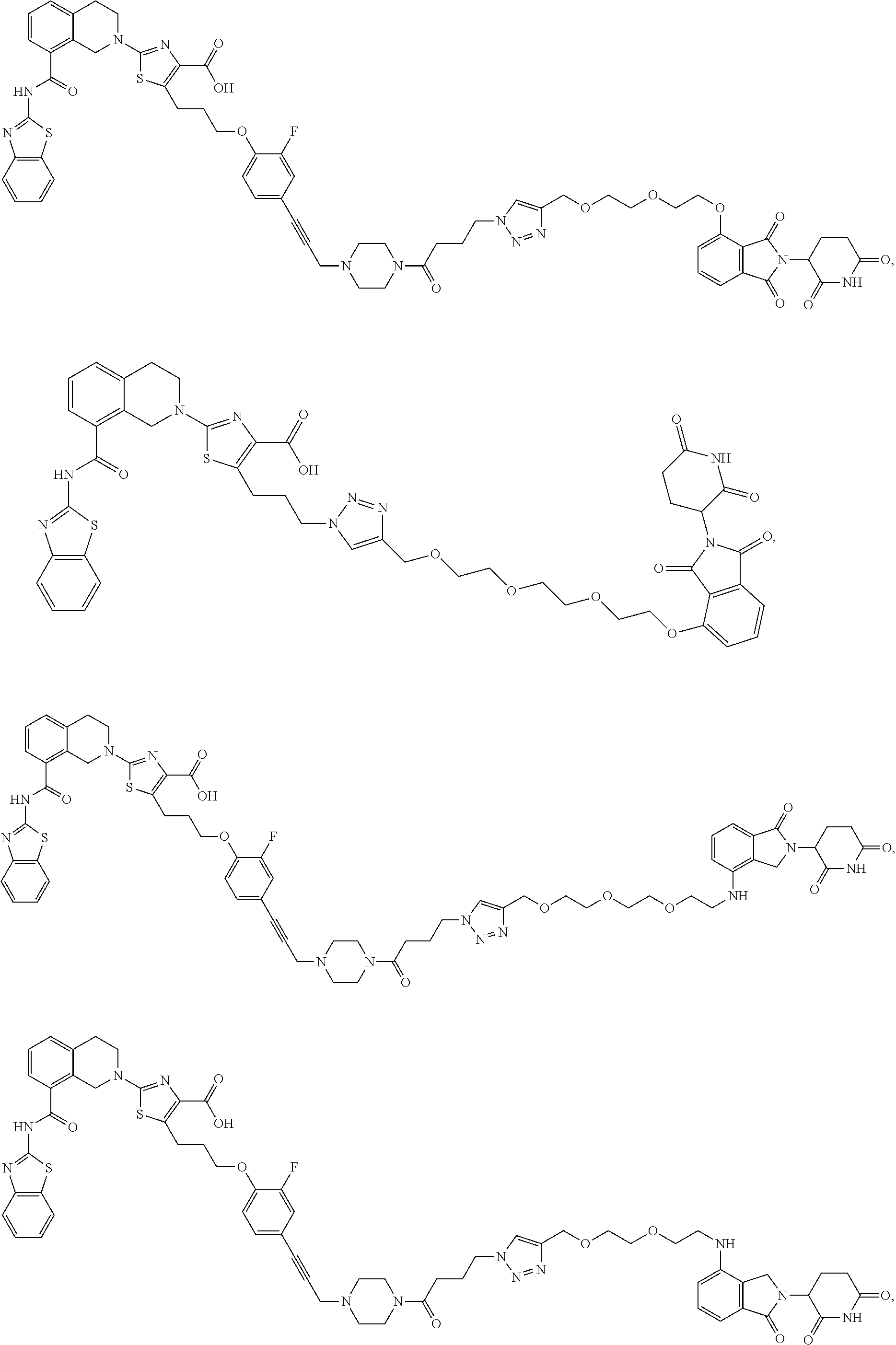

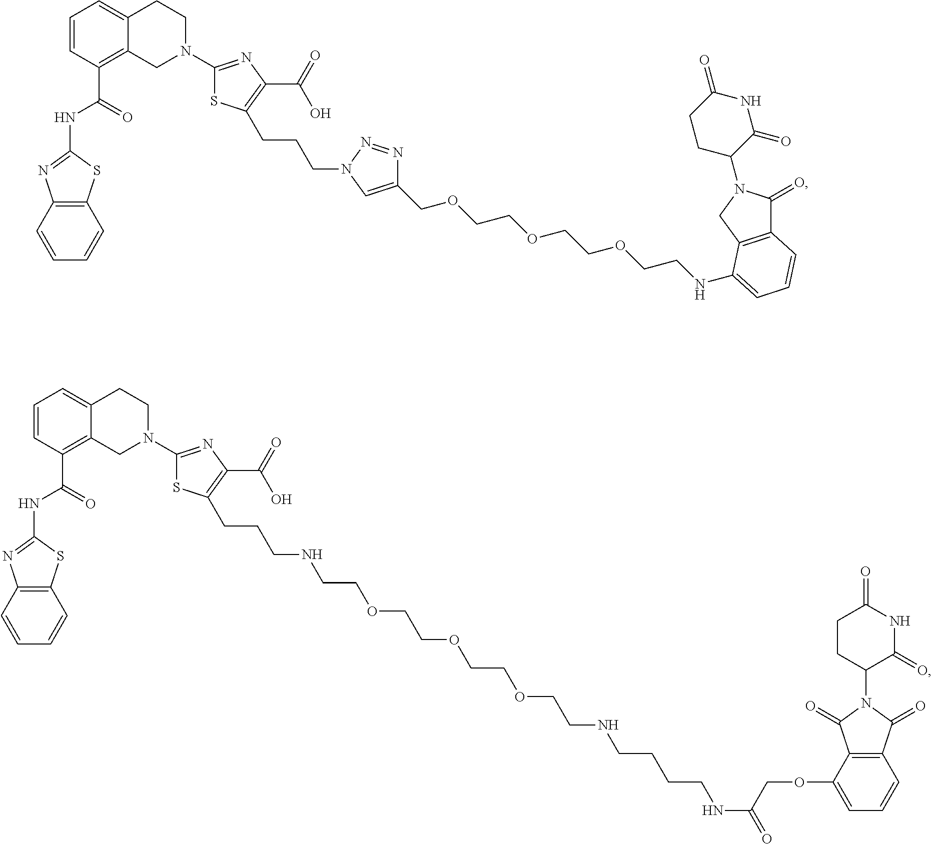 [SMILES: O=C1CCC(N2Cc3c(NCCOCCOCCOCc4cn(CCCc5sc(N6CCc7cccc(C(=O)Nc8nc9ccccc9s8)c7C6)nc5C(=O)O)nn4)cccc3C2=O)C(=O)N1 ; O=C(COc1cccc2c1C(=O)N(C1CCC(=O)NC1=O)C2=O)NCCCCNCCOCCOCCOCCNCCCc1sc(N2CCc3cccc(C(=O)Nc4nc5ccccc5s4)c3C2)nc1C(=O)O]

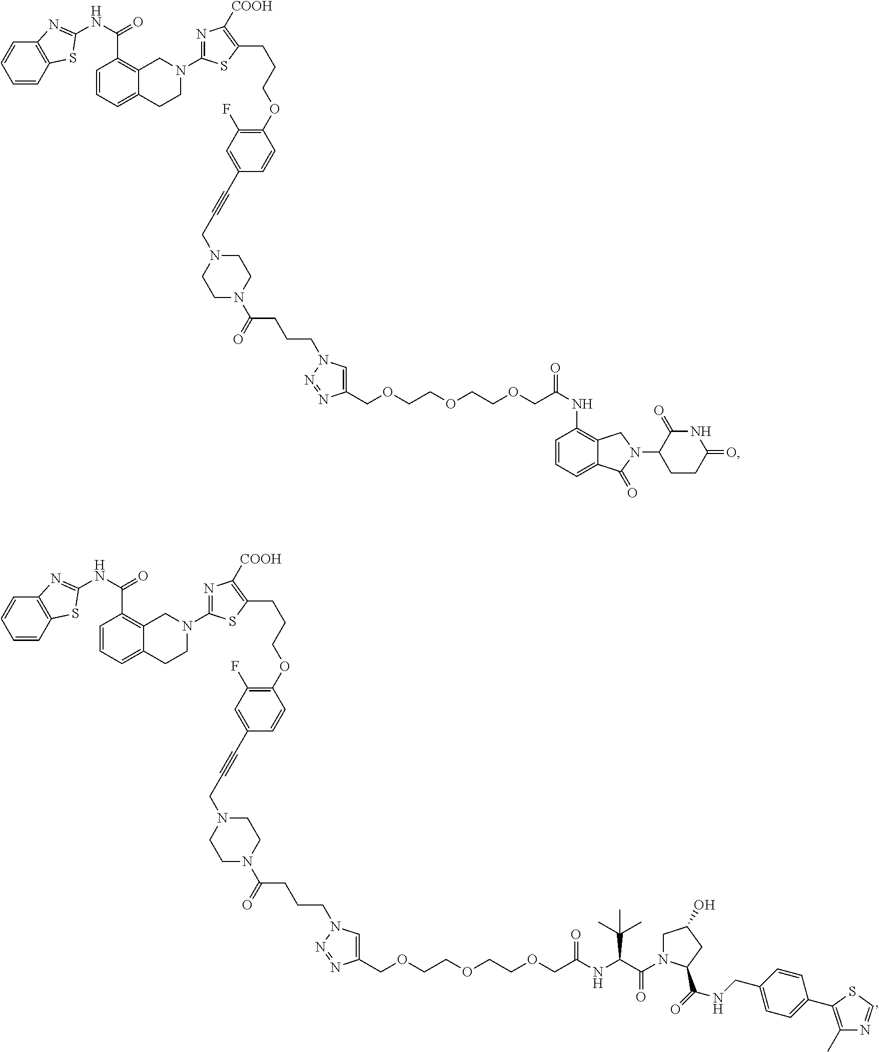 [SMILES: O=C1CCC(N2Cc3c(NC(=O)COCCOCCOCc4cn(CCCC(=O)N5CCN(CC#Cc6ccc(OCCCc7sc(N8CCc9cccc(C(=O)Nc%10nc%11ccccc%11s%10)c9C8)nc7C(=O)O)c(F)c6)CC5)nn4)cccc3C2=O)C(=O)N1 ; Cc1ncsc1-c1ccc(CNC(=O)[C@@H]2C[C@@H](O)CN2C(=O)[C@@H](NC(=O)COCCOCCOCc2cn(CCCC(=O)N3CCN(CC#Cc4ccc(OCCCc5sc(N6CCc7cccc(C(=O)Nc8nc9ccccc9s8)c7C6)nc5C(=O)O)c(F)c4)CC3)nn2)C(C)(C)C)cc1]

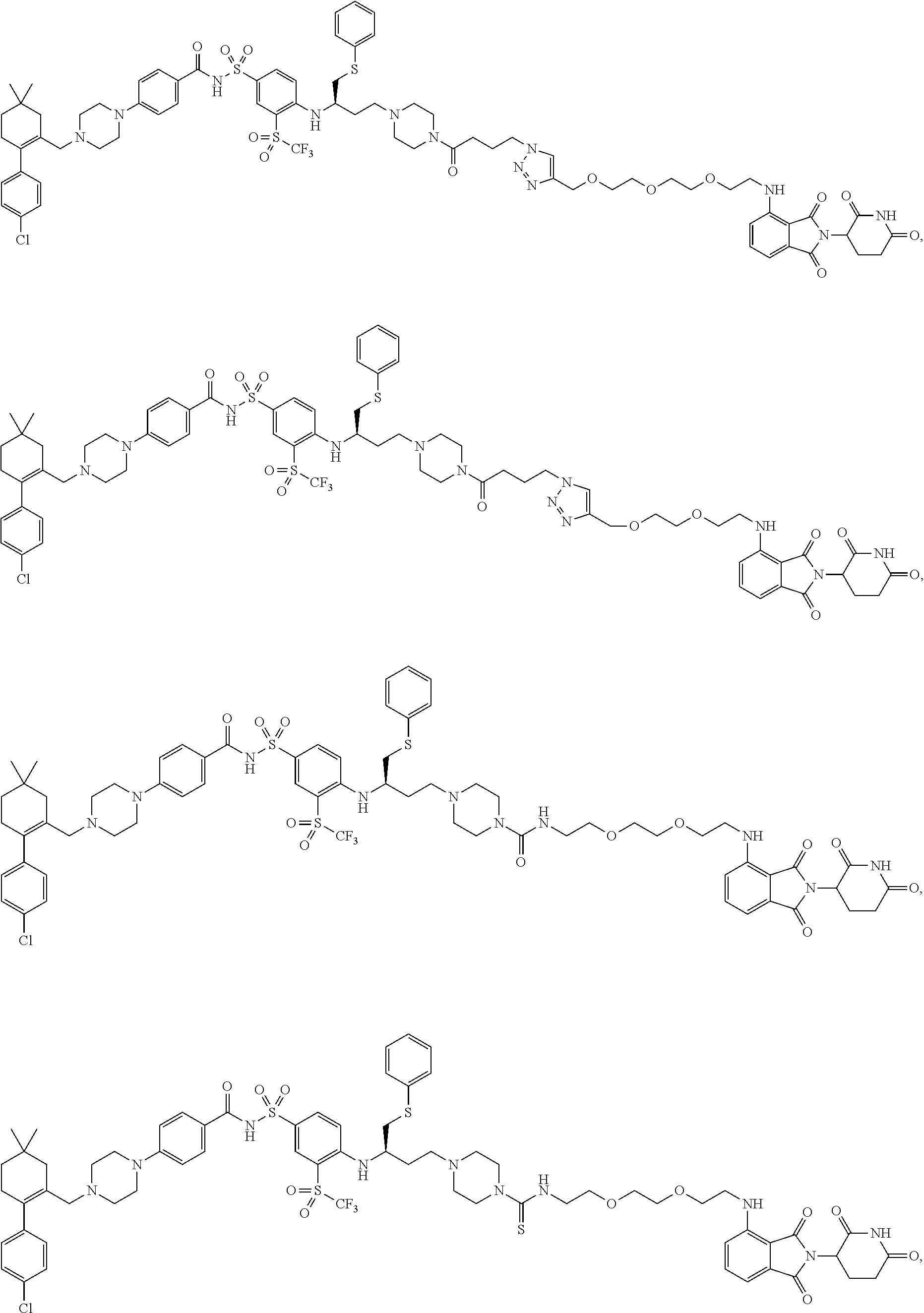

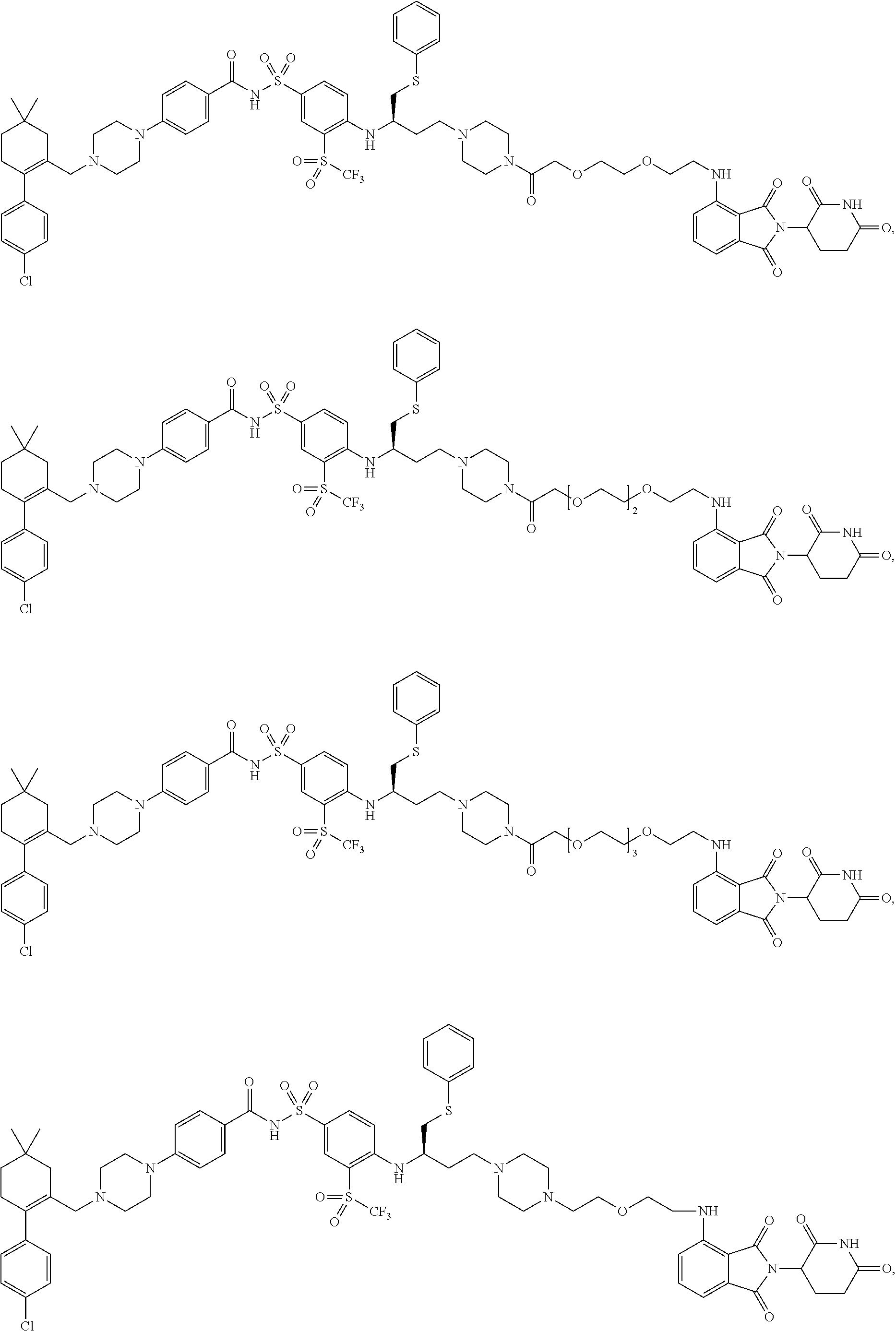

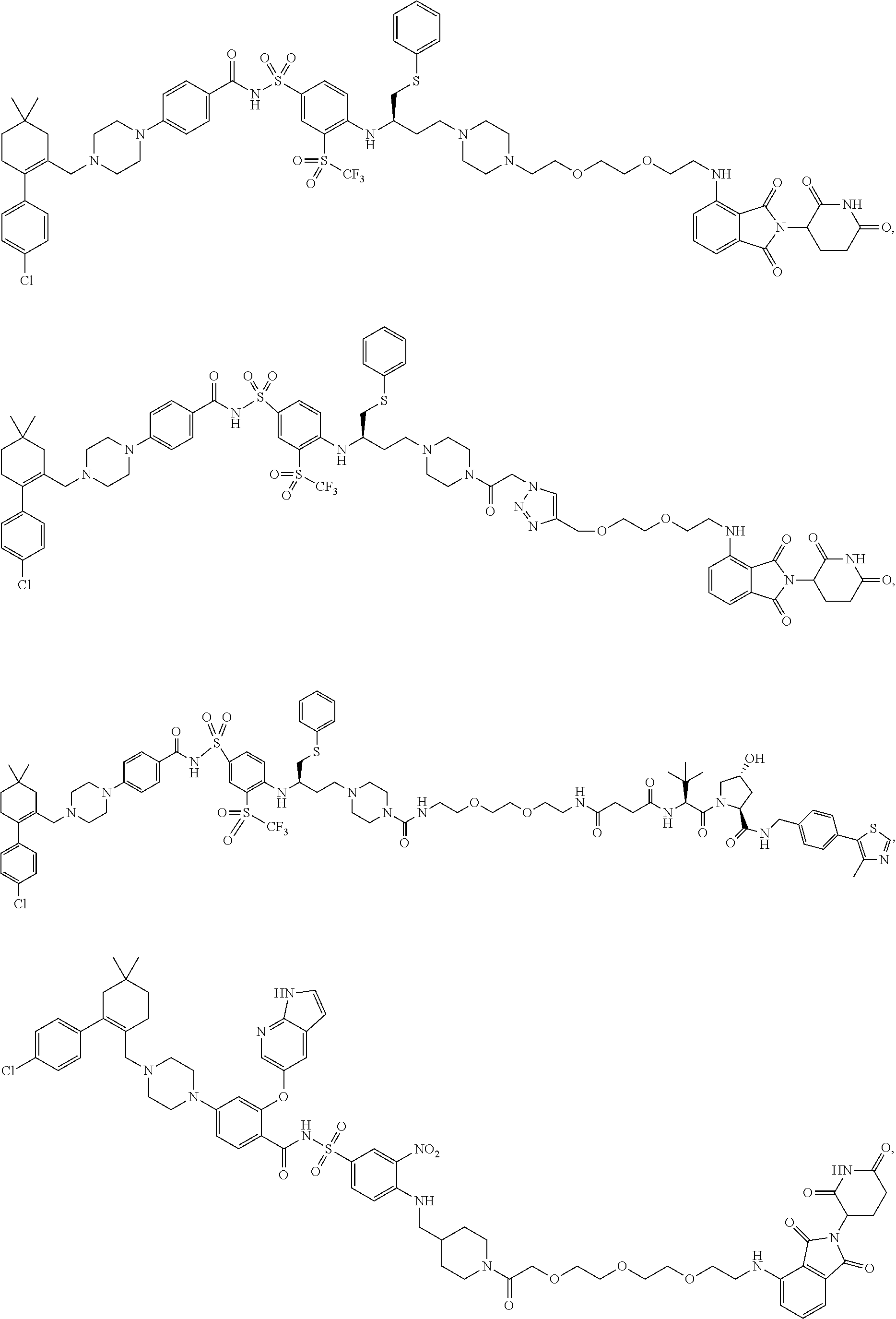

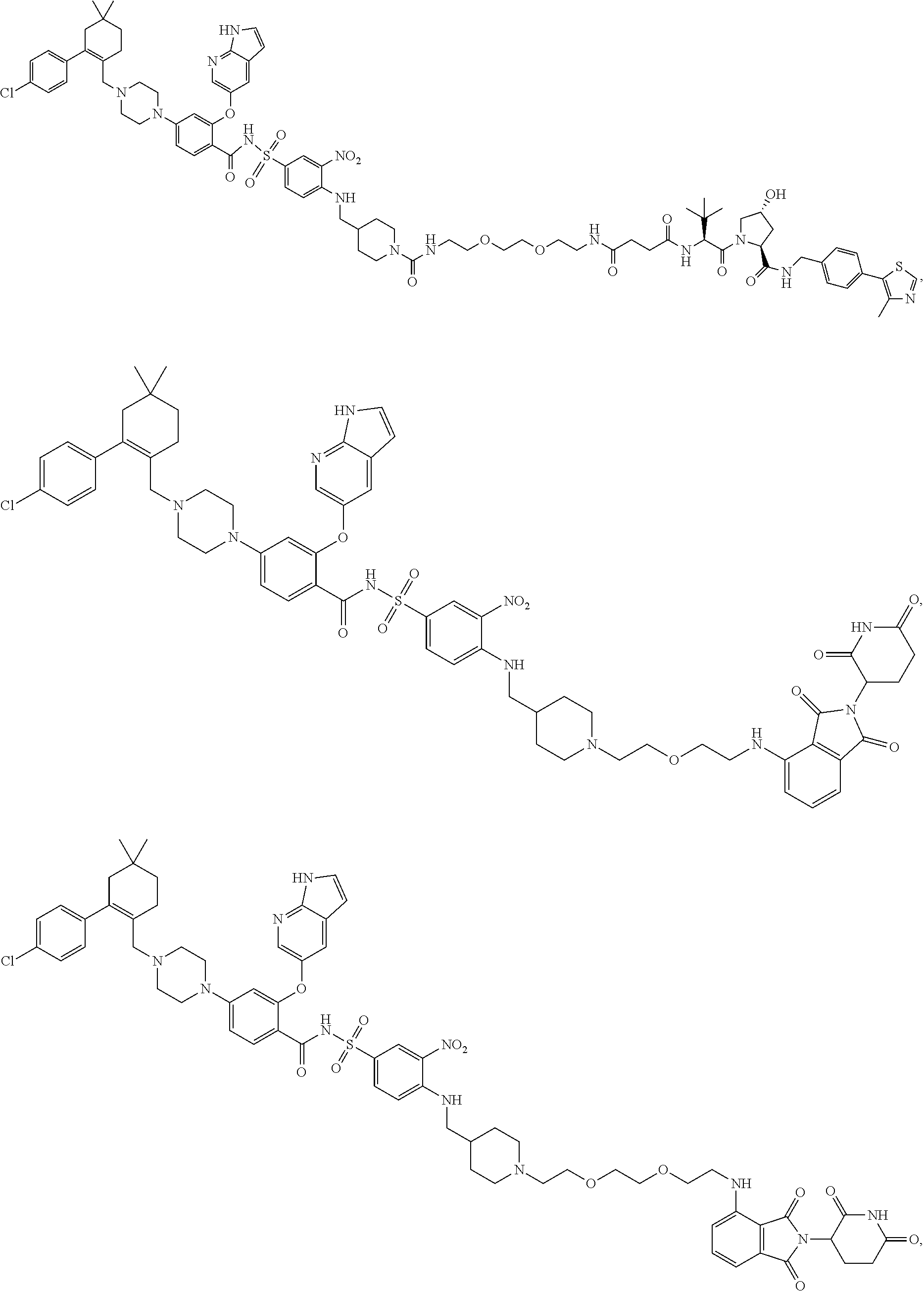

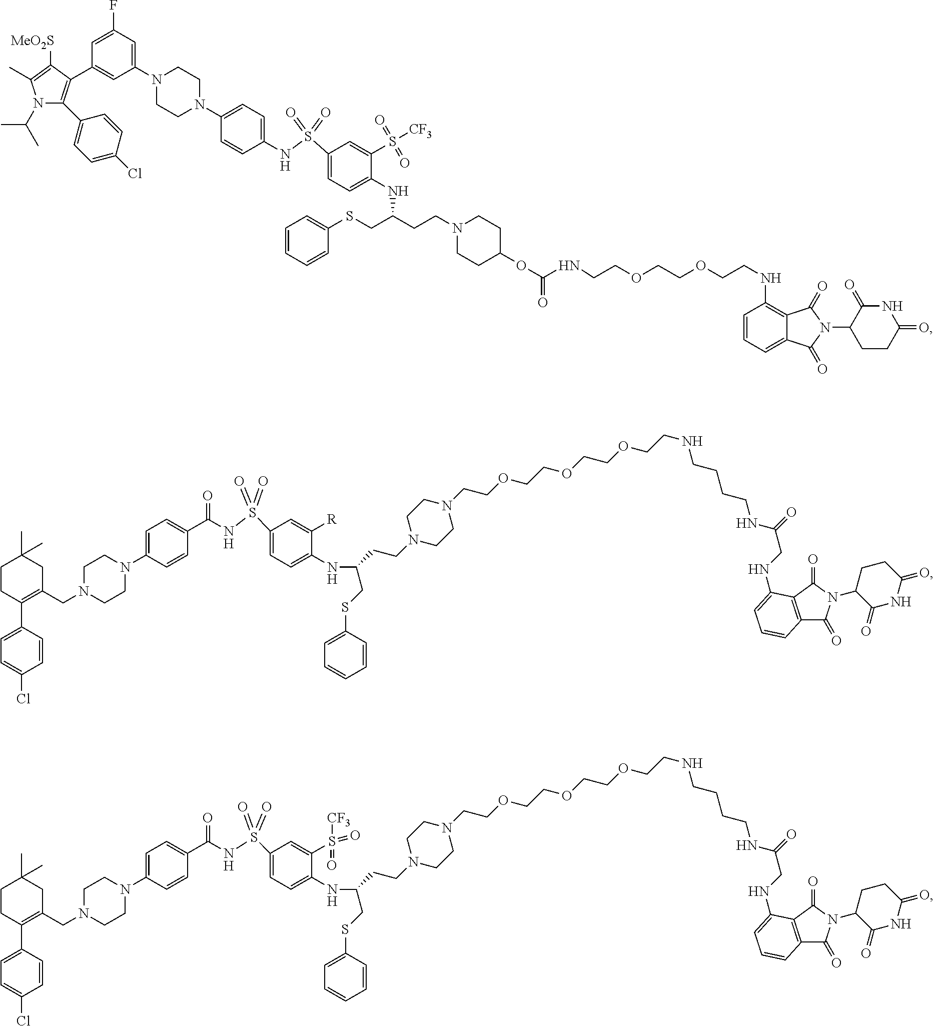

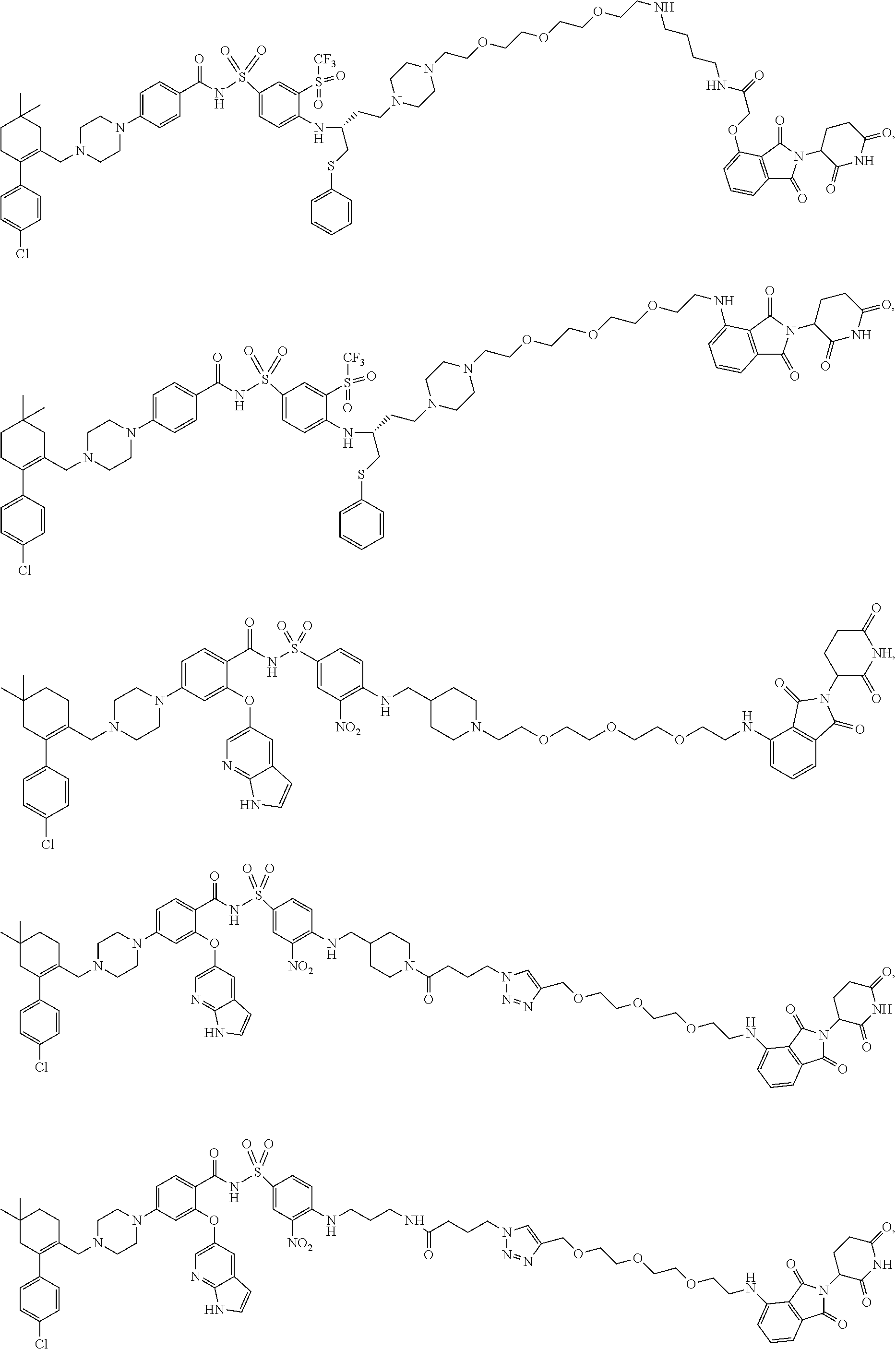

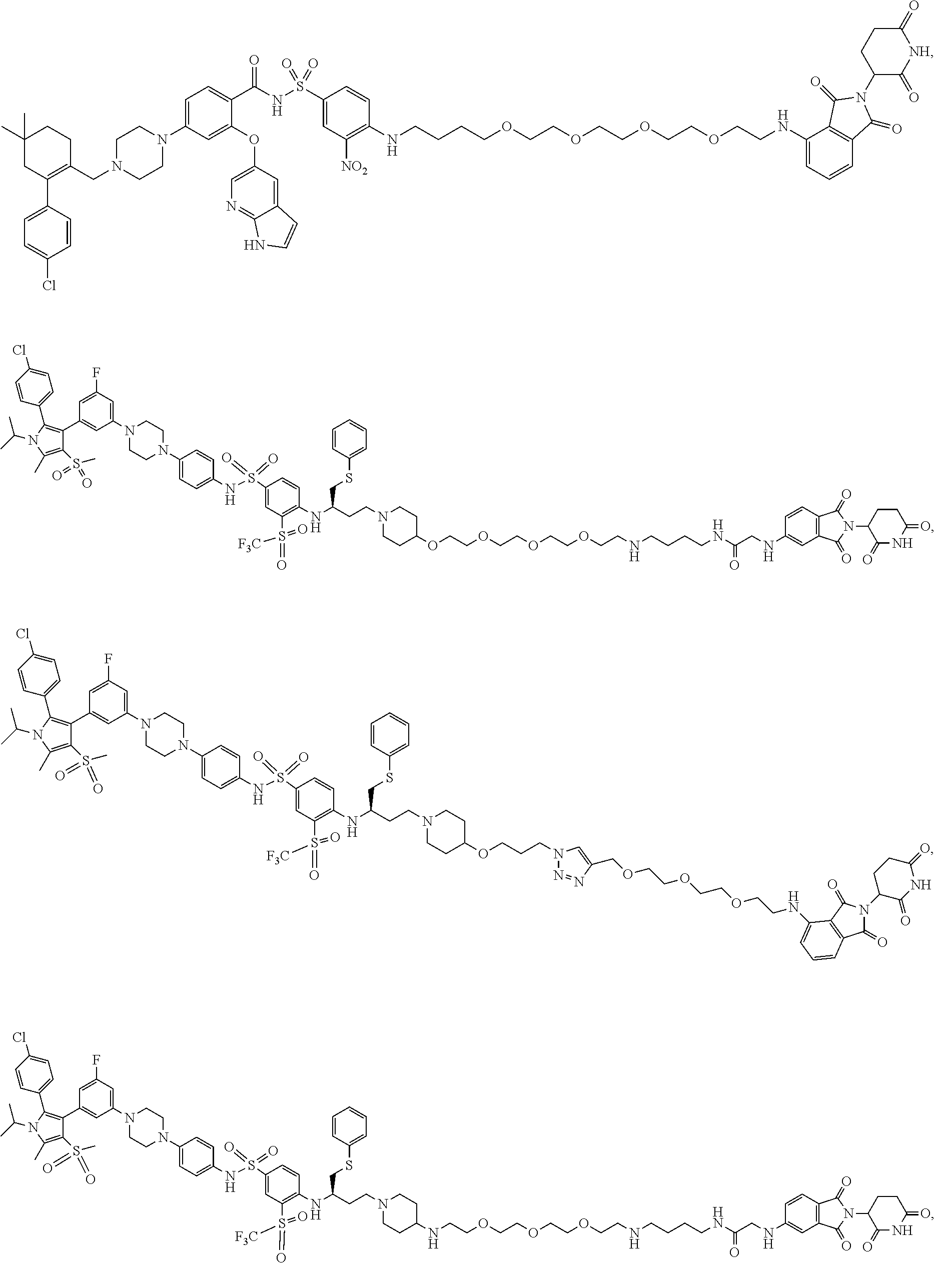

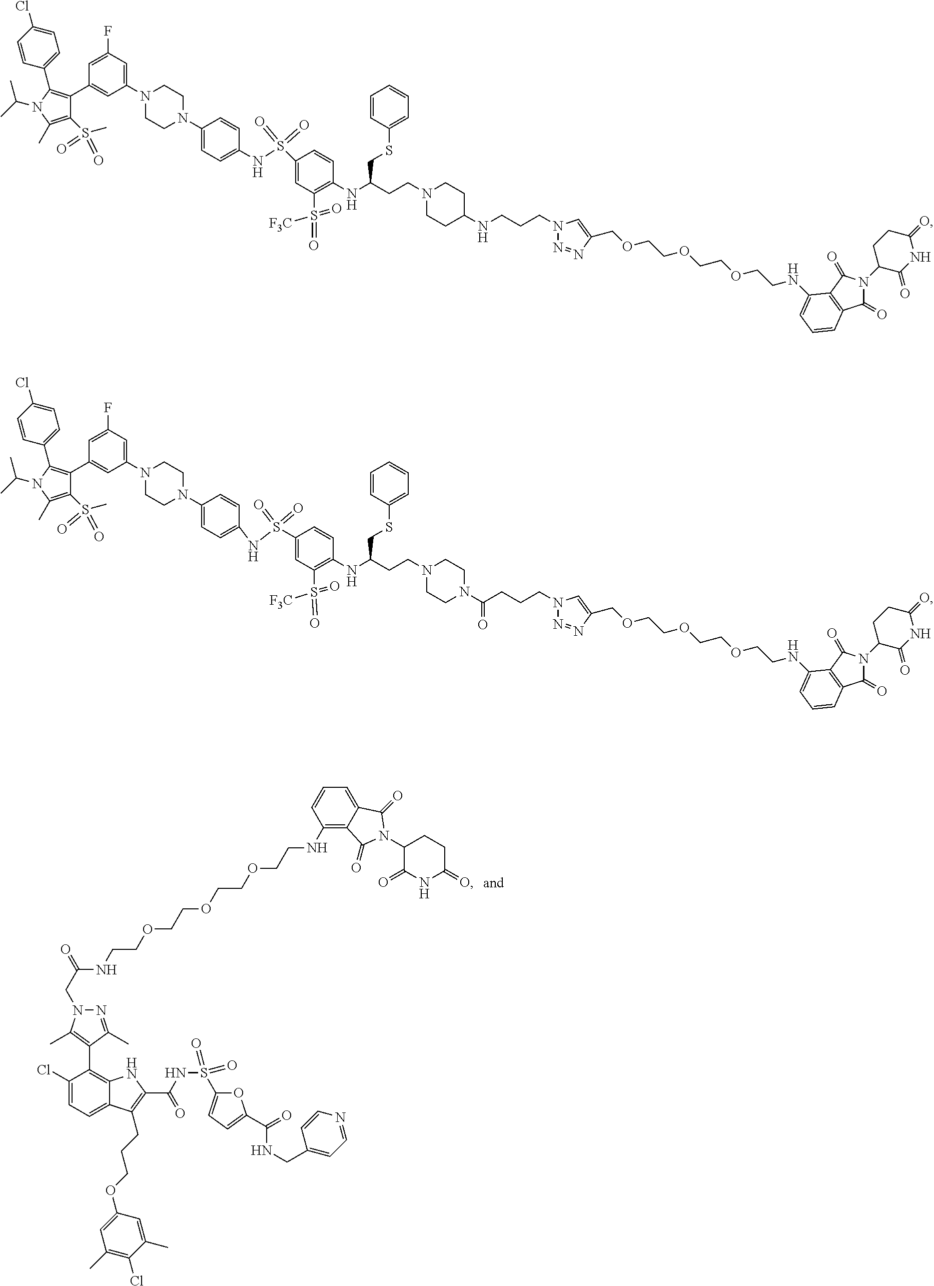

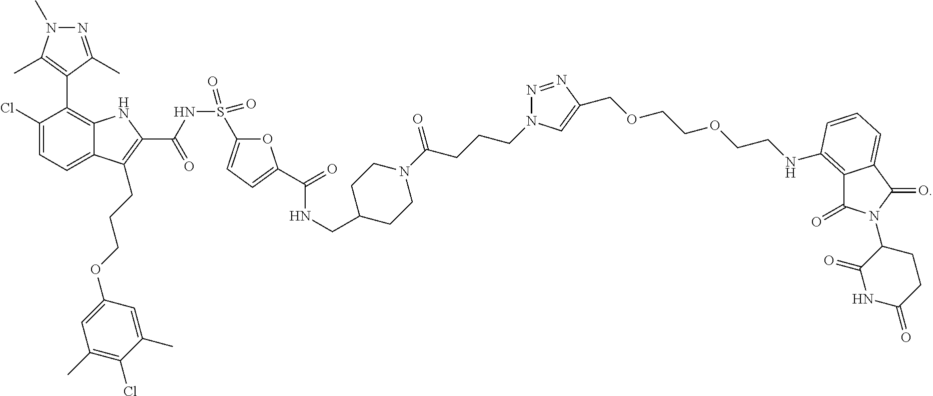 [SMILES: Cc1cc(OCCCc2c(C(=O)NS(=O)(=O)c3ccc(C(=O)NCC4CCN(C(=O)CCCn5cc(COCCOCCNc6cccc7c6C(=O)N(C6CCC(=O)NC6=O)C7=O)nn5)CC4)o3)[nH]c3c(-c4c(C)nn(C)c4C)c(Cl)ccc23)cc(C)c1Cl]

ABT-263 is also known as Navitoclax in the art and is known to be useful as an inhibitor of Bcl-2, Bcl-w and, Bcl-xL. Unfortunately, administration of ABT263 to subjects has been shown to cause thrombocytopenia as platelets also rely on Bcl-xl for survival. Compounds of Formula (I) and Formula (II) of the present invention, through in-part, the selection of a proper E3 ligase ligand, add an extra layer of selectivity to the senolytic agents that result in minimum degradation effects on Bcl-xl proteins in platelets compared with ABT263.

### (c) Senolytic Agents

A senolytic agent as used herein is an agent that “selectively” (preferentially or to a greater degree) destroys, kills, removes, or facilitates selective destruction of senescent cells. In other words, the senolytic agent destroys or kills a senescent cell in a biologically, clinically, and/or statistically significant manner compared with its capability to destroy or kill a non-senescent cell. A senolytic agent is used in an amount and for a time sufficient that selectively kills established senescent cells but is insufficient to kill (destroy, cause the death of) a non-senescent cell in a clinically significant or biologically significant manner. In certain embodiments, the senolytic agents described herein alter at least one signaling pathway in a manner that induces (initiates, stimulates, triggers, activates, promotes) and results in (i.e., causes, leads to) death of the senescent cell. The senolytic agent may alter, for example, either or both of a cell survival signaling pathway (e.g., Akt pathway) or an inflammatory pathway, for example, by antagonizing a protein within the cell survival and/or inflammatory pathway in a senescent cell.

Without wishing to be bound by a particular theory, the mechanism by which the inhibitors and antagonists described herein selectively kill senescent cells is by inducing (activating, stimulating, removing inhibition of) an apoptotic pathway that leads to cell death. Non-senescent cells may be proliferating cells or may be quiescent cells. In certain instances, exposure of non-senescent cells to the senolytic agent as used in the methods described herein may temporarily reduce the capability of non-senescent cell to proliferate; however, an apoptotic pathway is not induced and the non-senescent cell is not destroyed.

Certain senolytic agents that may be used in the methods described herein have been described as useful for treating a cancer; however, in the methods for treating a senescence associated disorder or disease, the senolytic agents are administered in a manner that would be considered different and likely ineffective for treating a cancer. The method used for treating chemotherapy-induced peripheral neuropathy with a senolytic agent described herein may comprise one or more of a decreased daily dose, decreased cumulative dose over a single treatment cycle, or decreased cumulative dose of the agent from multiple treatment cycles than the dose of an agent required for cancer therapy; therefore, the likelihood is decreased that one or more adverse effects (i.e., side effects) will occur, which adverse effects are associated with treating a subject according to a regimen optimized for treating a cancer. In contrast, as a senolytic agent, the compounds described herein may be administered at a lower dose than presently described in the art or in a manner that selectively kill senescent cells (e.g., intermittent dosing). In certain embodiments, the senolytic agents described herein may be administered at a lower cumulative dose per treatment course or treatment cycle that would likely be insufficiently cytotoxic to cancer cells to effectively treat the cancer. In other words, according to the methods described herein, the senolytic agent is not used in a manner that would be understood by a person skilled in the art as a primary therapy for treating a cancer, whether the agent is administered alone or together with one or more additional chemotherapeutic agents or radiotherapy to treat the cancer. Even though an agent as used in the methods described herein is not used in a manner that is sufficient to be considered as a primary cancer therapy, the methods and senolytic combinations described herein may be used in a manner (e.g., a short term course of therapy) that is useful for inhibiting metastases. A “primary therapy for cancer” as used herein means that when an agent, which may be used alone or together with one or more agents, is intended to be or is known to be an efficacious treatment for the cancer as determined by a person skilled in the medical and oncology arts, administration protocols for treatment of the cancer using the agent have been designed to achieve the relevant cancer-related endpoints. To further reduce toxicity, a senolytic agent may be administered at a site proximal to or in contact with senescent cells (not tumor cells). Localized delivery of senolytic agents is described in greater detail herein.

In certain embodiments, a senolytic agent as used in the methods described herein is a small molecule compound. These senolytic agents that are small molecules may also be called herein senolytic compounds. In certain embodiments, the senolytic agents that are small molecules include those that are activated or that are pro-drugs which are converted to the active form by enzymes within the cell. In a more specific embodiment, the enzymes that convert a pro-drug to an active senolytic form are those expressed at a higher level in senescent cells than in non-senescent cells.

Senolytic agents described herein that may alter at least one signaling pathway include an agent that inhibits an activity of at least one of the target proteins within the pathway. The senolytic agent may be a specific inhibitor of one or more BCL-2 anti-apoptotic protein family members wherein the inhibitor inhibits at least BCL-xL (e.g., a Bcl-2/Bcl-xL/Bcl-w inhibitor; a selective Bcl-xL inhibitor; a Bcl-xL/Bcl-w inhibitor); an Akt kinase specific inhibitor; or an MDM2 inhibitor. In other particular embodiments, methods comprise use of at least two senolytic agents wherein at least one senolytic agent and a second senolytic agent are each different and independently alter either one or both of a survival signaling pathway and an inflammatory pathway in a senescent cell.

Senolytic agents that may be used in the methods for treating or preventing a senescence-associated disease or disorder include small organic molecules. Small organic molecules (also called small molecules or small molecule compounds herein) typically have molecular weights less than 105 daltons, less than 104 daltons, or less than 103 daltons. In certain embodiments, a small molecule senolytic agent does not violate the following criteria more than once: (1) no more than 5 hydrogen bond donors (the total number of nitrogen-hydrogen and oxygen-hydrogen bonds); (2) not more than 10 hydrogen bond acceptors (all nitrogen or oxygen atoms); (3) a molecular mass less than 500 daltons; (4) an octanol-water partition coefficient log P not greater than 5.

In certain embodiments, the senolytic agent may be an MDM2 inhibitor. An MDM2 (murine double minute 2) inhibitor that may be used in the methods for selectively killing senescent cells and treating or preventing (i.e., reducing or decreasing the likelihood of occurrence or development of) a senescence-associated disease or disorder may be a small molecule compound that belongs to any one of the following classes of compounds, for example, a cis-imidazoline compound, a spiro-oxindole compound, a benzodiazepine compound, a piperidinone compound, a tryptamine compound, and CGM097, and related analogs. In certain embodiments, the MDM2 inhibitor is also capable of binding to and inhibiting an activity of MDMX (murine double minute X, which is also known as HDMX in humans). The human homolog of MDM2 is called HDM2 (human double minute 2) in the art. Therefore, when a subject treated by the methods described herein is a human subject, the compounds described herein as MDM2 inhibitors also inhibit binding of HDM2 to one or more of its ligands.

Reports have described several activities and biological functions of MDM2. These reported activities include the following: acts as a ubiquitin ligase E3 toward itself and ARRB1; permits nuclear export of p53; promotes proteasome-dependent ubiquitin-independent degradation of retinoblastoma RB1 protein; inhibits DAXX-mediated apoptosis by inducing its ubiquitination and degradation; component of TRIM28/KAP1-MDM2-p53 complex involved in stabilizing p53; component of TRIM28/KAP1-ERBB4-MDM2 complex that links growth factor and DNA damage response pathways; mediates ubiquitination and subsequent proteasome degradation of DYRK2 in the nucleus; ubiquitinates IGF1R and SNAI1 and promotes them to proteasomal degradation. MDM2 has also been reported to induce mono-ubiquitination of the transcription factor FOXO4 (see, e.g., Brenkman et al., PLOS One 3(7):e2819, doi:10.1371/journal.pone.0002819). The MDM2 inhibitors described herein may disrupt the interaction between MDM2 and any one or more of the aforementioned cellular components.

In one embodiment, a compound useful for the methods described herein is a cis-imidazoline small molecule inhibitor. Cis-imidazoline compounds include those called nutlins in the art. Similar to other MDM2 inhibitors described herein, nutlins are cis-imidazoline small molecule inhibitors of the interaction between MDM2 and p53 In certain embodiments, the methods described herein comprise use of a nutlin compound called Nutlin-1; or a nutlin compound called Nutlin-2; or a Nutlin compound called Nutlin-3 (see CAS Registry No. 675576-98-4 and No. 548472-68-0). The active enantiomer of Nutlin-3 (4-[[4S,5R)-4,5-bis(4-chlorophenyl)-4,5-dihydro-2-[4-methoxy-2-(-1-methylethoxy)phenyl]-1H-imidazol-1-yl]carbonyl]-2-piperazinone) is called Nutlin-3a in the art. In certain embodiments, the methods described herein comprise use of Nutlin-3a for selectively killing senescent cells.

Another exemplary cis-imidazoline small molecule compound useful for selectively killing senescent cells is RG-7112 (Roche) (CAS No: 939981-39-2; IUPAC name: ((4S,5R)-2-(4-(tert-butyl)-2-ethoxyphenyl)-4,5-bis(4-chlorophenyl)-4,5-di-methyl-4,5-dihydro-1H-imidazol-1-yl)(4-(3-(methylsulfonyl)propyl)piperazin-1-yl)methanone. In another particular embodiment, the MDM2 inhibitor is a cis-imidazoline compound called RG7338 (Roche) (IPUAC Name: 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoic acid) (CAS 1229705-06-9. Yet another exemplary nutlin compound is RO5503781. Other potent cis-imidazoline small molecule compounds include dihydroimidazothiazole compounds (e.g., DS-3032b; Daiichi Sankyo).

In still other embodiments, a cis-imidazoline compound that may be used in the methods described herein is a dihydroimidazothiazole compound. In other embodiments, the MDM2 small molecule inhibitor is a spiro-oxindole compound. See, for example, MDM2 inhibitors called in the art MI-63, MI-126; MI-122, MI-142, MI-147, MI-18, MI-219, MI-220, MI-221, and MI-773. Another specific spiro-oxindole compound is 3-(4-chlorophenyl)-3-((1-(hydroxymethyl)cyclopropyl)methoxy)-2-(4-nitrobe-nzyl)isoindolin-1-one. Another compound is called MI888.

In still other embodiments, the MDM2 small molecule inhibitor that may be used in the methods described herein is a benzodiazepinedione. Benzodiazepinedione compounds that may be used in the methods described herein include 1,4-benzodiazepin-2,5-dione compounds. Examples of benzodiazepinedione compounds include 5-[(3S)-3-(4-chlorophenyl)-4-[(R)-1-(4-chlorophenyl)ethyl]-2,5-dioxo-7-ph-enyl-1,4-diazepin-1-yl]valeric acid and 5-[(3S)-7-(2-bromophenyl)-3-(4-chlorophenyl)-4-[(R)-1-(4-chlorophenyl)eth-yl]-2,5-dioxo-1,4-diazepin-1-yl]valeric acid. Other benzodiazepinedione compounds are called in the art TDP521252 (IUPAC Name: 5-[(3S)-3-(4-chlorophenyl)-4-[(1R)-1-(4-chlorophenyl)ethyl]-7-ethynyl-2,5-dioxo-3H-1,4-benzodiazepin-1-yl]pentanoic acid) and TDP665759 (IUPAC Name: (3S)-4-[(1R)-1-(2-amino-4-chlorophenyl)ethyl]-3-(4-chlorophenyl)-7-iodo-1-[3-(4-methylpiperazin-1-yl)propyl]-3H-1,4-benzodiazepine-2,5-dione).

In yet another embodiment, the MDM2 small molecule inhibitor is a terphenyl (see, e.g., Yin et al., Angew Chem Int Ed Engl 2005; 44:2704-707; Chen et al., Mol Cancer Ther 2005; 4:1019-25). In yet another specific embodiment, the MDM2 inhibitor that may be used in the methods described herein is a quilinol (see, e.g., Lu et al., J Med Chem 2006; 49:3759-62). In yet another certain embodiment, the MDM2 inhibitor is a chalcone (see, e.g., Stoll et al., Biochemistry 2001; 40:336-44). In yet another particular embodiment, the MDM2 inhibitor is a sulfonamide (e.g., NSC279287) (see, e.g., Galatin et al., J Med Chem 2004; 47:4163-65). In other embodiments, a compound that may be used in the methods described herein is a tryptamine, such as serdemetan (JNJ-26854165; chemical name: N1-(2-(1H-indol-3-yl)ethyl)-N4-(pyridine-4-yl)benzene-1,4-diamine; CAS No. 881202-45-5) (Johnson & Johnson, New Brunswick, N.J.). Serdemetan is a tryptamine derivative that activates p53 and acts as a HDM2. In still other embodiments, the MDM2 inhibitor is a piperidinone compound. An example of a potent MDM2 piperidinone inhibitor is AM-8553 ({(3R,5R,6S)-5-(3-Chlorophenyl)-6-(4-chlorophenyl)-1-[(2S,3S)-2-hydroxy-3-pentanyl]-3-methyl-2-oxo-3-piperidinyl}acetic acid; CAS No. 1352064-70-0) (Amgen, Thousand Oaks, Calif.). In other particular embodiments, an MDM2 inhibitor that may be used in the methods described herein is a piperidine (Merck, Whitehouse Station, N.J.). In other embodiments, an MDM2 inhibitor that may be used in the methods is an imidazole-indole compound (Novartis) (see, e.g., Intl Patent Appl. Publ. No. WO 2008/119741).

Examples of compounds that bind to MDM2 and to MDMX and that may be used in the methods described herein include RO-2443 and RO-5963 ((Z)-2-(4-((6-Chloro-7-methyl-1H-indol-3-yl)methylene)-2,5-dioxoimidazoli-din-1-yl)-2-(3,4-difluorophenyl)-N-(1,3-dihydroxypropan-2-yl)acetamide). In another specific embodiment, an MDM2 inhibitor referred to in the art as CGM097 may be used in the methods described herein for selectively killing senescent cells and for treating a senescence-associated disease or disorder.

In certain embodiments, the senolytic agent may be an inhibitor of one or more proteins in the BCL-2 family. In certain embodiments, the at least one senolytic agent is selected from an inhibitor of one or more BCL-2 anti-apoptotic protein family members wherein the inhibitor inhibits at least BCL-xL. Inhibitors of BCL-2 anti-apoptotic family of proteins alter at least a cell survival pathway. Apoptosis activation may occur via an extrinsic pathway triggered by the activation of cell surface death receptors or an intrinsic pathway triggered by developmental cues and diverse intracellular stresses. This intrinsic pathway, also known as the stress pathway or mitochondrial pathway, is primarily regulated by the BCL-2 family, a class of key regulators of caspase activation consisting of anti-apoptotic (pro-survival) proteins having BH1-BH4 domains (BCL-2 (i.e., the BCL-2 protein member of the BCL-2 anti-apoptotic protein family), BCL-xL, BCL-w, A1, MCL-1, and BCL-B); pro-apoptotic proteins having BH1, BH2, and BH3 domains (BAX, BAK, and BOK); and pro-apoptotic BH3-only proteins (BIK, BAD, BID, BIM, BMF, HRK, NOXA, and PUMA). BCL-2 anti-apoptotic proteins block activation of pro-apoptotic multi-domain proteins BAX and BAK.

As used herein and unless otherwise stated, a BCL-2 family member that is inhibited by the agents described herein is a pro-survival (anti-apoptotic) family member. The senolytic agents used in the methods described herein inhibit one or more functions of the BCL-2 anti-apoptotic protein, BCL-xL (which may also be written herein and in the art as Bcl-xL, BCL-XL, Bcl-xl, or Bcl-XL). In certain embodiments, in addition to inhibiting BCL-xL function, the inhibitor may also interact with and/or inhibit one or more functions of BCL-2 (i.e., BCL-xL/BCL-2 inhibitors). In yet another certain embodiment, senolytic agents used in the methods described herein are classified as inhibitors of each of BCL-xL and BCL-w (i.e., BCL-xL/BCL-w inhibitors). In still another specific embodiment, senolytic agents used in the methods described herein that inhibit BCL-xL may also interact with and inhibit one or more functions of each of BCL-2 (i.e., the BCL-2 protein) and BCL-w (i.e., BCL-xL/BCL-2/BCL-w inhibitors), thereby causing selective killing of senescent cells. In certain embodiments, a BCL-2 anti-apoptotic protein inhibitor interferes with the interaction between the BCL-2 anti-apoptotic protein family member (which includes at least BCL-xL) and one or more ligands or receptors to which the BCL-2 anti-apoptotic protein family member would bind in the absence of the inhibitor. In other particular embodiments, an inhibitor of one or more BCL-2 anti-apoptotic protein family members wherein the inhibitor inhibits at least BCL-xL specifically binds only to one or more of BCL-xL, BCL-2, BCL-w and not to other Bcl-2 anti-apoptotic Bcl-2 family members, such as Mcl-1 and BCL2A1.

In still another embodiment, the senolytic agent used in the methods described herein is a BCL-xL selective inhibitor and inhibits one or more functions of BCL-xL. Such senolytic agents that are BCL-xL selective inhibitors do not inhibit the function of one or more other BCL-2 anti-apoptotic proteins in a biologically or statistically significant manner BCL-xL may also be called BCL2L1, BCL2-like 1, BCLX, BCL2L, BCLxL, or BCL-X herein and in the art. In one embodiment, BCL-xL selective inhibitors alter (e.g., reduce, inhibit, decrease, suppress) one or more functions of BCL-xL but do not significantly inhibit one or more functions of other proteins in the BCL-2 anti-apoptotic protein family (e.g., BCL-2 or BCL-w). In certain embodiments, a BCL-xL selective inhibitor interferes with the interaction between BCL-xL and one or more ligands or receptors to which BCL-xL would bind in the absence of the inhibitor. In certain particular embodiments, a senolytic agent that inhibits one or more of the functions of BCL-xL selectively binds to human BCL-xL but not to other proteins in the BCL-2 family, which effects selective killing of senescent cells.

In certain embodiments, a BCL-xL inhibitor is a selective inhibitor, meaning, that it preferentially binds to BCL-xL over other anti-apoptotic BCL2 family members (e.g., BCL-2, MCL-1, BCL-w, BCL-b, and BFL-1/A1). In certain embodiments, a BCL-XL selective inhibitor exhibits at least a 5-fold, 10-fold, 50-fold, 100-fold, 1000-fold, 10000-fold, 20000-fold, or 30000-fold selectivity for binding a BCL-XL protein or nucleic acid over a BCL-2 protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over a MCL-1 protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over a BCL-w protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-XL protein or nucleic acid over a BCL-B protein or nucleic acid. In certain embodiments, a BCL-XL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over an A1 protein or nucleic acid. As described herein, in certain embodiments, an inhibitor of one or more BCL-2 anti-apoptotic protein family members wherein the inhibitor inhibits at least BCL-xL (e.g., a BCL-xL selective inhibitor) has no detectable binding to MCL-1 or to BCL2A1.

Methods for measuring binding affinity of a BCL-xL inhibitor for BCL-2 family proteins are known in the art. By way of a non-limiting example, binding affinity of a BCL-xL inhibitor may be determined using a competition fluorescence polarization assay in which a fluorescent BAK BH3 domain peptide is incubated with BCL-xL protein (or other BCL-2 family protein) in the presence or absence of increasing concentrations of the BCL-XL inhibitor.

In particular embodiments, the BCL-xL inhibitor is a small molecule compound that belongs to any one of the following classes of compounds, for example, a benzothiazole-hydrazone compound, aminopyridine compound, benzimidazole compound, tetrahydroquinoline compound, and phenoxyl compound and related analogs.

In one embodiment, a BCL-xL selective inhibitor useful for the methods described herein is a benzothiazole-hydrazone small molecule inhibitor. Benzothiazole-hydrazone compounds include WEHI-539 (5-[3-[4-(aminomethyl)phenoxy]propyl]-2-[(8E)-8-(1,3-benzothiazol-2-ylhyd-razinylidene)-6,7-dihydro-5H-naphthalen-2-yl]-1,3-thiazole-4-carboxylic acid), a BH3 peptide mimetic that selectively targets BCL-xL (see, e.g., Lessene et al., Nature Chemical Biology 9:390-397 (2013)). In certain embodiments, the methods described herein comprise use of WEHI-539 for selectively killing senescent cells.

In other embodiments, the BCL-xL selective inhibitor is an aminopyridine compound. An aminopyridine compound that may be used as a selective BCL-xL inhibitor is BXI-61 (3-[(9-amino-7-ethoxyacridin-3-yl)diazenyl]pyridine-2,6-diamine). In certain embodiments, the methods described herein comprise use of BXI-61 for selectively killing senescent cells. In still other embodiments, the BCL-xL selective inhibitor that may be used in the methods described herein is a benzimidazole compound. An example of a benzimidazole compound that may be used as a selective BCL-XL inhibitor is BXI-72 (2′-(4-Hydroxyphenyl)-5-(4-methyl-1-piperazinyl)-2,5′-bi(1H-benzimidazole-) trihydrochloride). In certain embodiments, the methods described herein comprise use of BXI-72 for selectively killing senescent cells. In yet another embodiment, the BCL-xL selective inhibitor is a tetrahydroquinoline compound (see, e.g., U.S. Patent Publ. No. 2014-0005190).

In other embodiments, a BCL-xL selective inhibitor is a phenoxyl compound. An example of a phenoxyl compound that may be used as a selective BCL-xL inhibitor is 2[[3-(2,3-dichlorophenoxy) propyl]amino]ethanol (2,3-DCPE). In certain embodiments, the methods described herein comprise use of 2,3-DCPE for selectively killing senescent cells.

In still another embodiment, an inhibitor of a Bcl-2 anti-apoptotic family member that inhibits at least BCL-xL is described in U.S. Pat. No. 8,232,273. In a particular embodiment, the inhibitor is a BCL-xL selective inhibitor called A-1155463 (see, e.g., Tao et al., ACS Med. Chem. Lett., 2014, 5(10). In still another embodiment, the senolytic agent is a compound that induces degradation of a Bcl-2 anti-apoptotic family member such as those described above in section (I)(a) and section (I)(b).

In other embodiments, a senolytic agent of interest inhibits other BCL-2 anti-apoptotic family members in addition to BCL-xL. For example, methods described herein comprise use of BCL-xL/BCL-2 inhibitors, BCL-xL/BCL-2/BCL-w inhibitors, and BCL-xL/BCL-w inhibitors and analogs thereof. In certain embodiments, the inhibitors include compounds that inhibit BCL-2 and BCL-xL, which inhibitors may also inhibit BCL-w. Examples of these inhibitors include ABT-263 (4-[4-[[2-(4-chlorophenyl)-5,5-dimethylcyclohexen-1-yl]methyl]piperazin-1-yl]-N44-[[(2R)-4-morpholin-4-yl-1-phenylsulfanylbutan-2-yl]amino]-3-(tri-fluoromethylsulfonyl)phenyl]sulfonylbenzamide or IUPAC, (R)-4-(4-((4′-chloro-4,4-dimethyl-3,4,5,6-tetrahydro-[1,1′-biphenyl]-2-yl-)methyl)piperazin-1-yl)-N-((4-((4-morpholino-1-(phenylthio)butan-2-yl)amin-o)-3-((trifluoromethyl)sulfonyl)phenyl)sulfonyl)benzamide) and ABT-737 (4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfo-nyl]benzamide, Benzamide, 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfon-yl]- or 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfony-(benzamide). In other embodiments, the BCL-2 anti-apoptotic protein inhibitor is a quinazoline sulfonamide compound. In still another embodiment, the BCL-2 anti-apoptotic protein inhibitor is a small molecule compound as described in Zhou et al., J. Med. Chem., 2012, 55:4664 (see, e.g., Compound 21 (R)-4-(4-chlorophenyl)-3-(3-(4-(4-(4-((4-(dimethylamino)-1-(phenylthio)bu-tan-2-yl)amino)-3-nitrophenylsulfonamido)phenyl)piperazin-1-yl)phenyl)-5-e-thyl-1-methyl-1H-pyrrole-2-carboxylic acid) and Zhou et al., J. Med. Chem., 2012, 55:6149 (see, e.g., Compound 14 (R)-5-(4-Chlorophenyl)-4-(3-(4-(4-(4-((4-(dimethylamino)-1-(phenylthio)bu-tan-2-yl)amino)-3-nitrophenylsulfonamido)phenyl)piperazin-1-yl)phenyl)-1-e-thyl-2-methyl-1H-pyrrole-3-carboxylic acid; Compound 15 (R)-5-(4-Chlorophenyl)-4-(3-(4-(4-(4-(4-(dimethylamino)-1-(phenylthio)but-an-2-yl)amino)-3-nitrophenylsulfonamido)phenyl)piperazin-1-yl)phenyl)-1-is-opropyl-2-methyl-1H-pyrrole-3-carboxylic acid). In other embodiments, the BCL-2 anti-apoptotic protein inhibitor is a BCL-2/BCL-xL inhibitor such as BM-1074; BM-1197; N-acylsufonamide compounds. In still another embodiment, the BCL-2 anti-apoptotic protein inhibitor is a small molecule macrocyclic compound. In yet another embodiment, the BCL-2 anti-apoptotic protein inhibitor is an isoxazolidine compound.

In certain embodiments, the senolytic agent is a compound that is an inhibitor of Bcl-2, Bcl-w, and Bcl-xL, such as ABT-263 or ABT-737. In certain specific embodiments, the senolytic agent is a compound or a pharmaceutically acceptable salt, stereoisomer, tautomer, or prodrug thereof as illustrated below, which depicts the structure of ABT-263. ABT-263 is also known as Navitoclax in the art.

In certain embodiments the senolytic agent is an Akt Kinase inhibitor. For example, a senolytic agent can be a small molecule compound and analogs thereof that inhibits Akt. In some embodiments, the senolytic agent is a compound that selectively inhibits Akt1, Akt2, and Akt3, relative to other protein kinases.

Akt inhibitors (which may also be called Akt kinase inhibitors or AKT kinase inhibitors) can be divided into six major classes based on their mechanisms of action. Akt is also called protein kinase B (PKB) in the art. The first class contains ATP competitive inhibitors of Akt and includes compounds such as CCT128930 and GDC-0068, which inhibit Akt2 and Akt1. This category also includes the pan-Akt kinase inhibitors such as GSK2110183 (afuresertib), GSK690693, and AT7867. The second class contains lipid-based Akt inhibitors that act by inhibiting the generation of PIP3 by PI3K. This mechanism is employed by phosphatidylinositol analogs such as Calbiochem Akt Inhibitors I, II and III or other PI3K inhibitors such as PX-866. This category also includes compounds such as Perifosine (KRX-0401) (Aeterna Zentaris/Keryx). The third class contains a group of compounds called pseudosubstrate inhibitors. These include compounds such as AKTide-2 T and FOXO3 hybrid. The fourth class consists of allosteric inhibitors of AKT kinase domain, and include compounds such as MK-2206 (8-[4-(1-aminocyclobutyl)phenyl]-9-phenyl-2H-[1,2,4]triazolo[3,4-f][1,6]n-aphthyridin-3-one; dihydrochloride) (Merck & Co.). The fifth class consists of antibodies and include molecules such as GST-anti-Akt1-MTS. The last class comprises compounds that interact with the PH domain of Akt, and includes Triciribine and PX-316. Other compounds described in the art that act as AKT inhibitors include, for example, GSK-2141795 (GlaxoSmithKline), VQD-002, miltefosine, AZD5363, GDC-0068, and API-1. Techniques for determining the activity of AKT inhibitors are routinely practiced by persons skilled in the art

In certain embodiments, at least one senolytic agent may be administered with at least one other senolytic agent, which two or more senolytic agents act additively or synergistically to selectively kill senescent cells. In particular embodiments, methods are provided for using a senolytic agent wherein the senolytic agent alters either a cell survival signaling pathway or an inflammatory pathway or alters both the cell survival signaling pathway and the inflammatory pathway in a senescent cell. In other particular embodiments, methods comprise use of at least two senolytic agents wherein at least one senolytic agent and a second senolytic agent are each different and independently alter either one or both of a survival signaling pathway and an inflammatory pathway in a senescent cell. The adjectives, first, second, third, and such, in this context are used for convenience only and are not to be construed as describing order or administration, preference, or level of senolytic activity or other parameter unless expressly described otherwise. In particular embodiments, when two or more senolytic agents are used in the methods described herein, each senolytic agent is a small molecule.

The small molecule compounds described herein as senolytic agents include physiologically acceptable salts (i.e., pharmaceutically acceptable salts), hydrates, solvates, polymorphs, metabolites, and prodrugs of the senolytic agents. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

The compounds described herein may generally be used as the free acid or free base. Alternatively, the compounds may be used in the form of acid or base addition salts. Acid addition salts of the free base amino compounds may be prepared according to methods well known in the art, and may be formed from organic and inorganic acids. Suitable organic acids include (but are not limited to) maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, malonic, and benzenesulfonic acids. Suitable inorganic acids include (but are not limited to) hydrochloric, hydrobromic, sulfuric, phosphoric, and nitric acids. Base addition salts of the free acid compounds of the compounds described herein may also be prepared by methods well known in the art, and may be formed from organic and inorganic bases. Additional salts include those in which the counterion is a cation. Suitable inorganic bases included (but are not limited to) the hydroxide or other salt of sodium, potassium, lithium, ammonium, calcium, barium, magnesium, iron, zinc, copper, manganese, aluminum, and the like, and organic bases such as substituted ammonium salts (for example, dibenzylammonium, benzylammonium, 2-hydroxyethylammonium). Further salts include those in which the counterion is an anion, such as adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate. Thus, the term “pharmaceutically acceptable salt” of compounds described herein is intended to encompass any and all pharmaceutically suitable salt forms.

Compounds may sometimes be depicted as an anionic species. One of ordinary skill in the art will recognize that the compounds exist with an equimolar ratio of cation. For instance, the compounds described herein can exist in the fully protonated form, or in the form of a salt such as sodium, potassium, ammonium or in combination with any inorganic base as described above. When more than one anionic species is depicted, each anionic species may independently exist as either the protonated species or as the salt species. In some specific embodiments, the compounds described herein exist as the sodium salt. In other specific embodiments, the compounds described herein exist as the potassium salt.

Furthermore, some of the crystalline forms of any compound described herein may exist as polymorphs, which are also included and contemplated by the present disclosure. In addition, some of the compounds may form solvates with water or other organic solvents. Often crystallizations produce a solvate of the disclosed compounds. As used herein, the term “solvate” refers to an aggregate that comprises one or more molecules of any of the disclosed compounds with one or more molecules of solvent. The solvent may be water, in which case the solvate may be a hydrate. Alternatively, the solvent may be an organic solvent. Thus, the presently disclosed compounds may exist as a hydrate, including a monohydrate, dihydrate, hemihydrate, sesquihydrate, trihydrate, tetrahydrate and the like, as well as the corresponding solvated forms. Certain embodiments of the compounds may be true solvates, while in other instances, some embodiments of the compounds may merely retain adventitious water or be a mixture of water plus some adventitious solvent.

In general, the compounds used in the methods described herein may be made according to organic synthesis techniques known to those skilled in this art, starting from commercially available chemicals and/or from compounds described in the chemical literature. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002. Methods known to one of ordinary skill in the art may be identified through various reference books and databases. Suitable reference books and treatises detail the synthesis of reactants useful in the preparation of compounds described herein, or provide references to articles that describe the preparation.

### (d) Components of the Composition

The present disclosure also provides pharmaceutical compositions. The pharmaceutical compositions comprise a compound of Formula (I), a compound of Formula (II), a senolytic agent or a combination thereof, as an active ingredient and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising a compound of Formula (I), a compound of Formula (II), a senolytic agent or a combination thereof, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery a compound of Formula (I), a compound of Formula (II), a senolytic agent or a combination thereof, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, a compound of Formula (I), a compound of Formula (II), a senolytic agent or a combination thereof may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a compound of Formula (I) or a compound of Formula (II) (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The compound of Formula (I), compound of Formula (II), senolytic agent or a combination thereof, may be encapsulated in a microemulsion by any method generally known in the art.

### (d) Additional Compounds

In an aspect, the composition further comprises at least one or more anticancer therapeutics.

A chemotherapeutic agent refers to a chemical compound that is useful in the treatment of cancer. The compound may be a cytotoxic agent that affects rapidly dividing cells in general, or it may be a targeted therapeutic agent that affects the deregulated proteins of cancer cells. The chemotherapeutic agent may be an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Suitable anti-metabolites include, but are not limited to aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcetabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Non-limiting examples of suitable anti-tumor antibiotics include aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin.

Non-limiting examples of suitable anti-cytoskeletal agents include cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine.

Suitable topoisomerase inhibitors include, but are not limited to, amsacrine, etoposide (VP-16), irinotecan, mitoxantrone, RFS 2000, teniposide, and topotecan.

Non-limiting examples of suitable anti-hormonal agents such as aminoglutethimide, antiestrogen, aromatase inhibiting 4(5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, and trilostane.

Examples of targeted therapeutic agents include, without limit, monoclonal antibodies such as alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, and vandetanib;

angiogeneisis inhibitors such as angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, and thalidomide; and growth inhibitory polypeptides such as bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1 BB ligand, and Apo-1 ligand.

Non-limiting examples of photodynamic therapeutic agents include aminolevulinic acid, methyl aminolevulinate, retinoids (alitretinon, tamibarotene, tretinoin), and temoporfin.

Other antineoplastic agents include anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, and vorinostat.

Also included are pharmaceutically acceptable salts, acids, or derivatives of any of the above listed agents. The mode of administration of the chemotherapeutic agent can and will vary depending upon the agent and the type of tumor or neoplasm. Suitable modes of administration were detailed in Section II(d), below. A skilled practitioner will be able to determine the appropriate dose of the chemotherapeutic agent.

### II. Methods

The present disclosure encompasses a method of selectively killing one or more senescent cells in a sample, the method comprising contacting a composition comprising an effective amount of a compound of Formula (I), a compound of Formula (II), a senolytic agent or a combination thereof, with the sample. In another aspect, the present disclosure encompasses a method of selectively killing one or more senescent cells in a subject in need thereof, the method comprising administering to the subject a composition comprising a therapeutically effective amount of a compound of Formula (I), a compound of Formula (II), a senolytic agent or a combination thereof. In preferred embodiments, the composition comprising an effective amount of a compound of Formula (I) or a compound of Formula (II) are less toxic to platelets when compared to a control sample or subject treated with ABT-263.

The present disclosure encompasses a method of selectively killing one or more cancer cells in a sample, the method comprising contacting a composition comprising an effective amount of a compound of Formula (I), a compound of Formula (II), a senolytic agent or a combination thereof with the sample. In another aspect, the present disclosure encompasses a method of selectively killing one or more cancer cells in a subject in need thereof, the method comprising administering to the subject a composition comprising a therapeutically effective amount of a compound of Formula (I) or a compound of Formula (II).

The present disclosure encompasses a method of decreasing the senescence associated secretory phenotype of osteoblasts and/or osteocytes in a sample, the method comprising contacting a composition comprising an effective amount of a compound of Formula (I) or a compound of Formula (II) with the sample. In another aspect, the present disclosure encompasses a method of decreasing the senescence associated secretory phenotype of osteoblasts and/or osteocytes in a subject in need thereof, the method comprising administering to the subject a composition comprising a therapeutically effective amount of a compound of Formula (I) or a compound of Formula (II). In preferred embodiments, the composition comprising an effective amount of a compound of Formula (I) or a compound of Formula (II) are less toxic to platelets when compared to a control sample or subject treated with ABT-263.

The present disclosure encompasses a method of decreasing osteoclastogenesis in a sample, the method comprising contacting a composition comprising an effective amount of a compound of Formula (I) or a compound of Formula (II) with the sample. In another aspect, the present disclosure encompasses a method of decreasing osteoclastogenesis in a subject in need thereof, the method comprising administering to the subject a composition comprising a therapeutically effective amount of a compound of Formula (I) or a compound of Formula (II). In preferred embodiments, the composition comprising an effective amount of a compound of Formula (I) or a compound of Formula (II) are less toxic to platelets when compared to a control sample or subject treated with ABT-263.

By selectively killing one or more senescent cells is meant a composition of the invention does not appreciably kill non-senescent cells at the same concentration. Accordingly, the median lethal dose or LD50 of the inhibitor in non-senescent cells may be about 5 to about 50 times higher than the LD50 of the inhibitor in senescent cells. As used herein, the LD50 is the concentration of inhibitor required to kill half the cells in the cell sample. For example, the LD50 of the inhibitor in non-senescent cells may be greater than about 5, about 6, about 7, about 8, about 9 or about 10 times higher than the LD50 of the inhibitor in senescent cells. Alternatively, the LD50 of the inhibitor in non-senescent cells may be greater than about 10, about 15, about 20, about 25, about 30, about 35, about 40, about 45, or about 50 times higher than the LD50 of the inhibitor in senescent cells. Additionally, the LD50 of the inhibitor in non-senescent cells may be greater than 50 times higher than the LD50 of the inhibitor in senescent cells. In a specific embodiment, the LD50 of the inhibitor in non-senescent cells is greater than 10 times higher than the LD500 of the inhibitor in senescent cells. In another specific embodiment, the LD50 of the inhibitor in non-senescent cells is greater than 20 times higher than the LD50 of the inhibitor in senescent cells.

The progression from an actively dividing cell to a metabolically active, non-dividing cell is termed “senescence” or “cellular senescence.” As used herein, the terms “senescence” and “cellular senescence” may be used interchangeably. The term “senescence” also refers to the state into which cells enter after multiple rounds of division and, as a result of cellular pathways, future cell division is prevented from occurring even though the cell remains metabolically active. Senescent cells may differ from their pre-senescent counterparts in one or more of the following ways: 1) they arrest growth and cannot be stimulated to reenter the cell cycle by physiological mitogens; 2) they become resistant to apoptotic cell death; and/or 3) they acquire altered differentiated functions.

In contrast to cancer cells which grow and divide uncontrollably, the ability of most differentiated eukaryotic cells to proliferate is finite. Stated another way, normal cells have an intrinsically determined limit to the number of cell divisions through which they can proceed. This phenomenon has been termed “replicative cellular senescence” and is an intrinsic anticancer mechanism that limits a cell's proliferative ability, thereby preventing neoplastic transformation. Another form of senescence is “premature cellular senescence.” Premature cellular senescence, like replicative cellular senescence, is a terminal fate of mitotic cells, characterized by permanent cell cycle arrest. Unlike replicative cellular senescence, however, premature cellular senescence does not require telomere deterioration and can be induced by a variety of stressors including, but not limited to, ultraviolet light, reactive oxygen species, chemotherapeutics, environmental toxin, cigarette smoking, ionizing radiation, distortion of chromatin structure, excessive mitogenic signaling, and oncogenic mutations. Still another form of senescence is therapy-induced senescence (TIS) which refers to the phenomenon of a subset of tumor cells being forced into a senescent state by therapeutic agents. TIS is known to develop because of certain treatments, including radiotherapy and chemotherapy.

The number of senescent cells in various organs and tissues of a subject increases with age. The accumulation of senescent cells may drive the deterioration that underlies aging and age-related diseases. For example, the accumulation of senescent cells in aged tissue may contribute to age-associated tissue dysfunction, reduced regenerative capacity, and disease. In this context, senescence is considered deleterious because it contributes to decrements in tissue renewal and function. As a non-limiting example, an aged tissue may lack the ability to respond to stress when proliferation is required thereby resulting in the reduced fitness seen with aging. A key component of this model is that substantial numbers of senescent cells should be present in tissues with aging, without, or prior to, pathology.

### (a) Senescent Cells

A senescent cell may be a cell that ceases to divide but remains metabolically active. The non-dividing cells may remain viable for many weeks, but fail to grow/replicate DNA despite the presence of ample space, nutrients and growth factors in the medium. Thus, the senescence growth arrest is essentially permanent because senescent cells cannot be stimulated to proliferate by known physiological stimuli. Further, a senescent cell of the invention may be resistant to certain apoptotic signals and may acquire widespread changes in gene expression. The resistance to apoptosis may explain the increase in senescent cells with age. Manipulation of pro- and anti-apoptotic proteins may cause cells that are destined to die by apoptosis to senesce and, conversely, cause cells that are destined to senesce to undergo apoptosis.

A senescent cell of the invention may be senescent due to replicative cellular senescence, premature cellular senescence or therapy-induced senescence. Senescent cells that are senescent due to replication may have undergone greater than 60 population doublings. Alternatively, senescent cells that are senescent due to replication may have undergone greater than 40, greater than 50, greater than 60, greater than 70 or greater than 80 population doublings. A senescent cell that is prematurely cellular senescent may be induced by, but not limited to, ultraviolet light, reactive oxygen species, chemotherapeutics, environmental toxin, cigarette smoking, ionizing radiation, distortion of chromatin structure, excessive mitogenic signaling, and oncogenic mutations. In a specific embodiment, premature cellular senescence may be induced by ionizing radiation (IR). In another specific embodiment, premature cellular senescence may also be induced by ectopic transfection with Ras oncogene. A senescent cell that is therapy-induced senescent may have been exposed to DNA-damaging therapy.

A senescent cell of the invention may generally be a eurkaryotic cell. Non-limiting examples of senescent cells may include, but are not limited to, mammary epithelial cells, keratinocytes, cardiac myocytes, chondrocytes, endothelial cells (large vessels), endothelial cells (microvascular), epithelial cells, fibroblasts, follicle dermal papilla cells, hepatocytes, melanocytes, osteoblasts, preadipocytes, primary cells of the immune system, skeletal muscle cells, smooth muscle cells, adipocytes, neurons, glial cells, contractile cells, exocrine secretory epithelial cells, extracellular matrix cells, hormone secreting cells, keratinizing epithelial cells, islet cells, lens cells, mesenchymal stem cells, pancreatic acinar cells, paneth cells of the small intestine, primary cells of hemopoietic linage, primary cells of the nervous system, sense organ and peripheral neuron supporting cells, wet stratified barrier epithelial cells and stem cells. In a specific embodiment, the stem cells are adult stem cells. Adult stem cells are stem cells which maintain and repair the tissue in which they are found and are generally referred to by their tissue of origin. Non-limiting examples of adult stem cells include muscle stem cells, hematopoietic stem cells, heart stem cells, neural stem cells, mesenchymal stem cells, intestinal stem cells, skin stem cells, adipose-derived stem cells, endothelial stem cells, and dental pulp stem cells. In a specific embodiment, a senescent cell of the invention is a fibroblast. In another specific embodiment, a senescent cell may be a hematopoietic stem cell.

Further, a senescent cell of the invention may be found in renewable tissues, including the vasculature, hematopoietic system, epithelial organs and the stroma. A senescent cell of the invention may also be found at sites of aging or chronic age-related pathology, such as osteoarthritis and atherosclerosis. Further, a senescent cell of the invention may be associated with benign dysplastic or preneoplastic lesions and benign prostatic hyperplasia. In an embodiment, a senescent cell of the invention may be found in normal and tumor tissues following DNA-damaging therapy. In a specific embodiment, a senescent cell may be found at a site of aging or age-related pathology.

An age-related pathology may include any disease or condition which is fully or partially mediated by the induction or maintenance of a non-proliferating or senescent state in a cell or a population of cells in a subject. Non-limiting examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as progeroid syndromes (i.e., Hutchinson-Gilford progeria syndrome, Werner syndrome, Bloom syndrome, Rothmund-Thomson Syndrome, Cockayne syndrome, xeroderma pigmentosum, trichothiodystrophy, combined xeroderma pigmentosum-Cockayne syndrome, and restrictive dermopathy), ataxia telangiectasia, Fanconi anemia, Friedreich's ataxia, dyskeratosis congenital, aplastic anemia, IPF, and others. A method of identifying an age-related disease or condition as described herein may include detecting the presence of senescent cells.

### (b) Detecting Senescent Cells

In an aspect, a method of the invention may comprise detecting senescent cells. Senescent cells may be detected in vivo or in vitro. Suitable markers for detecting senescent cells in vitro and in vivo are known in the art. For example, methods to detect senescent cells may include, but are not limited to, detecting lack of DNA replication by incorporation of a DNA-staining reagent (e.g., 5-bromodeoxyuridine (BrdU), 3H-thymidine), immunostaining for proteins such as proliferating cell nuclear antigen (PCNA) and Ki-67, histochemical staining for senescence-associated β-galactosidase (SA-β-gal), detecting expression of p16, p19, Pail, Igfbp2, IL-6, Mmp13, Nrgl, differentiated embryo-chondrocyte expressed-1 (DEC1), p15 (a CDK1) and decoy death receptor-2 (DCR2), detecting cytological markers such as senescence-associated heterochromatin foci (SAHFs) and senescence-associated DNA-damage foci (SDFs). SAHFs may be detected by the preferential binding of DNA dyes, such as 4′,6-diamidino-2-phenylindole (DAPI), and the presence of certain heterochromatin-associated histone modifications (e.g., H3 Lys9 methylation) and proteins (e.g., heterochromatin protein-1 (HP1)). Additionally, senescent cells may be detected as described in U.S. Pat. No. 5,491,069 and US Patent Application No. 2010/0086941. In certain embodiments, senescent cells are detected by histochemical staining for SA-β-gal.

In certain embodiments, one or more senescent cells are detected in a sample. A sample may be a cell sample, a tissue sample, or a biopsy from a subject. Generally speaking, a sample may be dependent on the age-related pathology. For instance, a sample may be tissue biopsy material. As such, a tissue sample may be from esophagus, stomach, liver, gallbladder, pancreas, adrenal glands, bladder, gallbladder, large intestine, small intestine, kidneys, liver, pancreas, colon, stomach, thymus, spleen, brain, spinal cord, nerves, adipose tissue, heart, lungs, eyes, corneal, skin or islet tissue or organs. In a specific embodiment, a tissue sample may be from lung, skeletal muscle, and brain. In another specific embodiment, a tissue sample may be from liver and heart. Alternatively, a sample may be a cell sample. As such, a cell sample may be oocytes and/or spermatozoa, mesenchymal stem cells, adipocytes, central nervous system neurons and glial cells, contractile cells, exocrine secretory epithelial cells, extracellular matrix cells, hormone secreting cells, keratinizing epithelial cells, islet cells, kidney cells, lens cells, pancreatic acinar cells, paneth cells of small intestine, primary cells of hemopoietic lineage, primary cells of the nervous system, sense organ and peripheral neuron supporting cells or wet stratified barrier epithelial cells. Detection of senescent cells may be used to assess the replicative history of tissues, thereby providing a method for evaluation of the physiological, in contrast to the chronological age of the tissue.

The number of senescent cells may increase with age. The number of senescent cells in a tissue or sample may be from less than 1% to greater than 15%. In an embodiment, the number of senescent cells in a tissue or sample may be less than 1%, less than 2%, less than 3%, less than 4%, or less than 5%. In another embodiment, the number of senescent cells in a tissue or sample may be about 5%, about 6%, about 7%, about 8%, about 9%, or about 10%. In still another embodiment, the number of senescent cells in a tissue or sample may be greater than 10%, greater than 11%, greater than 12%, greater than 13%, greater than 14%, or greater than 15%.

### (c) Measuring Cell Death

In an aspect, a method of the invention may comprise measuring cell death of senescent cells. Methods of measuring cell death are known in the art. For example, cell death may be measured by Giemsa staining, trypan blue exclusion, acridine orange/ethidium bromide (AO/EB) double staining for fluorescence microscopy and flow cytometry, propidium iodide (PI) staining, annexin V assay, TUNEL assay, DNA ladder, LDH activity, and MTT assay. In a preferred embodiment, cell death is due to induction of apoptosis. Cell death due to induction of apoptosis may be measured by observation of morphological characteristics including cell shrinkage, cytoplasmic condensation, chromatin segregation and condensation, membrane blebbing, and the formation of membrane-bound apoptotic bodies. Cell death due to induction of apoptosis may be measured by observation of biochemical hallmarks including internucleosomal DNA cleavage into oligonucleosome-length fragments. Traditional cell-based methods of measuring cell death due to induction of apoptosis include light and electron microscopy, vital dyes, and nuclear stains. Biochemical methods include DNA laddering, lactate dehydrogenase enzyme release, and MTT/XTT enzyme activity. Additionally, terminal deoxynucleotidyl transferase-mediated dUTP-biotin nick end labeling of DNA fragments (TUNEL) and in situ end labeling (ISEL) techniques are used, which when used in conjunction with standard flow cytometric staining methods yield informative data relating cell death to various cellular parameters, including cell cycle and cell phenotype. See Loo and Rillema, Methods Cell Biol. 1998; 57:251-64, which is incorporated herein by reference, for a review of these methods. In an exemplary embodiment, cell death due to apoptosis may be measured by the reduction of procaspase-3. Caspase-3 has been implicated as an “effector” caspase associated with the initiation of the “death cascade” and is therefore an important marker of the cell's entry point into the apoptotic signaling pathway. Caspase-3 is activated by the upstream caspase-8 and caspase-9, and since it serves as a convergence point for different signaling pathways, it is well suited as a read-out in an apoptosis assay.

The results of these methods may be used to determine the percentage of viable cells. In a preferred embodiment, cell death may be measured as a reduction in viable cells. Since a composition of the invention selectively kills senescent cells, a reduction in viable cells is indicative of a reduction in senescent cells. As described in Section III(b), the number of senescent cells in a sample may be from less than 1% to greater than 15%. As such, a reduction in viable cells following administration of an inhibitor of the invention may be greater than 15% to less than 1%. For example, the reduction in viable cells may be less than 1%, less than 2%, less than 3%, less than 4%, or less than 5%. Alternatively, the reduction in viable cells may be about 5%, about 6%, about 7%, about 8%, about 9%, or about 10%. Additionally, the reduction in viable cells may be greater than 10%, greater than 11%, greater than 12%, greater than 13%, greater than 14%, or greater than 15%.

### (d) Administration

In certain aspects, a therapeutically effective amount of a composition of the invention may be administered to a subject. Administration is performed using standard effective techniques, including peripherally (i.e., not by administration into the central nervous system) or locally to the central nervous system. Peripheral administration includes but is not limited to oral, inhalation, intravenous, intraperitoneal, intra-articular, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. The route of administration may be dictated by the disease or condition to be treated. For example, if the disease or condition is COPD or IPF, the composition may be administered via inhalation. Alternatively, if the disease or condition is osteoarthritis, then the composition may be administered via intra-articular invention. It is within the skill of one in the art, to determine the route of administration based on the disease or condition to be treated. In a specific embodiment, a composition of the invention is administered orally.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

For therapeutic applications, a therapeutically effective amount of a composition of the invention is administered to a subject. A “therapeutically effective amount” is an amount of the therapeutic composition sufficient to produce a measurable response (e.g., cell death of senescent cells, an anti-aging response, an improvement in symptoms associated with a degenerative disease, or an improvement in symptoms associated with a function-decreasing disorder). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, age, the age-related disease or condition, the degenerative disease, the function-decreasing disorder, the symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For example, in one aspect, a composition comprising an effective amount of a compound of Formula (I), a compound of Formula (II), a senolytic agent or a combination thereof is administered directly to the target tissue or organ comprising senescent cells that contribute to manifestation of the disease or disorder. In specific embodiments when treating osteoarthritis, a composition comprising an effective amount of a compound of Formula (I), a compound of Formula (II), a senolytic agent or a combination thereof is administered directly to an osteoarthritic joint (i.e., intra-articularly) of a subject in need thereof. In other specific embodiments, a composition comprising an effective amount of a compound of Formula (I), a compound of Formula (II), a senolytic agent or a combination thereof may be administered to the joint via topical, transdermal, intradermal, or subcutaneous route. In other certain embodiments, methods are provided herein for treating a cardiovascular disease or disorder associated with arteriosclerosis, such as atherosclerosis by administering directly into an artery. In another particular embodiment, a composition comprising an effective amount of a compound of Formula (I), a compound of Formula (II), a senolytic agent or a combination thereof for treating a senescent-associated pulmonary disease or disorder may be administered by inhalation, intranasally, by intubation, or intracheally, for example, to provide the senolytic agent more directly to the affected pulmonary tissue. By way of another non-limiting example, a composition comprising an effective amount of a compound of Formula (I), a compound of Formula (II), a senolytic agent or a combination thereof may be delivered directly to the eye either by injection (e.g., intraocular or intravitreal) or by conjunctival application underneath an eyelid of a cream, ointment, gel, or eye drops. In more particular embodiments, a composition comprising an effective amount of a compound of Formula (I), a compound of Formula (II), a senolytic agent or a combination thereof may be formulated as a timed release (also called sustained release, controlled release) composition or may be administered as a bolus infusion.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments. Treatment may be before or after onset of the disease or disease symptoms.

Typical dosage levels can be determined and optimized using standard clinical techniques and will be dependent on the mode of administration.

### (e) Subject

A subject may be a rodent, a human, a livestock animal, a companion animal, or a zoological animal. In one embodiment, the subject may be a rodent (e.g., a mouse, a rat, a guinea pig, etc.). In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The human subject may be of any age. However, since senescent cells are normally associated with aging, a human subject may be an older human subject. In some embodiments, the human subject may be about 30, 35, 40, 45, 50, 55, 60, 65, 70, 75, 80, 85, 90, 95 years of age or older. In some preferred embodiments, the human subject is 30 years of age or older. In other preferred embodiments, the human subject is 40 years of age or older. In other preferred embodiments, the human subject is 45 years of age or older. In yet other preferred embodiments, the human subject is 50 years of age or older. In still other preferred embodiments, the human subject is 55 years of age or older. In other preferred embodiments, the human subject is 60 years of age or older. In yet other preferred embodiments, the human subject is 65 years of age or older. In still other preferred embodiments, the human subject is 70 years of age or older. In other preferred embodiments, the human subject is 75 years of age or older. In still other preferred embodiments, the human subject is 80 years of age or older. In yet other preferred embodiments, the human subject is 85 years of age or older. In still other preferred embodiments, the human subject is 90 years of age or older.

Additionally, a subject in need thereof may be a subject suffering from an age-related disease or condition as described below.

### (f) Aging and Age-related Diseases

It has been demonstrated that senescent cells drive age-related pathologies and that selective elimination of these cells can prevent or delay age-related deterioration. Thus, senescent cells may be therapeutic targets in the treatment of aging and age-related disease. As such, removal of senescent cells may delay tissue dysfunction and extend health span. Clearance of senescent cells is expected to improve tissue milieu, thereby improving the function of the remaining non-senescent cells.

The present disclosure provides a method for delaying at least one feature of aging in a subject, the method comprising administering a composition comprising a therapeutically effective amount of a compound of Formula (I) or a compound of Formula (II) to a subject. As used herein, “a feature of aging” may include, but is not limited to, systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, and cognitive impairment.

In an aspect, a composition of in the invention selectively kills senescent cells. In this way, targeting senescent cells during the course of aging may be a preventative strategy. Accordingly, administration of a composition comprising a therapeutically effective amount of a compound of Formula (I) or a compound of Formula (II) to a subject may prevent comorbidity and delay mortality in an older subject. Further, selective killing of senescent cells may boost the immune system, extend the health span, and improve the quality of life in a subject. Additionally, selective killing of senescent cells may delay sarcopenia. Sarcopenia is the degenerative loss of skeletal muscle mass, quality, and strength associated with aging. As such, a delay in sarcopenia may reduce frailty, reduce risk of falling, reduce fractures, and reduce functional disability in a subject. Furthermore, selective killing of senescent cells may delay aging of the skin. Aged skin has increased wrinkles, decreased immune barrier function and increased susceptibility to skin cancer and trauma. As such, selective killing of senescent cells may delay skin wrinkling, delay the onset of decreased immune barrier function and decrease susceptibility to skin cancer and trauma in a subject. Selective killing of senescent cells may also delay the onset of retinal atrophy and reduced lens transparency as measured by vision tests.

Methods of measuring aging are known in the art. For example, aging may be measured in the bone by incident non-vertebral fractures, incident hip fractures, incident total fractures, incident vertebral fractures, incident repeat fractures, functional recovery after fracture, bone mineral density decrease at the lumbar spine and hip, rate of knee buckling, NSAID use, number of joints with pain, and osteoarthritis. Aging may also be measured in the muscle by functional decline, rate of falls, reaction time and grip strength, muscle mass decrease at upper and lower extremities, and dual tasking 10-meter gait speed. Further, aging may be measured in the cardiovascular system by systolic and diastolic blood pressure change, incident hypertension, major cardiovascular events such as myocardial infarction, stroke, congestive heart disease, and cardiovascular mortality. Additionally, aging may be measured in the brain by cognitive decline, incident depression, and incident dementia. Also, aging may be measured in the immune system by rate of infection, rate of upper respiratory infections, rate of flu-like illness, incident severe infections that lead to hospital admission, incident cancer, rate of implant infections, and rate of gastrointestinal infections. Other indications of aging may include, but not limited to, decline in oral health, tooth loss, rate of GI symptoms, change in fasting glucose and/or insulin levels, body composition, decline in kidney function, quality of life, incident disability regarding activities of daily living, and incident nursing home admission. Methods of measuring skin aging are known in the art and may include trans-epidermal water loss (TEWL), skin hydration, skin elasticity, area ratio analysis of crow's feet, sensitivity, radiance, roughness, spots, laxity, skin tone homogeneity, softness, and relief (variations in depth).

The present disclosure also provides a method of treating an age-related disease or condition, the method comprising administering a composition comprising a therapeutically effective amount of a compound of Formula (I) or a compound of Formula (II) to a subject in need thereof, provided the age-related disease or condition is not cancer. As used herein, “age-related disease or condition” may include, but is not limited to, a degenerative disease or a function-decreasing disorder such as Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoporosis, osteoarthritis, type 2 diabetes, obesity, fat dysfunction, coronary artery disease, cerebrovascular disease, periodontal disease, cancer treatment-related disability such as atrophy and fibrosis in various tissues, brain and heart injury, and therapy-related myelodysplastic syndromes, and diseases associated with accelerated aging and/or defects in DNA damage repair and telomere maintenance such as progeroid syndromes (i.e., Hutchinson-Gilford progeria syndrome, Werner syndrome, Bloom syndrome, Rothmund-Thomson Syndrome, Cockayne syndrome, xeroderma pigmentosum, trichothiodystrophy, combined xeroderma pigmentosum-Cockayne syndrome, restrictive dermopathy), ataxia telangiectasia, Fanconi anemia, Friedreich's ataxia, dyskeratosis congenital, aplastic anemia, IPF, and others. Methods of diagnosing and identifying an age-related disease or condition are known in the art.

### Definitions

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

The compounds described herein have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated.

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

“Bcl-2” as used herein alone or as part of a group references to a member of the Bcl-2 family of proteins comprise the following Bcl-x_{L}, MCL-1, Bcl-W, BFL-1/A1, Bcl-B, BAX, BAK, and BOK.

“Alkyl” as used herein alone or as part of a group refers to saturated monovalent hydrocarbon radicals having straight or branched hydrocarbon chains or, in the event that at least 3 carbon atoms are present, cyclic hydrocarbons or combinations thereof and contains 1 to 20 carbon atoms (C₁-C₂₀alkyl), suitably 1 to 10 carbon atoms (C₁-C₁₀ alkyl), preferably 1 to 8 carbon atoms (C₁-C₈alkyl), more preferably 1 to 6 carbon atoms (C₁-C₄alkyl), and even more preferably 1 to 4 carbon atoms (C₁-C₄alkyl). Examples of alkyl radicals include methyl, ethyl, propyl, isopropyl, n-butyl, isobutyl, sec-butyl, tert-butyl, pentyl, isoamyl, hexyl, cyclopropyl, cyclobutyl, cyclopentyl, cyclohexyl and the like.

“Aryl” as used herein, alone or as part of a group, includes an organic radical derived from an aromatic hydrocarbon by removal of one hydrogen, and includes monocyclic and polycyclic radicals, such as phenyl, biphenyl, naphthyl.

“Cycloalkyl” as used herein, alone or in combination, means a saturated or partially saturated monocyclic, bicyclic or tricyclic alkyl radical wherein each cyclic moiety contains from about 3 to about 8 carbon atoms, more preferably from about 3 to about 6 carbon atoms. Examples of such cycloalkyl radicals include cyclopropyl, cyclobutyl, cyclopentyl, cyclohexyl and the like.

“Heteroatom” means an atom other than carbon e.g., in the ring of a heterocyclic group or the chain of a heterogeneous group. Preferably, heteroatoms are selected from the group consisting of sulfur, phosphorous, nitrogen, and oxygen atoms. Groups containing more than one heteroatom may contain different heteroatoms.

“Heteroaryl” as used herein, along or in combination, includes an organic radical derived from an aromatic hydrocarbon by removal of one hydrogen and includes at least one heteroatom. Examples of heteroaryl includes pyrrole, thiophene, furan, indole, pyrazine, pyridine, triazole, imidazole, thiazole, oxazole and the like.

“Substituted” means that one or more of the hydrogen atoms bonded to carbon atoms in the chain or ring have been replaced with other substituents. Suitable substituents include monovalent hydrocarbon groups including alkyl groups such as methyl groups and monovalent heterogeneous groups including alkoxy groups such as methoxy groups. “Unsubstituted” means that the carbon chain or ring contains no other substituents other than carbon and hydrogen.

“Branched” means that the carbon chain is not simply a linear chain. “Unbranched” means that the carbon chain is a linear carbon chain.

“Heteroatom” means an atom other than carbon e.g., in the ring of a heterocyclic group or the chain of a heterogeneous group. Preferably, heteroatoms are selected from the group consisting of sulfur, phosphorous, nitrogen and oxygen atoms. Groups containing more than one heteroatom may contain different heteroatoms.

“Heterocyclic group” means a saturated or unsaturated ring structure containing carbon atoms and 1 or more heteroatoms in the ring. Heterocyclic groups are not aromatic. Heterocyclic groups are monocyclic or polycyclic. Polycyclic heteroaromatic groups can be fused, spiro, or bridged ring systems. Monocyclic heterocyclic groups contain 4 to 10 member atoms (i.e., including both carbon atoms and at least 1 heteroatom), suitably 4 to 7, and more suitably 5 to 6 in the ring. Bicyclic heterocyclic groups contain 8 to 18 member atoms, suitably 9 or 10 in the rings.

“Isomer”, “isomeric form”, “stereochemically isomeric forms” or “stereolsomeric forms”, as used herein, defines all possible isomeric as well as conformational forms, made up of the same atoms bonded by the same sequence of bonds but having different three-dimensional structures which are not interchangeable, which compounds or intermediates obtained during said process may possess. Unless otherwise mentioned or indicated, the chemical designation of a compound encompasses the mixture of all possible stereochemically isomeric forms which said compound may possess. Said mixture may contain all diastereoisomers, epimers, enantiomers and/or conformers of the basic molecular structure of said compound. More in particular, stereogenic centers may have the R- or S-configuration, diastereoisomers may have a syn- or anti-configuration, substituents on bivalent cyclic saturated radicals may have either the cis- or trans-configuration and alkenyl radicals may have the E- or Z-configuration. All stereochemically isomeric forms of said compound both in pure form or in admixture with each other are intended to be embraced within the scope of the present invention.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The compounds of the present invention may be prepared in a number of ways well known to one skilled in the art of organic synthesis. More specifically, the novel compounds of this invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, are chosen to be the conditions standard for that reaction. It is understood by one skilled in the art of organic synthesis that the functionality present on various portions of the molecule must be compatible with the reagents and reactions proposed. Such restrictions to the substituents, which are not compatible with the reaction conditions, will be apparent to one skilled in the art and alternate methods must then be used. Unless otherwise stated, the starting materials for the examples contained herein are either commercially available or are readily prepared by standard methods from known materials. The compounds of Formula (I) or Formula (II) may be synthesized through standard organic chemistry methodology and purification known to those trained in the art of organic synthesis by using commercially available starting materials and reagents.

The following abbreviations are used: s: singlet; d: doublet; t: triplet; q: quartet; m: multiplet; dd: doublet of doublet; dt: doublet of triplet: dq: doublet of quartet; br: broad; AcOH=acetic acid; DCM=dichloromethane; DIPEA=N,N-diisopropylethylamine; DMAP=4-dimethylaminopyridine; DMF=N,N-dimethylformamide; DMSO=dimethylsulfoxide; EDCI=1-ethyl-3-(3-dimethylaminopropyl)carbodiimide; EDTA=ethylenediaminetetraacetic acid; EtOAc=ethyl acetate; FBS=fetal bovine serum; HATU=1-[Bis(dimethylamino)methylene]-1H-1,2,3-triazolo[4,5-b]pyridinium 3-oxid hexafluorophosphate; HCl=hydrochloric acid; MOMCI=chloromethyl methyl ether; PBS=phosphate buffered saline; TBAF=tetra-n-butylammonium fluoride; TBSCI=tert-butyldimethylchlorosilane; TBS-T=tris-buffered saline; TEA=triethylamine; THF=tetrahydrofuran; and TFA=trifluoroacetic acid.

### Example 1: Synthesis of XZ-13906

### Preparation of (4-bromo-2-fluorophenoxy)(tert-butyl)dimethylsilane (2)

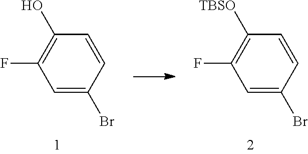 [SMILES: Oc1ccc(Br)cc1F ; CC(C)(C)[Si](C)(C)Oc1ccc(Br)cc1F]

4-Bromo-2-fluorophenol (1.0 g, 5.24 mmol), TBSCl (1.03 g, 6.83 mmol) and imidazole (713 mg, 10.48 mmol) were dissolved in 20 mL DMF and the mixture was stirred at room temperature for 16 hours. Water was added to the reaction mixture and extracted with EtOAc. The combined organic phases were washed with water×1, brine×1, dried over Na₂SO₄, filtered and evaporated to dryness. The resulting oil was further purified by column chromatography to afford 1.60 g compound 2 as colorless oil. Yield 100%. ¹H NMR (400 MHz, CDCl₃) δ 7.22 (dd, J=10.1, 2.4 Hz, 1H), 7.15-7.07 (m, 1H), 6.79 (t, J=8.7 Hz, 1H), 1.00 (s, 9H), 0.19 (d, J=0.9 Hz, 6H) ppm.

### Preparation of tert-butyl 4-(prop-2-ynyl)piperazine-1-carboxylate (4)

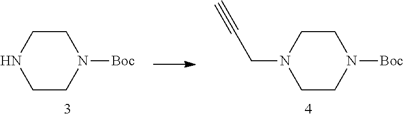 [SMILES: CC(C)(C)OC(=O)N1CCNCC1 ; C#CCN1CCN(C(=O)OC(C)(C)C)CC1]

1-Boc-Piperazine 3 (1.0 g, 5.38 mmol), 80% 3-bromoprop-1-yne toluene solution (900 μL, 8.07 mmol), and DIPEA (1.78 mL, 10.76 mmol) were dissolved in 25 mL DCM and the mixture was stirred at room temperature for 16 hours. Water was added to the reaction mixture and the aqueous phase was extracted with DCM. The combined organic phases were washed with brine, dried over Na₂SO₄, filtered and evaporated to dryness. The resulting oil was further purified by column chromatography to afford 1.13 g compound 4. Yield 94%. ¹H NMR (400 MHz, CDCl₃) δ 3.54-3.42 (m, 4H), 3.33 (d, J=2.4 Hz, 2H), 2.57-2.46 (m, 4H), 2.26 (t, J=2.4 Hz, 1H), 1.46 (s, 9H) ppm.

### Preparation of tert-butyl 4-(3-(4-(tert-butyldimethylsilyloxy)-3-fluorophenyl)prop-2-ynyl)piperazine-1-carboxylate (5)

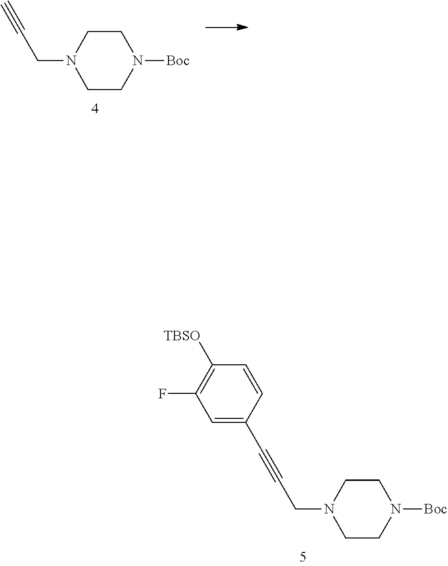 [SMILES: C#CCN1CCN(C(=O)OC(C)(C)C)CC1 ; CC(C)(C)OC(=O)N1CCN(CC#Cc2ccc(O[Si](C)(C)C(C)(C)C)c(F)c2)CC1]

A mixture of compound 2 (612 mg, 2 mmol), compound 4 (448 mg, 2 mmol), Pd(PPh₃)₄ (68 mg, 0.06 mmol), CuI (12 mg, 0.06 mmol), and TEA (700 μL, 4.2 mmol) were stirred in 15 mL DMF at 100° C. under Argon for 20 hours. Water was added to the reaction mixture and extracted with EtOAc. The combined organic phases were washed with water×1, brine×1, dried over Na₂SO₄, filtered and evaporated to dryness. The resulting oil was further purified by column chromatography to afford 220 mg compound 5. Yield 25%. ¹H NMR (400 MHz, CDCl₃) δ 7.13 (dd, J=11.1, 2.0 Hz, 1H), 7.10-7.04 (m, 1H), 6.83 (t, J=8.5 Hz, 1H), 3.58-3.41 (m, 6H), 2.68-2.50 (m, 4H), 1.47 (s, 9H), 1.00 (s, 9H), 0.19 (d, J=0.9 Hz, 6H) ppm.

### Preparation of tert-butyl 4-(3-(3-fluoro-4-hydroxyphenyl)prop-2-ynyl)piperazine-1-carboxylate (6)

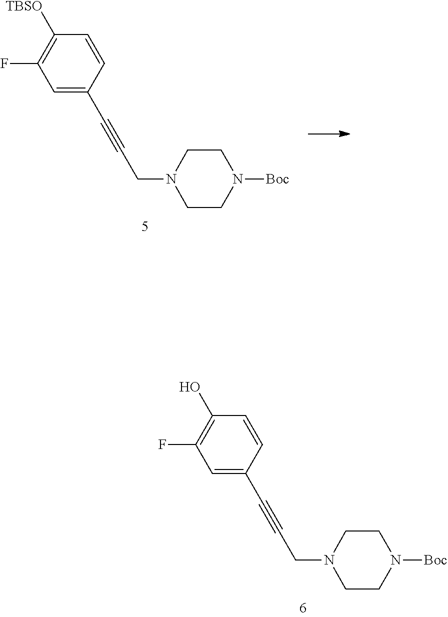 [SMILES: CC(C)(C)OC(=O)N1CCN(CC#Cc2ccc(O[Si](C)(C)C(C)(C)C)c(F)c2)CC1 ; CC(C)(C)OC(=O)N1CCN(CC#Cc2ccc(O)c(F)c2)CC1]

To a solution of compound 5 (180 mg, 0.4 mmol) in 5 mL THF was added 0.8 mL TBAF solution (1.0 M in THF) dropwise. After 30 minutes, water was added to the reaction mixture and extracted with EtOAc. The organic phase was washed with saturated NH₄Cl (aq)×1, brine×1, dried over Na₂SO₄, filtered and evaporated to dryness. The resulting mixture was further purified by column chromatography to afford 126 mg compound 6 as brown solid. Yield 94%. ¹H NMR (400 MHz, CDCl₃) δ 7.09-7.00 (m, 2H), 6.89 (t, J=8.8 Hz, 1H), 3.59-3.45 (m, 6H), 2.70-2.54 (m, 4H), 2.08 (s, 1H), 1.47 (s, 9H) ppm.

### Preparation of 2-(2,6-dioxopiperidin-3-yl)-4-(2-(2-(2-(prop-2-ynyloxy)ethoxy)ethoxy) ethylamino) isoindoline-1,3-dione (9)

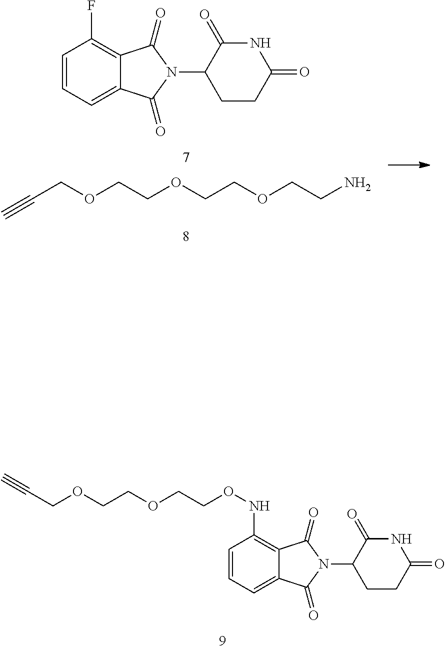 [SMILES: O=C1CCC(N2C(=O)c3cccc(F)c3C2=O)C(=O)N1 ; C#CCOCCOCCOCCN ; C#CCOCCOCCONc1cccc2c1C(=O)N(C1CCC(=O)NC1=O)C2=O]

2-(2,6-Dioxopiperidin-3-yl)-4-fluoroisoindoline-1,3-dione (7) was synthesized according to reported method with minor modifications (Chem. Biol. 22:755-763, 2015). Compound 7 (100 mg, 0.36 mmol), amine 8 (68 mg, 0.36 mmol), and DIPEA (120 μL, 0.72 mmol) in 4 mL DMF were stirred at 90° C. for 16 hours. Water was added to the reaction mixture and extracted with EtOAc. The organic phase was washed with water×1, brine×1, dried over Na₂SO₄, filtered and evaporated to dryness. The resulting mixture was further purified by column chromatography to afford 95 mg compound 9 as a green solid. Yield 59%. ¹H NMR (400 MHz, CDCl₃) δ 8.02 (s, 1H), 7.64-7.34 (m, 1H), 7.10 (d, J=7.1 Hz, 1H), 6.93 (d, J=8.6 Hz, 1H), 6.67-6.11 (m, 1H), 4.91 (dd, J=12.1, 5.3 Hz, 1H), 4.20 (d, J=2.2 Hz, 2H), 3.83-3.60 (m, 10H), 3.55-3.40 (m, 2H), 2.99-2.60 (m, 3H), 2.43 (t, J=2.1 Hz, 1H), 2.21-2.03 (m, 1H) ppm.

### Preparation of 2-(8-(benzo[d]thiazol-2-ylcarbamoyl)-3,4-dihydroisoquinolin-2(1H)-yl)-5-(3-(4-(3-(4-(tert-butoxycarbonyl)piperazin-1-yl)prop-1-ynyl)-2-fluorophenoxy)propyl)thiazole-4-carboxylic acid (11)

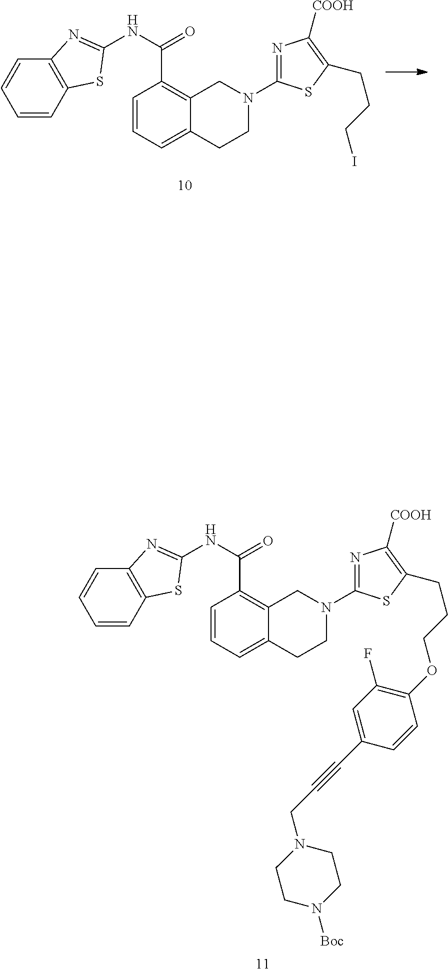 [SMILES: O=C(Nc1nc2ccccc2s1)c1cccc2c1CN(c1nc(C(=O)O)c(CCCI)s1)CC2 ; CC(C)(C)OC(=O)N1CCN(CC#Cc2ccc(OCCCc3sc(N4CCc5cccc(C(=O)Nc6nc7ccccc7s6)c5C4)nc3C(=O)O)c(F)c2)CC1]

Compound 10 was synthesized according to reported method with minor modifications (ACS Med Chem Lett. 5:1088-1093, 2014). Compound 6 (200 mg, 0.60 mmol) in 5 mL DMF was cooled to 0° C. and 40 mg 95% NaH was added to the solution. The resulting reaction mixture was stirred for 10 min before the addition of compound 10 (250 mg, 0.40 mmol) in 5 mL THF. The mixture was stirred at room temperature for 3 hours and quenched by adding 1 mL water. The pH was adjusted to 5 using 1N HCl (aq) and the resulted solution was extracted with EtOAc. The organic phase was washed with water×1, brine×1, dried over Na₂SO₄, filtered and evaporated to dryness. The resulting mixture was further purified by column chromatography to afford 130 mg compound 11. Yield 41%. ¹H NMR (400 MHz, CDCl₃) δ 7.90-7.76 (m, 1H), 7.69-7.59 (m, 1H), 7.54-7.41 (m, 1H), 7.36-7.29 (m, 4H), 7.14-7.05 (m, 2H), 6.80 (t, J=8.5 Hz, 1H), 4.93 (s, 2H), 4.00 (t, J=6.2 Hz, 2H), 3.79-3.65 (m, 2H), 3.57-3.49 (m, 6H), 3.28 (t, J=7.3 Hz, 2H), 3.09-2.88 (m, 2H), 2.74-2.46 (m, 4H), 2.30-2.06 (m, 2H), 1.46 (s, 9H) ppm.

### Preparation of 2-(8-(benzo[d]thiazol-2-ylcarbamoyl)-3,4-dihydroisoquinolin-2(1H)-yl)-5-(3-(2-fluoro-4-(3-(piperazin-1-yl)prop-1-ynyl)phenoxy)propyl)thiazole-4-carboxylic acid (12)

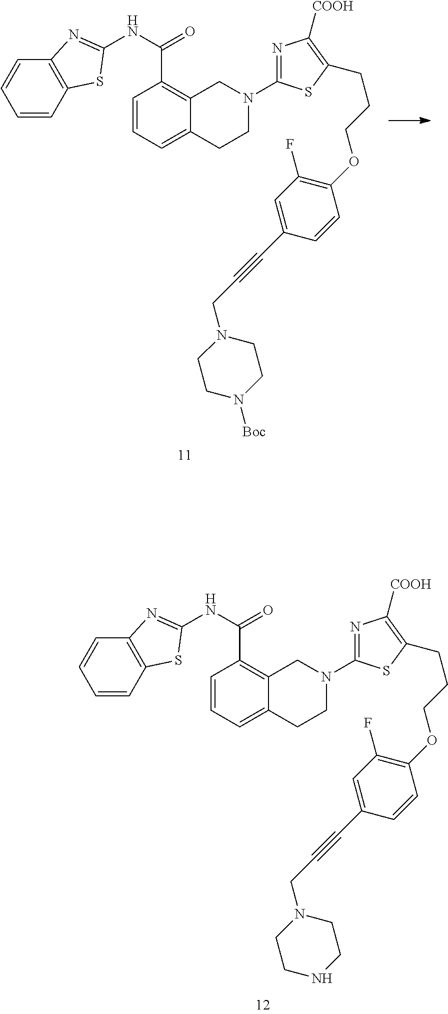 [SMILES: CC(C)(C)OC(=O)N1CCN(CC#Cc2ccc(OCCCc3sc(N4CCc5cccc(C(=O)Nc6nc7ccccc7s6)c5C4)nc3C(=O)O)c(F)c2)CC1 ; O=C(Nc1nc2ccccc2s1)c1cccc2c1CN(c1nc(C(=O)O)c(CCCOc3ccc(C#CCN4CCNCC4)cc3F)s1)CC2]

A mixture of compound 11 (130 mg) and TFA (1 mL) in 3 mL DCM was stirred at room temperature for 1 hour. The solvent was removed under reduced pressure and the crude product was crystallized in Et₂O and MeOH to give 110 mg compound 12 as a pale yellow solid. ¹H NMR (400 MHz, CD₃OD) δ 7.93 (d, J=7.7 Hz, 1H), 7.78 (d, J=7.7 Hz, 1H), 7.63 (d, J=7.2 Hz, 1H), 7.52-7.28 (m, 5H), 7.18-7.07 (m, 2H), 6.99 (t, J=8.7 Hz, 1H), 4.91 (s, 2H), 4.07 (t, J=6.1 Hz, 2H), 3.89-3.77 (m, 2H), 3.62 (s, 2H), 3.28-3.20 (m, 6H), 3.09-3.05 (m, 2H), 2.93-2.80 (m, 4H), 2.20-2.07 (m, 2H) ppm.

### Preparation of 5-(3-(4-(3-(4-(4-azidobutanoyl)piperazin-1-yl)prop-1-ynyl)-2-fluorophenoxy)propyl)-2-(8-(benzo[d]thiazol-2-ylcarbamoyl)-3,4-dihydroisoquinolin-2(1H)-yl)thiazole-4-carboxylic acid (13)

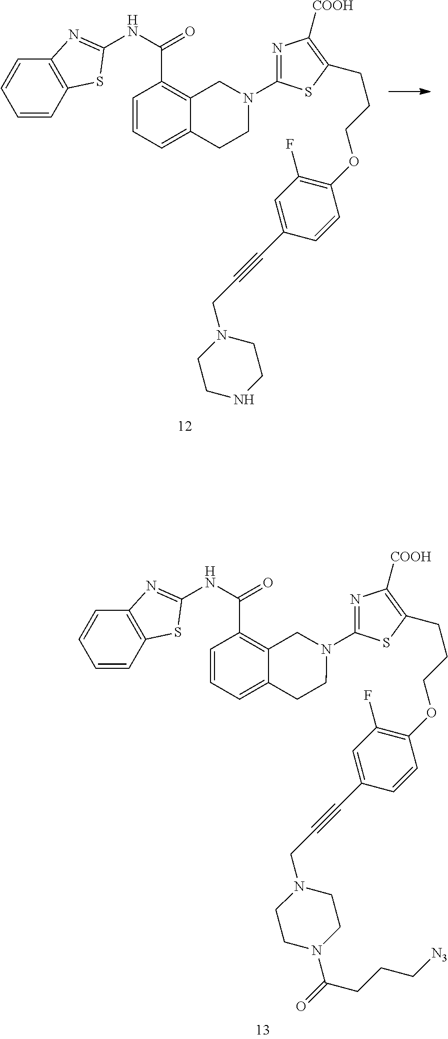 [SMILES: O=C(Nc1nc2ccccc2s1)c1cccc2c1CN(c1nc(C(=O)O)c(CCCOc3ccc(C#CCN4CCNCC4)cc3F)s1)CC2 ; [N-]=[N+]=NCCCC(=O)N1CCN(CC#Cc2ccc(OCCCc3sc(N4CCc5cccc(C(=O)Nc6nc7ccccc7s6)c5C4)nc3C(=O)O)c(F)c2)CC1]

Compound 12 (100 mg) and TEA (157 μL) in 4 mL DCM was stirred at room temperature. 4-Azidobutanoyl chloride (16.4 mg) in 660 μL DCM was then added dropwise to the mixture. The reaction was quenched after stirred for 10 minutes by adding 1 mL MeOH. DCM was added and the mixture was washed water×1, brine×1, dried over Na₂SO₄, filtered and evaporated to dryness. The crude product was crystallized in MeOH to give 85 mg pale yellow solid. ¹H NMR (400 MHz, CDCl₃) δ 7.86 (d, J=7.8 Hz, 1H), 7.69-7.59 (m, 2H), 7.44-7.29 (m, 4H), 7.15-7.05 (m, 2H), 6.82 (t, J=8.7 Hz, 1H), 4.95 (s, 2H), 4.04 (t, J=6.3 Hz, 2H), 3.81-3.64 (m, 6H), 3.44-3.24 (m, 6H), 3.06 (t, J=5.9 Hz, 2H), 2.89-2.58 (m, 4H), 2.42 (t, J=7.2 Hz, 2H), 2.22-2.11 (m, 2H), 1.99-1.87 (m, 2H) ppm.

### Preparation of 2-(8-(benzo[d]thiazol-2-ylcarbamoyl)-3,4-dihydroisoquinolin-2(1H)-yl)-5-(3-(4-(3-(4-(4-(4-((2-(2-(2-(2-(2,6-dioxopiperidin-3-yl)-1,3-dioxoisoindolin-4-ylamino)ethoxy)ethoxy)ethoxy)methyl)-1H-1,2,3-triazol-1-yl)butanoyl)piperazin-1-yl)prop-1-ynyl)-2-fluorophenoxy)propyl)thiazole-4-carboxylic acid (XZ-13906)

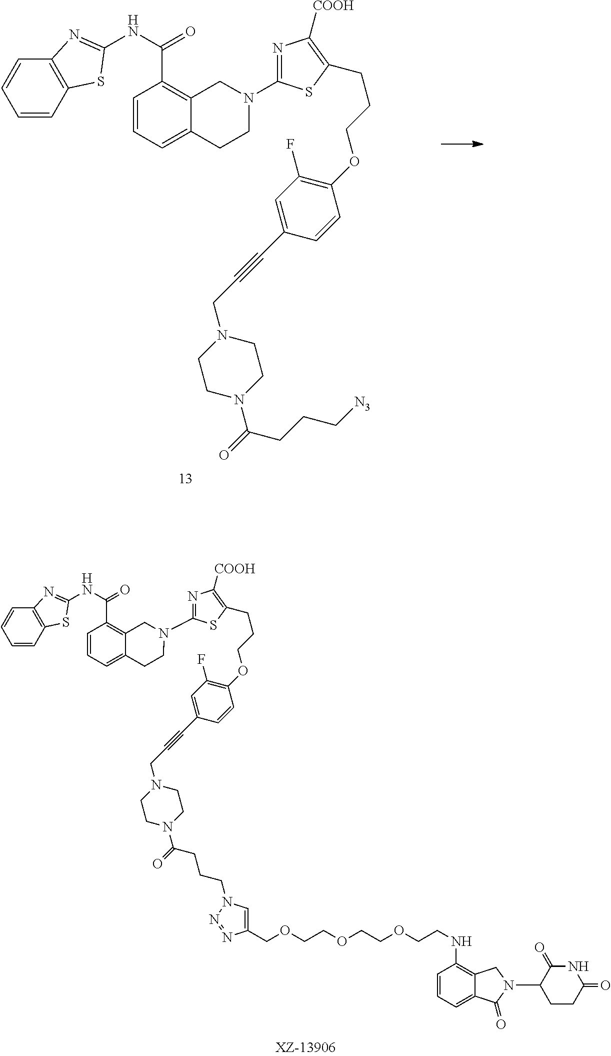 [SMILES: [N-]=[N+]=NCCCC(=O)N1CCN(CC#Cc2ccc(OCCCc3sc(N4CCc5cccc(C(=O)Nc6nc7ccccc7s6)c5C4)nc3C(=O)O)c(F)c2)CC1 ; O=C1CCC(N2Cc3c(NCCOCCOCCOCc4cn(CCCC(=O)N5CCN(CC#Cc6ccc(OCCCc7sc(N8CCc9cccc(C(=O)Nc%10nc%11ccccc%11s%10)c9C8)nc7C(=O)O)c(F)c6)CC5)nn4)cccc3C2=O)C(=O)N1]

To a mixture of compound 13 (18 mg), compound 9 (10 mg) in 1 mL t-BuOH under Argon was added CuSO₄.5H₂O (1.0 mg) and sodium ascorbate (0.8 mg) in 0.2 mL water. The mixture was stirred at 65° C. for 16 hours and extracted with DCM. The organic phase was washed brine×1, dried over Na₂SO₄, filtered and evaporated to dryness. The crude product was purified using reverse phase preparative HPLC to give 4.0 mg pure product as yellow solid. ¹H NMR (400 MHz, CDCl₃) δ 10.17 (s, 1H), 7.92-7.82 (m, 2H), 7.71 (d, J=6.9 Hz, 1H), 7.53 (s, 1H), 7.50-7.43 (m, 2H), 7.37 (t, J=7.5 Hz, 1H), 7.33-7.27 (m, 2H), 7.15-7.02 (m, 3H), 6.87 (d, J=8.6 Hz, 1H), 6.79 (t, J=8.4 Hz, 1H), 6.48 (br s, 1H), 4.99-4.83 (m, 3H), 4.70-4.53 (m, 2H), 4.37 (t, J=5.9 Hz, 2H), 4.11-3.94 (m, 4H), 3.82-3.56 (m, 16H), 3.42 (t, J=4.8 Hz, 2H), 3.35-3.06 (m, 6H), 3.02 (t, J=5.7 Hz, 2H), 2.89-2.67 (m, 3H), 2.33-2.05 (m, 7H) ppm.

### Example 2: Synthesis of XZ-13942

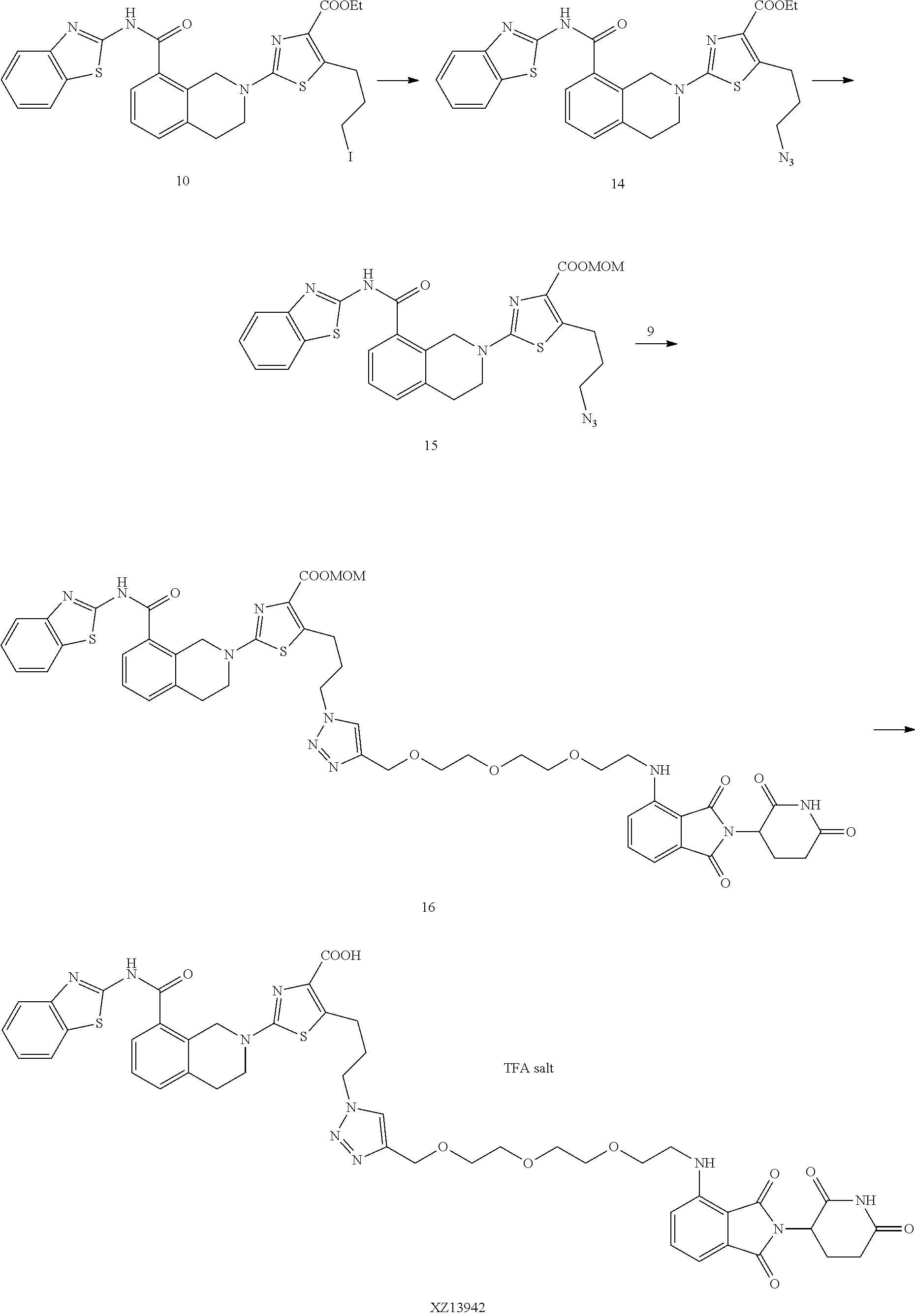

### Preparation of ethyl 5-(3-azidopropyl)-2-(8-(benzo[d]thiazol-2-ylcarbamoyl)-3,4-dihydroisoquinolin-2(1H)-yl)thiazole-4-carboxylate (14)

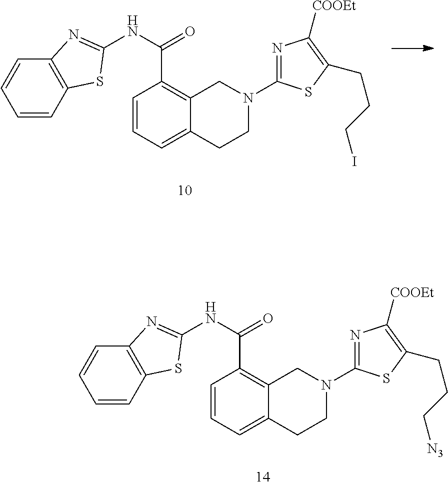 [SMILES: CCOC(=O)c1nc(N2CCc3cccc(C(=O)Nc4nc5ccccc5s4)c3C2)sc1CCCI ; CCOC(=O)c1nc(N2CCc3cccc(C(=O)Nc4nc5ccccc5s4)c3C2)sc1CCCN=[N+]=[N-]]

Compound 10 (100 mg) and NaN₃ (13 mg) were stirred in 5 mL DMSO at 45° C. overnight. The mixture was poured into water and extracted with EtOAc. The organic phase was washed with water×1, brine×1, dried over Na₂SO₄, filtered and evaporated to dryness to give 85 mg pure product as white solid. Yield 98%. ¹H NMR (400 MHz, CDCl₃) δ 7.82 (d, J=6.7 Hz, 1H), 7.54 (d, J=7.6 Hz, 1H), 7.35-7.26 (m, 4H), 7.18 (t, J=7.6 Hz, 1H), 4.87 (s, 2H), 4.28 (q, J=7.1 Hz, 2H), 3.90-3.79 (m, 2H), 3.31 (t, J=6.6 Hz, 2H), 3.11 (t, J=7.4 Hz, 2H), 3.03-2.92 (m, 2H), 1.98-1.82 (m, 2H), 1.31 (t, J=7.1 Hz, 3H) ppm.

### Preparation of methoxymethyl 5-(3-azidopropyl)-2-(8-(benzo[d]thiazol-2-ylcarbamoyl)-3,4-dihydroisoquinolin-2(1H)-yl)thiazole-4-carboxylate (15)

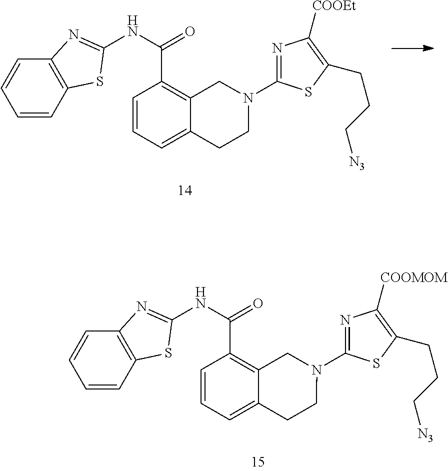

Compound 14 (85 mg) and NaOH (26.5 mg) were stirred in a mixture of ethanol and water at 50° C. for 5 hours. The mixture was cooled to room temperature and neutralized with 1N HCl (aq.). The precipitated solid was collected and dissolved in 4 mL DMF. Then Na₂CO₃ (17 mg) and MOMCl (12 mg) was added into the mixture. After 16 hours, the mixture was poured into water and extracted with EtOAc. The organic phase was washed with water×1, brine×1, dried over Na₂SO₄, filtered and evaporated to dryness. The resulting mixture was purified via column chromatography using EtOAc and hexanes as eluents to afford 53 mg compound 15. Yield 61%. ¹H NMR (400 MHz, CDCl₃) δ 7.88 (d, J=7.7 Hz, 1H), 7.75 (d, J=7.1 Hz, 1H), 7.68 (d, J=7.9 Hz, 1H), 7.51-7.29 (m, 5H), 5.40 (s, 2H), 4.94 (s, 2H), 3.90 (t, J=6.1 Hz, 2H), 3.50 (s, 3H), 3.35 (t, J=6.8 Hz, 2H), 3.17 (t, J=7.5 Hz, 2H), 3.08 (t, J=6.1 Hz, 2H), 1.99-1.88 (m, 2H) ppm.

### Preparation of 2-(8-(benzo[d]thiazol-2-ylcarbamoyl)-3,4-dihydroisoquinolin-2(1H)-yl)-5-(3-(4-((2-(2-(2-((2-(2,6-dioxopiperidin-3-yl)-1,3-dioxoisoindolin-4-14)amino)ethoxy)ethoxy)ethoxy)methyl)-1H-1,2,3-triazol-1-yl)propyl)thiazole-4-carboxylic acid (XZ13942)

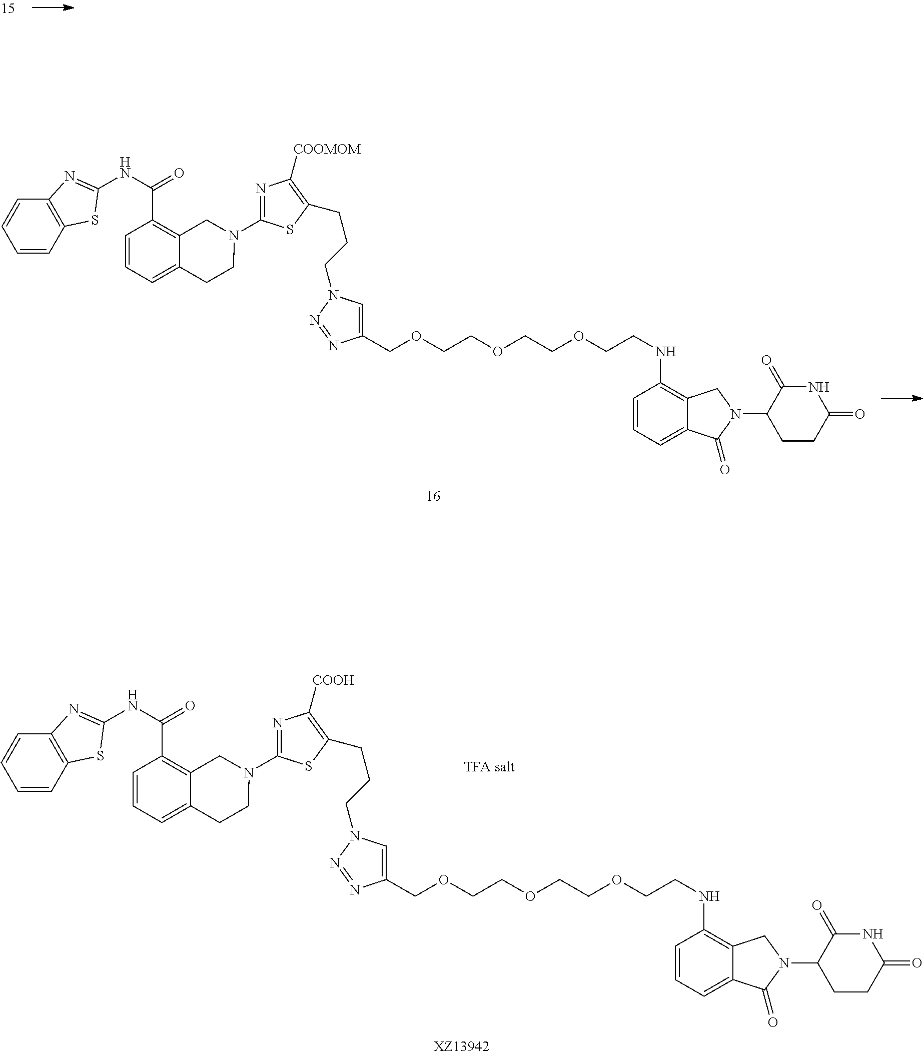

To a mixture of compound 15 (10 mg), compound 9 (8.7 mg) in 2 mL t-BuOH-THF (1:1, v/v) under argon was added CuSO₄.5H₂O (0.9 mg) and sodium ascorbate (0.7 mg) in 0.4 mL water. The mixture was stirred at 60° C. for 16 hours and extracted with DCM. The organic phase was washed with brine×1, dried over Na₂SO₄, filtered and evaporated to dryness. The crude product was purified via column chromatography using DCM and methanol as eluents to afford 14 mg compound 16. Yield 78%. Compound 16 (9.0 mg) and 0.1 mL TFA was stirred in 3 mL DCM for 1 hour. The solvent was removed under reduced pressure. Then Et₂O was added into the residue and the precipitated solid was collected to afford 8.4 mg pure XZ13942. Yield 79%. ¹H NMR (400 MHz, CDCl₃) δ 10.03 (s, 1H), 7.94 (d, J=6.4 Hz, 1H), 7.87 (d, J=7.7 Hz, 1H), 7.77 (d, J=6.7 Hz, 1H), 7.64-7.29 (m, 6H), 7.07 (d, J=7.0 Hz, 1H), 6.88 (d, J=8.5 Hz, 1H), 5.03-4.86 (m, 3H), 4.75-4.55 (m, 2H), 4.46-4.30 (m, 2H), 3.86-3.58 (m, 12H), 3.46-3.33 (m, 2H), 3.25-2.98 (m, 4H), 2.92-2.68 (m, 3H), 2.28-2.05 (m, 3H) ppm.

### Example 3: Synthesis of XZ-14424

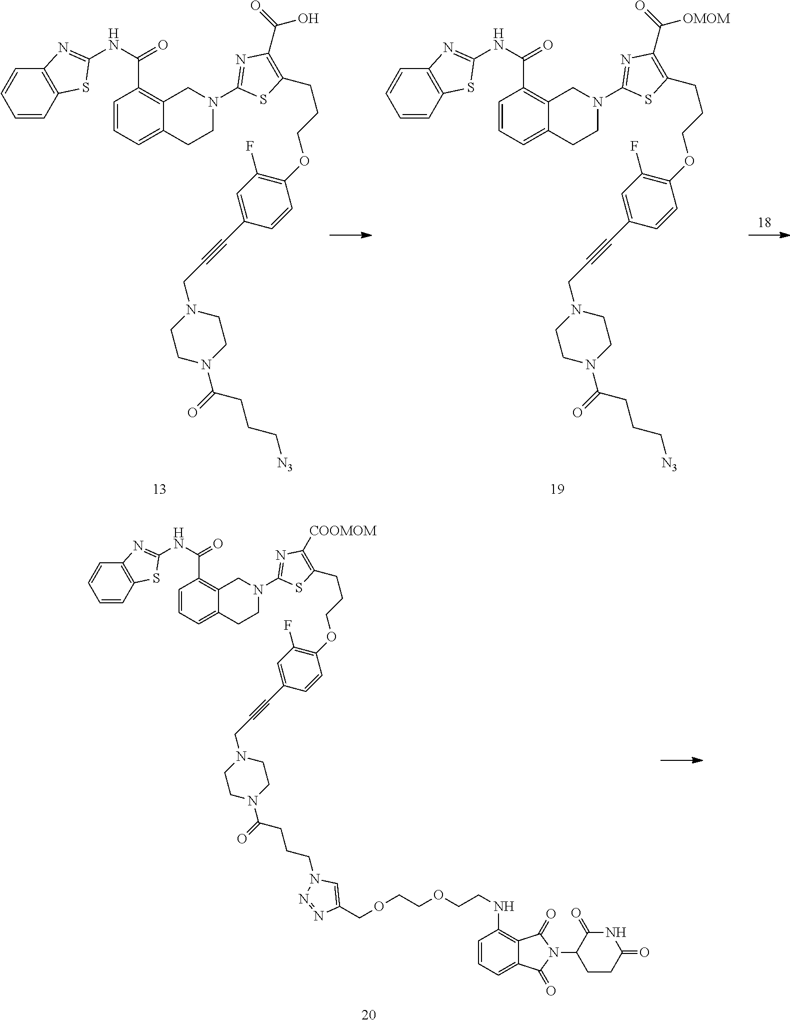

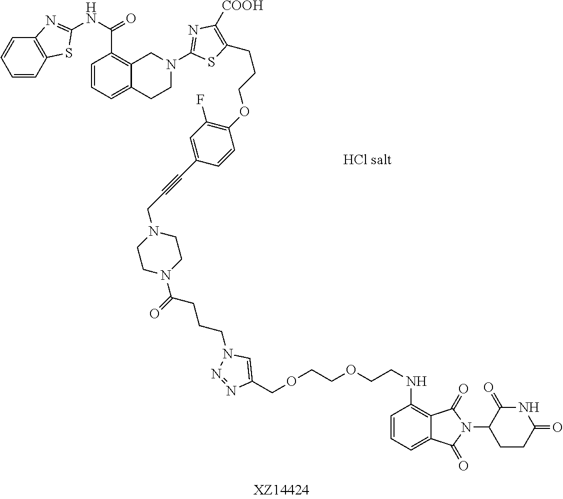 [SMILES: O=C1CCC(N2C(=O)c3cccc(NCCOCCOCc4cn(CCCC(=O)N5CCN(CC#Cc6ccc(OCCCc7sc(N8CCc9cccc(C(=O)Nc%10nc%11ccccc%11s%10)c9C8)nc7C(=O)O)c(F)c6)CC5)nn4)c3C2=O)C(=O)N1 ; "HCl salt"]

### Preparation of 2-(2,6-dioxopiperidin-3-yl)-4-((2-(2-(prop-2-yn-1-yloxy)ethoxy)ethyl)amino) isoindoline-1,3-dione (18)

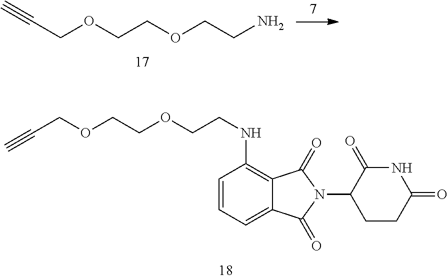

Compound 7 (107 mg), amine 17 (84 mg), and DIPEA (193 μL) in 5 mL DMF were stirred at 85° C. for 16 hours. Water was added to the reaction mixture and extracted with EtOAc. The organic phase was washed with water×1, brine×1, dried over Na₂SO₄, filtered and evaporated to dryness. The resulting mixture was purified by column chromatography using EtOAc and hexanes as eluents to afford 50 mg compound 18 as a green solid. Yield 32%. ¹H NMR (400 MHz, CDCl₃) δ 7.98 (s, 1H), 7.62-7.35 (m, 1H), 7.11 (d, J=7.1 Hz, 1H), 6.93 (d, J=8.5 Hz, 1H), 4.92 (dd, J=11.9, 5.3 Hz, 1H), 4.21 (d, J=2.3 Hz, 2H), 3.78-3.66 (m, 6H), 3.49 (t, J=5.4 Hz, 2H), 2.93-2.68 (m, 3H), 2.48-2.41 (m, 1H), 2.18-2.09 (m, 1H) ppm.

### Preparation of methoxymethyl 5-(3-(4-(3-(4-(4-azidobutanoyl)piperazin-1-yl)prop-1-yn-1-yl)-2-fluorophenoxy)propyl)-2-(8-(benzo[d]thiazol-2-ylcarbamoyl)-3,4-dihydroisoquinolin-2(1H)-yl)thiazole-4-carboxylate (19)

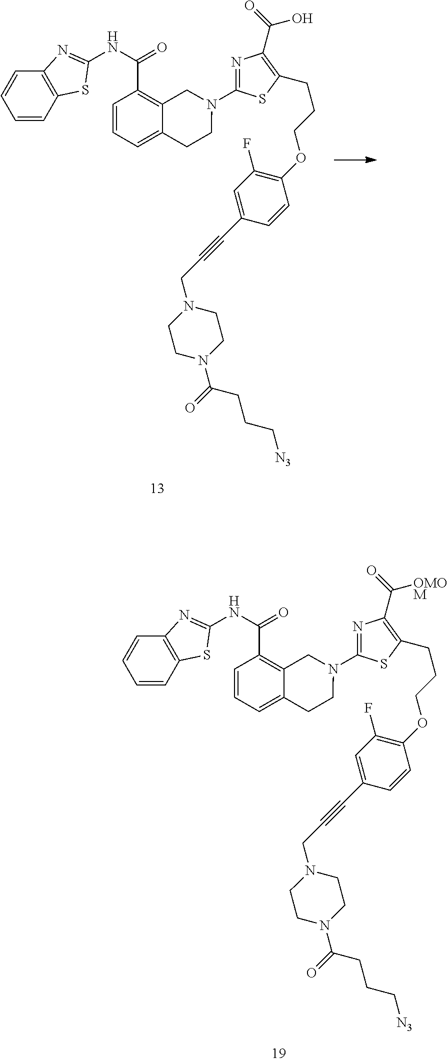

Compound 13 (26 mg), Na₂CO₃ (4.1 mg) and MOMCl (2.8 mg) were stirred in 2 mL DMF for 24 hours. Then it was poured into water and extracted with EtOAc. The organic phase was washed with water×1, brine×1, dried over Na₂SO₄, filtered and evaporated to dryness. The resulting mixture was purified via column chromatography using DCM and methanol as eluents to afford 15 mg compound 19. Yield 58%. ¹H NMR (400 MHz, CDCl₃) δ 7.90-7.77 (m, 1H), 7.54 (d, J=7.6 Hz, 1H), 7.37-7.25 (m, 4H), 7.18 (t, J=7.6 Hz, 1H), 7.12-7.04 (m, 2H), 6.81 (t, J=8.4 Hz, 1H), 5.34 (s, 2H), 4.88 (s, 2H), 4.03 (t, J=6.2 Hz, 2H), 3.81 (t, J=6.0 Hz, 2H), 3.76-3.64 (m, 2H), 3.63-3.49 (m, 4H), 3.44 (s, 3H), 3.36 (t, J=6.3 Hz, 2H), 3.25 (t, J=7.4 Hz, 2H), 3.00 (t, J=5.9 Hz, 2H), 2.71-2.53 (m, 4H), 2.40 (t, J=7.2 Hz, 2H), 2.23-2.06 (m, 2H), 1.98-1.84 (m, 2H) ppm.

### Preparation of 2-(8-(benzo[d]thiazol-2-ylcarbamoyl)-3,4-dihydroisoquinolin-2(1H)-yl)-5-(3-(4-(3-(4-(4-(4-((2-(2-((2-(2,6-dioxopiperidin-3-yl)-1,3-dioxoisoindolin-4-yl)amino)ethoxy)ethoxy)methyl)-1H-1,2,3-triazol-1-yl)butanoyl)piperazin-1-yl)prop-1-yn-1-yl)-2-fluorophenoxy)propyl)thiazole-4-carboxylic acid (XZ14424)

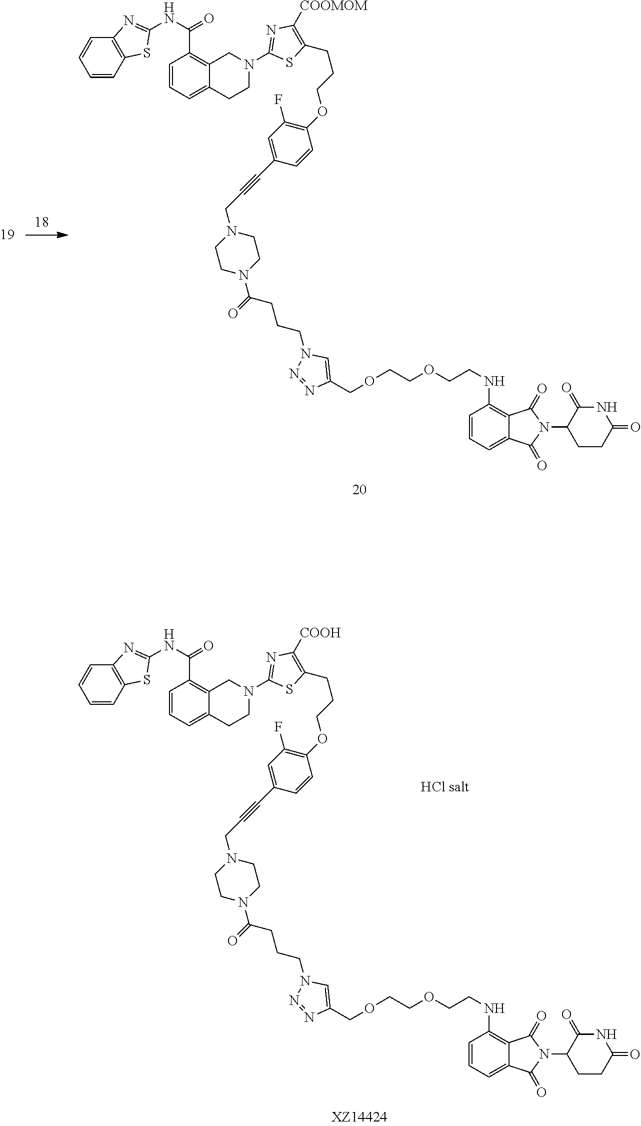

To a mixture of compound 19 (13.0 mg), compound 18 (8.0 mg) in 2 mL t-BuOH-THF (1:1, v/v) under argon was added CuSO₄.5H₂O (0.82 mg) and sodium ascorbate (0.65 mg) in 0.4 mL water. The mixture was stirred at 55° C. for 16 hours and extracted with DCM. The organic phase was washed with brine×1, dried over Na₂SO₄, filtered and evaporated to dryness. The crude product was purified via column chromatography using DCM and methanol as eluents to afford 20 mg compound 20. Yield 91%. Compound 20 (20.0 mg) and 0.1 mL HCl solution (4.0 M in 1,4-dioxane) was stirred in 4 mL DCM-methanol (3:1, v/v) for 3 hours. The solvents were removed under reduced pressure. Et₂O was then added to the residue and the precipitated solid was collected to afford 15.4 mg pure XZ14424. Yield 73%. ¹H NMR (400 MHz, CD₃OD) δ 8.02 (s, 1H), 7.92 (d, J=7.9 Hz, 1H), 7.79 (d, J=7.9 Hz, 2H), 7.61-7.44 (m, 4H), 7.36 (t, J=7.5 Hz, 1H), 7.31-7.18 (m, 2H), 7.12-6.95 (m, 3H), 5.14 (s, 2H), 5.01 (dd, J=12.7, 5.4 Hz, 1H), 4.64 (s, 2H), 4.47 (t, J=6.5 Hz, 2H), 4.34 (s, 2H), 4.16 (t, J=5.5 Hz, 2H), 3.99-3.88 (m, 2H), 3.77-3.44 (m, 14H), 3.38-3.33 (m, 4H), 3.26-3.19 (m, 2H), 2.89-2.62 (m, 3H), 2.52-2.38 (m, 2H), 2.25-2.04 (m, 5H) ppm.

### Example 4: Synthesis of XZ-14418

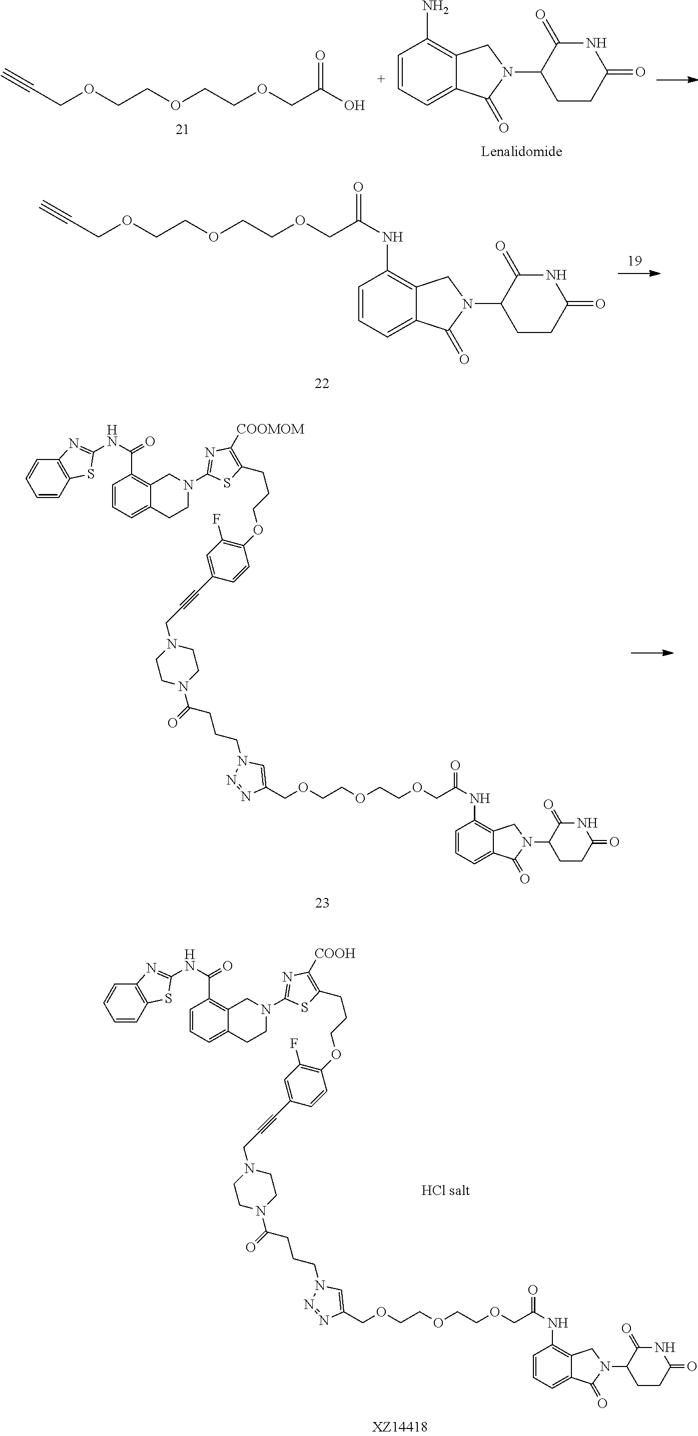

### Preparation of N-(2-(2,6-dioxopiperidin-3-yl)-1-oxoisoindolin-4-yl)-2-(2-(2-(prop-2-ynyloxy)ethoxy)ethoxy)acetamide (22)

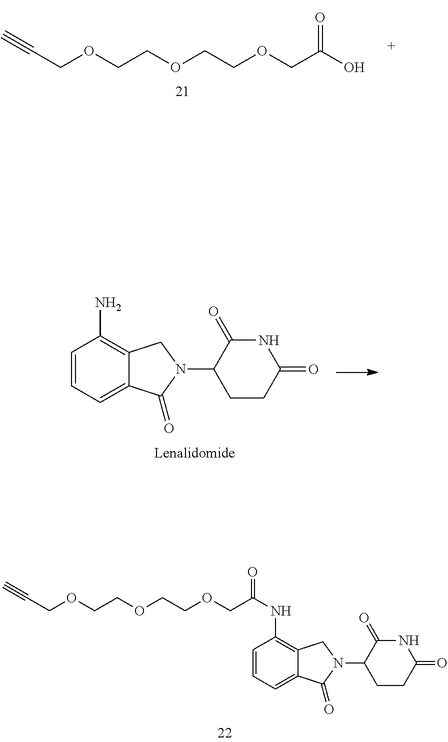

Lenalidomide (61 mg), compound 21 (57 mg), HATU (94 mg) and DIPEA (59 μL) were stirred in 5 mL DCM overnight. The mixture was concentrated under reduced pressure and purified via column chromatography using DCM and methanol as eluents to afford 58 mg compound 22. Yield 56%. ¹H NMR (400 MHz, CDCl₃) δ 8.92 (s, 1H), 7.97 (s, 1H), 7.74 (d, J=7.5 Hz, 1H), 7.68 (d, J=7.9 Hz, 1H), 7.49 (t, J=7.7 Hz, 1H), 5.20 (dd, J=13.3, 5.1 Hz, 1H), 4.45 (s, 2H), 4.14 (d, J=3.4 Hz, 2H), 3.96 (s, 2H), 3.83-3.57 (m, 8H), 2.98-2.70 (m, 2H), 2.49-2.28 (m, 2H), 2.27-2.13 (m, 1H) ppm.

### Preparation of 2-(8-(benzo[d]thiazol-2-ylcarbamoyl)-3,4-dihydroisoquinolin-2(1H)-yl)-5-(3-(4-(3-(4-(4-(4-((2-(2-(2-(2-(2,6-dioxopiperidin-3-yl)-1-oxoisoindolin-4-ylamino)-2-oxoethoxy)ethoxy)ethoxy)methyl)-1H-1,2,3-triazol-1-yl)butanoyl)piperazin-1-yl)prop-1-ynyl)-2-fluorophenoxy)propyl)thiazole-4-carboxylic acid (XZ14418)

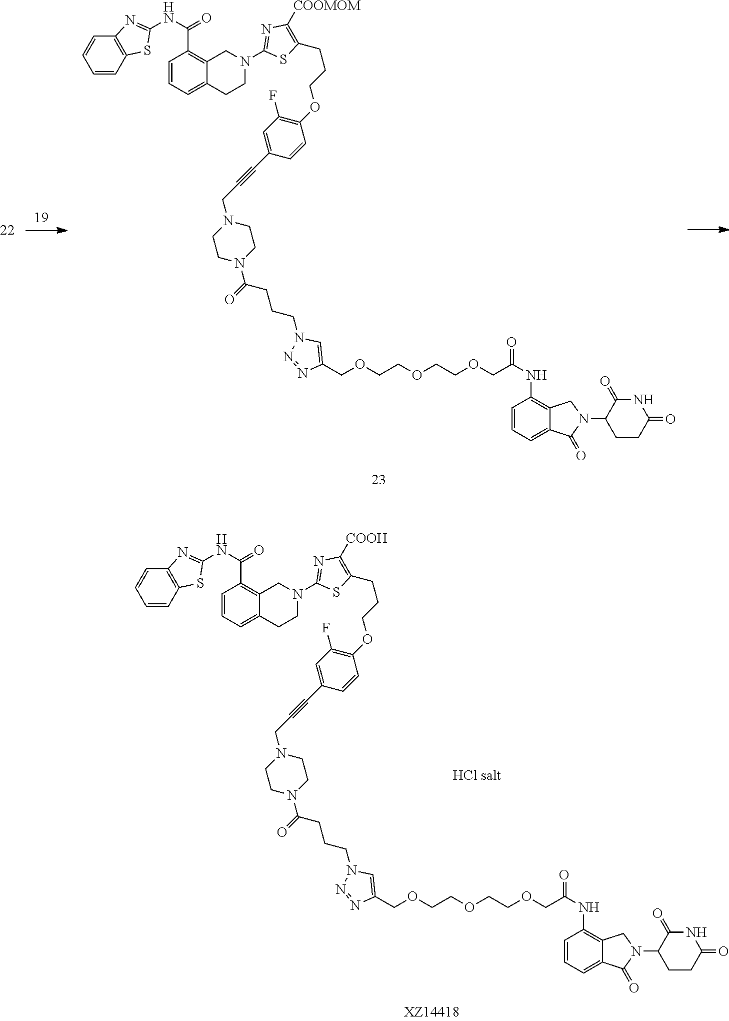

To a mixture of compound 19 (12.0 mg), compound 22 (7.4 mg) in 4 mL t-BuOH-THF (1:3, v/v) under Argon was added CuSO₄.5H₂O (0.70 mg) and sodium ascorbate (0.56 mg) in 0.4 mL water. The mixture was stirred at 55° C. for 16 hours and extracted with DCM. The organic phase was washed with brine×1, dried over Na₂SO₄, filtered and evaporated to dryness. The crude product was purified via column chromatography using DCM and methanol as eluents to afford 15.0 mg compound 23. Yield 85%. Compound 23 (15.0 mg) and 0.1 mL HCl solution (4.0 M in 1,4-dioxane) was stirred in 4 mL 5 mL DCM for 10 minutes. The solvent was removed under reduced pressure. Then Et₂O was added into the residue and the precipitated solid was collected to afford 11.8 mg pure XZ14418. Yield 75%. ¹H NMR (400 MHz, CD₃OD) δ 8.00-7.89 (m, 2H), 7.84-7.76 (m, 2H), 7.69 (d, J=7.8 Hz, 1H), 7.64 (d, J=7.1 Hz, 1H), 7.59-7.43 (m, 4H), 7.37 (t, J=7.6 Hz, 1H), 7.31-7.19 (m, 2H), 7.06 (t, J=8.5 Hz, 1H), 5.23-5.04 (m, 3H), 4.57-4.45 (m, 4H), 4.41 (t, J=6.8 Hz, 2H), 4.33 (s, 2H), 4.23-4.12 (m, 4H), 3.93 (t, J=5.7 Hz, 2H), 3.82-3.63 (m, 10H), 3.37-3.33 (m, 8H), 3.21 (t, J=5.5 Hz, 2H), 2.96-2.67 (m, 2H), 2.57-2.36 (m, 3H), 2.28-2.11 (m, 5H) ppm.

### Example 5: Synthesis of XZ-14455

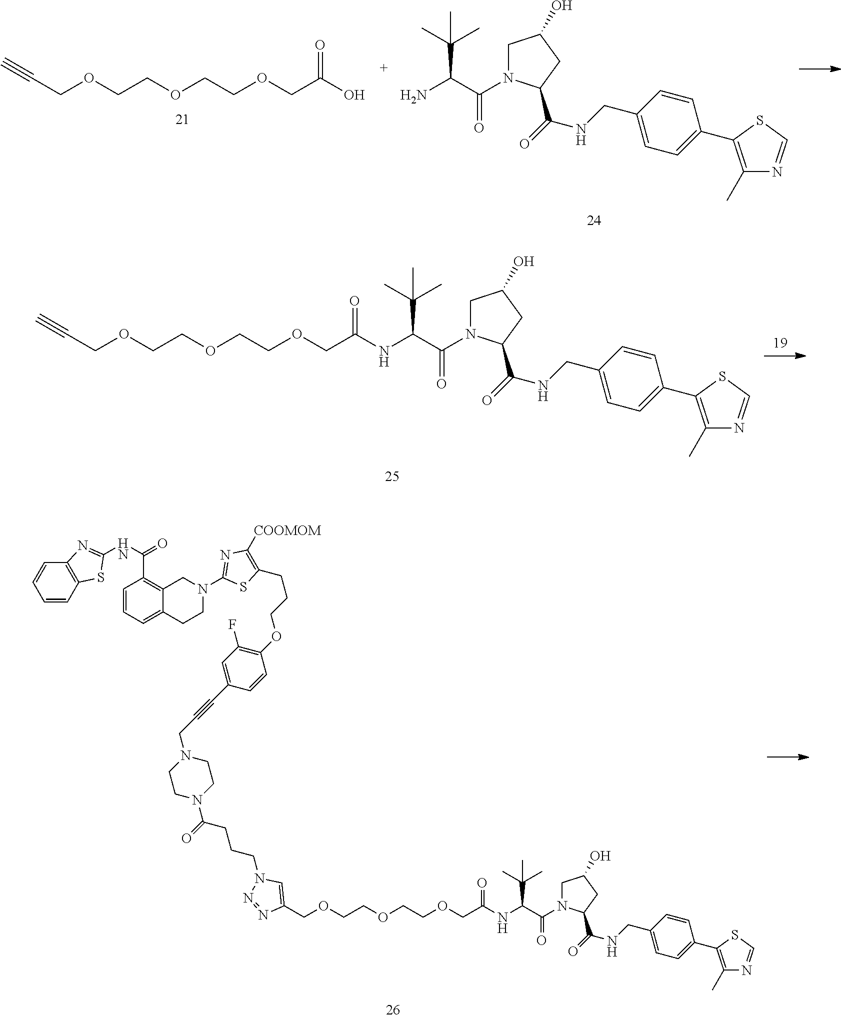

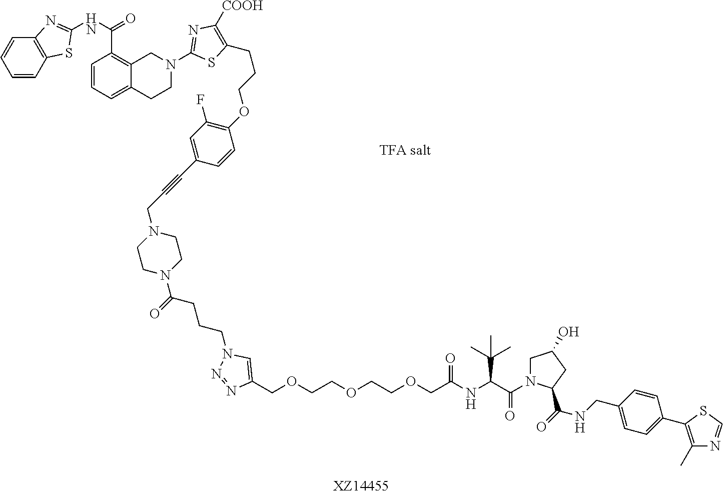 [SMILES: Cc1ncsc1-c1ccc(CNC(=O)[C@@H]2C[C@@H](O)CN2C(=O)[C@@H](NC(=O)COCCOCCOCc2cn(CCCC(=O)N3CCN(CC#Cc4ccc(OCCCc5sc(N6CCc7cccc(C(=O)Nc8nc9ccccc9s8)c7C6)nc5C(=O)O)c(F)c4)CC3)nn2)C(C)(C)C)cc1 ; "TFA salt"]

### Preparation of (2S,4R)-1-((S)-2-tert-butyl-4-oxo-6,9,12-trioxa-3-azapentadec-14-yne)-4-hydroxy-N-(4-(4-methylthiazol-5-yl)benzyl)pyrrolidine-2-carboxamide (25)

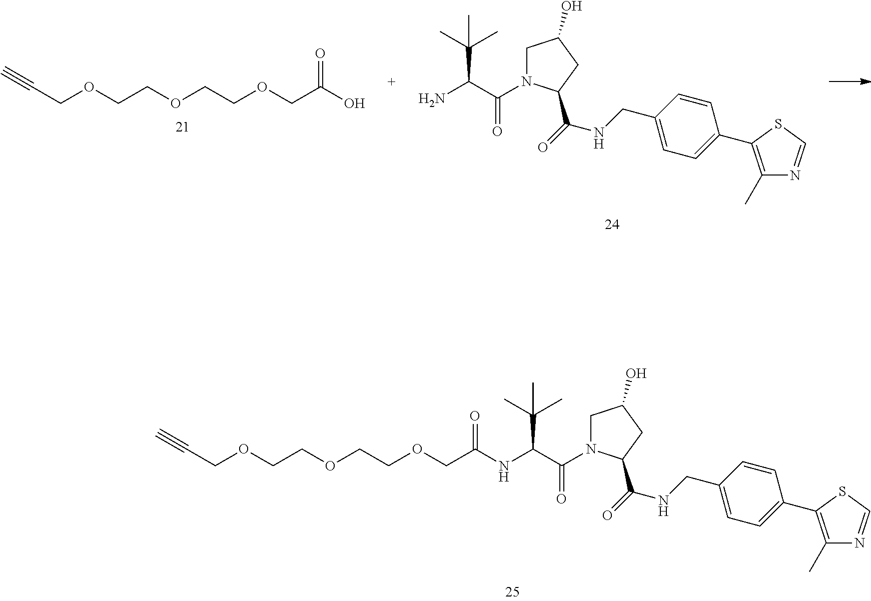

A mixture of compound 21, compound 24, HATU, and DIPEA in DCM was stirred at room temperature overnight. The mixture was concentrated under reduced pressure and purified via column chromatography using DCM and methanol as eluents to afford the title compound. ¹H NMR (400 MHz, CDCl₃) δ 8.68 (s, 1H), 7.60-7.25 (m, 6H), 4.70 (t, J=8.0 Hz, 1H), 4.62-4.37 (m, 3H), 4.33 (dd, J=15.0, 5.3 Hz, 1H), 4.22-4.10 (m, 2H), 4.02-3.91 (m, 3H), 3.65-3.46 (m, 9H), 2.55-2.37 (m, 5H), 2.21-2.09 (m, 1H), 0.91 (s, 9H) ppm.

### Preparation of 2-(8-(benzo[d]thiazol-2-ylcarbamoyl)-3,4-dihydroisoquinolin-2(1H)-yl)-5-(3-(2-fluoro-4-(3-(4-(4-(4-((S)-12-((2S,4R)-4-hydroxy-2-((4-(4-methylthiazol-5-14)benzyl)carbamoyl)pyrrolidine-1-carbonyl)-13,13-dimethyl-10-oxo-2,5,8-trioxa-11-azatetradecyl)-1H-1,2,3-triazol-1-yl)butanoyl)piperazin-1-yl)prop-1-yn-1-yl)phenoxy)propyl)thiazole-4-carboxylic acid (XZ14455)

To a mixture of compound 19 (17.0 mg), compound 25 (17.0 mg) in 4 mL t-BuOH-THF (1:1, v/v) under argon was added CuSO₄.5H₂O (1.0 mg) and sodium ascorbate (0.8 mg) in 0.4 mL water. The mixture was stirred at 50° C. for 5 hours and extracted with DCM. The organic phase was washed with brine×1, dried over Na₂SO₄, filtered and evaporated to dryness. The crude product was purified via column chromatography using DCM and methanol as eluents to afford 14.9 mg compound 26. Yield 51%. Compound 26 (3.5 mg) and 0.1 mL TFA was stirred in 2 mL DCM for 6 hours. The solvent was removed under reduced pressure. Then Et₂O was added into the residue. The precipitated solid was filtered and washed with EtOAc followed by DCM-hexanes (1:1 v/v) to afford 3.5 mg pure XZ14455. Yield 83%. ¹H NMR (400 MHz, CD₃OD) δ 8.86 (s, 1H), 8.00-7.89 (m, 2H), 7.78 (d, J=7.9 Hz, 1H), 7.71-7.59 (m, 1H), 7.53-7.30 (m, 8H), 7.29-7.17 (m, 2H), 7.02 (t, J=8.5 Hz, 1H), 4.89 (s, 2H), 4.73-4.20 (m, 12H), 4.17-3.96 (m, 4H), 3.92-3.59 (m, 15H), 3.40-3.33 (m, 2H), 3.28-3.23 (m, 2H), 3.07 (t, J=5.8 Hz, 2H), 2.49-2.36 (m, 5H), 2.31-1.94 (m, 6H), 1.02 (s, 9H) ppm.

### Example 6: Synthesis of XZ-14439

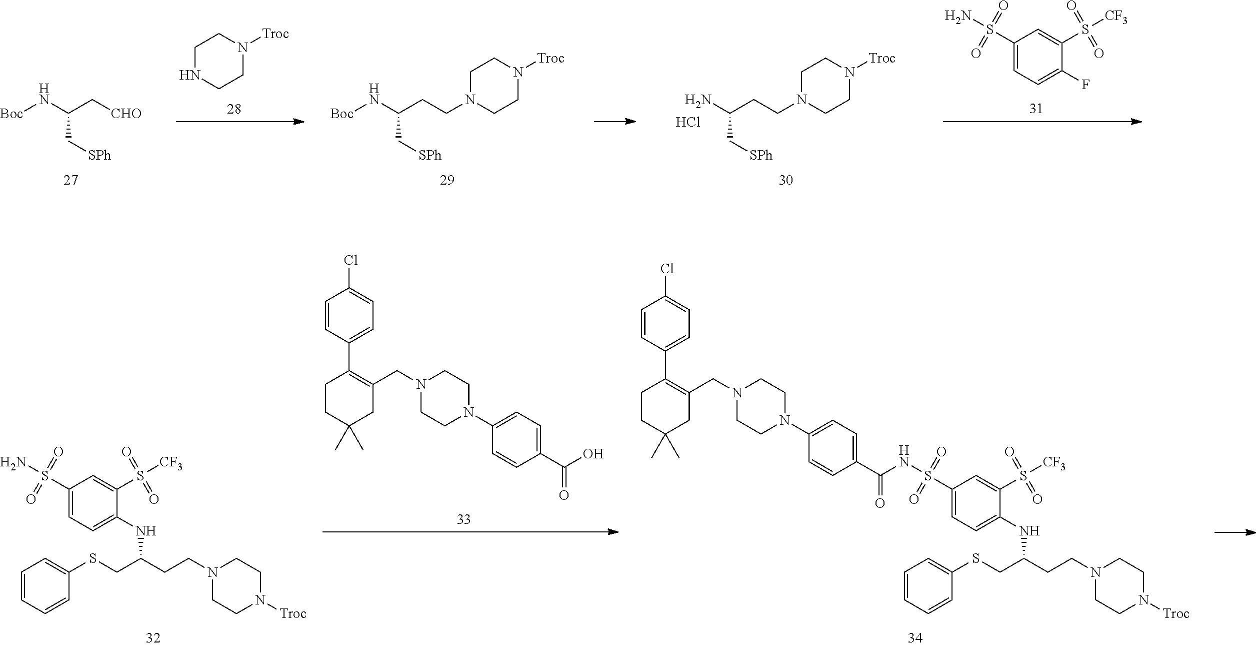

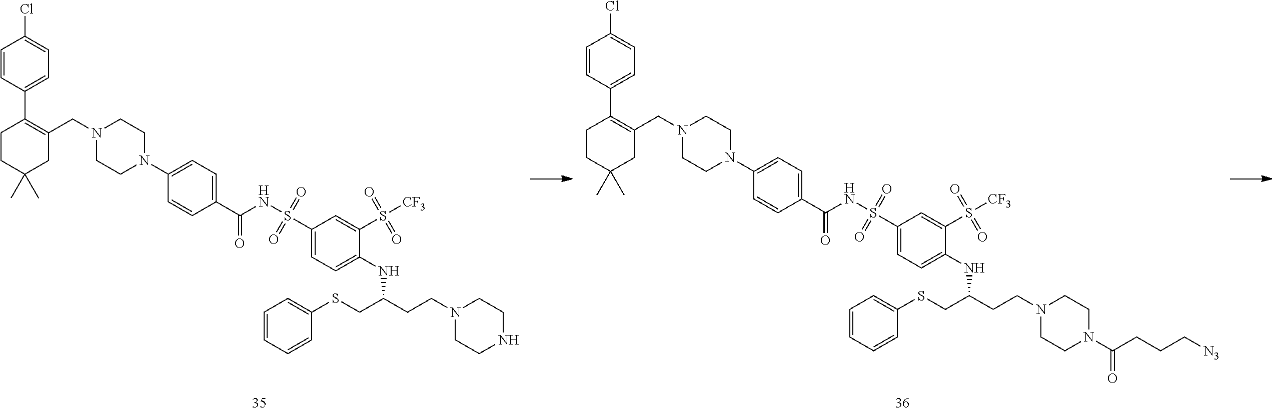 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)CCCN=[N+]=[N-])CC5)CSc5ccccc5)c(S(=O)(=O)C(F)(F)F)c4)cc3)CC2)C1]

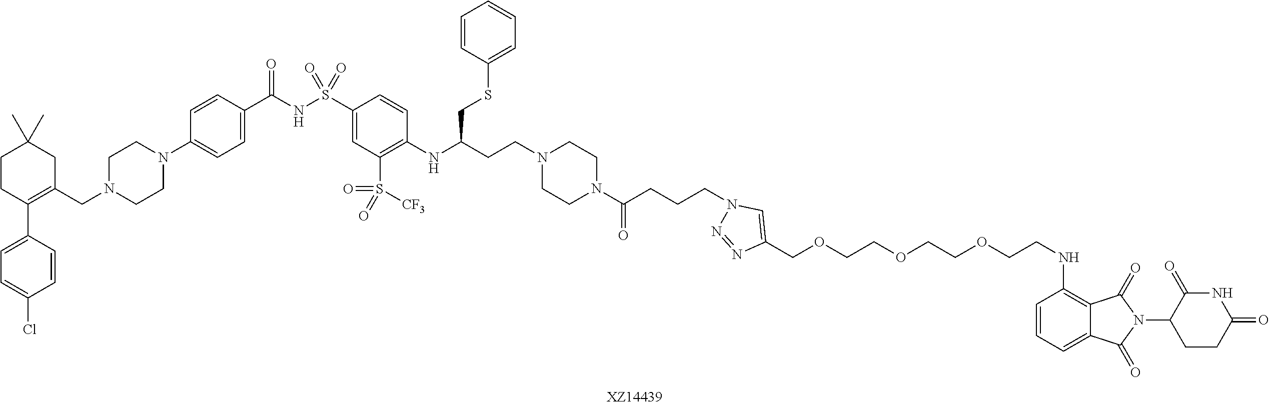 [SMILES: CC1(C)CCC(c2ccc(Cl)cc2)=C(CN2CCN(c3ccc(C(=O)NS(=O)(=O)c4ccc(N[C@H](CCN5CCN(C(=O)CCCn6cc(COCCOCCOCCNc7cccc8c7C(=O)N(C7CCC(=O)NC7=O)C8=O)nn6)CC5)CSc5ccccc5)c(S(=O)(=O)C(F)(F)F)c4)cc3)CC2)C1]

### Preparation of 2,2,2-trichloroethyl (R)-4-(3-((tert-butoxycarbonyl)amino)-4-(phenylthio)butyl)piperazine-1-carboxylate (29)

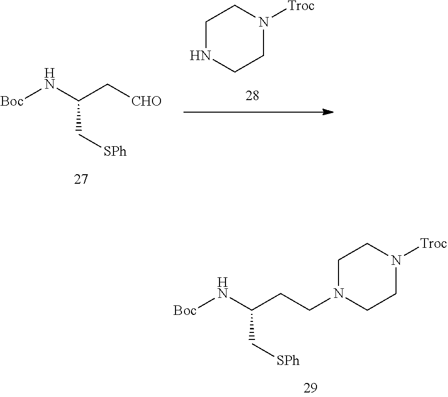

To a mixture of compound 27 (592 mg), compound 28 (753 mg), and TEA (1.12 mL) in 15 mL DCM was added 638 mg NaBH(OAc)₃. The solution was stirred at room temperature for 1 hour. Then it was poured into water and extracted with DCM. The organic phase was washed with brine×1, dried over Na₂SO₄, filtered and evaporated to dryness. The crude product was purified via column chromatography using EtOAc and hexanes as eluents to afford 733 mg compound 29. Yield 68%. ¹H NMR (400 MHz, CDCl₃) δ 7.43-7.36 (m, 2H), 7.32-7.27 (m, 2H), 7.19 (t, J=7.3 Hz, 1H), 5.44 (br s, 1H), 4.76 (s, 2H), 3.99-3.84 (m, 1H), 3.72-3.49 (m, 4H), 3.23 (dd, J=13.3, 4.6 Hz, 1H), 3.10-2.95 (m, 1H), 2.61-2.31 (m, 6H), 1.96-1.61 (m, 2H), 1.43 (s, 9H) ppm.

### Preparation of 2,2,2-trichloroethyl (R)-4-(3-amino-4-(phenylthio)butyl)piperazine-1-carboxylate (30)

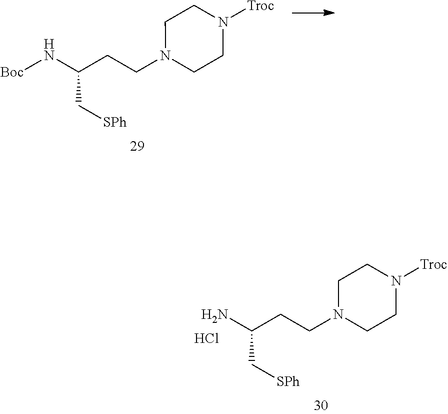

To a mixture of compound 29 (733 mg) in 5 mL DCM was added 5 mL HCl solution (4.0 M in 1,4-dioxane). The mixture was stirred at room temperature for 1 hour and the solvents were removed under reduced pressure. The solid was washed with Et₂O to afford 647 mg compound 30 as white solid. Yield 99%. ¹H NMR (400 MHz, CDCl₃) δ 7.41-7.33 (m, 2H), 7.31-7.26 (m, 2H), 7.23-7.15 (m, 1H), 4.74 (s, 2H), 3.73-3.41 (m, 4H), 3.20-2.66 (m, 5H), 2.58-2.28 (m, 6H), 1.84-1.57 (m, 2H) ppm. Preparation of 2,2,2-trichloroethyl (R)-4-(4-(phenylthio)-3-((4-sulfamoyl-2-((trifluoromethyl)sulfonyl)phenyl)amino)butyl)piperazine-1-carboxylate (32)

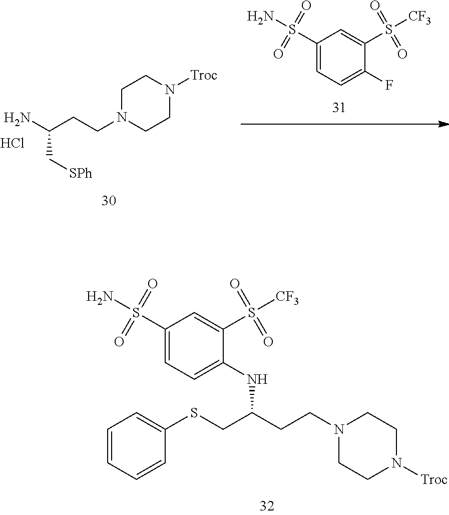

A mixture of compound 30 (647 mg), 4-fluoro-3-((trifluoromethyl)sulfonyl)benzenesulfonamide 31 (417 mg) and TEA (945 μL) in 20 mL acetonitrile was refluxed for 4 hours. The solvent was evaporated under reduced pressure and the crude product was purified via column chromatography using EtOAc and hexanes as eluents to afford 780 mg compound 32 as white solid. Yield 79%. ¹H NMR (400 MHz, CDCl₃) δ 8.24 (d, J=2.2 Hz, 1H), 7.84 (d, J=9.1 Hz, 1H), 7.42-7.37 (m, 2H), 7.36-7.27 (m, 3H), 7.05 (d, J=8.6 Hz, 1H), 6.65 (br s, 1H), 5.13 (br s, J=10.8 Hz, 2H), 4.76 (s, 2H), 3.94 (s, 1H), 3.58 (s, 4H), 3.16-2.97 (m, 2H), 2.82-2.26 (m, 6H), 2.17 (s, 1H), 1.77 (s, 1H) ppm.

### Preparation of 2,2,2-trichloroethyl (R)-4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carboxylate (34)

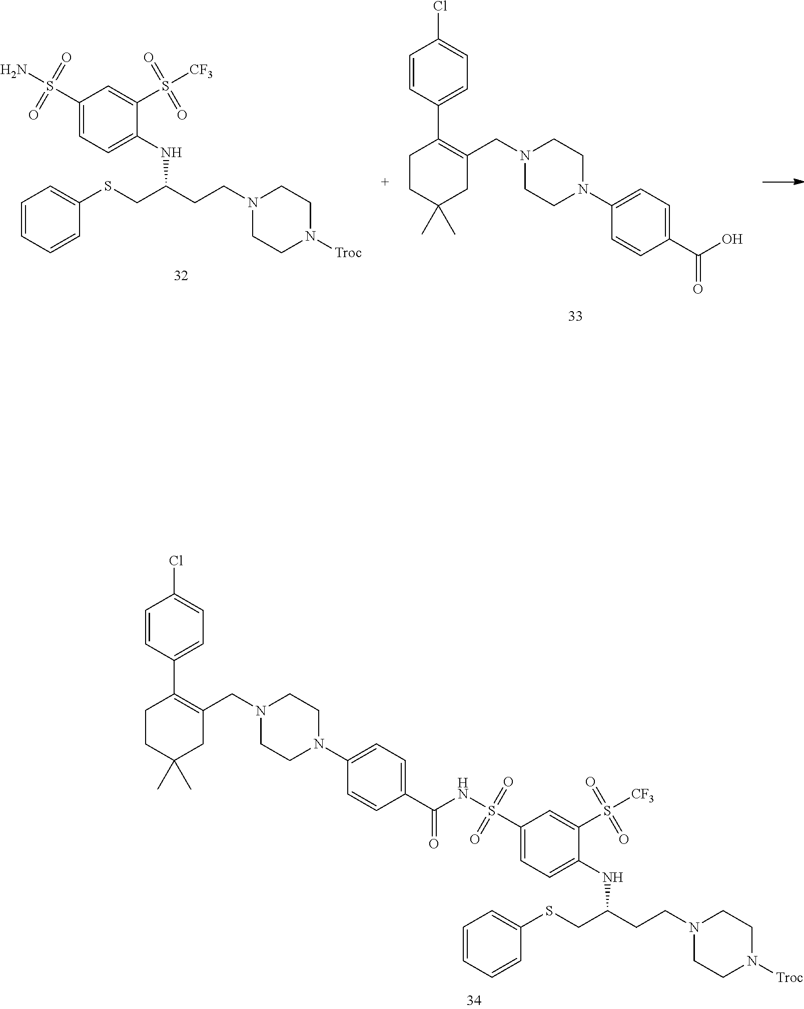

A mixture of compound 32 (780 mg), 4-(4-((4′-chloro-4,4-dimethyl-3,4,5,6-tetrahydro-[1,1′-biphenyl]-2-yl)methyl)piperazin-1-yl)benzoic acid 33 (470 mg), EDCl (411 mg) and DMAP (262 mg) in DCM was stirred at room temperature overnight. The solvent was evaporated under reduced pressure and the crude product was purified via column chromatography using DCM and methanol as eluents to afford 859 mg compound 34 as white solid. Yield 70%. ¹H NMR (400 MHz, CDCl₃) δ 8.35 (s, 1H), 8.10 (d, J=8.7 Hz, 1H), 7.66 (d, J=8.0 Hz, 2H), 7.43-7.18 (m, 7H), 7.12-6.96 (m, 3H), 6.74 (s, 1H), 6.56 (d, J=7.9 Hz, 1H), 4.74 (s, 2H), 3.93-3.83 (m, 1H), 3.61-3.42 (m, 4H), 3.39-3.25 (m, 4H), 3.16-2.83 (m, 4H), 2.44-2.02 (m, 15H), 1.77-1.60 (m, 1H), 1.56-1.42 (m, 2H), 0.98 (s, 6H) ppm.

### Preparation of (R)-4-(4-((4′-chloro-4,4-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (35)

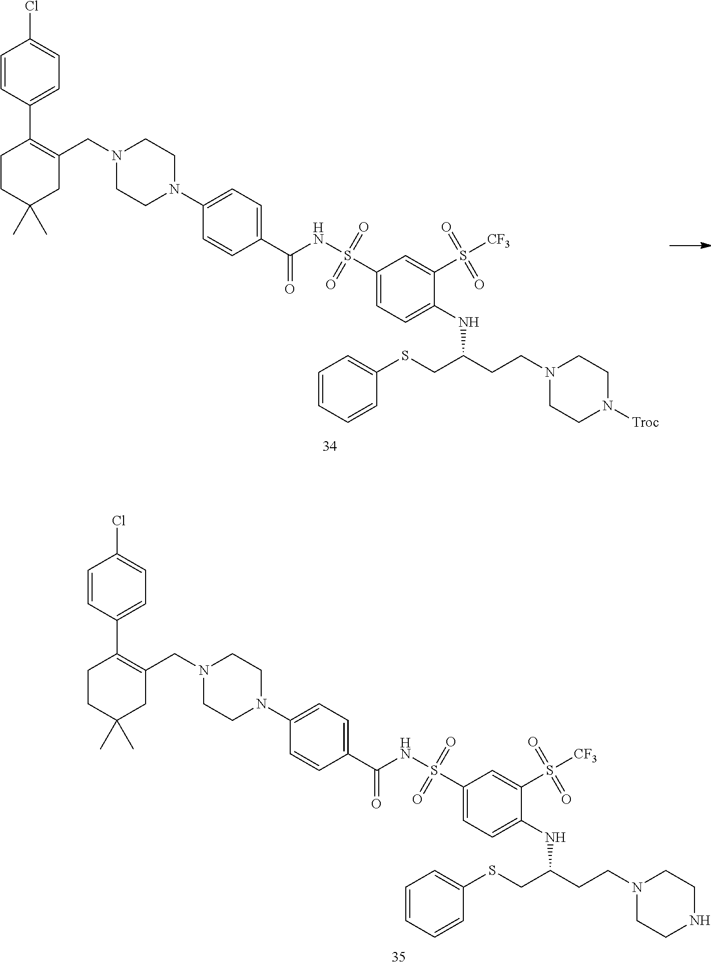

Zinc powder (960 mg) was added to a mixture of compound 34 (316 mg) and AcOH (600 μL) in 20 mL THF. The reaction was stirred at room temperature for 5 hours. The solid was removed by filtration and the filtrate was poured into water and extracted with EtOAc. The organic phase was washed with brine×1, dried over Na₂SO₄, filtered and evaporated to dryness. The crude product was purified via column chromatography using DCM, methanol, and TEA as eluents to afford 210 mg compound 35. Yield 78%. ¹H NMR (400 MHz, CDCl₃) δ 8.21 (s, 1H), 7.93 (d, J=9.2 Hz, 1H), 7.85 (d, J=8.6 Hz, 2H), 7.33-7.24 (m, 2H), 7.22-7.15 (m, 6H), 7.15-7.08 (m, 1H), 6.92 (d, J=8.3 Hz, 2H), 6.77 (d, J=8.4 Hz, 1H), 6.66 (d, J=8.7 Hz, 2H), 6.46 (d, J=9.3 Hz, 1H), 3.83-3.67 (m, 1H), 3.17-3.08 (m, 4H), 3.02-2.92 (m, 5H), 2.89-2.78 (m, 1H), 2.72 (s, 2H), 2.64-2.13 (m, 12H), 2.04-1.91 (m, 3H), 1.62-1.49 (m, 1H), 1.39 (t, J=6.3 Hz, 2H), 0.91 (s, 6H) ppm.

### Preparation of (R)—N-((4-((4-(4-(4-azidobutanoyl)piperazin-1-yl)-1-(phenylthio)butan-2-14)amino)-3-((trifluoromethyl)sulfonyl)phenyl)sulfonyl)-4-(4-((4′-chloro-4,4-dimethyl-3,4,5,6-tetrahydro-[1,1′-biphenyl]-2-yl)methyl)piperazin-1-yl)benzamide (36)

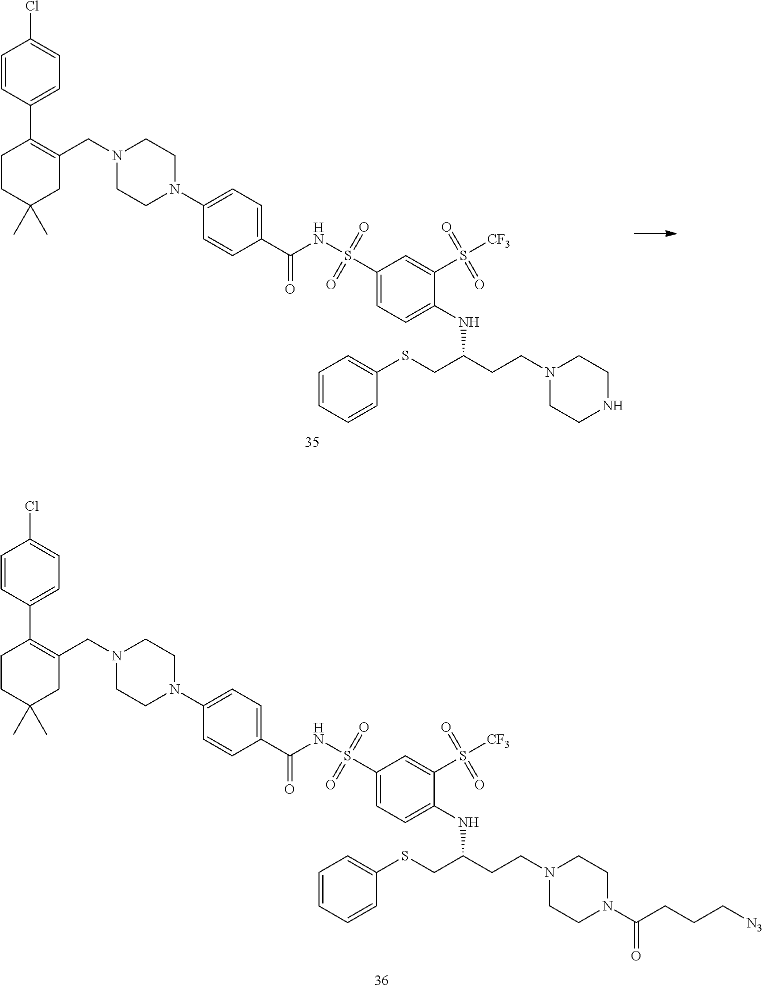 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)CCCN=[N+]=[N-])CC5)CSc5ccccc5)c(S(=O)(=O)C(F)(F)F)c4)cc3)CC2)C1]

HATU (30 mg) was added to a mixture of compound 35 (50 mg), 4-azidobutanoic acid (6.7 mg), DIPEA (13.5 μL) in 2 mL DCM. The mixture was stirred at room temperature for 1 hour. The solvent was removed under reduced pressure and the crude product was purified via column chromatography using DCM and methanol as eluents to afford 40 mg compound 36. Yield 72%. ¹H NMR (400 MHz, CDCl₃) δ 8.35 (d, J=2.2 Hz, 1H), 8.11 (dd, J=9.2, 2.2 Hz, 1H), 7.67 (d, J=8.9 Hz, 2H), 7.40-7.35 (m, 2H), 7.34-7.27 (m, 3H), 7.26-7.24 (m, 2H), 7.09 (d, J=8.5 Hz, 1H), 7.02-6.96 (m, 2H), 6.76 (d, J=9.0 Hz, 2H), 6.58 (d, J=9.4 Hz, 1H), 3.99-3.81 (m, 1H), 3.72-3.60 (m, 1H), 3.53-3.33 (m, 5H), 3.32-3.22 (m, 4H), 3.11 (dd, J=13.8, 4.9 Hz, 1H), 3.00 (dd, J=13.8, 7.5 Hz, 1H), 2.87 (s, 2H), 2.51-2.20 (m, 14H), 2.19-2.08 (m, 1H), 2.06-1.99 (m, 2H), 1.97-1.85 (m, 2H), 1.71-1.64 (m, 1H), 1.46 (t, J=6.4 Hz, 2H), 0.97 (s, 6H) ppm.

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(4-(4-((2-(2-(2-((2-(2,6-dioxopiperidin-3-yl)-1,3-dioxoisoindolin-4-yl)amino)ethoxy)ethoxy)ethoxy)methyl)-1H-1,2,3-triazol-1-yl)butanoyl)piperazin-1-yl)-1-(phenylthio)butan-2-yl)amino)-3-((trifluoromethyl)sulfonyl)phenyl)sulfonyl)benzamide (XZ-14439)

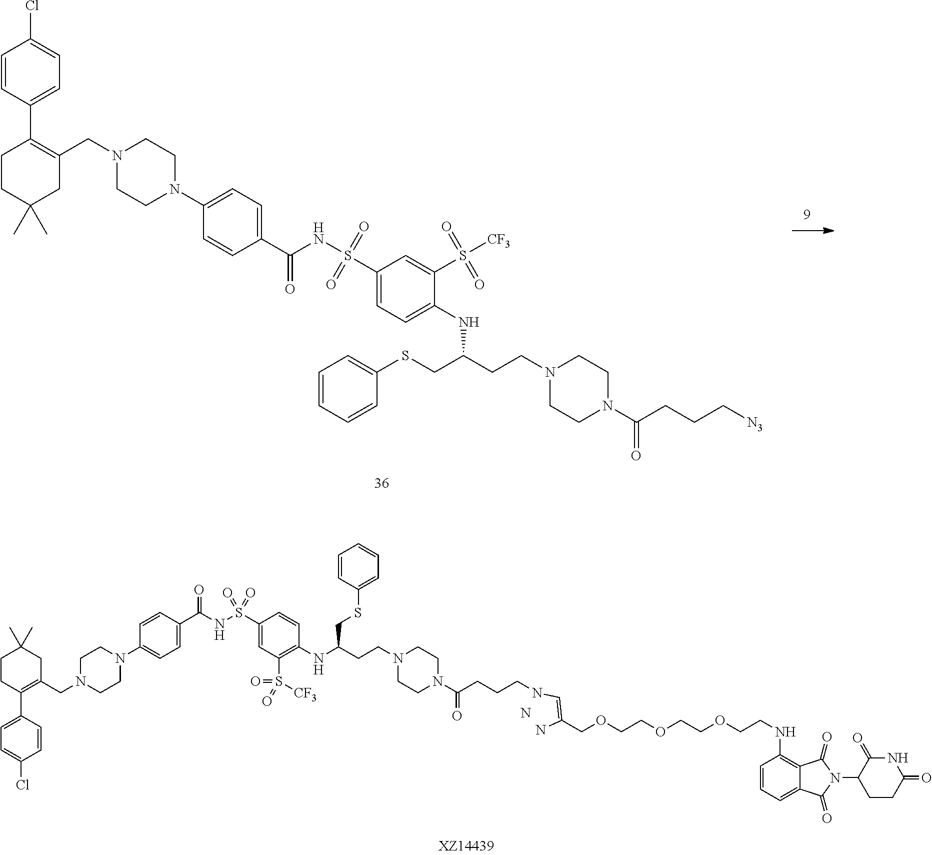

To a mixture of compound 36 (7.5 mg), compound 9 (3.7 mg) in 2 mL t-BuOH-THF (1:1, v/v) under argon was added CuSO₄.5H₂O (0.35 mg) and sodium ascorbate (0.28 mg) in 0.4 mL water. The mixture was stirred at 50° C. for 3 hours and extracted with DCM. The organic phase was washed with brine×1, dried over Na₂SO₄, filtered and evaporated to dryness. The crude product was purified via column chromatography using DCM and methanol as eluents to afford 5.9 mg XZ14439. Yield 56%. ¹H NMR (400 MHz, CDCl₃ and CD₃OD) δ 8.33 (s, 1H), 8.10 (d, J=7.8 Hz, 1H), 7.79-7.65 (m, 3H), 7.54-7.44 (m, 1H), 7.43-7.35 (m, 2H), 7.33-7.32 (m, 1H), 7.30-7.22 (m, 4H), 7.12-7.02 (m, 2H), 6.99 (d, J=8.3 Hz, 2H), 6.92 (d, J=8.6 Hz, 1H), 6.77 (d, J=8.9 Hz, 2H), 6.61 (d, J=9.3 Hz, 1H), 5.02-4.85 (m, 1H), 4.66 (s, 2H), 4.41 (t, J=6.6 Hz, 2H), 4.00-3.79 (m, 1H), 3.80-3.58 (m, 12H), 3.52-3.38 (m, 4H), 3.31-3.23 (m, 4H), 3.12 (dd, J=13.8, 5.0 Hz, 1H), 3.02 (dd, J=13.9, 7.3 Hz, 1H), 2.84-2.77 (m, 5H), 2.50-2.06 (m, 18H), 2.01 (s, 2H), 1.74-1.63 (m, 1H), 1.46 (t, J=6.4 Hz, 2H), 0.98 (s, 6H) ppm.

### Example 7: Synthesis of PZ-15227

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(4-(4-((2-(2-((2-(2,6-dioxopiperidin-3-yl)-1,3-dioxoisoindolin-4-yl)amino)ethoxy)ethoxy)methyl)-1H-1,2,3-triazol-1-yl)butanoyl)piperazin-1-yl)-1-(phenylthio)butan-2-yl)amino)-3-((trifluoromethyl)sulfonyl)phenyl)sulfonyl)benzamide (PZ-15227)

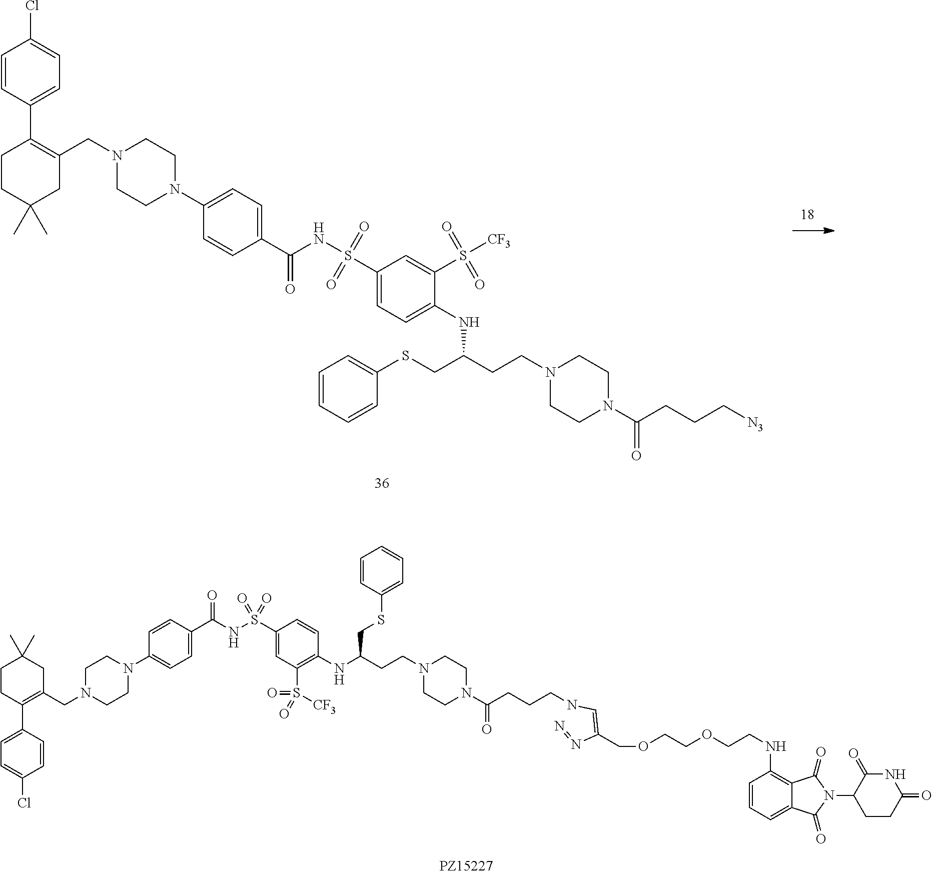 [SMILES: CC1(C)CCC(c2ccc(Cl)cc2)=C(CN2CCN(c3ccc(C(=O)NS(=O)(=O)c4ccc(N[C@H](CCN5CCN(C(=O)CCCN=[N+]=[N-])CC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)CCCN/C=C(/COCCOCCNc6cccc7c6C(=O)N(C6CCC(=O)NC6=O)C7=O)N=N)CC5)CSc5ccccc5)c(S(=O)(=O)C(F)(F)F)c4)cc3)CC2)C1]

To a mixture of compound 36 (25.0 mg), compound 18 (11.0 mg) in 3 mL t-BuOH-THF (1:2, v/v) under Argon was added CuSO₄.5H₂O (1.15 mg) and sodium ascorbate (0.91 mg) in 0.3 mL water. The mixture was stirred at 50° C. overnight and extracted with DCM. The organic phase was washed with brine×1, dried over Na₂SO₄, filtered and evaporated to dryness. The crude product was purified via column chromatography using DCM and methanol as eluents to afford 23 mg PZ15227. Yield 67%. ¹H NMR (400 MHz, CDCl₃ and CD₃OD) δ 9.05 (brs, 1H), 8.36 (s, 1H), 8.10 (d, J=7.8 Hz, 1H), 7.79-7.64 (m, 3H), 7.54-7.42 (m, 1H), 7.43-7.22 (m, 7H), 7.10-7.02 (m, 2H), 6.99 (d, J=7.2 Hz, 2H), 6.92 (d, J=8.8 Hz, 1H), 6.77 (d, J=8.8 Hz, 2H), 6.61 (d, J=9.3 Hz, 1H), 6.50 (brs, 1H), 4.99-4.85 (m, 1H), 4.69 (s, 2H), 4.42-4.37 (m, 2H), 4.00-3.77 (m, 1H), 3.80-3.58 (m, 8H), 3.52-3.20 (m, 8H), 3.12-3.00 (m, 2H), 2.84-2.75 (m, 5H), 2.45-1.98 (m, 20H), 1.74-1.60 (m, 1H), 1.46 (t, J=6.4 Hz, 2H), 0.97 (s, 6H) ppm.

### Example 8: Synthesis of XZ-14509

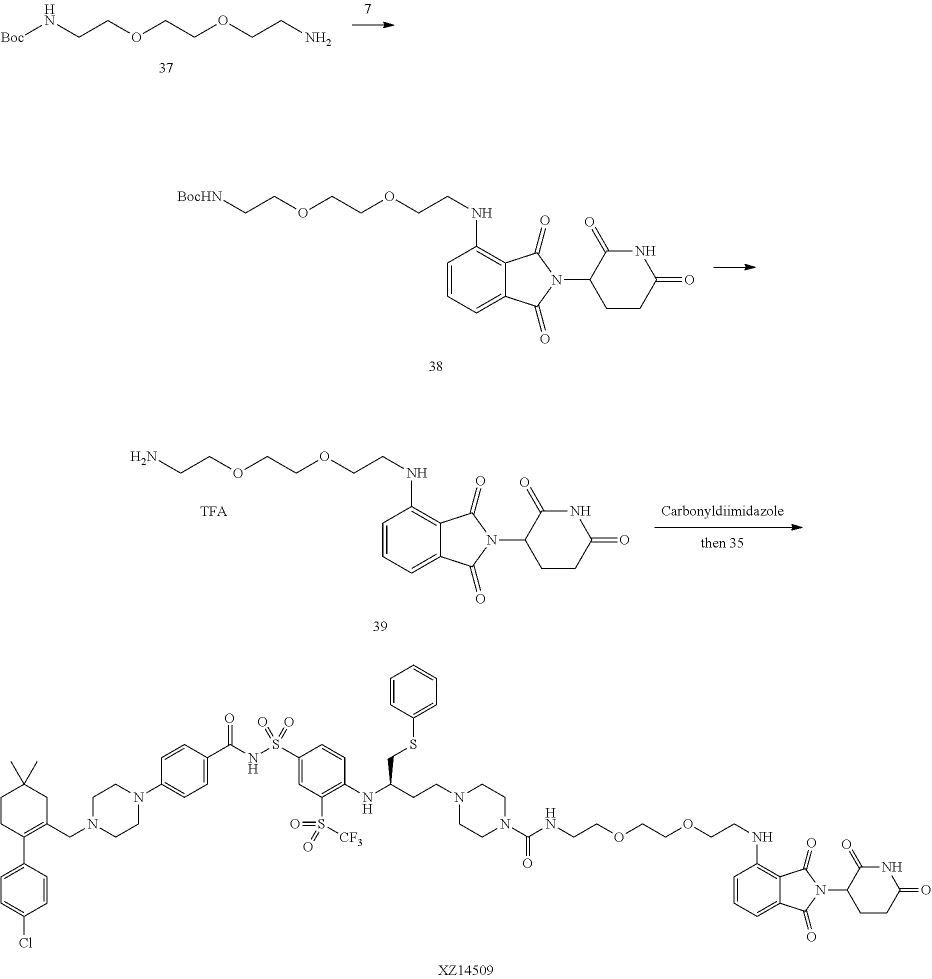 [SMILES: CC(C)(C)OC(=O)NCCOCCOCCN ; CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O ; CC1(C)CCC(c2ccc(Cl)cc2)=C(CN2CCN(c3ccc(C(=O)NS(=O)(=O)c4ccc(N[C@H](CCN5CCN(C(=O)NCCOCCOCCNc6cccc7c6C(=O)N(C6CCC(=O)NC6=O)C7=O)CC5)CSc5ccccc5)c(S(=O)(=O)C(F)(F)F)c4)cc3)CC2)C1]

### Preparation of 4-((2-(2-(2-aminoethoxy)ethoxy)ethyl)amino)-2-(2-oxopiperidin-3-yl)isoindoline-1,3-dione (39)

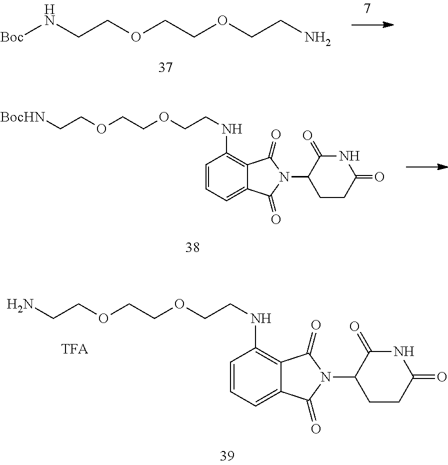 [SMILES: CC(C)(C)OC(=O)NCCOCCOCCN ; CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O]

Compound 7 (200 mg), amine 37 (178 mg), and DIPEA (240 μL) in 5 mL DMF were stirred at 90° C. for 16 hours. Water was added to the reaction mixture and extracted with EtOAc. The organic phase was washed with water×1, brine×1, dried over Na₂SO₄, filtered and evaporated to dryness. The resulting mixture was purified by column chromatography to afford 183 mg compound 38. Yield 50%. To a mixture of compound 38 (160 mg) in 5 mL DCM was added 0.5 mL TFA. The mixture was stirred at room temperature for 2 h and the solvent was evaporated under reduced pressure. The salt was washed with Et₂O to afford pure compound 39. ¹H NMR (400 MHz, CDCl₃) δ 9.63 (br s, 1H), 7.82 (br s, 2H), 7.48 (dd, J=8.4, 7.2 Hz, 1H), 7.04 (d, J=7.0 Hz, 1H), 6.87 (d, J=8.5 Hz, 1H), 5.13-4.82 (m, 1H), 3.85-3.60 (m, 8H), 3.55-3.37 (m, 2H), 3.28-3.12 (m, 2H), 2.81-2.58 (m, 3H), 2.09-1.88 (m, 1H) ppm.

### Preparation of 4-((R)-3-(4-(N-(4-(4-((2-(4-chlorophenyl)-5,5-dimethylcyclohex-1-enyl)methyl)piperazin-1-yl)benzoyl)sulfamoyl)-2-(trifluoromethylsulfonyl)phenylamino)-4-(phenylthio)butyl)-N-(2-(2-(2-(2-(2,6-dioxopiperidin-3-yl)-1,3-dioxoisoindolin-4-ylamino)ethoxy)ethoxy)ethyl)piperazine-1-carboxamide (XZ-14509)

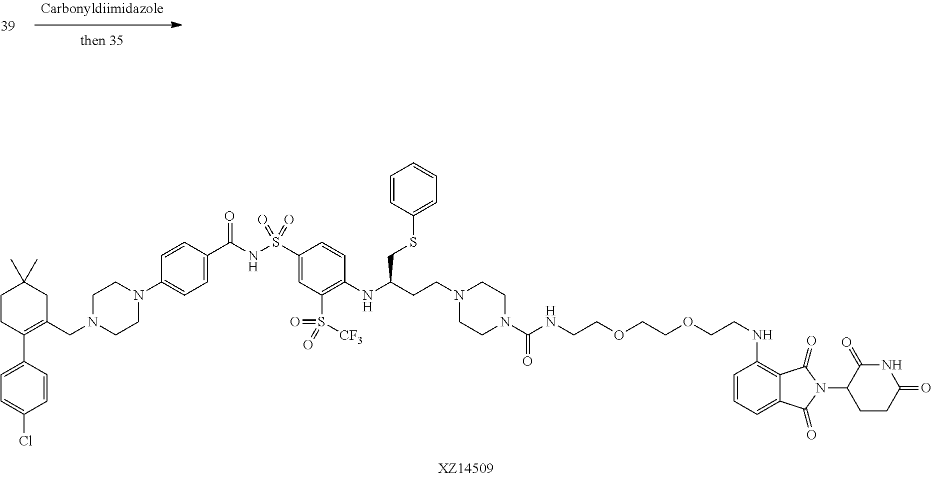 [SMILES: CC1(C)CCC(c2ccc(Cl)cc2)=C(CN2CCN(c3ccc(C(=O)NS(=O)(=O)c4ccc(N[C@H](CCN5CCN(C(=O)NCCOCCOCCNc6cccc7c6C(=O)N(C6CCC(=O)NC6=O)C7=O)CC5)CSc5ccccc5)c(S(=O)(=O)C(F)(F)F)c4)cc3)CC2)C1]

A mixture of compound 39 (20 mg), carbonyldiimidazole (CDI) (10 mg) and TEA (7.0 μL) in 3 mL DCM was stirred at room temperature for 2 hours. Compound 35 (15 mg) and DIPEA (0.05 mL) were then added into the above solution. The mixture was stirred overnight and quenched by the addition of NH₄Cl (aq.), extracted with DCM and the organic phase was washed with water×1, brine×1, dried over Na₂SO₄, filtered and evaporated to dryness. The crude product was purified by column chromatography using DCM and methanol as eluents to afford 6.7 mg compound XZ14509. Yield 31%. ¹H NMR (400 MHz, CDCl₃) δ 9.24 (brs, 1H), 8.35 (s, 1H), 8.14-7.98 (m, 1H), 7.81-7.62 (m, 2H), 7.52-7.40 (m, 1H), 7.39-7.27 (m, 4H), 7.24-7.15 (m, 1H), 7.12-6.94 (m, 4H), 6.87 (d, J=8.6 Hz, 1H), 6.73 (d, J=7.2 Hz, 2H), 6.66-6.57 (m, 1H), 6.56-6.46 (m, 1H), 5.20-5.02 (brs, 1H), 5.00-4.83 (m, 1H), 3.95-3.81 (m, 1H), 3.75-3.69 (m, 2H), 3.67-3.61 (m, 4H), 3.61-3.53 (m, 2H), 3.49-3.38 (m, 4H), 3.38-3.18 (m, 8H), 3.12-2.95 (m, 2H), 2.88-2.66 (m, 5H), 2.47-2.18 (m, 12H), 2.17-1.98 (m, 4H), 1.69-1.57 (m, 1H), 1.46 (t, J=6.3 Hz, 2H), 0.97 (s, 6H) ppm.

### Example 9: Synthesis of XZ-14516

### Preparation of 4-(4-((2-(4-chlorophenyl)-5,5-dimethylcyclohex-1-enyl)methyl)piperazin-1-yl)-N-(4-((2R)-4-(4-(2-(2-(2-(2-(2,6-dioxopiperidin-3-yl)-1,3-dioxoisoindolin-4-ylamino)ethoxy)ethoxy)ethylcarbamothioyl)piperazin-1-yl)-1-(phenylthio)butan-2-ylamino)-3-(trifluoromethylsulfonyl)phenylsulfonyl)benzamide (XZ-14516)

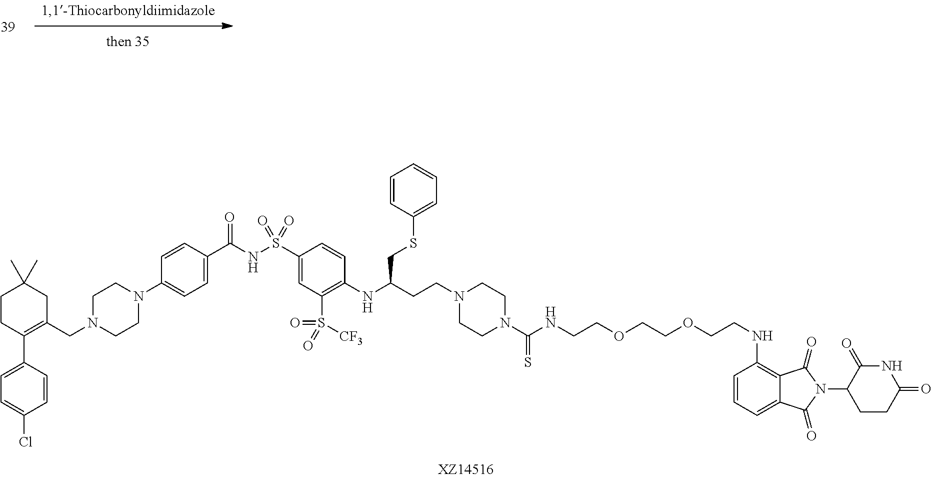 [SMILES: CC1(C)CCC(c2ccc(Cl)cc2)=C(CN2CCN(c3ccc(C(=O)NS(=O)(=O)c4ccc(N[C@H](CCN5CCN(C(=S)NCCOCCOCCNc6cccc7c6C(=O)N(C6CCC(=O)NC6=O)C7=O)CC5)CSc5ccccc5)c(S(=O)(=O)C(F)(F)F)c4)cc3)CC2)C1]

A mixture of compound 39 (12 mg), 1,1′-thiocarbonyldiimidazole (6 mg) and TEA (4.2 μL) in 2 mL DCM was stirred at room temperature for 1 hour. Then compound 35 (6.5 mg) and DIPEA (0.05 mL) were added into the above solution. The mixture was stirred overnight and quenched by the addition of NH₄Cl (aq). Subsequently, it was with DCM and the organic phase was washed with water×1, brine×1, dried over Na₂SO₄, filtered and evaporated to dryness. The crude product was purified by column chromatography using DCM and methanol as eluents to afford 6.4 mg compound XZ14516. Yield 68%. ¹H NMR (400 MHz, CDCl₃) δ 8.90 (brs, 1H), 8.37 (d, J=2.1 Hz, 1H), 8.15-8.00 (m, 1H), 7.65 (d, J=7.6 Hz, 2H), 7.51-7.42 (m, 1H), 7.38-7.27 (m, 5H), 7.12-7.05 (m, 2H), 6.99 (d, J=8.4 Hz, 2H), 6.87 (d, J=8.5 Hz, 1H), 6.75 (dd, J=9.0, 3.8 Hz, 2H), 6.62 (dd, J=12.9, 9.5 Hz, 1H), 6.51 (t, J=5.2 Hz, 1H), 6.22 (brs, 1H), 4.95-4.80 (m, 1H), 3.90-3.61 (m, 15H), 3.48-3.41 (m, 2H), 3.30-3.20 (m, 4H), 3.13-2.96 (m, 2H), 2.88-2.71 (m, 5H), 2.47-2.22 (m, 12H), 2.13-2.00 (m, 4H), 1.75-1.70 (m, 1H), 1.46 (t, J=6.4 Hz, 2H), 0.99 (s, 6H) ppm.

### Example 10: Synthesis of XZ-14515, XZ-14510, and XZ-14540

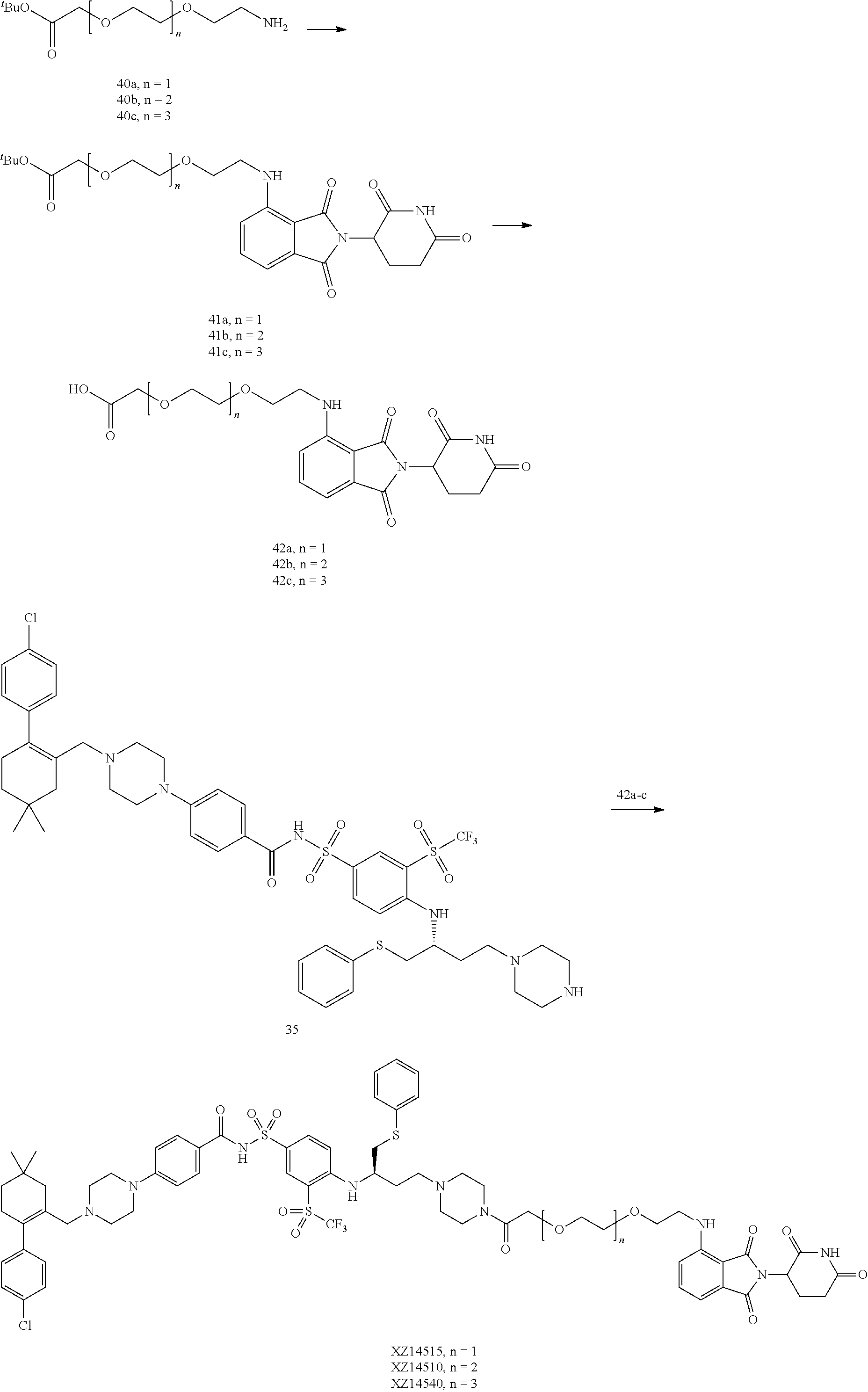

### General Procedure for the Preparation of 41a-c

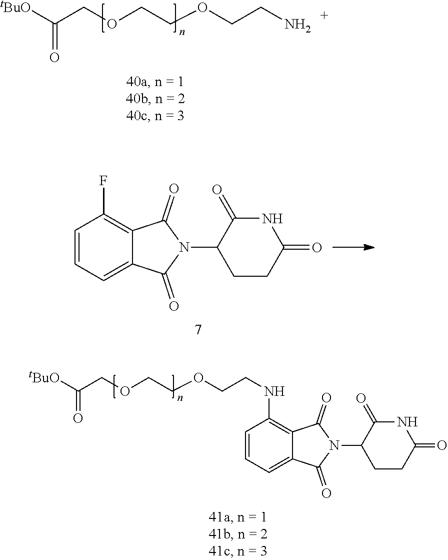

A mixture of compound 7 (1.0 equiv.), corresponding amine 40a-c (1.0 equiv.), and DIPEA (2.0 equiv.) in DMF were stirred at 90° C. overnight. The mixture was poured into water and extracted with EtOAc. The organic phase was washed with water×1, brine×1, dried over Na₂SO₄, filtered and evaporated to dryness. The crude product was purified by column chromatography using EtOAc and hexanes as eluents.

### General Procedure for the Preparation of 42a-c

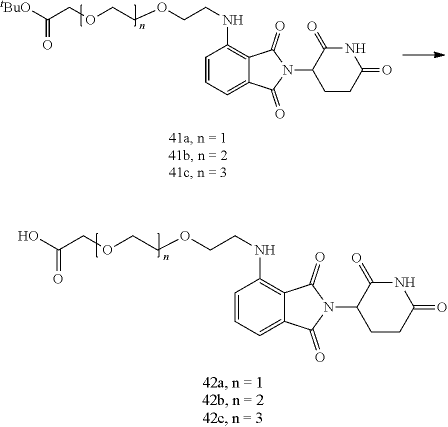

To a mixture of compound 41a, 41b, or 41c in DCM was added TFA. The mixture was stirred at room temperature overnight and the solvent was removed under reduced pressure. The crude product was washed with Et₂O to give the corresponding acid 42a, 42b, and 42c, respectively.

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(2-(2-(2-((2-(2,6-dioxopiperidin-3-yl)-1,3-dioxoisoindolin-4-yl)amino)ethoxy)ethoxy)acetyl)piperazin-1-yl)-1-(phenylthio)butan-2-14)amino)-3-((trifluoromethyl)sulfonyl)phenyl)sulfonyl)benzamide (XZ-14515)

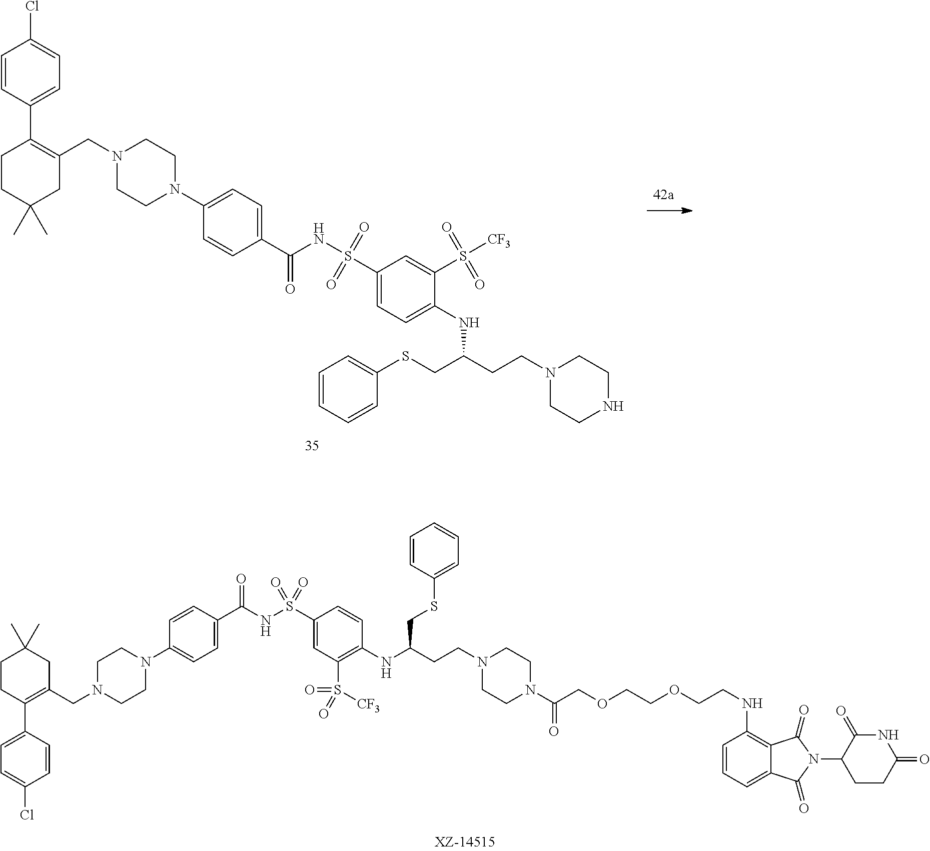 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)COCCOCCNc6cccc7c6C(=O)N(C6CCC(=O)NC6=O)C7=O)CC5)CSc5ccccc5)c(S(=O)(=O)C(F)(F)F)c4)cc3)CC2)C1]

A mixture of compound 35 (10 mg), 42a (5.5 mg), HATU (4 mg) and DIPEA (20 mg) in 3 mL DCM was stirred at room temperature for 1 hour. NH₄Cl (aq.) was then added to the mixture and the resulted mixture was extracted with DCM. The organic phase was washed with water×1, brine×1, dried over Na₂SO₄, filtered and evaporated to dryness. The crude product was purified by column chromatography using DCM and methanol as eluents to afford 6.6 mg pure XZ-14515. Yield 47%. ¹H NMR (400 MHz, CDCl₃ and CD₃OD) δ 9.11 (brs, 1H), 8.36 (s, 1H), 8.08 (d, J=9.1 Hz, 1H), 7.66 (d, J=8.2 Hz, 2H), 7.46 (t, J=7.8 Hz, 1H), 7.39-7.27 (m, 6H), 7.14-7.02 (m, 2H), 6.98 (d, J=8.3 Hz, 2H), 6.88 (d, J=8.5 Hz, 1H), 6.75 (d, J=8.6 Hz, 2H), 6.58 (d, J=5.8 Hz, 1H), 6.52-6.43 (brs, 1H), 4.91-4.81 (m, 1H), 4.26-4.15 (m, 2H), 3.94-3.81 (m, 1H), 3.70-3.33 (m, 12H), 3.31-3.22 (m, 4H), 3.12-2.93 (m, 2H), 2.89-2.55 (m, 5H), 2.49-2.00 (m, 16H), 1.74-1.70 (m, 1H), 1.51-1.45 (m, 2H), 0.98 (s, 6H) ppm.

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(2-(2-(2-(2-((2-(2,6-dioxopiperidin-3-yl)-1,3-dioxoisoindolin-4-yl)amino)ethoxy)ethoxy)ethoxy)acetyl)piperazin-1-yl)-1-(phenylthio)butan-2-yl)amino)-3-((trifluoromethyl)sulfonyl)phenyl)sulfonyl)benzamide (XZ-14510)

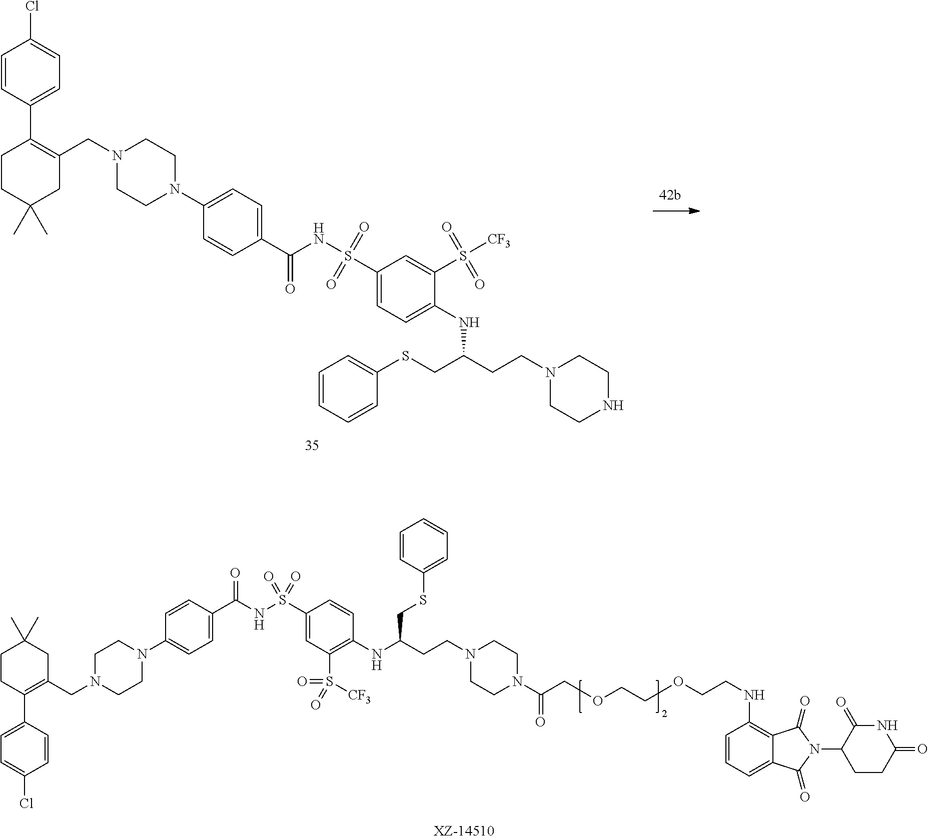

A mixture of compound 35 (10 mg), 42b (5 mg), HATU (4 mg) and DIPEA (20 mg) in 3 mL DCM was stirred at room temperature for 1 hour. NH₄Cl (aq) was then added to the mixture and the resulted mixture was extracted with DCM. The organic phase was washed with water×1, brine×1, dried over Na₂SO₄, filtered and evaporated to dryness. The crude product was purified by column chromatography using DCM and methanol as eluents to afford 6.2 mg pure XZ-14510. Yield 44%. ¹H NMR (400 MHz, CDCl₃ and CD₃OD) δ 8.33-8.28 (m, 1H), 8.07 (d, J=9.0 Hz, 1H), 7.78 (d, J=8.8 Hz, 2H), 7.54-7.45 (m, 1H), 7.41-7.37 (m, 1H), 7.33-7.20 (m, 5H), 7.09 (d, J=7.1 Hz, 1H), 7.06-6.98 (m, 3H), 6.94 (d, J=8.6 Hz, 1H), 6.79 (d, J=8.9 Hz, 2H), 6.61 (d, J=9.4 Hz, 1H), 5.00-4.86 (m, 1H), 4.21 (s, 2H), 3.93-3.84 (m, 1H), 3.70-3.39 (m, 16H), 3.32-3.25 (m, 4H), 3.12-3.00 (m, 2H), 2.93-2.71 (m, 5H), 2.46-2.24 (m, 12H), 2.09-2.00 (m, 4H), 1.75-1.63 (m, 1H), 1.47 (t, J=6.3 Hz, 2H), 0.99 (s, 6H) ppm.

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(14-((2-(2,6-dioxopiperidin-3-yl)-1,3-dioxoisoindolin-4-yl)amino)-3,6,9,12-tetraoxatetradecanoyl)piperazin-1-yl)-1-(phenylthio)butan-2-yl)amino)-3-((trifluoromethyl)sulfonyl)phenyl)sulfonyl)benzamide (XZ-14540)

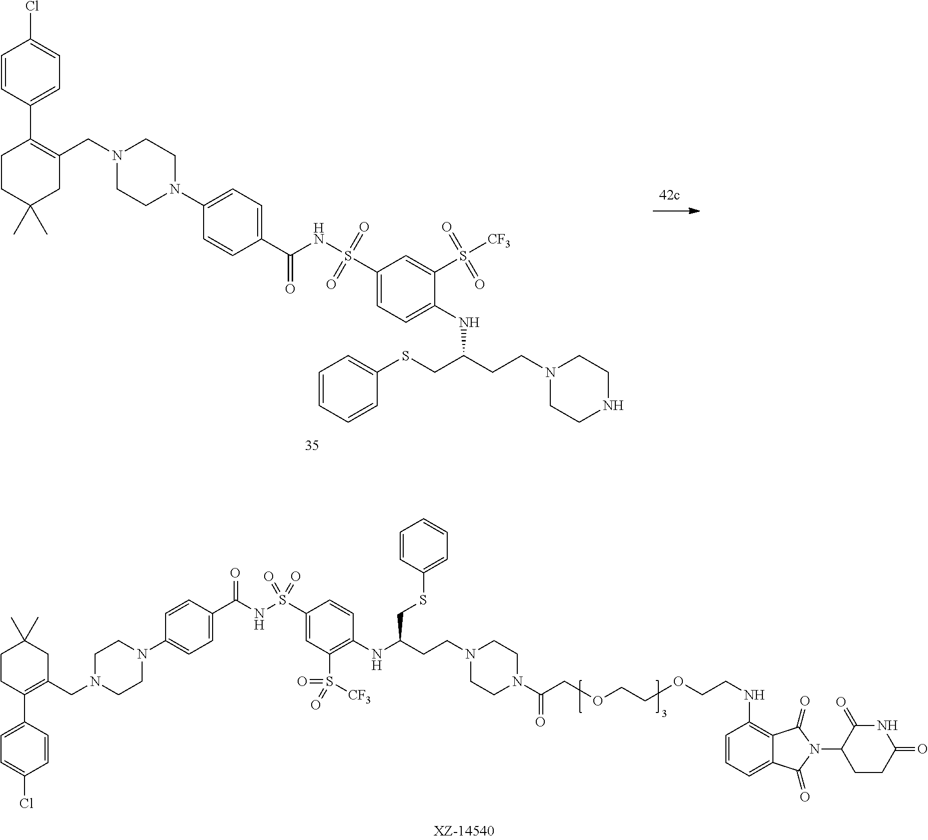

A mixture of compound 35 (12 mg), 42c (8.2 mg), HATU (5 mg) and DIPEA (30 mg) in 3 mL DCM was stirred at room temperature for 2 hours. NH₄Cl (aq.) was then added to the mixture and the resulted mixture was extracted with DCM. The organic phase was washed with water×1, brine×1, dried over Na₂SO₄, filtered and evaporated to dryness. The crude product was purified by column chromatography using DCM and methanol as eluents to afford 13.6 mg pure XZ14540. Yield 76%. ¹H NMR (400 MHz, CDCl₃ and CD₃OD) δ 8.81 (br s, 1H), 8.33 (s, 1H), 8.13-7.99 (m, 1H), 7.79-7.60 (m, 2H), 7.45 (t, J=7.8 Hz, 1H), 7.38-7.26 (m, 5H), 7.24-7.17 (m, 1H), 7.12-6.94 (m, 4H), 6.88 (d, J=8.6 Hz, 1H), 6.73 (d, J=8.8 Hz, 2H), 6.61-6.50 (m, 1H), 6.46 (br s, 1H), 4.95-4.84 (m, 1H), 4.16 (s, 2H), 3.88-3.80 (m, 1H), 3.72-3.39 (m, 20H), 3.32-3.25 (m, 4H), 3.12-3.01 (m, 2H), 2.93-2.71 (m, 5H), 2.46-2.24 (m, 12H), 2.09-2.00 (m, 4H), 1.75-1.63 (m, 1H), 1.47 (t, J=6.3 Hz, 2H), 0.96 (s, 6H) ppm.

### Example 11: Synthesis of XZ-15416, XZ-15405, and XZ-15418

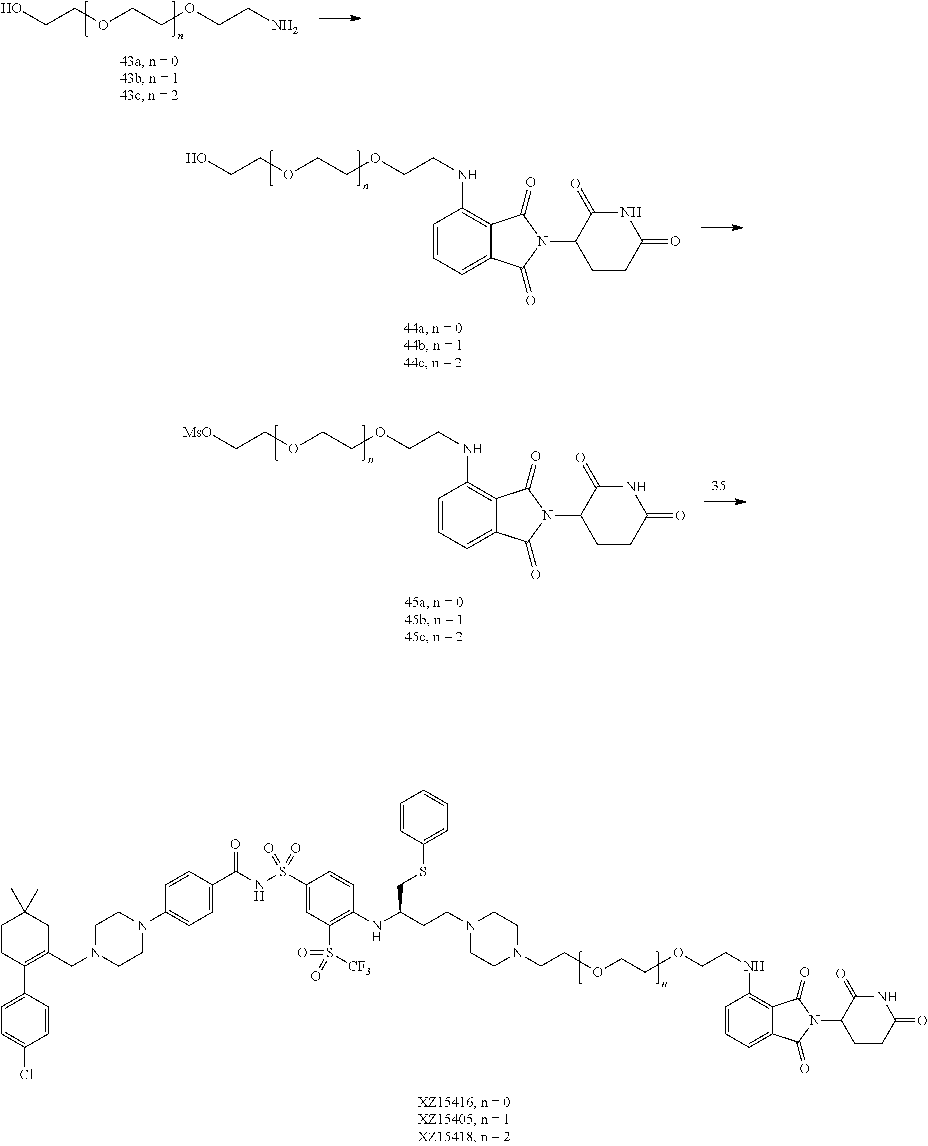

### General Procedure for the Preparation of 44a-c

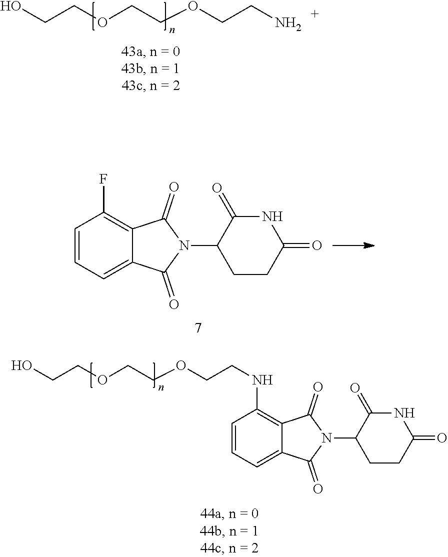

A mixture of compound 7 (1.0 equiv.), corresponding amine 43a-c (1.0 equiv.) and DIPEA (2.0 equiv.) in DMF were stirred at 90° C. overnight. The mixture was poured into water and extracted with EtOAc. The organic phase was washed with water×1, brine×1, dried over Na₂SO₄, filtered and evaporated to dryness. The crude product was purified by column chromatography using DCM and methanol as eluents.

### Preparation of 2-(2,6-Dioxopiperidin-3-yl)-4-((2-(2-hydroxyethoxy)ethyl)amino) isoindoline-1,3-dione (44a)

¹H NMR (400 MHz, CDCl₃) δ 8.25 (brs, 1H), 7.58-7.46 (m, 1H), 7.11 (d, J=7.1 Hz, 1H), 6.91 (d, J=8.5 Hz, 1H), 6.57 (t, J=5.4 Hz, 1H), 4.92 (dd, J=12.2, 5.3 Hz, 1H), 3.81-3.71 (m, 4H), 3.66-3.61 (m, 2H), 3.48 (dd, J=10.7, 5.3 Hz, 2H), 2.92-2.67 (m, 3H), 2.32 (brs, 1H), 2.18-2.07 (m, 1H) ppm.

### Preparation of 2-(2,6-Dioxopiperidin-3-yl)-4-((2-(2-(2-hydroxyethoxy)ethoxy)ethyl)amino) isoindoline-1,3-dione (44b)

¹H NMR (400 MHz, CDCl₃) δ 8.19 (brs, 1H), 7.55-7.44 (m, 1H), 7.10 (d, J=7.1 Hz, 1H), 6.91 (d, J=8.5 Hz, 1H), 6.57 (t, J=5.2 Hz, 1H), 4.91 (dd, J=12.0, 5.4 Hz, 1H), 3.85-3.65 (m, 8H), 3.64-3.59 (m, 2H), 3.51-3.43 (m, 2H), 2.92-2.68 (m, 3H), 2.57 (br s, 1H), 2.18-2.07 (m, 1H) ppm.

### Preparation of 2-(2,6-Dioxopiperidin-3-yl)-4-((2-(2-(2-(2-hydroxyethoxy)ethoxy)ethoxy) ethyl)amino)isoindoline-1,3-dione (44c)

¹H NMR (400 MHz, CDCl₃) δ 8.23 (br s, 1H), 7.58-7.40 (m, 1H), 7.10 (d, J=7.1 Hz, 1H), 6.92 (d, J=8.6 Hz, 1H), 6.52 (t, J=5.5 Hz, 1H), 4.92 (dd, J=12.0, 5.4 Hz, 1H), 3.77-3.65 (m, 12H), 3.63-3.58 (m, 2H), 3.52-3.44 (m, 2H), 3.00-2.59 (m, 4H), 2.24-2.04 (m, 1H) ppm.

### General Procedure for the Preparation of 45a-c

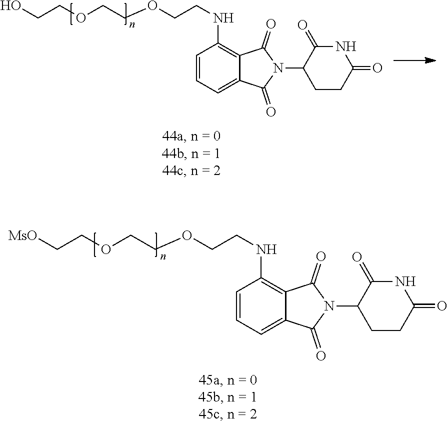

To a mixture of 44a, 44b, or 44c (1.0 equiv.), TEA (4.0 equiv.) in DCM was added MsCl (1.2 equiv.). The mixture was stirred at room temperature for 3 hours. Then the mixture was poured into water and extracted with EtOAc. The organic phase was washed with water×1, brine×1, dried over Na₂SO₄, filtered and evaporated to dryness. The crude product was purified by column chromatography using DCM and methanol as eluents.

### Preparation of 2-(2-((2-(2,6-Dioxopiperidin-3-yl)-1,3-dioxoisoindolin-4-yl)amino)ethoxy)ethyl methanesulfonate (45a)

¹H NMR (400 MHz, CDCl₃) δ 8.10 (br s, 1H), 7.63-7.44 (m, 1H), 7.12 (d, J=7.1 Hz, 1H), 6.93 (d, J=8.5 Hz, 1H), 6.49 (t, J=5.5 Hz, 1H), 4.91 (dd, J=12.1, 5.3 Hz, 1H), 4.48-4.35 (m, 2H), 3.86-3.66 (m, 4H), 3.58-3.41 (m, 2H), 3.13-2.69 (m, 6H), 2.23-2.05 (m, 1H) ppm.

### Preparation of 2-(2-(2-((2-(2,6-Dioxopiperidin-3-yl)-1,3-dioxoisoindolin-4-yl)amino)ethoxy)ethoxy)ethyl methanesulfonate (45b)

¹H NMR (400 MHz, CDCl₃) δ 8.14 (brs, 1H), 7.65-7.45 (m, 1H), 7.12 (d, J=7.1 Hz, 1H), 6.91 (d, J=8.5 Hz, 1H), 6.51 (t, J=5.1 Hz, 1H), 4.94 (dd, J=12.0, 5.3 Hz, 1H), 4.39 (dd, J=5.3, 3.7 Hz, 2H), 4.00-3.66 (m, 8H), 3.52-3.44 (m, 2H), 3.05 (s, 3H), 2.93-2.62 (m, 3H), 2.28-2.06 (m, 1H) ppm.

### Preparation of 2-(2-(2-(2-((2-(2,6-Dioxopiperidin-3-yl)-1,3-dioxoisoindolin-4-yl)amino)ethoxy)ethoxy) ethoxy)ethyl methanesulfonate (45c)

¹H NMR (400 MHz, CDCl₃) δ 8.20 (brs, 1H), 7.60-7.41 (m, 1H), 7.10 (d, J=7.1 Hz, 1H), 6.92 (d, J=8.5 Hz, 1H), 6.48 (t, J=5.6 Hz, 1H), 4.92 (dd, J=11.8, 5.4 Hz, 1H), 4.36 (dd, J=5.3, 3.7 Hz, 2H), 3.82-3.60 (m, 12H), 3.54-3.40 (m, 2H), 3.07 (s, 3H), 2.98-2.65 (m, 3H), 2.24-2.06 (m, 1H) ppm.

### Preparation of 4-(4-((4′-chloro-4,4-dimethyl-3,4,5,6-tetrahydro-[1,1′-biphenyl]-2-14)methyl)piperazin-1-yl)-N-((4-(((2R)-4-(4-(2-(2-((2-(2,6-dioxopiperidin-3-yl)-1,3-dioxoisoindolin-4-yl)amino)ethoxy)ethyl)piperazin-1-yl)-1-(phenylthio)butan-2-yl)amino)-3-((trifluoromethyl)sulfonyl)phenyl)sulfonyl)benzamide (XZ-15416)

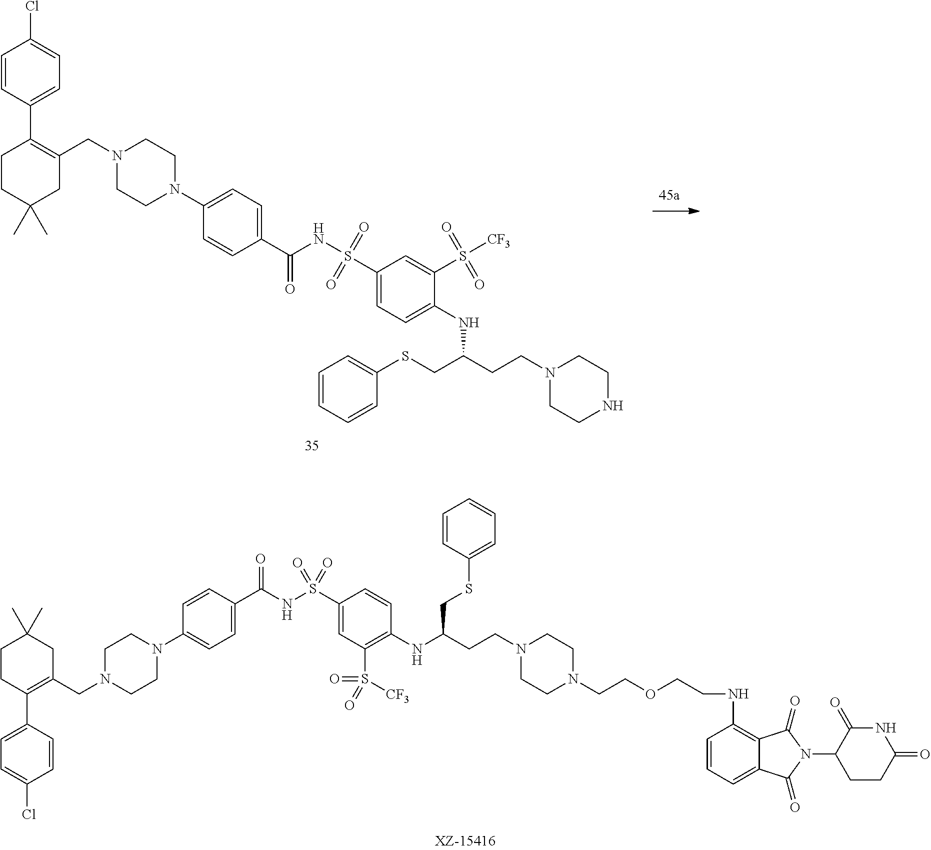 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(CCOCCNc6cccc7c6C(=O)N(C6CCC(=O)NC6=O)C7=O)CC5)CSc5ccccc5)c(S(=O)(=O)C(F)(F)F)c4)cc3)CC2)C1]

A mixture of compound 35 (25 mg), 45a (12 mg), DIPEA (60 μL) and NaI (1.6 mg) in 2 mL 1,4-dioxane was heated at 90° C. overnight. Then the mixture was poured into water and extracted with EtOAc. The organic phase was washed with water×1, NH₄Cl (aq)×1, brine×1, dried over Na₂SO₄, filtered and evaporated to dryness. The crude product was purified by column chromatography using DCM and methanol as eluents to afford 8.8 mg pure XZ-15416. Yield 26%. ¹H NMR (400 MHz, CDCl₃) δ 8.35 (s, 1H), 8.09-7.98 (m, 1H), 7.72 (d, J=8.7 Hz, 2H), 7.48 (t, J=7.9 Hz, 1H), 7.40-7.29 (m, 5H), 7.25-7.20 (m, 1H), 7.10 (d, J=7.1 Hz, 1H), 7.06-6.95 (m, 3H), 6.89 (d, J=8.4 Hz, 1H), 6.73 (d, J=9.1 Hz, 2H), 6.66-6.55 (m, 1H), 6.53-6.42 (m, 1H), 4.98-4.82 (m, 1H), 3.93-3.80 (m, 1H), 3.76-3.40 (m, 6H), 3.32-2.64 (m, 17H), 2.43-1.97 (m, 16H), 1.70-1.66 (m, 1H), 1.52-1.41 (m, 2H), 1.01-0.95 (m, 6H) ppm.

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(2-(2-(2-((2-(2,6-dioxopiperidin-3-yl)-1,3-dioxoisoindolin-4-yl)amino)ethoxy)ethoxy)ethyl)piperazin-1-yl)-1-(phenylthio)butan-2-14)amino)-3-((trifluoromethyl)sulfonyl)phenyl)sulfonyl)benzamide (XZ-15405)

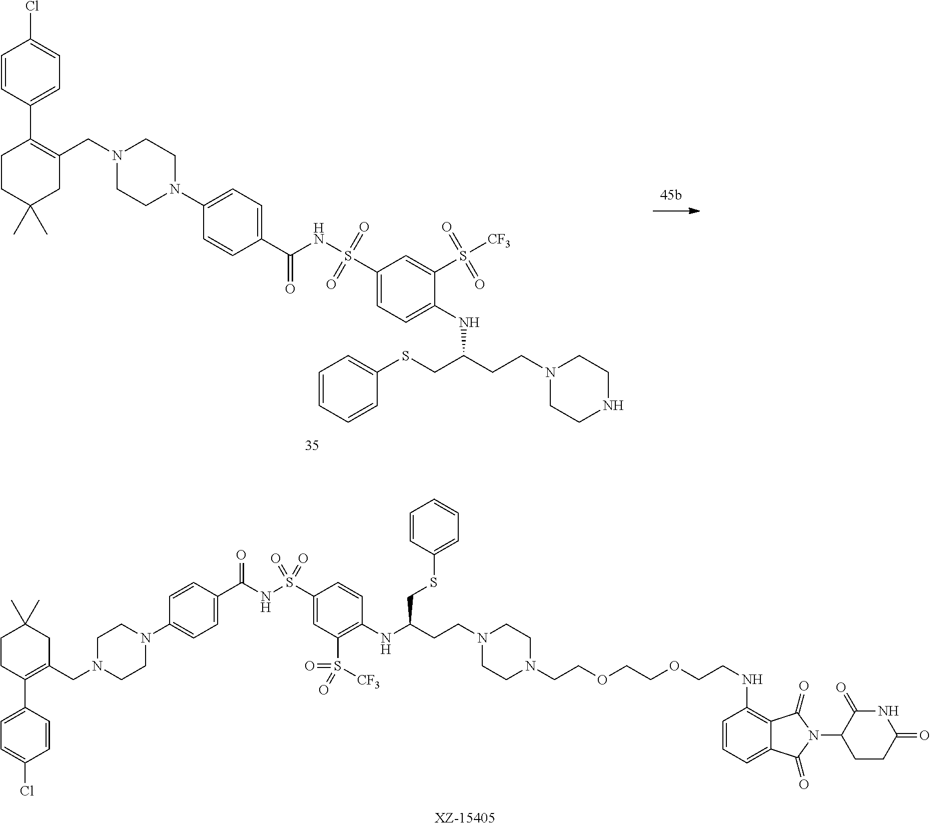 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(CCOCCOCCNc6cccc7c6C(=O)N(C6CCC(=O)NC6=O)C7=O)CC5)CSc5ccccc5)c(S(=O)(=O)C(F)(F)F)c4)cc3)CC2)C1]

A mixture of compound 35 (10 mg), 45b (6 mg), TEA (20 μL) and NaI (1.0 mg) in 2 mL 1,4-dioxane was heated at 80° C. overnight. The reaction mixture was then poured into water and extracted with EtOAc. The organic phase was washed with water×1, NH₄Cl (aq)×1, brine×1, dried over Na₂SO₄, filtered and evaporated to dryness. The crude product was purified by column chromatography using DCM and methanol as eluents to afford 5.8 mg pure XZ-15405. Yield 42%. ¹H NMR (400 MHz, CDCl₃) δ 8.33 (s, 1H), 8.02 (t, J=8.9 Hz, 1H), 7.76 (d, J=7.0 Hz, 2H), 7.52-7.46 (m, 1H), 7.41-7.33 (m, 2H), 7.32-7.27 (m, 3H), 7.25-7.21 (m, 1H), 7.10 (dd, J=7.1, 2.3 Hz, 1H), 7.04-6.92 (m, 3H), 6.88 (d, J=8.6 Hz, 1H), 6.75 (d, J=8.4 Hz, 2H), 6.56-6.40 (m, 2H), 4.96-4.73 (m, 1H), 3.86-3.40 (m, 11H), 3.33-2.51 (m, 17H), 2.50-1.79 (m, 16H), 1.74-1.60 (m, 1H), 1.48-1.37 (m, 2H), 0.95 (d, J=5.4 Hz, 6H) ppm.

### Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(4-(2-(2-(2-(2-((2-(2,6-dioxopiperidin-3-yl)-1,3-dioxoisoindolin-4-yl)amino)ethoxy)ethoxy)ethoxy)ethyl)piperazin-1-yl)-1-(phenylthio)butan-2-yl)amino)-3-((trifluoromethyl)sulfonyl)phenyl)sulfonyl)benzamide (XZ-15418)

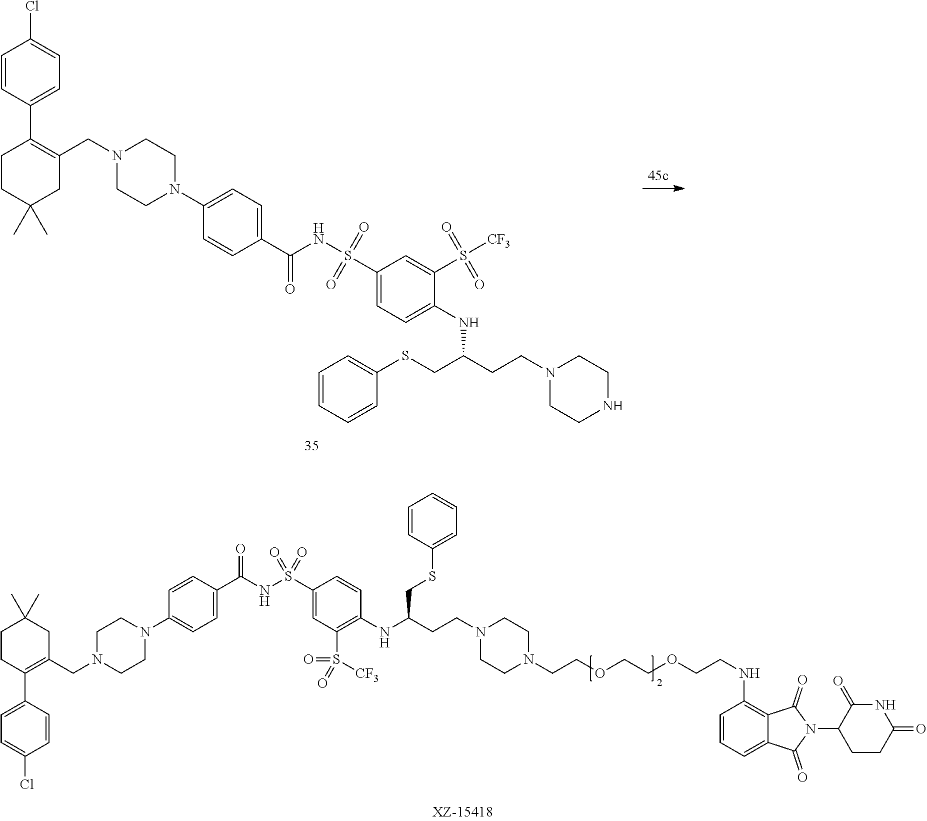

A mixture of compound 35 (42 mg), 45c (24 mg), DIPEA (100 μL) and NaI (3 mg) in 3 mL 1,4-dioxane was heated at 90° C. overnight. The reaction mixture was then poured into water and extracted with EtOAc. The organic phase was washed with water×1, NH₄Cl (aq)×1, brine×1, dried over Na₂SO₄, filtered and evaporated to dryness. The crude product was purified by column chromatography using DCM and methanol as eluents to afford 16.4 mg pure XZ-15418. Yield 25%. ¹H NMR (400 MHz, CDCl₃) δ 8.31 (s, 1H), 8.01 (d, J=8.9 Hz, 1H), 7.81 (d, J=8.5 Hz, 2H), 7.47 (t, J=7.8 Hz, 1H), 7.38-7.28 (m, 5H), 7.25-7.21 (m, 1H), 7.08 (d, J=7.1 Hz, 1H), 6.99 (d, J=8.3 Hz, 2H), 6.96-6.85 (m, 2H), 6.75 (d, J=8.7 Hz, 2H), 6.54-6.43 (m, 2H), 4.96-4.83 (m, 1H), 3.90-3.39 (m, 15H), 3.28-2.68 (m, 17H), 2.51-1.95 (m, 16H), 1.61-1.57 (m, 1H), 1.47-1.41 (m, 2H), 0.97-0.93 (m, 6H) ppm.

### Example 12: Synthesis of XZ-15421

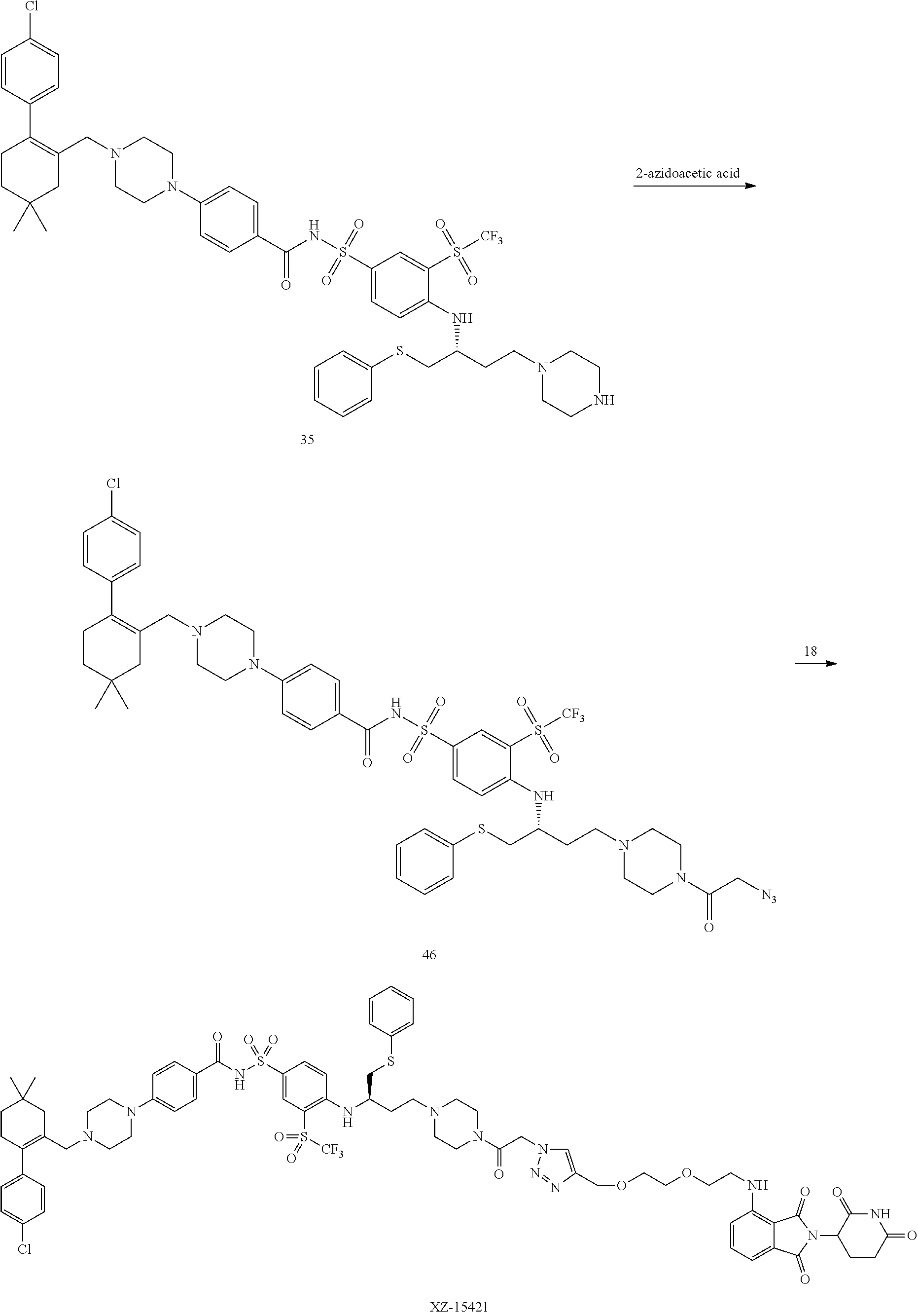 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)CN=[N+]=[N-])CC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)Cn6cc(COCCOCCNc7cccc8c7C(=O)N(C7CCC(=O)NC7=O)C8=O)nn6)CC5)CSc5ccccc5)c(S(=O)(=O)C(F)(F)F)c4)cc3)CC2)C1]

### Preparation of (R)—N-(4-(4-(4-(2-azidoacetyl)piperazin-1-yl)-1-(phenylthio)butan-2-ylamino)-3-(trifluoromethylsulfonyl)phenylsulfonyl)-4-(4-((2-(4-chlorophenyl)-5,5-dimethylcyclohex-1-enyl)methyl)piperazin-1-yl)benzamide (46)

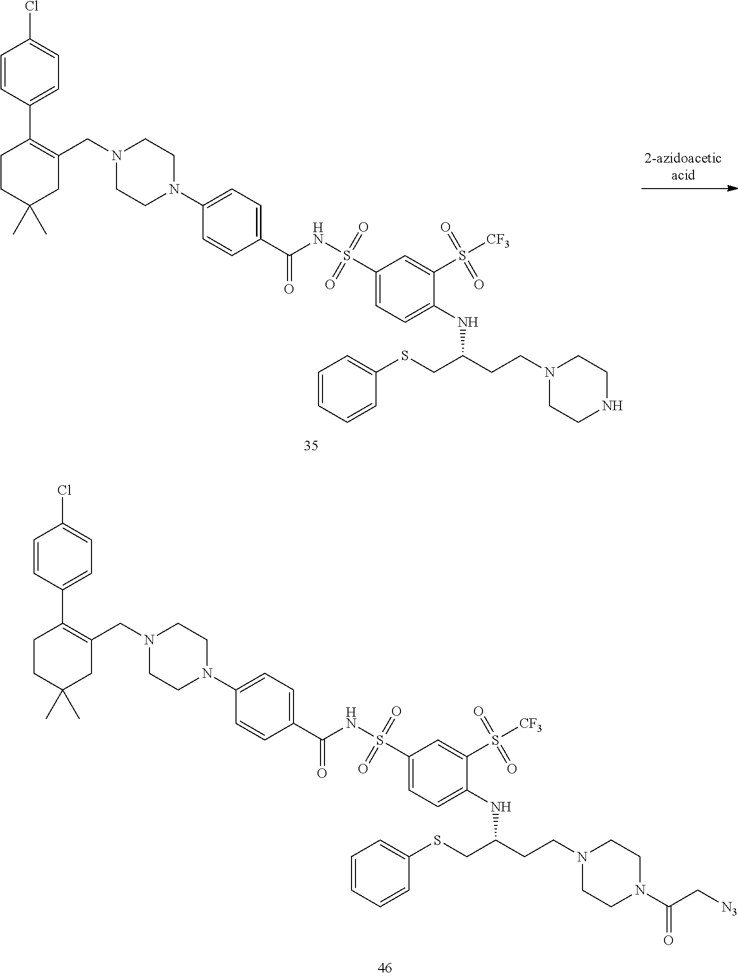 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCN(C(=O)CN=[N+]=[N-])CC5)CSc5ccccc5)c(S(=O)(=O)C(F)(F)F)c4)cc3)CC2)C1]

To a mixture of compound 35 (48 mg), 2-azidoacetic acid (8 mg), and DIPEA (13 μL) in 5 mL DCM was added HATU (21 mg). The mixture was stirred at room temperature for 3 hours. The solvent was removed under reduced pressure and the crude product was purified via column chromatography using DCM and methanol as eluents to afford 48 mg compound 46. Yield 87%. ¹H NMR (400 MHz, CDCl₃) δ 8.35 (d, J=1.9 Hz, 1H), 8.11 (dd, J=9.1, 1.8 Hz, 1H), 7.66 (d, J=8.8 Hz, 2H), 7.40-7.26 (m, 6H), 7.08 (d, J=8.4 Hz, 1H), 6.98 (d, J=8.3 Hz, 2H), 6.76 (d, J=8.8 Hz, 2H), 6.57 (d, J=9.4 Hz, 1H), 3.93-3.81 (m, 3H), 3.74-3.64 (m, 1H), 3.55-3.42 (m, 1H), 3.36-3.22 (m, 6H), 3.11 (dd, J=13.8, 4.8 Hz, 1H), 2.99 (dd, J=13.8, 7.5 Hz, 1H), 2.85 (s, 2H), 2.50-2.21 (m, 12H), 2.20-2.08 (m, 1H), 2.03-1.96 (m, 2H), 1.75-1.61 (m, 1H), 1.46 (t, J=6.4 Hz, 2H), 0.97 (s, 6H) ppm.

### Preparation of 4-(4-((2-(4-chlorophenyl)-5,5-dimethylcyclohex-1-enyl)methyl)piperazin-1-yl)-N-(4-((2R)-4-(4-(2-(4-((2-(2-(2-(2,6-dioxopiperidin-3-yl)-1,3-dioxoisoindolin-4-ylamino)ethoxy)ethoxy)methyl)-1H-1,2,3-triazol-1-yl)acetyl)piperazin-1-yl)-1-(phenylthio)butan-2-ylamino)-3-(trifluoromethylsulfonyl)phenylsulfonyl)benzamide (XZ-15421)

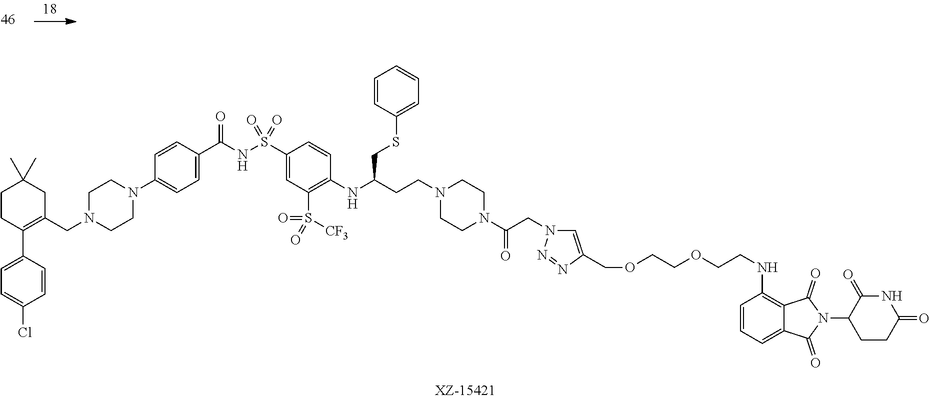 [SMILES: CC1(C)CCC(c2ccc(Cl)cc2)=C(CN2CCN(c3ccc(C(=O)NS(=O)(=O)c4ccc(N[C@H](CCN5CCN(C(=O)Cn6cc(COCCOCCNc7cccc8c7C(=O)N(C7CCC(=O)NC7=O)C8=O)nn6)CC5)CSc5ccccc5)c(S(=O)(=O)C(F)(F)F)c4)cc3)CC2)C1]

To a mixture of compound 46 (24.0 mg) and compound 18 (11.0 mg) in 2 mL t-BuOH-THF (1:1, v/v) under argon was added CuSO₄.5H₂O (1.15 mg) and sodium ascorbate (0.91 mg) in 0.3 mL water. The mixture was stirred at 50° C. for 2 hours and extracted with DCM. The organic phase was washed with brine×1, dried over Na₂SO₄, filtered and evaporated to dryness. The crude product was purified via column chromatography using DCM and methanol as eluents to afford 16.4 mg XZ-15421. Yield 50%. ¹H NMR (400 MHz, d₆-acetone) δ 9.91 (s, 1H), 8.34 (s, 1H), 8.10 (d, J=9.2 Hz, 1H), 7.92-7.78 (m, 3H), 7.66-7.55 (m, 1H), 7.48-7.20 (m, 7H), 7.17-7.00 (m, 6H), 6.90 (d, J=8.6 Hz, 2H), 6.70-6.54 (m, 1H), 5.31 (s, 2H), 5.14-5.05 (m, 1H), 4.63 (s, 2H), 4.33-4.25 (m, 1H), 3.79-3.20 (m, 20H), 3.02-2.83 (m, 5H), 2.58-2.11 (m, 14H), 1.87-1.82 (m, 1H), 1.48 (t, J=6.4 Hz, 2H), 1.00 (s, 6H) ppm.

### Example 13: Synthesis of XZ-14529

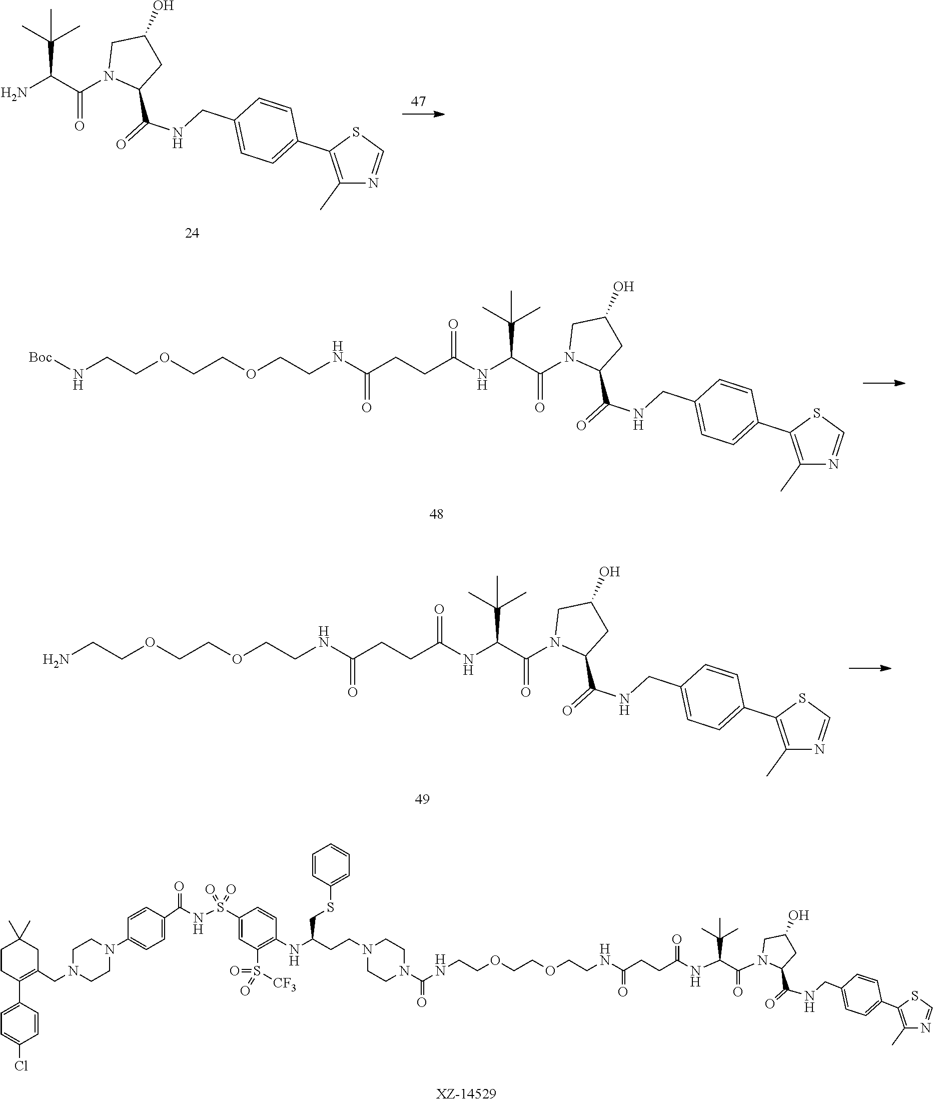 [SMILES: Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)CCC(=O)NCCOCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCC(=O)NCCOCCOCCN)C(C)(C)C)cc1 ; Cc1ncsc1-c1ccc(CNC(=O)[C@@H]2C[C@@H](O)CN2C(=O)[C@@H](NC(=O)CCC(=O)NCCOCCOCCNC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

### Preparation of 2,2-dimethyl-4,15-dioxo-3,8,11-trioxa-5,14-diazaoctadecan-18-oic acid (47)

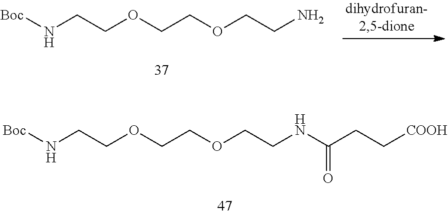 [SMILES: CC(C)(C)OC(=O)NCCOCCOCCN ; CC(C)(C)OC(=O)NCCOCCOCCNC(=O)CCC(=O)O]

A mixture of compound 37 (250 mg), dihydrofuran-2,5-dione (120 mg), and TEA (210 μL) in 10 mL DCM was stirred at room temperature overnight. The reaction mixture was then poured into water and extracted with DCM. The organic phase was washed with 1N HCl (aq.)×1, brine×1, dried over Na₂SO₄, filtered and evaporated to dryness to give 320 mg compound 47. Yield 92%. ¹H NMR (400 MHz, CDCl₃) δ 3.72-3.51 (m, 8H), 3.49-3.42 (m, 2H), 3.39-3.26 (m, 2H), 2.74-2.62 (m, 2H), 2.58-2.44 (m, 2H), 1.46 (s, 9H) ppm.

### Preparation of tert-butyl ((S)-15-((2S,4R)-4-hydroxy-2-((4-(4-methylthiazol-5-yl)benzyl)carbamoyl)pyrrolidine-1-carbonyl)-16,16-dimethyl-10,13-dioxo-3,6-dioxa-9,14-diazaheptadecyl)carbamate (48)

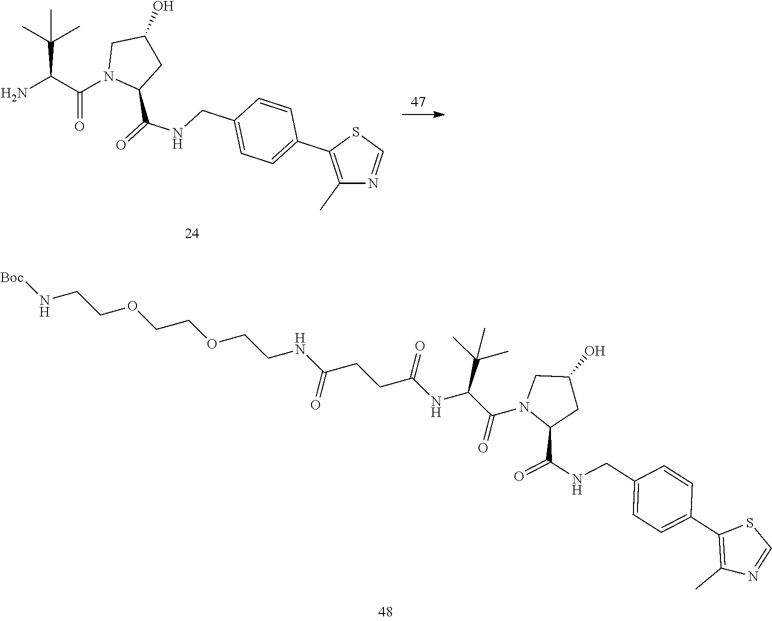 [SMILES: Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)CCC(=O)NCCOCCOCCNC(=O)OC(C)(C)C)C(C)(C)C)cc1]

To a mixture of compound 47 (100 mg), compound 24 (190 mg), and DIPEA (167 μL) in 10 mL DCM was added 116 mg HATU. The resulted mixture was stirred at room temperature for 2 hours before poured into water and extracted with DCM. The organic phase was washed with brine×1, dried over Na₂SO₄, filtered and evaporated to dryness to give 136 mg compound 48. Yield 62%. ¹H NMR (400 MHz, CDCl₃) δ 8.67 (s, 1H), 7.53 (br s, 1H), 7.40-7.29 (m, 4H), 6.97 (br s, 1H), 6.53 (br s, 1H), 5.12 (br s, 1H), 4.72 (t, J=8.0 Hz, 1H), 4.62-4.44 (m, 3H), 4.33 (dd, J=15.0, 5.3 Hz, 1H), 4.12-3.91 (m, 1H), 3.65-3.46 (m, 9H), 3.45-3.22 (m, 4H), 2.55-2.37 (m, 8H), 2.21-2.09 (m, 1H), 1.43 (s, 9H), 0.91 (s, 9H) ppm.

### Preparation of N1-(2-(2-(2-aminoethoxy)ethoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-14)succinamide (49)

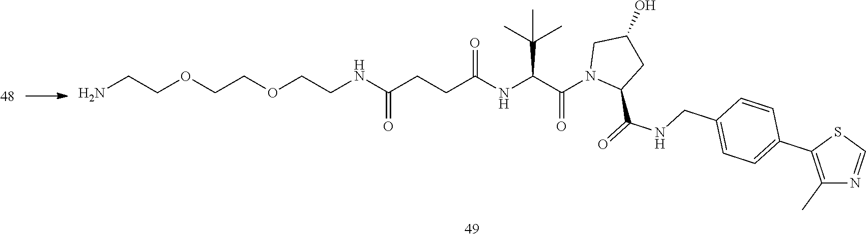 [SMILES: Cc1ncsc1-c1ccc(CNC(=O)[C@@H]2C[C@@H](O)CN2C(=O)[C@@H](NC(=O)CCC(=O)NCCOCCOCCN)C(C)(C)C)cc1]

To a mixture of compound 48 (100 mg) in 10 mL DCM was added TFA (440 μL). The reaction was stirred at room temperature for 2 days. Then it was neutralized with NaHCO₃ (aq) and extracted with DCM. The organic phase was washed with brine×1, dried over Na₂SO₄, filtered and evaporated to dryness to give the crude product which can be used in the next step without further purification. ¹H NMR (400 MHz, CDCl₃) δ 8.67 (s, 1H), 7.67-7.58 (m, 1H), 7.39-7.28 (m, 5H), 6.94 (d, J=8.2 Hz, 1H), 4.70 (t, J=8.3 Hz, 1H), 4.56-4.30 (m, 4H), 4.03 (d, J=10.9 Hz, 1H), 3.64-3.49 (m, 9H), 3.43-3.34 (m, 2H), 2.89 (t, J=4.9 Hz, 2H), 2.57-2.36 (m, 8H), 2.20-2.13 (m, 1H), 0.95 (s, 9H) ppm.

### Preparation of N1-(2-(2-(2-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carboxamido)ethoxy)ethoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide (XZ-14529)

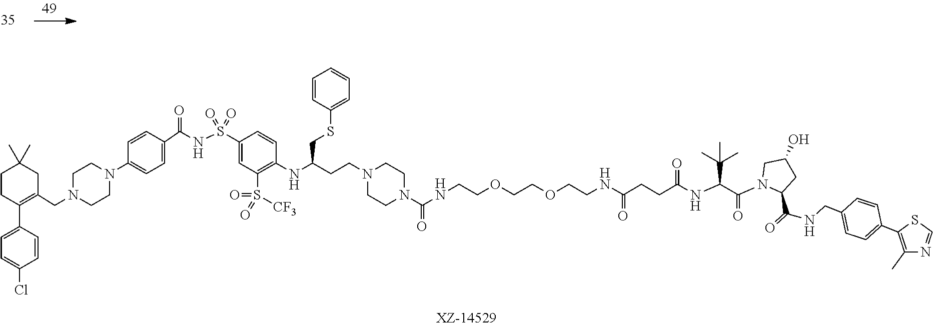 [SMILES: Cc1ncsc1-c1ccc(CNC(=O)[C@@H]2C[C@@H](O)CN2C(=O)[C@@H](NC(=O)CCC(=O)NCCOCCOCCNC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

A mixture of compound 49 (26 mg) and CDI (7.7 mg) in 2 mL THF was stirred at room temperature for 1 hour. Compound 35 (14.6 mg) and DIPEA (0.05 mL) were then added. The mixture was stirred overnight and quenched by the addition of NH₄Cl (aq.), extracted with DCM and the organic phase was washed with water×1, brine×1, dried over Na₂SO₄, filtered and evaporated to dryness. The crude product was purified by column chromatography using DCM and methanol as eluents to afford 21.1 mg compound XZ-14529. Yield 85%. ¹H NMR (400 MHz, CDCl₃ and CD₃OD) δ 8.70 (s, 1H), 8.32 (s, 1H), 8.03 (d, J=8.8 Hz, 1H), 7.95-7.76 (m, 3H), 7.50-7.38 (m, 2H), 7.37-7.34 (m, 4H), 7.32-7.17 (m, 5H), 7.06-6.95 (m, 3H), 6.78 (d, J=8.9 Hz, 2H), 6.65 (d, J=9.4 Hz, 1H), 5.87-5.72 (m, 1H), 4.66-4.47 (m, 4H), 4.45-4.33 (m, 1H), 4.01-3.22 (m, 23H), 3.16-3.03 (m, 2H), 2.84 (s, 2H), 2.52-1.98 (m, 24H), 1.75-1.63 (m, 1H), 1.48 (t, J=6.3 Hz, 2H), 0.99 (m, 15H) ppm.

### Example 14: Synthesis of XZ-14523

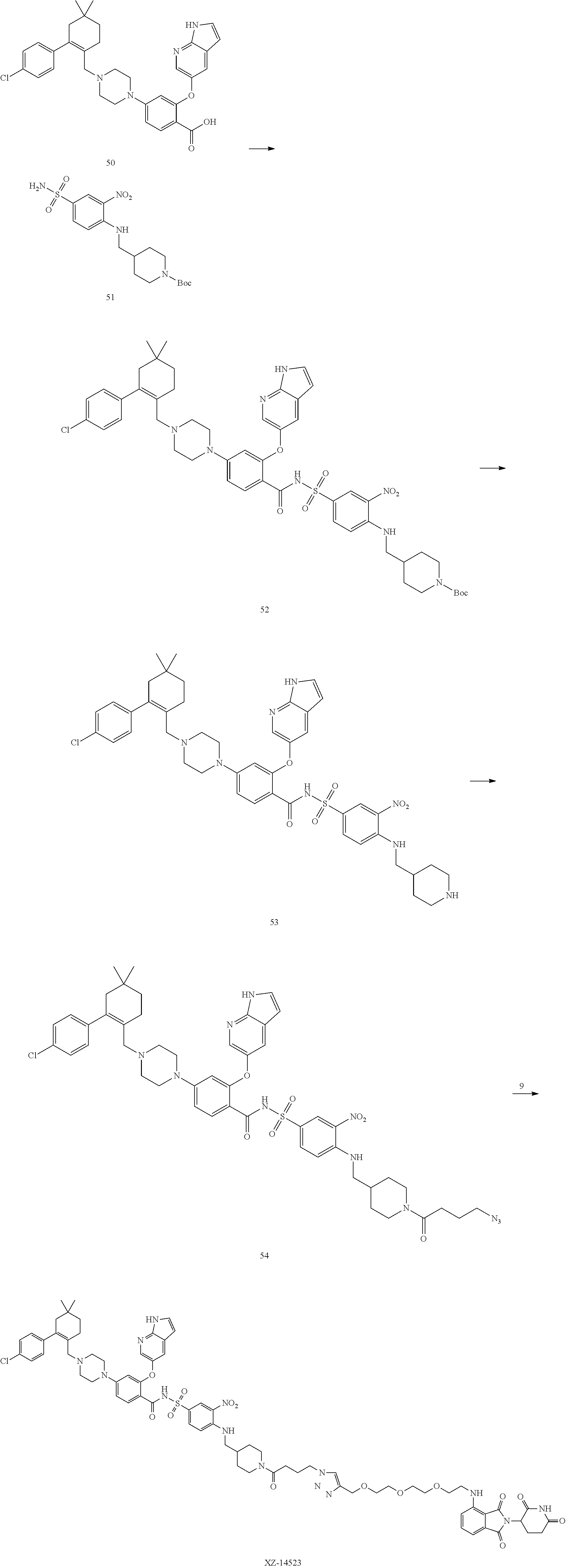

### Preparation of tert-butyl 4-((4-(N-(2-(1H-pyrrolo[2,3-b]pyridin-5-yloxy)-4-(4-((2-(4-chlorophenyl)-4,4-dimethylcyclohex-1-enyl)methyl)piperazin-1-yl)benzoyl)sulfamoyl)-2-nitrophenylamino)methyl)piperidine-1-carboxylate (52)

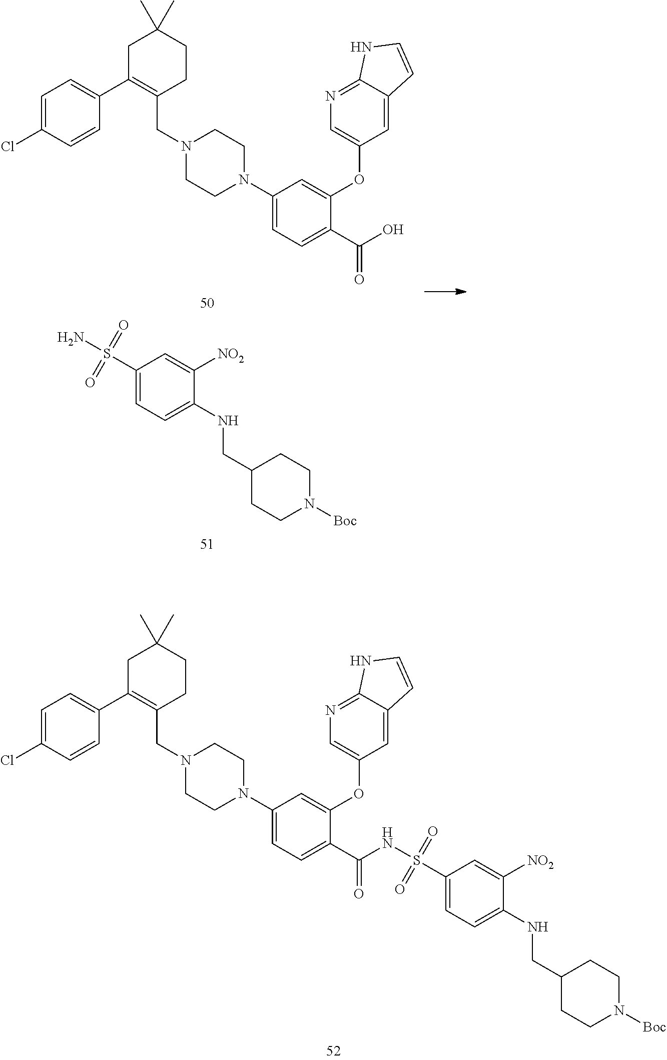 [SMILES: CC1(C)CCC(CN2CCN(c3ccc(C(=O)O)c(Oc4cnc5[nH]ccc5c4)c3)CC2)=C(c2ccc(Cl)cc2)C1 ; CC(C)(C)OC(=O)N1CCC(CNc2ccc(S(N)(=O)=O)cc2[N+](=O)[O-])CC1 ; CC1(C)CCC(CN2CCN(c3ccc(C(=O)NS(=O)(=O)c4ccc(NCC5CCN(C(=O)OC(C)(C)C)CC5)c([N+](=O)[O-])c4)c(Oc4cnc5[nH]ccc5c4)c3)CC2)=C(c2ccc(Cl)cc2)C1]

A mixture of compound 50 (571 mg), 51 (415 mg), DMAP (244 mg), EDCl (250 mg), and TEA (280 μL) in 20 mL DCM was stirred at room temperature overnight. The solvent was removed under reduced pressure and the residue was purified via column chromatography using DCM and methanol as eluents to give 758 mg pure product as yellow solid. Yield 79%. ¹H NMR (400 MHz, CDCl₃) δ 10.14 (brs, 1H), 9.72 (brs, 1H), 8.89 (d, J=2.2 Hz, 1H), 8.52 (t, J=5.4 Hz, 1H), 8.21 (d, J=2.5 Hz, 1H), 8.16 (dd, J=9.2, 2.1 Hz, 1H), 7.95 (d, J=9.1 Hz, 1H), 7.71 (d, J=2.5 Hz, 1H), 7.53-7.43 (m, 1H), 7.22 (d, J=8.4 Hz, 2H), 6.94-6.83 (m, 3H), 6.60-6.47 (m, 2H), 5.98 (d, J=2.1 Hz, 1H), 4.27-4.13 (m, 2H), 3.32-3.20 (m, 2H), 3.13-3.01 (m, 4H), 2.83-2.65 (m, 4H), 2.26-2.10 (m, 6H), 1.96 (s, 2H), 1.92-1.74 (m, 3H), 1.47 (s, 9H), 1.40 (t, J=6.4 Hz, 2H), 1.25-1.18 (m, 2H), 0.93 (s, 6H) ppm.

### Preparation of 2-(1H-pyrrolo[2,3-b]pyridin-5-yloxy)-4-(4-((2-(4-chlorophenyl)-4,4-dimethylcyclohex-1-enyl)methyl)piperazin-1-yl)-N-(3-nitro-4-(piperidin-4-ylmethylamino)phenylsulfonyl)benzamide (53)

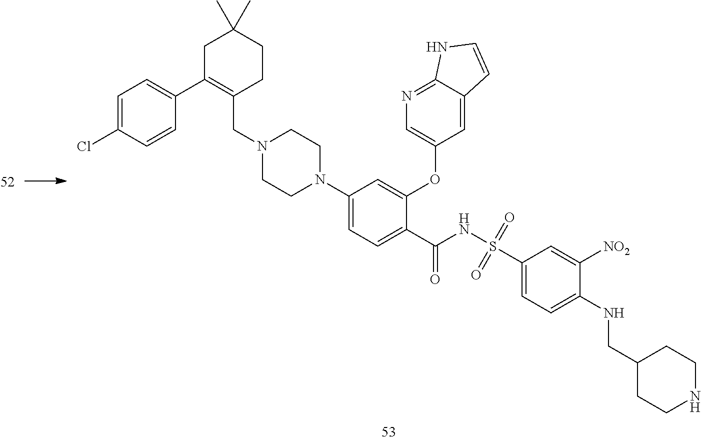 [SMILES: CC1(C)CCC(CN2CCN(c3ccc(C(=O)NS(=O)(=O)c4ccc(NCC5CCNCC5)c([N+](=O)[O-])c4)c(Oc4cnc5[nH]ccc5c4)c3)CC2)=C(c2ccc(Cl)cc2)C1]

To a solution of compound 52 (578 mg) in 20 mL DCM was added TFA (440 μL). The reaction mixture was stirred at room temperature overnight. Solvent was removed under reduced pressure and Et₂O was added to the residue. The precipitated solid was collected by filtration and can be used directly in the next step without further purification. ¹H NMR (400 MHz, d₆-DMSO) δ 11.88-11.54 (m, 2H), 9.34 (br s, 1H), 8.66 (t, J=6.1 Hz, 1H), 8.59-8.45 (m, 2H), 8.29-8.08 (m, 1H), 8.01 (d, J=2.6 Hz, 1H), 7.80 (dd, J=9.2, 2.2 Hz, 1H), 7.57-7.45 (m, 3H), 7.36 (d, J=8.4 Hz, 2H), 7.12 (d, J=9.4 Hz, 1H), 7.05 (d, J=8.3 Hz, 2H), 6.69 (dd, J=9.0, 2.0 Hz, 1H), 6.37 (dd, J=3.3, 1.9 Hz, 1H), 6.22 (s, 1H), 3.84-3.44 (m, 4H), 3.39-3.15 (m, 6H), 3.10-2.60 (m, 6H), 2.22-2.10 (m, 2H), 2.04-1.76 (m, 5H), 1.49-1.26 (m, 4H), 0.91 (s, 6H) ppm.

### Preparation of 2-((1H-pyrrolo[2,3-b]pyridin-5-yl)oxy)-N-((4-(((1-(4-azidobutanoyl)piperidin-4-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)benzamide (54)

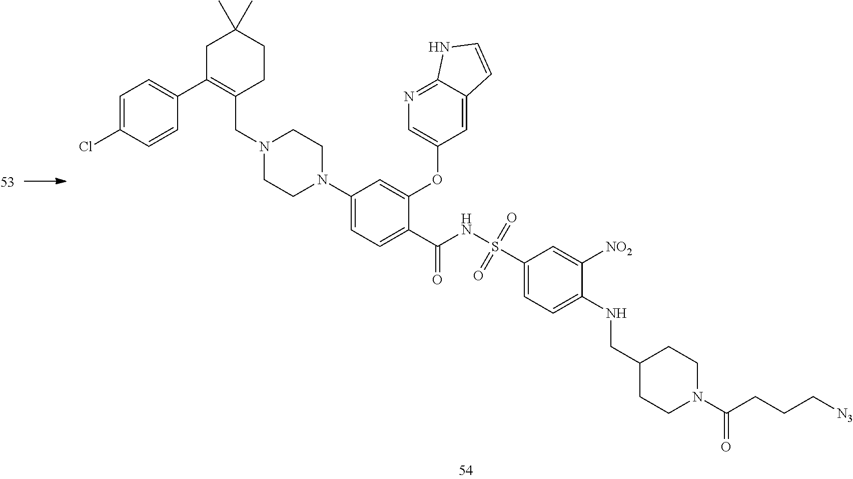 [SMILES: CC1(C)CCC(CN2CCN(c3ccc(C(=O)NS(=O)(=O)c4ccc(NCC5CCN(C(=O)CCCN=[N+]=[N-])CC5)c([N+](=O)[O-])c4)c(Oc4cnc5[nH]ccc5c4)c3)CC2)=C(c2ccc(Cl)cc2)C1]

To a solution of compound 53 (60 mg), 4-azidobutanoic acid (7 mg), and DIPEA (42 μL) in 5 mL DCM was added HATU (20 mg). The resulted mixture was stirred at room temperature for 2 hours. Solvent was removed under reduced pressure and the crude product was purified via column chromatography using DCM and methanol as eluents to afford 46 mg compound 54. Yield 94%. ¹H NMR (400 MHz, CDCl₃) δ 10.14 (br s, 1H), 9.46 (s, 1H), 8.90 (d, J=2.2 Hz, 1H), 8.52 (t, J=5.4 Hz, 1H), 8.27-8.10 (m, 2H), 7.95 (d, J=9.1 Hz, 1H), 7.71 (d, J=2.5 Hz, 1H), 7.51-7.44 (m, 1H), 7.22 (d, J=8.4 Hz, 2H), 7.01-6.76 (m, 3H), 6.62-6.43 (m, 2H), 5.98 (d, J=2.1 Hz, 1H), 4.72 (d, J=13.5 Hz, 1H), 3.94 (d, J=13.8 Hz, 1H), 3.40 (t, J=6.4 Hz, 2H), 3.34-3.20 (m, 2H), 3.13-2.98 (m, 5H), 2.74 (s, 2H), 2.58 (t, J=11.7 Hz, 1H), 2.44 (t, J=7.2 Hz, 2H), 2.29-2.09 (m, 6H), 2.04-1.82 (m, 7H), 1.40 (t, J=6.4 Hz, 2H), 1.28-1.18 (m, 2H), 0.93 (s, 6H) ppm.

### Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(((1-(4-(4-((2-(2-(2-((2-(2,6-dioxopiperidin-3-yl)-1,3-dioxoisoindolin-4-14)amino)ethoxy)ethoxy)ethoxy)methyl)-1H-1,2,3-triazol-1-yl)butanoyl)piperidin-4-yl)methyl)amino)-3-nitrophenyl)sulfonyl)benzamide (XZ-14523)

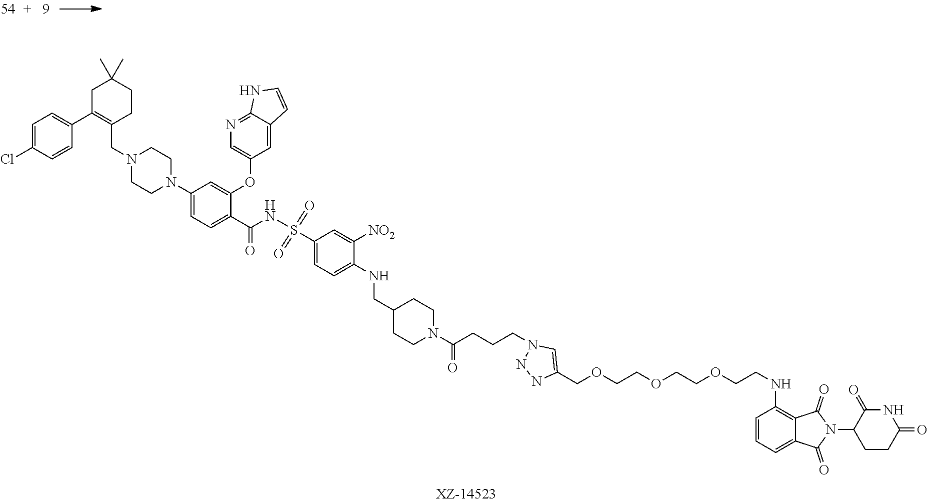 [SMILES: CC1(C)CCC(CN2CCN(c3ccc(C(=O)NS(=O)(=O)c4ccc(NCC5CCN(C(=O)CCCN6C=C(COCCOCCOCCNc7cccc8c7C(=O)N(C7CCC(=O)NC7=O)C8=O)NN6)CC5)c([N+](=O)[O-])c4)c(Oc4cnc5[nH]ccc5c4)c3)CC2)=C(c2ccc(Cl)cc2)C1]

To a mixture of compound 54 (20.0 mg), compound 9 (10.0 mg) in 2 mL t-BuOH-THF (1:1, v/v) under argon was added CuSO₄.5H₂O (1.0 mg) and sodium ascorbate (0.8 mg) in 0.3 mL water. The mixture was stirred at 55° C. for 3 hours and extracted with DCM. The organic phase was washed with brine×1, dried over Na₂SO₄, filtered and evaporated to dryness. The crude product was purified via column chromatography using DCM and methanol as eluents to afford 23.2 mg XZ-15423. Yield 82%. ¹H NMR (400 MHz, CDCl₃) δ 10.07 (br s, 1H), 9.88 (br s, 1H), 9.61 (br s, 1H), 8.89 (d, J=2.2 Hz, 1H), 8.64-8.46 (m, 1H), 8.19 (d, J=2.5 Hz, 1H), 8.07 (d, J=9.2 Hz, 1H), 7.93 (d, J=9.1 Hz, 1H), 7.69 (d, J=2.5 Hz, 1H), 7.62 (s, 1H), 7.52-7.42 (m, 2H), 7.23 (d, J=8.3 Hz, 2H), 7.08 (d, J=7.1 Hz, 1H), 6.96-6.83 (m, 4H), 6.61-6.39 (m, 3H), 5.98 (d, J=2.0 Hz, 1H), 4.98-4.90 (m, 1H), 4.76-4.63 (m, 3H), 4.53-4.37 (m, 2H), 3.88-3.62 (m, 11H), 3.50-3.41 (m, 2H), 3.33-3.22 (m, 2H), 3.10-3.02 (m, 4H), 3.02-2.69 (m, 6H), 2.55 (t, J=11.8 Hz, 1H), 2.38-2.11 (m, 11H), 2.00-1.80 (m, 5H), 1.41 (t, J=7.3 Hz, 2H), 1.27-1.21 (m, 2H), 0.93 (s, 6H) ppm.

### Example 15: Synthesis of XZ-14522

### Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(((1-(2-(2-(2-(2-((2-(2,6-dioxopiperidin-3-yl)-1,3-dioxoisoindolin-4-yl)amino)ethoxy)ethoxy)ethoxy)acetyl)piperidin-4-yl)methyl)amino)-3-nitrophenyl)sulfonyl)benzamide (XZ-14522)

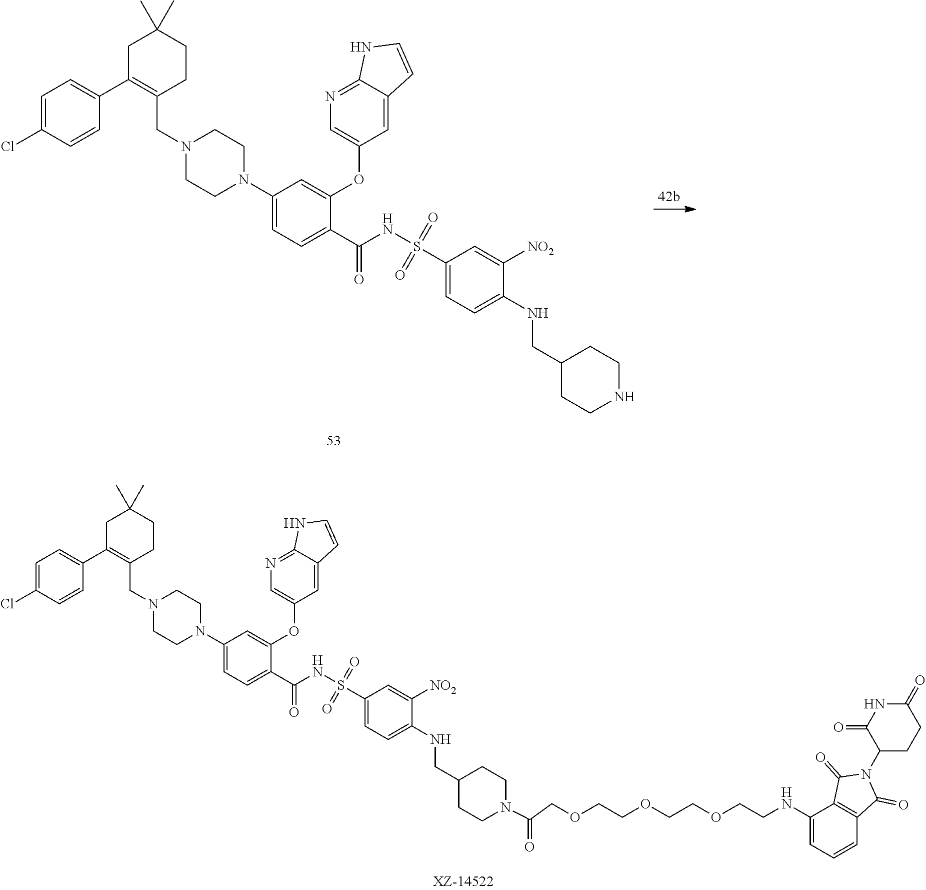 [SMILES: CC1(C)CCC(CN2CCN(c3ccc(C(=O)NS(=O)(=O)c4ccc(NCC5CCNCC5)c([N+](=O)[O-])c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCC5CCN(C(=O)COCCOCCOCCNc6cccc7c6C(=O)N(C6CCC(=O)NC6=O)C7=O)CC5)c([N+](=O)[O-])c4)c(Oc4cnc5[nH]ccc5c4)c3)CC2)=C(c2ccc(Cl)cc2)C1]

A solution of compound 53 (12 mg), compound 42b (5 mg), HATU (4 mg), and DIPEA (20 mg) in 3 mL DCM was stirred at room temperature for 2 hours. NH₄Cl (aq) was then added and extracted with DCM. The organic phase was washed with water×1, brine×1, dried over Na₂SO₄, filtered and evaporated to dryness. The crude product was purified by column chromatography using DCM and methanol as eluents to afford 10.8 mg pure XZ-14522. Yield 82%. ¹H NMR (400 MHz, CDCl₃) δ 10.24 (d, J=6.9 Hz, 1H), 10.10 (br s, 1H), 9.82 (br s, 1H), 8.87 (s, 1H), 8.49 (t, J=5.3 Hz, 1H), 8.16 (d, J=2.1 Hz, 1H), 8.07 (d, J=9.0 Hz, 1H), 7.92 (d, J=9.1 Hz, 1H), 7.75-7.62 (m, 1H), 7.54-7.41 (m, 2H), 7.23 (d, J=8.3 Hz, 2H), 7.08 (d, J=7.1 Hz, 1H), 6.99-6.78 (m, 4H), 6.60-6.43 (m, 3H), 6.04-5.81 (m, 1H), 5.01-4.85 (m, 1H), 4.63 (d, J=12.5 Hz, 1H), 4.39-4.00 (m, 3H), 3.75-3.64 (m, 10H), 3.52-3.40 (m, 2H), 3.31-2.67 (m, 12H), 2.59 (t, J=12.4 Hz, 1H), 2.27-1.81 (m, 12H), 1.46-1.38 (m, 2H), 1.26-1.24 (m, 2H), 0.93 (s, 6H) ppm.

### Example 16: Synthesis of XZ-14528

### Preparation of N1-(2-(2-(2-(4-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-nitrophenyl)amino)methyl)piperidine-1-carboxamido)ethoxy)ethoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide (XZ-14528)

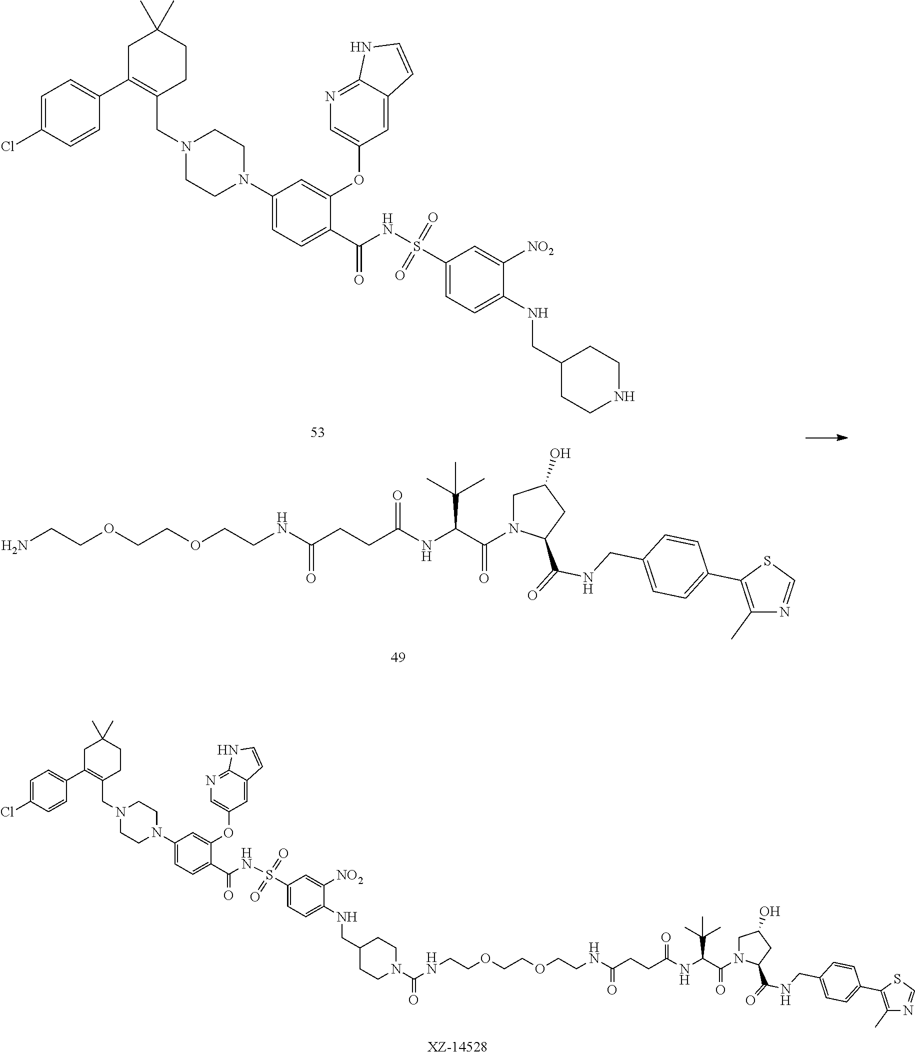 [SMILES: CC1(C)CCC(CN2CCN(c3ccc(C(=O)NS(=O)(=O)c4ccc(NCC5CCNCC5)c([N+](=O)[O-])c4)c(Oc4cnc5[nH]ccc5c4)c3)CC2)=C(c2ccc(Cl)cc2)C1 ; Cc1ncsc1-c1ccc(CNC(=O)[C@@H]2C[C@@H](O)CN2C(=O)[C@@H](NC(=O)CCC(=O)NCCOCCOCCN)C(C)(C)C)cc1 ; Cc1ncsc1-c1ccc(CNC(=O)[C@@H]2C[C@@H](O)CN2C(=O)[C@@H](NC(=O)CCC(=O)NCCOCCOCCNC(=O)N2CCC(CNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3[N+](=O)[O-])CC2)C(C)(C)C)cc1]

A mixture of compound 49 (26 mg) and CDI (7.7 mg) in 2 mL THF was stirred at room temperature for 1 hour. Compound 53 (18.1 mg) and DIPEA (0.05 mL) were then added. The mixture was stirred overnight and quenched by the addition of NH₄Cl (aq.), extracted with DCM and the organic phase was washed with water×1, brine×1, dried over Na₂SO₄, filtered and evaporated to dryness. The crude product was purified by column chromatography using DCM and methanol as eluents to afford 16.8 mg compound XZ-14528. Yield 72%. ¹H NMR (400 MHz, CDCl₃) δ 9.49 (s, 1H), 8.85 (d, J=2.2 Hz, 1H), 8.67 (s, 1H), 8.57-8.43 (m, 1H), 8.15 (d, J=2.5 Hz, 1H), 7.99 (d, J=9.2 Hz, 1H), 7.92 (d, J=9.1 Hz, 1H), 7.62 (d, J=2.4 Hz, 1H), 7.46-7.40 (m, 2H), 7.38-7.32 (m, 4H), 7.23 (d, J=8.3 Hz, 2H), 6.97-6.88 (m, 3H), 6.80 (d, J=9.4 Hz, 1H), 6.62-6.47 (m, 3H), 6.02 (d, J=1.7 Hz, 1H), 5.24-5.17 (m, 1H), 4.73 (t, J=8.0 Hz, 1H), 4.63-4.47 (m, 3H), 4.35 (dd, J=15.0, 5.2 Hz, 1H), 4.10-3.89 (m, 4H), 3.64-3.47 (m, 12H), 3.46-3.37 (m, 4H), 3.24 (t, J=6.1 Hz, 2H), 3.15-3.01 (m, 4H), 2.87-2.71 (m, 4H), 2.60-2.38 (m, 8H), 2.20-1.76 (m, 8H), 1.42-1.34 (m, 2H), 1.28-1.24 (m, 2H), 0.98-0.92 (m, 15H) ppm.

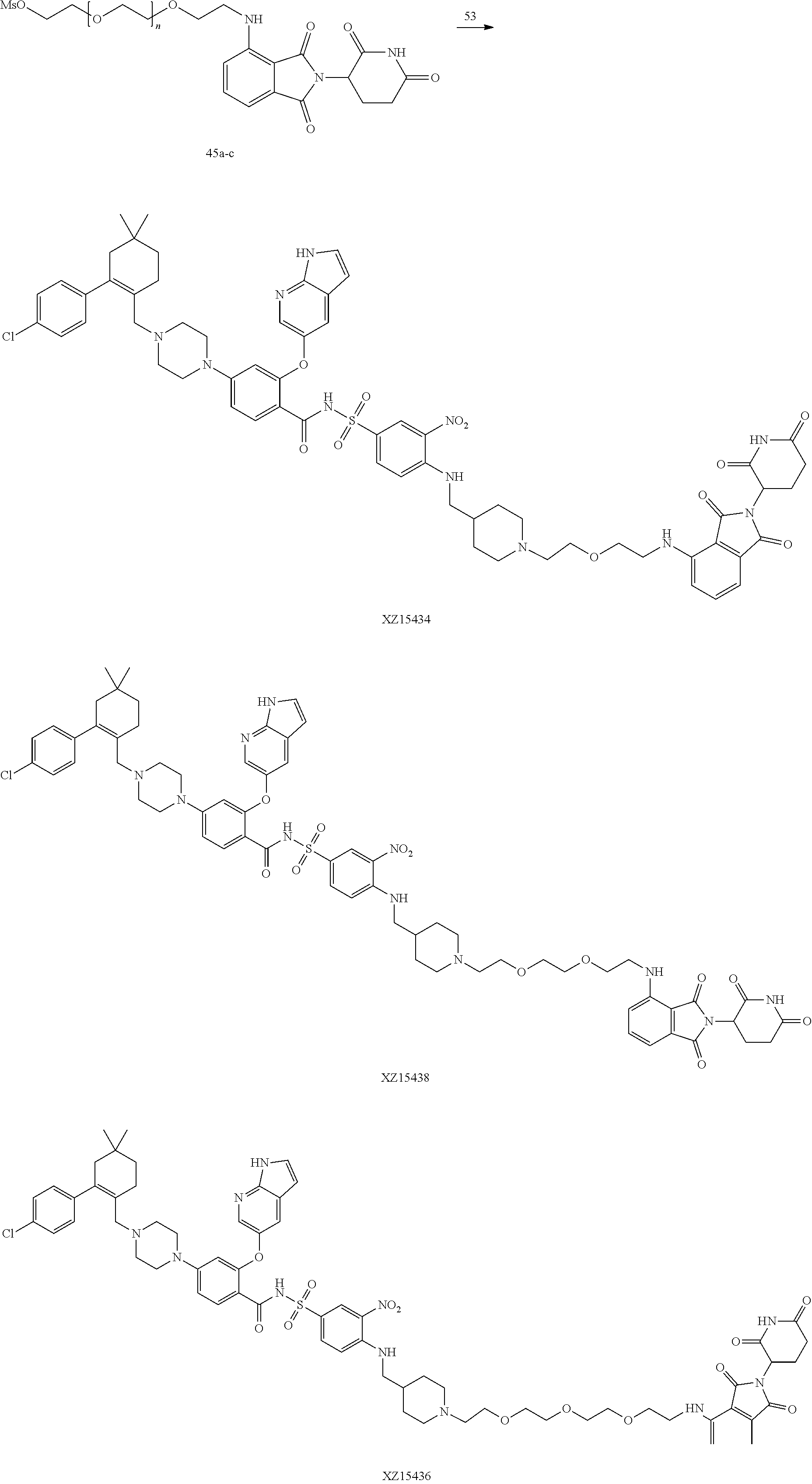

### Example 17: Synthesis of XZ-15434

### Preparation of 2-(1H-pyrrolo[2,3-b]pyridin-5-yloxy)-4-(4-((2-(4-chlorophenyl)-4,4-dimethylcyclohex-1-enyl)methyl)piperazin-1-yl)-N-(4-((1-(2-(2-(2-(2,6-dioxopiperidin-3-0-1,3-dioxoisoindolin-4-ylamino)ethoxy)ethyl)piperidin-4-yl)methylamino)-3-nitrophenylsulfonyl)benzamide (XZ-15434)

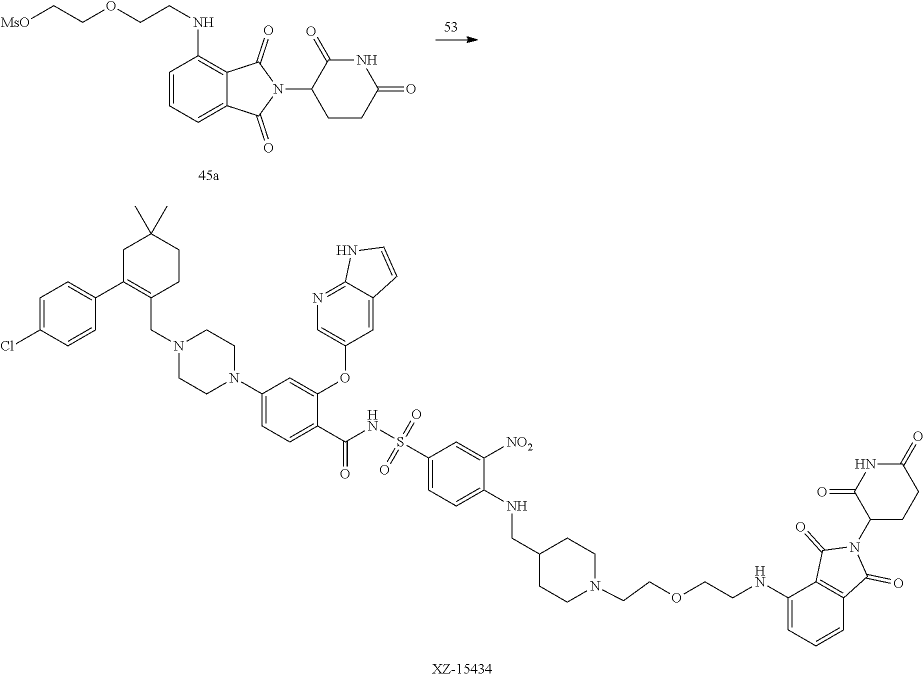 [SMILES: CS(=O)(=O)OCCOCCNc1cccc2c1C(=O)N(C1CCC(=O)NC1=O)C2=O ; CC1(C)CCC(CN2CCN(c3ccc(C(=O)NS(=O)(=O)c4ccc(NCC5CCN(CCOCCNc6cccc7c6C(=O)N(C6CCC(=O)NC6=O)C7=O)CC5)c([N+](=O)[O-])c4)c(Oc4cnc5[nH]ccc5c4)c3)CC2)=C(c2ccc(Cl)cc2)C1]

A mixture of compound 53 (37 mg), compound 45a (14 mg), DIPEA (100 μL), and NaI (3 mg) in 2 mL 1,4-dioxane was heated at 90° C. overnight. The mixture was then poured into water and extracted with EtOAc. The organic phase was washed with water×1, NH₄Cl (aq.)×1, brine×1, dried over Na₂SO₄, filtered and evaporated to dryness. The crude product was purified by column chromatography using DCM and methanol as eluents to afford 10.7 mg compound XZ-15434. Yield 31%. ¹H NMR (400 MHz, CDCl₃ and CD₃OD) δ 8.77 (s, 1H), 8.43 (s, 1H), 8.04 (s, 1H), 7.94-7.83 (m, 2H), 7.59 (s, 1H), 7.50 (dd, J=8.5, 7.2 Hz, 1H), 7.42 (d, J=3.4 Hz, 1H), 7.26-7.17 (m, 2H), 7.09 (d, J=7.0 Hz, 1H), 6.96-6.87 (m, 3H), 6.75 (s, 1H), 6.55 (dd, J=9.1, 2.1 Hz, 1H), 6.45 (d, J=2.8 Hz, 1H), 6.03 (s, 1H), 5.00-4.86 (m, 1H), 3.96-3.88 (m, 2H), 3.75-3.68 (m, 2H), 3.57-3.44 (m, 4H), 3.30-2.58 (m, 15H), 2.24-1.83 (m, 12H), 1.45-1.31 (m, 4H), 0.92 (s, 6H) ppm.

### Example 18: Synthesis of XZ-15438

### Preparation of 2-(1H-pyrrolo[2,3-b]pyridin-5-yloxy)-4-(4-((2-(4-chlorophenyl)-4,4-dimethylcyclohex-1-enyl)methyl)piperazin-1-yl)-N-(4-O-(2-(2-(2-(2-(2,6-dioxopiperidin-3-yl)-1,3-dioxoisoindolin-4-ylamino)ethoxy)ethoxy)ethyl)piperidin-4-yl)methylamino)-3-nitrophenylsulfonyl)benzamide (XZ-15438)

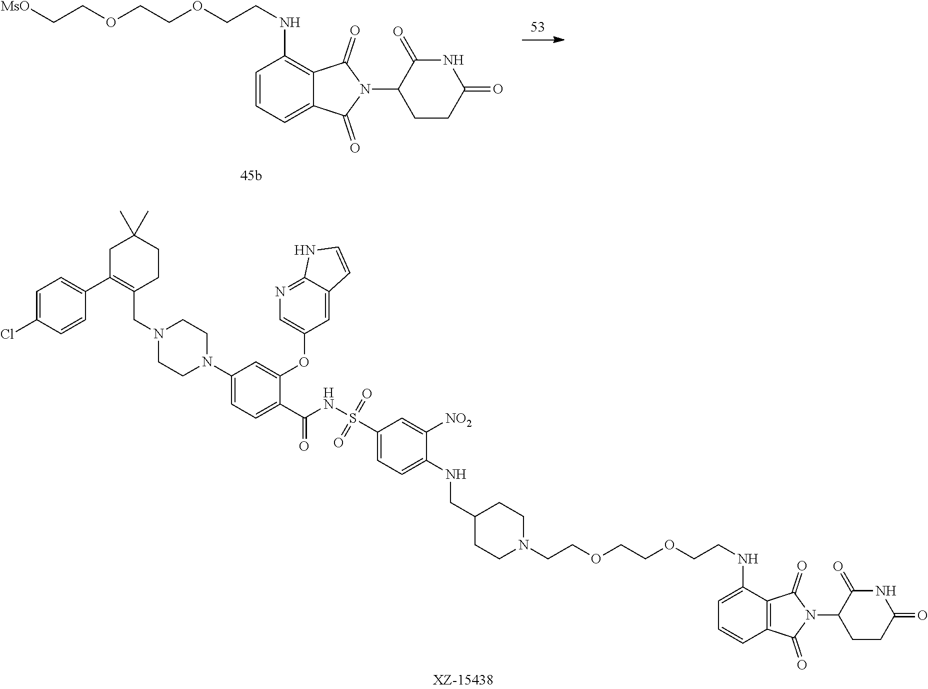 [SMILES: CS(=O)(=O)OCCOCCOCCNc1cccc2c1C(=O)N(C1CCC(=O)NC1=O)C2=O ; CC1(C)CCC(CN2CCN(c3ccc(C(=O)NS(=O)(=O)c4ccc(NCC5CCN(CCOCCOCCNc6cccc7c6C(=O)N(C6CCC(=O)NC6=O)C7=O)CC5)c([N+](=O)[O-])c4)c(Oc4cnc5[nH]ccc5c4)c3)CC2)=C(c2ccc(Cl)cc2)C1]

A mixture of compound 53 (37 mg), compound 45b (15 mg), DIPEA (100 μL), and NaI (3 mg) in 2 mL 1,4-dioxane was heated at 90° C. overnight. The mixture was then poured into water and extracted with EtOAc. The organic phase was washed with water×1, NH₄Cl (aq)×1, brine×1, dried over Na₂SO₄, filtered and evaporated to dryness. The crude product was purified by column chromatography using DCM and methanol as eluents to afford 11.7 mg compound XZ-15438. Yield 31%. ¹H NMR (400 MHz, CDCl₃ and CD₃OD) δ 10.61 (br s, 1H), 10.19 (br s, 1H), 8.81 (s, 1H), 8.39 (s, 1H), 8.04 (s, 1H), 7.87 (d, J=8.9 Hz, 1H), 7.74 (s, 1H), 7.62 (s, 1H), 7.48 (d, J=8.0 Hz, 2H), 7.23 (d, J=8.2 Hz, 2H), 7.06 (d, J=7.0 Hz, 1H), 6.97-6.82 (m, 3H), 6.70-6.43 (m, 4H), 6.01 (s, 1H), 5.15-4.90 (m, 1H), 4.17-3.83 (m, 2H), 3.78-3.58 (m, 8H), 3.48-3.40 (m, 2H), 3.21-2.73 (m, 15H), 2.25-1.94 (m, 12H), 1.45-1.33 (m, 4H), 0.93 (s, 6H) ppm.

### Example 19: Synthesis of XZ-15436

### Preparation of 2-(1H-pyrrolo[2,3-b]pyridin-5-yloxy)-4-(4-((2-(4-chlorophenyl)-4,4-dimethylcyclohex-1-enyl)methyl)piperazin-1-yl)-N-(4-((1-(2-(2-(2-(2-(2-(2,6-dioxopiperidin-3-yl)-1,3-dioxoisoindolin-4-ylamino)ethoxy)ethoxy)ethoxy)ethyl)piperidin-4-yl)methylamino)-3-nitrophenylsulfonyl)benzamide (XZ-15436)

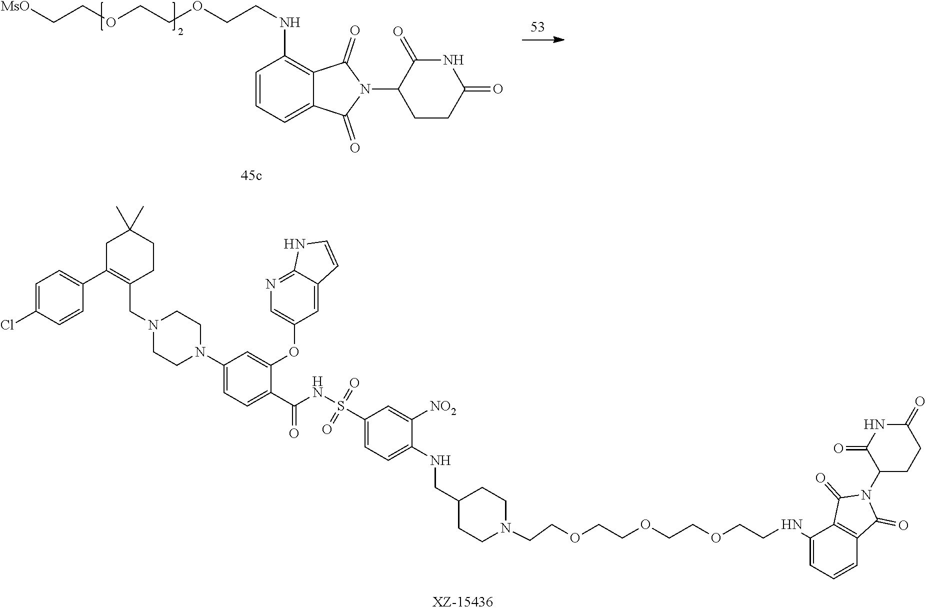

A mixture of compound 53 (37 mg), compound 45c (16 mg), DIPEA (100 μL), and NaI (3 mg) in 2 mL 1,4-dioxane was heated at 90° C. overnight. The mixture was then poured into water and extracted with EtOAc. The organic phase was washed with water×1, NH₄Cl (aq)×1, brine×1, dried over Na₂SO₄, filtered and evaporated to dryness. The crude product was purified by column chromatography using DCM and methanol as eluents to afford 15.0 mg compound XZ-15436. Yield 39%. ¹H NMR (400 MHz, CDCl₃ and CD₃OD) δ 10.59 (br s, 1H), 10.06 (br s, 1H), 8.83 (d, J=2.2 Hz, 1H), 8.43 (t, J=5.4 Hz, 1H), 8.11 (d, J=2.3 Hz, 1H), 7.90 (d, J=9.1 Hz, 2H), 7.63 (d, J=2.4 Hz, 1H), 7.54-7.42 (m, 2H), 7.22 (d, J=8.4 Hz, 2H), 7.07 (d, J=7.1 Hz, 1H), 6.98-6.84 (m, 3H), 6.73 (d, J=9.2 Hz, 1H), 6.61-6.45 (m, 3H), 6.01 (d, J=2.0 Hz, 1H), 5.01-4.93 (m, 1H), 3.94-3.79 (m, 2H), 3.76-3.61 (m, 10H), 3.54-3.40 (m, 4H), 3.27-3.17 (m, 2H), 3.16-2.43 (m, 13H), 2.28-2.09 (m, 7H), 1.98-1.82 (m, 5H), 1.41 (t, J=6.4 Hz, 2H), 1.26-1.23 (m, 2H), 0.93 (s, 6H) ppm.

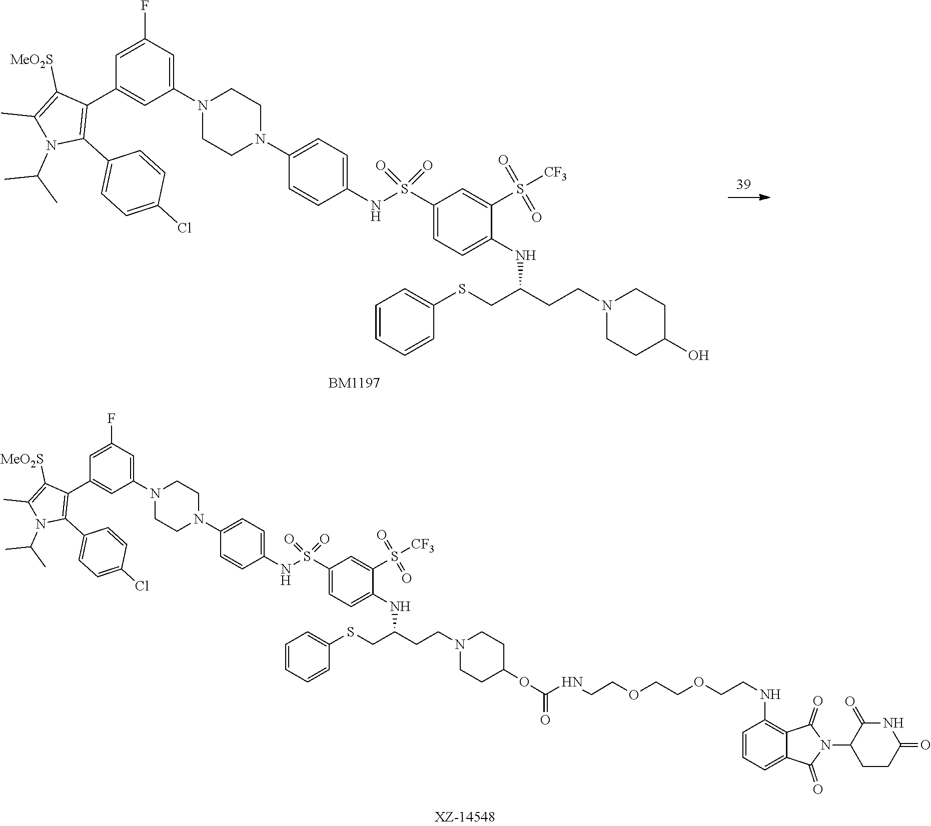 [SMILES: Cc1c(S(C)(=O)=O)c(-c2cc(F)cc(N3CCN(c4ccc(NS(=O)(=O)c5ccc(N[C@H](CCN6CCC(O)CC6)CSc6ccccc6)c(S(=O)(=O)C(F)(F)F)c5)cc4)CC3)c2)c(-c2ccc(Cl)cc2)n1C(C)C ; Cc1c(S(C)(=O)=O)c(-c2cc(F)cc(N3CCN(c4ccc(NS(=O)(=O)c5ccc(N[C@H](CCN6CCC(OC(=O)NCCOCCOCCNc7cccc8c7C(=O)N(C7CCC(=O)NC7=O)C8=O)CC6)CSc6ccccc6)c(S(=O)(=O)C(F)(F)F)c5)cc4)CC3)c2)c(-c2ccc(Cl)cc2)n1C(C)C]

### Example 20: Synthesis of XZ-14548

### Preparation of 1-((R)-3-((4-(N-(4-(4-(3-(2-(4-chlorophenyl)-1-isopropyl-5-methyl-4-(methylsulfonyl)-1H-pyrrol-3-yl)-5-fluorophenyl)piperazin-1-yl)phenyl)sulfamoyl)-2-((trifluoromethyl)sulfonyl)phenyl)amino)-4-(phenylthio)butyl)piperidin-4-yl (2-(2-(2-((2-(2,6-dioxopiperidin-3-yl)-1,3-dioxoisoindolin-4-yl)amino)ethoxy)ethoxy)ethyl)carbamate (XZ-14548)

A mixture of BM1197 (11.5 mg) and CDI (10 mg) in 1.5 mL dichloroethane was heated at 60° C. overnight. A solution of compound 39 (5.5 mg) and TEA (0.3 mL) in 0.5 mL DMSO was then added and the resulted mixture was heated to 70° C. overnight. After cooled to room temperature, the mixture was poured into water and extracted with EtOAc. The organic phase was washed with water×1, brine×1, dried over Na₂SO₄, filtered and evaporated to dryness. The crude product was purified by column chromatography using DCM and methanol as eluents to afford 3.6 mg compound XZ-14548. Yield 22%. ¹H NMR (400 MHz, CDCl₃) δ 8.79 (br s, 1H), 7.99 (s, 1H), 7.65-7.56 (m, 1H), 7.54-7.46 (m, 1H), 7.37-7.27 (m, 4H), 7.26-7.20 (m, 2H), 7.14-7.05 (m, 3H), 7.03-6.94 (m, 3H), 6.91 (d, J=8.4 Hz, 1H), 6.81 (d, J=8.8 Hz, 2H), 6.70 (s, 1H), 6.66-6.50 (m, 2H), 6.43 (d, J=11.9 Hz, 1H), 6.30 (d, J=8.7 Hz, 1H), 5.34-5.26 (m, 1H), 4.98-4.88 (m, 1H), 4.73-4.55 (m, 1H), 4.48-4.34 (m, 1H), 3.96-3.81 (m, 1H), 3.75-3.37 (m, 12H), 3.22-3.03 (m, 10H), 2.96-2.61 (m, 10H), 2.57-2.43 (m, 1H), 2.40-1.63 (m, 11H), 1.45 (d, J=7.1 Hz, 6H) ppm.

### Example 21: Evaluation of Compounds of Formula (I) and Formula (II) for their Ability to Selectively Kill Senescent Cells

Normal WI38 (NSC) and IR-induced senescent WI38 cells (IR-SC) were incubated with vehicle or increasing concentrations of compounds of Formula (I) or Formula (II) for 72 hours. The cells were digested with 0.25% trypsin and 1 mM EDTA, and harvested in PBS containing 2% FBS. After incubation with propidium iodide (PI, 100 ng/ml) in PBS at room temperature for 1 minute, cells were centrifuged at 1,200 rpm for 6 minutes to remove PI and then resuspended in PBS containing 2% FBS for analysis using a flow cytometer. Viable cells (PI− cells) were analyzed by flow cytometry at a constant flow rate to count the number of cells and calculated as a percentage of control cells treated with vehicle. Table 1 depicts the EC50 values of the compounds of Formula (I) and compounds of Formula (II) against normal WI38 and IR-induced senescent WI38.

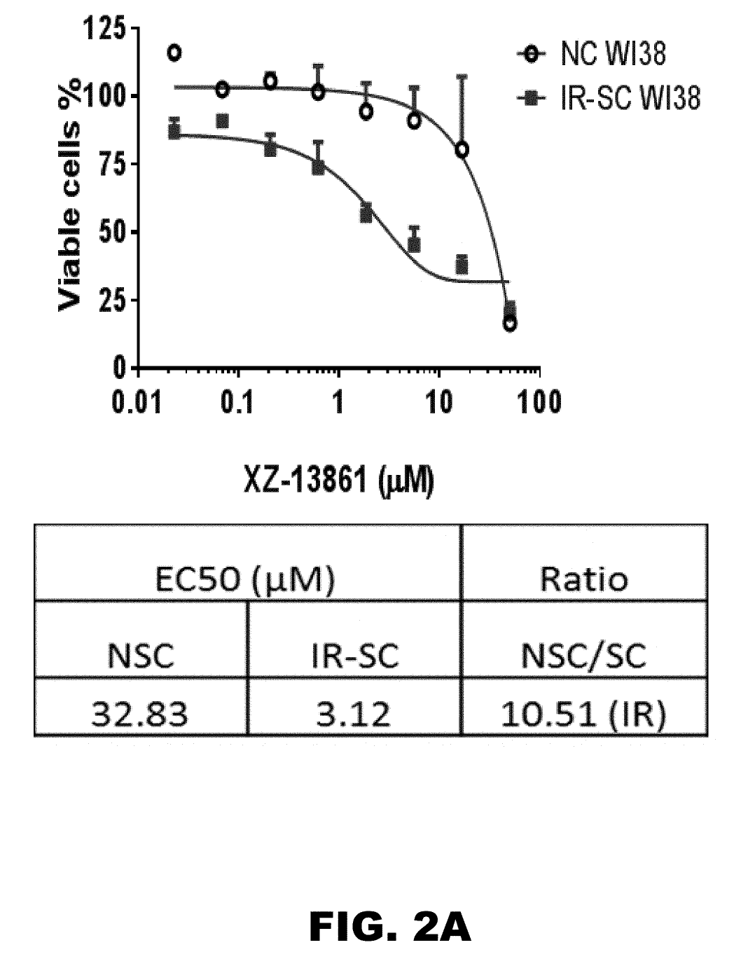

Both compound 11 (XZ-13861) (FIG. 2A) and XZ-12906 (FIG. 2B) selectively inhibit IR-SC WI38 cells but not normal WI 38 cells in a dose dependent manner.

### Example 22: Evaluation of Compounds of Formula (I) and Formula (II) for their Ability to Kill Cancer Cells

RS4; 11 and NCI-H146 cancer cells were incubated with vehicle or increasing concentrations of compounds of Formula (I) and Formula (II). At 72 hours post-treatment cell viability was measured by MTS and EC50 was calculated as a percentage of control cells treated with vehicle. Table 1 depicts the EC50 values of compounds of Formula (I) and Formula (II) against RS4; 11 and NCI-H146.

TABLE 1
EC50 values of compounds of Formula (I) and
Formula (II) against RS4; 11 and NCI-H146.

| | WI38 EC50 | | | EC50 | |
|---|---|---|---|---|---|
| | (μM) | | Ratio | (nM) | |
| Compound | NSC | IR-SC | NSC/SC | RS4 | NCI-H146 |
| ABT-263 | 12.6 | 0.61 | 20.6 | 16.00 | 27.47 |
| XZ-14439 | >20 | 0.87 | >20.88 | 84.43 | 69.55 |
| PZ-15227 | >10 | 0.108 | >92 | 113.62 | 68.86 |
| XZ-15421 | >10 | <0.1562 | >64.02 | 72.92 | |
| XZ-14510 | >10 | 0.177 | >56 | 219.89 | 111.98 |
| XZ-14509 | >10 | 0.226 | >44.22 | 90.00 | 64.95 |
| XZ-14515 | >10 | 0.158 | >63.27 | 173.16 | 79.17 |
| XZ-14516 | >10 | 0.092 | >109 | 1088.63 | 517.16 |
| XZ-14540 | >10 | 1.13 | 8.86 | 82.43 | 76.25 |
| XZ-14437 | >20 | 4.55 | >4.4 | 460 | >250 |
| XZ-14529 | >10 | >10 | — | 822.56 | >2000 |
| XZ-15416 | >10 | <0.1562 | >64.02 | 30.71 | >10 |
| XZ-15405 | >10 | 0.152 | >65.97 | 24.78 | |
| XZ-15418 | >10 | 0.172 | >58.06 | 12.33 | |
| ABT-199 | >10 | >10 | — | | |
| XZ-14522 | >10 | >10 | — | 46.98 | 1103.7 |
| XZ-14523 | >10 | >10 | — | 160.86 | 1255.91 |
| XZ-14528 | >10 | >10 | — | 396.62 | >2000 |
| XZ-13906 | 100 | 2.4 | 41.67 | >2000 | >2000 |
| XZ-14455 | 5.0 | 1.47 | 3.40 | >2000 | >2000 |
| XZ-14424 | 1.6 | 1.4 | 1.2 | | |
| XZ-13861 | 32.8 | 3.12 | 10.51 | | |

### Example 23: Protein Degradation Assays in Senescent Cells

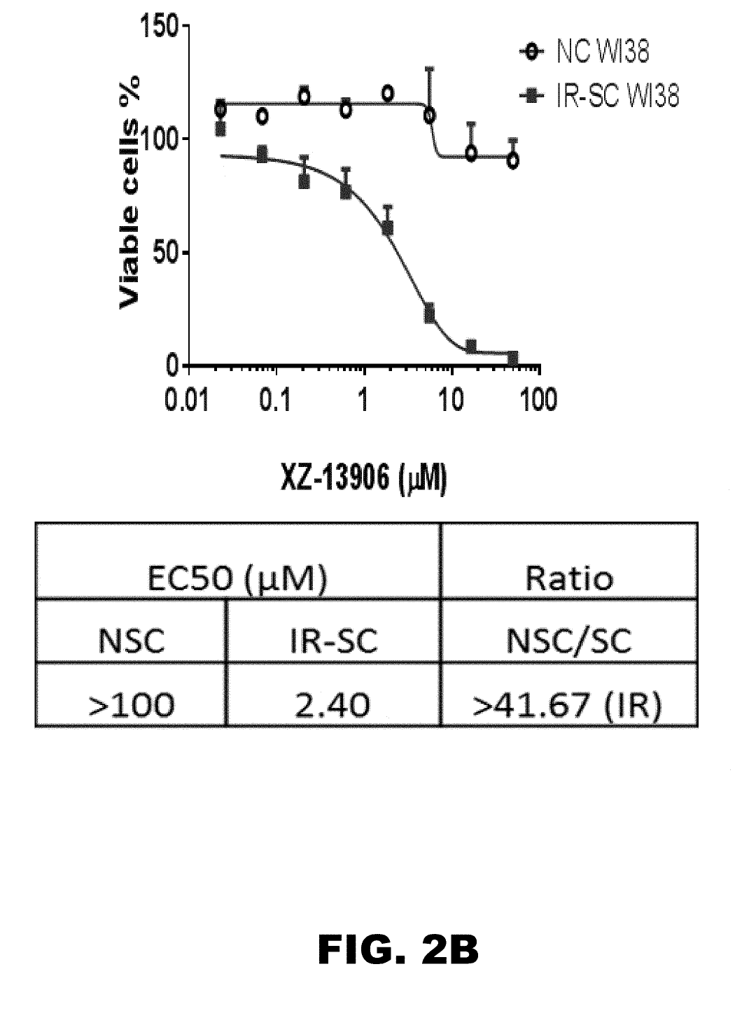

IR-SC WI38 cells were incubated with vehicle or increasing concentrations of XZ-14439 for 18 hours at increasing contractions (FIG. 3A) and at a fixed concentration of XZ-14439 for increasing times (FIG. 3B). The cells were digested with 0.25% trypsin and 1 mM EDTA, and harvested in RIPA lysis buffer with 1% Phosphatase Inhibitor Cocktail 3 and 1% Protease Inhibitor Cocktail. An equal amount of protein (15-30 μg/lane) from each cell extract was resolved on a 12% SDS-PAGE gel. Proteins were blotted onto a NOVEX PVDF membrane by electrophoresis. The membranes were blocked with TBS-T blocking buffer (5% nonfat milk in 25 mM Tris-HCL, pH 7.4; 3 mM KCl; 1n40 mM NaCl; and 0.05% Tween) and probed with primary antibodies (at a predetermined optimal concentration) overnight at 4° C. or for 1 hour at room temperature. After extensive washing with TBS-T, the membranes were incubated with an appropriate peroxidase-conjugated secondary antibody for 1 hour at room temperature. After three washes with TBS-T, the proteins of interest were detected with ECL Western Blotting Detection Reagents and recorded with autoradiography (Pierce Biotech, Rockford, Ill., USA). The primary antibody Bcl-xl (#2762), Bcl-2 antibody (#2872S) and β-actin (13E5, #4970) were purchased from Cell Signaling.

XZ-14439 depletes Bcl-xL in IR-SC WI38 cells in both a dose dependent (FIG. 3A) and time dependent (FIG. 3B) manner.

### Example 24: Protein Degradation Assays in Cancer Cells

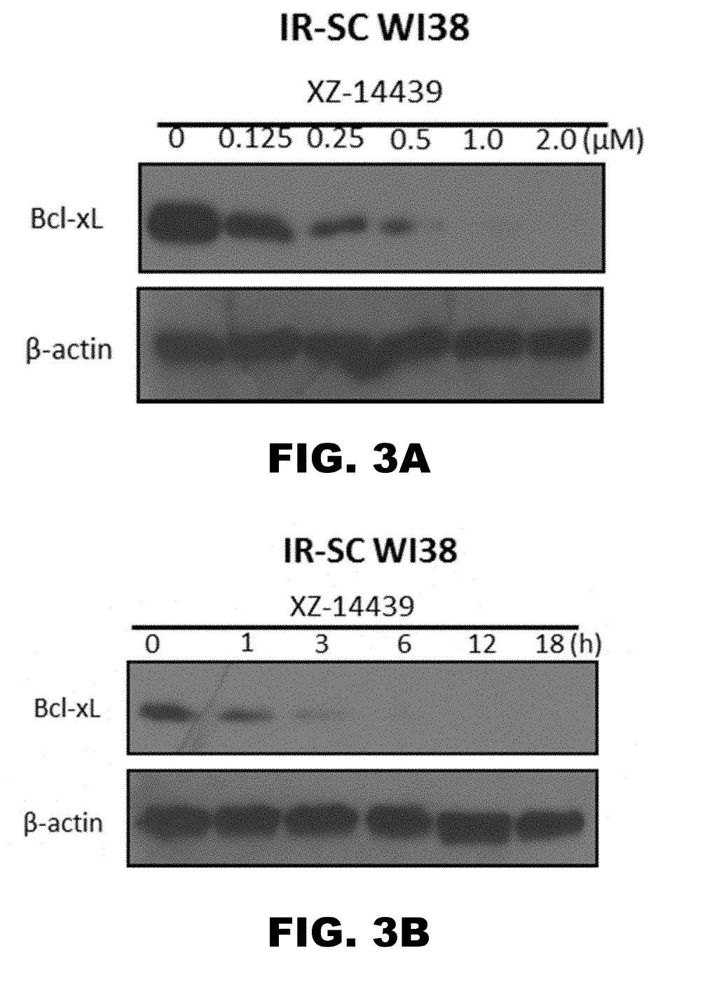

IR-SC WI38 (FIG. 4A) and RS4; 11 (FIG. 4B) cells were incubated with vehicle or increasing concentrations of compounds of Formula (I) or Formula (II) for 6 or 16 hours, at 1 μM (FIG. 4A) and 100 nm (FIG. 4B), respectively. The cells were harvested in RIPA lysis buffer with 1% Phosphatase Inhibitor Cocktail 3 and 1% Protease Inhibitor Cocktail. An equal amount of protein (15-30 μg/lane) from each cell extract was resolved on a 12% SDS-PAGE gel. Proteins were blotted onto a NOVEX PVDF membrane by electrophoresis. The membranes were blocked with TBS-T blocking buffer (5% nonfat milk in 25 mM Tris-HCL, pH 7.4; 3 mM KCl; 140 mM NaCl; and 0.05% Tween) and probed with primary antibodies (at a predetermined optimal concentration) overnight at 4° C. or for 1 hour at room temperature. After extensive washing with TBS-T, the membranes were incubated with an appropriate peroxidase-conjugated secondary antibody (Jackson ImmunoResearch Europe, Suffolk, UK) for 1 hour at room temperature. After three washes with TBS-T, the proteins of interest were detected with ECL Western Blotting Detection Reagents and recorded with autoradiography. The primary antibody Bcl-xl (#2762), Bcl-2 antibody (#2872S), Bcl-w (#2724S), Mcl-1 (#5453s) and β-actin (13E5, #4970) were purchased from Cell Signaling.

XZ-15416, XZ-15405, XZ-15418, XZ-15421, and PZ-15227 deplete Bcl-xL in IR-SC WI38 and RS4; 11 cells at 1 μM (FIG. 4A) and 200 nM (FIG. 4B).

### Example 25: Efficacy of Senolytic Agents for CIPN

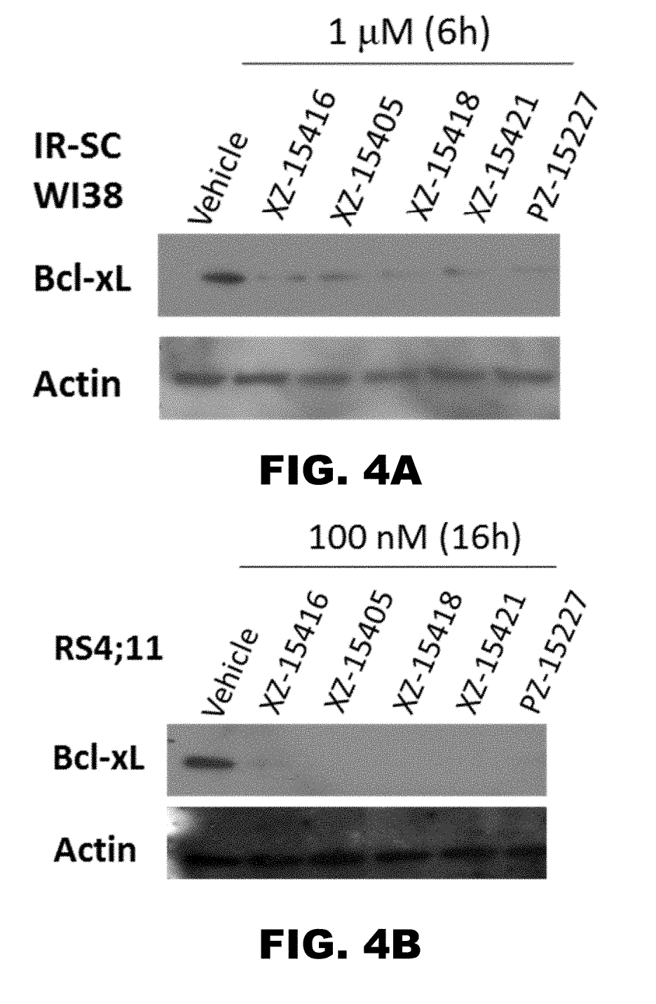

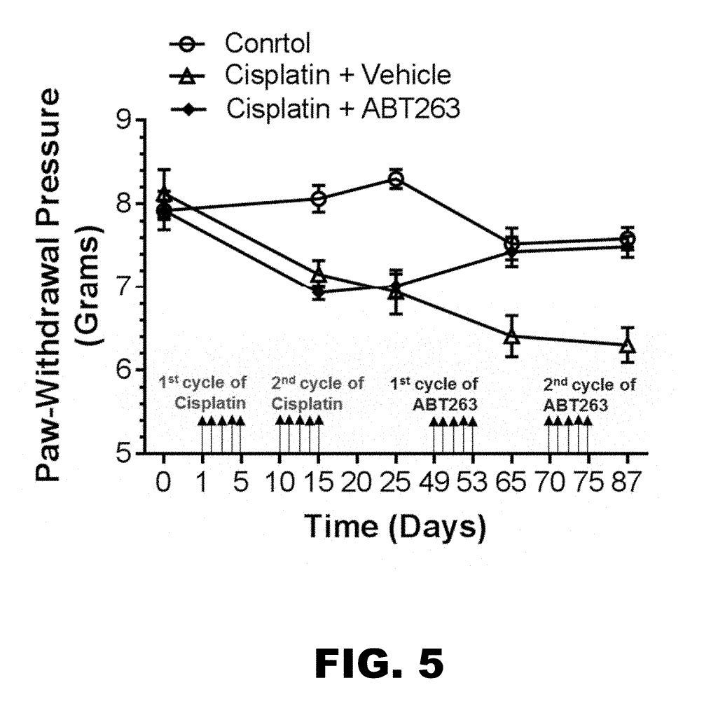

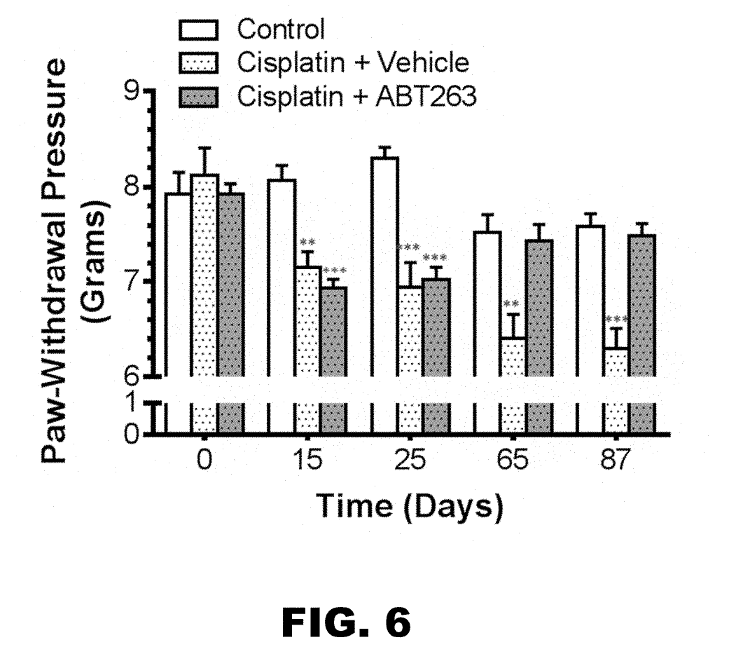

To determine the effect of senescent cells on the induction of CIPN, Cisplatin-treated mice were administered with vehicle or ABT263 at 50 mg/kg/day for 5 days per cycle for 2 cycles with an interval of 17 days between the cycles by i.p. injection at the indicated times after Cisplatin treatment. The induction of CIPN was measured by analyzing the mechanical sensitivity using the Von Frey assay (FIG. 5 and FIG. 6) and by analyzing the sensitivity to thermal allodynia using the Hot Plate Analgesia Meter (FIG. 7) the day before Cisplatin treatment and on various day after Cisplatin treatment. As can be seen in FIG. 5-7, the clearance of senescent cells with ABT263 reversed Cisplatin-induced peripheral neuropathy (CIPN) in C57BL/6 mice.

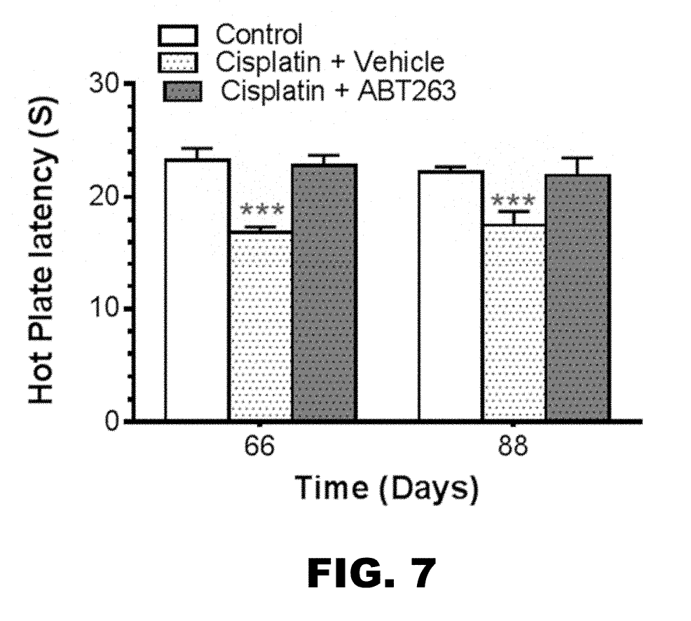

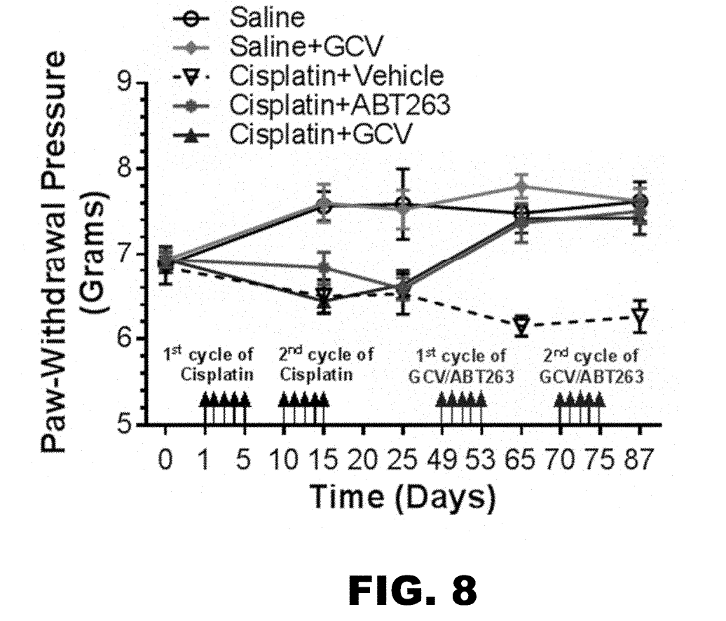

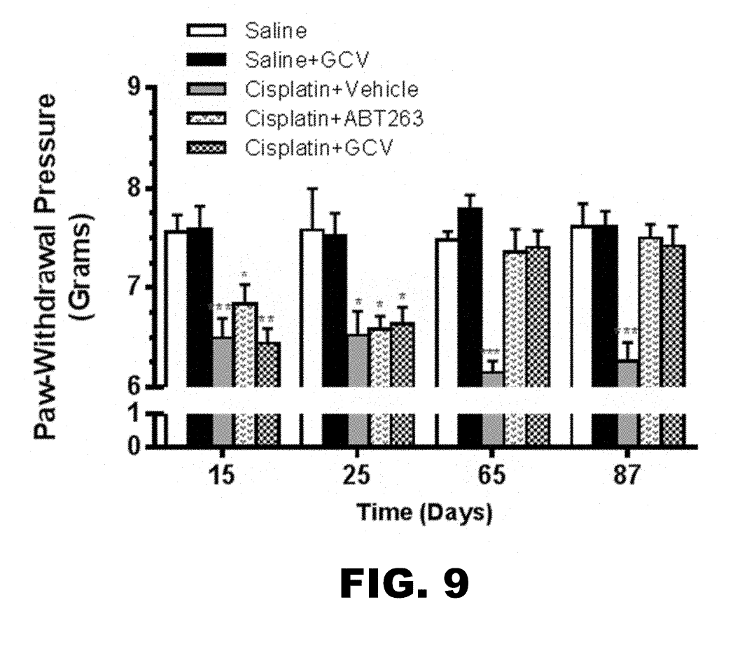

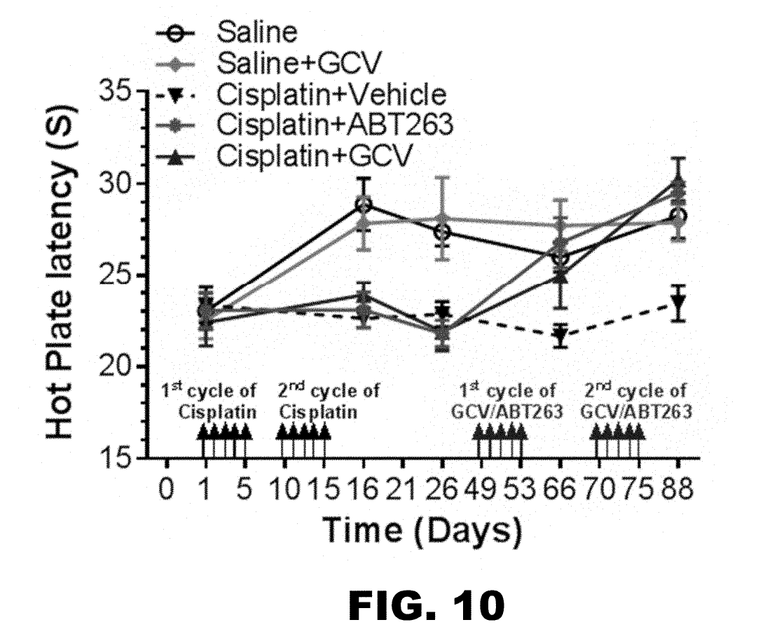

To determine the effect of senescent cells on the induction of CIPN, cisplatin-treated mice were administered with vehicle, ganciclovir (GCV) at 25 mg/kg/day for 5 days per cycle for 2 cycles with an interval of 17 days between the cycles, or ABT263 at 50 mg/kg/day for 5 days per cycle for 2 cycles with an interval of 17 days between the cycles by i.p. injection at the indicated times after cisplatin treatment. P16-3MR transgenic mouse model is used to eliminate cells undergoing cellular senescence. In this model, the senescence-sensitive promoter from the cyclin-dependent kinase inhibitor 2A (Cdkn2a) gene, also known as p16INK4a, drives expression of 3MR, a fusion protein that is composed of luciferase and red fluorescent protein (RFP) reporters and herpes simplex virus-1 thymidine kinase, which converts ganciclovir (GCV) into an apoptosis inducer. The induction of CIPN was measured by analyzing the mechanical sensitivity using the Von Frey assay (FIG. 8 and FIG. 9) and by analyzing the sensitivity to thermal allodynia using the Hot Plate Analgesia Meter (FIG. 10 and FIG. 11) the day before Cisplatin treatment and on various day after Cisplatin treatment. As can be seen in FIG. 8-11, both genetic and pharmacological clearance of senescent cells with GCV and ABT263, respectively, reversed cisplatin-induced peripheral neuropathy (CIPN) in p16-3MR transgenic mice.

### Example 25: Efficacy of Formula (I) or Formula (II) for Osetoporosis

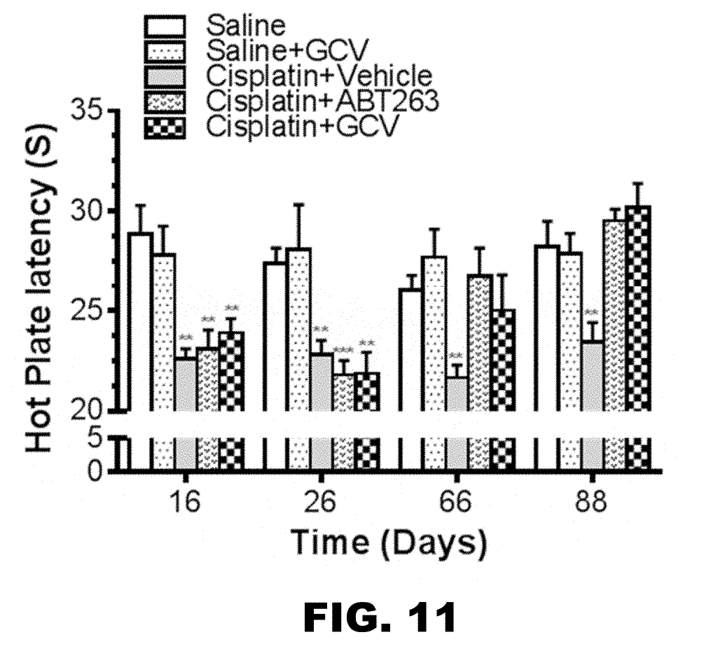

ABT-263 or its derivative Bcl-PROTAC (or vehicles) were administered by daily i.p. injections for 5 days to 24-month-old female mice. Osteocyte-enriched femoral bone shafts from mice treated with senolytics had decreased marker of cell senescence including protein levels of γ-H2AX, p16, and GATA4 (FIG. 12A). Furthermore, short-term administration of the senolytics decreased the mRNA for RANKL and MMP13, but increased the mRNA for OPG (FIG. 12B). Interestingly, Bcl-PROTAC also increased the mRNA for Wnt10b (FIG. 12B), a canonical Wnt ligand that induces bone formation in mice. We have shown that γ-H2AX, p16, GATA4, RANKL and MMP-13 increase, while OPG and Wnt10b decrease with age in bone of female and male mice (Piemontese et al., 2017 JCI Insight).

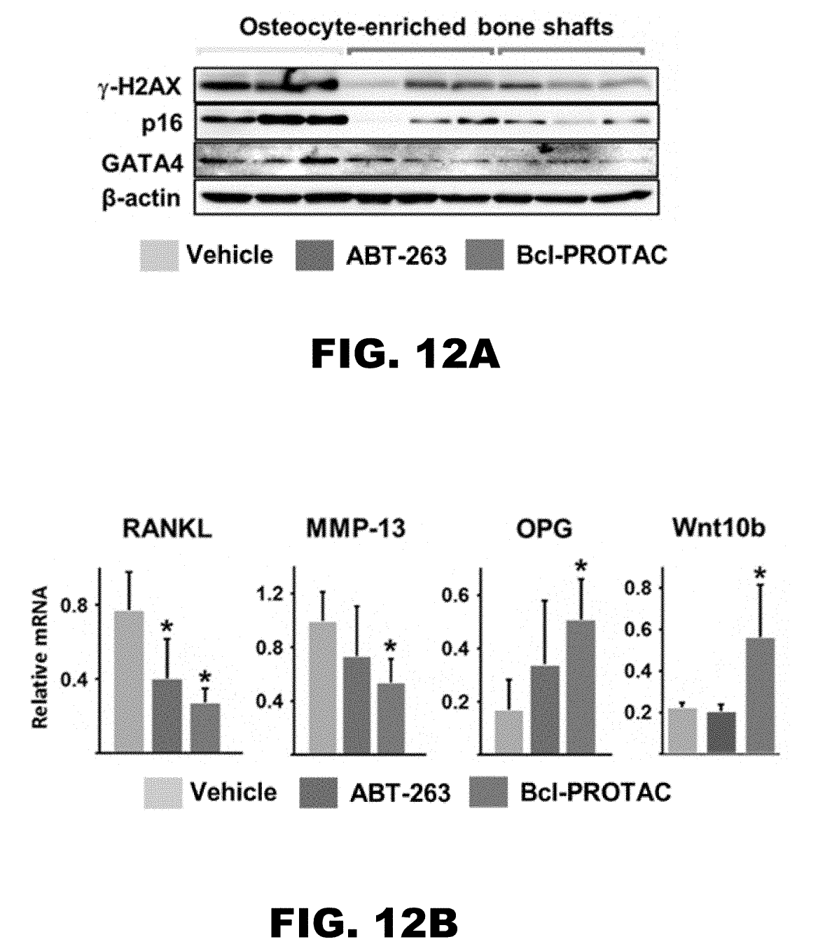

We also isolated bone marrow stromal cells from mice treated with vehicle or the senolytics and cultured them for 5 days. Similar to the findings from the osteocyte-enriched bone shafts, bone marrow-derived stromal cells from mice treated with senolytics had decreased mRNA levels of RANKL and MMP-13, as well as IL-1a and SDF-1 (FIG. 13). Additionally, these ex-vivo cultures had decreased expression of p21. Bcl-PROTAC was more potent than ABT-263 in these effects (FIG. 13).

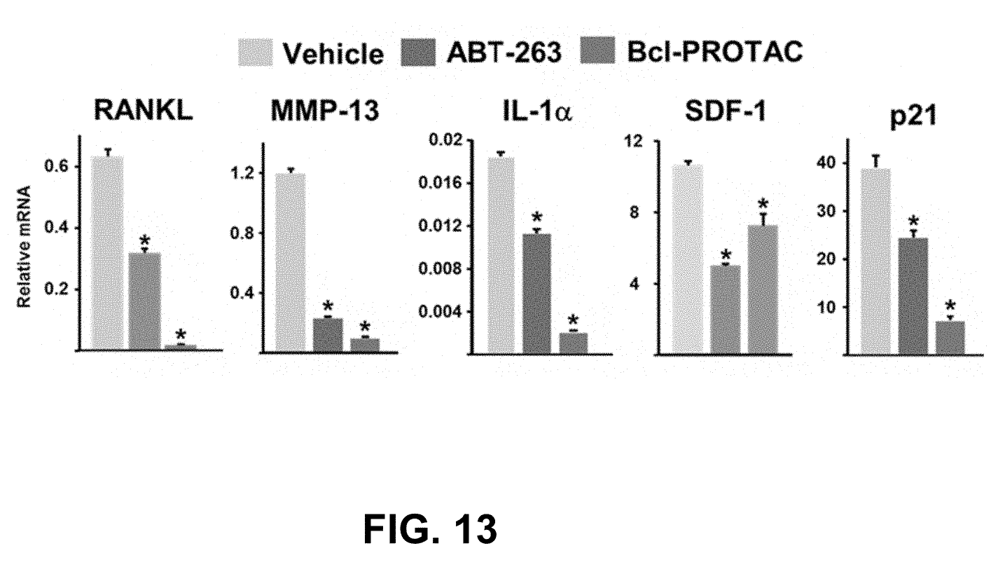

In agreement with the decreased cell senescence, cultured bone marrow stromal cells from mice treated with senolytics exhibited increased osteoblastogenic potential as evidence by an increase in Alizarin Red staining after 21 days of culture in the presence of osteogenic medium (FIGS. 14A and 14B). Conversely, bone marrow cells from the mice treated with senolytics cultured in the presence of adipogenic medium had decreased adipogenic potential (FIG. 14C).

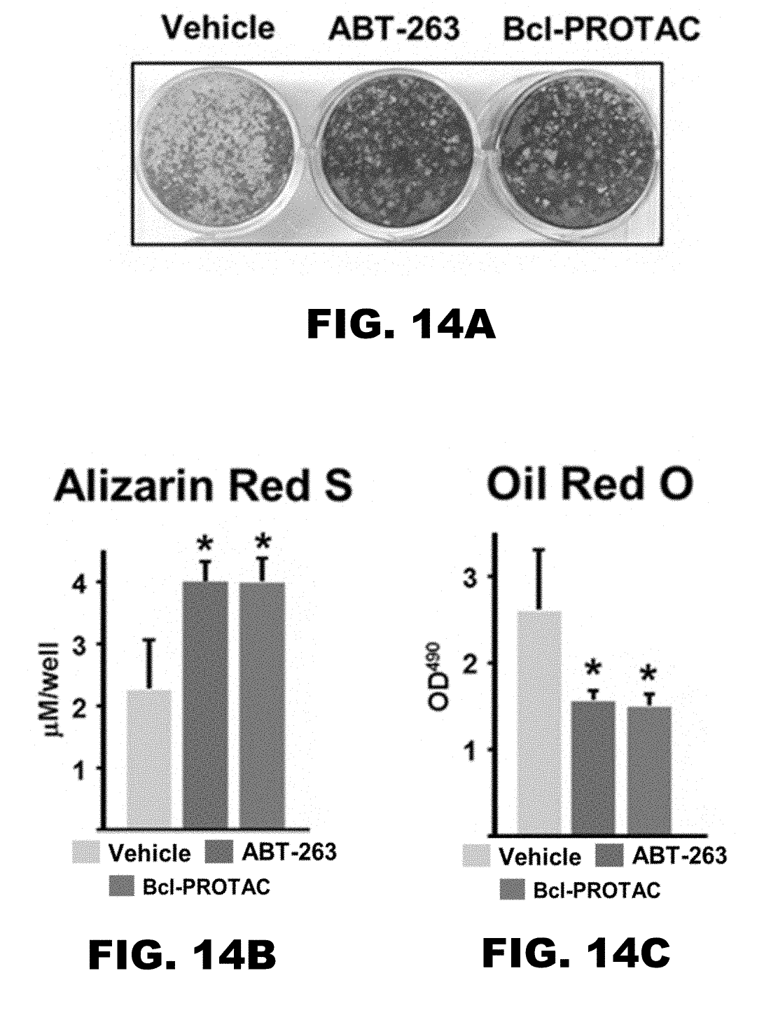

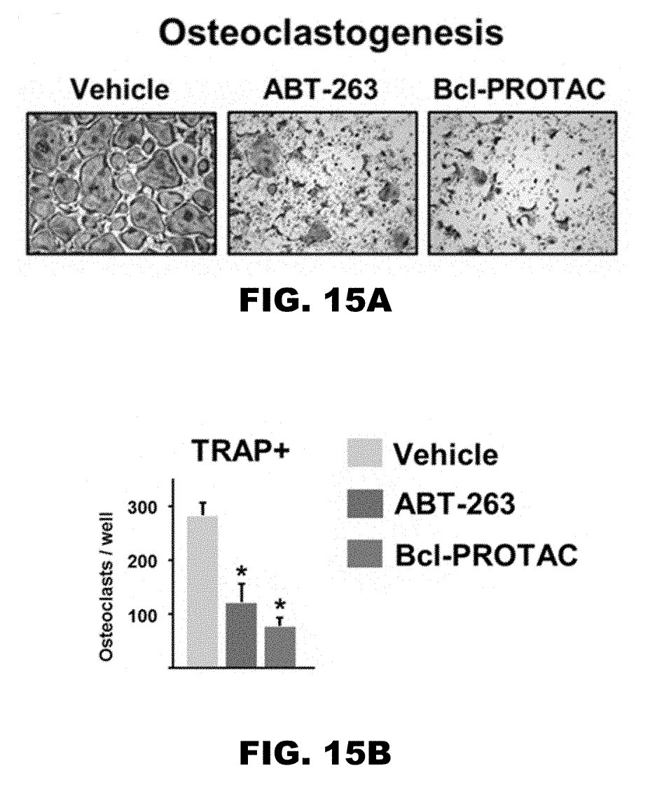

Furthermore, macrophages isolated from the bone marrow of mice treated with senolytics had decreased osteoclastogenic potential in cultures maintained in the presence of RANKL and M-CSF for 5 days (FIGS. 15A and 15B). In summary, short-term administration of ABT-263 or Bcl-PROTAC to aged mice decreases senescent osteoprogenitors and osteocytes, as well as the SASP and osteoclastogenesis.

### Methods of Example 25

Western Blotting.

Osteocyte-enriched femoral cortical bone was prepared by removing the ends, flushing the bone marrow by centrifugation, and removal of surface cells by scraping with a scalpel. Bone fragments were immediately frozen in liquid nitrogen and pulverized. Proteins were extracted with a buffer containing 20 mM Tris-HCL, 150 mM NaCl, 1% Triton X-100, protease inhibitor mixture, and phosphatase inhibitor cocktail (Sigma-Aldrich) on ice for 30 min and then keep on −80° C. for overnight. The protein concentration of the bone extract was determined using the DC Protein Assay Kit (Bio-Rad). The extracted protein (20-30 mg per sample) was subjected to 15% SDS-PAGE gels and transferred electrophoretically onto PVDF membranes. The membranes were blocked in 5% fat-free milk/Tris-buffered saline for 120 min and incubate with each primary antibody followed by secondary antibodies conjugated with horseradish peroxidase. Mouse monoclonal antibodies against g-H2AX (Millipore #05-636, 1:5000), mouse monoclonal antibody for p16 (Santa Cruz Biotechnology, sc-1661, 1:2000) and goat polyclonal antibody for GATA4 (Santa Cruz Biotechnology, sc-1237, 1:500) were used to detect their corresponding protein levels. Blots were stripped and reprobed with anti-β-actin antibody (Santa Cruz Biotechnology, sc-81178, 1:2000). Bound antibodies were detected with ECL reagents (Millipore), and imaged and quantified with a VersaDoc™ imaging system (Bio-Rad).

Quantitative PCR.

Total RNA was extracted with Ultraspec (Biotecx Laboratories) and reverse-transcribed using the High-Capacity cDNA Archive Kit (Applied Biosystems) according to the manufacturer's instructions. TaqMan quantitative real-time PCR was performed using the following primers from Applied Biosystems: Rankl (Mm00441908_m1); Mmp-13 (Mm00439491_m1); OPG (Mm00435452_m1); Wnt10b (Mm00442104_m1); IL-1α (Mm99999060_m1); Sdf-1 (Mm00445553_m1); p21 (Mm00432448_m1). Target gene expression was calculated by normalizing to the housekeeping gene ribosomal protein S2 (Mm00475528_m1) using the ΔCt method (Livak and Schmittgen, 2001).

Osteoclast Differentiation.

Bone marrow cells were obtained by flushing the tibiae and femurs from the 24 month-old female mice with α-MEM medium (Invitrogen). After the red blood cells were removed with ACK buffer (0.01 mM EDTA, 0.011 M KHCO3, and 0.155 M NH4Cl, pH 7.3), we suspended the cells in α-MEM complete media containing 10% (v/v) fetal bovine serum, 100 U ml-1 penicillin, and 100 μg ml-1 streptomycin and incubated the cells for 24 h in the presence of 10 ng ml-1 M-CSF. We collected the non-adherent cells and cultured with 30 ng ml-1 M-CSF for 3 days to generate bone marrow-derived macrophages. To generate osteoclasts we cultured bone marrow-derived macrophages (50000 cells cm-2) with 30 ng ml-1 M-CSF and 30 ng ml-1 RANKL (R&D Systems) for 2 and 4 days, respectively, in α-MEM complete media. We fixed the osteoclast cultures with 10% neutral buffered formalin for 15 min and stained for tartrate-resistant acid phosphatase (TRAP) using the Leukocyte Acid Phosphatase Assay Kit, following the manufacturer's instructions (Sigma-Aldrich). For all assays we plated cells in triplicate.

Osteoblast Differentiation.

Total bone marrow cells pooled from 3-5 mice from each group were cultured with 20% FBS, 1% PSG and 50 mg/ml of ascorbic acid in 10-cm culture dishes for 5 days. Half of the medium was replaced every 3 days. Bone marrow-derived osteoprogenitor cells were re-plated into 12-well plates and cultured with 50 mg/ml of ascorbic acid and 10 mM b-glycerophosphate for 21 days. Mineralized matrix was stained with 40 mM Alizarin Red solution.

Adipocyte Differentiation.

Bone marrow-derived stromal cells were cultured to 70% confluence, and the media supplemented with rosiglitazone (5 nM/m1) or with 3.3% BSA in PBS as vehicle control. Medium containing ligand was changed every 2 d for 11 d. Cells were fixed in 10% formalin in PBS, rinsed, and stained for 30 min with 0.15% Oil Red 0 (Sigma) in a 55:45 mix of isopropanol and water. Cells were counterstained with 0.5% methyl green (Fisher Scientific, Fairlawn, N.J.) in 0.1 M sodium acetate, pH4. Oil Red 0 staining was quantified after extraction of the dye with 1 ml isopropanol and absorbance determination at 490 nm.

### REFERENCES

Aguilar, A., et al., (2013) A potent and highly efficacious Bcl-2/Bcl-xL inhibitor, J Med Chem 56: 3048-3067.
Bai. L., et al. (2014) BM-1197: a novel and specific Bcl-2/Bcl-xL inhibitor inducing complete and long-lasting tumor regression in vivo, PLOS One 9:e99404.
Baker, D. J., et al., (2011) Clearance of p16Ink4a-positive senescent cells delays ageing-associated disorders, Nature 479:232-236.
Baker, D. J., et al., (2016) Naturally occurring p16(Ink4a)-positive cells shorten healthy lifespan, Nature 530:184-189.
Bajwa, N., et al., (2012) Inhibitors of the anti-apoptotic Bcl-2 proteins: a patent review, Expert Opin. Ther. Patents 22:37-55.
Bruncko, M., et al., (2007) Studies leading to potent, dual inhibitors of Bcl-2 and Bcl-xL, J Med Chem 50:641-662.
Bruncko, M., et al., (2015) Structure-guided design of a series of MCL-1 inhibitors with high affinity and selectivity, J Med Chem 58:2180-2194.
Campisi, J., (2005) Senescent cells, tumor suppression, and organismal aging: good citizens, bad neighbors, Cell 120:513-522.
Campisi, J., (2011) Cellular senescence: putting the paradoxes in perspective, Curr. Opin. Genet. Dev. 21:107-112.
Chang, J., et al., (2016) Clearance of senescent cells by ABT263 rejuvenates aged hematopoietic stem cells in mice, Nat. Med. 22:78-83.
Chen, J., et al., (2012) Structure-based discovery of BM-957 as a potent small-molecule inhibitor of Bcl-2 and Bcl-xL capable of achieving complete tumor regression, J Med Chem 55:8502-8514.
Delbridge, A. R., et al., (2016) Thirty years of BCL-2: translating cell death discoveries into novel cancer therapies, Nat. Rev. Cancer 16:99-109.
Jing, L., et al., (2015) Hijacking the E3 Ubiquitin Ligase Cereblon to Efficiently Target BRD4, Chem. Biol. 22:755-763, 2015
Rodier, F. and Campisi, J., (2011) Four faces of cellular senescence, J. Cell Biol. 192:547-556.
Park. C. M., et al., (2008) Discovery of an Orally Bioavailable Small Molecule Inhibitor of Prosurvival B-Cell Lymphoma 2 Proteins, J Med Chem 51:6902-6915.
Pelz, N. F., et al., (2016) Discovery of 2-Indole-acylsulfonamide Myeloid Cell Leukemia 1 (Mcl-1) Inhibitors Using Fragment-Based Methods, J Med Chem 59:2054-2066.
Sleebs, B. E., et al., Quinazoline Sulfonamides as Dual Binders of the Proteins B-Cell Lymphoma 2 and B-Cell Lymphoma Extra Long with Potent Proapoptotic Cell-Based Activity (2011) J Med Chem 54:1914-1916.
Sleebs, B. E., et al., (2013) Discovery of Potent and Selective Benzothiazole Hydrazone Inhibitors of Bcl-X_{L}, J Med Chem 56:5514-5540.
Tanaka, Y., et al., (2013) Discovery of potent Mcl-1/Bcl-xL dual inhibitors by using a hybridization strategy based on structural analysis of target proteins, J Med Chem 56:9635-9645.
Tao, Z. F., et al., (2014) Discovery of a Potent and Selective BCL-XL Inhibitor with in Vivo Activity, ACS Med Chem Lett 5:1088-1093.
Zhou, H., et al., (2012) Structure-based design of potent Bcl-2/Bcl-xL inhibitors with strong in vivo antitumor activity, J Med Chem 55:6149-6161.
Lessene, G., et al., (2008) BCL-2 family antagonists for cancer therapy, Nat. Rev. Drug Discov. 7:989-1000.
Vogler, M., et al., (2009) Bcl-2 inhibitors: small molecules with a big impact on cancer therapy, Cell Death Differ. 16:360-367
Vogler, M., (2014) Targeting BCL2-Proteins for the Treatment of Solid Tumours, Adv. Med. 1-14.
Zhu, Y., et al., (2015) The Achilles' heel of senescent cells: from transcriptome to senolytic drugs, Aging Cell 14:644-658.

What is claimed is:

1. A method of treating chemotherapy induced peripheral neuropathy in a subject in need thereof, the method comprising administering a therapeutically-effective amount of at least one senolytic agent.

2. The method of claim 1, wherein the at least one senolytic agent selectively kills senescent cells over non-senescent cells.

3. The method of claim 1, wherein the senolytic agent is administered in at least two treatment cycles.

4. The method of claim 3, wherein the treatment cycle independently comprises a treatment course of from 1 day to 3 months followed by a non-treatment intervals of at least 2 weeks.

5. The method of claim 1, wherein the senolytic agent is a specific inhibitor of Bcl-xL, MDM2, or Akt.

6. The method of claim 1, wherein the senolytic agent is a means for inhibiting Bcl-xL or Bcl-2.

7. The method of claim 1, wherein the senolytic agent is ABT263.

8. The method of claim 1, wherein the senolytic agent is administered locally at or near the site of the disease.

9. The method of claim 1, wherein the step of administering is intravenous, intraperitoneal, subcutaneous, intramuscular, topical, transdermal or oral.

10. The method of claim 1, wherein the senolytic agent is a compound comprising Formula (II): 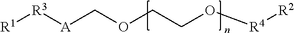
wherein
R¹ is selected from the group consisting of: 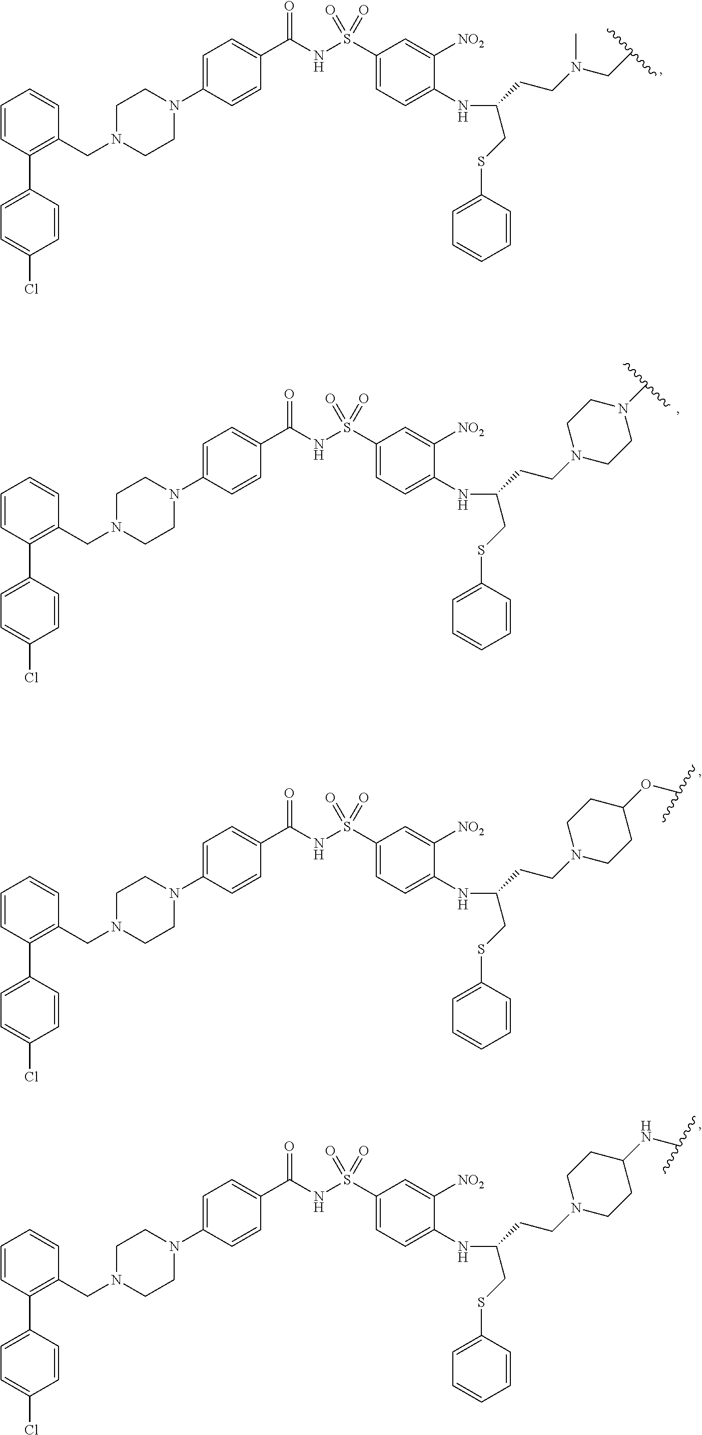 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1] 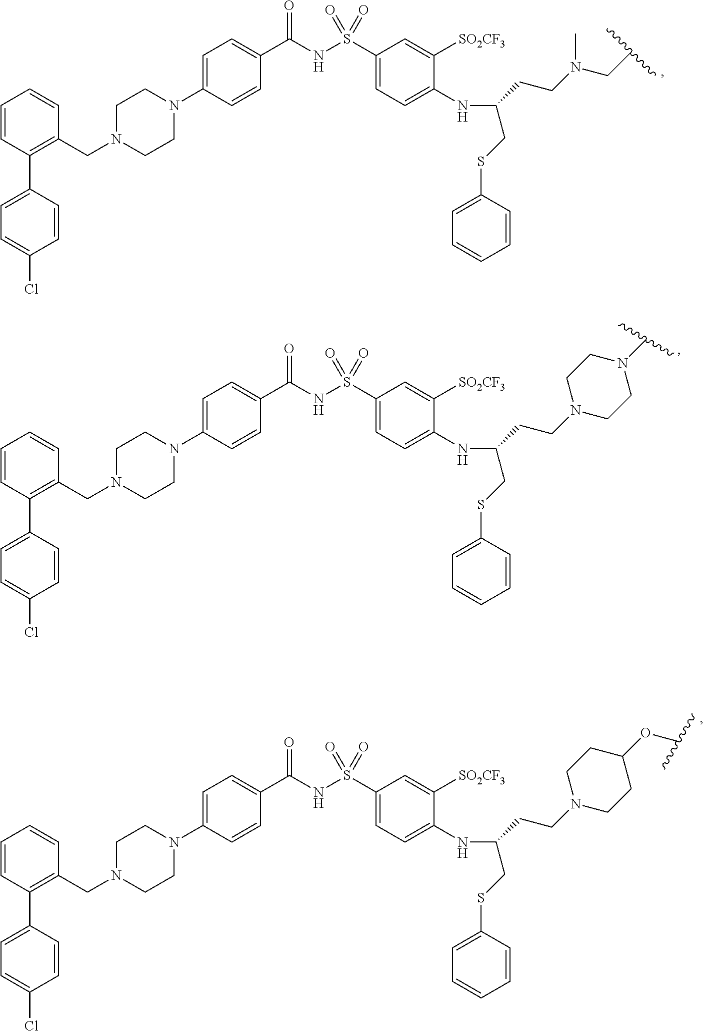 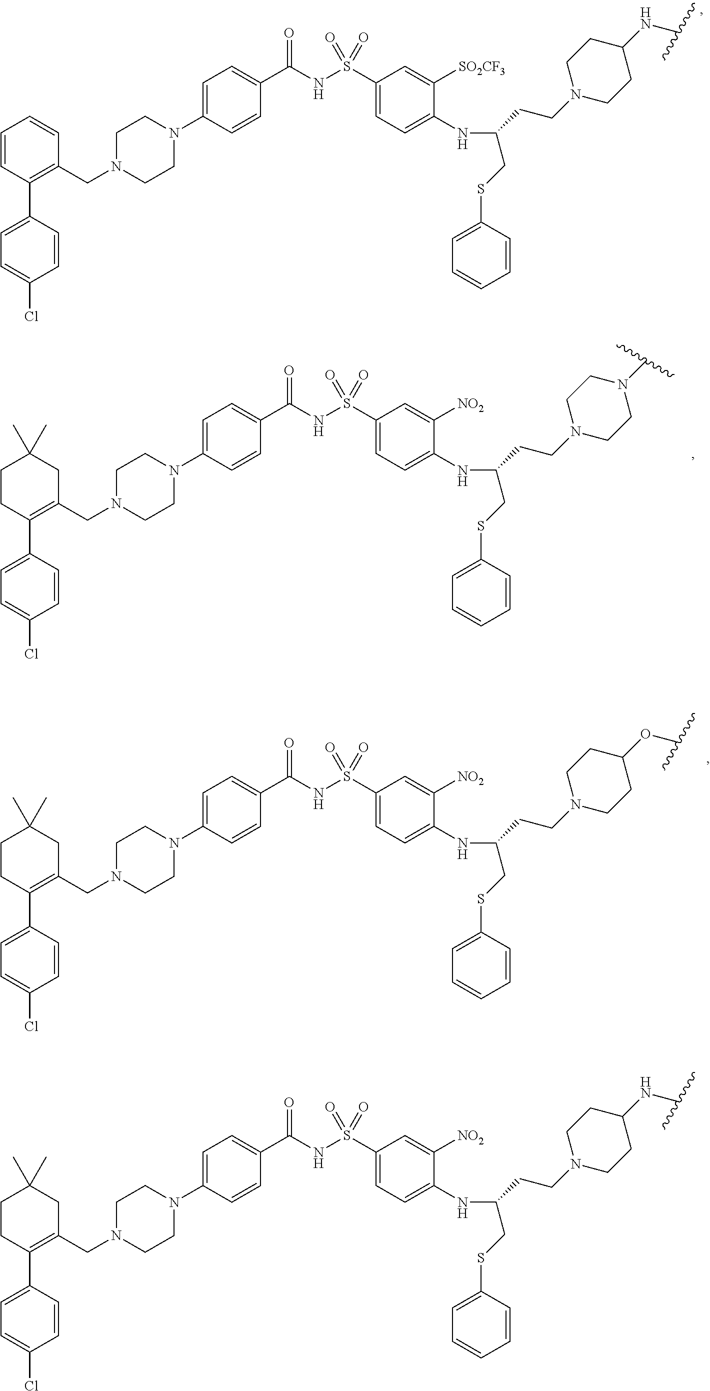 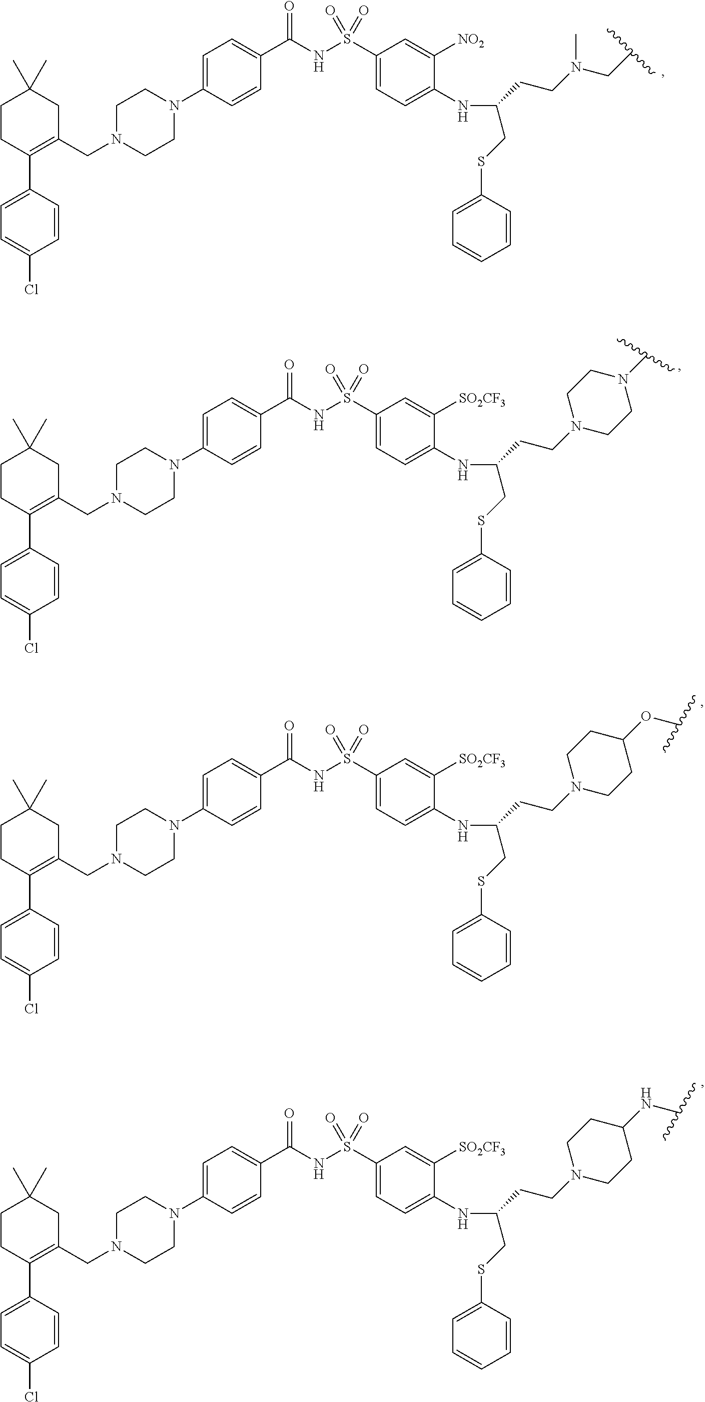 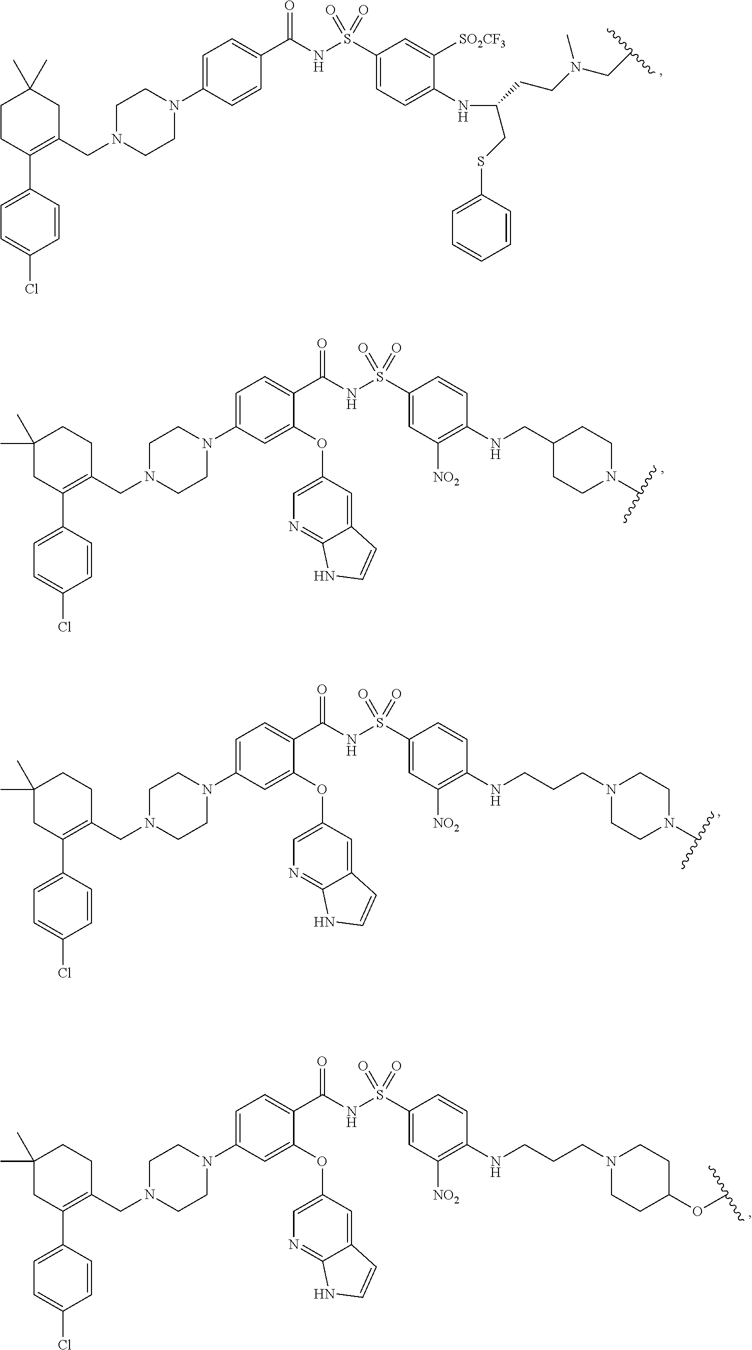 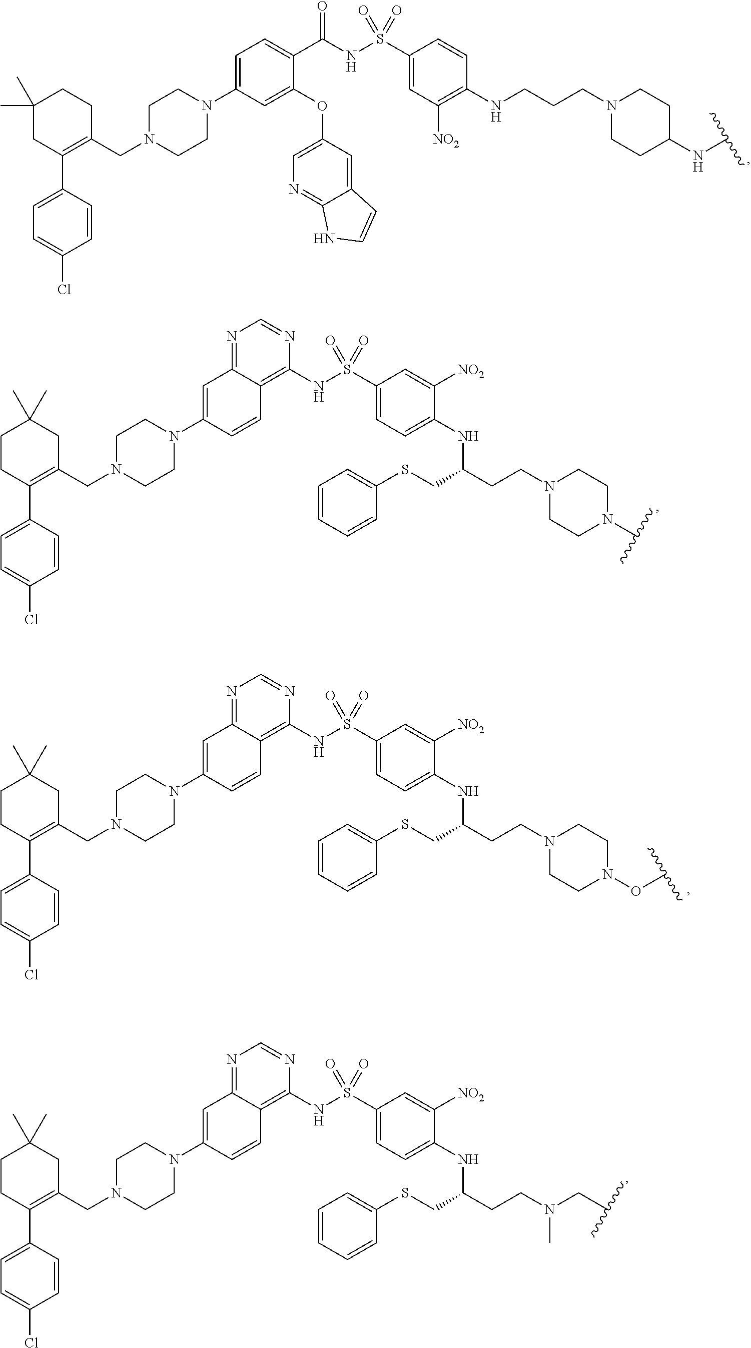 [SMILES: *NC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *ON1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-]] 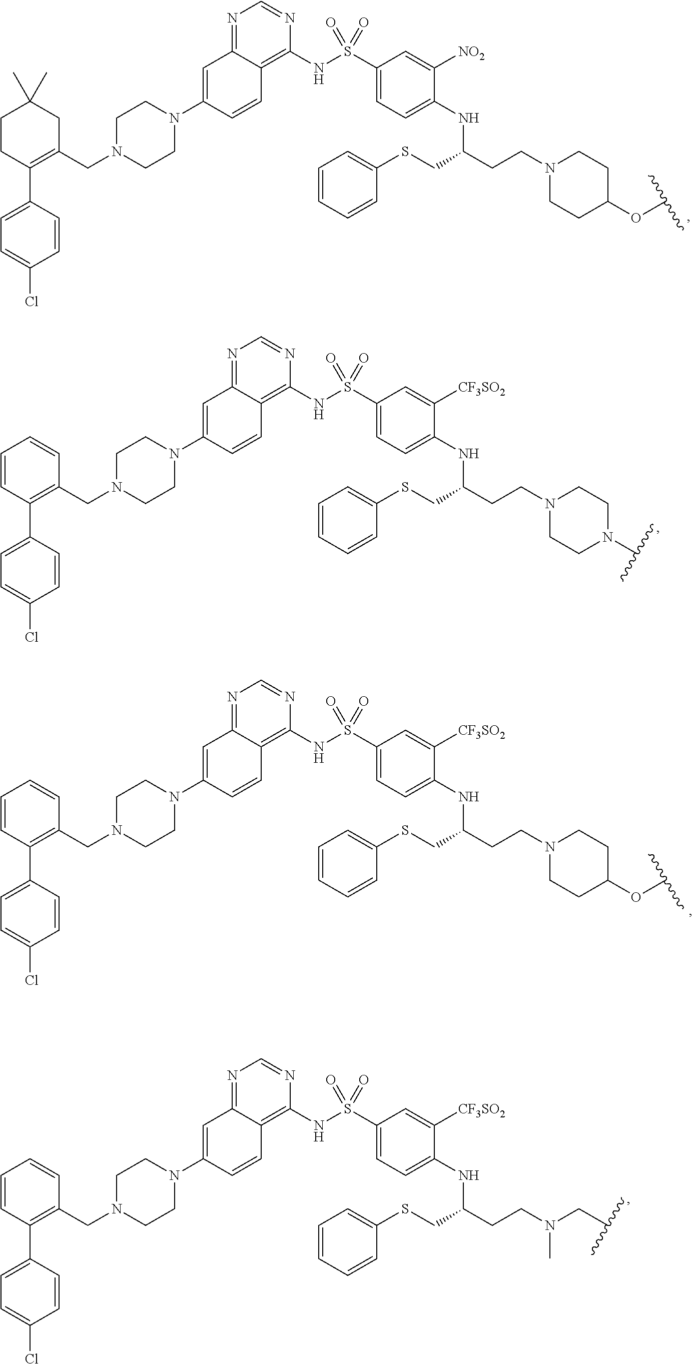 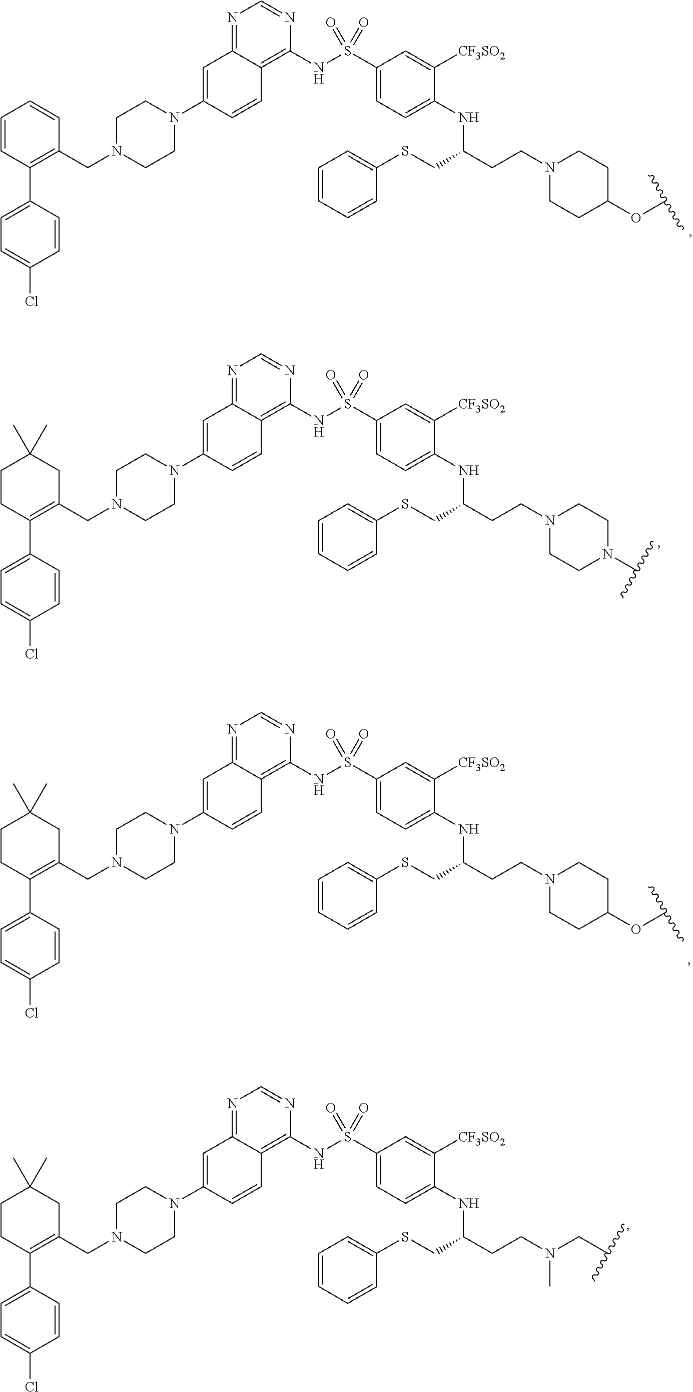 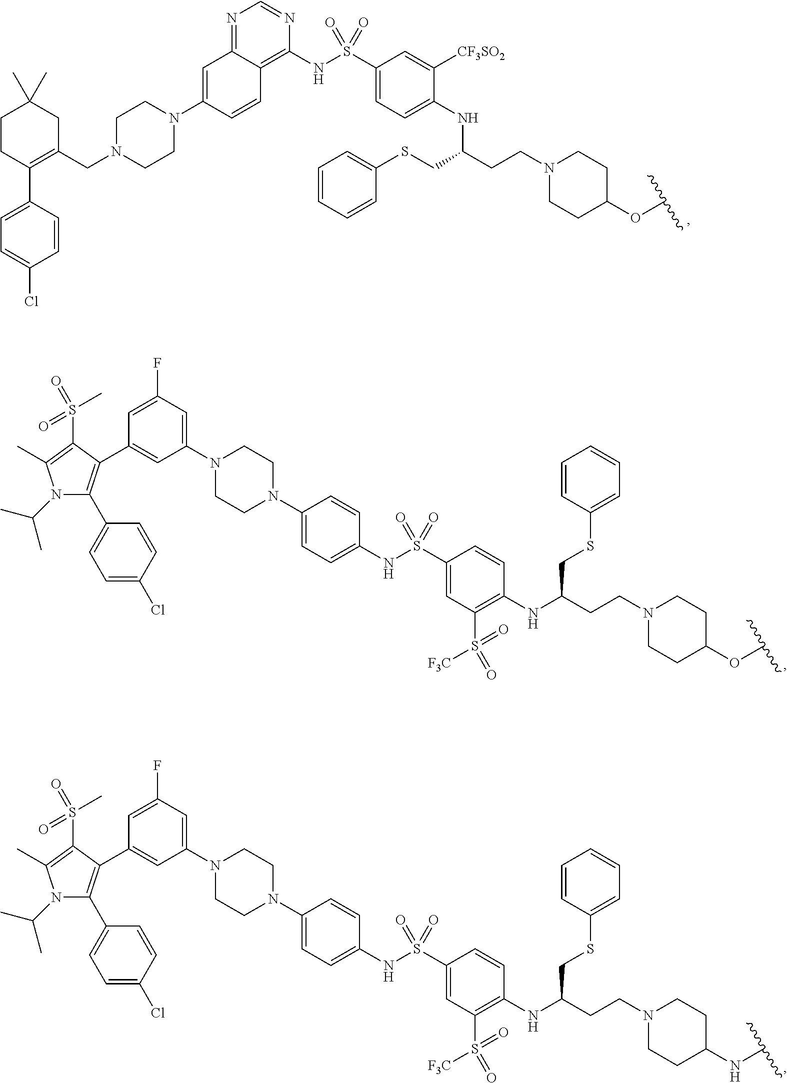 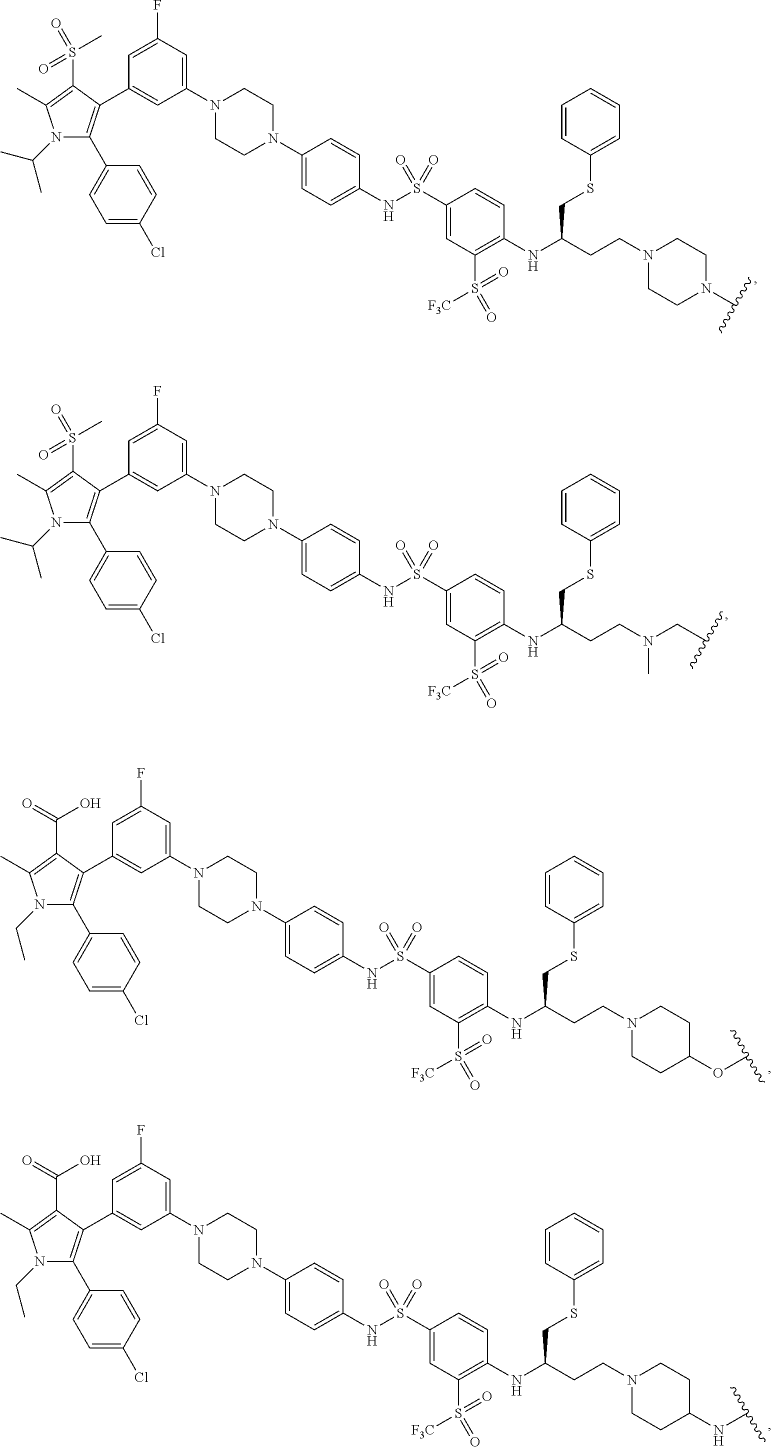 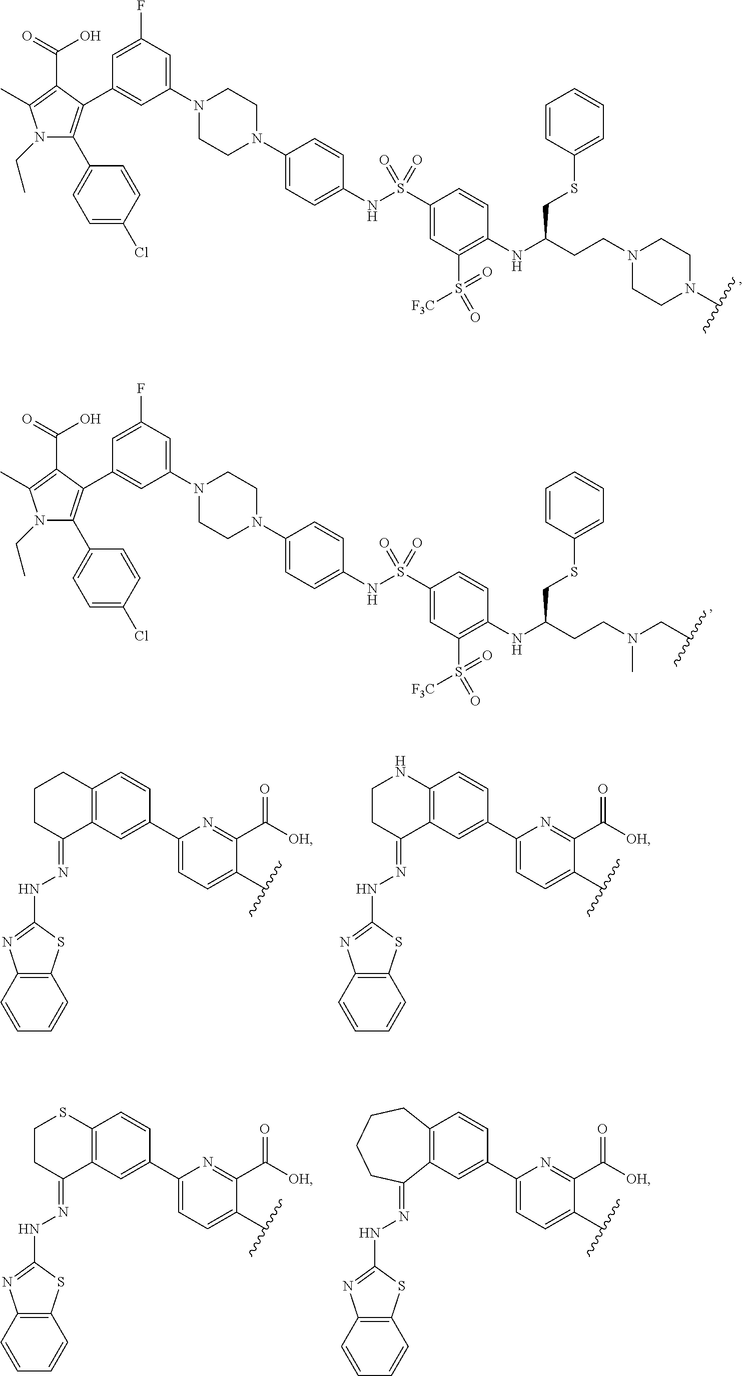 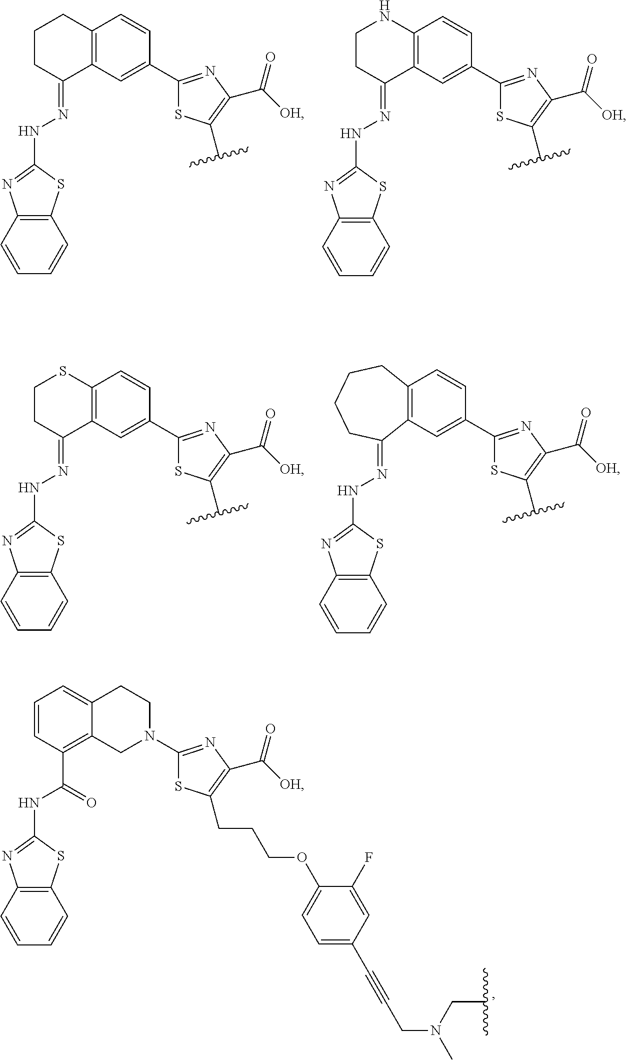 [SMILES: *c1sc(-c2ccc3c(c2)/C(=N/Nc2nc4ccccc4s2)CCC3)nc1C(=O)O ; *c1sc(-c2ccc3c(c2)/C(=N/Nc2nc4ccccc4s2)CCN3)nc1C(=O)O ; *c1sc(-c2ccc3c(c2)/C(=N/Nc2nc4ccccc4s2)CCS3)nc1C(=O)O ; *c1sc(-c2ccc3c(c2)/C(=N/Nc2nc4ccccc4s2)CCCC3)nc1C(=O)O ; *CN(C)CC#Cc1ccc(OCCCc2sc(N3CCc4cccc(C(=O)Nc5nc6ccccc6s5)c4C3)nc2C(=O)O)c(F)c1] 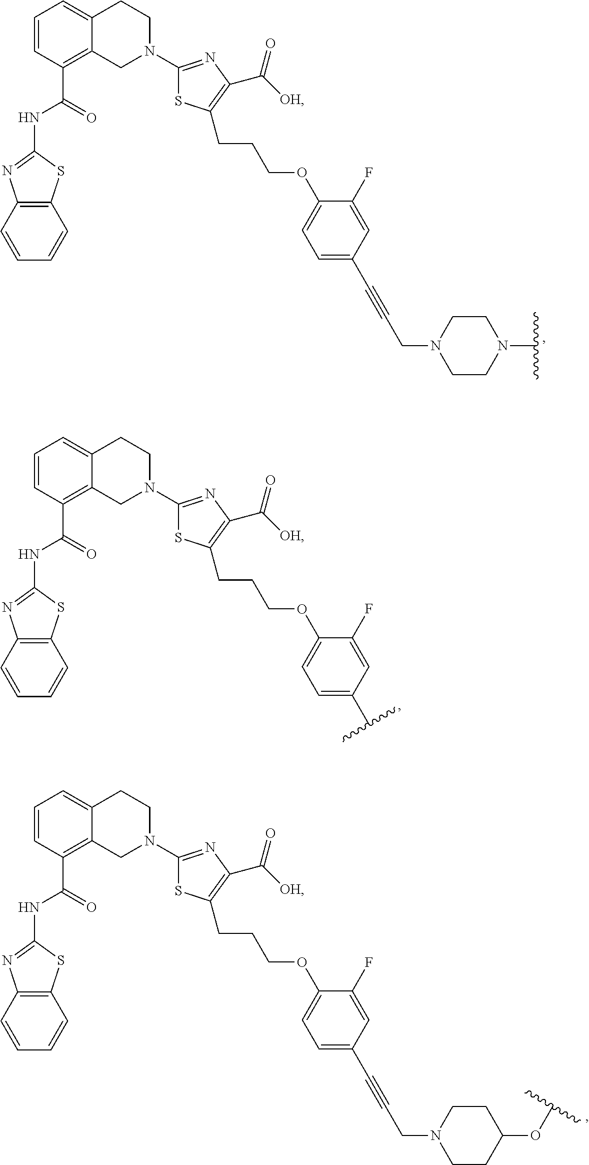 [SMILES: *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1] 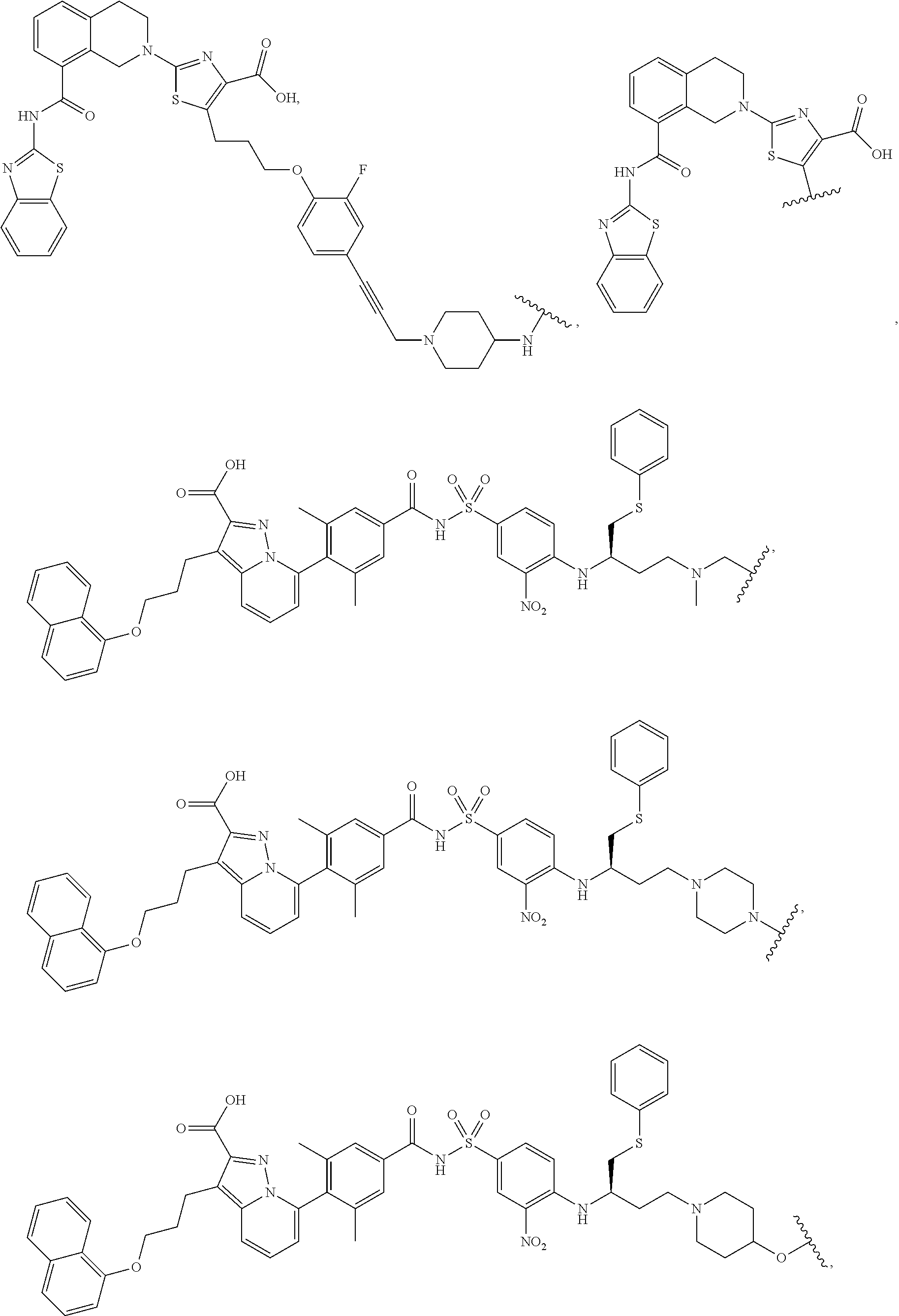 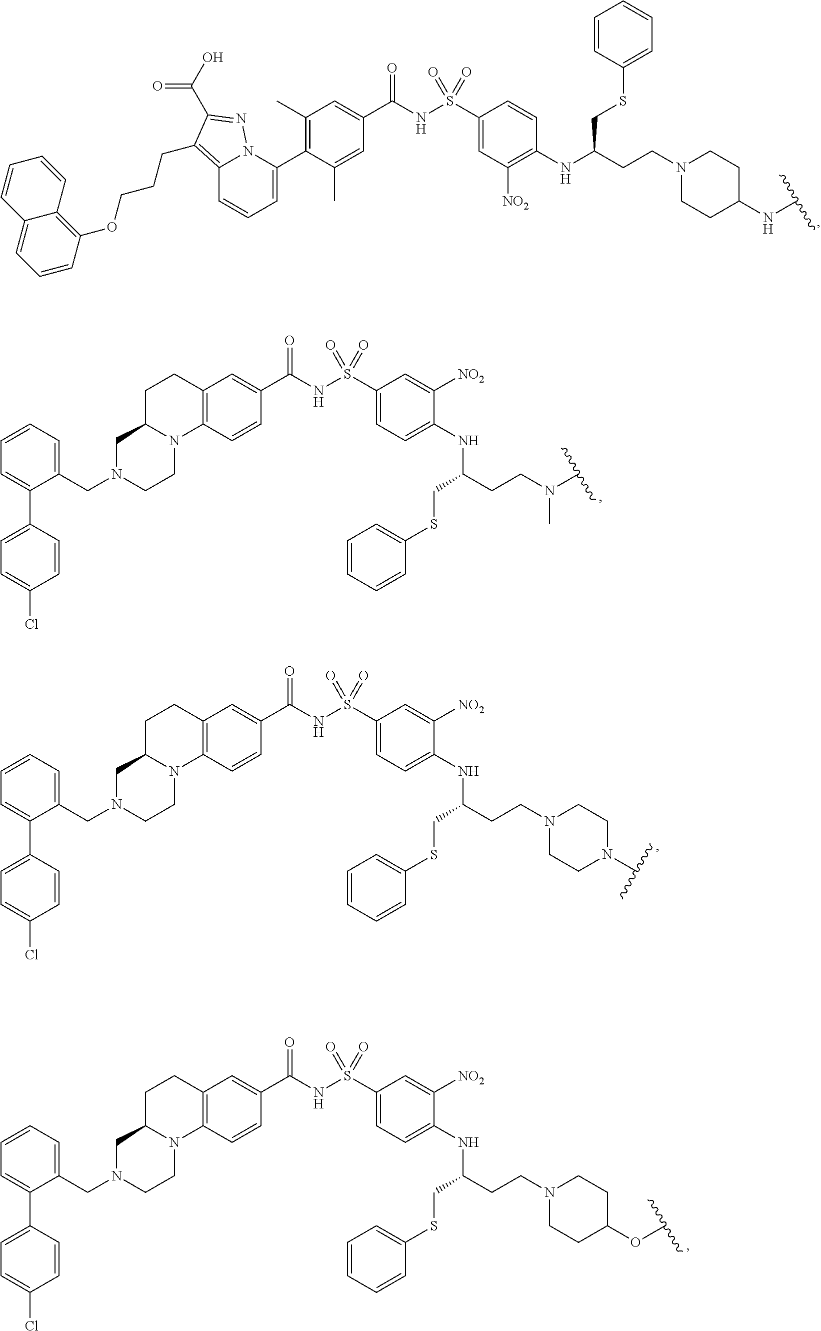 [SMILES: *NC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *N(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1] 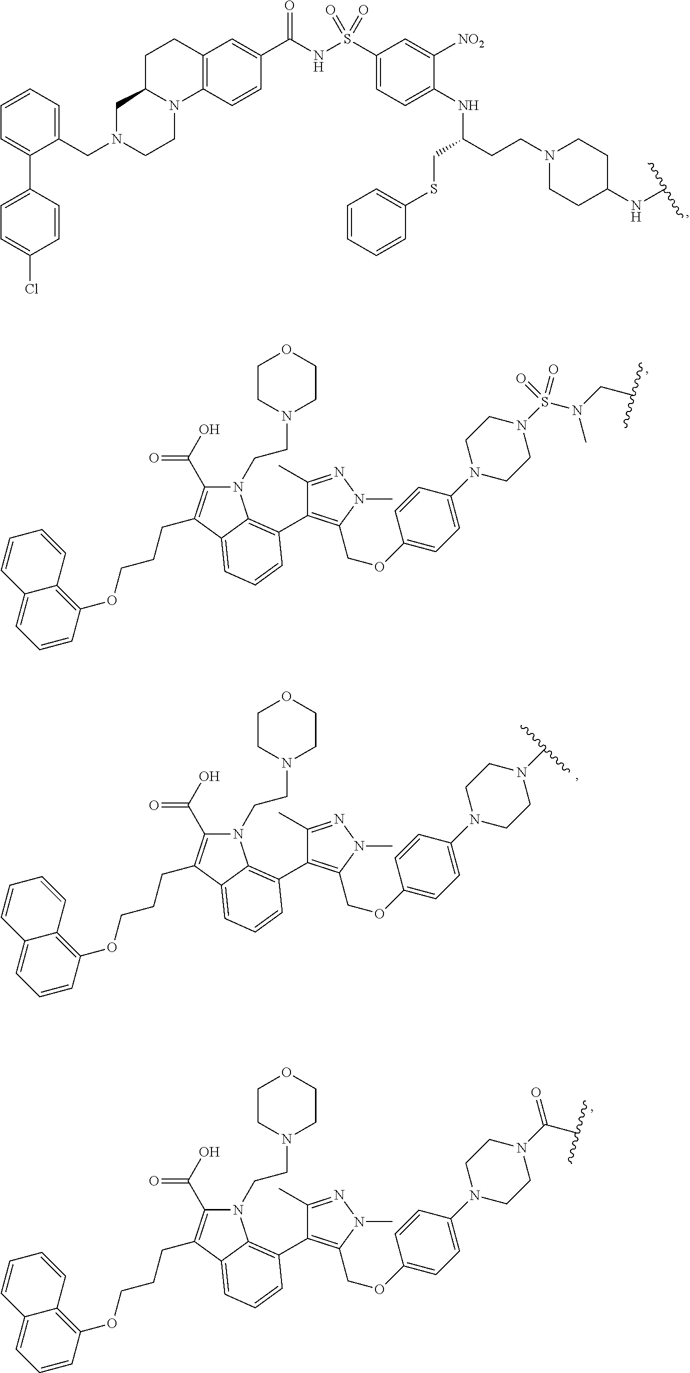 [SMILES: *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1] 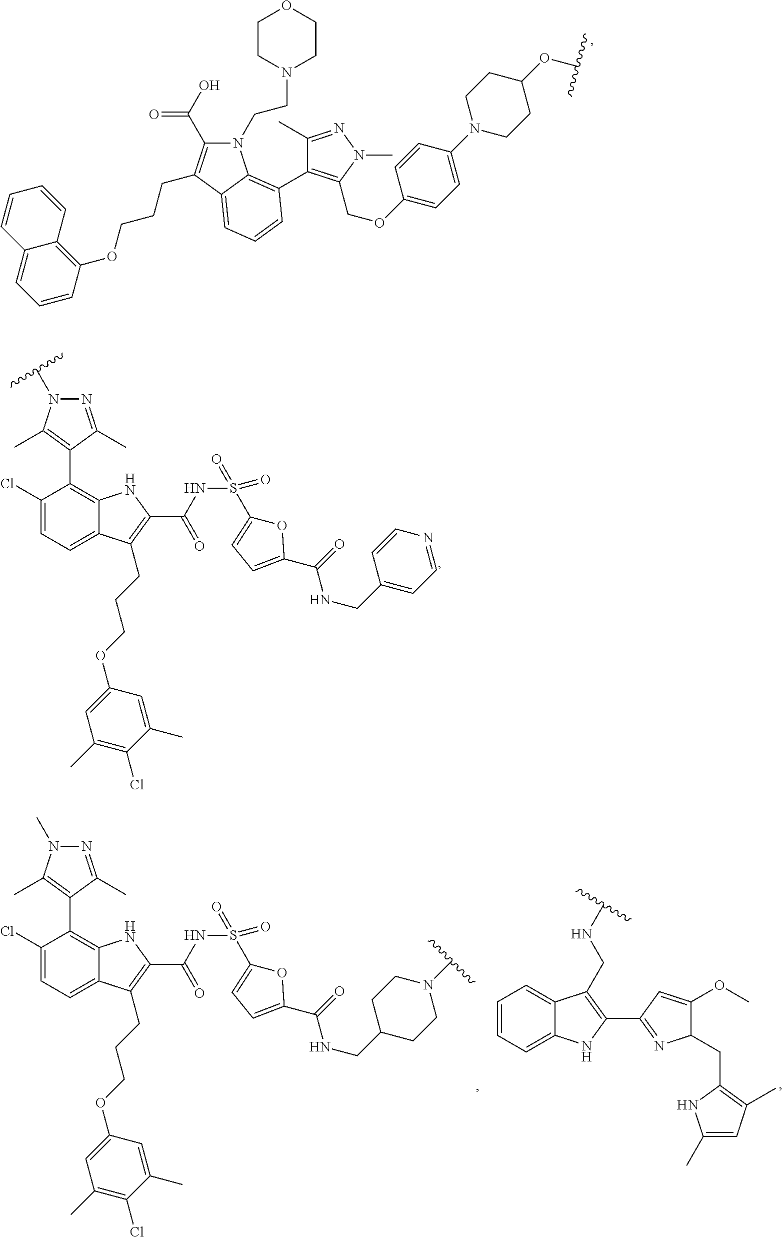 [SMILES: *OC1CCN(c2ccc(OCc3c(-c4cccc5c(CCCOc6cccc7ccccc67)c(C(=O)O)n(CCN6CCOCC6)c45)c(C)nn3C)cc2)CC1 ; *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=NC(Cc3[nH]c(C)cc3C)C(OC)=C2)[nH]c2ccccc12] 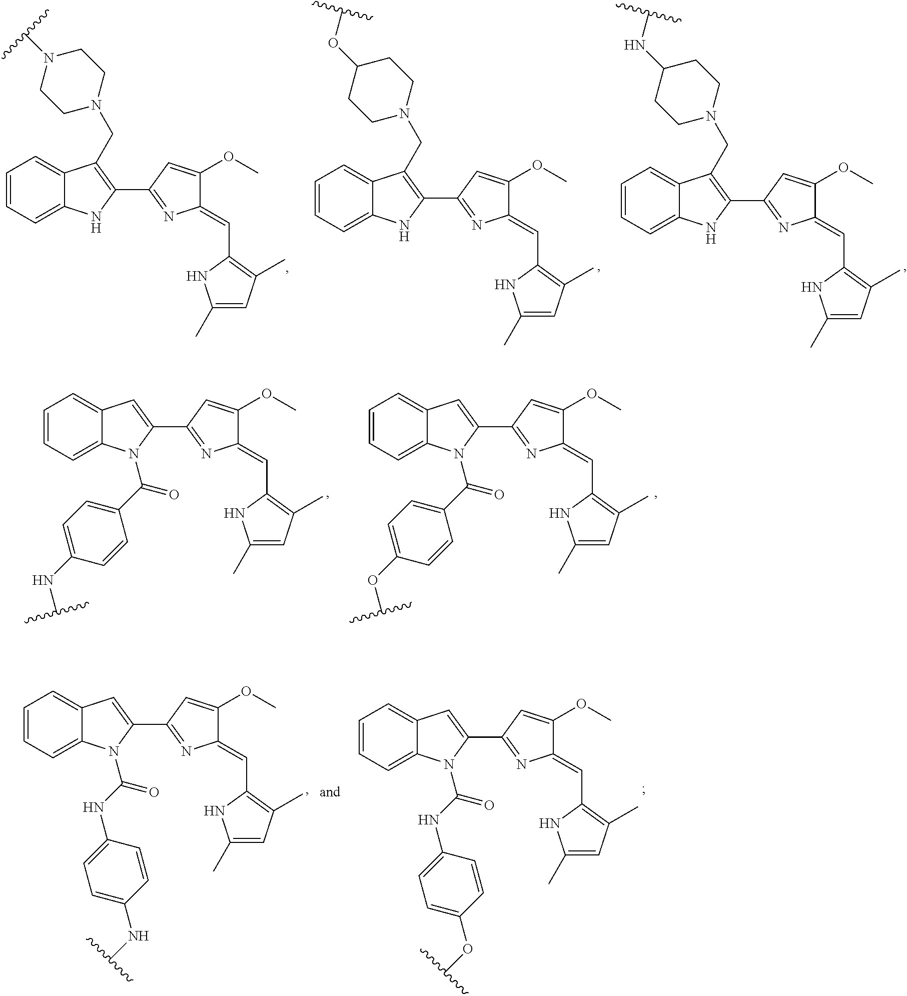
R³ is absent, a bond, or a substituted or unsubstituted C₁-C₁₀ alkyl;
A is absent, a bond, a substituted or unsubstituted C₁-C₆ aryl, a substituted or unsubstituted C₁-C₆cycloalkyl, or a substituted or unsubstituted C₁-C₆ heterocyclic group;
R⁴ is a bond or a substituted or unsubstituted C₁-C₁₀ alky;
n is an integer from 0 to 5,
R² is selected from the group consisting of

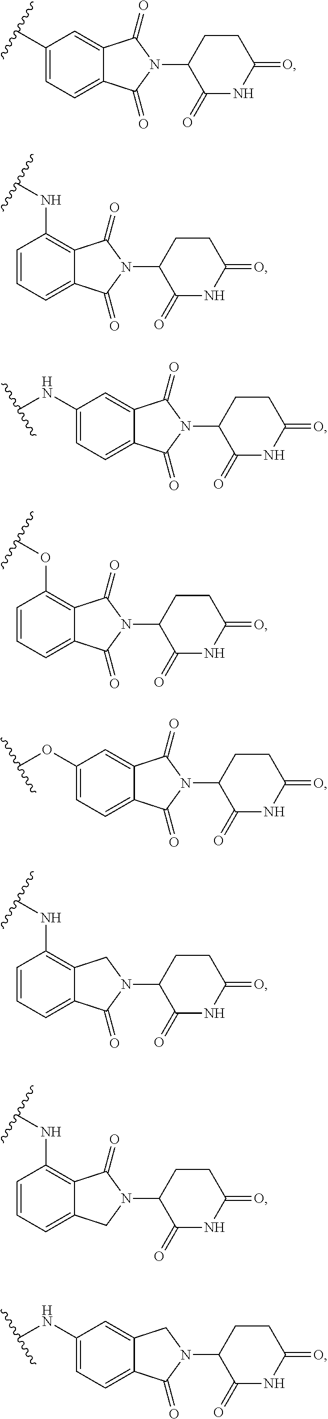

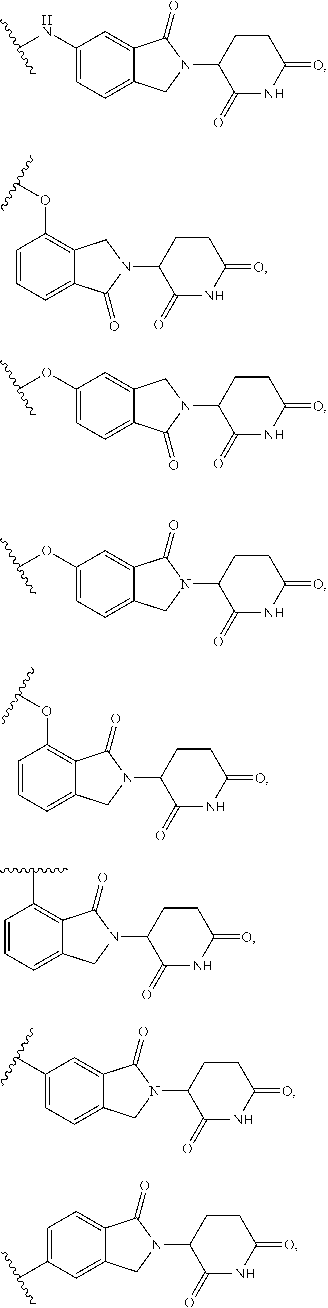

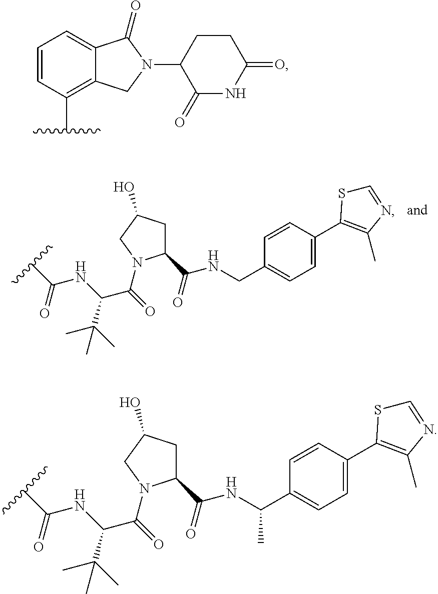 [SMILES: *c1cccc2c1CN(C1CCC(=O)NC1=O)C2=O ; *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

11. The method of claim 10, wherein R¹ is selected from the group consisting of:

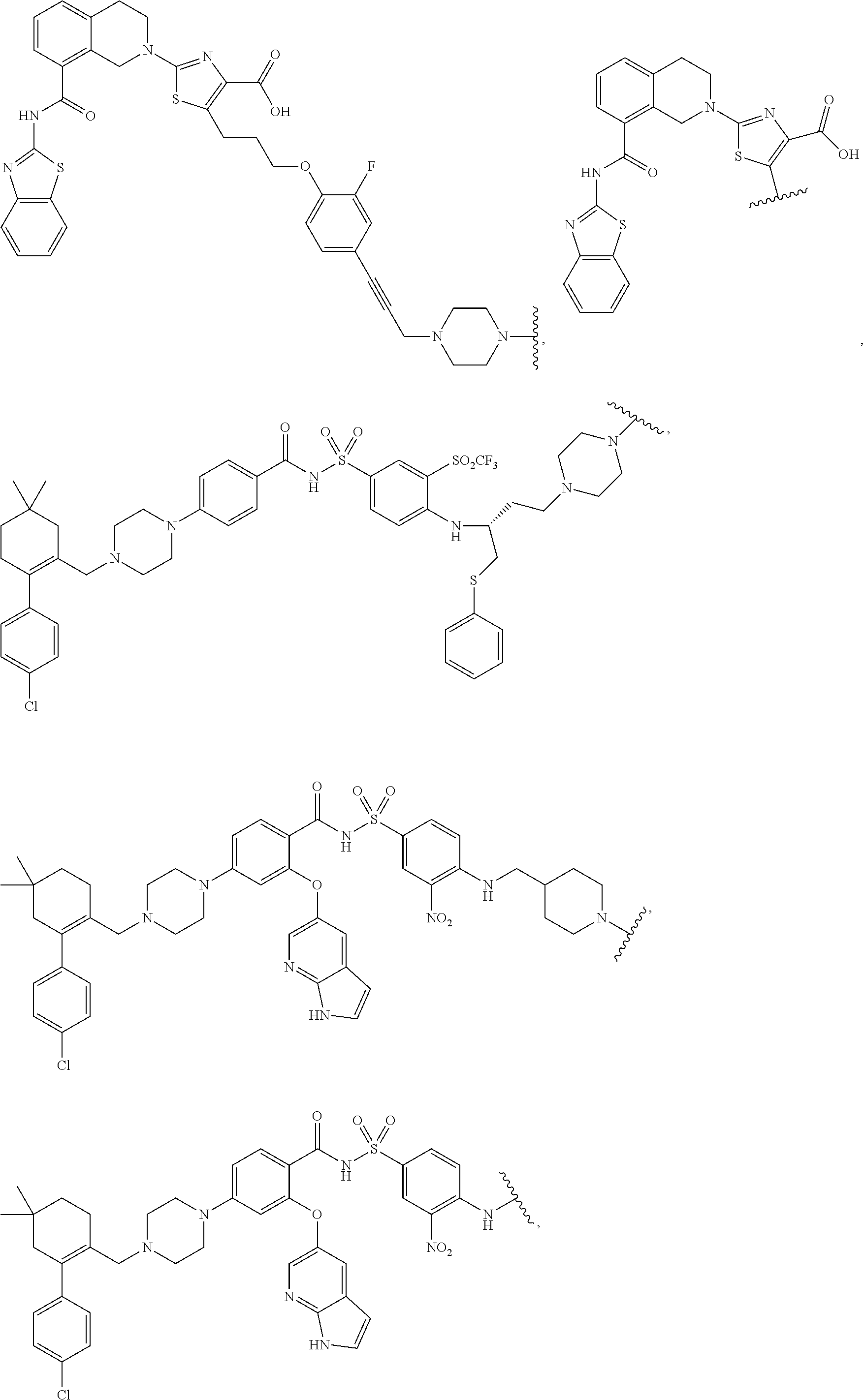

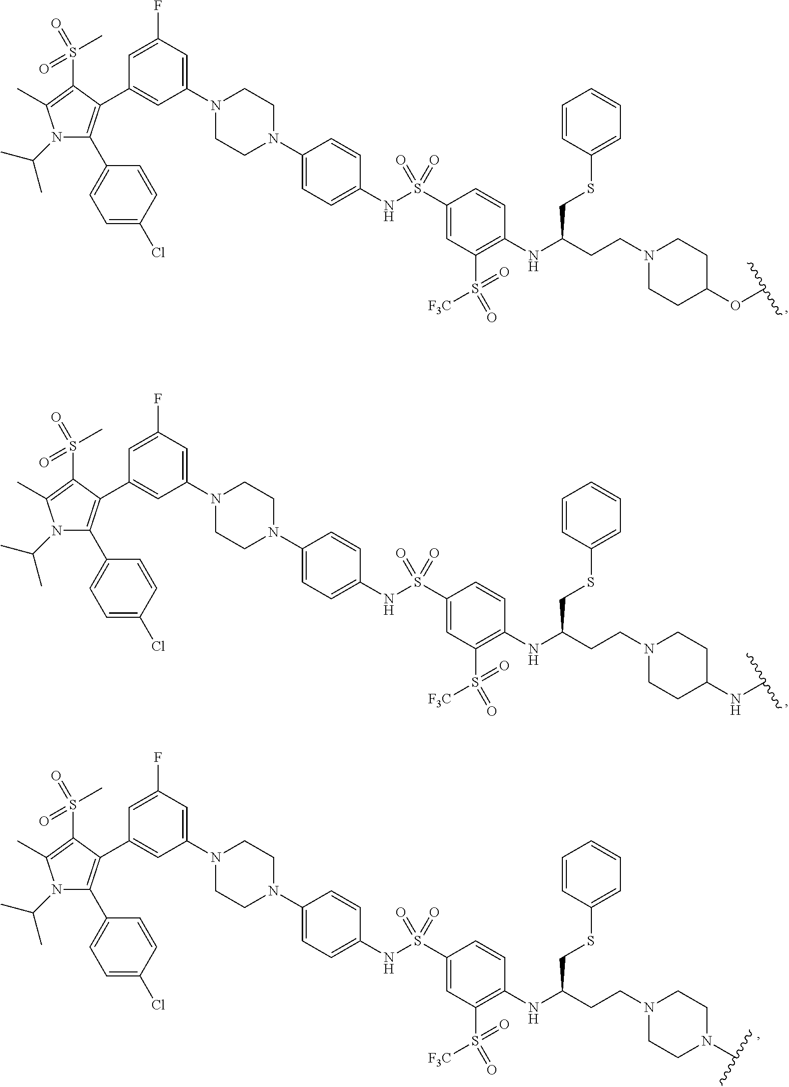 [SMILES: *OC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *N1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1]

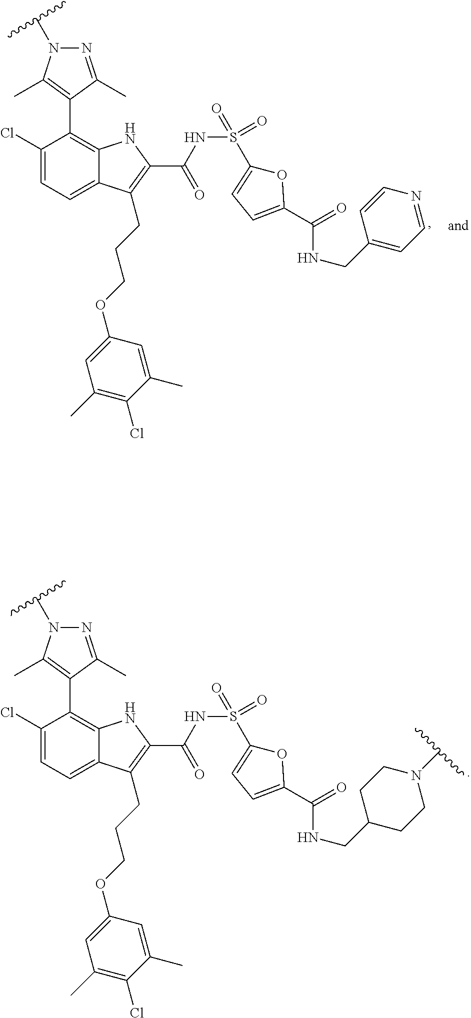 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(*)c5C)c(Cl)ccc4c3CCCOc3cc(C)c(Cl)c(C)c3)o2)CC1]

12. A method of selectively killing one or more senescent bone cells in a sample or subject in need thereof, the method comprising administering to the sample or subject a composition comprising a therapeutically effective amount of a compound comprising Formula (II): 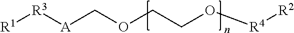
wherein
R¹ is selected from the group consisting of: 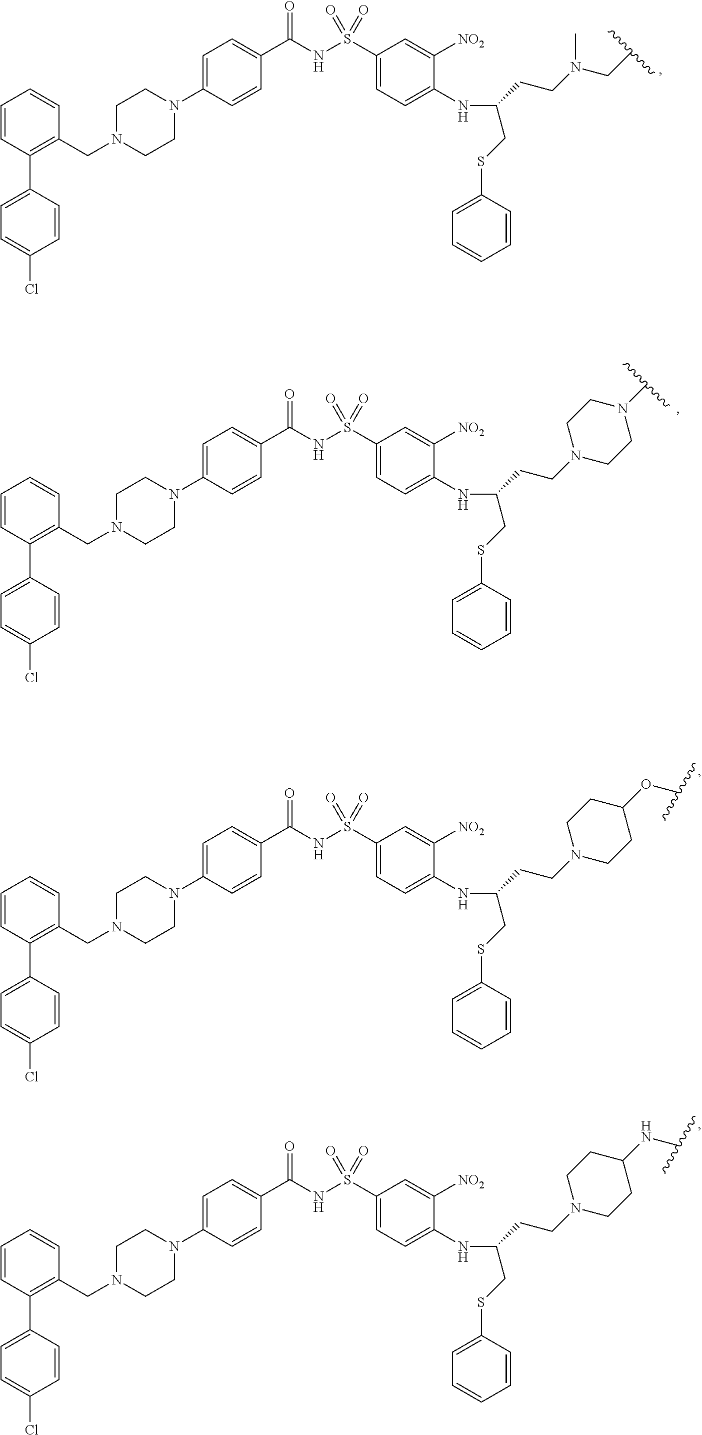 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1] 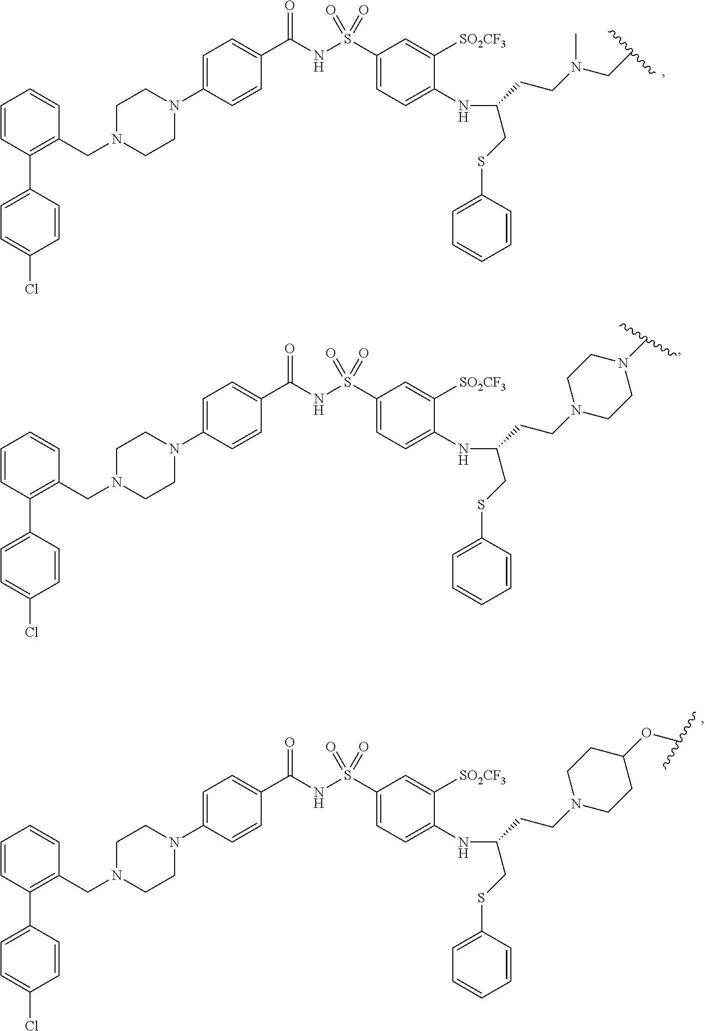 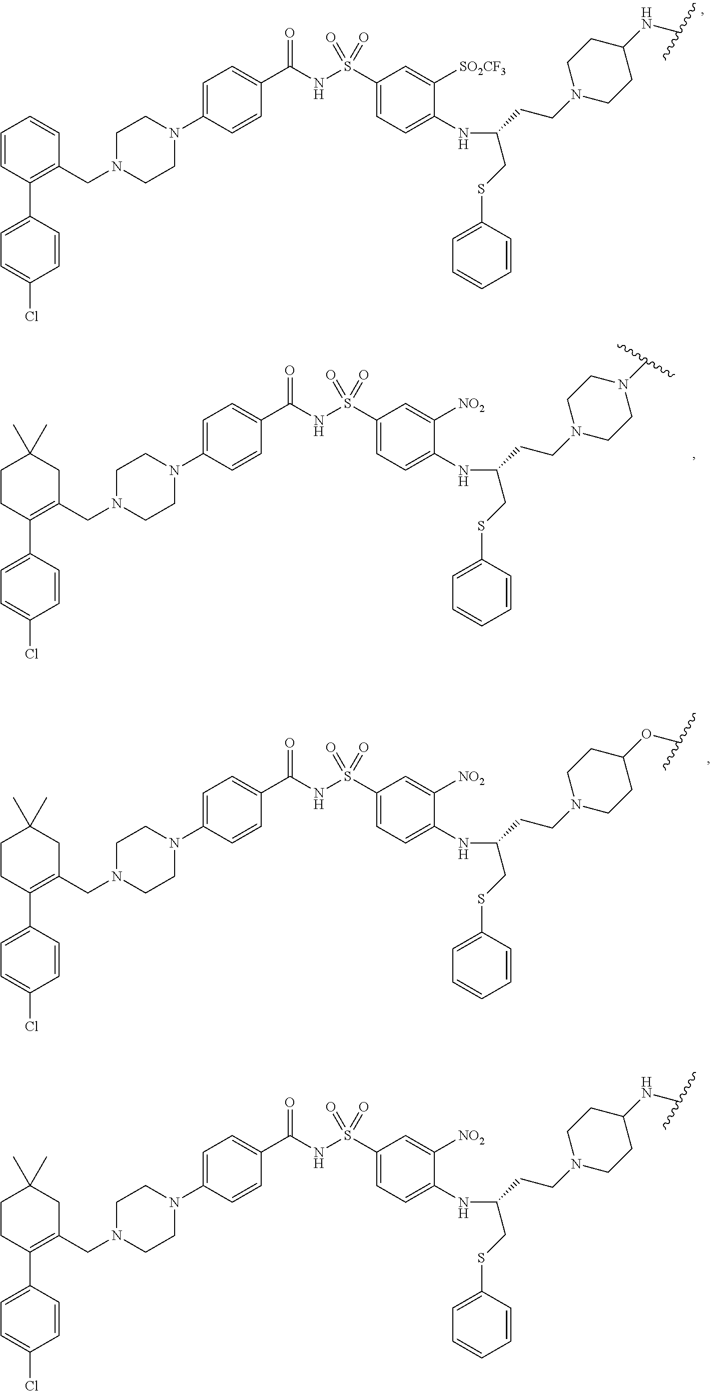 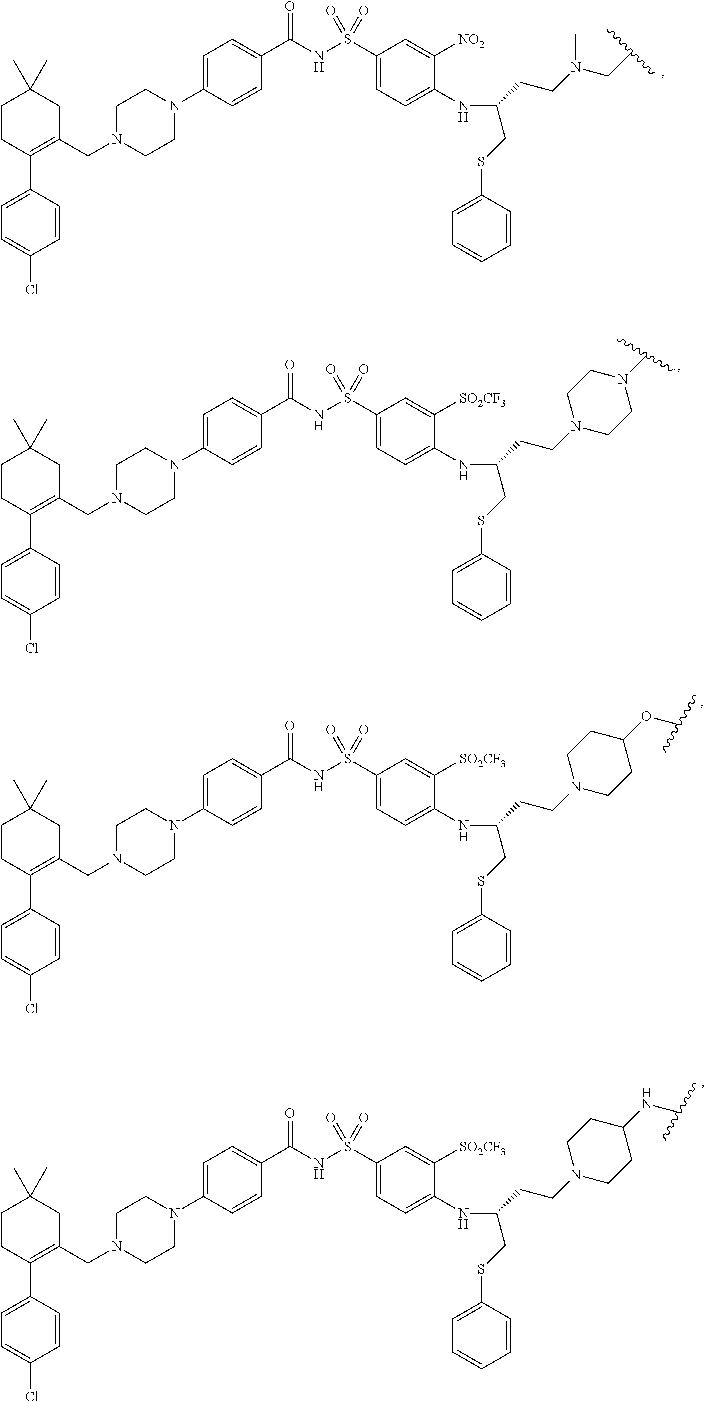 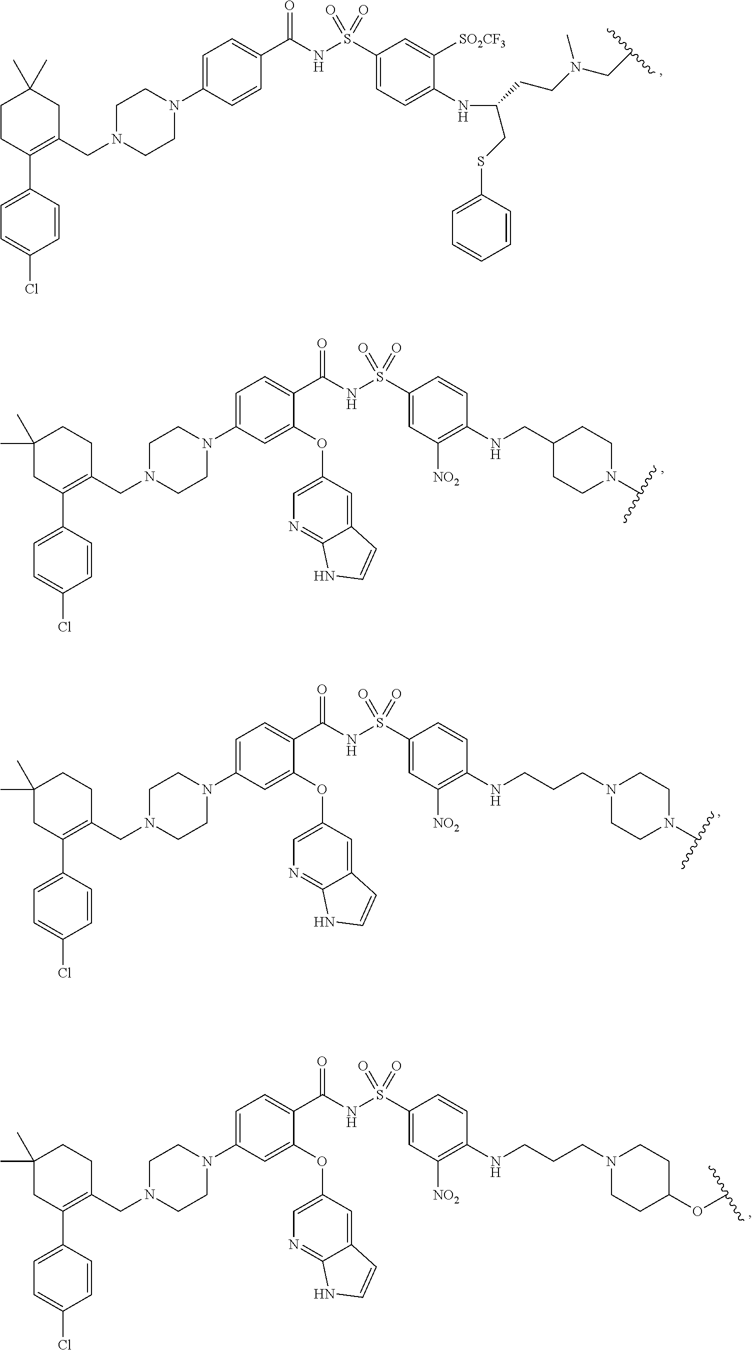 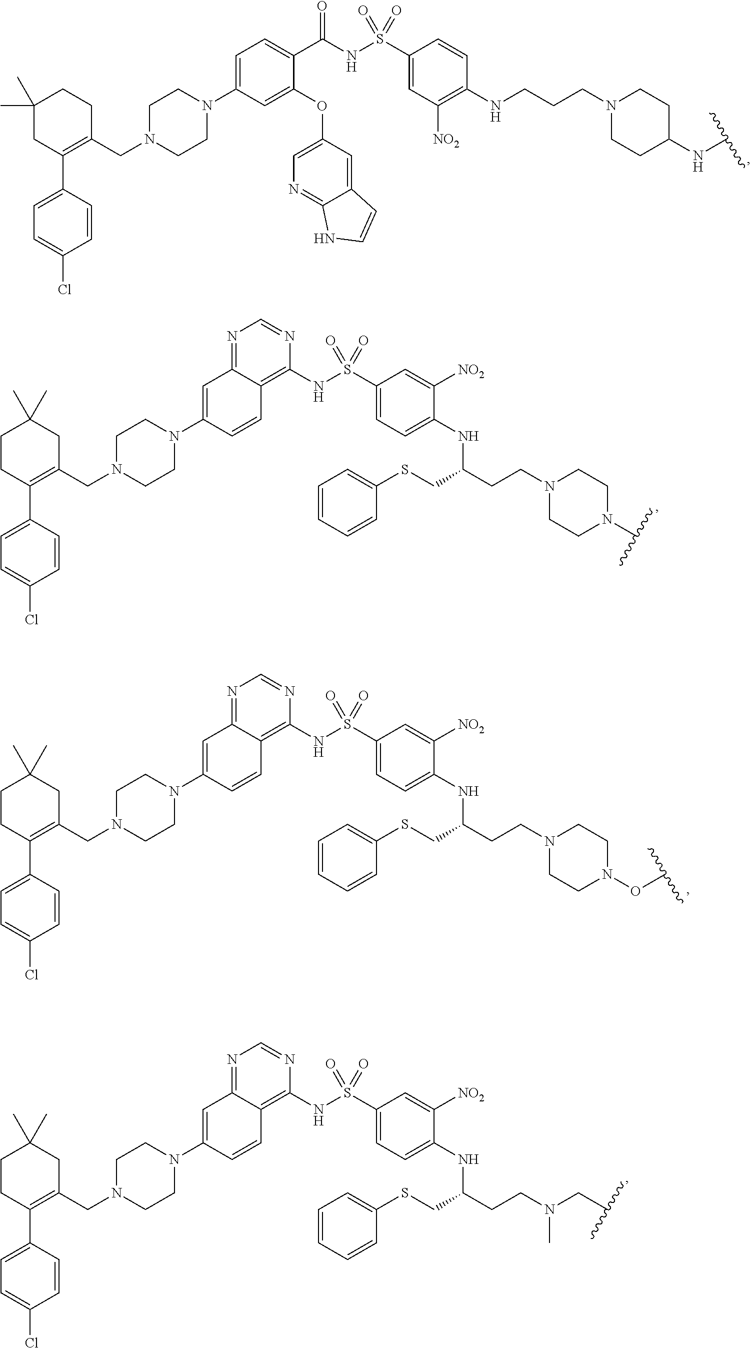 [SMILES: *NC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *ON1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-]] 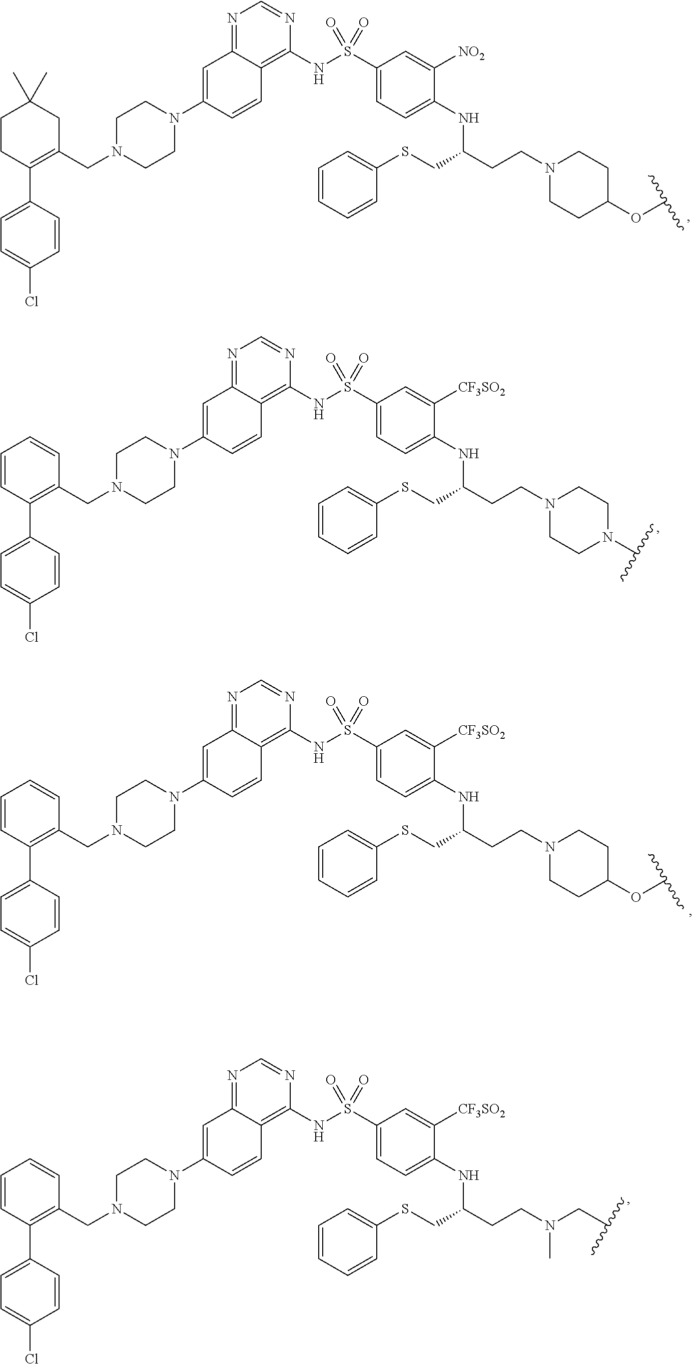 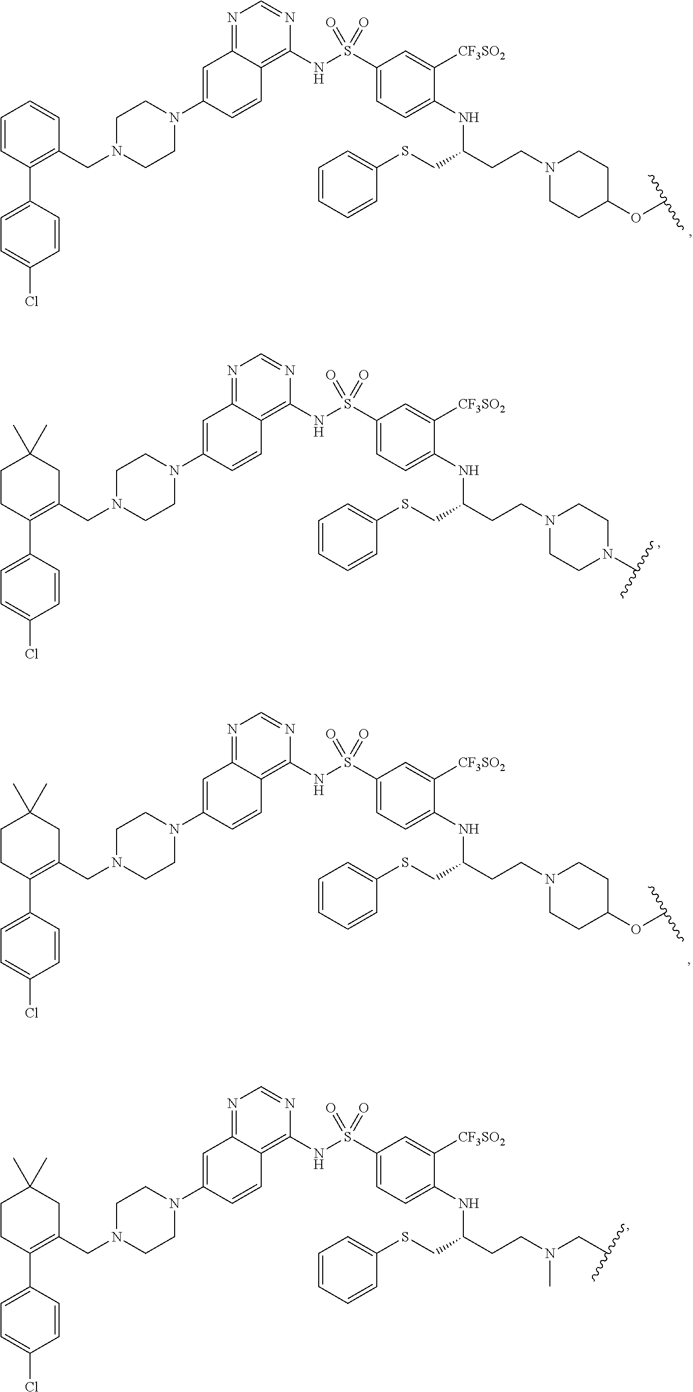 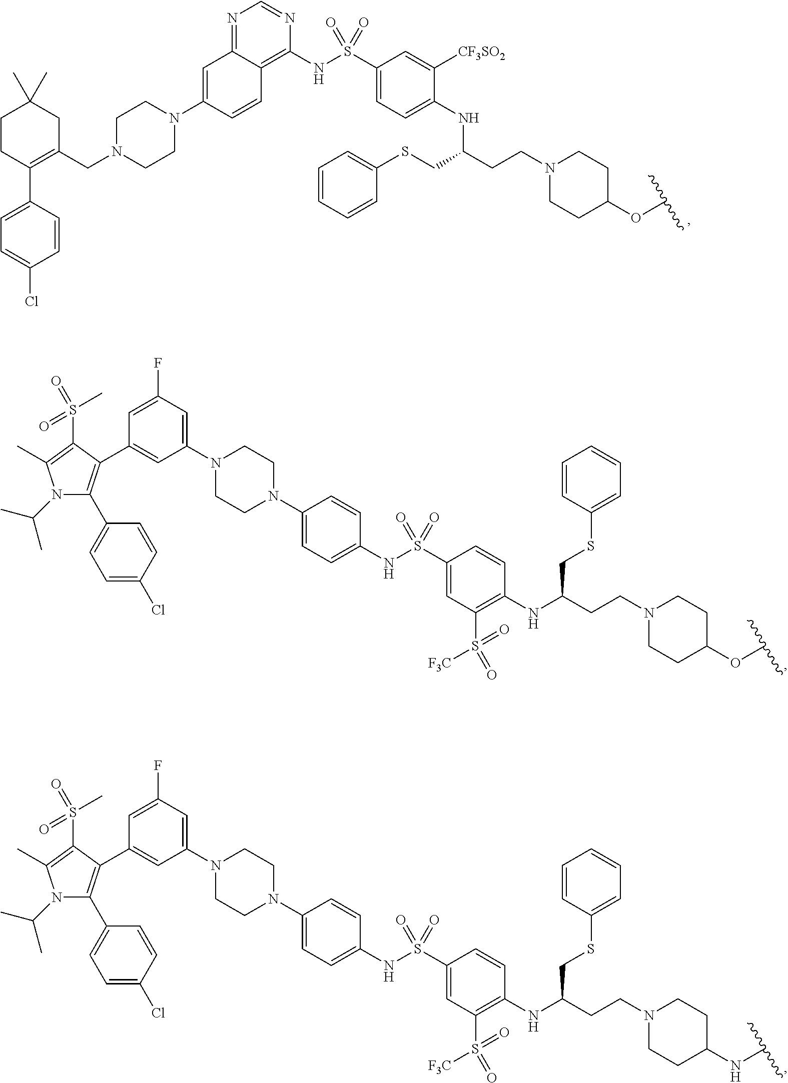 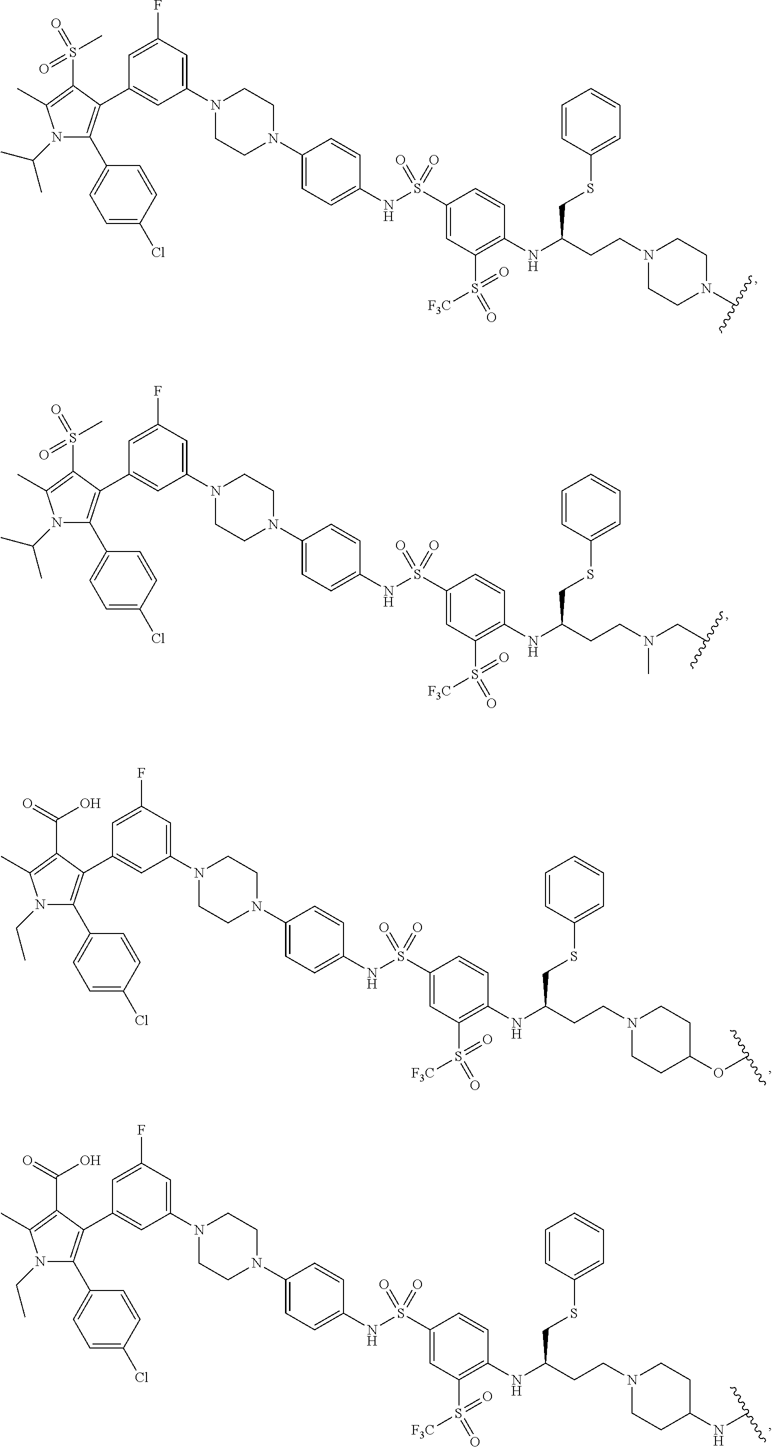 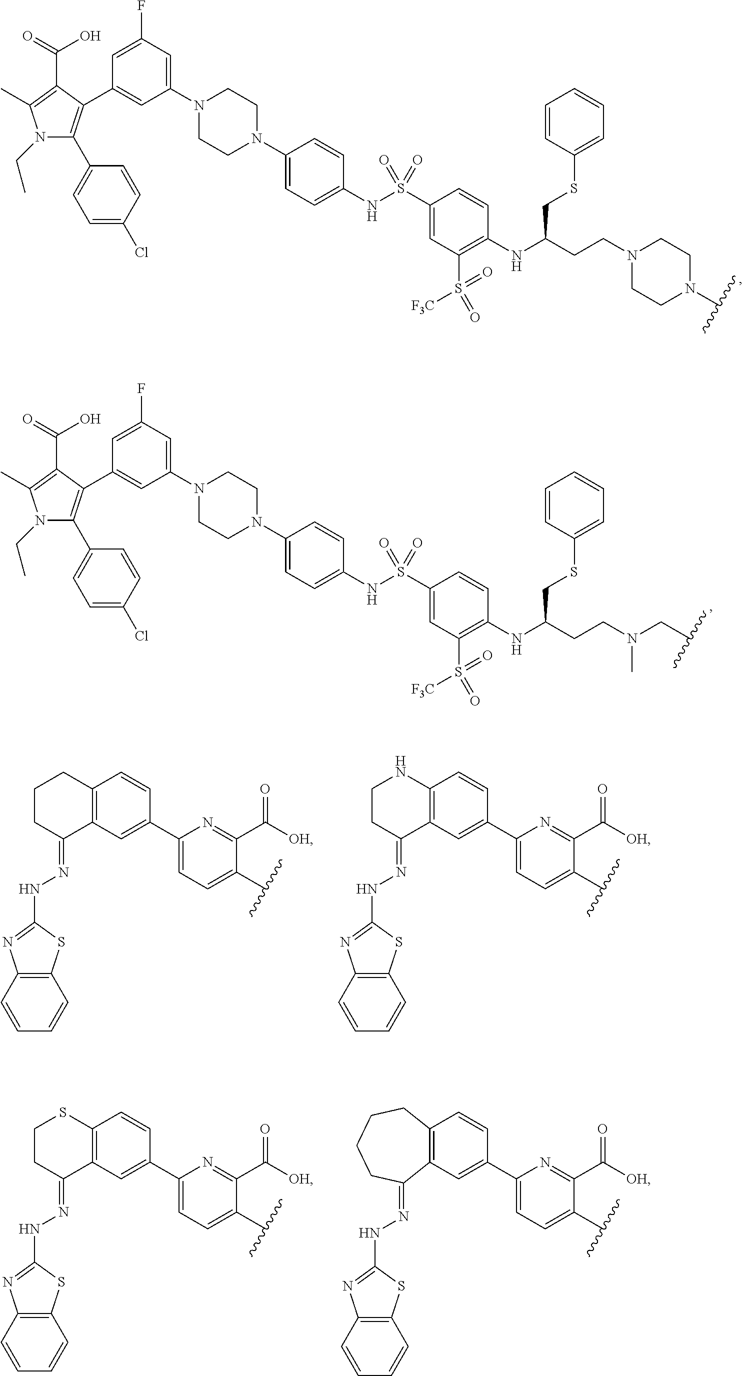 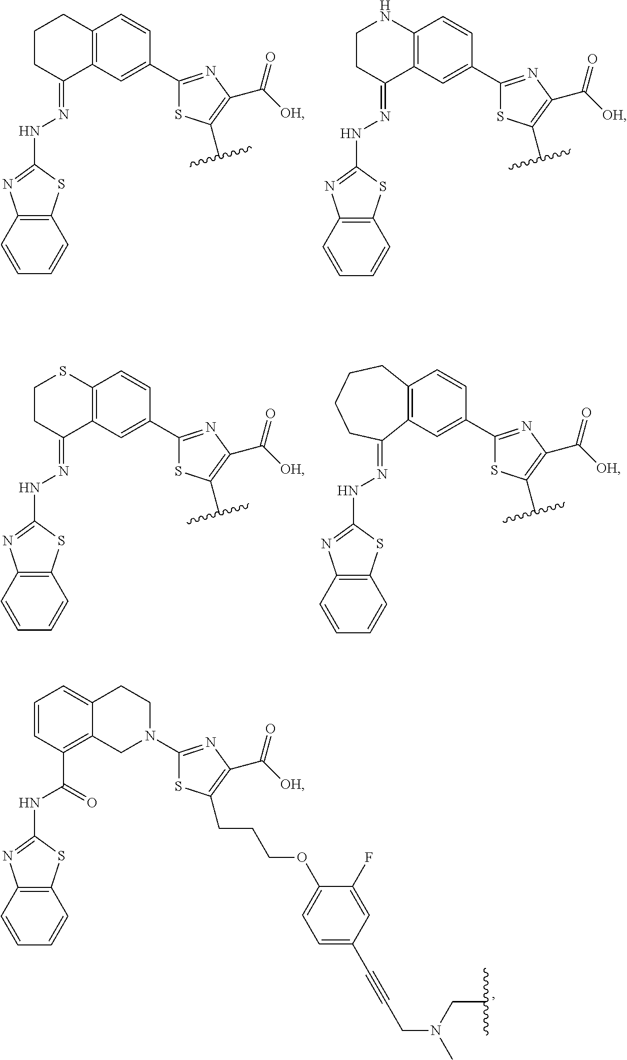 [SMILES: *c1sc(-c2ccc3c(c2)/C(=N/Nc2nc4ccccc4s2)CCC3)nc1C(=O)O ; *c1sc(-c2ccc3c(c2)/C(=N/Nc2nc4ccccc4s2)CCN3)nc1C(=O)O ; *c1sc(-c2ccc3c(c2)/C(=N/Nc2nc4ccccc4s2)CCS3)nc1C(=O)O ; *c1sc(-c2ccc3c(c2)/C(=N/Nc2nc4ccccc4s2)CCCC3)nc1C(=O)O ; *CN(C)CC#Cc1ccc(OCCCc2sc(N3CCc4cccc(C(=O)Nc5nc6ccccc6s5)c4C3)nc2C(=O)O)c(F)c1] 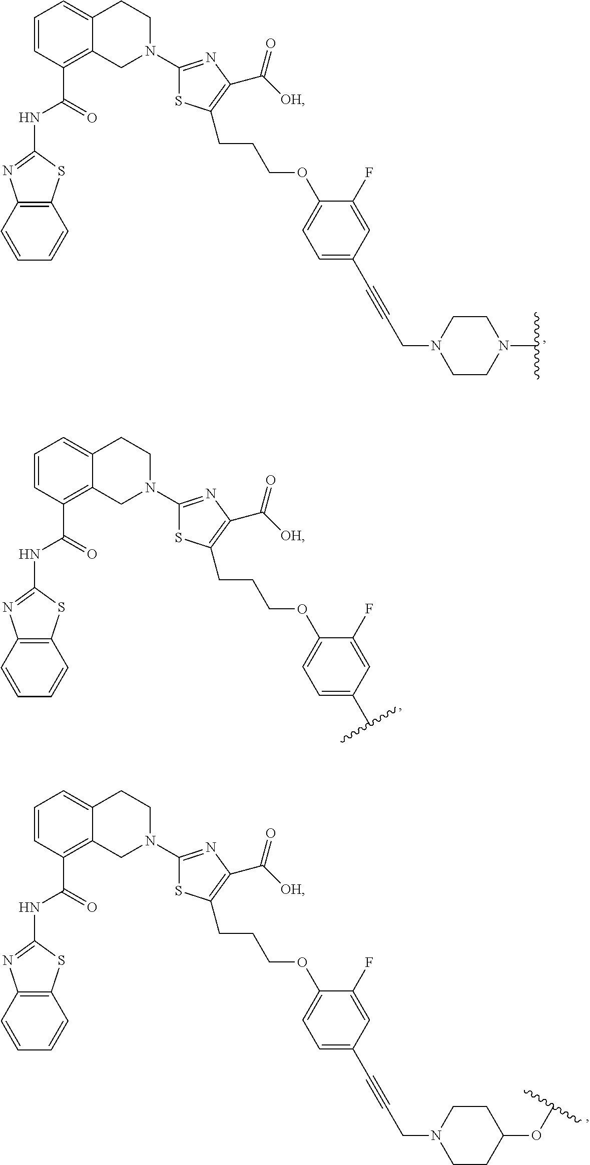 [SMILES: *N1CCN(CC#Cc2ccc(OCCCc3sc(N4CCc5cccc(C(=O)Nc6nc7ccccc7s6)c5C4)nc3C(=O)O)c(F)c2)CC1 ; *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1] 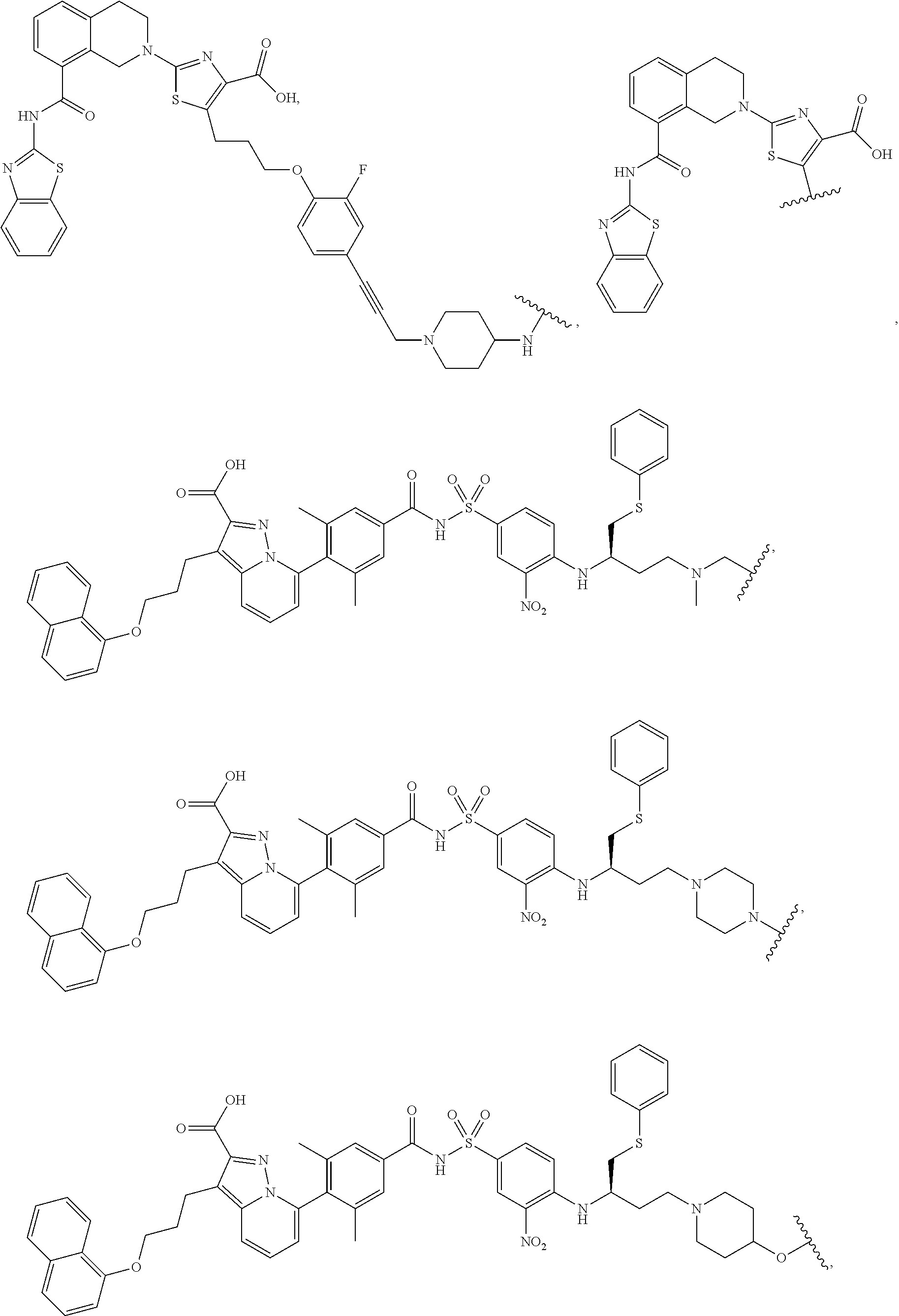 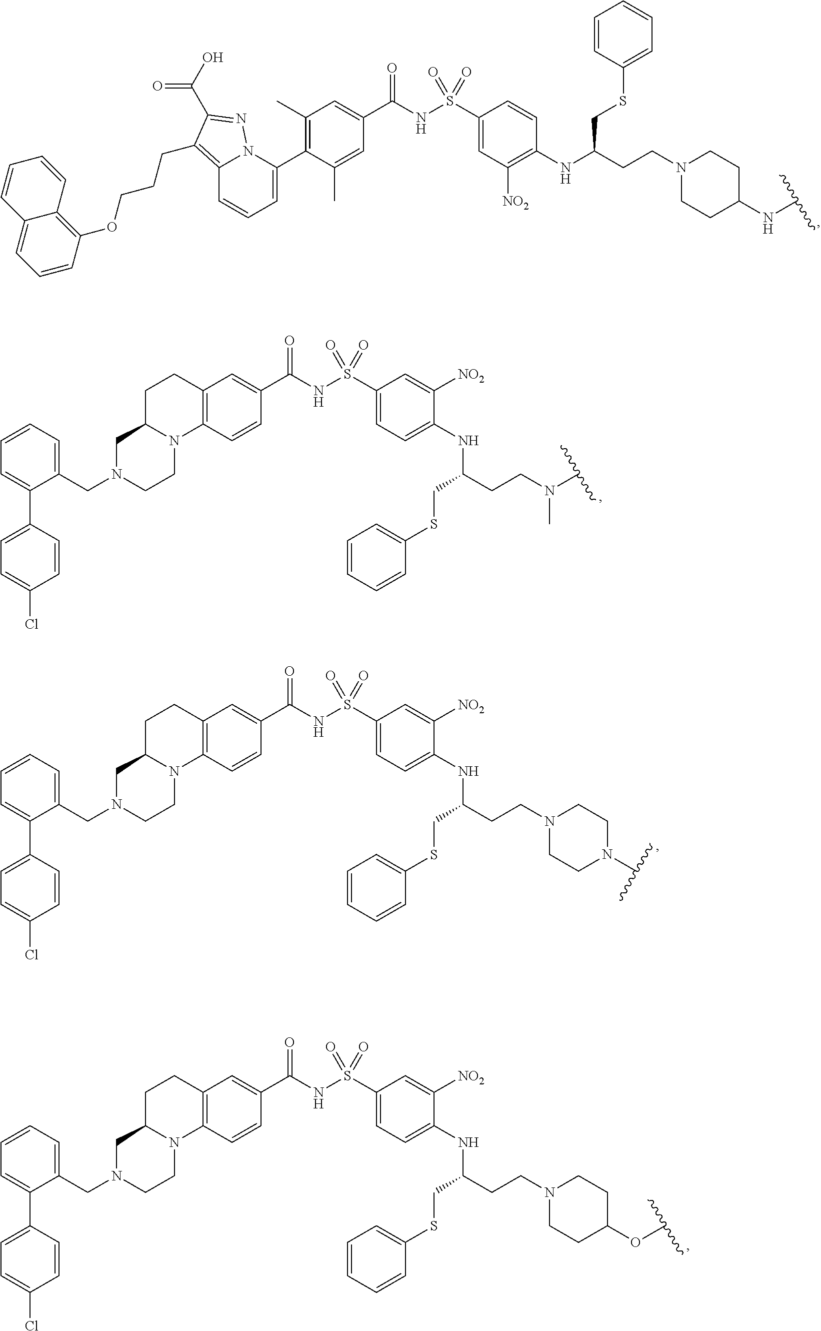 [SMILES: *NC1CCN(CC[C@H](CSc2ccccc2)Nc2ccc(S(=O)(=O)NC(=O)c3cc(C)c(-c4cccc5c(CCCOc6cccc7ccccc67)c(C(=O)O)nn45)c(C)c3)cc2[N+](=O)[O-])CC1 ; *N(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1] 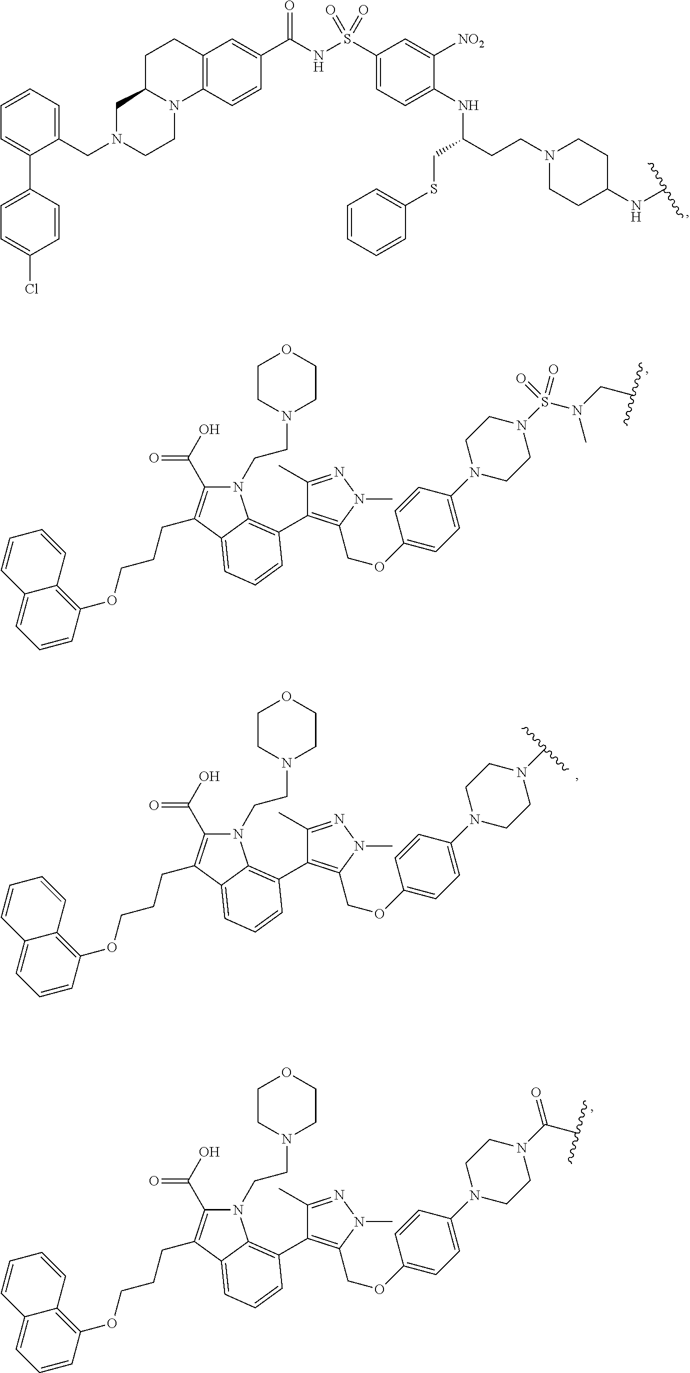 [SMILES: *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1] 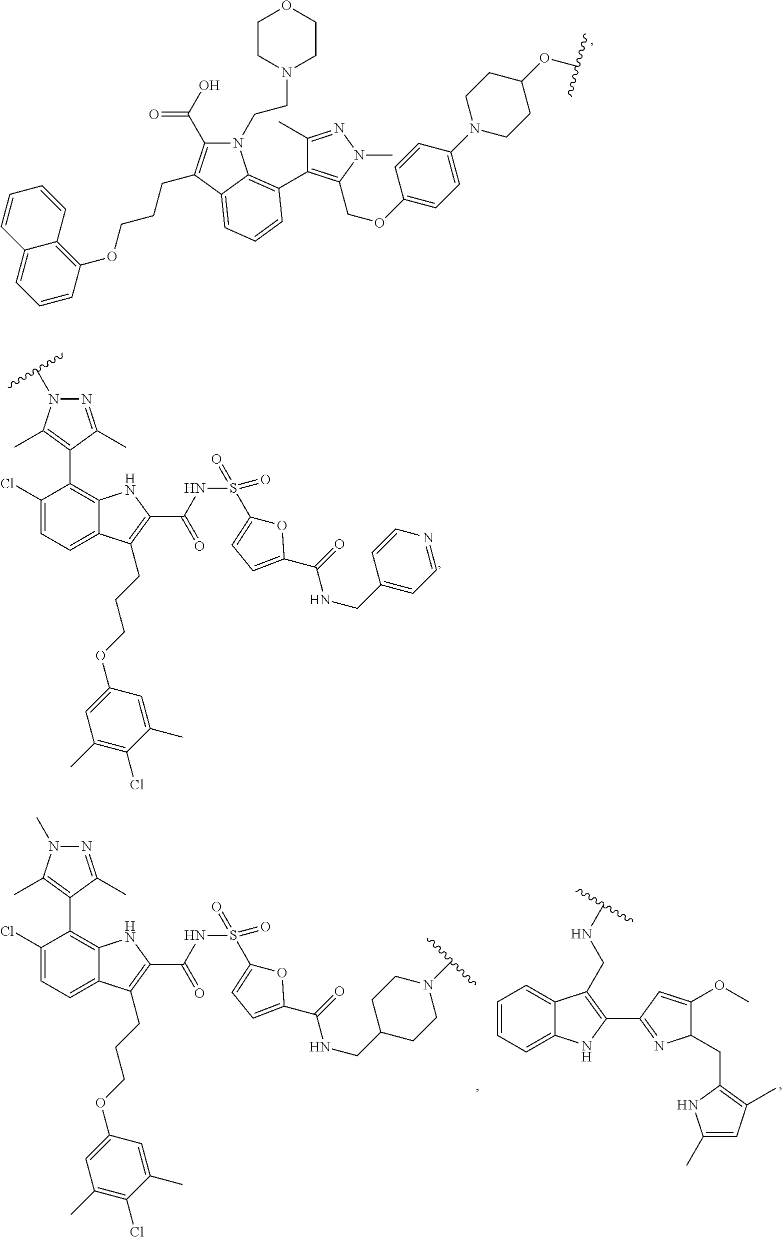 [SMILES: *OC1CCN(c2ccc(OCc3c(-c4cccc5c(CCCOc6cccc7ccccc67)c(C(=O)O)n(CCN6CCOCC6)c45)c(C)nn3C)cc2)CC1 ; *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=NC(Cc3[nH]c(C)cc3C)C(OC)=C2)[nH]c2ccccc12] 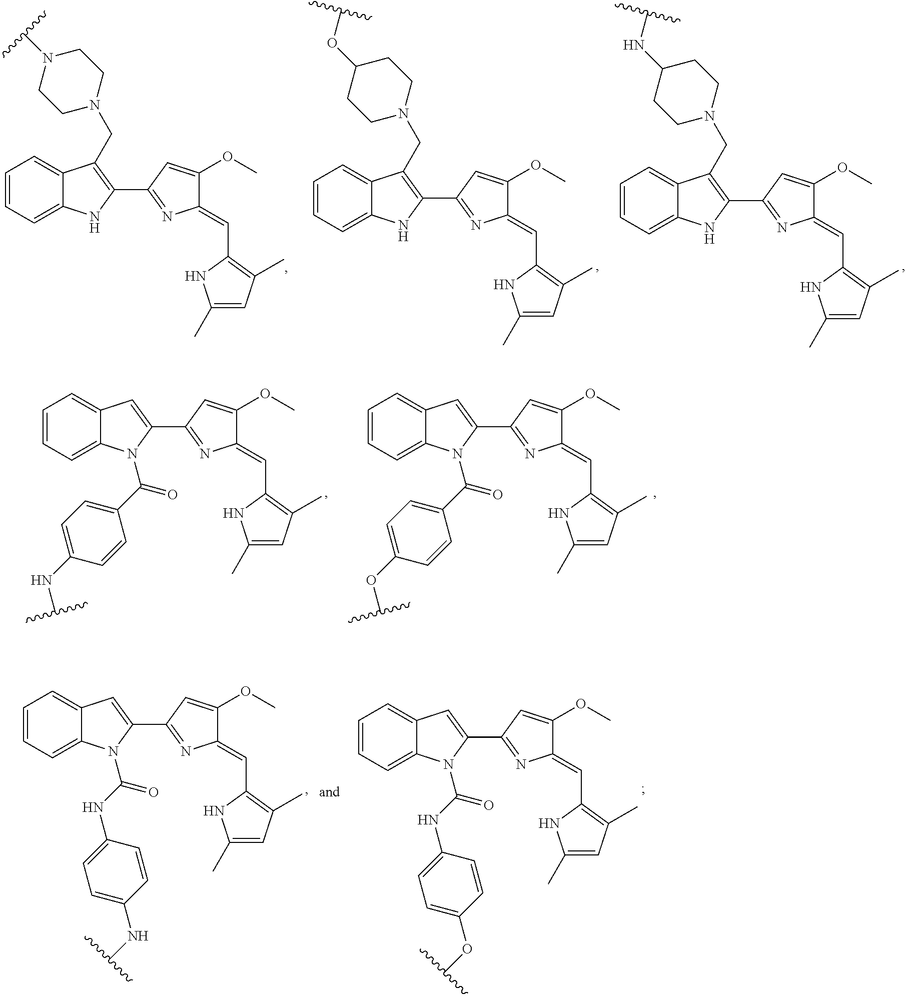
R³ is absent, a bond, or a substituted or unsubstituted C₁-C₁₀ alkyl;
A is absent, a bond, a substituted or unsubstituted C₁-C₆ aryl, a substituted or unsubstituted C₁-C₆cycloalkyl, or a substituted or unsubstituted C₁-C₆ heterocyclic group;
R⁴ is a bond or a substituted or unsubstituted C₁-C₁₀ alky;
n is an integer from 0 to 5,
R² is selected from the group consisting of

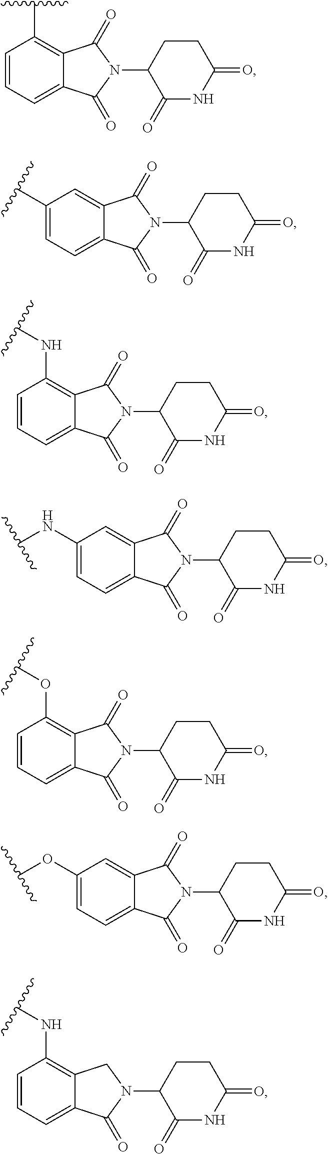

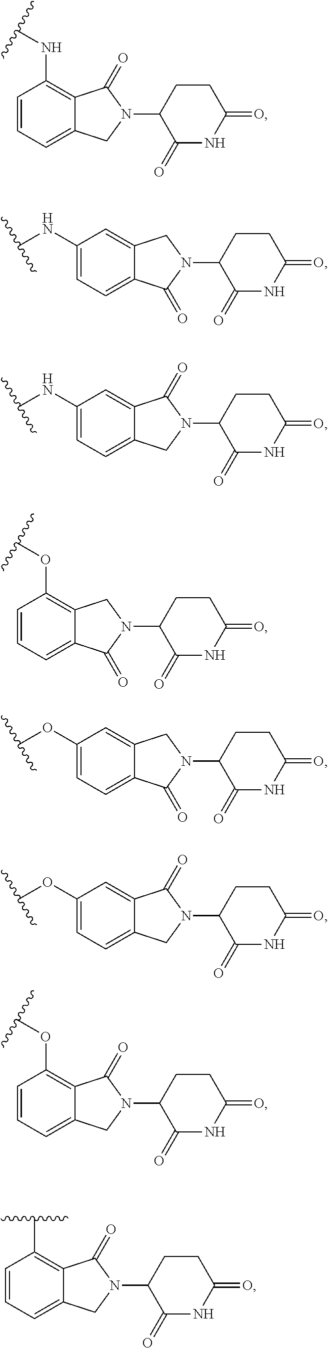

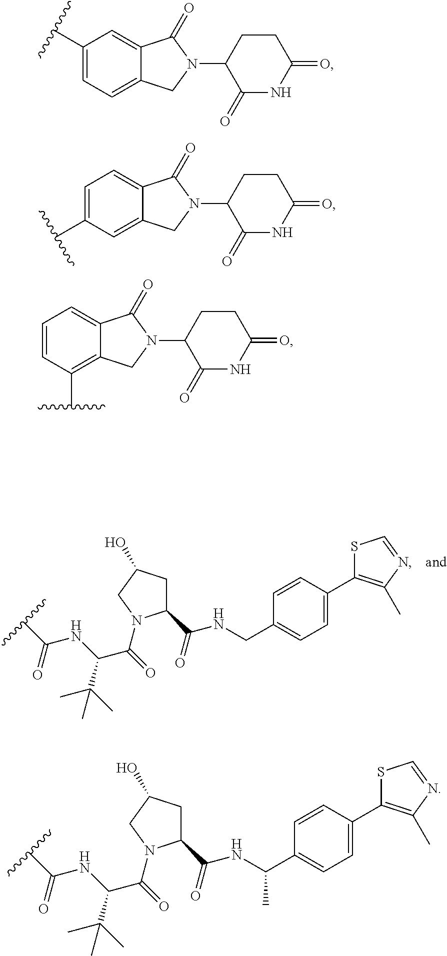

13. The method of claim 12, wherein the bone cells include senescent osteoprogenitors and/or osteocytes.

14. The method of claim 12, wherein there is an increase osteoblast number in the sample or subject.

15. The method of claim 12, wherein the subject is free of thrombocytopenia following administration.

16. A method of treating or preventing osteoporosis in subject in need thereof, the method comprising administering to the sample or subject a composition comprising a therapeutically effective amount of a compound comprising Formula (II): 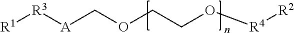
wherein
R¹ is selected from the group consisting of: 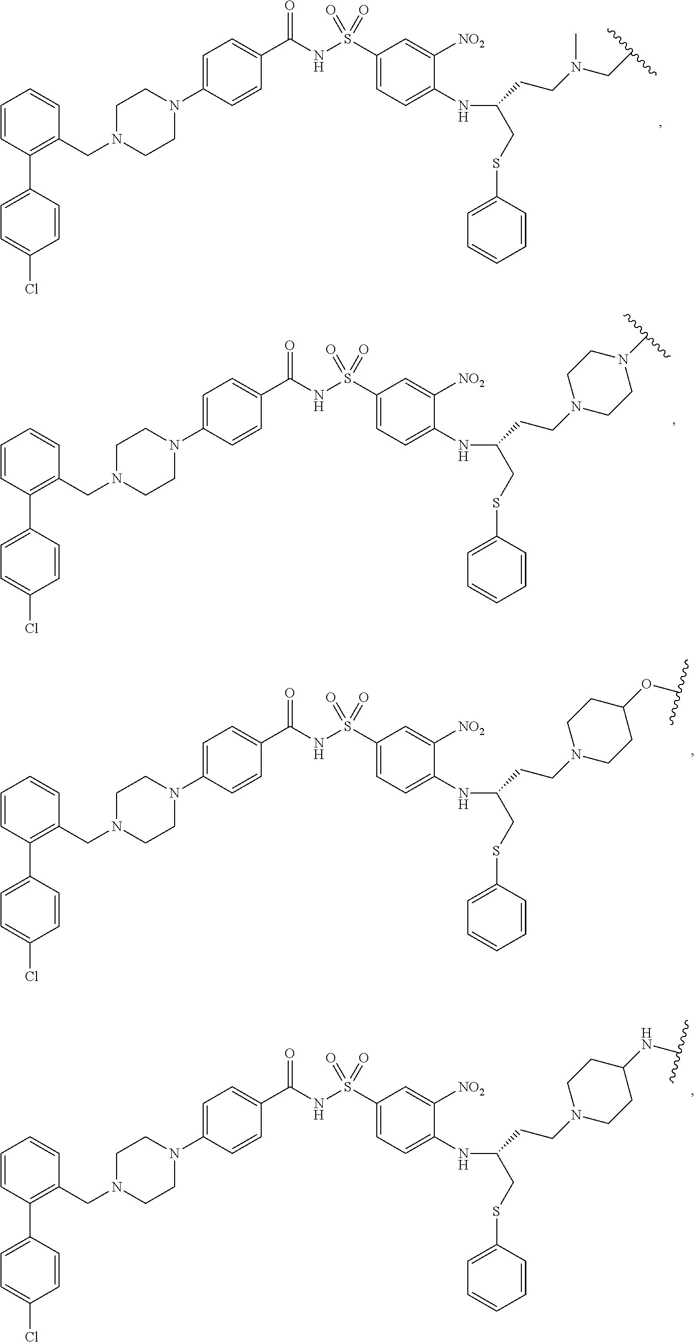 [SMILES: *CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1] 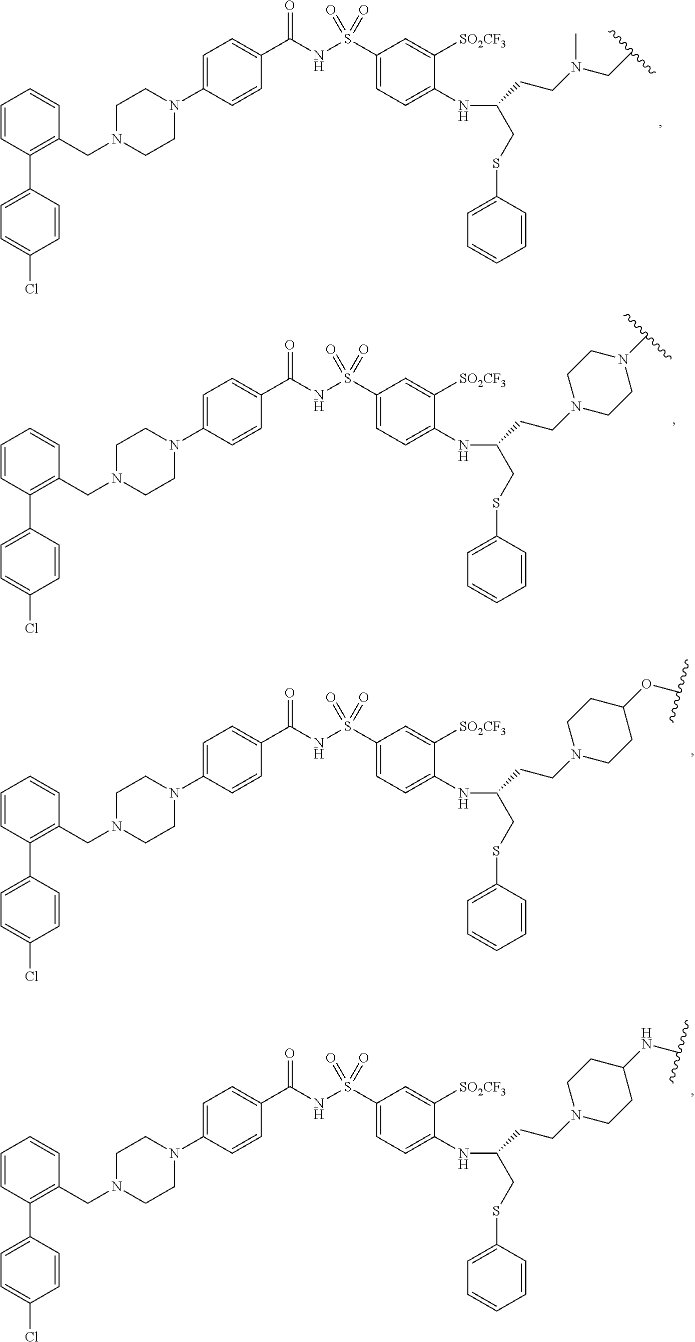 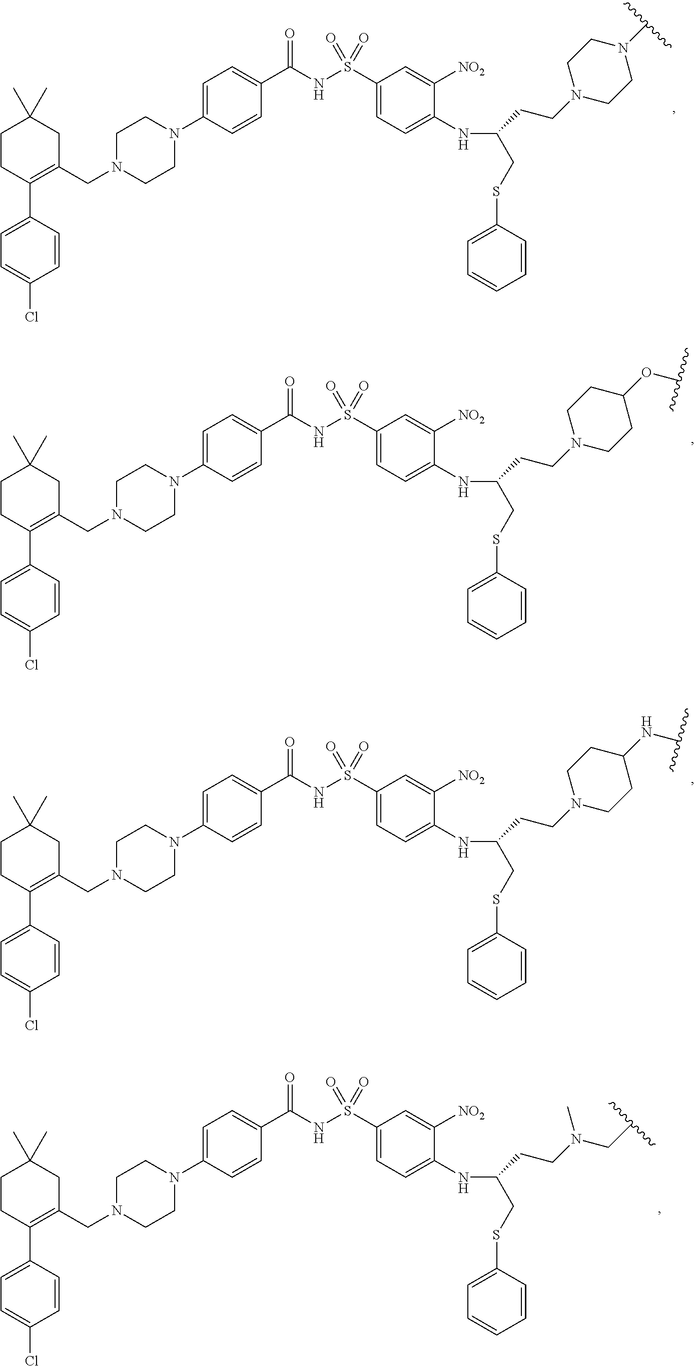 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *OC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *NC1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2[N+](=O)[O-])CC1 ; *CN(C)CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1[N+](=O)[O-]] 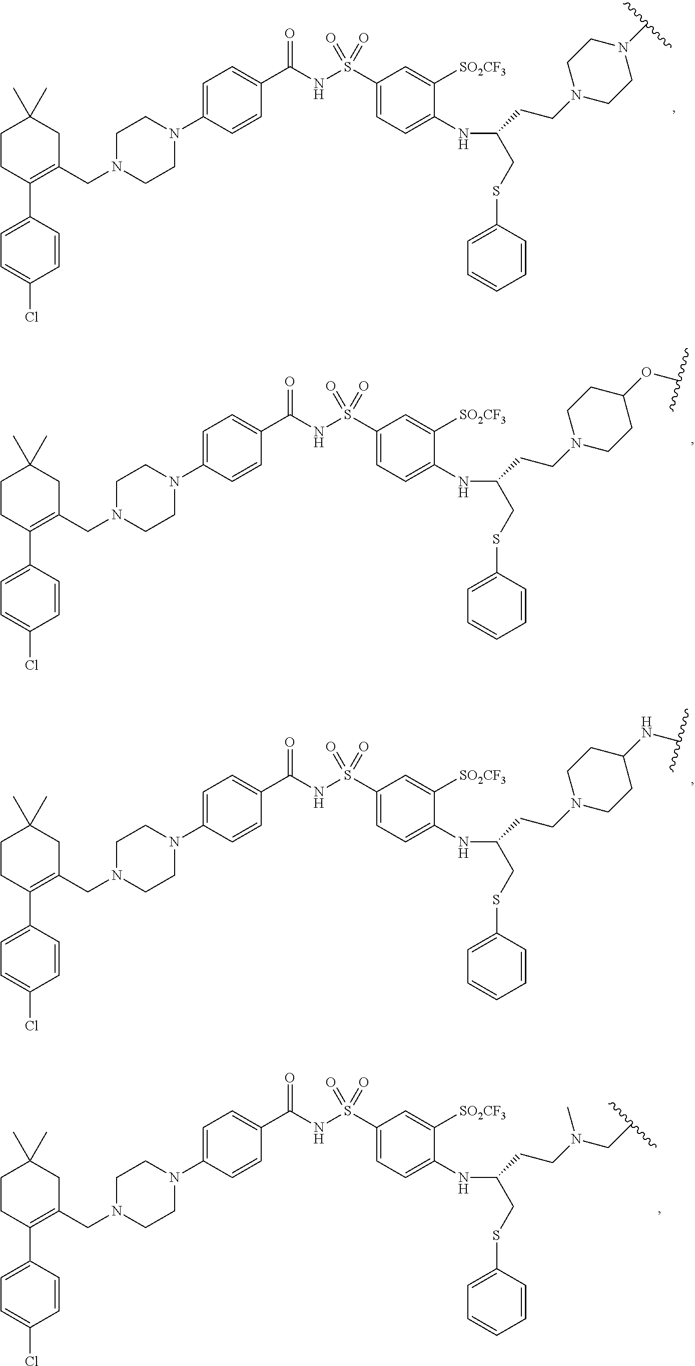 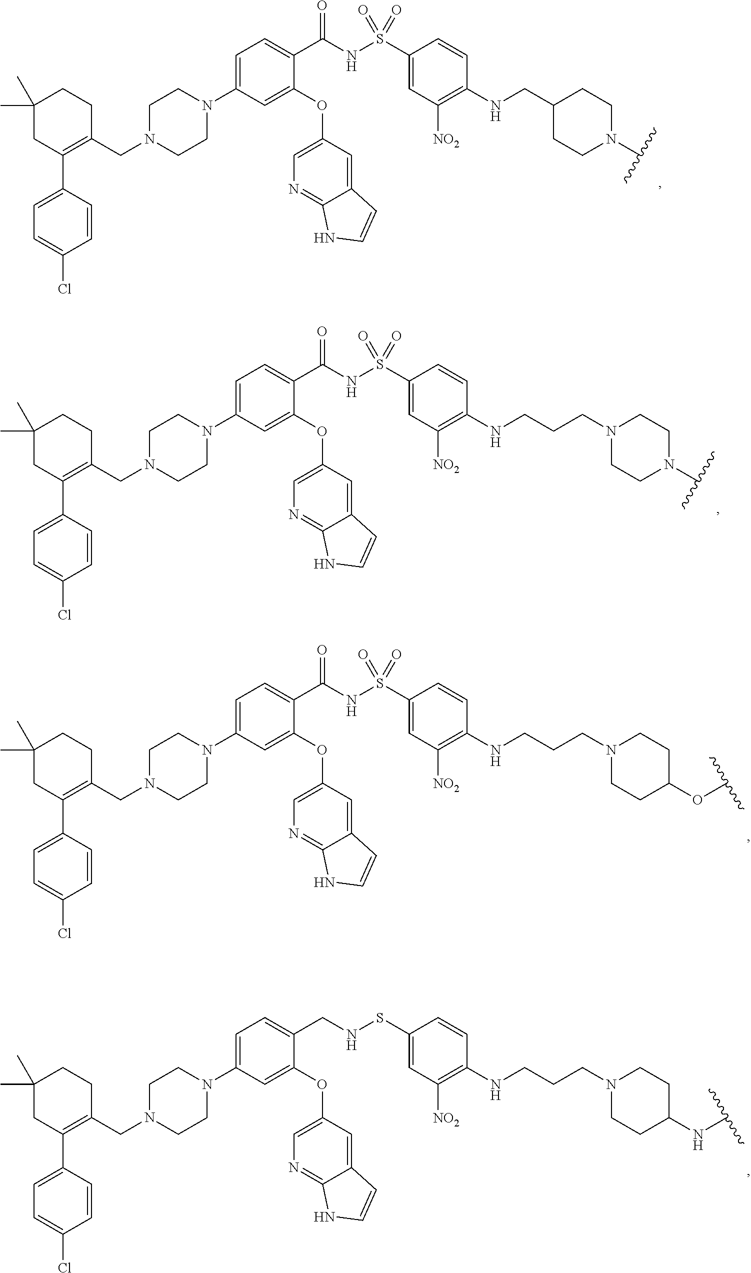 [SMILES: *N1CCC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *OC1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])CC1 ; *NC1CCN(CCCNc2ccc(SNCc3ccc(N4CCN(CC5=C(c6ccc(Cl)cc6)CC(C)(C)CC5)CC4)cc3Oc3cnc4[nH]ccc4c3)cc2[N+](=O)[O-])CC1] 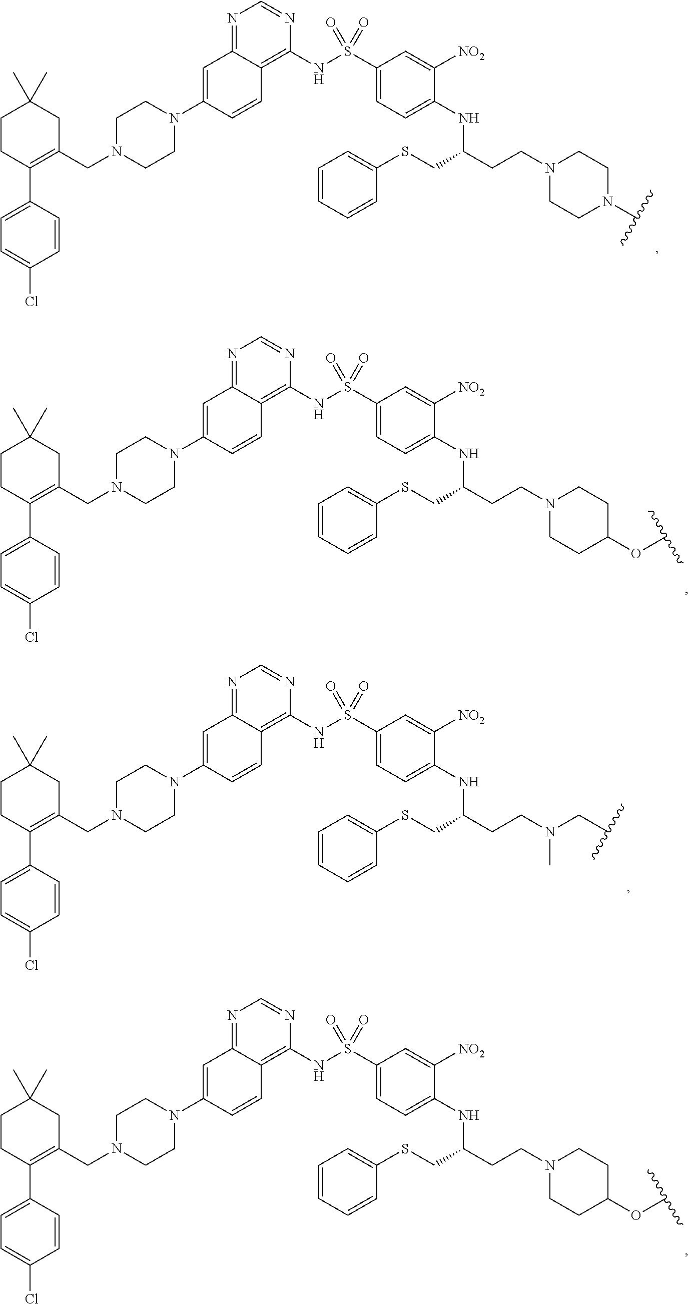 [SMILES: *N1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1 ; *CN(C)CC[C@H](CSc1ccccc1)Nc1ccc(S(=O)(=O)Nc2ncnc3cc(N4CCN(CC5=C(c6ccc(Cl)cc6)CCC(C)(C)C5)CC4)ccc23)cc1[N+](=O)[O-] ; *OC1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2[N+](=O)[O-])CC1] 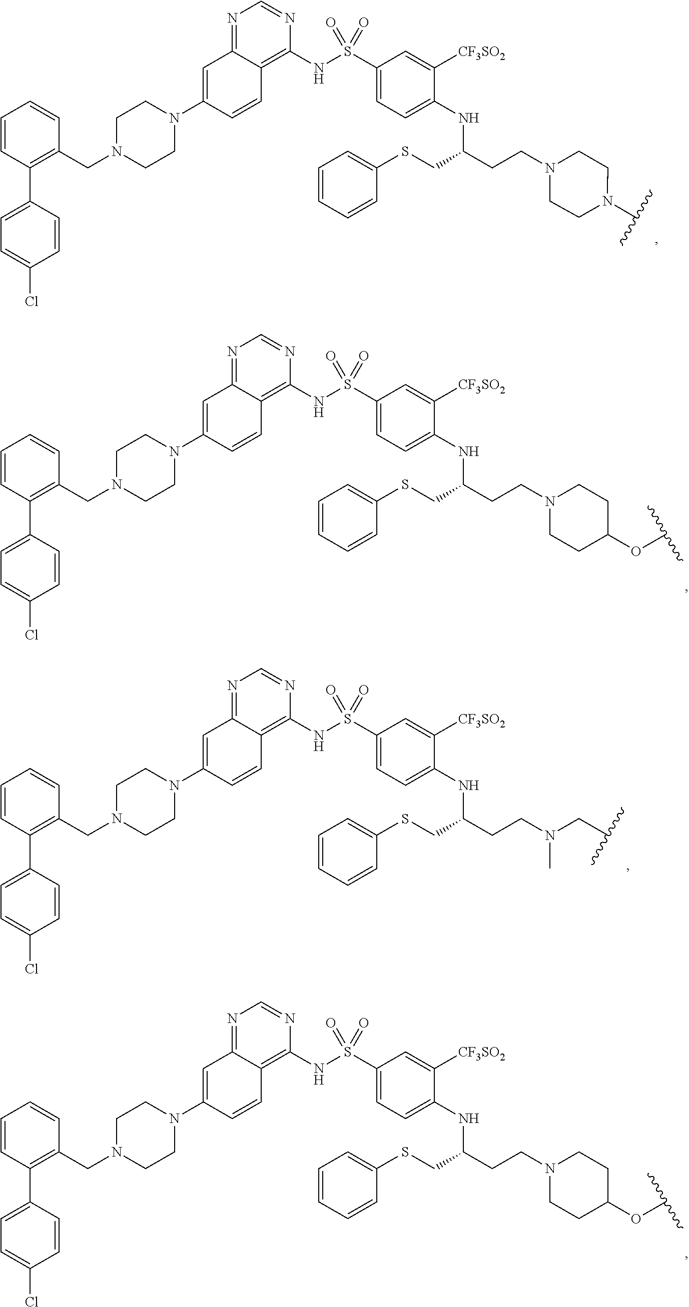 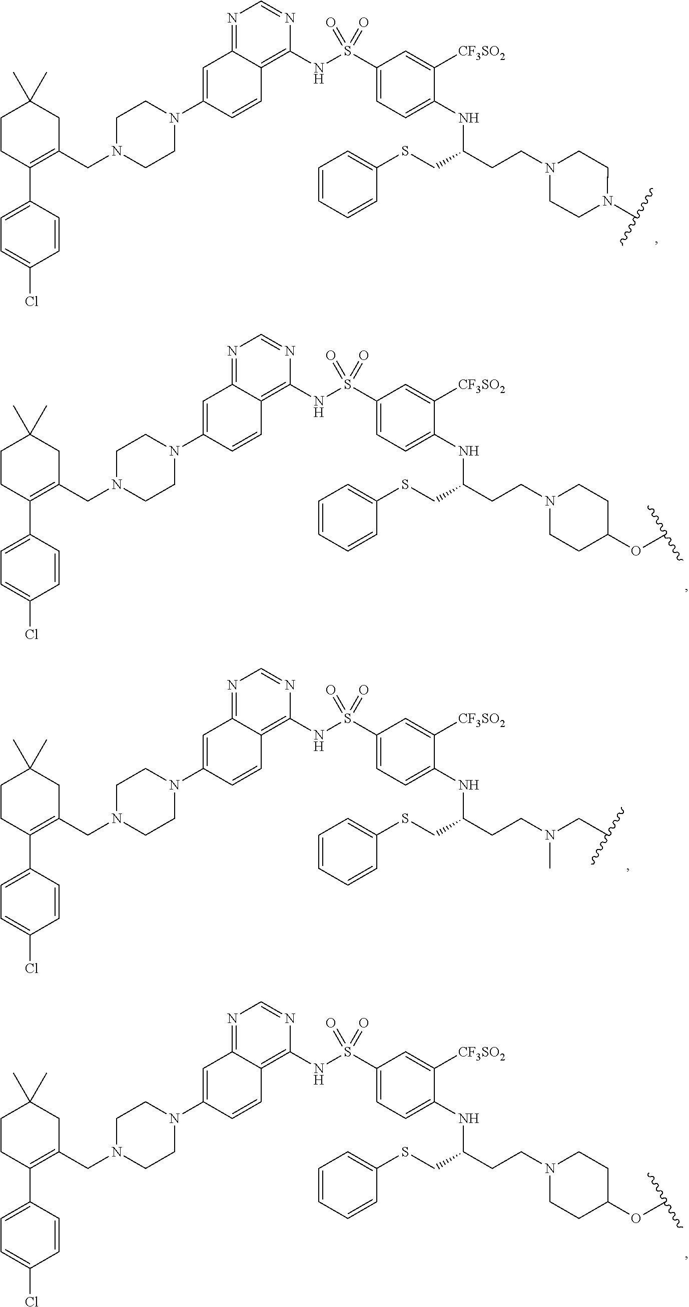 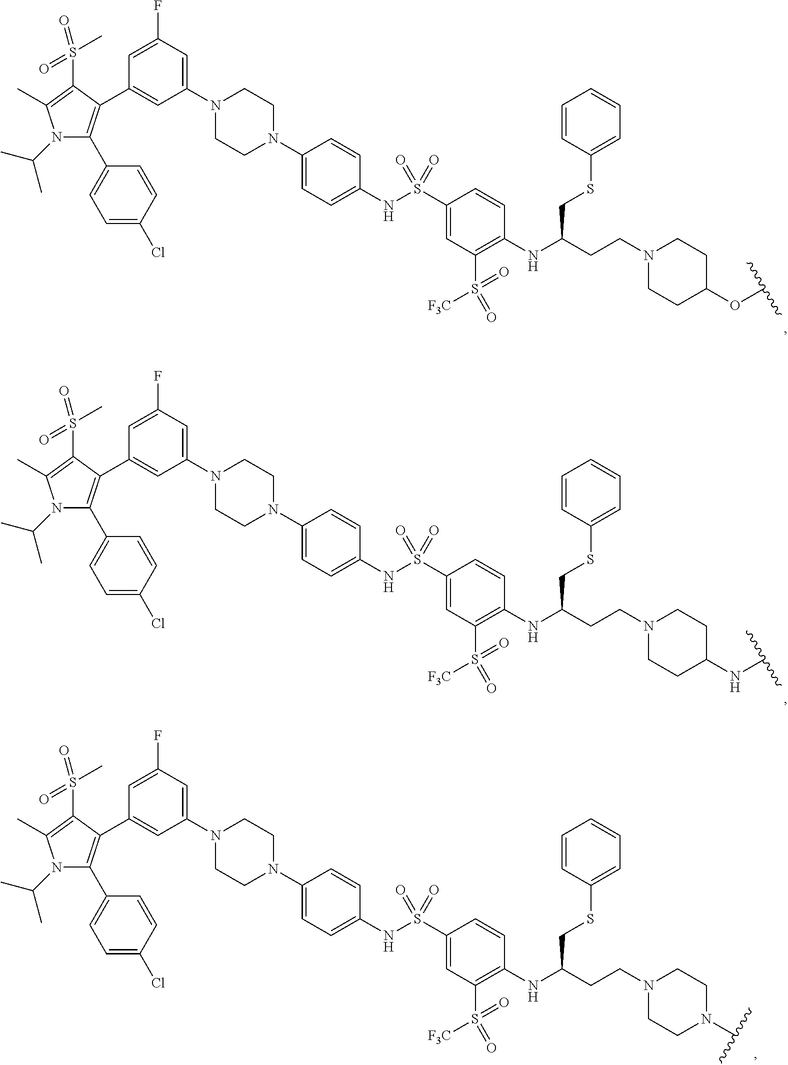 [SMILES: *OC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *NC1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1 ; *N1CCN(CC[C@H](CSc2ccccc2)Nc2ccc(S(=O)(=O)Nc3ccc(N4CCN(c5cc(F)cc(-c6c(S(C)(=O)=O)c(C)n(C(C)C)c6-c6ccc(Cl)cc6)c5)CC4)cc3)cc2S(=O)(=O)C(F)(F)F)CC1] 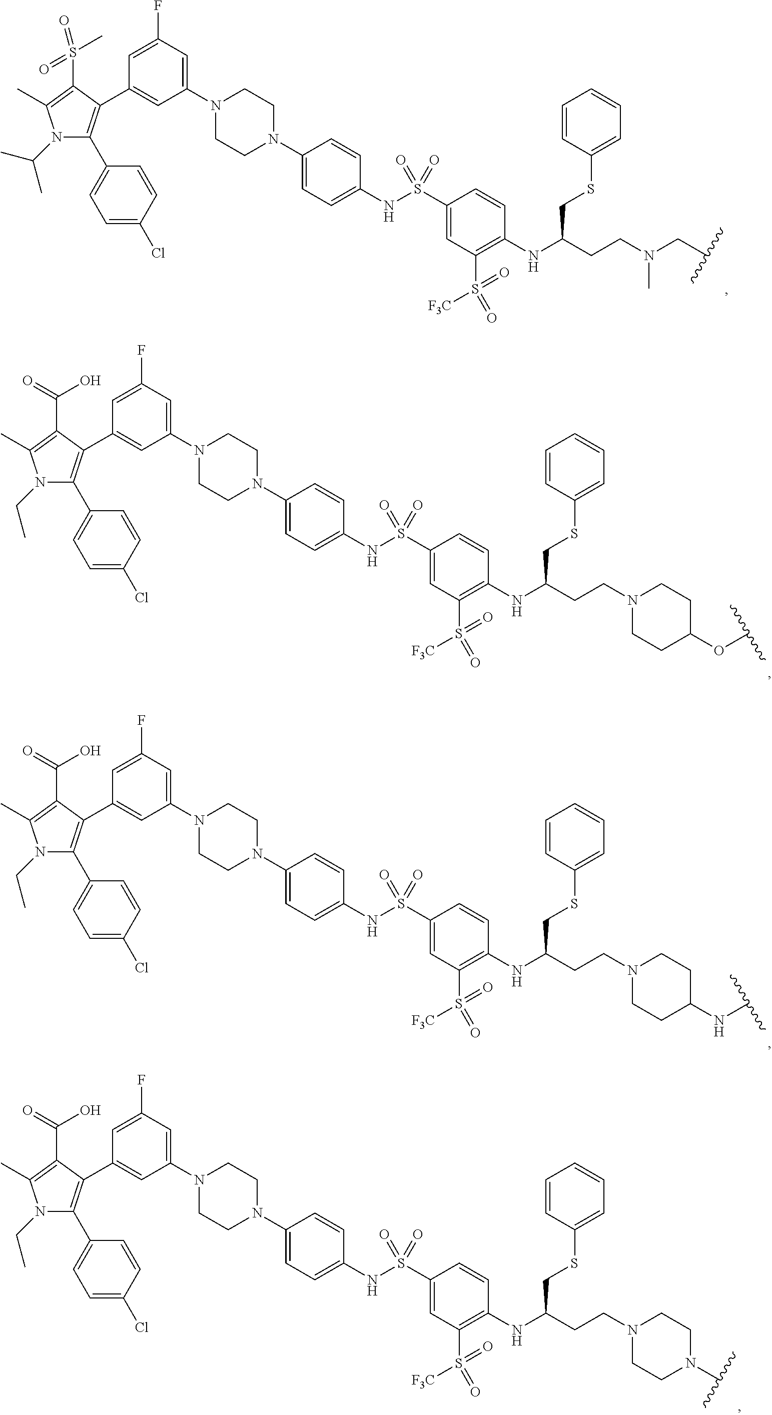 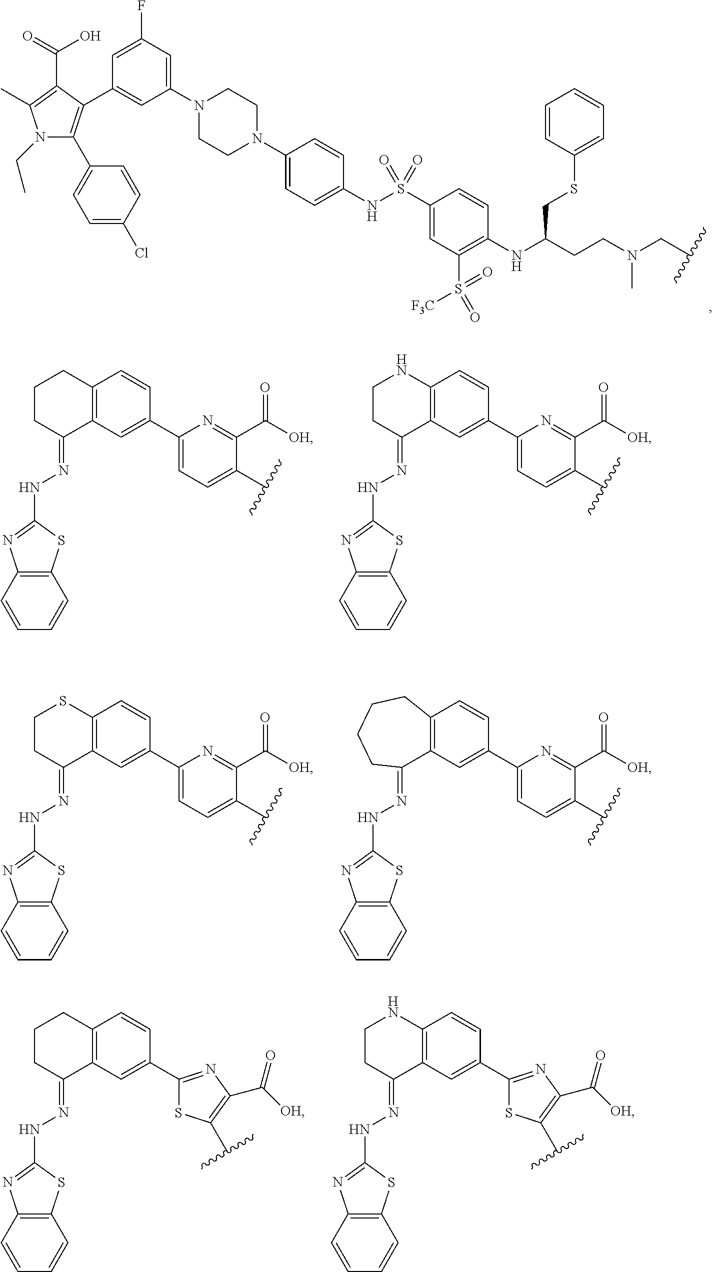 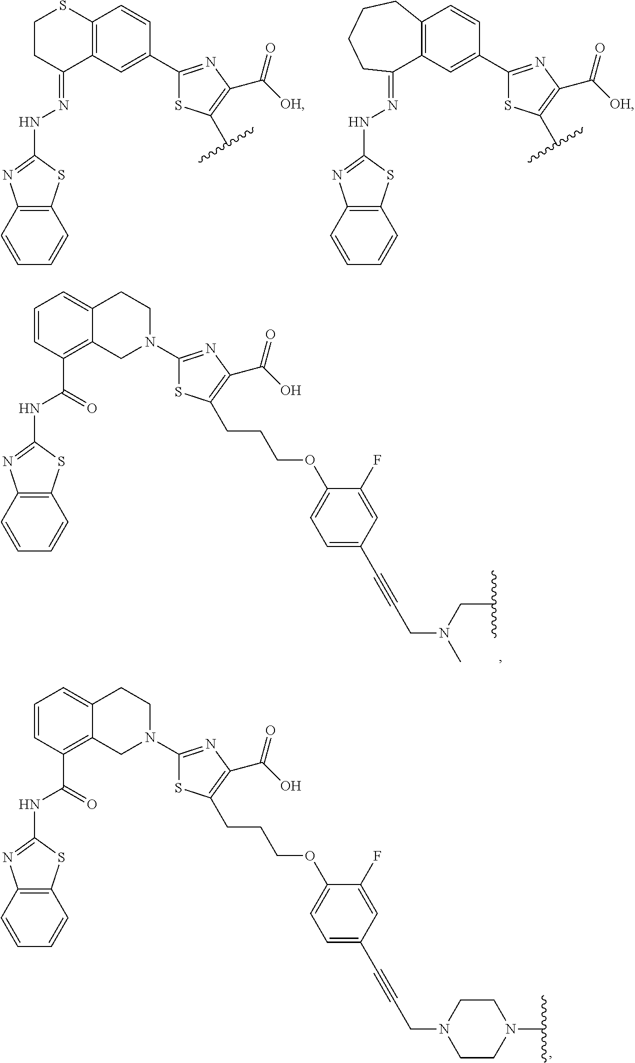 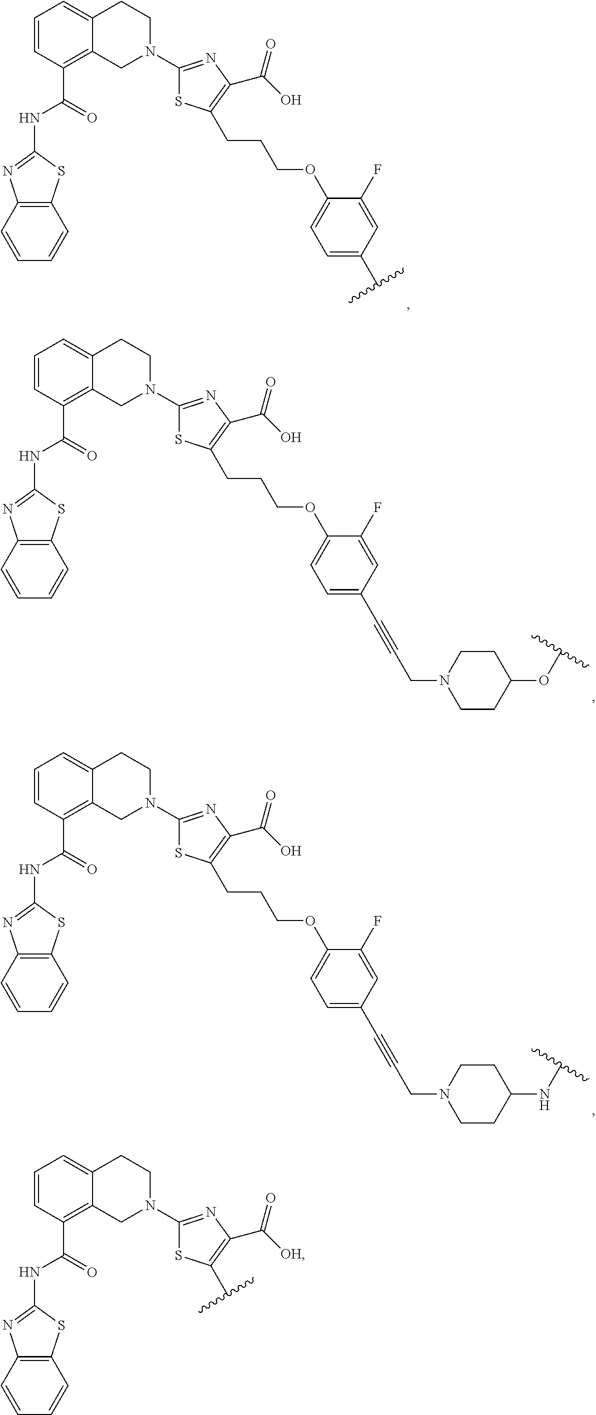 [SMILES: *c1ccc(OCCCc2sc(N3CCc4cccc(C(=O)Nc5nc6ccccc6s5)c4C3)nc2C(=O)O)c(F)c1 ; *OC1CCN(CC#Cc2ccc(OCCCc3sc(N4CCc5cccc(C(=O)Nc6nc7ccccc7s6)c5C4)nc3C(=O)O)c(F)c2)CC1 ; *NC1CCN(CC#Cc2ccc(OCCCc3sc(N4CCc5cccc(C(=O)Nc6nc7ccccc7s6)c5C4)nc3C(=O)O)c(F)c2)CC1 ; *c1sc(N2CCc3cccc(C(=O)Nc4nc5ccccc5s4)c3C2)nc1C(=O)O] 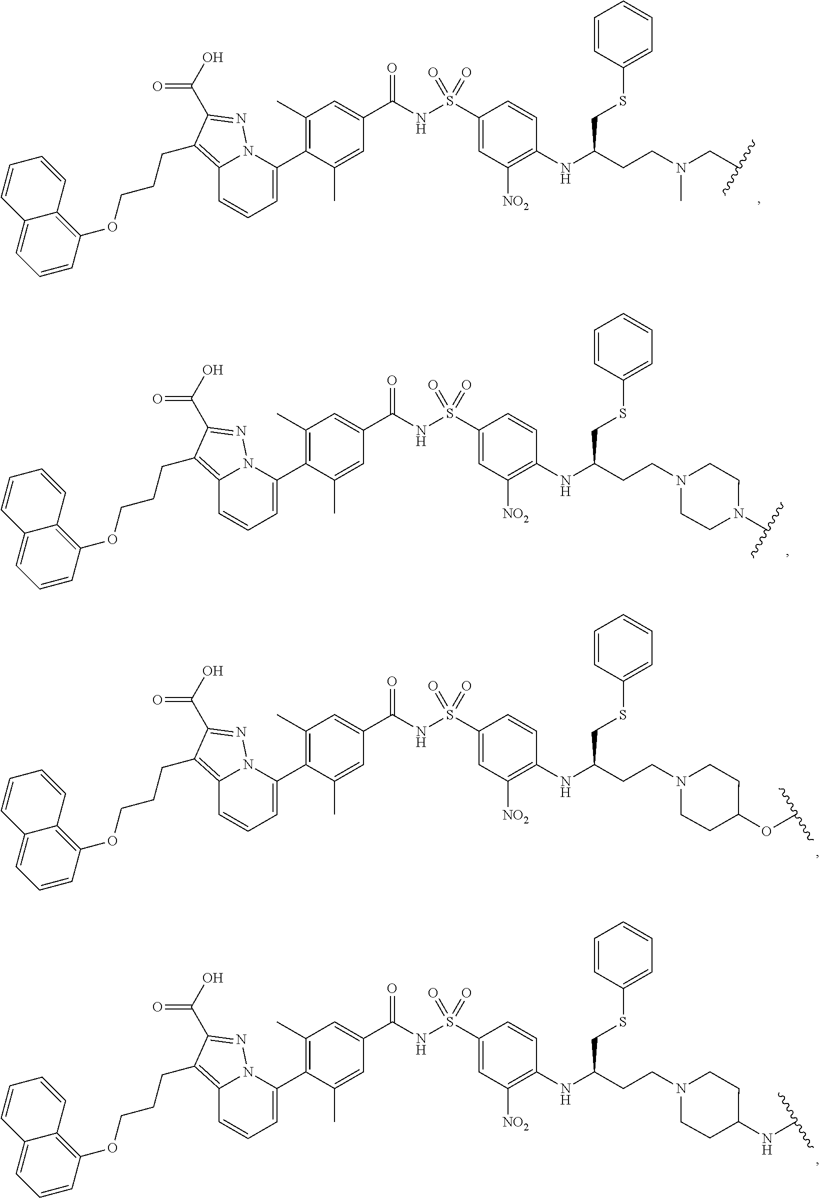 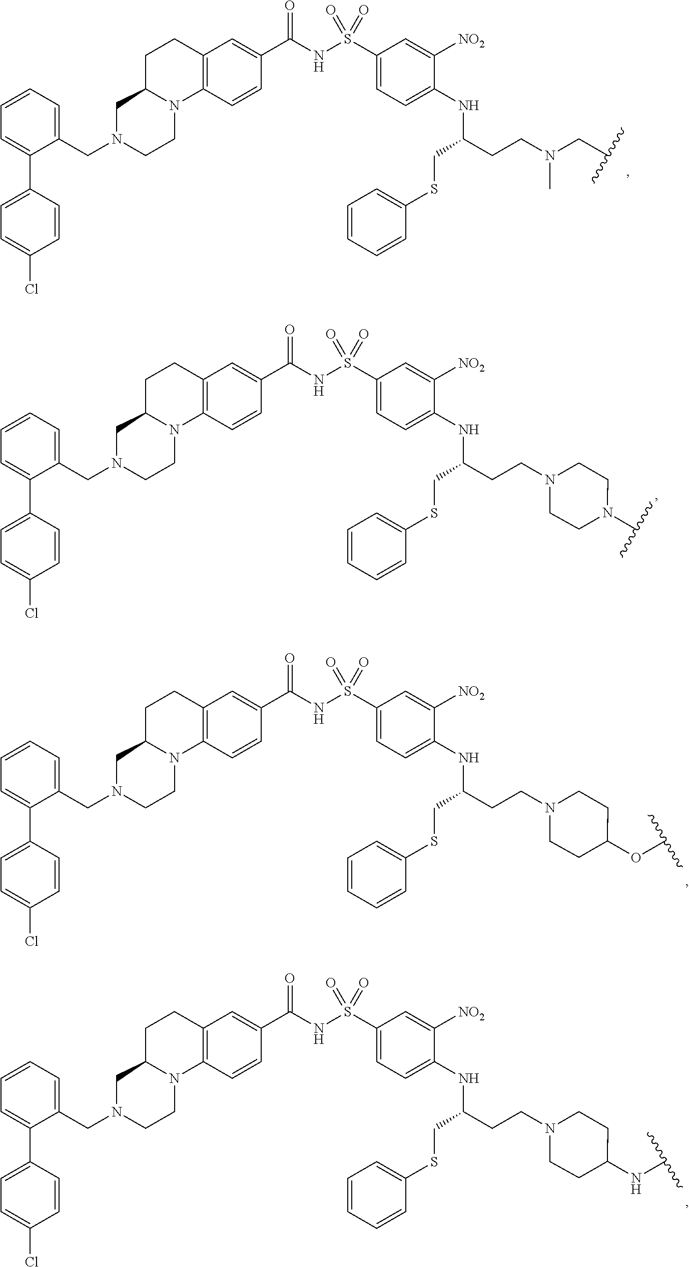 [SMILES: *CN(C)CC[C@H](CSc1ccccc1)Nc1ccc(S(=O)(=O)NC(=O)c2ccc3c(c2)CC[C@@H]2CN(Cc4ccccc4-c4ccc(Cl)cc4)CCN32)cc1[N+](=O)[O-] ; *N1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *OC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1 ; *NC1CCN(CC[C@H](CSc2ccccc2)Nc2ccc(S(=O)(=O)NC(=O)c3ccc4c(c3)CC[C@@H]3CN(Cc5ccccc5-c5ccc(Cl)cc5)CCN43)cc2[N+](=O)[O-])CC1] 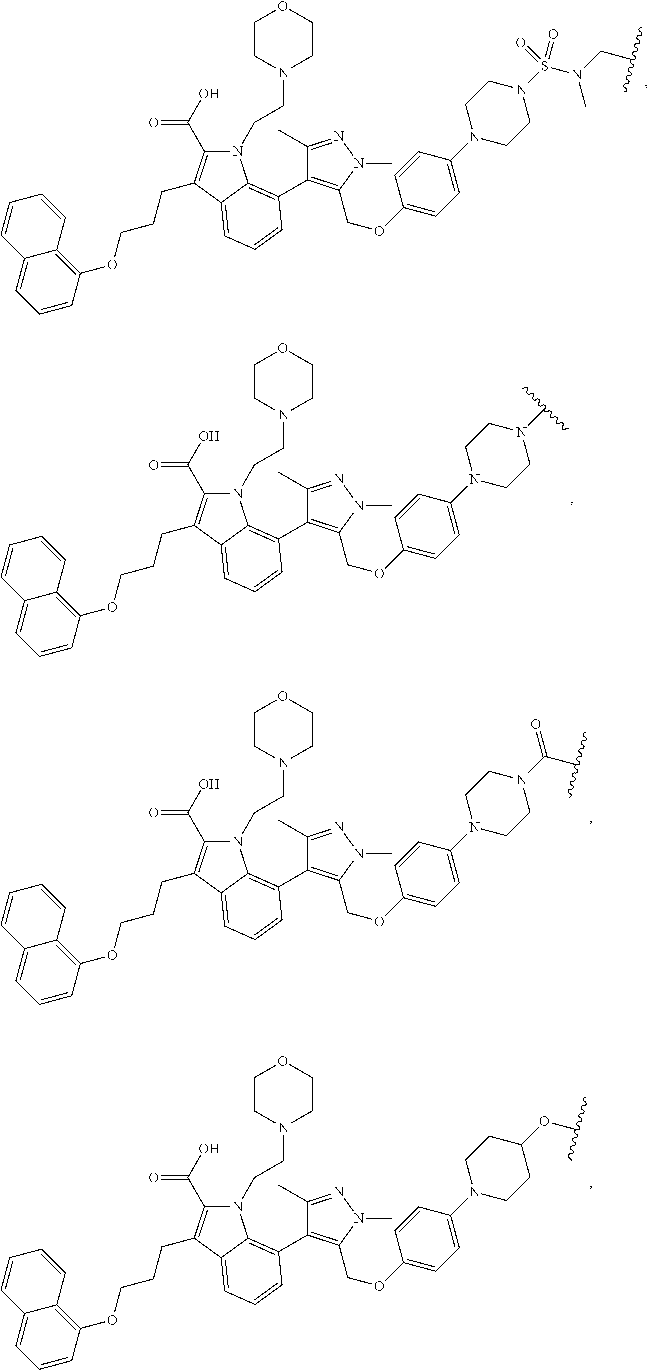 [SMILES: *CN(C)S(=O)(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *C(=O)N1CCN(c2ccc(OCc3c(-c4cccc5c(CCCOc6cccc7ccccc67)c(C(=O)O)n(CCN6CCOCC6)c45)c(C)nn3C)cc2)CC1 ; *OC1CCN(c2ccc(OCc3c(-c4cccc5c(CCCOc6cccc7ccccc67)c(C(=O)O)n(CCN6CCOCC6)c45)c(C)nn3C)cc2)CC1] 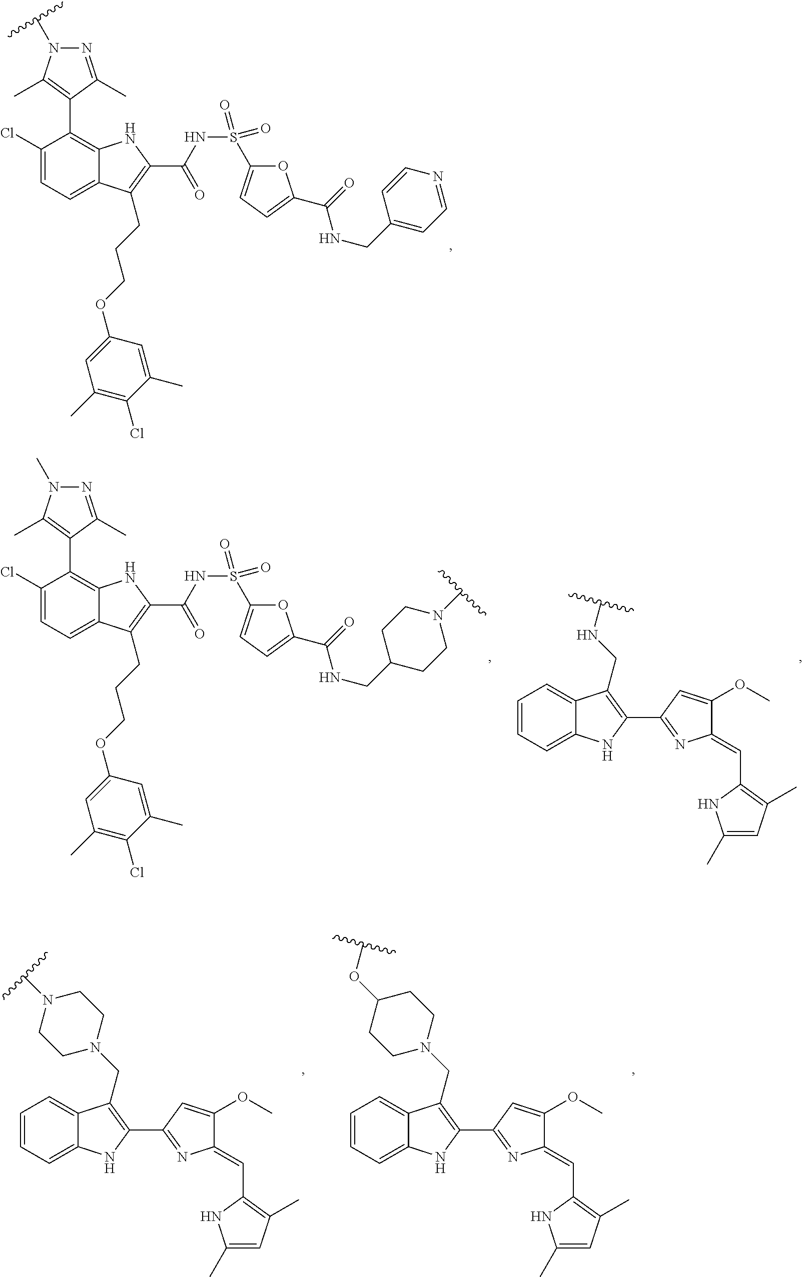 [SMILES: *n1nc(C)c(-c2c(Cl)ccc3c(CCCOc4cc(C)c(Cl)c(C)c4)c(C(=O)NS(=O)(=O)c4ccc(C(=O)NCc5ccncc5)o4)[nH]c23)c1C ; *N1CCC(CNC(=O)c2ccc(S(=O)(=O)NC(=O)c3[nH]c4c(-c5c(C)nn(C)c5C)c(Cl)ccc4c3CCCOc3cc(C)c(Cl)c(C)c3)o2)CC1 ; *NCc1c(C2=N/C(=C\c3[nH]c(C)cc3C)C(OC)=C2)[nH]c2ccccc12 ; *N1CCN(Cc2c(C3=N/C(=C\c4[nH]c(C)cc4C)C(OC)=C3)[nH]c3ccccc23)CC1 ; *OC1CCN(Cc2c(C3=N/C(=C\c4[nH]c(C)cc4C)C(OC)=C3)[nH]c3ccccc23)CC1] 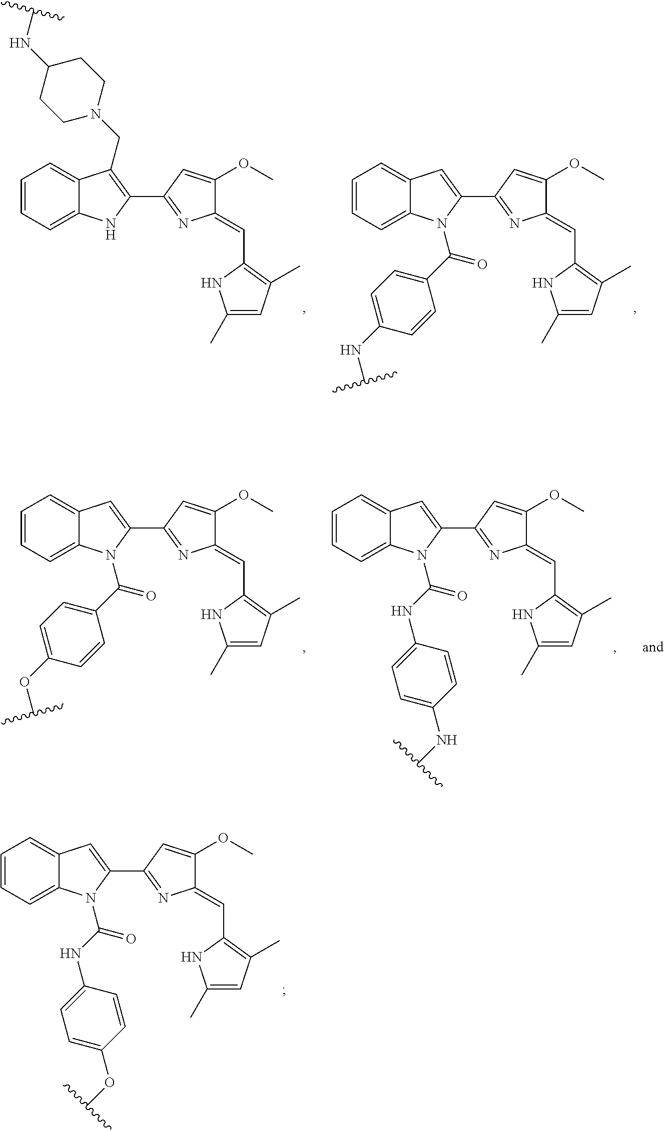 [SMILES: *NC1CCN(Cc2c(C3=N/C(=C\c4[nH]c(C)cc4C)C(OC)=C3)[nH]c3ccccc23)CC1 ; *Nc1ccc(C(=O)n2c(C3=N/C(=C\c4[nH]c(C)cc4C)C(OC)=C3)cc3ccccc32)cc1 ; *Oc1ccc(C(=O)n2c(C3=N/C(=C\c4[nH]c(C)cc4C)C(OC)=C3)cc3ccccc32)cc1 ; *Nc1ccc(NC(=O)n2c(C3=N/C(=C\c4[nH]c(C)cc4C)C(OC)=C3)cc3ccccc32)cc1 ; *Oc1ccc(NC(=O)n2c(C3=N/C(=C\c4[nH]c(C)cc4C)C(OC)=C3)cc3ccccc32)cc1]
R³ is absent, a bond, or a substituted or unsubstituted C₁-C₁₀ alkyl;
A is absent, a bond, a substituted or unsubstituted C₁-C₆ aryl, a substituted or unsubstituted C₁-C₆cycloalkyl, or a substituted or unsubstituted C₁-C₆ heterocyclic group;
R⁴ is a bond or a substituted or unsubstituted C₁-C₁₀ alky;
n is an integer from 0 to 5,
R² is selected from the group consisting of

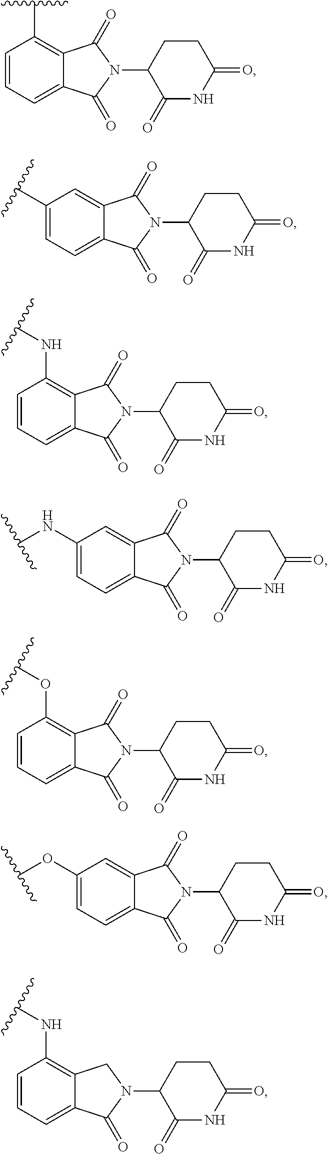

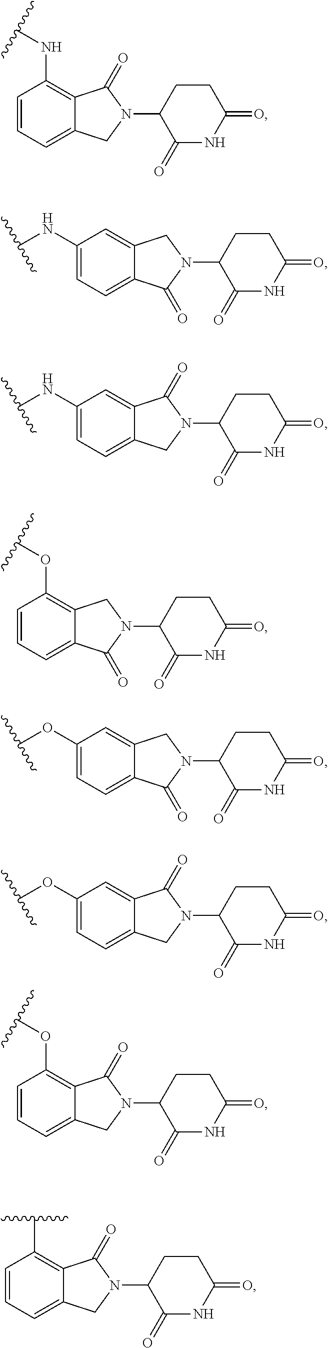

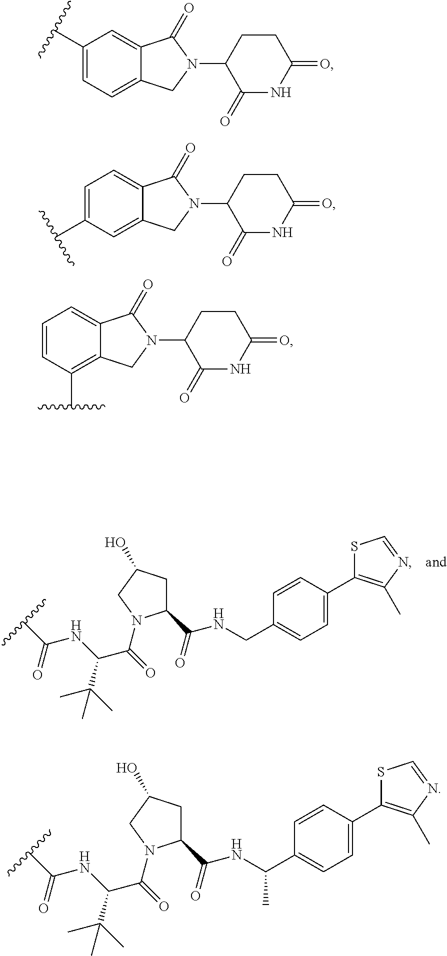

17. The method of claim 16, wherein the subject is free of thrombocytopenia following administration.
